(19) United States
(12) Patent Application Publication
(10) Pub. No.: US 2026/0069605 A1
(43) Pub. Date: Mar. 12, 2026

(54) KRAS INHIBITORS

(71) Applicant: Incyte Corporation, Wilmington, DE (US)

(72) Inventors: Alexander Sokolsky, Wilmington, DE (US); Yannick Boni, Wilmington, DE (US); Gia Hoang, Wilmington, DE (US); Bin Hu, Wilmington, DE (US); Gencheng Li, Wilmington, DE (US); Rocco Policarpo, III, Wilmington, DE (US); Brandon Smith, Wilmington, DE (US); Xiaozhao Wang, Mt. Laurel, NJ (US); Michael Witten, Wilmington, DE (US); Wenhao Zhang, Wilmington, DE (US); Suman Chakrabarty, Wilmington, DE (US)

(21) Appl. No.: 19/325,693

(22) Filed: Sep. 11, 2025

Related U.S. Application Data
(60) Provisional application No. 63/693,384, filed on Sep. 11, 2024.
(60) Provisional application No. 63/724,794, filed on Nov. 25, 2024.
(60) Provisional application No. 63/769,422, filed on Mar. 10, 2025.

(51) Int. Cl. A61K 31/551; A61K 31/506; A61P 35/00; C07D 409/14; C07D 487/04; C07D 487/08; C07D 487/10; C07D 513/04

(52) U.S. Cl. CPC A61K 31/551; A61K 31/506; A61P 35/00; C07D 409/14; C07D 487/04; C07D 487/08; C07D 487/10; C07D 513/04

(57) ABSTRACT

Disclosed are compounds of Formula (I), methods of using the compounds for inhibiting KRAS activity and pharmaceutical compositions comprising such compounds. The compounds are useful in treating, preventing or ameliorating diseases or disorders associated with KRAS activity such as cancer.

### RELATED APPLICATIONS

This application is claims priority to U.S. Provisional Application No. 63/693,384, filed on Sep. 11, 2024, U.S. Provisional Application No. 63/724,794, filed on Nov. 25, 2024, and U.S. Provisional Application No. 63/769,422, filed on Mar. 10, 2025, the contents of which are incorporated in their entirety.

### FIELD OF THE INVENTION

This disclosure provides compounds as well as their compositions and methods of use. The compounds modulate KRAS activity and are useful in the treatment of various diseases including cancer.

### BACKGROUND

Ras proteins are part of the family of small GTPases that are activated by growth factors and various extracellular stimuli. The Ras family regulates intracellular signaling pathways responsible for growth, migration, survival and differentiation of cells. Activation of Ras proteins at the cell membrane results in the binding of key effectors and initiation of a cascade of intracellular signaling pathways within the cell, including the RAF and P13K kinase pathways. Somatic mutations in RAS may result in uncontrolled cell growth and malignant transformation while the activation of RAS proteins is tightly regulated in normal cells (D. Simanshu, et al., Cell, 2017, 170(1), 17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al., Nat. Rev. Drug. Discov., 2014, 13(11), 828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%), and lung adenocarcinomas (23%) (A. D. Cox, et al., Nat. Rev. Drug. Discov., 2014, 13(11), 828-51). In contrast, KRAS G12C mutations predominate in non-small cell lung cancer (NSCLC) comprising 11-16% of lung adenocarcinomas, and 2-5% of pancreatic and colorectal adenocarcinomas (A. D. Cox, et al., Nat. Rev. Drug. Discov., 2014, 13(11), 828-51). Genomic studies across hundreds of cancer cell lines have demonstrated that cancer cells harboring KRAS mutations are highly dependent on KRAS function for cell growth and survival (R. McDonald, et al., Cell, 2017, 170(3), 577-92). The role of mutant KRAS as an oncogenic driver is further supported by extensive in vivo experimental evidence showing mutant KRAS is required for early tumor onset and maintenance in animal models (A. D. Cox, et al., Nat. Rev. Drug. Discov., 2014, 13(11), 828-51).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

### SUMMARY

The present disclosure provides, inter alia, a compound of Formula (I):

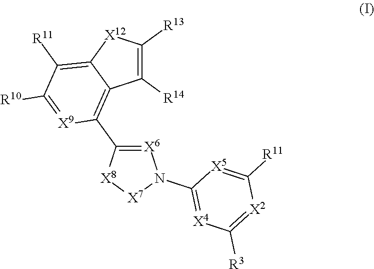

or a pharmaceutically acceptable salt thereof, wherein constituent variables are defined herein.

The present disclosure further provides a pharmaceutical composition comprising a compound of the disclosure, or a pharmaceutically acceptable salt thereof, and at least one pharmaceutically acceptable carrier or excipient.

The present disclosure further provides methods of inhibiting KRAS activity, which comprises administering to an individual a compound of the disclosure, or a pharmaceutically acceptable salt thereof. The present disclosure also provides uses of the compounds described herein in the manufacture of a medicament for use in therapy. The present disclosure also provides the compounds described herein for use in therapy.

The present disclosure further provides methods of treating a disease or disorder in a patient comprising administering to the patient a therapeutically effective amount of a compound of the disclosure, or a pharmaceutically acceptable salt thereof.

The details of one or more embodiments are set forth in the description below. Other features, objects, and advantages will be apparent from the description and from the claims.

### DETAILED DESCRIPTION

For the terms “e.g.” and “such as,” and grammatical equivalents thereof, the phrase “and without limitation” is understood to follow unless explicitly stated otherwise.

The singular forms “a,” “an,” and “the” include plural referents unless the context clearly dictates otherwise.

The term “about” means “approximately” (e.g., plus or minus approximately 10% of the indicated value).

### I. Compounds

In an aspect, provided herein is a compound having Formula (I):

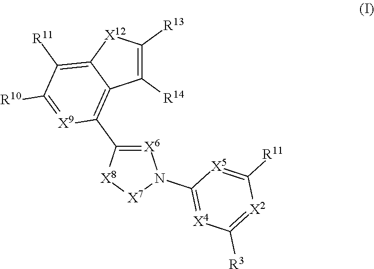

or a pharmaceutically acceptable salt thereof;
   wherein
R¹ is selected from H, D, C₁₋₃ alkyl, C₁₋₃ haloalkyl, C₂₋₃ alkenyl, C₂₋₃ alkynyl, C₃₋₅ cycloalkyl, 4-10 membered heterocycloalkyl, phenyl, 5-10 membered heteroaryl, C₃₋₆ cycloalkyl-C₁₋₃ alkylene, 4-10 membered heterocycloalkyl-C₁₋₃ alkylene, phenyl-C₁₋₃ alkylene, 5-10 membered heteroaryl-C₁₋₃alkylene, halo, CN, OR^{a1}, C(O)R^{b1}, C(O)NR^{c1}R^{d1}, NR^{c1}R^{e1}, and NR^{c1}C(O)R^{b1}; wherein the C₃₋₅ cycloalkyl, 4-10 membered heterocycloalkyl, phenyl, 5-10 membered heteroaryl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene forming R¹ are each optionally substituted with 1, 2, or 3 substituents independently selected from R^{1A}; and wherein the C₁₋₃ alkyl, C₂₋₃ alkenyl, and C₂₋₃ alkynyl forming R¹ are each optionally substituted with 1, 2, or 3 substituents independently selected from R^{1B};
each R^{1A} is independently selected from C₁₋₃ alkyl, C₁₋₃ haloalkyl, C₂₋₃ alkenyl, C₂₋₃ alkynyl, and R^{1B}; wherein the C₁₋₃ alkyl, C₂₋₃ alkenyl, and C₂₋₃ alkynyl forming R^{1A} are each optionally substituted with 1, 2, or 3 substituents each independently selected from R^{1B};
each R^{1B} is independently selected from D, C₃₋₆cycloalkyl, 4-10 membered heterocycloalkyl, phenyl, 5-6 membered heteroaryl, halo, CN, OR^{a1B}, C(O)R^{b1B}, NR^{c1B}C(O)R^{b1B}, C(O)NR^{c1B}R^{d1B}C(O)OR^{a1B}, NR^{c1B}R^{d1B}, and S(O)₂R^{b1B}; wherein the C₃₋₆ cycloalkyl, 4-6 membered heterocycloalkyl, phenyl, and 5-6 membered heteroaryl forming R^{1B} are each optionally substituted with 1, 2, or 3 substituents independently selected from R^{1C};
each R^{1C} is independently selected from D, C₃₋₆ cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C}; wherein the C₃₋₆cycloalkyl, 4-6 membered heterocycloalkyl, phenyl, and 5-6 membered heteroaryl forming R^{1C} are each optionally substituted with 1, 2, or 3 substituents independently selected from R^{1D};
each R^{1D} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a1D}, C(O)R^{b1D}, C(O)NR^{c1D}R^{d1D}, C(O)OR^{a1D}, NR^{c1D}R^{d1D}, and S(O)₂R^{b1D};
each R^{a1} is independently selected from C₁₋₆ alkyl, C₁₋₃ haloalkyl, C₂₋₆ alkenyl, C₂₋₆ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a1} are each optionally substituted with 1, 2, or 3 substituents independently selected from R^{1A}; wherein the C₁₋₆ alkyl, C₂₋₆ alkenyl, and C₂₋₆ alkynyl forming R^{a1} are each optionally substituted with 1, 2, or 3 substituents independently selected from R^{1B};
each R^{b1}, R^{c1}, and R^{d1} is independently selected from H, C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; and wherein the C₃₋₆ cycloalkyl, 4-6 membered heterocycloalkyl, phenyl, and 5-6 membered heteroaryl forming R^{b1}, R¹, and R^{d1} are each optionally substituted with 1, 2, or 3 substituents independently selected from R^{1A}; wherein the C₁₋₃ alkyl, C₁₋₃ haloalkyl, C₂₋₃ alkenyl, and C₂₋₃ alkynyl, forming R^{b1}, R^{c1}, and R^{d1} are each optionally substituted with 1, 2, or 3 substituents independently selected from R^{1B}; or any R^{c1} and R^{d1} attached to the same N atom, together with the N atom to which they are attached, form a 4-, 5-, or 6-membered heterocycloalkyl group optionally substituted with 1, 2, or 3 substituents independently selected from R^{1A};
each R^{e1} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{e1} are each optionally substituted with 1, 2, or 3 substituents independently selected from R^{1A}; wherein the C₁₋₃ alkyl, C₁₋₃ haloalkyl, C₂₋₃ alkenyl, and C₂₋₃ alkynyl forming R^{e1} are each optionally substituted with 1, 2, or 3 substituents independently selected from R^{1B}; or
R^{c1} and R^{e1} attached to the same N atom, together with the N atom to which they are attached, form a 4-, 5-, or 6-membered heterocycloalkyl group optionally substituted with 1, 2, or 3 substituents independently selected from R^{1A};
each R^{a1B}, R^{b1B}, R^{c1B} and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1C}, R^{b1C}, R^{c1C}, and R^{d1C} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1D}, R^{b1D}, R^{c1D}, and R^{d1D} is independently selected from H, C₁₋₃ alkyl, and C₁₋₃ haloalkyl;
X² is N or CR²;
R² is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, halo, CN, OR^{2A}, and NR^{2B}R^{2C}; wherein the C₃₋₁₀ cycloalkyl and 4-10 membered heterocycloalkyl forming R² are each optionally substituted with 1, 2, or 3 substituents independently selected from R^{2D}; and wherein the C₁₋₃ alkyl, C₂₋₃ alkenyl, and C₂₋₃ alkynyl forming R² are each optionally substituted with 1, 2, or 3 substituents independently selected from R^{2E};
R^{2A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{2B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{2C} is selected from H, C₁₋₃ alkyl, C₂₋₃ alkenyl, and C₂₋₃ alkynyl; or
R^{2B} and R^{2C}, together with the N atom to which they are both attached, optionally form a 4-, 5-, 6-, or 7-membered heterocycloalkyl group that is optionally substituted with 1, 2, or 3 substituents independently selected from independently selected from R^{2D};
each R^{2D} is independently selected from C₁₋₃ alkyl, C₁₋₃ haloalkyl, C₂₋₃ alkenyl, and C₂₋₃ alkynyl;
each R^{2E} is independently selected from D, halo, CN, OH, NH₂, NH(C₁₋₃ alkyl), and N(C₁₋₃ alkyl)₂;
R³ is selected from H, D, C₁₋₃ alkyl, C₁₋₃ haloalkyl, C₂₋₃ alkenyl, C₂₋₃ alkynyl, C₃₋₅ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a3}, C(O)R^{b3}, C(O)NR^{c3}R^{d3}, NR^{c3}R^{e3}, and NR^{c3}C(O)R^{b3}; wherein the C₃₋₅ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R³ are each optionally substituted with 1, 2, or 3 substituents independently selected from R^{3A}; wherein the C₁₋₃ alkyl, C₂₋₃ alkenyl, and C₂₋₃ alkynyl forming R³ are each optionally substituted with 1, 2, or 3 substituents independently selected from R^{3B};
each R^{3A} is independently selected from C₁₋₃ alkyl, C₁₋₃ haloalkyl, C₂₋₃ alkenyl, C₂₋₃ alkynyl, and R^{3B}; wherein the C₁₋₃ alkyl, C₂₋₃ alkenyl, and C₂₋₃ alkynyl forming R^{3A} are each optionally substituted with 1, 2, or 3 substituents each independently selected from R^{3B};
each R^{3B} is independently selected from D, C₃₋₆ cycloalkyl, 4-10 membered heterocycloalkyl, phenyl, 5-6 membered heteroaryl, halo, CN, C(O)R^{b3B}, C(O)NR^{c3B}R^{d3B} NR^{c3B}C(O)R^{b3B}, C(O)OR^{a3B}, NR^{c3B}R^{d3B}, and S(O)₂R^{b3B}; wherein the C₃₋₆ cycloalkyl, 4-6 membered heterocycloalkyl, phenyl, and 5-6 membered heteroaryl forming R^{3B} are each optionally substituted with 1, 2, or 3 substituents independently selected from R^{3C};
each R^{3C} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a3C}, C(O)R^{b3C}, C(O)NR^{c3C}R^{d3C}, C(O)OR^{a3C}, NR^{c3C}R^{d3C}, and S(O)₂R^{b3C};
each R^{a3} is independently selected from C₁₋₆ alkyl, C₁₋₃ haloalkyl, C₂₋₆ alkenyl, C₂₋₆ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a3} are each optionally substituted with 1, 2, or 3 substituents independently selected from R^{3A}; wherein the C₁₋₆ alkyl, C₂₋₆ alkenyl, and C₂₋₆ alkynyl forming R^{a3} are each optionally substituted with 1, 2, or 3 substituents independently selected from R^{3B};
each R^{b3}, R^{c3}, and R^{d3} is independently selected from H, C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; and wherein the C₃₋₆ cycloalkyl, 4-6 membered heterocycloalkyl, phenyl, and 5-6 membered heteroaryl forming R^{b3}, R^{c3}, and R^{d3} are each optionally substituted with 1, 2, or 3 substituents independently selected from R^{3A}; wherein the C₁₋₃ alkyl, C₁₋₃ haloalkyl, C₂₋₃ alkenyl, and C₂₋₃ alkynyl, forming R^{b3}, R^{c3}, and R^{d3} are each optionally substituted with 1, 2, or 3 substituents independently selected from R^{3B}; or
any R^{c3} and R^{d3} attached to the same N atom, together with the N atom to which they are attached, form a 4-, 5-, or 6-membered heterocycloalkyl group optionally substituted with 1, 2, or 3 substituents independently selected from R^{3A};
each R^{e3} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{e3} are each optionally substituted with 1, 2, or 3 substituents independently selected from R^{3A}; wherein the C₁₋₃ alkyl, C₁₋₃ haloalkyl, C₂₋₃ alkenyl, and C₂₋₃ alkynyl forming R^{e3} are each optionally substituted with 1, 2, or 3 substituents independently selected from R^{3B}; or
R^{c3} and R^{e3} attached to the same N atom, together with the N atom to which they are attached, form a 4-, 5-, or 6-membered heterocycloalkyl group optionally substituted with 1, 2, or 3 substituents independently selected from R^{3A};
each R^{a3B}, R^{b3B}, R^{c3B} and R^{d3B} is independently selected from H, C₁₋₃ alkyl, and C₁₋₃ haloalkyl;
each R^{a3C}, R^{b3C}, R^{c3C}, and R^{d3C} is independently selected from H, C₁₋₃ alkyl, and C₁₋₃ haloalkyl;
X⁴ is N or CR⁴;
R⁴ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, halo, CN, OR^{4A}, and NR^{4B}R^{4C}; wherein the C₃₋₁₀ cycloalkyl and 4-10 membered heterocycloalkyl forming R⁴ are each optionally substituted with 1, 2, or 3 substituents independently selected from R^{4D}; and wherein the C₁₋₃ alkyl, C₂₋₃ alkenyl, and C₂₋₃ alkynyl forming R⁴ are each optionally substituted with 1, 2, or 3 substituents independently selected from R^{4E};
R^{4A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{4B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{4C} is selected from H, C₁₋₃ alkyl, C₂₋₃ alkenyl, and C₂₋₃ alkynyl; or
R^{4B} and R^{4C}, together with the N atom to which they are both attached, optionally form a 4-, 5-, 6-, or 7-membered heterocycloalkyl group that is optionally substituted with 1, 2, or 3 substituents independently selected from independently selected from R^{4D};
each R^{4D} is independently selected from C₁₋₃ alkyl, C₁₋₃ haloalkyl, C₂₋₃ alkenyl, and C₂₋₃ alkynyl;
each R^{4E} is independently selected from D, halo, CN, OH, NH₂, NH(C₁₋₃ alkyl), and N(C₁₋₃ alkyl)₂;
X⁵ is N or CR⁵;
R⁵ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, halo, CN, OR^{5A}, and NR^{5B}R^{5C}; wherein the C₃₋₁₀ cycloalkyl and 4-10 membered heterocycloalkyl forming R⁵ are each optionally substituted with 1, 2, or 3 substituents independently selected from R^{5D}; and wherein the C₁₋₃ alkyl, C₂₋₃ alkenyl, and C₂₋₃ alkynyl forming R⁵ are each optionally substituted with 1, 2, or 3 substituents independently selected from R^{5E};
R^{5A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{5B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{5C} is selected from H, C₁₋₃ alkyl, C₂₋₃ alkenyl, and C₂₋₃ alkynyl; or
R^{5B} and R^{5C}, together with the N atom to which they are both attached, optionally form a 4-, 5-, 6-, or 7-membered heterocycloalkyl group that is optionally substituted with 1, 2, or 3 substituents independently selected from independently selected from R^{5D};
each R^{5D} is independently selected from C₁₋₃ alkyl, C₁₋₃ haloalkyl, C₂₋₃ alkenyl, and C₂₋₃ alkynyl;
each R^{5E} is independently selected from D, halo, CN, OH, NH₂, NH(C₁₋₃ alkyl), and N(C₁₋₃ alkyl)₂;
X⁶ is N or CR⁶;
R⁶ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, CN, OR^{6A}, and NR^{6B}R^{6C}; wherein the C₃₋₁₀ cycloalkyl, 4-10 membered heterocycloalkyl, C₆₋₁₀ aryl, and 5-10 membered heteroaryl forming R⁶ are each optionally substituted with 1, 2, or 3 substituents independently selected from R^{6D}; wherein the C₁₋₃ alkyl, C₂₋₃ alkenyl, and C₂₋₃ alkynyl forming R⁶ are each optionally substituted with 1, 2, or 3 substituents independently selected from R^{6E};
R^{6A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{6A} are each optionally substituted with 1, 2, or 3 substituents independently selected from R^{6D}; wherein the C₁₋₃ alkyl, C₂₋₃ alkenyl, and C₂₋₃ alkynyl forming R^{6A} are each optionally substituted with 1, 2, or 3 substituents independently selected from R^{6E};
R^{6B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{6B} are each optionally substituted with 1, 2, or 3 substituents independently selected from R^{6D}; wherein the C₁₋₃ alkyl, C₂₋₃ alkenyl, and C₂₋₃ alkynyl forming R^{6B} are each optionally substituted with 1, 2, or 3 substituents independently selected from R^{6E};
R^{6C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{6C} are each optionally substituted with 1, 2, or 3 substituents independently selected from R^{6E}; or
R^{6B} and R^{6C}, together with the N atom to which they are both attached, optionally form a 4-, 5-, 6-, or 7-membered heterocycloalkyl group that is optionally substituted with 1, 2, or 3 substituents independently selected from independently selected from R^{6D};
each R^{6D} is independently selected from C₁₋₃ alkyl, C₁₋₃ haloalkyl, C₂₋₃ alkenyl, C₂₋₃ alkynyl, and R^{6E}; wherein each of the C₁₋₃ alkyl, C₂₋₃ alkenyl, and C₂₋₃ alkynyl forming R^{6D} is optionally substituted with 1, 2, or 3 substituents independently selected from R^{6E};
each R^{6E} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a6}, R^{b6}, R^{c6}, and R^{d6} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6A}, C(═NR^{e6A})NR^{c6A}R^{d6A}, NR^{c6A}C(═NR^{e6A})NR^{c6A}R^{d6A}, S(O)R^{b6A}, S(O)NR^{c6A}R^{d6A}, S(O)₂R^{b6A}, NR^{c6A}S(O)₂R^{b6A}, and S(O)₂NR^{c6A}R^{d6A}; or
R^{c6} and R^{d6} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6A}, C(═NR^{e6A})NR^{c6A}R^{d6A}, NR^{c6A}C(═NR^{e6A})NR^{c6A}R^{d6A}, S(O)R^{b6A}, S(O)NR^{c6A}R^{d6A}, S(O)₂R^{b6A}, NR^{c6A}S(O)₂R^{b6A}, and S(O)₂NR^{c6A}R^{d6A};
R^{a6A}, R^{b6A}, R^{c6A}, and R^{d6A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a6A}, R^{b6A}, R^{c6A}, and R^{d6A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy; or
R^{c6A} and R^{d6A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;
R^{e6} and R^{e6A} are each, independently, H, CN or NO₂;
X⁷ is N or CR⁷;
R⁷ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, OR^{7A}, and NR^{7B}R^{7C}; wherein the C₃₋₁₀ cycloalkyl, 4-10 membered heterocycloalkyl, C₆₋₁₀ aryl, and 5-10 membered heteroaryl forming R⁷ are each optionally substituted with 1, 2, or 3 substituents independently selected from R^{7D}; wherein the C₁₋₃ alkyl, C₂₋₃ alkenyl, and C₂₋₃ alkynyl forming R⁷ are each optionally substituted with 1, 2, or 3 substituents independently selected from R^{7E};
R^{7A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{7A} are each optionally substituted with 1, 2, or 3 substituents independently selected from R^{7D}; wherein the C₁₋₃ alkyl, C₂₋₃ alkenyl, and C₂₋₃ alkynyl forming R^{7A} are each optionally substituted with 1, 2, or 3 substituents independently selected from R^{7E};
R^{7B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{7B} are each optionally substituted with 1, 2, or 3 substituents independently selected from R^{7D}; wherein the C₁₋₃ alkyl, C₂₋₃ alkenyl, and C₂₋₃ alkynyl forming R^{7B} are each optionally substituted with 1, 2, or 3 substituents independently selected from R^{7E};
R^{7C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{7C} are each optionally substituted with 1, 2, or 3 substituents independently selected from R^{7E}; or
R^{7B} and R^{7C}, together with the N atom to which they are both attached, optionally form a 4-, 5-, 6-, or 7-membered heterocycloalkyl group that is optionally substituted with 1, 2, or 3 substituents independently selected from independently selected from R^{7D};
each R^{7D} is independently selected from C₁₋₃ alkyl, C₁₋₃ haloalkyl, C₂₋₃ alkenyl, C₂₋₃ alkynyl, and R^{7E}; wherein each of the C₁₋₃ alkyl, C₂₋₃ alkenyl, and C₂₋₃ alkynyl forming R^{7D} is optionally substituted with 1, 2, or 3 substituents independently selected from R^{7E};
each R^{7E} is independently selected from D, halo, CN, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)NR^{c7}R^{d7}, NR^{c7}C(O)OR^{a7}, C(═NR^{e7})NR^{c7}R^{d7}, NR^{c7}C(═NR^{e7})NR^{c7}R^{d7}, S(O)R^{b7}, S(O)NR^{c7}R^{d7}, S(O)₂R^{b7}, NR^{c7}S(O)₂R^{b7}, and S(O)₂NR^{c7}R^{d7};
R^{a7}, R^{b7}, R^{c7}, and R^{d7} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a7}, R^{b7}, R^{c7}, and R^{d7} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a7A}, SR^{a7A}, C(O)R^{b7A}, C(O)NR^{7A}R^{d7A}, C(O)OR^{a7A}, OC(O)R^{b7A}, OC(O)NR^{c7A}R^{d7A}, NR^{c7A}R^{d7A}, NR^{c7A}C(O)R^{b7A}, NR^{c7A}C(O)NR^{c7A}R^{d7A}, NR^{c7A}C(O)OR^{a7A}, C(═NR^{e7A})NR^{c7A}R^{d7A}, NR^{c7A}C(═NR^{e7A})NR^{c7A}R^{d7A}, S(O)R^{b7A}, S(O)NR^{c7A}R^{d7A}, S(O)₂R^{b7A}, NR^{c7A}S(O)₂R^{b7A}, and S(O)₂NR^{c7A}R^{d7A}; or
R^{c7} and R^{d7} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a7A}, SR^{a7A}, C(O)R^{b7A}, C(O)NR^{c7A}R^{d7A}, C(O)OR^{a7A}, OC(O)R^{b7A}, OC(O)NR^{c7A}R^{d7A}, NR^{c7A}R^{d7A}, NR^{c7A}C(O)R^{b7A}, NR^{c7A}C(O)NR^{c7A}R^{d7A}, NR^{c7A}C(O)OR^{a7A}, C(═NR^{e7A})NR^{c7A}R^{d7A}, NR^{c7A}C(═NR^{e7A})NR^{c7A}R^{d7A}, S(O)R^{b7A}, S(O)NR^{c7A}R^{d7A}, S(O)₂R^{b7A}, NR^{c7A}S(O)₂R^{b7A}, and S(O)₂NR^{c7A}R^{d7A};
R^{a7A}, R^{b7A}, R^{c7A}, and R^{d7A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a7A}, R^{b7A}, R^{c7A}, and R^{d7A} are each optionally substituted with 1, 2, or 3 substituents independently selected from OH, CN, amino, NH(C₁₋₆ alkyl), N(C₁₋₆ alkyl)₂, halo, C₁₋₆alkyl, C₁₋₆ alkoxy, C₁₋₆ haloalkyl, and C₁₋₆ haloalkoxy; or
R^{c7A} and R^{d7A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;
R^{e7} and R^{e7A} are each, independently, H, CN or NO₂;
X⁸ is N or CRB;
R⁸ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, CN, OR^{8A}, and NR^{8B}R^{8C}; wherein the C₃₋₁₀ cycloalkyl, 4-10 membered heterocycloalkyl, C₆₋₁₀ aryl, and 5-10 membered heteroaryl forming R⁸ are each optionally substituted with 1, 2, or 3 substituents independently selected from R^{8D}; wherein the C₁₋₃ alkyl, C₂₋₃ alkenyl, and C₂₋₃ alkynyl forming R⁸ are each optionally substituted with 1, 2, or 3 substituents independently selected from R^{8E};
R^{8A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{8A} are each optionally substituted with 1, 2, or 3 substituents independently selected from R^{8D}; wherein the C₁₋₃ alkyl, C₂₋₃ alkenyl, and C₂₋₃ alkynyl forming R^{8A} are each optionally substituted with 1, 2, or 3 substituents independently selected from R^{8E};
R^{8B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{8B} are each optionally substituted with 1, 2, or 3 substituents independently selected from R^{8D}; wherein the C₁₋₃ alkyl, C₂₋₃ alkenyl, and C₂₋₃ alkynyl forming R^{8B} are each optionally substituted with 1, 2, or 3 substituents independently selected from R^{8E};
R^{8C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{8C} are each optionally substituted with 1, 2, or 3 substituents independently selected from R^{8E}; or
R^{8B} and R^{8C}, together with the N atom to which they are both attached, optionally form a 4-, 5-, 6-, or 7-membered heterocycloalkyl group that is optionally substituted with 1, 2, or 3 substituents independently selected from independently selected from R^{8D};
each R^{8D} is independently selected from C₁₋₃ alkyl, C₁₋₃ haloalkyl, C₂₋₃ alkenyl, C₂₋₃ alkynyl, and R^{8E}; wherein each of the C₁₋₃ alkyl, C₂₋₃ alkenyl, and C₂₋₃ alkynyl forming R^{8D} is optionally substituted with 1, 2, or 3 substituents independently selected from R^{8E};
each R^{8E} is independently selected from D, halo, CN, OR^{a8}, SR^{a8}, C(O)R^{b8}, C(O)NR^{c8}R^{d8}, C(O)OR^{a8}, OC(O)R^{b8}, OC(O)NR^{c8}R^{d8}, NR^{c8}R^{d8}, NR^{c8}C(O)R^{b8}, NR^{c8}C(O)NR^{c8}R^{d8}, NR^{c8}C(O)OR^{a8}, C(═NR^{e8})NR^{c8}R^{d8}, NR^{c8}C(═NR^{e8})NR^{c8}R^{d8}, S(O)R^{b8}, S(O)NR^{c8}R^{d8}, S(O)₂R^{b8}, NR^{c8}S(O)₂R^{b8}, and S(O)₂NR^{c8}R^{d8};
R^{a8}, R^{b8}, R^{c8}, and R^{d8} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a8}, R^{b8}, R^{c8}, and R^{d8} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a8A}, SR^{a8A}, C(O)R^{b8A}, C(O)NR^{c8A}R^{d8A}, C(O)OR^{a8A}, OC(O)R^{b8A}, OC(O)NR^{c8A}R^{d8A}, NR^{c8A}R^{d8A}, NR^{c8A}C(O)R^{b8A}, NR^{c8A}C(O)NR^{c8A}R^{d8A}, NR^{c8A}C(O)OR^{a8A}, C(═NR^{e8A})NR^{c8A}R^{d8A}, NR^{c8A}C(═NR^{e8A})NR^{c8A}R^{d8A}, S(O)R^{b8A}, S(O)NR^{c8A}R^{d8A}, S(O)₂R^{b8A}, NR^{c8A}S(O)₂R^{b8A}, and S(O)₂NR^{c8A}R^{d8A}; or
R^{c8} and R^{d8} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a8A}, SR^{a8A}, C(O)R^{b8A}, C(O)NR^{c8A}R^{d8A}, C(O)OR^{a8A}, OC(O)R^{b8A}, OC(O)NR^{c8A}R^{d8A}, NR^{c8A}R^{d8A}, NR^{c8A}C(O)R^{b8A}, NR^{c8A}C(O)NR^{c8A}R^{d8A}, NR^{c8A}C(O)OR^{a8A}, C(═NR^{e8A})NR^{c8A}R^{d8A}, NR^{c8A}C(═NR^{e8A})NR^{c8A}R^{d8A}, S(O)R^{b8A}, S(O)NR^{c8A}R^{d8A}, S(O)₂R^{b8A}, NR^{c8A}S(O)₂R^{b8A}, and S(O)₂NR^{c8A}R^{d8A};
R^{a8A}, R^{b8A}, R^{c8A}, and R^{d8A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a8A}, R^{b8A}, R^{c8A}, and R^{d8A} are each optionally substituted with 1, 2, or 3 substituents independently selected from OH, CN, amino, NH(C₁₋₆ alkyl), N(C₁₋₆ alkyl)₂, halo, C₁₋₆alkyl, C₁₋₆ alkoxy, C₁₋₆ haloalkyl, and C₁₋₆ haloalkoxy; or
R^{c8A} and R^{d8A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;
R^{e8} and R^{e8A} are each, independently, H, CN or NO₂;
X⁹ is N or CR⁹;
R⁹ is selected from H, C₁₋₃ alkyl, C₁₋₃ haloalkyl, C₂₋₃ alkenyl, C₂₋₃ alkynyl, CN, halo, OR^{9A}, and NR^{9B}R^{9C};
R^{9A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{9B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{9B} are each optionally substituted with 1, 2, or 3 substituents independently selected from R^{9D};
R^{9C} is selected from H, C₁₋₃ alkyl, C₂₋₃ alkenyl, and C₂₋₃ alkynyl;
each R^{9D} is independently selected from C₁₋₃ alkyl, C₁₋₃ haloalkyl, C₂₋₃ alkenyl, and C₂₋₃ alkynyl;
R¹⁰ is selected from H, C₁₋₃ alkyl, C₁₋₃ haloalkyl, C₂₋₃ alkenyl, C₂₋₃ alkynyl, CN, halo, OR^{10A}, and NR^{10B}R^{10C};
R^{10A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{10B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{10B} are each optionally substituted with 1, 2, or 3 substituents independently selected from R^{10D};
R^{10C} is selected from H, C₁₋₃ alkyl, C₂₋₃ alkenyl, and C₂₋₃ alkynyl;
each R^{10D} is independently selected from C₁₋₃ alkyl, C₁₋₃ haloalkyl, C₂₋₃ alkenyl, and C₂₋₃ alkynyl;
R¹¹ is selected from H, C₁₋₃ alkyl, C₁₋₃ haloalkyl, C₂₋₃ alkenyl, C₂₋₃ alkynyl, CN, halo, OR^{11A}, and NR^{11B}R^{11C};
R^{11A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{11B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{11B} are each optionally substituted with 1, 2, or 3 substituents independently selected from R^{11D};
R^{11C} is selected from H, C₁₋₃ alkyl, C₂₋₃ alkenyl, and C₂₋₃ alkynyl;
each R^{11D} is independently selected from C₁₋₃ alkyl, C₁₋₃ haloalkyl, C₂₋₃ alkenyl, and C₂₋₃ alkynyl;
X¹² is O or S;
R¹³ is independently selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{13A}, and NR^{13B}R^{13C};
R^{13A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{13B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{13B} are each optionally substituted with 1, 2, or 3 substituents independently selected from R^{13D};
R^{13C} is selected from H, C₁₋₃ alkyl, C₂₋₃ alkenyl, and C₂₋₃ alkynyl; each R^{13D} is independently selected from C₁₋₃ alkyl, C₁₋₃ haloalkyl, C₂₋₃ alkenyl, and C₂₋₃ alkynyl;
R¹⁴ is independently selected from C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{14A}, and NR^{14B}R^{14C};
R^{14A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{14B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{14B} are each optionally substituted with 1, 2, or 3 substituents independently selected from R^{14D};
R^{14C} is selected from H, C₁₋₃ alkyl, C₂₋₃ alkenyl, and C₂₋₃ alkynyl; and
each R^{c4D} is independently selected from C₁₋₃ alkyl, C₁₋₃ haloalkyl, C₂₋₃ alkenyl, and C₂₋₃ alkynyl;
wherein the ring-forming atoms of heterocycloalkyl and heteroaryl consist of at least one ring-forming carbon atom and 1, 2, or 3 heteroatoms independently selected from N, O, and S; and wherein a ring-forming carbon atom of the heterocycloalkyl and heteroaryl is optionally substituted by oxo to form a carbonyl group.

In an embodiment,
each R^{1B} is independently selected from D, C₃₋₆cycloalkyl, 4-10 membered heterocycloalkyl, phenyl, 5-6 membered heteroaryl, halo, CN, OR^{a1B}, C(O)R^{b1B}, C(O)NR^{c1B}R^{d1B}, C(O)OR^{a1B}, and NR^{c1B}R^{d1B}; wherein the C₃₋₆ cycloalkyl, 4-6 membered heterocycloalkyl, phenyl, and 5-6 membered heteroaryl forming R^{1B} are each optionally substituted with 1, 2, or 3 substituents independently selected from R^{1C};
R³ is selected from H, D, C₁₋₃ alkyl, C₁₋₃ haloalkyl, C₂₋₃ alkenyl, C₂₋₃ alkynyl, C₃₋₅ cycloalkyl, 4-8 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a3}, C(O)R^{b3}, C(O)NR^{c3}R^{d3}, NR^{c3}R^{e3}, and NR^{c3}C(O)R^{b3}; wherein the C₃₋₅ cycloalkyl, 4-8 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R³ are each optionally substituted with 1, 2, or 3 substituents independently selected from R^{3A}; wherein the C₁₋₃ alkyl, C₂₋₃ alkenyl, and C₂₋₃ alkynyl forming R³ are each optionally substituted with 1, 2, or 3 substituents independently selected from R^{3B}; and
each R^{3B} is independently selected from D, C₃₋₆ cycloalkyl, 4-10 membered heterocycloalkyl, phenyl, 5-6 membered heteroaryl, halo, CN, C(O)R^{b3B}, C(O)NR^{c3B}R^{d3B}, C(O)OR^{a3B}, NR^{c3B}R^{d3B}, and S(O)₂R^{b3B}; wherein the C₃₋₆cycloalkyl, 4-6 membered heterocycloalkyl, phenyl, and 5-6 membered heteroaryl forming R^{3B} are each optionally substituted with 1, 2, or 3 substituents independently selected from R^{3C}.

In an embodiment, the compound of Formula (I) is a compound of Formula (Ia), (Ib), (Ic), (Id), (Ie), or (If):

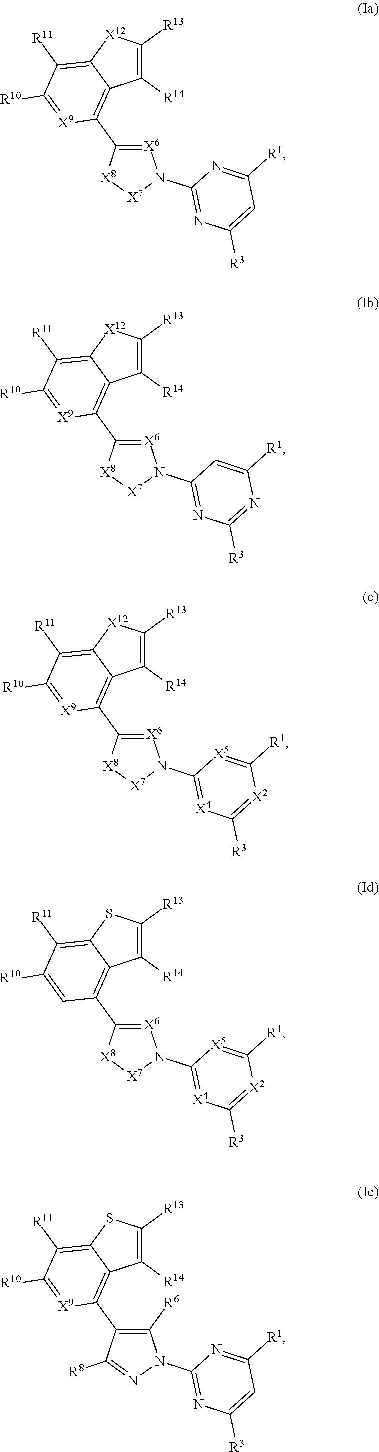

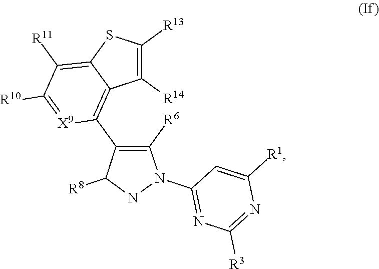

or a pharmaceutically acceptable salt thereof.

In another embodiment, the compound of Formula (I) is a compound of Formula (Ig), (Ih), (Ii), (Ij), (Ik), (Im), or (In):

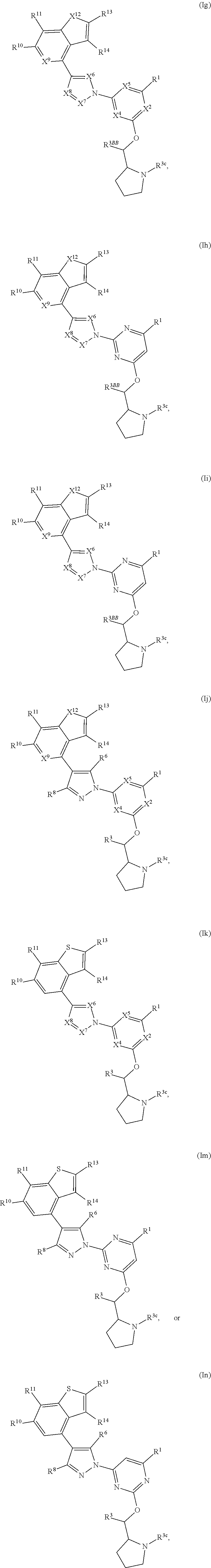

or a pharmaceutically acceptable salt thereof,
wherein R^{3BB} is H or C₁₋₃ alkyl. In an embodiment, R^{3BB} is C₁₋₃ alkyl. In another embodiment, R^{3BB} is methyl.

In an embodiment, the compound of Formula (I) is a compound of Formula (Io) or (Ip):

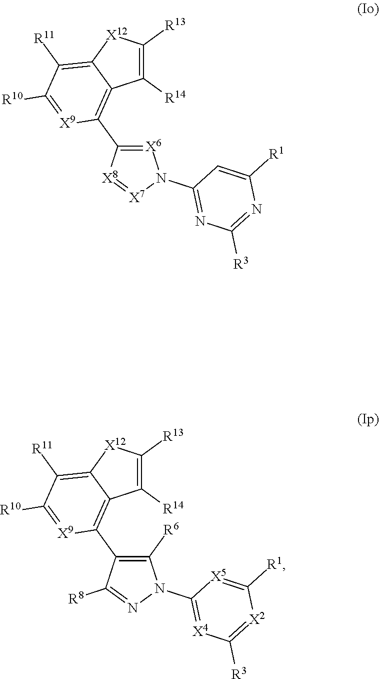

or a pharmaceutically acceptable salt thereof;
wherein.
R³ is 4-9 membered heterocycloalkyl optionally substituted with 1, 2, or 3 substituents independently selected from R^{3A};
each R^{3A} is independently selected from C₁₋₃ alkyl, C₁₋₃ haloalkyl, C₂₋₃ alkenyl, C₂₋₃ alkynyl, and R^{3B}; and
each R^{3B} is independently selected from D, C₃₋₆ cycloalkyl, 4-10 membered heterocycloalkyl, phenyl, 5-6 membered heteroaryl, halo, CN, and NR^{c3B}C(O)R^{b3B}.

In another embodiment,
R¹ is selected from H, D, C₁₋₃ alkyl, C₁₋₃ haloalkyl, C₂₋₃ alkenyl, C₂₋₃ alkynyl, C₃₋₅ cycloalkyl, 4-10 membered heterocycloalkyl, phenyl, 5-10 membered heteroaryl, halo, CN, OR^{a1}, C(O)R^{b1}, NR^{c1B}C(O)R^{b1B}, C(O)NR^{c1}R^{d1}, NR^{c1}R^{e1}, and NR^{c1}C(O)R^{b1}; wherein the C₃₋₅ cycloalkyl, 4-10 membered heterocycloalkyl, phenyl, and 5-10 membered heteroaryl forming R¹ are each optionally substituted with 1, 2, or 3 substituents independently selected from R^{1A};
each R^{1A} is independently selected from C₁₋₃ alkyl, C₁₋₃ haloalkyl, and R^{1B}; wherein the C₁₋₃ alkyl forming R^{1A} is optionally substituted with 1, 2, or 3 substituents each independently selected from R^{1B};
each R^{1B} is independently selected from D, C₃₋₆cycloalkyl, 4-10 membered heterocycloalkyl, phenyl, 5-6 membered heteroaryl, halo, CN, OR^{a1B}, C(O)R^{b1B}, C(O)NR^{c1B}R^{d1B}, C(O)OR^{a1B}, and NR^{c1B}R^{d1B}; wherein the C₃₋₆ cycloalkyl, 4-6 membered heterocycloalkyl, phenyl, and 5-6 membered heteroaryl forming R^{1B} are each optionally substituted with 1, 2, or 3 substituents independently selected from R^{1C};
each R^{1C} is independently selected from D, C₃₋₆ cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C}; wherein the C₃₋₆cycloalkyl, 4-6 membered heterocycloalkyl, phenyl, and 5-6 membered heteroaryl forming R^{1C} are each optionally substituted with 1, 2, or 3 substituents independently selected from R^{1D};
each R^{1D} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a1D}, C(O)R^{b1D}, C(O)NR^{c1D}R^{d1D}, C(O)OR^{a1D}, NR^{c1D}R^{d1D}, and S(O)₂R^{b1D};
each R^{a1} is independently selected from C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a1} are each optionally substituted with 1, 2, or 3 substituents independently selected from R^{1A};
each R^{b1}, R^{c1}, and R^{d1} is independently selected from H, C₁₋₃ alkyl, C₁₋₃ haloalkyl, C₂₋₃ alkenyl, and C₂₋₃ alkynyl;
each R^{e1} is independently selected from C₁₋₃ alkyl, C₁₋₃ haloalkyl, C₂₋₃ alkenyl, and C₂₋₃ alkynyl; wherein the C₁₋₃ alkyl, C₁₋₃ haloalkyl, C₂₋₃ alkenyl, and C₂₋₃ alkynyl forming R^{e1} are each optionally substituted with 1, 2, or 3 substituents independently selected from R^{1B};
each R^{a1B}, R^{b1B}, R^{c1B} and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1C}, R^{b1C}, R^{c1C}, and R^{d1C} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1D}, R^{b1D}, R^{c1D}, and R^{d1D} is independently selected from H, C₁₋₃ alkyl, and C₁₋₃ haloalkyl;
X² is N or CH;
R³ is selected from H, D, C₁₋₃ alkyl, C₁₋₃ haloalkyl, C₂₋₃ alkenyl, C₂₋₃ alkynyl, C₃₋₅ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a3}, C(O)R^{b3}, C(O)NR^{c3}R^{d3}, NR^{c3}R^{e3}, and NR^{c3}C(O)R^{b3}, wherein 4-9 membered heterocycloalkyl is optionally substituted with R^{3A};
each R^{3A} is independently selected from C₁₋₃ alkyl, C₁₋₃ haloalkyl, C₂₋₃ alkenyl, C₂₋₃ alkynyl, and R^{3B};
each R^{3B} is independently selected from D, C₃₋₆ cycloalkyl, 4-10 membered heterocycloalkyl, phenyl, 5-6 membered heteroaryl, halo, CN, and NR^{c3B}C(O)R^{b3B}; wherein the C₃₋₆ cycloalkyl, 4-6 membered heterocycloalkyl, phenyl, and 5-6 membered heteroaryl forming R^{3B} are each optionally substituted with 1, 2, or 3 substituents independently selected from R^{3C};
each R^{3C} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, and CN;
each R^{a3} is independently selected from C₁₋₆ alkyl, C₁₋₃ haloalkyl, C₂₋₆ alkenyl, C₂₋₆ alkynyl, C₃₋₆ cycloalkyl, 4-6 membered heterocycloalkyl, phenyl, and 5-6 membered heteroaryl; wherein the C₁₋₆ alkyl, C₂₋₆ alkenyl, and C₂₋₆ alkynyl forming R^{a3} are each optionally substituted with 1, 2, or 3 substituents independently selected from R^{3B};
X⁴ is N;
X⁵ is N or CR⁵;
R⁵ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, halo, CN, OR^{5A}, and NR^{5B}R^{5C};
R^{5A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{5B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{5C} is selected from H, C₁₋₃ alkyl, C₂₋₃ alkenyl, and C₂₋₃ alkynyl;
X⁶ is CR⁶;
R⁶ is selected from C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, CN, OR^{6A}, and NR^{6B}R^{6C};
R^{6A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{6B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{6C} is selected from H, C₁₋₃ alkyl, C₂₋₃ alkenyl, and C₂₋₃ alkynyl;
X⁷ is N, CH, of C-halo;
X⁸ is CR⁸;
R⁸ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, CN, OR^{8A}, and NR^{8B}R^{8C};
R^{8A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{8B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{8C} is selected from H, C₁₋₃ alkyl, C₂₋₃ alkenyl, and C₂₋₃ alkynyl;
X⁹ is N or CH;
R¹⁰ is selected from H, C₁₋₃ alkyl, C₁₋₃ haloalkyl, C₂₋₃ alkenyl, C₂₋₃ alkynyl, CN, halo, OR^{10A}, and NR^{10B}R^{10C};
R^{10A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{10B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{10B} are each optionally substituted with 1, 2, or 3 substituents independently selected from R^{10D};
R^{10C} is selected from H, C₁₋₃ alkyl, C₂₋₃ alkenyl, and C₂₋₃ alkynyl;
each R^{10D} is independently selected from C₁₋₃ alkyl, C₁₋₃ haloalkyl, C₂₋₃ alkenyl, and C₂₋₃ alkynyl;
R¹¹ is selected from H, C₁₋₃ alkyl, C₁₋₃ haloalkyl, C₂₋₃ alkenyl, C₂₋₃ alkynyl, CN, halo, OR^{11A}, and NR^{11B}R^{11C};
R^{11A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{11B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{11B} are each optionally substituted with 1, 2, or 3 substituents independently selected from R^{11D};
R^{11C} is selected from H, C₁₋₃ alkyl, C₂₋₃ alkenyl, and C₂₋₃ alkynyl; each R^{11D} is independently selected from C₁₋₃ alkyl, C₁₋₃ haloalkyl, C₂₋₃ alkenyl, and C₂₋₃ alkynyl;
X¹² is O or S;
R¹³ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{13A}, and NR^{13B}R^{13C};
R^{13A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{13B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{13C} is selected from H, C₁₋₃ alkyl, C₂₋₃ alkenyl, and C₂₋₃ alkynyl;
R¹⁴ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{14A}, and NR^{14B}R^{14C};
R^{14A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{14B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; and
R^{14C} is selected from H, C₁₋₃ alkyl, C₂₋₃ alkenyl, and C₂₋₃ alkynyl.

In an embodiment,
R¹ is selected from H, D, C₁₋₃ alkyl, C₁₋₃ haloalkyl, C₂₋₃ alkenyl, C₂₋₃ alkynyl, C₃₋₅ cycloalkyl, 4-10 membered heterocycloalkyl, phenyl, 5-10 membered heteroaryl, halo, CN, OR^{a1}, C(O)R^{b1}, C(O)NR^{c1}R^{d1}, NR^{c1}R^{e1}, and NR^{c1}C(O)R^{b1}; wherein the C₃₋₅ cycloalkyl, 4-10 membered heterocycloalkyl, phenyl, and 5-10 membered heteroaryl forming R¹ are each optionally substituted with 1, 2, or 3 substituents independently selected from R^{1A};
R³ is selected from H, D, C₁₋₃ alkyl, C₁₋₃ haloalkyl, C₂₋₃ alkenyl, C₂₋₃ alkynyl, C₃₋₅ cycloalkyl, 4-8 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a3}, C(O)R^{b3}, C(O)NR^{c3}R^{d3}, NR^{c3}R^{e3}, and NR^{c3}C(O)R^{b3};
each R^{3B} is independently selected from D, C₃₋₆ cycloalkyl, 4-10 membered heterocycloalkyl, phenyl, 5-6 membered heteroaryl, halo, and CN; wherein the C₃₋₆ cycloalkyl, 4-6 membered heterocycloalkyl, phenyl, and 5-6 membered heteroaryl forming R^{3B} are each optionally substituted with 1, 2, or 3 substituents independently selected from R^{3C}.

In another embodiment,
R¹ is selected from C₃₋₅ cycloalkyl, 4-10 membered heterocycloalkyl, phenyl, 5-10 membered heteroaryl, halo, CN, OR^{a1}, C(O)R^{b1}, C(O)NR^{c1}R^{d1}, NR^{c1}R^{e1}, and NR^{c1}C(O)R^{b1}; wherein the C₃₋₅ cycloalkyl, 4-10 membered heterocycloalkyl, phenyl, and 5-10 membered heteroaryl forming R¹ are each optionally substituted with 1 or 2 substituents independently selected from R^{1A}.
each R^{1A} is independently selected from C₁₋₃ alkyl, C₁₋₃ haloalkyl, and R^{1B}; wherein the C₁₋₃ alkyl forming R^{1A} is optionally substituted with 1 or 2 substituents each independently selected from R^{1B};
each R^{1B} is independently selected from C₃₋₆ cycloalkyl, 4-10 membered heterocycloalkyl, phenyl, 5-6 membered heteroaryl, halo, CN, OR^{a1B}, C(O)R^{b1B}, NR^{c1B}C(O)R^{b1B}, C(O)NR^{c1B}R^{d1B}, C(O)OR^{a1B}, and NR^{c1B}R^{d1B}; wherein the C₃₋₆cycloalkyl, 4-6 membered heterocycloalkyl, phenyl, and 5-6 membered heteroaryl forming R^{1B} are each optionally substituted with 1 or 2 substituents independently selected from R^{1C};
each R^{1C} is independently selected from D, C₃₋₆ cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C}; wherein the C₃₋₆cycloalkyl, 4-6 membered heterocycloalkyl, phenyl, and 5-6 membered heteroaryl forming R^{1C} are each optionally substituted with 1, 2, or 3 substituents independently selected from R^{1D};
each R^{1D} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a1D}, C(O)R^{b1D}, C(O)NR^{c1D}R^{d1D}, C(O)OR^{a1D}, NR^{c1D}R^{d1D}, and S(O)₂R^{b1D};
each R^{a1} is independently selected from C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a1} are each optionally substituted with 1 or 2 substituents independently selected from R^{1A};
each R^{b1}, R^{c1}, and R^{d1} is independently selected from H and C₁₋₃ alkyl;
each R^{e1} is independently selected from C₁₋₃ alkyl; wherein the C₁₋₃ alkyl forming R^{e1} is optionally substituted with 1 or 2 substituents independently selected from R^{1B};
each R^{a1B}, R^{b1B}, R^{c1B} and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1C}, R^{b1C}, R^{c1C}, and R^{d1C} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1D}, R^{b1D}, R^{c1D}, and R^{d1D} is independently selected from H, C₁₋₃ alkyl, and C₁₋₃ haloalkyl;
X² is N or CH;
R³ is selected from C₃₋₅ cycloalkyl, 4-9 membered heterocycloalkyl, phenyl, 5-6 membered heteroaryl, halo, CN, and OR^{a3}, wherein 4-9 membered heterocycloalkyl is optionally substituted with R^{3A};
each R^{3A} is independently selected from C₁₋₃ alkyl, C₁₋₃ haloalkyl, C₂₋₃ alkenyl, C₂₋₃ alkynyl, and R^{3B};
each R^{3B} is independently selected from C₃₋₆ cycloalkyl, 4-10 membered heterocycloalkyl, phenyl, 5-6 membered heteroaryl, and NR^{c3B}C(O)R^{b3B}; wherein the C₃₋₆ cycloalkyl, 4-6 membered heterocycloalkyl, phenyl, and 5-6 membered heteroaryl forming R^{3B} are each optionally substituted with 1 or 2 substituents independently selected from R^{3C};
each R^{3C} is independently selected from C₁₋₃ alkyl, C₁₋₃ haloalkyl, halo, D, and CN; each R^{a3} is independently selected from C₁₋₆ alkyl and C₁₋₃ haloalkyl; wherein the C₁₋₆ alkyl forming R^{a3} are each optionally substituted with 1 or 2 substituents independently selected from R^{3B};
X⁴ is N;
X⁵ is N or CR⁵;
R⁵ is selected from H, C₁₋₃ alkyl, C₁₋₃ haloalkyl, C₂₋₃ alkenyl, C₂₋₃ alkynyl, halo, CN, OR^{5A}, and NR^{5B}R^{5C};
R^{5A} is selected from C₁₋₃ alkyl, C₂₋₃ alkenyl, and C₂₋₃ alkynyl;
R^{5B} is selected from H and C₁₋₃ alkyl;
R^{5C} is selected from H and C₁₋₃ alkyl;
X⁶ is CR⁶;
R⁶ is selected from C₁₋₃ alkyl, C₁₋₃ haloalkyl, C₂₋₃ alkenyl, C₂₋₃ alkynyl, halo, and CN;
X⁷ is N or CH;
X⁸ is CR⁸;
R⁸ is selected from H, C₁₋₃ alkyl, C₁₋₃ haloalkyl, C₂₋₃ alkenyl, C₂₋₃ alkynyl, halo, and CN;
X⁹ is N or CH;
R¹⁰ is selected from H, C₁₋₃ alkyl, C₁₋₃ haloalkyl, CN, and halo;
R¹¹ is selected from H, C₁₋₃ alkyl, C₁₋₃ haloalkyl, CN, and halo;
X¹² is O or S;
R¹³ is selected from H, C₁₋₃ alkyl, C₁₋₃ haloalkyl, C₂₋₃ alkenyl, C₂₋₃ alkynyl, CN, halo, OR^{13A}, and NR^{13B}R^{13C};
R^{13A} is selected from H and C₁₋₃ alkyl;
R^{13B} is selected from H and C₁₋₃ alkyl;
R^{13C} is selected from H and C₁₋₃ alkyl;
R¹⁴ is selected from H, C₁₋₃ alkyl, C₁₋₃ haloalkyl, C₂₋₃ alkenyl, C₂₋₃ alkynyl, CN, halo, OR^{14A}, and NR^{14B}R^{14C};
R^{14A} is selected from H and C₁₋₃ alkyl;
R^{14B} is selected from H and C₁₋₃ alkyl; and
R^{14C} is selected from H and C₁₋₃ alkyl.

In an embodiment,
each R^{1B} is independently selected from C₃₋₆ cycloalkyl, 4-10 membered heterocycloalkyl, phenyl, 5-6 membered heteroaryl, halo, CN, OR^{a1B}, C(O)R^{b1B}, NR^{c1B}C(O)R^{b1B}, C(O)NR^{c1B}R^{d1B}, C(O)OR^{a1B}, and NR^{c1B}R^{d1B}; wherein the C₃₋₆cycloalkyl, 4-6 membered heterocycloalkyl, phenyl, and 5-6 membered heteroaryl forming R^{1B} are each optionally substituted with 1 or 2 substituents independently selected from R^{1C};
R³ is selected from C₃₋₅ cycloalkyl, 4-8 membered heterocycloalkyl, phenyl, 5-6 membered heteroaryl, halo, CN, and OR^{a3};
each R^{3B} is independently selected from C₃₋₆ cycloalkyl, 4-10 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{3B} are each optionally substituted with 1 or 2 substituents independently selected from R^{3C}.

In another embodiment,
R¹ is selected from C₃₋₅ cycloalkyl, 4-10 membered heterocycloalkyl, phenyl, 5-10 membered heteroaryl, halo, OR^{a1}, and NR^{c1}R^{e1}; wherein the C₃₋₅ cycloalkyl, 4-10 membered heterocycloalkyl, phenyl, and 5-10 membered heteroaryl forming R¹ are each optionally substituted with 1 or 2 substituents independently selected from R^{1A};
each R^{1A} is independently selected from C₁₋₃ alkyl, C₁₋₃ haloalkyl, and R^{1B}; wherein the C₁₋₃ alkyl forming R^{1A} is optionally substituted with 1 or 2 substituents each independently selected from R^{1B};
each R^{1B} is independently selected from C₃₋₆ cycloalkyl, 4-10 membered heterocycloalkyl, phenyl, 5-6 membered heteroaryl, halo, OR^{a1B}, C(O)R^{b1B}, NR^{c1B}C(O)R^{b1B}, and C(O)NR^{c1B}R^{d1B}; wherein the C₃₋₆ cycloalkyl, 4-6 membered heterocycloalkyl, phenyl, and 5-6 membered heteroaryl forming R^{1B} are each optionally substituted with 1 or 2 substituents independently selected from R^{1C};
each R^{1C} is independently selected from D, C₃₋₆ cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C};
each R^{a1} is independently selected from C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a1} are each optionally substituted with 1 or 2 substituents independently selected from R^{1A};
R^{c1} is selected from H and C₁₋₃ alkyl;
R^{e1} is selected from C₁₋₃ alkyl; wherein the C₁₋₃ alkyl forming R^{e1} is optionally substituted with 1 or 2 substituents independently selected from R^{1B};
each R^{a1B}, R^{b1B}, R^{c1B} and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl; and
each R^{a1C}, R^{b1C}, R^{c1C}, and R^{d1C} is independently selected from H and C₁₋₃ alkyl.

In an embodiment, each R^{1B} is independently selected from C₃₋₆cycloalkyl, 4-10 membered heterocycloalkyl, phenyl, 5-6 membered heteroaryl, halo, OR^{a1B}, C(O)R^{b1B}, and C(O)NR^{c1B}R^{d1B}; wherein the C₃₋₆ cycloalkyl, 4-6 membered heterocycloalkyl, phenyl, and 5-6 membered heteroaryl forming R^{1B} are each optionally substituted with 1 or 2 substituents independently selected from R^{1C}.

In another embodiment,
R¹ is selected from 4-10 membered heterocycloalkyl, 5-10 membered heteroaryl, halo, OR^{a1}, and NR^{c1}R^{e1}; wherein the 4-10 membered heterocycloalkyl and 5-10 membered heteroaryl forming R¹ are each optionally substituted with 1 or 2 substituents independently selected from R^{1A};
each R^{1A} is independently selected from C₁₋₃ alkyl, C₁₋₃ haloalkyl, and R^{1B}; wherein the C₁₋₃ alkyl forming R^{1A} is optionally substituted with 1 or 2 substituents each independently selected from R^{1B};
each R^{1B} is independently selected from 5-6 membered heteroaryl, halo,OR^{a1B}, C(O)R^{b1B}, NR^{c1B}C(O)R^{b1B}, and C(O)NR^{c1B}R^{d1B}; wherein the 5-6 membered heteroaryl forming R^{1B} is optionally substituted with 1 or 2 substituents independently selected from R^{1C};
each R^{1C} is independently selected from D, C₃₋₆ cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C};
each R^{a1} is independently selected from 4-6 membered heterocycloalkyl and 5-6 membered heteroaryl; wherein the 4-6 membered heterocycloalkyl and 5-6 membered heteroaryl forming R^{a1} are each optionally substituted with 1 or 2 substituents independently selected from R^{1A};
R^{c1} is H;
R^{e1} is C₁₋₃ alkyl optionally substituted with 1 or 2 substituents independently selected from R^{1B};
each R^{a1B}, R^{b1B}, R^{c1B} and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1C}, R^{b1C}, R^{c1C}, and R^{d1C} is independently selected from H and C₁₋₃ alkyl.

In an embodiment, each R^{1B} is independently selected from 5-6 membered heteroaryl, halo,OR^{a1B}, C(O)R^{b1B}, and C(O)NR^{c1B}R^{d1B}; wherein the 5-6 membered heteroaryl forming R^{1B} is optionally substituted with 1 or 2 substituents independently selected from R^{1C}.

In another embodiment, R¹ is selected from 4-acetylpiperazin-1-yl, N-methylpyrrolidine-3-carboxamide, 4-methyl-3-oxopiperazin-1-yl, 4-hydroxy-4-methylpiperidin-1-yl, N,N-dimethylpiperazine-1-carboxamide, 6-acetyl-2,6-diazaspiro[3.3]heptan-2-yl, 4-(cyclopropanecarbonyl)piperazin-1-yl, methylpiperazine-1-carboxylate, 2-methyl-3-oxohexahydroimidazo[1,5-a]pyrazin-7(1H)-yl, 6-acetyl-2,6-diazaspiro[3.4]octan-2-yl, 3-acetyl-3,8-diazabicyclo[3.2.1]octan-8-yl, 4-acetyl-3-(cyanomethyl)piperazin-1-yl, 5,6-dihydroimidazo[1,5-a]pyrazin-7(8H)-yl, 5,6-dihydro-[1,2,4]triazolo[4,3-a]pyrazin-7(8H)-yl, 4-(methylsulfonyl)piperazin-1-yl, 6-oxohexahydropyrrolo[1,2-a]pyrazin-2(1H)-yl, 5,6-dihydro-[1,2,4]triazolo[1,5-a]pyrazin-7(8H)-yl, pyridin-3-ylmethyl)amino)pyrimidin-2-yl, 1-(pyridin-3-yl)ethylamino, 1-acetylpiperidin-4-yl)amino, 1-methyl-1H-pyrazol-4-yl, 1-methyl-1H-pyrazol-3-yl, 3-methylisoxazol-5-yl, 1-methyl-6-oxo-1,6-dihydropyridin-3-yl, pyridin-3-yl, 2-hydroxypropan-2-yl)pyridin-3-yl, N,N-dimethylpicolinamide, N-methylpicolinamide, pyrimidin-5-yl, pyridin-4-yl, 1H-pyrazole-3-carboxamide, N-methyl-1H-pyrazole-3-carboxamide, N,N-dimethyl-1H-pyrazole-3-carboxamide, pyrimidin-5-yloxy, 1-acetylpiperidin-4-yloxy, 1-(oxetan-3-yl)-1H-pyrazol-4-yl, 1-(difluoromethyl)-1H-pyrazol-4-yl, 1-(pyridin-3-yl)-1H-pyrazol-4-yl, 1-benzyl-1H-pyrazol-4-yl, 1-(pyridin-3-ylmethyl)-1H-pyrazol-4-yl, 5-fluoro-1-methyl-1H-pyrazol-4-yl, 1,3-dimethyl-1H-pyrazol-4-yl, 1-(cyanomethyl)-1H-pyrazol-4-yl, N,N-dimethyl-5,6,7,8-tetrahydro-4H-pyrazolo[1,5-a][1,4]diazepine-2-carboxamide, and 4-(cyclopropanecarbonyl)piperazin-1-yl.

In another embodiment, X² is N. In another embodiment, X² is CH.

In another embodiment,
R³ is selected from C₃₋₅ cycloalkyl, 4-9 membered heterocycloalkyl, halo, and OR^{a3}, wherein 4-9 membered heterocycloalkyl is optionally substituted with R^{3A};
each R^{3A} is independently selected from C₁₋₃ alkyl, C₁₋₃ haloalkyl, C₂₋₃ alkenyl, C₂₋₃ alkynyl, and R^{3B};
each R^{3B} is independently selected from C₃₋₆ cycloalkyl, 4-10 membered heterocycloalkyl, and NR^{c3B}C(O)R^{b3B}; wherein the C₃₋₆cycloalkyl and 4-6 membered heterocycloalkyl forming R^{3B} are each optionally substituted with 1 or 2 substituents independently selected from R^{3C};
each R^{3C} is independently selected from C₁₋₃ alkyl, C₁₋₃ haloalkyl, and halo; and each R^{a3} is independently selected from C₁₋₆ alkyl and C₁₋₃ haloalkyl; wherein the C₁₋₆ alkyl forming R^{a3} are each optionally substituted with 1 or 2 substituents independently selected from R^{3B}.

In an embodiment,
R³ is selected from C₃₋₅ cycloalkyl, 4-8 membered heterocycloalkyl, halo, and OR^{a3}; and
each R^{3B} is independently selected from C₃₋₆ cycloalkyl, and 4-10 membered heterocycloalkyl; wherein the C₃₋₆ cycloalkyl and 4-6 membered heterocycloalkyl forming R^{3B} are each optionally substituted with 1 or 2 substituents independently selected from R^{3C}.

In another embodiment,
R³ is OR^{a3};
each R^{3B} is independently 4-10 membered heterocycloalkyl optionally substituted with 1 or 2 substituents independently selected from R^{3C};
each R^{3C} is independently C₁₋₃ alkyl; and
each R^{a3} is independently C₁₋₆ alkyl optionally substituted with 1 or 2 substituents independently selected from R^{3B}.

In another embodiment, R³ is OR^{a3}.

In another embodiment, each R^{3B} is 4-10 membered heterocycloalkyl optionally substituted with 1 or 2 substituents independently selected from R^{3C}.

In another embodiment, each R^{3C} is independently C₁₋₃ alkyl.

In another embodiment, each R^{a3} is independently selected from C₁₋₆ alkyl optionally substituted with 1 or 2 substituents independently selected from R^{3B}.

In another embodiment, X⁴ is N. In another embodiment, X⁴ is CR⁴. In another embodiment, X⁴ is CH.

In another embodiment, X⁵ is N. In another embodiment, X⁵ is CR⁵. In another embodiment, X⁵ is CH.

In another embodiment, R⁵ is selected from H, C₁₋₃ alkyl, C₁₋₃ haloalkyl, halo, and CN. In another embodiment, R⁵ is H, halo, or CN.

In another embodiment, X⁶ is N. In another embodiment, X⁶ is CR⁶. In another embodiment, X⁶ is CR⁶; and R⁶ is C₁₋₃ alkyl, C₁₋₃ haloalkyl, halo, or CN.

In another embodiment, R⁶ is C₁₋₃ alkyl, C₁₋₃ haloalkyl, or CN. In another embodiment, R⁶ is C₁₋₃ alkyl. In another embodiment, R⁶ is C₁₋₃ haloalkyl. In another embodiment, R⁶ is CN. In another embodiment, R⁶ is halo.

In another embodiment, X⁷ is N. In another embodiment, X⁷ is CR⁷. In another embodiment, X⁷ is CH. In another embodiment, X⁷ is C-halo. In another embodiment, R⁷ is H. In another embodiment, R⁷ is halo.

In another embodiment, X⁸ is N. In another embodiment, X⁸ is CR⁸. In another embodiment, X⁸ is CH. In another embodiment, X⁸ is C-halo. In another embodiment, X⁸ is CR⁸; and R⁸ is C₁₋₃ alkyl, C₁₋₃ haloalkyl, halo, or CN.

In another embodiment, R⁸ is C₁₋₃ alkyl, C₁₋₃ haloalkyl, halo, or CN. In another embodiment, R⁸ is C₁₋₃ alkyl. In another embodiment, R⁸ is C₁₋₃ haloalkyl. In another embodiment, R⁸ is CN. In another embodiment, R⁸ is halo.

In another embodiment, X⁹ is N. In another embodiment, X⁹ is CR⁹. In another embodiment, X⁹ is CH.

In another embodiment, R¹⁰ is H or halo. In another embodiment, R¹⁰ is H. In another embodiment, R¹⁰ is halo.

In another embodiment, R¹¹ is H or halo. In another embodiment, R¹¹ is H. In another embodiment, R¹¹ is halo.

In another embodiment, X¹² is S. In another embodiment, X¹² is O.

In another embodiment, R¹³ is selected from H, C₁₋₃ alkyl, C₁₋₃ haloalkyl, C₂₋₃ alkenyl, C₂₋₃ alkynyl, CN, halo, OR^{13A}, and NR^{13B}R^{13C}.

In another embodiment, R^{13B} is H; and R^{13C} is H.

In another embodiment, R¹³ is selected from H, C₁₋₃ alkyl, C₁₋₃ haloalkyl, CN, halo, OH, and NH₂.

In another embodiment, R¹⁴ is selected from H, C₁₋₃ alkyl, C₁₋₃ haloalkyl, C₂₋₃ alkenyl, C₂₋₃ alkynyl, CN, halo, OR^{14A}, and NR^{14B}R^{14C}.

In another embodiment, R^{14B} is H; and R^{14C} is H.

In another embodiment, R¹⁴ is selected from H, C₁₋₃ alkyl, C₁₋₃ haloalkyl, CN, halo, OH, and NH₂.

In other embodiments, the compound of Formula (I) is selected from a compound in Table 1, and pharmaceutically acceptable salts thereof.

TABLE 1

| Entry | Compound Name |
|---|---|
| 1 | 4-(1-(4-(4-acetylpiperazin-1-yl)-6-(1-(1-methylpyrrolidin-2-yl)ethoxy)pyrimidin-2-yl)- |
| | 3,5-dimethyl-1H-pyrazol-4-yl)-2-amino-7-fluorobenzo[b]thiophene-3-carbonitrile |
| 2 | 1-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N-methylpyrrolidine-3- |
| | carboxamide |
| 3 | 2-amino-4-(3,5-dimethyl-1-(4-(4-methyl-3-oxopiperazin-1-yl)-6-(1-(1- |
| | methylpyrrolidin-2-yl)ethoxy)pyrimidin-2-yl)-1H-pyrazol-4-yl)-7- |
| | fluorobenzo[b]thiophene-3-carbonitrile |
| 4 | 2-amino-7-fluoro-4-(1-(4-(4-hydroxy-4-methylpiperidin-1-yl)-6-(1-(1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 5 | 4-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N, N-dimethylpiperazine-1- |
| | carboxamide |
| 6 | 4-(1-(4-(6-acetyl-2,6-diazaspiro[3.3]heptan-2-yl)-6-(1-(1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 7 | 2-amino-4-(1-(4-(4-(cyclopropanecarbonyl)piperazin-1-yl)-6-(1-(1-methylpyrrolidin- |
| | 2-yl)ethoxy)pyrimidin-2-yl)-3,5-dimethyl-1H-pyrazol-4-yl)-7- |
| | fluorobenzo[b]thiophene-3-carbonitrile |
| 8 | methyl 4-(2-(4-(2-amino-3-cyano-7-fluorobenzo[b]thiophen-4-yl)-3,5-dimethyl-1H- |
| | pyrazol-1-yl)-6-(1-(1-methylpyrrolidin-2-yl)ethoxy)pyrimidin-4-yl)piperazine-1- |
| | carboxylate |
| 9 | 2-amino-4-(3,5-dimethyl-1-(4-(2-methyl-3-oxohexahydroimidazo[1,5-a]pyrazin- |
| | 7(1H)-yl)-6-(1-(1-methylpyrrolidin-2-yl)ethoxy)pyrimidin-2-yl)-1H-pyrazol-4-yl)-7- |
| | fluorobenzo[b]thiophene-3-carbonitrile |
| 10 | 4-(1-(4-(6-acetyl-2,6-diazaspiro[3.4]octan-2-yl)-6-(1-(1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 11 | 4-(1-(4-(3-acetyl-3,8-diazabicyclo[3.2.1]octan-8-yl)-6-(1-(1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 12 | 4-(1-(4-(4-acetyl-3-(cyanomethyl)piperazin-1-yl)-6-(1-(1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 13 | 2-amino-4-(1-(4-(5,6-dihydroimidazo[1,5-a]pyrazin-7(8H)-yl)-6-(1-(1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 14 | 2-amino-4-(1-(4-(5,6-dihydro-[1,2,4]triazolo[4,3-a]pyrazin-7(8H)-yl)-6-(1-(1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 15 | 2-amino-4-(3,5-dimethyl-1-(4-(1-(1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 16 | 2-amino-4-(3,5-dimethyl-1-(4-(1-(1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 17 | 2-amino-4-(1-(4-(5,6-dihydro-[1,2,4]triazolo[1,5-a]pyrazin-7(8H)-yl)-6-(1-(1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 18 | 2-amino-4-(3,5-dimethyl-1-(4-(1-(1-methylpyrrolidin-2-yl)ethoxy)-6-((pyridin-3- |
| | ylmethyl)amino)pyrimidin-2-yl)-1H-pyrazol-4-yl)-7-fluorobenzo[b]thiophene-3- |
| | carbonitrile |
| 19 | 2-amino-4-(3,5-dimethyl-1-(4-(1-(1-methylpyrrolidin-2-yl)ethoxy)-6-((1-(pyridin-3- |
| | yl)ethyl)amino)pyrimidin-2-yl)-1H-pyrazol-4-yl)-7-fluorobenzo[b]thiophene-3- |
| | carbonitrile |
| 20 | 4-(1-(4-((1-acetylpiperidin-4-yl)amino)-6-(1-(1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 21 | 2-amino-4-(3,5-dimethyl-1-(4-(1-methyl-1H-pyrazol-4-yl)-6-(1-(1-methylpyrrolidin- |
| | 2-yl)ethoxy)pyrimidin-2-yl)-1H-pyrazol-4-yl)-7-fluorobenzo[b]thiophene-3- |
| | carbonitrile |
| 22 | 2-amino-4-(3,5-dimethyl-1-(4-(1-methyl-1H-pyrazol-3-yl)-6-(1-(1-methylpyrrolidin- |
| | 2-yl)ethoxy)pyrimidin-2-yl)-1H-pyrazol-4-yl)-7-fluorobenzo[b]thiophene-3- |
| | carbonitrile |
| 23 | 2-amino-4-(3,5-dimethyl-1-(4-(3-methylisoxazol-5-yl)-6-(1-(1-methylpyrrolidin-2- |
| | yl)ethoxy)pyrimidin-2-yl)-1H-pyrazol-4-yl)-7-fluorobenzo[b]thiophene-3-carbonitrile |
| 24 | 2-amino-4-(3,5-dimethyl-1-(4-(1-methyl-6-oxo-1,6-dihydropyridin-3-yl)-6-(1-(1- |
| | methylpyrrolidin-2-yl)ethoxy)pyrimidin-2-yl)-1H-pyrazol-4-yl)-7- |
| | fluorobenzo[b]thiophene-3-carbonitrile |
| 25 | 2-amino-4-(3,5-dimethyl-1-(4-(1-(1-methylpyrrolidin-2-yl)ethoxy)-6-(pyridin-3- |
| | yl)pyrimidin-2-yl)-1H-pyrazol-4-yl)-7-fluorobenzo[b]thiophene-3-carbonitrile |
| 26 | 2-amino-7-fluoro-4-(1-(4-(6-(2-hydroxypropan-2-yl)pyridin-3-yl)-6-(1-(1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 27 | 5-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N,N-dimethylpicolinamide |
| 28 | 5-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N-methylpicolinamide |
| 29 | 2-amino-4-(3,5-dimethyl-1-(6-(1-(1-methylpyrrolidin-2-yl)ethoxy)-[4,5'-bipyrimidin]- |
| | 2-yl)-1H-pyrazol-4-yl)-7-fluorobenzo[b]thiophene-3-carbonitrile |
| 30 | 2-amino-4-(3,5-dimethyl-1-(4-(1-(1-methylpyrrolidin-2-yl)ethoxy)-6-(pyridin-4- |
| | yl)pyrimidin-2-yl)-1H-pyrazol-4-yl)-7-fluorobenzo[b]thiophene-3-carbonitrile |
| 31 | 1-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-1H-pyrazole-3- |
| | carboxamide |
| 32 | 1-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N-methyl-1H-pyrazole-3- |
| | carboxamide |
| 33 | 1-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N, N-dimethyl-1H- |
| | pyrazole-3-carboxamide |
| 34 | 1-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N-methyl-1H-pyrazole-4- |
| | carboxamide |
| 35 | 2-amino-4-(3,5-dimethyl-1-(4-(1-(1-methylpyrrolidin-2-yl)ethoxy)-6-(pyrimidin-5- |
| | yloxy)pyrimidin-2-yl)- 1H-pyrazol-4-yl)-7-fluorobenzo[b]thiophene-3-carbonitrile |
| 36 | 4-(1-(4-((1-acetylpiperidin-4-yl)oxy)-6-(1-(1-methylpyrrolidin-2-yl)ethoxy)pyrimidin- |
| | 2-yl)-3,5-dimethyl-1H-pyrazol-4-yl)-2-amino-7-fluorobenzo[b]thiophene-3- |
| | carbonitrile |
| 37 | 2-amino-4-(3,5-dimethyl-1-(6-(1-methyl-1H-pyrazol-4-yl)-2-(1-(1-methylpyrrolidin- |
| | 2-yl)ethoxy)pyrimidin-4-yl)-1H-pyrazol-4-yl)-7-fluorobenzo[b]thiophene-3- |
| | carbonitrile |
| 38 | 2-amino-4-(3,5-dimethyl-1-(2-(1-(1-methylpyrrolidin-2-yl)ethoxy)-6-(1-(oxetan-3-yl)- |
| | 1H-pyrazol-4-yl)pyrimidin-4-yl)-1H-pyrazol-4-yl)-7-fluorobenzo[b]thiophene-3- |
| | carbonitrile |
| 39 | 2-amino-4-(1-(6-(1-(difluoromethyl)-1H-pyrazol-4-yl)-2-(1-(1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 40 | 2-amino-4-(3,5-dimethyl-1-(2-(1-(1-methylpyrrolidin-2-yl)ethoxy)-6-(1 -(pyridin-3-yl)- |
| | 1H-pyrazol-4-yl)pyrimidin-4-yl)-1H-pyrazol-4-yl)-7-fluorobenzo[b]thiophene-3- |
| | carbonitrile |
| 41 | 2-amino-4-(1-(6-(1-benzyl-1H-pyrazol-4-yl)-2-(1-(1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 42 | 2-amino-4-(3,5-dimethyl-1-(2-(1-(1-methylpyrrolidin-2-yl)ethoxy)-6-(1-(pyridin-3- |
| | ylmethyl)-1H-pyrazol-4-yl)pyrimidin-4-yl)-1H-pyrazol-4-yl)-7- |
| | fluorobenzo[b]thiophene-3-carbonitrile |
| 43 | 2-amino-7-fluoro-4-(1-(6-(5-fluoro-1-methyl-1H-pyrazol-4-yl)-2-(1-(1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 44 | 2-amino-4-(1-(6-(1,3-dimethyl-1H-pyrazol-4-yl)-2-(1-(1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 45 | 4-(6-(4-(2-amino-3-cyano-7-fluorobenzo[b]thiophen-4-yl)-3,5-dimethyl-1H-pyrazol- |
| | 1-yl)-2-(1-(1-methylpyrrolidin-2-yl)ethoxy)pyrimidin-4-yl)-N-methylpicolinamide |
| 46 | 2-amino-4-(1-(6-(1-(cyanomethyl)-1H-pyrazol-4-yl)-2-(1-(1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 47 | 2-amino-4-(1-(6-(4-(cyclopropanecarbonyl)piperazin-1-yl)-2-(1-(1-methylpyrrolidin- |
| | 2-yl)ethoxy)pyrimidin-4-yl)-3,5-dimethyl-1H-pyrazol-4-yl)-7- |
| | fluorobenzo[b]thiophene-3-carbonitrile |
| 48 | 4-(1-(6-(4-acetylpiperazin-1-yl)-2-(1-(1-methylpyrrolidin-2-yl)ethoxy)pyrimidin-4-yl)- |
| | 3,5-dimethyl-1H-pyrazol-4-yl)-2-amino-7-fluorobenzo[b]thiophene-3-carbonitrile |
| 49 | 5-(6-(4-(2-amino-3-cyano-7-fluorobenzo[b]thiophen-4-yl)-3,5-dimethyl-1H-pyrazol- |
| | 1-yl)-2-(1-(1-methylpyrrolidin-2-yl)ethoxy)pyrimidin-4-yl)-N,N-dimethyl-5,6,7,8- |
| | tetrahydro-4H-pyrazolo[1,5-a][1,4]diazepine-2-carboxamide |
| 50 | 1-(6-(4-(2-amino-3-cyano-7-fluorobenzo[b]thiophen-4-yl)-3,5-dimethyl-1H-pyrazol- |
| | 1-yl)-2-(1-(1-methylpyrrolidin-2-yl)ethoxy)pyrimidin-4-yl)-1H-pyrazole-3- |
| | carboxamide |
| 51 | 4-(1-(6-(4-acetylpiperazin-1-yl)-5-fluoro-2-(1-(1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 52 | 4-(4-acetylpiperazin-1-yl)-6-(4-(2-amino-3-cyano-7-fluorobenzo[b]thiophen-4-yl)- |
| | 3,5-dimethyl-1H-pyrazol-1-yl)-2-(1-(1-methylpyrrolidin-2-yl)ethoxy)pyrimidine-5- |
| | carbonitrile |
| 53 | 2-amino-4-(1-(4-(4-(cyclopropanecarbonyl)piperazin-1-yl)-6-(1-(1-methylpyrrolidin- |
| | 2-yl)ethoxy)pyrimidin-2-yl)-3-methyl-1H-pyrazol-4-yl)-7-fluorobenzo[b]thiophene-3- |
| | carbonitrile |
| 54 | 2-amino-7-fluoro-4-(1-(2-((2-fluorotetrahydro-1H-pyrrolizin-7a(5H)-yl)methoxy)-6- |
| | (1-methyl-1H-pyrazol-4-yl)pyrimidin-4-yl)-3,5-dimethyl-1H-pyrazol-4- |
| | yl)benzo[b]thiophene-3-carbonitrile |
| 55 | 1-(2-(4-(2-amino-3-cyano-7-fluorobenzo[b]thiophen-4-yl)-3,5-dimethyl-1H-pyrazol- |
| | 1-yl)-6-methylpyrimidin-4-yl)-1H-pyrazole-3-carboxamide |
| 56 | 2-amino-4-(1-(2-(4-(cyclopropanecarbonyl)piperazin-1-yl)-6-(1-(1-methylpyrrolidin- |
| | 2-yl)ethoxy)pyrimidin-4-yl)-3,5-dimethyl-1H-pyrazol-4-yl)-7- |
| | fluorobenzo[b]thiophene-3-carbonitrile |
| 57 | 1-(2-(4-(2-amino-3-cyano-7-fluorobenzo[b]thiophen-4-yl)-3,5-dimethyl-1H-pyrazol- |
| | 1-yl)-6-(1-(1-methylpyrrolidin-2-yl)ethoxy)pyrimidin-4-yl)-N-(1-methylcyclopropyl)- |
| | 1H-pyrazole-3-carboxamide |
| 58 | 5-(6-(4-(2-amino-3-cyano-7-fluorobenzo[b]thiophen-4-yl)-3,5-dimethyl-1H-pyrazol- |
| | 1-yl)-2-(1-(1-methylpyrrolidin-2-yl)ethoxy)pyrimidin-4-yl)-N,N-dimethylpicolinamide |
| 59 | 2-amino-4-(3,5-dimethyl-1-(6-(1-methyl-6-oxo-1,6-dihydropyridin-3-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 60 | 4-(6-(4-(2-amino-3-cyano-7-fluorobenzo[b]thiophen-4-yl)-3,5-dimethyl-1H-pyrazol- |
| | 1-yl)-2-(1-(1-methylpyrrolidin-2-yl)ethoxy)pyrimidin-4-yl)-2-fluoro-N- |
| | methylbenzamide |
| 61 | 2-amino-4-(3,5-dimethyl-1-(6-(2-methyl-1-oxoisoindolin-5-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 62 | 2-amino-4-(3,5-dimethyl-1-(6-(3-methyl-[1,2,4]triazolo[4,3-a]pyridin-6-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 63 | methyl 2-(6-(4-(2-amino-3-cyano-7-fluorobenzo[b]thiophen-4-yl)-3,5-dimethyl-1H- |
| | pyrazol-1-yl)-2-(1-(1-methylpyrrolidin-2-yl)ethoxy)pyrimidin-4-yl)pyrrolidine-1- |
| | carboxylate |
| 64 | 2-amino-4-(3,5-dimethyl-1-(2-(1-(1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 65 | 2-amino-4-(3,5-dimethyl-1-(6-(6-methyl-5-oxo-2,6-diazaspiro[3.4]octan-2-yl)-2-(1- |
| | (1-methylpyrrolidin-2-yl)ethoxy)pyrimidin-4-yl)-1H-pyrazol-4-yl)-7- |
| | fluorobenzo[b]thiophene-3-carbonitrile |
| 66 | 4-(1-(6-(2-oxa-5-azabicyclo[2.2.1 ]heptan-5-yl)-2-(1-(1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 67 | 2-amino-4-(3,5-dimethyl-1-(2-(1-(1-methylpyrrolidin-2-yl)ethoxy)-6-(pyridin-4- |
| | yloxy)pyrimidin-4-yl)- 1H-pyrazol-4-yl)-7-fluorobenzo[b]thiophene-3-carbonitrile |
| 68 | 2-amino-7-fluoro-4-(1-(6-((3-fluoropyridin-4-yl)oxy)-2-(1-(1-methylpyrrolidin-2- |
| | yl)ethoxy)pyrimidin-4-yl)-3,5-dimethyl-1H-pyrazol-4-yl)benzo[b]thiophene-3- |
| | carbonitrile |
| 69 | 4-(1-(6-(1-acetylpiperidin-4-yl)-2-(1-(1-methylpyrrolidin-2-yl)ethoxy)pyrimidin-4-yl)- |
| | 3,5-dimethyl-1H-pyrazol-4-yl)-2-amino-7-fluorobenzo[b]thiophene-3-carbonitrile |
| 70 | 2-amino-4-(3,5-dimethyl-1-(4-(1-(methyl-d3)-1H-pyrazol-4-yl)-6-(1-(1- |
| | methylpyrrolidin-2-yl)ethoxy)pyridin-2-yl)-1H-pyrazol-4-yl)-7- |
| | fluorobenzo[b]thiophene-3-carbonitrile |
| 71 | 4-(6-(4-(2-amino-3-cyano-7-fluorobenzo[b]thiophen-4-yl)-3,5-dimethyl-1H-pyrazol- |
| | 1-yl)-2-((1-methylpyrrolidin-2-yl)methoxy)pyrimidin-4-yl)-N, N-dimethylpiperazine-1- |
| | carboxamide |
| 72 | 4-(6-(4-(2-amino-3-cyano-7-fluorobenzo[b]thiophen-4-yl)-3,5-dimethyl-1H-pyrazol- |
| | 1-yl)-2-((1-(dimethylamino)propan-2-yl)oxy)pyrimidin-4-yl)-N,N-dimethylpiperazine- |
| | 1 -carboxamide |
| 73 | 2-amino-4-(3,5-dimethyl-1-(6-(1-methyl-1H-pyrazol-4-yl)-2-(1-(1-methylpyrrolidin- |
| | 2-yl)ethoxy)pyrimidin-4-yl)-1H-pyrazol-4-yl)-7-fluorothieno[3,2-c]pyridine-3- |
| | carbonitrile |
| 74 | 2-amino-4-(3-chloro-5-methyl-1-(6-(1-methyl-1H-pyrazol-4-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 75 | 2-amino-7-fluoro-4-(3-(methoxymethyl)-5-methyl-1-(6-(1-methyl-1H-pyrazol-4-yl)- |
| | 2-(1-(1-methylpyrrolidin-2-yl)ethoxy)pyrimidin-4-yl)- 1H-pyrazol-4- |
| | yl)benzo[b]thiophene-3-carbonitrile |
| 76 | 2-amino-7-fluoro-4-(3-(methoxymethyl)-5-methyl-1-(6-(1-methyl-1H-pyrazol-4-yl)- |
| | 2-(1-(1-methylpyrrolidin-2-yl)ethoxy)pyrimidin-4-yl)- 1H-pyrazol-4- |
| | yl)benzo[b]thiophene-3-carbonitrile |
| 77 | 2-amino-4-(3-cyclopropyl-5-methyl-1-(6-(1-methyl-1H-pyrazol-4-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 78 | 2-amino-4-(3-(difluoromethyl)-5-methyl-1-(6-(1-methyl-1H-pyrazol-4-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 79 | 2-amino-4-(5-ethyl-3-methyl-1-(6-(1-methyl-1H-pyrazol-4-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 80 | 2-amino-7-fluoro-4-(3-methyl-1-(6-(1-methyl-1H-pyrazol-4-yl)-2-(1-(1- |
| | methylpyrrolidin-2-yl)ethoxy)pyrimidin-4-yl)-5-vinyl-1H-pyrazol-4- |
| | yl)benzo[b]thiophene-3-carbonitrile |
| 81 | 2-amino-7-fluoro-4-(3-(hydroxymethyl)-5-methyl-1-(6-(1-methyl-1H-pyrazol-4-yl)-2- |
| | (1-(1-methylpyrrolidin-2-yl)ethoxy)pyrimidin-4-yl)-1H-pyrazol-4- |
| | yl)benzo[b]thiophene-3-carbonitrile |
| 82 | 2-amino-7-fluoro-4-(5-(hydroxymethyl)-3-methyl-1-(6-(1-methyl-1H-pyrazol-4-yl)-2- |
| | (1-(1-methylpyrrolidin-2-yl)ethoxy)pyrimidin-4-yl)-1H-pyrazol-4- |
| | yl)benzo[b]thiophene-3-carbonitrile. |
| 83 | 4-(6-(4-(2-amino-3-cyano-7-fluorobenzo[b]thiophen-4-yl)-3,5-dimethyl-1H-pyrazol- |
| | 1-yl)-2-((1-((dimethylamino)methyl) cyclopropyl)methoxy)pyrimidin-4-yl)-N, N- |
| | dimethylpiperazine-1-carboxamide |
| 84 | 4-(6-(4-(2-amino-3-cyano-7-fluorobenzo[b]thiophen-4-yl)-3,5-dimethyl-1H-pyrazol- |
| | 1-yl)-2-(3-(dimethylamino)pyrrolidin-1-yl)pyrimidin-4-yl)-N,N-dimethylpiperazine-1- |
| | carboxamide |
| 85 | 4-(2-(3-acetamidopyrrolidin-1-yl)-6-(4-(2-amino-3-cyano-7-fluorobenzo[b]thiophen- |
| | 4-yl)-3,5-dimethyl-1H-pyrazol-1-yl)pyrimidin-4-yl)-N,N-dimethylpiperazine-1- |
| | carboxamide |
| 86 | 4-(6-(4-(2-amino-3-cyano-7-fluorobenzo[b]thiophen-4-yl)-3,5-dimethyl-1H-pyrazol- |
| | 1-yl)-2-(1-methylhexahydropyrrolo[3,4-b]pyrrol-5(1H)-yl)pyrimidin-4-yl)-2-fluoro-N- |
| | methylbenzamide |
| 87 | 4-(6-(4-(2-amino-3-cyano-7-fluorobenzo[b]thiophen-4-yl)-3,5-dimethyl-1H-pyrazol- |
| | 1-yl)-2-(1-methyloctahydro-6H-pyrrolo[3,4-b]pyridin-6-yl)pyrimidin-4-yl)-2-fluoro-N- |
| | methylbenzamide |
| 88 | 2-amino-4-(5-ethynyl-3-methyl-1-(6-(1-methyl-1H-pyrazol-4-yl)-2-(1-(1- |
| | methylpyrrolidin-2-yl)ethoxy)pyrimidin-4-yl)-1H-pyrazol-4-yl)-7- |
| | fluorobenzo[b]thiophene-3-carbonitrile |
| 89 | N-(1-(6-(4-(2-amino-3-cyano-7-fluorobenzo[b]thiophen-4-yl)-3,5-dimethyl-1H- |
| | pyrazol-1-yl)-2-(1-(1-methylpyrrolidin-2-yl)ethoxy)pyrimidin-4- |
| | yl)ethyl)cyclopropanecarboxamide |
| 90 | 2-amino-4-(1-(6-((1-(cyclopropanecarbonyl)azetidin-2-yl)methyl)-2-(1-(1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 91 | 6-(4-(2-amino-3-cyano-7-fluorobenzo[b]thiophen-4-yl)-3,5-dimethyl-1H-pyrazol-1- |
| | yl)-2-(1-(1-methylpyrrolidin-2-yl)ethoxy)-N-(1-(pyrimidin-5-yl)ethyl)pyrimidine-4- |
| | carboxamide |
| 92 | 2-amino-4-(5-ethyl-3-methyl-1-(6-(2-methyl-1-oxoisoindolin-5-yl)-2-(1-(1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 93 | 2-amino-4-(5-ethyl-3-methyl-1-(6-(2-methyl-1-oxoisoindolin-5-yl)-2-(1- |
| | methylhexahydropyrrolo[3,4-b]pyrrol-5(1H)-yl)pyrimidin-4-yl)-1H-pyrazol-4-yl)-7- |
| | fluorothieno[3,2-c]pyridine-3-carbonitrile |
| 94 | 5-(6-(4-(2-amino-3-cyano-7-fluorothieno[3,2-c]pyridin-4-yl)-5-ethyl-3-methyl-1H- |
| | pyrazol-1-yl)-2-(1-(1-methylpyrrolidin-2-yl)ethoxy)pyrimidin-4-yl)-N- |
| | methylpicolinamide |
| 95 | 4-(6-(4-(2-amino-3-cyano-7-fluorothieno[3,2-c]pyridin-4-yl)-5-ethyl-3-methyl-1H- |
| | pyrazol-1-yl)-2-(1-(1-methylpyrrolidin-2-yl)ethoxy)pyrimidin-4-yl)-2-fluoro-N- |
| | methylbenzamide |
| 96 | 4-(6-(4-(2-amino-3-cyano-7-fluorothieno[3,2-c]pyridin-4-yl)-5-ethyl-3-methyl-1H- |
| | pyrazol-1-yl)-2-(1-(1-methylpyrrolidin-2-yl)ethoxy)pyrimidin-4-yl)-N-cyclopropyl-2- |
| | fluorobenzamide |
| 97 | 5-(6-(4-(2-amino-3-cyano-7-fluorothieno[3,2-c]pyridin-4-yl)-5-ethyl-3-methyl-1H- |
| | pyrazol-1-yl)-2-(1-methylhexahydropyrrolo[3,4-b]pyrrol-5(1H)-yl)pyrimidin-4-yl)-3- |
| | fluoro-N-methylpicolinamide |
| 98 | 4-(6-(4-(2-amino-3-cyano-7-fluorothieno[3,2-c]pyridin-4-yl)-5-ethyl-3-methyl-1H- |
| | pyrazol-1-yl)-2-((3aS,6aS)-1-methylhexahydropyrrolo[3,4-b]pyrrol-5(1H)- |
| | yl)pyrimidin-4-yl)-N-cyclopropyl-2-fluorobenzamide |
| 99 | 5-(6-(4-(2-amino-3-cyano-7-fluorothieno[3,2-c]pyridin-4-yl)-5-ethyl-3-methyl-1H- |
| | pyrazol-1-yl)-2-(1-methylhexahydropyrrolo[3,4-b]pyrrol-5(1H)-yl)pyrimidin-4-yl)-N- |
| | cyclopropyl-3-fluoropicolinamide |
| 100 | 2-amino-4-(5-ethyl-1-(6-(2-methyl-1-oxoisoindolin-5-yl)-2-(1-(1-methylpyrrolidin-2- |
| | yl)ethoxy)pyrimidin-4-yl)-3-(trifluoromethyl)-1H-pyrazol-4-yl)-7-fluorothieno[3,2- |
| | c]pyridine-3-carbonitrile |
| 101 | 2-amino-4-(3,5-diethyl-1-(6-(2-methyl-1-oxoisoindolin-5-yl)-2-(1-(1- |
| | methylpyrrolidin-2-yl)ethoxy)pyrimidin-4-yl)-1H-pyrazol-4-yl)-7-fluorothieno[3,2- |
| | clpyridine-3-carbonitrile |
| 102 | 2-amino-4-(5-ethyl-3-methyl-1-(6-(2-methyl-1-oxoisoindolin-5-yl)-2-((1- |
| | methylpyrrolidin-2-yl)methoxy)pyrimidin-4-yl)-1H-pyrazol-4-yl)-7-fluorothieno[3,2- |
| | c]pyridine-3-carbonitrile |
| 103 | 2-amino-4-(1-(2-(3-(dimethylamino)pyrrolidin-1-yl)-6-(2-methyl-1-oxoisoindolin-5- |
| | yl)pyrimidin-4-yl)-5-ethyl-3-methyl-1H-pyrazol-4-yl)-7-fluorothieno[3,2-c]pyridine-3- |
| | carbonitrile |
| 104 | 2-amino-4-(5-ethyl-3-methyl-1-(6-(2-methyl-1-oxoisoindolin-5-yl)-2-(6-methyl-3,6- |
| | diazabicyclo[3.2.0]heptan-3-yl)pyrimidin-4-yl)-1H-pyrazol-4-yl)-7-fluorothieno[3,2- |
| | c]pyridine-3-carbonitrile |
| 105 | 2-amino-4-(5-ethyl-3-methyl-1-(6-(2-methyl-1-oxoisoindolin-5-yl)-2-(1- |
| | methyloctahydro-6H-pyrrolo[3,4-b]pyridin-6-yl)pyrimidin-4-yl)-1H-pyrazol-4-yl)-7- |
| | fluorothieno[3,2-c]pyridine-3-carbonitrile |
| 106 | 2-amino-4-(1-(2-(1-cyclopropylhexahydropyrrolo[3,4-b]pyrrol-5(1H)-yl)-6-(2- |
| | methyl-1-oxoisoindolin-5-yl)pyrimidin-4-yl)-5-ethyl-3-methyl-1H-pyrazol-4-yl)-7- |
| | fluorothieno[3,2-c]pyridine-3-carbonitrile |

In other embodiments, the compound of Formula (I) is selected from a compound in Table 2, and pharmaceutically acceptable salts thereof.

TABLE 2

| Entry | Compound Name |
|---|---|
| 1 | 4-(1-(4-(4-acetylpiperazin-1-yl)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 2 | 4-(1-(4-(4-acetylpiperazin-1-yl)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 3 | 4-(1-(4-(4-acetylpiperazin-1-yl)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 4 | 4-(1-(4-(4-acetylpiperazin-1-yl)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 5 | (Rₐ)-4-(1-(4-(4-acetylpiperazin-1-yl)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 6 | (Sₐ)-4-(1-(4-(4-acetylpiperazin-1-yl)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 7 | (Rₐ)-4-(1-(4-(4-acetylpiperazin-1-yl)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 8 | (Sₐ)-4-(1-(4-(4-acetylpiperazin-1-yl)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 9 | (Rₐ)-4-(1-(4-(4-acetylpiperazin-1-yl)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 10 | (Sₐ)-4-(1-(4-(4-acetylpiperazin-1-yl)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 11 | (Rₐ)-4-(1-(4-(4-acetylpiperazin-1-yl)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 12 | (Sₐ)-4-(1-(4-(4-acetylpiperazin-1-yl)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 13 | 1-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N- |
| | methylpyrrolidine-3-carboxamide |
| 14 | 1-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N- |
| | methylpyrrolidine-3-carboxamide |
| 15 | 1-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N- |
| | methylpyrrolidine-3-carboxamide |
| 16 | 1-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N- |
| | methylpyrrolidine-3-carboxamide |
| 17 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N- |
| | methylpyrrolidine-3-carboxamide |
| 18 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N- |
| | methylpyrrolidine-3-carboxamide |
| 19 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N- |
| | methylpyrrolidine-3-carboxamide |
| 20 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N- |
| | methylpyrrolidine-3-carboxamide |
| 21 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N- |
| | methylpyrrolidine-3-carboxamide |
| 22 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N- |
| | methylpyrrolidine-3-carboxamide |
| 23 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N- |
| | methylpyrrolidine-3-carboxamide |
| 24 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N- |
| | methylpyrrolidine-3-carboxamide |
| 25 | 2-amino-4-(3,5-dimethyl-1-(4-(4-methyl-3-oxopiperazin-1-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 26 | 2-amino-4-(3,5-dimethyl-1-(4-(4-methyl-3-oxopiperazin-1-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 27 | 2-amino-4-(3,5-dimethyl-1-(4-(4-methyl-3-oxopiperazin-1-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 28 | 2-amino-4-(3,5-dimethyl-1-(4-(4-methyl-3-oxopiperazin-1-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 29 | 2-amino-(Rₐ)-4-(3,5-dimethyl-1-(4-(4-methyl-3-oxopiperazin-1-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 30 | 2-amino-(Rₐ)-4-(3,5-dimethyl-1-(4-(4-methyl-3-oxopiperazin-1-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 31 | 2-amino-(Rₐ)-4-(3,5-dimethyl-1-(4-(4-methyl-3-oxopiperazin-1-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 32 | 2-amino-(Rₐ)-4-(3,5-dimethyl-1-(4-(4-methyl-3-oxopiperazin-1-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 33 | 2-amino-(Sₐ)-4-(3,5-dimethyl-1-(4-(4-methyl-3-oxopiperazin-1-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 34 | 2-amino-(Sₐ)-4-(3,5-dimethyl-1-(4-(4-methyl-3-oxopiperazin-1-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 35 | 2-amino-(Sₐ)-4-(3,5-dimethyl-1-(4-(4-methyl-3-oxopiperazin-1-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 36 | 2-amino-(Sₐ)-4-(3,5-dimethyl-1-(4-(4-methyl-3-oxopiperazin-1-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 37 | 2-amino-7-fluoro-4-(1-(4-(4-hydroxy-4-methylpiperidin-1-yl)-6-((S)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 38 | 2-amino-7-fluoro-4-(1-(4-(4-hydroxy-4-methylpiperidin-1-yl)-6-((S)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 39 | 2-amino-7-fluoro-4-(1-(4-(4-hydroxy-4-methylpiperidin-1-yl)-6-((R)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 40 | 2-amino-7-fluoro-4-(1-(4-(4-hydroxy-4-methylpiperidin-1-yl)-6-((R)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 41 | 2-amino-7-fluoro-(Rₐ)-4-(1-(4-(4-hydroxy-4-methylpiperidin-1-yl)-6-((S)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 42 | 2-amino-7-fluoro-(Rₐ)-4-(1-(4-(4-hydroxy-4-methylpiperidin-1-yl)-6-((S)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 43 | 2-amino-7-fluoro-(Rₐ)-4-(1-(4-(4-hydroxy-4-methylpiperidin-1-yl)-6-((R)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 44 | 2-amino-7-fluoro-(Rₐ)-4-(1-(4-(4-hydroxy-4-methylpiperidin-1-yl)-6-((R)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 45 | 2-amino-7-fluoro-(Rₐ)-4-(1-(4-(4-hydroxy-4-methylpiperidin-1-yl)-6-((S)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 46 | 2-amino-7-fluoro-(Sₐ)-4-(1-(4-(4-hydroxy-4-methylpiperidin-1-yl)-6-((S)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 47 | 2-amino-7-fluoro-(Sₐ)-4-(1-(4-(4-hydroxy-4-methylpiperidin-1-yl)-6-((R)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 48 | 2-amino-7-fluoro-(Sₐ)-4-(1-(4-(4-hydroxy-4-methylpiperidin-1-yl)-6-((R)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 49 | 4-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N,N- |
| | dimethylpiperazine-1-carboxamide |
| 50 | 4-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N,N- |
| | dimethylpiperazine-1-carboxamide |
| 51 | 4-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N,N- |
| | dimethylpiperazine-1-carboxamide |
| 52 | 4-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N,N- |
| | dimethylpiperazine-1-carboxamide |
| 53 | 4-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N,N- |
| | dimethylpiperazine-1-carboxamide |
| 54 | 4-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N,N- |
| | dimethylpiperazine-1-carboxamide |
| 55 | 4-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N,N- |
| | dimethylpiperazine-1-carboxamide |
| 56 | 4-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N,N- |
| | dimethylpiperazine-1-carboxamide |
| 57 | 4-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N,N- |
| | dimethylpiperazine-1-carboxamide |
| 58 | 4-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N,N- |
| | dimethylpiperazine-1-carboxamide |
| 59 | 4-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N,N- |
| | dimethylpiperazine-1-carboxamide |
| 60 | 4-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N,N- |
| | dimethylpiperazine-1-carboxamide |
| 61 | 4-(1-(4-(6-acetyl-2,6-diazaspiro[3.3]heptan-2-yl)-6-((S)-1-((S)-1-methylpyrrolidin- |
| | 2-yl)ethoxy)pyrimidin-2-yl)-3,5-dimethyl-1H-pyrazol-4-yl)-2-amino-7- |
| | fluorobenzo[b]thiophene-3-carbonitrile |
| 62 | 4-(1-(4-(6-acetyl-2,6-diazaspiro[3.3]heptan-2-yl)-6-((S)-1-((R)-1-methylpyrrolidin- |
| | 2-yl)ethoxy)pyrimidin-2-yl)-3,5-dimethyl-1H-pyrazol-4-yl)-2-amino-7- |
| | fluorobenzo[b]thiophene-3-carbonitrile |
| 63 | 4-(1-(4-(6-acetyl-2,6-diazaspiro[3.3]heptan-2-yl)-6-((R)-1-((S)-1-methylpyrrolidin- |
| | 2-yl)ethoxy)pyrimidin-2-yl)-3,5-dimethyl-1H-pyrazol-4-yl)-2-amino-7- |
| | fluorobenzo[b]thiophene-3-carbonitrile |
| 64 | 4-(1-(4-(6-acetyl-2,6-diazaspiro[3.3]heptan-2-yl)-6-((R)-1-((R)-1-methylpyrrolidin- |
| | 2-yl)ethoxy)pyrimidin-2-yl)-3,5-dimethyl-1H-pyrazol-4-yl)-2-amino-7- |
| | fluorobenzo[b]thiophene-3-carbonitrile |
| 65 | (Rₐ)-4-(1-(4-(6-acetyl-2,6-diazaspiro[3.3]heptan-2-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 66 | (Rₐ)-4-(1-(4-(6-acetyl-2,6-diazaspiro[3.3]heptan-2-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 67 | (Rₐ)-4-(1-(4-(6-acetyl-2,6-diazaspiro[3.3]heptan-2-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 68 | (Rₐ)-4-(1-(4-(6-acetyl-2,6-diazaspiro[3.3]heptan-2-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 69 | (Sₐ)-4-(1-(4-(6-acetyl-2,6-diazaspiro[3.3]heptan-2-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 70 | (Sₐ)-4-(1-(4-(6-acetyl-2,6-diazaspiro[3.3]heptan-2-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 71 | (Sₐ)-4-(1-(4-(6-acetyl-2,6-diazaspiro[3.3]heptan-2-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 72 | (Sₐ)-4-(1-(4-(6-acetyl-2,6-diazaspiro[3.3]heptan-2-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 73 | 2-amino-4-(1-(4-(4-(cyclopropanecarbonyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 74 | 2-amino-4-(1-(4-(4-(cyclopropanecarbonyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 75 | 2-amino-4-(1-(4-(4-(cyclopropanecarbonyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 76 | 2-amino-4-(1-(4-(4-(cyclopropanecarbonyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 77 | 2-amino-(Rₐ)-4-(1-(4-(4-(cyclopropanecarbonyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 78 | 2-amino-(Rₐ)-4-(1-(4-(4-(cyclopropanecarbonyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 79 | 2-amino-(Rₐ)-4-(1-(4-(4-(cyclopropanecarbonyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 80 | 2-amino-(Rₐ)-4-(1-(4-(4-(cyclopropanecarbonyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 81 | 2-amino-(Sₐ)-4-(1-(4-(4-(cyclopropanecarbonyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 82 | 2-amino-(Sₐ)-4-(1-(4-(4-(cyclopropanecarbonyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 83 | 2-amino-(Sₐ)-4-(1-(4-(4-(cyclopropanecarbonyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 84 | 2-amino-(Sₐ)-4-(1-(4-(4-(cyclopropanecarbonyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 85 | Methyl 4-(2-(4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4- |
| | yl)piperazine-1-carboxylate |
| 86 | methyl 4-(2-(4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4- |
| | yl)piperazine-1-carboxylate |
| 87 | methyl 4-(2-(4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4- |
| | yl)piperazine-1-carboxylate |
| 88 | methyl 4-(2-(4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4- |
| | yl)piperazine-1-carboxylate |
| 89 | methyl 4-(2-((Rₐ)-4-(2-amino-3-cyano-7-fluorobenzo[b]thiophen-4-yl)-3,5- |
| | dimethyl-1H-pyrazol-1-yl)-6-((S)-1-((S)-1-methylpyrrolidin-2-yl)ethoxy)pyrimidin-4- |
| | yl)piperazine-1-carboxylate |
| 90 | methyl 4-(2-((Rₐ)-4-(2-amino-3-cyano-7-fluorobenzo[b]thiophen-4-yl)-3,5- |
| | dimethyl-1H-pyrazol-1-yl)-6-((S)-1-((R)-1-methylpyrrolidin-2-yl)ethoxy)pyrimidin-4- |
| | yl)piperazine-1-carboxylate |
| 91 | methyl 4-(2-((Rₐ)-4-(2-amino-3-cyano-7-fluorobenzo[b]thiophen-4-yl)-3,5- |
| | dimethyl-1H-pyrazol-1-yl)-6-((R)-1-((S)-1-methylpyrrolidin-2-yl)ethoxy)pyrimidin-4- |
| | yl)piperazine-1-carboxylate |
| 92 | methyl 4-(2-((Rₐ)-4-(2-amino-3-cyano-7-fluorobenzo[b]thiophen-4-yl)-3,5- |
| | dimethyl-1H-pyrazol-1-yl)-6-((R)-1-((R)-1-methylpyrrolidin-2-yl)ethoxy)pyrimidin-4- |
| | yl)piperazine-1-carboxylate |
| 93 | methyl 4-(2-((Sₐ)-4-(2-amino-3-cyano-7-fluorobenzo[b]thiophen-4-yl)-3,5-dimethyl- |
| | 1H-pyrazol-1-yl)-6-((S)-1-((S)-1-methylpyrrolidin-2-yl)ethoxy)pyrimidin-4- |
| | yl)piperazine-1-carboxylate |
| 94 | methyl 4-(2-((Sₐ)-4-(2-amino-3-cyano-7-fluorobenzo[b]thiophen-4-yl)-3,5-dimethyl- |
| | 1H-pyrazol-1-yl)-6-((S)-1-((R)-1-methylpyrrolidin-2-yl)ethoxy)pyrimidin-4- |
| | yl)piperazine-1-carboxylate |
| 95 | methyl 4-(2-((Sₐ)-4-(2-amino-3-cyano-7-fluorobenzo[b]thiophen-4-yl)-3,5-dimethyl- |
| | 1H-pyrazol-1-yl)-6-((R)-1-((S)-1-methylpyrrolidin-2-yl)ethoxy)pyrimidin-4- |
| | yl)piperazine-1-carboxylate |
| 96 | methyl 4-(2-((Sₐ)-4-(2-amino-3-cyano-7-fluorobenzo[b]thiophen-4-yl)-3,5-dimethyl- |
| | 1H-pyrazol-1-yl)-6-((R)-1-((R)-1-methylpyrrolidin-2-yl)ethoxy)pyrimidin-4- |
| | yl)piperazine-1-carboxylate |
| 97 | 2-amino-4-(3,5-dimethyl-1-(4-(2-methyl-3-oxohexahydroimidazo[1,5-a]pyrazin- |
| | 7(1H)-yl)-6-((S)-1-((S)-1-methylpyrrolidin-2-yl)ethoxy)pyrimidin-2-yl)-1H-pyrazol-4- |
| | yl)-7-fluorobenzo[b]thiophene-3-carbonitrile |
| 98 | 2-amino-4-(3,5-dimethyl-1-(4-(2-methyl-3-oxohexahydroimidazo[1,5-a]pyrazin- |
| | 7(1H)-yl)-6-((S)-1-((R)-1-methylpyrrolidin-2-yl)ethoxy)pyrimidin-2-yl)-1H-pyrazol-4- |
| | yl)-7-fluorobenzo[b]thiophene-3-carbonitrile |
| 99 | 2-amino-4-(3,5-dimethyl-1-(4-(2-methyl-3-oxohexahydroimidazo[1,5-a]pyrazin- |
| | 7(1H)-yl)-6-((R)-1-((S)-1-methylpyrrolidin-2-yl)ethoxy)pyrimidin-2-yl)-1H-pyrazol-4- |
| | yl)-7-fluorobenzo[b]thiophene-3-carbonitrile |
| 100 | 2-amino-4-(3,5-dimethyl-1-(4-(2-methyl-3-oxohexahydroimidazo[1,5-a]pyrazin- |
| | 7(1H)-yl)-6-((R)-1-((R)-1-methylpyrrolidin-2-yl)ethoxy)pyrimidin-2-yl)-1H-pyrazol-4- |
| | yl)-7-fluorobenzo[b]thiophene-3-carbonitrile |
| 101 | 2-amino-(Rₐ)-4-(3,5-dimethyl-1-(4-(2-methyl-3-oxohexahydroimidazo[1,5- |
| | alpyrazin-7(1H)-yl)-6-((S)-1-((S)-1-methylpyrrolidin-2-yl)ethoxy)pyrimidin-2-yl)-1H- |
| | pyrazol-4-yl)-7-fluorobenzo[b]thiophene-3-carbonitrile |
| 102 | 2-amino-(Rₐ)-4-(3,5-dimethyl-1-(4-(2-methyl-3-oxohexahydroimidazo[1,5- |
| | alpyrazin-7(1H)-yl)-6-((S)-1-((R)-1-methylpyrrolidin-2-yl)ethoxy)pyrimidin-2-yl)-1H- |
| | pyrazol-4-yl)-7-fluorobenzo[b]thiophene-3-carbonitrile |
| 103 | 2-amino-(Rₐ)-4-(3,5-dimethyl-1-(4-(2-methyl-3-oxohexahydroimidazo[1,5- |
| | alpyrazin-7(1H)-yl)-6-((R)-1-((S)-1-methylpyrrolidin-2-yl)ethoxy)pyrimidin-2-yl)-1H- |
| | pyrazol-4-yl)-7-fluorobenzo[b]thiophene-3-carbonitrile |
| 104 | 2-amino-(Rₐ)-4-(3,5-dimethyl-1-(4-(2-methyl-3-oxohexahydroimidazo[1,5- |
| | alpyrazin-7(1H)-yl)-6-((R)-1-((R)-1-methylpyrrolidin-2-yl)ethoxy)pyrimidin-2-yl)-1H- |
| | pyrazol-4-yl)-7-fluorobenzo[b]thiophene-3-carbonitrile |
| 105 | 2-amino-(Sₐ)-4-(3,5-dimethyl-1-(4-(2-methyl-3-oxohexahydroimidazo[1,5- |
| | alpyrazin-7(1H)-yl)-6-((S)-1-((S)-1-methylpyrrolidin-2-yl)ethoxy)pyrimidin-2-yl)-1H- |
| | pyrazol-4-yl)-7-fluorobenzo[b]thiophene-3-carbonitrile |
| 106 | 2-amino-(Sₐ)-4-(3,5-dimethyl-1-(4-(2-methyl-3-oxohexahydroimidazo[1,5- |
| | alpyrazin-7(1H)-yl)-6-((S)-1-((R)-1-methylpyrrolidin-2-yl)ethoxy)pyrimidin-2-yl)-1H- |
| | pyrazol-4-yl)-7-fluorobenzo[b]thiophene-3-carbonitrile |
| 107 | 2-amino-(Sₐ)-4-(3,5-dimethyl-1-(4-(2-methyl-3-oxohexahydroimidazo[1,5- |
| | alpyrazin-7(1H)-yl)-6-((R)-1-((S)-1-methylpyrrolidin-2-yl)ethoxy)pyrimidin-2-yl)-1H- |
| | pyrazol-4-yl)-7-fluorobenzo[b]thiophene-3-carbonitrile |
| 108 | 2-amino-(Sₐ)-4-(3,5-dimethyl-1-(4-(2-methyl-3-oxohexahydroimidazo[1,5- |
| | alpyrazin-7(1H)-yl)-6-((R)-1-((R)-1-methylpyrrolidin-2-yl)ethoxy)pyrimidin-2-yl)-1H- |
| | pyrazol-4-yl)-7-fluorobenzo[b]thiophene-3-carbonitrile |
| 109 | 4-(1-(4-(6-acetyl-2,6-diazaspiro[3.4]octan-2-yl)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 110 | 4-(1-(4-(6-acetyl-2,6-diazaspiro[3.4]octan-2-yl)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 111 | 4-(1-(4-(6-acetyl-2,6-diazaspiro[3.4]octan-2-yl)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 112 | 4-(1-(4-(6-acetyl-2,6-diazaspiro[3.4]octan-2-yl)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 113 | (Rₐ)-4-(1-(4-(6-acetyl-2,6-diazaspiro[3.4]octan-2-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 114 | (Rₐ)-4-(1-(4-(6-acetyl-2,6-diazaspiro[3.4]octan-2-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 115 | (Rₐ)-4-(1-(4-(6-acetyl-2,6-diazaspiro[3.4]octan-2-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 116 | (Rₐ)-4-(1-(4-(6-acetyl-2,6-diazaspiro[3.4]octan-2-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 117 | (Sₐ)-4-(1-(4-(6-acetyl-2,6-diazaspiro[3.4]octan-2-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 118 | (Sₐ)-4-(1-(4-(6-acetyl-2,6-diazaspiro[3.4]octan-2-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 119 | (Sₐ)-4-(1-(4-(6-acetyl-2,6-diazaspiro[3.4]octan-2-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 120 | (Sₐ)-4-(1-(4-(6-acetyl-2,6-diazaspiro[3.4]octan-2-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 121 | 4-(1-(4-(3-acetyl-3,8-diazabicyclo[3.2.1]octan-8-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 122 | 4-(1-(4-((1R,5S)-3-acetyl-3,8-diazabicyclo[3.2.1]octan-8-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 123 | 4-(1-(4-((1R,5S)-3-acetyl-3,8-diazabicyclo[3.2.1]octan-8-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 124 | 4-(1-(4-((1S,5R)-3-acetyl-3,8-diazabicyclo[3.2.1]octan-8-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 125 | 4-(1-(4-((1S,5R)-3-acetyl-3,8-diazabicyclo[3.2.1]octan-8-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 126 | 4-(1-(4-((1S,5R)-3-acetyl-3,8-diazabicyclo[3.2.1]octan-8-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 127 | 4-(1-(4-((1S,5R)-3-acetyl-3,8-diazabicyclo[3.2.1]octan-8-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 128 | 4-(1-(4-((1S,5R)-3-acetyl-3,8-diazabicyclo[3.2.1]octan-8-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 129 | (Rₐ)-4-(1-(4-(3-acetyl-3,8-diazabicyclo[3.2.1]octan-8-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 130 | (Rₐ)-4-(1-(4-((1R,5S)-3-acetyl-3,8-diazabicyclo[3.2.1]octan-8-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 131 | (Rₐ)-4-(1-(4-((1R,5S)-3-acetyl-3,8-diazabicyclo[3.2.1]octan-8-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 132 | (Rₐ)-4-(1-(4-((1S,5R)-3-acetyl-3,8-diazabicyclo[3.2.1]octan-8-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 133 | (Rₐ)-4-(1-(4-((1S,5R)-3-acetyl-3,8-diazabicyclo[3.2.1]octan-8-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 134 | (Rₐ)-4-(1-(4-((1S,5R)-3-acetyl-3,8-diazabicyclo[3.2.1]octan-8-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 134 | (Rₐ)-4-(1-(4-((1S,5R)-3-acetyl-3,8-diazabicyclo[3.2.1]octan-8-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 135 | (Rₐ)-4-(1-(4-((1S,5R)-3-acetyl-3,8-diazabicyclo[3.2.1]octan-8-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 136 | (Sₐ)-4-(1-(4-(3-acetyl-3,8-diazabicyclo[3.2.1]octan-8-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 137 | (Sₐ)-4-(1-(4-((1R,5S)-3-acetyl-3,8-diazabicyclo[3.2.1]octan-8-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 138 | (Sₐ)-4-(1-(4-((1R,5S)-3-acetyl-3,8-diazabicyclo[3.2.1]octan-8-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 139 | (Sₐ)-4-(1-(4-((1S,5R)-3-acetyl-3,8-diazabicyclo[3.2.1]octan-8-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 140 | (Sₐ)-4-(1-(4-((1S,5R)-3-acetyl-3,8-diazabicyclo[3.2.1]octan-8-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 141 | (Sₐ)-4-(1-(4-((1S,5R)-3-acetyl-3,8-diazabicyclo[3.2.1]octan-8-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 142 | (Sₐ)-4-(1-(4-((1S,5R)-3-acetyl-3,8-diazabicyclo[3.2.1]octan-8-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 143 | (Sₐ)-4-(1-(4-((1S,5R)-3-acetyl-3,8-diazabicyclo[3.2.1]octan-8-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 144 | 4-(1-(4-((S)-4-acetyl-3-(cyanomethyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 145 | 4-(1-(4-((S)-4-acetyl-3-(cyanomethyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 146 | 4-(1-(4-((S)-4-acetyl-3-(cyanomethyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 147 | 4-(1-(4-((R)-4-acetyl-3-(cyanomethyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 148 | 4-(1-(4-((R)-4-acetyl-3-(cyanomethyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 149 | 4-(1-(4-((R)-4-acetyl-3-(cyanomethyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 150 | 4-(1-(4-((R)-4-acetyl-3-(cyanomethyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 151 | 4-(1-(4-((S)-4-acetyl-3-(cyanomethyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 152 | (Rₐ)-4-(1-(4-((S)-4-acetyl-3-(cyanomethyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 153 | (Rₐ)-4-(1-(4-((S)-4-acetyl-3-(cyanomethyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 154 | (Rₐ)-4-(1-(4-((S)-4-acetyl-3-(cyanomethyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 155 | (Rₐ)-4-(1-(4-((R)-4-acetyl-3-(cyanomethyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 156 | (Rₐ)-4-(1-(4-((R)-4-acetyl-3-(cyanomethyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 157 | (Rₐ)-4-(1-(4-((R)-4-acetyl-3-(cyanomethyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 158 | (Rₐ)-4-(1-(4-((R)-4-acetyl-3-(cyanomethyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 159 | (Rₐ)-4-(1-(4-((S)-4-acetyl-3-(cyanomethyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 160 | (Sₐ)-4-(1-(4-((S)-4-acetyl-3-(cyanomethyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 161 | (Sₐ)-4-(1-(4-((S)-4-acetyl-3-(cyanomethyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 162 | (Sₐ)-4-(1-(4-((S)-4-acetyl-3-(cyanomethyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 163 | (Sₐ)-4-(1-(4-((R)-4-acetyl-3-(cyanomethyl)piperazin-1-yl)-6-((S)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 164 | (Sₐ)-4-(1-(4-((R)-4-acetyl-3-(cyanomethyl)piperazin-1-yl)-6-((R)-1-((S)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 165 | (Sₐ)-4-(1-(4-((R)-4-acetyl-3-(cyanomethyl)piperazin-1-yl)-6-((S)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 166 | (Sₐ)-4-(1-(4-((R)-4-acetyl-3-(cyanomethyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 167 | (Sₐ)-4-(1-(4-((S)-4-acetyl-3-(cyanomethyl)piperazin-1-yl)-6-((R)-1-((R)-1- |
| | methylpyrrolidin-2-yl)ethoxy)pyrimidin-2-yl)-3,5-dimethyl-1H-pyrazol-4-yl)-2- |
| | amino-7-fluorobenzo[b]thiophene-3-carbonitrile |
| 168 | 2-amino-4-(1-(4-(5,6-dihydroimidazo[1,5-a]pyrazin-7(8H)-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 169 | 2-amino-4-(1-(4-(5,6-dihydroimidazo[1,5-a]pyrazin-7(8H)-yl)-6-((S)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 170 | 2-amino-4-(1-(4-(5,6-dihydroimidazo[1,5-a]pyrazin-7(8H)-yl)-6-((R)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 171 | 2-amino-4-(1-(4-(5,6-dihydroimidazo[1,5-a]pyrazin-7(8H)-yl)-6-((R)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 172 | 2-amino-(Rₐ)-4-(1-(4-(5,6-dihydroimidazo[1,5-a]pyrazin-7(8H)-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 173 | 2-amino-(Rₐ)-4-(1-(4-(5,6-dihydroimidazo[1,5-a]pyrazin-7(8H)-yl)-6-((S)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 174 | 2-amino-(Rₐ)-4-(1-(4-(5,6-dihydroimidazo[1,5-a]pyrazin-7(8H)-yl)-6-((R)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 175 | 2-amino-(Rₐ)-4-(1-(4-(5,6-dihydroimidazo[1,5-a]pyrazin-7(8H)-yl)-6-((R)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 176 | 2-amino-(Sₐ)-4-(1-(4-(5,6-dihydroimidazo[1,5-a]pyrazin-7(8H)-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 177 | 2-amino-(Sₐ)-4-(1-(4-(5,6-dihydroimidazo[1,5-a]pyrazin-7(8H)-yl)-6-((S)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 178 | 2-amino-(Sₐ)-4-(1-(4-(5,6-dihydroimidazo[1,5-a]pyrazin-7(8H)-yl)-6-((R)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 179 | 2-amino-(Sₐ)-4-(1-(4-(5,6-dihydroimidazo[1,5-a]pyrazin-7(8H)-yl)-6-((R)-1-((R)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 180 | 2-amino-4-(1-(4-(5,6-dihydro-[1,2,4]triazolo[4,3-a]pyrazin-7(8H)-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 181 | 2-amino-4-(1-(4-(5,6-dihydro-[1,2,4]triazolo[4,3-a]pyrazin-7(8H)-yl)-6-((S)-1-((R)- |
| | 1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 182 | 2-amino-4-(1-(4-(5,6-dihydro-[1,2,4]triazolo[4,3-a]pyrazin-7(8H)-yl)-6-((R)-1-((S)- |
| | 1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 183 | 2-amino-4-(1-(4-(5,6-dihydro-[1,2,4]triazolo[4,3-a]pyrazin-7(8H)-yl)-6-((R)-1-((R)- |
| | 1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 184 | 2-amino-(Rₐ)-4-(1-(4-(5,6-dihydro-[1,2,4]triazolo[4,3-a]pyrazin-7(8H)-yl)-6-((S)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 185 | 2-amino-(Rₐ)-4-(1-(4-(5,6-dihydro-[1,2,4]triazolo[4,3-a]pyrazin-7(8H)-yl)-6-((S)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 186 | 2-amino-(Rₐ)-4-(1-(4-(5,6-dihydro-[1,2,4]triazolo[4,3-a]pyrazin-7(8H)-yl)-6-((R)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 187 | 2-amino-(Rₐ)-4-(1-(4-(5,6-dihydro-[1,2,4]triazolo[4,3-a]pyrazin-7(8H)-yl)-6-((R)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 188 | 2-amino-(Sₐ)-4-(1-(4-(5,6-dihydro-[1,2,4]triazolo[4,3-a]pyrazin-7(8H)-yl)-6-((S)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 189 | 2-amino-(Sₐ)-4-(1-(4-(5,6-dihydro-[1,2,4]triazolo[4,3-a]pyrazin-7(8H)-yl)-6-((S)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 190 | 2-amino-(Sₐ)-4-(1-(4-(5,6-dihydro-[1,2,4]triazolo[4,3-a]pyrazin-7(8H)-yl)-6-((R)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 191 | 2-amino-(Sₐ)-4-(1-(4-(5,6-dihydro-[1,2,4]triazolo[4,3-a]pyrazin-7(8H)-yl)-6-((R)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 192 | 2-amino-4-(3,5-dimethyl-1-(4-((S)-1-((S)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 193 | 2-amino-4-(3,5-dimethyl-1-(4-((S)-1-((R)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 194 | 2-amino-4-(3,5-dimethyl-1-(4-((R)-1-((S)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 195 | 2-amino-4-(3,5-dimethyl-1-(4-((R)-1-((R)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 196 | 2-amino-(Rₐ)-4-(3,5-dimethyl-1-(4-((S)-1-((S)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 197 | 2-amino-(Rₐ)-4-(3,5-dimethyl-1-(4-((S)-1-((R)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 198 | 2-amino-(Rₐ)-4-(3,5-dimethyl-1-(4-((R)-1-((S)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 199 | 2-amino-(Rₐ)-4-(3,5-dimethyl-1-(4-((R)-1-((R)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 200 | 2-amino-(Sₐ)-4-(3,5-dimethyl-1-(4-((S)-1-((S)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 201 | 2-amino-(Sₐ)-4-(3,5-dimethyl-1-(4-((S)-1-((R)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 202 | 2-amino-(Sₐ)-4-(3,5-dimethyl-1-(4-((R)-1-((S)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 203 | 2-amino-(Sₐ)-4-(3,5-dimethyl-1-(4-((R)-1-((R)-1-methylpyrrolidin-2-yl)ethoxy)-6-(4- |
| | (methylsulfonyl)piperazin-1-yl)pyrimidin-2-yl)-1H-pyrazol-4-yl)-7- |
| | fluorobenzo[b]thiophene-3-carbonitrile |
| 204 | 2-amino-4-(3,5-dimethyl-1-(4-((S)-1-((S)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 205 | 2-amino-4-(3,5-dimethyl-1-(4-((S)-1-((R)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 206 | 2-amino-4-(3,5-dimethyl-1-(4-((R)-1-((S)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 207 | 2-amino-4-(3,5-dimethyl-1-(4-((R)-1-((R)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 208 | 2-amino-(Rₐ)-4-(3,5-dimethyl-1-(4-((S)-1-((S)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 209 | 2-amino-(Rₐ)-4-(3,5-dimethyl-1-(4-((S)-1-((R)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 210 | 2-amino-(Rₐ)-4-(3,5-dimethyl-1-(4-((R)-1-((S)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 211 | 2-amino-(Rₐ)-4-(3,5-dimethyl-1-(4-((R)-1-((R)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 212 | 2-amino-(Sₐ)-4-(3,5-dimethyl-1-(4-((S)-1-((S)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 213 | 2-amino-(Sₐ)-4-(3,5-dimethyl-1-(4-((S)-1-((R)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 214 | 2-amino-(Sₐ)-4-(3,5-dimethyl-1-(4-((R)-1-((S)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 215 | 2-amino-(Sₐ)-4-(3,5-dimethyl-1-(4-((R)-1-((R)-1-methylpyrrolidin-2-yl)ethoxy)-6-(6- |
| | oxohexahydropyrrolo[1,2-a]pyrazin-2(1H)-yl)pyrimidin-2-yl)-1H-pyrazol-4-yl)-7- |
| | fluorobenzo[b]thiophene-3-carbonitrile |
| 216 | 2-amino-4-(1-(4-(5,6-dihydro-[1,2,4]triazolo[1,5-a]pyrazin-7(8H)-yl)-6-((S)-1-((S)-1- |
| | methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 217 | 2-amino-4-(1-(4-(5,6-dihydro-[1,2,4]triazolo[1,5-a]pyrazin-7(8H)-yl)-6-((R)-1-((S)- |
| | 1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 218 | 2-amino-4-(1-(4-(5,6-dihydro-[1,2,4]triazolo[1,5-a]pyrazin-7(8H)-yl)-6-((S)-1-((R)- |
| | 1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 219 | 2-amino-4-(1-(4-(5,6-dihydro-[1,2,4]triazolo[1,5-a]pyrazin-7(8H)-yl)-6-((R)-1-((R)- |
| | 1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 220 | 2-amino-(Rₐ)-4-(1-(4-(5,6-dihydro-[1,2,4]triazolo[1,5-a]pyrazin-7(8H)-yl)-6-((S)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 221 | 2-amino-(Rₐ)-4-(1-(4-(5,6-dihydro-[1,2,4]triazolo[1,5-a]pyrazin-7(8H)-yl)-6-((R)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 222 | 2-amino-(Rₐ)-4-(1-(4-(5,6-dihydro-[1,2,4]triazolo[1,5-a]pyrazin-7(8H)-yl)-6-((S)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 223 | 2-amino-(Rₐ)-4-(1-(4-(5,6-dihydro-[1,2,4]triazolo[1,5-a]pyrazin-7(8H)-yl)-6-((R)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 224 | 2-amino-(Sₐ)-4-(1-(4-(5,6-dihydro-[1,2,4]triazolo[1,5-a]pyrazin-7(8H)-yl)-6-((S)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 225 | 2-amino-(Sₐ)-4-(1-(4-(5,6-dihydro-[1,2,4]triazolo[1,5-a]pyrazin-7(8H)-yl)-6-((R)-1- |
| | ((S)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 226 | 2-amino-(Sₐ)-4-(1-(4-(5,6-dihydro-[1,2,4]triazolo[1,5-a]pyrazin-7(8H)-yl)-6-((S)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 227 | 2-amino-(Sₐ)-4-(1-(4-(5,6-dihydro-[1,2,4]triazolo[1,5-a]pyrazin-7(8H)-yl)-6-((R)-1- |
| | ((R)-1-methylpyrrolidin-2-yl)ethoxy)pyrimidin-2-yl)-3,5-dimethyl-1H-pyrazol-4-yl)-7- |
| | fluorobenzo[b]thiophene-3-carbonitrile |
| 228 | 2-amino-4-(3,5-dimethyl-1-(4-((S)-1-((S)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 229 | 2-amino-4-(3,5-dimethyl-1-(4-((S)-1-((R)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 230 | 2-amino-4-(3,5-dimethyl-1-(4-((R)-1-((S)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 231 | 2-amino-4-(3,5-dimethyl-1-(4-((R)-1-((R)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 232 | 2-amino-(Rₐ)-4-(3,5-dimethyl-1-(4-((S)-1-((S)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 233 | 2-amino-(Rₐ)-4-(3,5-dimethyl-1-(4-((S)-1-((R)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 234 | 2-amino-(Rₐ)-4-(3,5-dimethyl-1-(4-((R)-1-((S)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 235 | 2-amino-(Rₐ)-4-(3,5-dimethyl-1-(4-((R)-1-((R)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 236 | 2-amino-(Sₐ)-4-(3,5-dimethyl-1-(4-((S)-1-((S)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 237 | 2-amino-(Sₐ)-4-(3,5-dimethyl-1-(4-((S)-1-((R)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 238 | 2-amino-(Sₐ)-4-(3,5-dimethyl-1-(4-((R)-1-((S)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 239 | 2-amino-(Sₐ)-4-(3,5-dimethyl-1-(4-((R)-1-((R)-1-methylpyrrolidin-2-yl)ethoxy)-6- |
| | ((pyridin-3-ylmethyl)amino)pyrimidin-2-yl)-1H-pyrazol-4-yl)-7- |
| | fluorobenzo[b]thiophene-3-carbonitrile |
| 240 | 2-amino-4-(3,5-dimethyl-1-(4-((S)-1-((S)-1-methylpyrrolidin-2-yl)ethoxy)-6-((1- |
| | (pyridin-3-yl)ethyl)amino)pyrimidin-2-yl)-1H-pyrazol-4-yl)-7- |
| | fluorobenzo[b]thiophene-3-carbonitrile |
| 241 | 2-amino-4-(3,5-dimethyl-1-(4-((S)-1-((R)-1-methylpyrrolidin-2-yl)ethoxy)-6-(((S)-1- |
| | (pyridin-3-yl)ethyl)amino)pyrimidin-2-yl)-1H-pyrazol-4-yl)-7- |
| | fluorobenzo[b]thiophene-3-carbonitrile |
| 242 | 2-amino-4-(3,5-dimethyl-1-(4-((S)-1-((S)-1-methylpyrrolidin-2-yl)ethoxy)-6-(((R)-1- |
| | (pyridin-3-yl)ethyl)amino)pyrimidin-2-yl)-1H-pyrazol-4-yl)-7- |
| | fluorobenzo[b]thiophene-3-carbonitrile |
| 243 | 2-amino-4-(3,5-dimethyl-1-(4-((R)-1-((S)-1-methylpyrrolidin-2-yl)ethoxy)-6-(((S)-1- |
| | (pyridin-3-yl)ethyl)amino)pyrimidin-2-yl)-1H-pyrazol-4-yl)-7- |
| | fluorobenzo[b]thiophene-3-carbonitrile |
| 244 | 2-amino-4-(3,5-dimethyl-1-(4-((R)-1-((R)-1-methylpyrrolidin-2-yl)ethoxy)-6-(((S)-1- |
| | (pyridin-3-yl)ethyl)amino)pyrimidin-2-yl)-1H-pyrazol-4-yl)-7- |
| | fluorobenzo[b]thiophene-3-carbonitrile |
| 245 | 2-amino-4-(3,5-dimethyl-1-(4-((R)-1-((S)-1-methylpyrrolidin-2-yl)ethoxy)-6-(((R)-1- |
| | (pyridin-3-yl)ethyl)amino)pyrimidin-2-yl)-1H-pyrazol-4-yl)-7- |
| | fluorobenzo[b]thiophene-3-carbonitrile |
| 246 | 2-amino-4-(3,5-dimethyl-1-(4-((R)-1-((R)-1-methylpyrrolidin-2-yl)ethoxy)-6-(((R)-1- |
| | (pyridin-3-yl)ethyl)amino)pyrimidin-2-yl)-1H-pyrazol-4-yl)-7- |
| | fluorobenzo[b]thiophene-3-carbonitrile |
| 247 | 2-amino-4-(3,5-dimethyl-1-(4-((S)-1-((R)-1-methylpyrrolidin-2-yl)ethoxy)-6-(((R)-1- |
| | (pyridin-3-yl)ethyl)amino)pyrimidin-2-yl)-1H-pyrazol-4-yl)-7- |
| | fluorobenzo[b]thiophene-3-carbonitrile |
| 248 | 2-amino-(Rₐ)-4-(3,5-dimethyl-1-(4-((S)-1-((S)-1-methylpyrrolidin-2-yl)ethoxy)-6- |
| | ((1-(pyridin-3-yl)ethyl)amino)pyrimidin-2-yl)-1H-pyrazol-4-yl)-7- |
| | fluorobenzo[b]thiophene-3-carbonitrile |
| 249 | 2-amino-(Rₐ)-4-(3,5-dimethyl-1-(4-((S)-1-((R)-1-methylpyrrolidin-2-yl)ethoxy)-6- |
| | (((S)-1-(pyridin-3-yl)ethyl)amino)pyrimidin-2-yl)-1H-pyrazol-4-yl)-7- |
| | fluorobenzo[b]thiophene-3-carbonitrile |
| 250 | 2-amino-(Rₐ)-4-(3,5-dimethyl-1-(4-((S)-1-((S)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 251 | 2-amino-(Rₐ)-4-(3,5-dimethyl-1-(4-((R)-1-((S)-1-methylpyrrolidin-2-yl)ethoxy)-6- |
| | (((S)-1-(pyridin-3-yl)ethyl)amino)pyrimidin-2-yl)-1H-pyrazol-4-yl)-7- |
| | fluorobenzo[b]thiophene-3-carbonitrile |
| 252 | 2-amino-(Rₐ)-4-(3,5-dimethyl-1-(4-((R)-1-((R)-1-methylpyrrolidin-2-yl)ethoxy)-6- |
| | (((S)-1-(pyridin-3-yl)ethyl)amino)pyrimidin-2-yl)-1H-pyrazol-4-yl)-7- |
| | fluorobenzo[b]thiophene-3-carbonitrile |
| 253 | 2-amino-(Rₐ)-4-(3,5-dimethyl-1-(4-((R)-1-((S)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 254 | 2-amino-(Rₐ)-4-(3,5-dimethyl-1-(4-((R)-1-((R)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 255 | 2-amino-(Rₐ)-4-(3,5-dimethyl-1-(4-((S)-1-((R)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 256 | 2-amino-(Sₐ)-4-(3,5-dimethyl-1-(4-((S)-1-((S)-1-methylpyrrolidin-2-yl)ethoxy)-6-((1- |
| | (pyridin-3-yl)ethyl)amino)pyrimidin-2-yl)-1H-pyrazol-4-yl)-7- |
| | fluorobenzo[b]thiophene-3-carbonitrile |
| 257 | 2-amino-(Sₐ)-4-(3,5-dimethyl-1-(4-((S)-1-((R)-1-methylpyrrolidin-2-yl)ethoxy)-6- |
| | (((S)-1-(pyridin-3-yl)ethyl)amino)pyrimidin-2-yl)-1H-pyrazol-4-yl)-7- |
| | fluorobenzo[b]thiophene-3-carbonitrile |
| 258 | 2-amino-(Sₐ)-4-(3,5-dimethyl-1-(4-((S)-1-((S)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 259 | 2-amino-(Sₐ)-4-(3,5-dimethyl-1-(4-((R)-1-((S)-1-methylpyrrolidin-2-yl)ethoxy)-6- |
| | (((S)-1-(pyridin-3-yl)ethyl)amino)pyrimidin-2-yl)-1H-pyrazol-4-yl)-7- |
| | fluorobenzo[b]thiophene-3-carbonitrile |
| 260 | 2-amino-(Sₐ)-4-(3,5-dimethyl-1-(4-((R)-1-((R)-1-methylpyrrolidin-2-yl)ethoxy)-6- |
| | (((S)-1-(pyridin-3-yl)ethyl)amino)pyrimidin-2-yl)-1H-pyrazol-4-yl)-7- |
| | fluorobenzo[b]thiophene-3-carbonitrile |
| 261 | 2-amino-(Sₐ)-4-(3,5-dimethyl-1-(4-((R)-1-((S)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 262 | 2-amino-(Sₐ)-4-(3,5-dimethyl-1-(4-((R)-1-((R)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 263 | 2-amino-(Sₐ)-4-(3,5-dimethyl-1-(4-((S)-1-((R)-1-methylpyrrolidin-2-yl)ethoxy)-6- |
| | (((R)-1-(pyridin-3-yl)ethyl)amino)pyrimidin-2-yl)-1H-pyrazol-4-yl)-7- |
| | fluorobenzo[b]thiophene-3-carbonitrile |
| 264 | 4-(1-(4-((1-acetylpiperidin-4-yl)amino)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 265 | 4-(1-(4-((1-acetylpiperidin-4-yl)amino)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 266 | 4-(1-(4-((1-acetylpiperidin-4-yl)amino)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 267 | 4-(1-(4-((1-acetylpiperidin-4-yl)amino)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 268 | (Rₐ)-4-(1-(4-((1-acetylpiperidin-4-yl)amino)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 269 | (Rₐ)-4-(1-(4-((1-acetylpiperidin-4-yl)amino)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 270 | (Rₐ)-4-(1-(4-((1-acetylpiperidin-4-yl)amino)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 271 | (Rₐ)-4-(1-(4-((1-acetylpiperidin-4-yl)amino)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 272 | (Sₐ)-4-(1-(4-((1-acetylpiperidin-4-yl)amino)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 273 | (Sₐ)-4-(1-(4-((1-acetylpiperidin-4-yl)amino)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 274 | (Sₐ)-4-(1-(4-((1-acetylpiperidin-4-yl)amino)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 275 | (Sₐ)-4-(1-(4-((1-acetylpiperidin-4-yl)amino)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 276 | 2-amino-4-(3,5-dimethyl-1-(4-(1-methyl-1H-pyrazol-4-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 277 | 2-amino-4-(3,5-dimethyl-1-(4-(1-methyl-1H-pyrazol-4-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 278 | 2-amino-4-(3,5-dimethyl-1-(4-(1-methyl-1H-pyrazol-4-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 279 | 2-amino-4-(3,5-dimethyl-1-(4-(1-methyl-1H-pyrazol-4-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 280 | 2-amino-(Rₐ)-4-(3,5-dimethyl-1-(4-(1-methyl-1H-pyrazol-4-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 281 | 2-amino-(Rₐ)-4-(3,5-dimethyl-1-(4-(1-methyl-1H-pyrazol-4-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 282 | 2-amino-(Rₐ)-4-(3,5-dimethyl-1-(4-(1-methyl-1H-pyrazol-4-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 283 | 2-amino-(Rₐ)-4-(3,5-dimethyl-1-(4-(1-methyl-1H-pyrazol-4-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 284 | 2-amino-(Sₐ)-4-(3,5-dimethyl-1-(4-(1-methyl-1H-pyrazol-4-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 285 | 2-amino-(Sₐ)-4-(3,5-dimethyl-1-(4-(1-methyl-1H-pyrazol-4-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 286 | 2-amino-(Sₐ)-4-(3,5-dimethyl-1-(4-(1-methyl-1H-pyrazol-4-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 287 | 2-amino-(Sₐ)-4-(3,5-dimethyl-1-(4-(1-methyl-1H-pyrazol-4-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 288 | 2-amino-4-(3,5-dimethyl-1-(4-(1-methyl-1H-pyrazol-3-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 289 | 2-amino-4-(3,5-dimethyl-1-(4-(1-methyl-1H-pyrazol-3-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 290 | 2-amino-4-(3,5-dimethyl-1-(4-(1-methyl-1H-pyrazol-3-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 291 | 2-amino-4-(3,5-dimethyl-1-(4-(1-methyl-1H-pyrazol-3-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 292 | 2-amino-(Rₐ)-4-(3,5-dimethyl-1-(4-(1-methyl-1H-pyrazol-3-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 293 | 2-amino-(Rₐ)-4-(3,5-dimethyl-1-(4-(1-methyl-1H-pyrazol-3-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 294 | 2-amino-(Rₐ)-4-(3,5-dimethyl-1-(4-(1-methyl-1H-pyrazol-3-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 295 | 2-amino-(Rₐ)-4-(3,5-dimethyl-1-(4-(1-methyl-1H-pyrazol-3-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 296 | 2-amino-(Sₐ)-4-(3,5-dimethyl-1-(4-(1-methyl-1H-pyrazol-3-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 297 | 2-amino-(Sₐ)-4-(3,5-dimethyl-1-(4-(1-methyl-1H-pyrazol-3-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 298 | 2-amino-(Sₐ)-4-(3,5-dimethyl-1-(4-(1-methyl-1H-pyrazol-3-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 299 | 2-amino-(Sₐ)-4-(3,5-dimethyl-1-(4-(1-methyl-1H-pyrazol-3-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 300 | 2-amino-4-(3,5-dimethyl-1-(4-(3-methylisoxazol-5-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 301 | 2-amino-4-(3,5-dimethyl-1-(4-(3-methylisoxazol-5-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 302 | 2-amino-4-(3,5-dimethyl-1-(4-(3-methylisoxazol-5-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 303 | 2-amino-4-(3,5-dimethyl-1-(4-(3-methylisoxazol-5-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 304 | 2-amino-(Rₐ)-4-(3,5-dimethyl-1-(4-(3-methylisoxazol-5-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 305 | 2-amino-(Rₐ)-4-(3,5-dimethyl-1-(4-(3-methylisoxazol-5-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 306 | 2-amino-(Rₐ)-4-(3,5-dimethyl-1-(4-(3-methylisoxazol-5-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 307 | 2-amino-(Rₐ)-4-(3,5-dimethyl-1-(4-(3-methylisoxazol-5-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 308 | 2-amino-(Sₐ)-4-(3,5-dimethyl-1-(4-(3-methylisoxazol-5-yl)-6-((S)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 309 | 2-amino-(Sₐ)-4-(3,5-dimethyl-1-(4-(3-methylisoxazol-5-yl)-6-((S)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 310 | 2-amino-(Sₐ)-4-(3,5-dimethyl-1-(4-(3-methylisoxazol-5-yl)-6-((R)-1-((S)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 311 | 2-amino-(Sₐ)-4-(3,5-dimethyl-1-(4-(3-methylisoxazol-5-yl)-6-((R)-1-((R)-1- |
| | methylpyrrolidin-2-yl)ethoxy)pyrimidin-2-yl)-1H-pyrazol-4-yl)-7- |
| | fluorobenzo[b]thiophene-3-carbonitrile |
| 312 | 2-amino-4-(3,5-dimethyl-1-(4-(1-methyl-6-oxo-1,6-dihydropyridin-3-yl)-6-((S)-1- |
| | ((S)-1-methylpyrrolidin-2-yl)ethoxy)pyrimidin-2-yl)-1H-pyrazol-4-yl)-7- |
| | fluorobenzo[b]thiophene-3-carbonitrile |
| 313 | 2-amino-4-(3,5-dimethyl-1-(4-(1-methyl-6-oxo-1,6-dihydropyridin-3-yl)-6-((S)-1- |
| | ((R)-1-methylpyrrolidin-2-yl)ethoxy)pyrimidin-2-yl)-1H-pyrazol-4-yl)-7- |
| | fluorobenzo[b]thiophene-3-carbonitrile |
| 314 | 2-amino-4-(3,5-dimethyl-1-(4-(1-methyl-6-oxo-1,6-dihydropyridin-3-yl)-6-((R)-1- |
| | ((S)-1-methylpyrrolidin-2-yl)ethoxy)pyrimidin-2-yl)-1H-pyrazol-4-yl)-7- |
| | fluorobenzo[b]thiophene-3-carbonitrile |
| 315 | 2-amino-4-(3,5-dimethyl-1-(4-(1-methyl-6-oxo-1,6-dihydropyridin-3-yl)-6-((R)-1- |
| | ((R)-1-methylpyrrolidin-2-yl)ethoxy)pyrimidin-2-yl)-1H-pyrazol-4-yl)-7- |
| | fluorobenzo[b]thiophene-3-carbonitrile |
| 316 | 2-amino-(Rₐ)-4-(3,5-dimethyl-1-(4-(1-methyl-6-oxo-1,6-dihydropyridin-3-yl)-6-((S)- |
| | 1-((S)-1-methylpyrrolidin-2-yl)ethoxy)pyrimidin-2-yl)-1H-pyrazol-4-yl)-7- |
| | fluorobenzo[b]thiophene-3-carbonitrile |
| 317 | 2-amino-(Rₐ)-4-(3,5-dimethyl-1-(4-(1-methyl-6-oxo-1,6-dihydropyridin-3-yl)-6-((S)- |
| | 1-((R)-1-methylpyrrolidin-2-yl)ethoxy)pyrimidin-2-yl)-1H-pyrazol-4-yl)-7- |
| | fluorobenzo[b]thiophene-3-carbonitrile |
| 318 | 2-amino-(Rₐ)-4-(3,5-dimethyl-1-(4-(1-methyl-6-oxo-1,6-dihydropyridin-3-yl)-6-((R)- |
| | 1-((S)-1-methylpyrrolidin-2-yl)ethoxy)pyrimidin-2-yl)-1H-pyrazol-4-yl)-7- |
| | fluorobenzo[b]thiophene-3-carbonitrile |
| 319 | 2-amino-(Rₐ)-4-(3,5-dimethyl-1-(4-(1-methyl-6-oxo-1,6-dihydropyridin-3-yl)-6-((R)- |
| | 1-((R)-1-methylpyrrolidin-2-yl)ethoxy)pyrimidin-2-yl)-1H-pyrazol-4-yl)-7- |
| | fluorobenzo[b]thiophene-3-carbonitrile |
| 320 | 2-amino-(Sₐ)-4-(3,5-dimethyl-1-(4-(1-methyl-6-oxo-1,6-dihydropyridin-3-yl)-6-((S)- |
| | 1-((S)-1-methylpyrrolidin-2-yl)ethoxy)pyrimidin-2-yl)-1H-pyrazol-4-yl)-7- |
| | fluorobenzo[b]thiophene-3-carbonitrile |
| 321 | 2-amino-(Sₐ)-4-(3,5-dimethyl-1-(4-(1-methyl-6-oxo-1,6-dihydropyridin-3-yl)-6-((S)- |
| | 1-((R)-1-methylpyrrolidin-2-yl)ethoxy)pyrimidin-2-yl)-1H-pyrazol-4-yl)-7- |
| | fluorobenzo[b]thiophene-3-carbonitrile |
| 322 | 2-amino-(Sₐ)-4-(3,5-dimethyl-1-(4-(1-methyl-6-oxo-1,6-dihydropyridin-3-yl)-6-((R)- |
| | 1-((S)-1-methylpyrrolidin-2-yl)ethoxy)pyrimidin-2-yl)-1H-pyrazol-4-yl)-7- |
| | fluorobenzo[b]thiophene-3-carbonitrile |
| 323 | 2-amino-(Sₐ)-4-(3,5-dimethyl-1-(4-(1-methyl-6-oxo-1,6-dihydropyridin-3-yl)-6-((R)- |
| | 1-((R)-1-methylpyrrolidin-2-yl)ethoxy)pyrimidin-2-yl)-1H-pyrazol-4-yl)-7- |
| | fluorobenzo[b]thiophene-3-carbonitrile |
| 324 | 2-amino-4-(3,5-dimethyl-1-(4-((S)-1-((S)-1-methylpyrrolidin-2-yl)ethoxy)-6-(pyridin- |
| | 3-yl)pyrimidin-2-yl)-1H-pyrazol-4-yl)-7-fluorobenzo[b]thiophene-3-carbonitrile |
| 325 | 2-amino-4-(3,5-dimethyl-1-(4-((S)-1-((R)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 326 | 2-amino-4-(3,5-dimethyl-1-(4-((R)-1-((S)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 327 | 2-amino-4-(3,5-dimethyl-1-(4-((R)-1-((R)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 328 | 2-amino-(Rₐ)-4-(3,5-dimethyl-1-(4-((S)-1-((S)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 329 | 2-amino-(Rₐ)-4-(3,5-dimethyl-1-(4-((S)-1-((R)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 330 | 2-amino-(Rₐ)-4-(3,5-dimethyl-1-(4-((R)-1-((S)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 331 | 2-amino-(Rₐ)-4-(3,5-dimethyl-1-(4-((R)-1-((R)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 332 | 2-amino-(Sₐ)-4-(3,5-dimethyl-1-(4-((S)-1-((S)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 334 | 2-amino-(Sₐ)-4-(3,5-dimethyl-1-(4-((S)-1-((R)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 335 | 2-amino-(Sₐ)-4-(3,5-dimethyl-1-(4-((R)-1-((S)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 336 | 2-amino-(Sₐ)-4-(3,5-dimethyl-1-(4-((R)-1-((R)-1-methylpyrrolidin-2-yl)ethoxy)-6- |
| | (pyridin-3-yl)pyrimidin-2-yl)-1H-pyrazol-4-yl)-7-fluorobenzo[b]thiophene-3- |
| | carbonitrile |
| 337 | 2-amino-7-fluoro-4-(1-(4-(6-(2-hydroxypropan-2-yl)pyridin-3-yl)-6-((S)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 338 | 2-amino-7-fluoro-4-(1-(4-(6-(2-hydroxypropan-2-yl)pyridin-3-yl)-6-((S)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 339 | 2-amino-7-fluoro-4-(1-(4-(6-(2-hydroxypropan-2-yl)pyridin-3-yl)-6-((R)-1-((S)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 340 | 2-amino-7-fluoro-4-(1-(4-(6-(2-hydroxypropan-2-yl)pyridin-3-yl)-6-((R)-1-((R)-1- |
| | methylpyrrolidin-2-yl)ethoxy)pyrimidin-2-yl)-3,5-dimethyl-1H-pyrazol-4- |
| | yl)benzo[b]thiophene-3-carbonitrile |
| 341 | 2-amino-7-fluoro-(Rₐ)-4-(1-(4-(6-(2-hydroxypropan-2-yl)pyridin-3-yl)-6-((S)-1-((S)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 342 | 2-amino-7-fluoro-(Rₐ)-4-(1-(4-(6-(2-hydroxypropan-2-yl)pyridin-3-yl)-6-((S)-1-((R)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 343 | 2-amino-7-fluoro-(Rₐ)-4-(1-(4-(6-(2-hydroxypropan-2-yl)pyridin-3-yl)-6-((R)-1-((S)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 344 | 2-amino-7-fluoro-(Rₐ)-4-(1-(4-(6-(2-hydroxypropan-2-yl)pyridin-3-yl)-6-((R)-1-((R)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 345 | 2-amino-7-fluoro-(Sₐ)-4-(1-(4-(6-(2-hydroxypropan-2-yl)pyridin-3-yl)-6-((S)-1-((S)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 346 | 2-amino-7-fluoro-(Sₐ)-4-(1-(4-(6-(2-hydroxypropan-2-yl)pyridin-3-yl)-6-((S)-1-((R)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 347 | 2-amino-7-fluoro-(Sₐ)-4-(1-(4-(6-(2-hydroxypropan-2-yl)pyridin-3-yl)-6-((R)-1-((S)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 348 | 2-amino-7-fluoro-(Sₐ)-4-(1-(4-(6-(2-hydroxypropan-2-yl)pyridin-3-yl)-6-((R)-1-((R)- |
| | 1-methylpyrrolidin-2-yl)ethoxy)pyrimidin-2-yl)-3,5-dimethyl-1H-pyrazol-4- |
| | yl)benzo[b]thiophene-3-carbonitrile |
| 349 | 5-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N,N- |
| | dimethylpicolinamide |
| 350 | 5-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N,N- |
| | dimethylpicolinamide |
| 351 | 5-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N,N- |
| | dimethylpicolinamide |
| 352 | 5-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N,N- |
| | dimethylpicolinamide |
| 353 | 5-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N,N- |
| | dimethylpicolinamide |
| 354 | 5-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N,N- |
| | dimethylpicolinamide |
| 355 | 5-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N,N- |
| | dimethylpicolinamide |
| 356 | 5-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N,N- |
| | dimethylpicolinamide |
| 357 | 5-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N,N- |
| | dimethylpicolinamide |
| 358 | 5-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N,N- |
| | dimethylpicolinamide |
| 359 | 5-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N,N- |
| | dimethylpicolinamide |
| 360 | 5-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N,N- |
| | dimethylpicolinamide |
| 361 | 5-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N- |
| | methylpicolinamide |
| 362 | 5-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N- |
| | methylpicolinamide |
| 363 | 5-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N- |
| | methylpicolinamide |
| 364 | 5-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N- |
| | methylpicolinamide |
| 365 | 5-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N- |
| | methylpicolinamide |
| 366 | 5-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N- |
| | methylpicolinamide |
| 367 | 5-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N- |
| | methylpicolinamide |
| 368 | 5-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N- |
| | methylpicolinamide |
| 369 | 5-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N- |
| | methylpicolinamide |
| 370 | 5-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N- |
| | methylpicolinamide |
| 371 | 5-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N- |
| | methylpicolinamide |
| 372 | 5-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N- |
| | methylpicolinamide |
| 373 | 2-amino-4-(3,5-dimethyl-1-(6-((S)-1-((S)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 374 | 2-amino-4-(3,5-dimethyl-1-(6-((S)-1-((R)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 375 | 2-amino-4-(3,5-dimethyl-1-(6-((R)-1-((S)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 376 | 2-amino-4-(3,5-dimethyl-1-(6-((R)-1-((R)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 377 | 2-amino-(Rₐ)-4-(3,5-dimethyl-1-(6-((S)-1-((S)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 378 | 2-amino-(Rₐ)-4-(3,5-dimethyl-1-(6-((S)-1-((R)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 379 | 2-amino-(Rₐ)-4-(3,5-dimethyl-1-(6-((R)-1-((S)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 380 | 2-amino-(Rₐ)-4-(3,5-dimethyl-1-(6-((R)-1-((R)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 381 | 2-amino-(Sₐ)-4-(3,5-dimethyl-1-(6-((S)-1-((S)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 382 | 2-amino-(Sₐ)-4-(3,5-dimethyl-1-(6-((S)-1-((R)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 383 | 2-amino-(Sₐ)-4-(3,5-dimethyl-1-(6-((R)-1-((S)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 384 | 2-amino-(Sₐ)-4-(3,5-dimethyl-1-(6-((R)-1-((R)-1-methylpyrrolidin-2-yl)ethoxy)-[4,5'- |
| | bipyrimidin]-2-yl)-1H-pyrazol-4-yl)-7-fluorobenzo[b]thiophene-3-carbonitrile |
| 385 | 2-amino-4-(3,5-dimethyl-1-(4-((S)-1-((S)-1-methylpyrrolidin-2-yl)ethoxy)-6-(pyridin- |
| | 4-yl)pyrimidin-2-yl)-1H-pyrazol-4-yl)-7-fluorobenzo[b]thiophene-3-carbonitrile |
| 386 | 2-amino-4-(3,5-dimethyl-1-(4-((S)-1-((R)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 387 | 2-amino-4-(3,5-dimethyl-1-(4-((R)-1-((S)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 388 | 2-amino-4-(3,5-dimethyl-1-(4-((R)-1-((R)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 389 | 2-amino-(Rₐ)-4-(3,5-dimethyl-1-(4-((S)-1-((S)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 390 | 2-amino-(Rₐ)-4-(3,5-dimethyl-1-(4-((S)-1-((R)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 391 | 2-amino-(Rₐ)-4-(3,5-dimethyl-1-(4-((R)-1-((S)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 392 | 2-amino-(Rₐ)-4-(3,5-dimethyl-1-(4-((R)-1-((R)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 393 | 2-amino-(Sₐ)-4-(3,5-dimethyl-1-(4-((S)-1-((S)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 394 | 2-amino-(Sₐ)-4-(3,5-dimethyl-1-(4-((S)-1-((R)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 395 | 2-amino-(Sₐ)-4-(3,5-dimethyl-1-(4-((R)-1-((S)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 396 | 2-amino-(Sₐ)-4-(3,5-dimethyl-1-(4-((R)-1-((R)-1-methylpyrrolidin-2-yl)ethoxy)-6- |
| | (pyridin-4-yl)pyrimidin-2-yl)-1H-pyrazol-4-yl)-7-fluorobenzo[b]thiophene-3- |
| | carbonitrile |
| 397 | 1-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-1H-pyrazole-3- |
| | carboxamide |
| 398 | 1-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-1H-pyrazole-3- |
| | carboxamide |
| 399 | 1-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-1H-pyrazole-3- |
| | carboxamide |
| 400 | 1-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-1H-pyrazole-3- |
| | carboxamide |
| 401 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-1H- |
| | pyrazole-3-carboxamide |
| 402 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-1H- |
| | pyrazole-3-carboxamide |
| 403 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-1H- |
| | pyrazole-3-carboxamide |
| 404 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-1H- |
| | pyrazole-3-carboxamide |
| 405 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-1H- |
| | pyrazole-3-carboxamide |
| 406 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-1H- |
| | pyrazole-3-carboxamide |
| 407 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-1H- |
| | pyrazole-3-carboxamide |
| 408 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-1H- |
| | pyrazole-3-carboxamide |
| 409 | 1-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N-methyl-1H- |
| | pyrazole-3-carboxamide |
| 410 | 1-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N-methyl-1H- |
| | pyrazole-3-carboxamide |
| 411 | 1-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N-methyl-1H- |
| | pyrazole-3-carboxamide |
| 412 | 1-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N-methyl-1H- |
| | pyrazole-3-carboxamide |
| 413 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N-methyl- |
| | 1H-pyrazole-3-carboxamide |
| 414 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N-methyl- |
| | 1H-pyrazole-3-carboxamide |
| 415 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N-methyl- |
| | 1H-pyrazole-3-carboxamide |
| 416 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N-methyl- |
| | 1H-pyrazole-3-carboxamide |
| 417 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N-methyl- |
| | 1H-pyrazole-3-carboxamide |
| 418 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N-methyl- |
| | 1H-pyrazole-3-carboxamide |
| 419 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N-methyl- |
| | 1H-pyrazole-3-carboxamide |
| 420 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N-methyl- |
| | 1H-pyrazole-3-carboxamide |
| 421 | 1-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N,N-dimethyl-1H- |
| | pyrazole-3-carboxamide |
| 422 | 1-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N,N-dimethyl-1H- |
| | pyrazole-3-carboxamide |
| 423 | 1-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N,N-dimethyl-1H- |
| | pyrazole-3-carboxamide |
| 424 | 1-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N,N-dimethyl-1H- |
| | pyrazole-3-carboxamide |
| 425 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N,N- |
| | dimethyl-1H-pyrazole-3-carboxamide |
| 426 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N,N- |
| | dimethyl-1H-pyrazole-3-carboxamide |
| 427 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N,N- |
| | dimethyl-1H-pyrazole-3-carboxamide |
| 428 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N,N- |
| | dimethyl-1H-pyrazole-3-carboxamide |
| 429 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N,N- |
| | dimethyl-1H-pyrazole-3-carboxamide |
| 430 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N,N- |
| | dimethyl-1H-pyrazole-3-carboxamide |
| 431 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N,N- |
| | dimethyl-1H-pyrazole-3-carboxamide |
| 432 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N,N- |
| | dimethyl-1H-pyrazole-3-carboxamide |
| 433 | 1-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N-methyl-1H- |
| | pyrazole-4-carboxamide |
| 434 | 1-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N-methyl-1H- |
| | pyrazole-4-carboxamide |
| 435 | 1-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N-methyl-1H- |
| | pyrazole-4-carboxamide |
| 436 | 1-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N-methyl-1H- |
| | pyrazole-4-carboxamide |
| 437 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N-methyl- |
| | 1H-pyrazole-4-carboxamide |
| 438 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N-methyl- |
| | 1H-pyrazole-4-carboxamide |
| 439 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N-methyl- |
| | 1H-pyrazole-4-carboxamide |
| 440 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N-methyl- |
| | 1H-pyrazole-4-carboxamide |
| 441 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N-methyl- |
| | 1H-pyrazole-4-carboxamide |
| 442 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N-methyl- |
| | 1H-pyrazole-4-carboxamide |
| 443 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N-methyl- |
| | 1H-pyrazole-4-carboxamide |
| 444 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N-methyl- |
| | 1H-pyrazole-4-carboxamide |
| 445 | 2-amino-4-(3,5-dimethyl-1-(4-((S)-1-((S)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 446 | 2-amino-4-(3,5-dimethyl-1-(4-((S)-1-((R)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 447 | 2-amino-4-(3,5-dimethyl-1-(4-((R)-1-((S)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 448 | 2-amino-4-(3,5-dimethyl-1-(4-((R)-1-((R)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 449 | 2-amino-(Rₐ)-4-(3,5-dimethyl-1-(4-((S)-1-((S)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 450 | 2-amino-(Rₐ)-4-(3,5-dimethyl-1-(4-((S)-1-((R)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 451 | 2-amino-(Rₐ)-4-(3,5-dimethyl-1-(4-((R)-1-((S)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 452 | 2-amino-(Rₐ)-4-(3,5-dimethyl-1-(4-((R)-1-((R)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 453 | 2-amino-(Sₐ)-4-(3,5-dimethyl-1-(4-((S)-1-((S)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 454 | 2-amino-(Sₐ)-4-(3,5-dimethyl-1-(4-((S)-1-((R)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 455 | 2-amino-(Sₐ)-4-(3,5-dimethyl-1-(4-((R)-1-((S)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 456 | 2-amino-(Sₐ)-4-(3,5-dimethyl-1-(4-((R)-1-((R)-1-methylpyrrolidin-2-yl)ethoxy)-6- |
| | (pyrimidin-5-yloxy)pyrimidin-2-yl)-1H-pyrazol-4-yl)-7-fluorobenzo[b]thiophene-3- |
| | carbonitrile |
| 457 | 4-(1-(4-((1-acetylpiperidin-4-yl)oxy)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 458 | 4-(1-(4-((1-acetylpiperidin-4-yl)oxy)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 459 | 4-(1-(4-((1-acetylpiperidin-4-yl)oxy)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 460 | 4-(1-(4-((1-acetylpiperidin-4-yl)oxy)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 461 | (Rₐ)-4-(1-(4-((1-acetylpiperidin-4-yl)oxy)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 462 | (Rₐ)-4-(1-(4-((1-acetylpiperidin-4-yl)oxy)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 463 | (Rₐ)-4-(1-(4-((1-acetylpiperidin-4-yl)oxy)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 464 | (Rₐ)-4-(1-(4-((1-acetylpiperidin-4-yl)oxy)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 465 | (Sₐ)-4-(1-(4-((1-acetylpiperidin-4-yl)oxy)-6-((S)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 466 | (Sₐ)-4-(1-(4-((1-acetylpiperidin-4-yl)oxy)-6-((S)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 467 | (Sₐ)-4-(1-(4-((1-acetylpiperidin-4-yl)oxy)-6-((R)-1-((S)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 468 | (Sₐ)-4-(1-(4-((1-acetylpiperidin-4-yl)oxy)-6-((R)-1-((R)-1-methylpyrrolidin-2- |
| | yl)ethoxy)pyrimidin-2-yl)-3,5-dimethyl-1H-pyrazol-4-yl)-2-amino-7- |
| | fluorobenzo[b]thiophene-3-carbonitrile |
| 469 | 2-amino-4-(3,5-dimethyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 470 | 2-amino-4-(3,5-dimethyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 471 | 2-amino-4-(3,5-dimethyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 472 | 2-amino-4-(3,5-dimethyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 473 | 2-amino-(Rₐ)-4-(3,5-dimethyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 474 | 2-amino-(Rₐ)-4-(3,5-dimethyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 475 | 2-amino-(Rₐ)-4-(3,5-dimethyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 476 | 2-amino-(Rₐ)-4-(3,5-dimethyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 477 | 2-amino-(Sₐ)-4-(3,5-dimethyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 478 | 2-amino-(Sₐ)-4-(3,5-dimethyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 479 | 2-amino-(Sₐ)-4-(3,5-dimethyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 480 | 2-amino-(Sₐ)-4-(3,5-dimethyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 481 | 2-amino-4-(3,5-dimethyl-1-(2-((S)-1-((S)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 482 | 2-amino-4-(3,5-dimethyl-1-(2-((S)-1-((R)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 483 | 2-amino-4-(3,5-dimethyl-1-(2-((R)-1-((S)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 484 | 2-amino-4-(3,5-dimethyl-1-(2-((R)-1-((R)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 485 | 2-amino-(Rₐ)-4-(3,5-dimethyl-1-(2-((S)-1-((S)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 486 | 2-amino-(Rₐ)-4-(3,5-dimethyl-1-(2-((S)-1-((R)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 487 | 2-amino-(Rₐ)-4-(3,5-dimethyl-1-(2-((R)-1-((S)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 488 | 2-amino-(Rₐ)-4-(3,5-dimethyl-1-(2-((R)-1-((R)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 489 | 2-amino-(Sₐ)-4-(3,5-dimethyl-1-(2-((S)-1-((S)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 490 | 2-amino-(Sₐ)-4-(3,5-dimethyl-1-(2-((S)-1-((R)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 491 | 2-amino-(Sₐ)-4-(3,5-dimethyl-1-(2-((R)-1-((S)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 492 | 2-amino-(Sₐ)-4-(3,5-dimethyl-1-(2-((R)-1-((R)-1-methylpyrrolidin-2-yl)ethoxy)-6-(1- |
| | (oxetan-3-yl)-1H-pyrazol-4-yl)pyrimidin-4-yl)-1H-pyrazol-4-yl)-7- |
| | fluorobenzo[b]thiophene-3-carbonitrile |
| 493 | 2-amino-4-(1-(6-(1-(difluoromethyl)-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 494 | 2-amino-4-(1-(6-(1-(difluoromethyl)-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 495 | 2-amino-4-(1-(6-(1-(difluoromethyl)-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 496 | 2-amino-4-(1-(6-(1-(difluoromethyl)-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 497 | 2-amino-(Rₐ)-4-(1-(6-(1-(difluoromethyl)-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 498 | 2-amino-(Rₐ)-4-(1-(6-(1-(difluoromethyl)-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 499 | 2-amino-(Rₐ)-4-(1-(6-(1-(difluoromethyl)-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 500 | 2-amino-(Rₐ)-4-(1-(6-(1-(difluoromethyl)-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 501 | 2-amino-(Sₐ)-4-(1-(6-(1-(difluoromethyl)-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 502 | 2-amino-(Sₐ)-4-(1-(6-(1-(difluoromethyl)-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 503 | 2-amino-(Sₐ)-4-(1-(6-(1-(difluoromethyl)-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 504 | 2-amino-(Sₐ)-4-(1-(6-(1-(difluoromethyl)-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 505 | 2-amino-4-(3,5-dimethyl-1-(2-((S)-1-((S)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 506 | 2-amino-4-(3,5-dimethyl-1-(2-((S)-1-((R)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 507 | 2-amino-4-(3,5-dimethyl-1-(2-((R)-1-((S)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 508 | 2-amino-4-(3,5-dimethyl-1-(2-((R)-1-((R)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 509 | 2-amino-(Rₐ)-4-(3,5-dimethyl-1-(2-((S)-1-((S)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 510 | 2-amino-(Rₐ)-4-(3,5-dimethyl-1-(2-((S)-1-((R)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 511 | 2-amino-(Rₐ)-4-(3,5-dimethyl-1-(2-((R)-1-((S)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 512 | 2-amino-(Rₐ)-4-(3,5-dimethyl-1-(2-((R)-1-((R)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 513 | 2-amino-(Sₐ)-4-(3,5-dimethyl-1-(2-((S)-1-((S)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 514 | 2-amino-(Sₐ)-4-(3,5-dimethyl-1-(2-((S)-1-((R)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 515 | 2-amino-(Sₐ)-4-(3,5-dimethyl-1-(2-((R)-1-((S)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 516 | 2-amino-(Sₐ)-4-(3,5-dimethyl-1-(2-((R)-1-((R)-1-methylpyrrolidin-2-yl)ethoxy)-6-(1- |
| | (pyridin-3-yl)-1H-pyrazol-4-yl)pyrimidin-4-yl)-1H-pyrazol-4-yl)-7- |
| | fluorobenzo[b]thiophene-3-carbonitrile |
| 517 | 2-amino-4-(1-(6-(1-benzyl-1H-pyrazol-4-yl)-2-((S)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 518 | 2-amino-4-(1-(6-(1-benzyl-1H-pyrazol-4-yl)-2-((S)-1-((R)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 519 | 2-amino-4-(1-(6-(1-benzyl-1H-pyrazol-4-yl)-2-((R)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 520 | 2-amino-4-(1-(6-(1-benzyl-1H-pyrazol-4-yl)-2-((R)-1-((R)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 521 | 2-amino-(Rₐ)-4-(1-(6-(1-benzyl-1H-pyrazol-4-yl)-2-((S)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 522 | 2-amino-(Rₐ)-4-(1-(6-(1-benzyl-1H-pyrazol-4-yl)-2-((S)-1-((R)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 523 | 2-amino-(Rₐ)-4-(1-(6-(1-benzyl-1H-pyrazol-4-yl)-2-((R)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 524 | 2-amino-(Rₐ)-4-(1-(6-(1-benzyl-1H-pyrazol-4-yl)-2-((R)-1-((R)-1-methylpyrrolidin- |
| | 2-yl)ethoxy)pyrimidin-4-yl)-3,5-dimethyl-1H-pyrazol-4-yl)-7- |
| | fluorobenzo[b]thiophene-3-carbonitrile |
| 525 | 2-amino-(Sₐ)-4-(1-(6-(1-benzyl-1H-pyrazol-4-yl)-2-((S)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 526 | 2-amino-(Sₐ)-4-(1-(6-(1-benzyl-1H-pyrazol-4-yl)-2-((S)-1-((R)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 527 | 2-amino-(Sₐ)-4-(1-(6-(1-benzyl-1H-pyrazol-4-yl)-2-((R)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 528 | 2-amino-(Sₐ)-4-(1-(6-(1-benzyl-1H-pyrazol-4-yl)-2-((R)-1-((R)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 42529 | 2-amino-4-(3,5-dimethyl-1-(2-((S)-1-((S)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 530 | 2-amino-4-(3,5-dimethyl-1-(2-((S)-1-((R)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 531 | 2-amino-4-(3,5-dimethyl-1-(2-((R)-1-((S)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 532 | 2-amino-4-(3,5-dimethyl-1-(2-((R)-1-((R)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 533 | 2-amino-(Rₐ)-4-(3,5-dimethyl-1-(2-((S)-1-((S)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 534 | 2-amino-(Rₐ)-4-(3,5-dimethyl-1-(2-((S)-1-((R)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 535 | 2-amino-(Rₐ)-4-(3,5-dimethyl-1-(2-((R)-1-((S)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 536 | 2-amino-(Rₐ)-4-(3,5-dimethyl-1-(2-((R)-1-((R)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 537 | 2-amino-(Sₐ)-4-(3,5-dimethyl-1-(2-((S)-1-((S)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 538 | 2-amino-(Sₐ)-4-(3,5-dimethyl-1-(2-((S)-1-((R)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 539 | 2-amino-(Sₐ)-4-(3,5-dimethyl-1-(2-((R)-1-((S)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 540 | 2-amino-(Sₐ)-4-(3,5-dimethyl-1-(2-((R)-1-((R)-1-methylpyrrolidin-2-yl)ethoxy)-6-(1- |
| | (pyridin-3-ylmethyl)-1H-pyrazol-4-yl)pyrimidin-4-yl)-1H-pyrazol-4-yl)-7- |
| | fluorobenzo[b]thiophene-3-carbonitrile |
| 541 | 2-amino-7-fluoro-4-(1-(6-(5-fluoro-1-methyl-1H-pyrazol-4-yl)-2-((S)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 542 | 2-amino-7-fluoro-4-(1-(6-(5-fluoro-1-methyl-1H-pyrazol-4-yl)-2-((S)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 543 | 2-amino-7-fluoro-4-(1-(6-(5-fluoro-1-methyl-1H-pyrazol-4-yl)-2-((R)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 544 | 2-amino-7-fluoro-4-(1-(6-(5-fluoro-1-methyl-1H-pyrazol-4-yl)-2-((R)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 545 | 2-amino-7-fluoro-(Rₐ)-4-(1-(6-(5-fluoro-1-methyl-1H-pyrazol-4-yl)-2-((S)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 546 | 2-amino-7-fluoro-(Rₐ)-4-(1-(6-(5-fluoro-1-methyl-1H-pyrazol-4-yl)-2-((S)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 547 | 2-amino-7-fluoro-(Rₐ)-4-(1-(6-(5-fluoro-1-methyl-1H-pyrazol-4-yl)-2-((R)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 548 | 2-amino-7-fluoro-(Rₐ)-4-(1-(6-(5-fluoro-1-methyl-1H-pyrazol-4-yl)-2-((R)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 549 | 2-amino-7-fluoro-(Sₐ)-4-(1-(6-(5-fluoro-1-methyl-1H-pyrazol-4-yl)-2-((S)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 550 | 2-amino-7-fluoro-(Sₐ)-4-(1-(6-(5-fluoro-1-methyl-1H-pyrazol-4-yl)-2-((S)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 551 | 2-amino-7-fluoro-(Sₐ)-4-(1-(6-(5-fluoro-1-methyl-1H-pyrazol-4-yl)-2-((R)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 552 | 2-amino-7-fluoro-(Sₐ)-4-(1-(6-(5-fluoro-1-methyl-1H-pyrazol-4-yl)-2-((R)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 553 | 2-amino-4-(1-(6-(1,3-dimethyl-1H-pyrazol-4-yl)-2-((S)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 554 | 2-amino-4-(1-(6-(1,3-dimethyl-1H-pyrazol-4-yl)-2-((S)-1-((R)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 555 | 2-amino-4-(1-(6-(1,3-dimethyl-1H-pyrazol-4-yl)-2-((R)-1-((S)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 556 | 2-amino-4-(1-(6-(1,3-dimethyl-1H-pyrazol-4-yl)-2-((R)-1-((R)-1-methylpyrrolidin-2- |
| | yl)ethoxy)pyrimidin-4-yl)-3,5-dimethyl-1H-pyrazol-4-yl)-7-fluorobenzo[b]thiophene- |
| | 3-carbonitrile |
| 557 | 2-amino-(Rₐ)-4-(1-(6-(1,3-dimethyl-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 558 | 2-amino-(Rₐ)-4-(1-(6-(1,3-dimethyl-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 559 | 2-amino-(Rₐ)-4-(1-(6-(1,3-dimethyl-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 560 | 2-amino-(Rₐ)-4-(1-(6-(1,3-dimethyl-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 561 | 2-amino-(Sₐ)-4-(1-(6-(1,3-dimethyl-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 562 | 2-amino-(Sₐ)-4-(1-(6-(1,3-dimethyl-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 563 | 2-amino-(Sₐ)-4-(1-(6-(1,3-dimethyl-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 564 | 2-amino-(Sₐ)-4-(1-(6-(1,3-dimethyl-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 565 | 4-(6-(4-(2-amino-3-cyano-7-fluorobenzo[b]thiophen-4-yl)-3,5-dimethyl-1H-pyrazol- |
| | 1-yl)-2-((S)-1-((S)-1-methylpyrrolidin-2-yl)ethoxy)pyrimidin-4-yl)-N- |
| | methylpicolinamide |
| 566 | 4-(6-(4-(2-amino-3-cyano-7-fluorobenzo[b]thiophen-4-yl)-3,5-dimethyl-1H-pyrazol- |
| | 1-yl)-2-((S)-1-((R)-1-methylpyrrolidin-2-yl)ethoxy)pyrimidin-4-yl)-N- |
| | methylpicolinamide |
| 567 | 4-(6-(4-(2-amino-3-cyano-7-fluorobenzo[b]thiophen-4-yl)-3,5-dimethyl-1H-pyrazol- |
| | 1-yl)-2-((R)-1-((S)-1-methylpyrrolidin-2-yl)ethoxy)pyrimidin-4-yl)-N- |
| | methylpicolinamide |
| 568 | 4-(6-(4-(2-amino-3-cyano-7-fluorobenzo[b]thiophen-4-yl)-3,5-dimethyl-1H-pyrazol- |
| | 1-yl)-2-((R)-1-((R)-1-methylpyrrolidin-2-yl)ethoxy)pyrimidin-4-yl)-N- |
| | methylpicolinamide |
| 569 | 4-(6-((Rₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-N- |
| | methylpicolinamide |
| 570 | 4-(6-((Rₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-N- |
| | methylpicolinamide |
| 571 | 4-(6-((Rₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-N- |
| | methylpicolinamide |
| 572 | 4-(6-((Rₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-N- |
| | methylpicolinamide |
| 573 | 4-(6-((Sₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-N- |
| | methylpicolinamide |
| 574 | 4-(6-((Sₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-N- |
| | methylpicolinamide |
| 575 | 4-(6-((Sₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-N- |
| | methylpicolinamide |
| 576 | 4-(6-((Sₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-N- |
| | methylpicolinamide |
| 577 | 2-amino-4-(1-(6-(1-(cyanomethyl)-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 578 | 2-amino-4-(1-(6-(1-(cyanomethyl)-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 579 | 2-amino-4-(1-(6-(1-(cyanomethyl)-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 580 | 2-amino-4-(1-(6-(1-(cyanomethyl)-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 581 | 2-amino-(Rₐ)-4-(1-(6-(1-(cyanomethyl)-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 582 | 2-amino-(Rₐ)-4-(1-(6-(1-(cyanomethyl)-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 583 | 2-amino-(Rₐ)-4-(1-(6-(1-(cyanomethyl)-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 584 | 2-amino-(Rₐ)-4-(1-(6-(1-(cyanomethyl)-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 585 | 2-amino-(Sₐ)-4-(1-(6-(1-(cyanomethyl)-1H-pyrazol-4-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 586 | 2-amino-(Sₐ)-4-(1-(6-(1-(cyanomethyl)-1H-pyrazol-4-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 587 | 2-amino-(Sₐ)-4-(1-(6-(1-(cyanomethyl)-1H-pyrazol-4-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 588 | 2-amino-(Sₐ)-4-(1-(6-(1-(cyanomethyl)-1H-pyrazol-4-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 589 | 2-amino-4-(1-(6-(4-(cyclopropanecarbonyl)piperazin-1-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 590 | 2-amino-4-(1-(6-(4-(cyclopropanecarbonyl)piperazin-1-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 591 | 2-amino-4-(1-(6-(4-(cyclopropanecarbonyl)piperazin-1-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 592 | 2-amino-4-(1-(6-(4-(cyclopropanecarbonyl)piperazin-1-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 593 | 2-amino-(Rₐ)-4-(1-(6-(4-(cyclopropanecarbonyl)piperazin-1-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 594 | 2-amino-(Rₐ)-4-(1-(6-(4-(cyclopropanecarbonyl)piperazin-1-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 595 | 2-amino-(Rₐ)-4-(1-(6-(4-(cyclopropanecarbonyl)piperazin-1-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 596 | 2-amino-(Rₐ)-4-(1-(6-(4-(cyclopropanecarbonyl)piperazin-1-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 597 | 2-amino-(Sₐ)-4-(1-(6-(4-(cyclopropanecarbonyl)piperazin-1-yl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 598 | 2-amino-(Sₐ)-4-(1-(6-(4-(cyclopropanecarbonyl)piperazin-1-yl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 599 | 2-amino-(Sₐ)-4-(1-(6-(4-(cyclopropanecarbonyl)piperazin-1-yl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 600 | 2-amino-(Sₐ)-4-(1-(6-(4-(cyclopropanecarbonyl)piperazin-1-yl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 601 | 4-(1-(6-(4-acetylpiperazin-1-yl)-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 602 | 4-(1-(6-(4-acetylpiperazin-1-yl)-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 603 | 4-(1-(6-(4-acetylpiperazin-1-yl)-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 604 | 4-(1-(6-(4-acetylpiperazin-1-yl)-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 605 | (Rₐ)-4-(1-(6-(4-acetylpiperazin-1-yl)-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 606 | (Rₐ)-4-(1-(6-(4-acetylpiperazin-1-yl)-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 607 | (Rₐ)-4-(1-(6-(4-acetylpiperazin-1-yl)-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 608 | (Rₐ)-4-(1-(6-(4-acetylpiperazin-1-yl)-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 609 | (Sₐ)-4-(1-(6-(4-acetylpiperazin-1-yl)-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 610 | (Sₐ)-4-(1-(6-(4-acetylpiperazin-1-yl)-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 611 | (Sₐ)-4-(1-(6-(4-acetylpiperazin-1-yl)-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 612 | (Sₐ)-4-(1-(6-(4-acetylpiperazin-1-yl)-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 613 | 5-(6-(4-(2-amino-3-cyano-7-fluorobenzo[b]thiophen-4-yl)-3,5-dimethyl-1H-pyrazol- |
| | 1-yl)-2-((S)-1-((S)-1-methylpyrrolidin-2-yl)ethoxy)pyrimidin-4-yl)-N,N-dimethyl- |
| | 5,6,7,8-tetrahydro-4H-pyrazolo[1,5-a][1,4]diazepine-2-carboxamide |
| 614 | 5-(6-(4-(2-amino-3-cyano-7-fluorobenzo[b]thiophen-4-yl)-3,5-dimethyl-1H-pyrazol- |
| | 1-yl)-2-((S)-1-((R)-1-methylpyrrolidin-2-yl)ethoxy)pyrimidin-4-yl)-N,N-dimethyl- |
| | 5,6,7,8-tetrahydro-4H-pyrazolo[1,5-a][1,4]diazepine-2-carboxamide |
| 615 | 5-(6-(4-(2-amino-3-cyano-7-fluorobenzo[b]thiophen-4-yl)-3,5-dimethyl-1H-pyrazol- |
| | 1-yl)-2-((R)-1-((S)-1-methylpyrrolidin-2-yl)ethoxy)pyrimidin-4-yl)-N,N-dimethyl- |
| | 5,6,7,8-tetrahydro-4H-pyrazolo[1,5-a][1,4]diazepine-2-carboxamide |
| 616 | 5-(6-(4-(2-amino-3-cyano-7-fluorobenzo[b]thiophen-4-yl)-3,5-dimethyl-1H-pyrazol- |
| | 1-yl)-2-((R)-1-((R)-1-methylpyrrolidin-2-yl)ethoxy)pyrimidin-4-yl)-N,N-dimethyl- |
| | 5,6,7,8-tetrahydro-4H-pyrazolo[1,5-a][1,4]diazepine-2-carboxamide |
| 617 | 5-(6-((Rₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 618 | 5-(6-((Rₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 619 | 5-(6-((Rₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 620 | 5-(6-((Rₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 621 | 5-(6-((Sₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 622 | 5-(6-((Sₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 623 | 5-(6-((Sₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 624 | 5-(6-((Sₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-N,N- |
| | dimethyl-5,6,7,8-tetrahydro-4H-pyrazolo[1,5-a][1,4]diazepine-2-carboxamide |
| 625 | 1-(6-(4-(2-amino-3-cyano-7-fluorobenzo[b]thiophen-4-yl)-3,5-dimethyl-1H-pyrazol- |
| | 1-yl)-2-((S)-1-((S)-1-methylpyrrolidin-2-yl)ethoxy)pyrimidin-4-yl)-1H-pyrazole-3- |
| | carboxamide |
| 626 | 1-(6-(4-(2-amino-3-cyano-7-fluorobenzo[b]thiophen-4-yl)-3,5-dimethyl-1H-pyrazol- |
| | 1-yl)-2-((S)-1-((R)-1-methylpyrrolidin-2-yl)ethoxy)pyrimidin-4-yl)-1H-pyrazole-3- |
| | carboxamide |
| 627 | 1-(6-(4-(2-amino-3-cyano-7-fluorobenzo[b]thiophen-4-yl)-3,5-dimethyl-1H-pyrazol- |
| | 1-yl)-2-((R)-1-((S)-1-methylpyrrolidin-2-yl)ethoxy)pyrimidin-4-yl)-1H-pyrazole-3- |
| | carboxamide |
| 628 | 1-(6-(4-(2-amino-3-cyano-7-fluorobenzo[b]thiophen-4-yl)-3,5-dimethyl-1H-pyrazol- |
| | 1-yl)-2-((R)-1-((R)-1-methylpyrrolidin-2-yl)ethoxy)pyrimidin-4-yl)-1H-pyrazole-3- |
| | carboxamide |
| 629 | 1-(6-((Rₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-1H- |
| | pyrazole-3-carboxamide |
| 630 | 1-(6-((Rₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-1H- |
| | pyrazole-3-carboxamide |
| 631 | 1-(6-((Rₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-1H- |
| | pyrazole-3-carboxamide |
| 632 | 1-(6-((Rₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-1H- |
| | pyrazole-3-carboxamide |
| 633 | 1-(6-((Sₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-1H- |
| | pyrazole-3-carboxamide |
| 634 | 1-(6-((Sₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-1H- |
| | pyrazole-3-carboxamide |
| 635 | 1-(6-((Sₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-1H- |
| | pyrazole-3-carboxamide |
| 636 | 1-(6-((Sₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-1H- |
| | pyrazole-3-carboxamide |
| 637 | 4-(1-(6-(4-acetylpiperazin-1-yl)-5-fluoro-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 638 | 4-(1-(6-(4-acetylpiperazin-1-yl)-5-fluoro-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 639 | 4-(1-(6-(4-acetylpiperazin-1-yl)-5-fluoro-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 640 | 4-(1-(6-(4-acetylpiperazin-1-yl)-5-fluoro-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 641 | (Rₐ)-4-(1-(6-(4-acetylpiperazin-1-yl)-5-fluoro-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 642 | (Rₐ)-4-(1-(6-(4-acetylpiperazin-1-yl)-5-fluoro-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 643 | (Rₐ)-4-(1-(6-(4-acetylpiperazin-1-yl)-5-fluoro-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 644 | (Rₐ)-4-(1-(6-(4-acetylpiperazin-1-yl)-5-fluoro-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 645 | (Sₐ)-4-(1-(6-(4-acetylpiperazin-1-yl)-5-fluoro-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 646 | (Sₐ)-4-(1-(6-(4-acetylpiperazin-1-yl)-5-fluoro-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 647 | (Sₐ)-4-(1-(6-(4-acetylpiperazin-1-yl)-5-fluoro-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 648 | (Sₐ)-4-(1-(6-(4-acetylpiperazin-1-yl)-5-fluoro-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 649 | 4-(4-acetylpiperazin-1-yl)-6-(4-(2-amino-3-cyano-7-fluorobenzo[b]thiophen-4-yl)- |
| | 3,5-dimethyl-1H-pyrazol-1-yl)-2-((S)-1-((S)-1-methylpyrrolidin-2- |
| | yl)ethoxy)pyrimidine-5-carbonitrile |
| 650 | 4-(4-acetylpiperazin-1-yl)-6-(4-(2-amino-3-cyano-7-fluorobenzo[b]thiophen-4-yl)- |
| | 3,5-dimethyl-1H-pyrazol-1-yl)-2-((S)-1-((R)-1-methylpyrrolidin-2- |
| | yl)ethoxy)pyrimidine-5-carbonitrile |
| 651 | 4-(4-acetylpiperazin-1-yl)-6-(4-(2-amino-3-cyano-7-fluorobenzo[b]thiophen-4-yl)- |
| | 3,5-dimethyl-1H-pyrazol-1-yl)-2-((R)-1-((S)-1-methylpyrrolidin-2- |
| | yl)ethoxy)pyrimidine-5-carbonitrile |
| 652 | 4-(4-acetylpiperazin-1-yl)-6-(4-(2-amino-3-cyano-7-fluorobenzo[b]thiophen-4-yl)- |
| | 3,5-dimethyl-1H-pyrazol-1-yl)-2-((R)-1-((R)-1-methylpyrrolidin-2- |
| | yl)ethoxy)pyrimidine-5-carbonitrile |
| 653 | 4-(4-acetylpiperazin-1-yl)-6-((Rₐ)-4-(2-amino-3-cyano-7-fluorobenzo[b]thiophen-4- |
| | yl)-3,5-dimethyl-1H-pyrazol-1-yl)-2-((S)-1-((S)-1-methylpyrrolidin-2- |
| | yl)ethoxy)pyrimidine-5-carbonitrile |
| 654 | 4-(4-acetylpiperazin-1-yl)-6-((Rₐ)-4-(2-amino-3-cyano-7-fluorobenzo[b]thiophen-4- |
| | yl)-3,5-dimethyl-1H-pyrazol-1-yl)-2-((S)-1-((R)-1-methylpyrrolidin-2- |
| | yl)ethoxy)pyrimidine-5-carbonitrile |
| 655 | 4-(4-acetylpiperazin-1-yl)-6-((Rₐ)-4-(2-amino-3-cyano-7-fluorobenzo[b]thiophen-4- |
| | yl)-3,5-dimethyl-1H-pyrazol-1-yl)-2-((R)-1-((S)-1-methylpyrrolidin-2- |
| | yl)ethoxy)pyrimidine-5-carbonitrile |
| 656 | 4-(4-acetylpiperazin-1-yl)-6-((Rₐ)-4-(2-amino-3-cyano-7-fluorobenzo[b]thiophen-4- |
| | yl)-3,5-dimethyl-1H-pyrazol-1-yl)-2-((R)-1-((R)-1-methylpyrrolidin-2- |
| | yl)ethoxy)pyrimidine-5-carbonitrile |
| 657 | 4-(4-acetylpiperazin-1-yl)-6-((Sₐ)-4-(2-amino-3-cyano-7-fluorobenzo[b]thiophen-4- |
| | yl)-3,5-dimethyl-1H-pyrazol-1-yl)-2-((S)-1-((S)-1-methylpyrrolidin-2- |
| | yl)ethoxy)pyrimidine-5-carbonitrile |
| 658 | 4-(4-acetylpiperazin-1-yl)-6-((Sₐ)-4-(2-amino-3-cyano-7-fluorobenzo[b]thiophen-4- |
| | yl)-3,5-dimethyl-1H-pyrazol-1-yl)-2-((S)-1-((R)-1-methylpyrrolidin-2- |
| | yl)ethoxy)pyrimidine-5-carbonitrile |
| 659 | 4-(4-acetylpiperazin-1-yl)-6-((Sₐ)-4-(2-amino-3-cyano-7-fluorobenzo[b]thiophen-4- |
| | yl)-3,5-dimethyl-1H-pyrazol-1-yl)-2-((R)-1-((S)-1-methylpyrrolidin-2- |
| | yl)ethoxy)pyrimidine-5-carbonitrile |
| 660 | 4-(4-acetylpiperazin-1-yl)-6-((Sₐ)-4-(2-amino-3-cyano-7-fluorobenzo[b]thiophen-4- |
| | yl)-3,5-dimethyl-1H-pyrazol-1-yl)-2-((R)-1-((R)-1-methylpyrrolidin-2- |
| | yl)ethoxy)pyrimidine-5-carbonitrile |
| 661 | 2-amino-4-(1-(4-(4-(cyclopropanecarbonyl)piperazin-1-yl)-6-((S)-1-((S)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 662 | 2-amino-4-(1-(4-(4-(cyclopropanecarbonyl)piperazin-1-yl)-6-((S)-1-((R)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile; |
| 663 | 2-amino-4-(1-(4-(4-(cyclopropanecarbonyl)piperazin-1-yl)-6-((R)-1-((S)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 664 | 2-amino-4-(1-(4-(4-(cyclopropanecarbonyl)piperazin-1-yl)-6-((R)-1-((R)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 665 | 2-amino-(Rₐ)-4-(1-(4-(4-(cyclopropanecarbonyl)piperazin-1-yl)-6-((S)-1-((S)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 666 | 2-amino-(Rₐ)-4-(1-(4-(4-(cyclopropanecarbonyl)piperazin-1-yl)-6-((S)-1-((R)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile; |
| 667 | 2-amino-(Rₐ)-4-(1-(4-(4-(cyclopropanecarbonyl)piperazin-1-yl)-6-((R)-1-((S)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 668 | 2-amino-(Rₐ)-4-(1-(4-(4-(cyclopropanecarbonyl)piperazin-1-yl)-6-((R)-1-((R)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 669 | 2-amino-(Sₐ)-4-(1-(4-(4-(cyclopropanecarbonyl)piperazin-1-yl)-6-((S)-1-((S)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 670 | 2-amino-(Sₐ)-4-(1-(4-(4-(cyclopropanecarbonyl)piperazin-1-yl)-6-((S)-1-((R)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile; |
| 671 | 2-amino-(Sₐ)-4-(1-(4-(4-(cyclopropanecarbonyl)piperazin-1-yl)-6-((R)-1-((S)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 672 | 2-amino-(Sₐ)-4-(1-(4-(4-(cyclopropanecarbonyl)piperazin-1-yl)-6-((R)-1-((R)-1- |
| | methylpyrrolidin-2-yl)ethoxy)pyrimidin-2-yl)-3-methyl-1H-pyrazol-4-yl)-7- |
| | fluorobenzo[b]thiophene-3-carbonitrile |
| 673 | 2-amino-7-fluoro-4-(1-(2-(((2R,7aS)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 674 | 2-amino-7-fluoro-4-(1-(2-(((2S,7aR)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 675 | 2-amino-7-fluoro-4-(1-(2-(((2R,7aS)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 676 | 2-amino-7-fluoro-4-(1-(2-(((2R,7aR)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 677 | 2-amino-7-fluoro-(Rₐ)-4-(1-(2-(((2R,7aS)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 678 | 2-amino-7-fluoro-(Rₐ)-4-(1-(2-(((2S,7aR)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 679 | 2-amino-7-fluoro-(Rₐ)-4-(1-(2-(((2R,7aS)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 680 | 2-amino-7-fluoro-(Rₐ)-4-(1-(2-(((2R,7aR)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 681 | 2-amino-7-fluoro-(Sₐ)-4-(1-(2-(((2R,7aS)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 682 | 2-amino-7-fluoro-(Sₐ)-4-(1-(2-(((2S,7aR)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 683 | 2-amino-7-fluoro-(Sₐ)-4-(1-(2-(((2R,7aS)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 684 | 2-amino-7-fluoro-(Sₐ)-4-(1-(2-(((2R,7aR)-2-fluorotetrahydro-1H-pyrrolizin-7a(5H)- |
| | yl)methoxy)-6-(1-methyl-1H-pyrazol-4-yl)pyrimidin-4-yl)-3,5-dimethyl-1H-pyrazol- |
| | 4-yl)benzo[b]thiophene-3-carbonitrile |
| 685 | 1-(2-(4-(2-amino-3-cyano-7-fluorobenzo[b]thiophen-4-yl)-3,5-dimethyl-1H-pyrazol- |
| | 1-yl)-6-methylpyrimidin-4-yl)-1H-pyrazole-3-carboxamide |
| 686 | 1-(2-((Rₐ)-4-(2-amino-3-cyano-7-fluorobenzo[b]thiophen-4-yl)-3,5-dimethyl-1H- |
| | pyrazol-1-yl)-6-methylpyrimidin-4-yl)-1H-pyrazole-3-carboxamide |
| 687 | 1-(2-((Sₐ)-4-(2-amino-3-cyano-7-fluorobenzo[b]thiophen-4-yl)-3,5-dimethyl-1H- |
| | pyrazol-1-yl)-6-methylpyrimidin-4-yl)-1H-pyrazole-3-carboxamide |
| 688 | 2-amino-4-(1-(2-(4-(cyclopropanecarbonyl)piperazin-1-yl)-6-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 689 | 2-amino-4-(1-(2-(4-(cyclopropanecarbonyl)piperazin-1-yl)-6-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 690 | 2-amino-4-(1-(2-(4-(cyclopropanecarbonyl)piperazin-1-yl)-6-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 691 | 2-amino-4-(1-(2-(4-(cyclopropanecarbonyl)piperazin-1-yl)-6-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 692 | 2-amino-(Rₐ)-4-(1-(2-(4-(cyclopropanecarbonyl)piperazin-1-yl)-6-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 693 | 2-amino-(Rₐ)-4-(1-(2-(4-(cyclopropanecarbonyl)piperazin-1-yl)-6-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 694 | 2-amino-(Rₐ)-4-(1-(2-(4-(cyclopropanecarbonyl)piperazin-1-yl)-6-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 695 | 2-amino-(Rₐ)-4-(1-(2-(4-(cyclopropanecarbonyl)piperazin-1-yl)-6-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 696 | 2-amino-(Sₐ)-4-(1-(2-(4-(cyclopropanecarbonyl)piperazin-1-yl)-6-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 697 | 2-amino-(Sₐ)-4-(1-(2-(4-(cyclopropanecarbonyl)piperazin-1-yl)-6-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 698 | 2-amino-(Sₐ)-4-(1-(2-(4-(cyclopropanecarbonyl)piperazin-1-yl)-6-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 699 | 2-amino-(Sₐ)-4-(1-(2-(4-(cyclopropanecarbonyl)piperazin-1-yl)-6-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 57700 | 1-(2-(4-(2-amino-3-cyano-7-fluorobenzo[b]thiophen-4-yl)-3,5-dimethyl-1H-pyrazol- |
| | 1-yl)-6-((S)-1-((S)-1-methylpyrrolidin-2-yl)ethoxy)pyrimidin-4-yl)-N-(1- |
| | methylcyclopropyl)-1H-pyrazole-3-carboxamide |
| 701 | 1-(2-(4-(2-amino-3-cyano-7-fluorobenzo[b]thiophen-4-yl)-3,5-dimethyl-1H-pyrazol- |
| | 1-yl)-6-((S)-1-((R)-1-methylpyrrolidin-2-yl)ethoxy)pyrimidin-4-yl)-N-(1- |
| | methylcyclopropyl)-1H-pyrazole-3-carboxamide |
| 702 | 1-(2-(4-(2-amino-3-cyano-7-fluorobenzo[b]thiophen-4-yl)-3,5-dimethyl-1H-pyrazol- |
| | 1-yl)-6-((R)-1-((S)-1-methylpyrrolidin-2-yl)ethoxy)pyrimidin-4-yl)-N-(1- |
| | methylcyclopropyl)-1H-pyrazole-3-carboxamide |
| 703 | 1-(2-(4-(2-amino-3-cyano-7-fluorobenzo[b]thiophen-4-yl)-3,5-dimethyl-1H-pyrazol- |
| | 1-yl)-6-((R)-1-((R)-1-methylpyrrolidin-2-yl)ethoxy)pyrimidin-4-yl)-N-(1- |
| | methylcyclopropyl)-1H-pyrazole-3-carboxamide |
| 704 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N-(1- |
| | methylcyclopropyl)-1H-pyrazole-3-carboxamide |
| 705 | 1-(2-((Rₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N-(1- |
| | methylcyclopropyl)-1H-pyrazole-3-carboxamide |
| 706 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N-(1- |
| | methylcyclopropyl)-1H-pyrazole-3-carboxamide |
| 707 | 1-(2-((Rₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N-(1- |
| | methylcyclopropyl)-1H-pyrazole-3-carboxamide |
| 708 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((S)-1-methylpyrrolidin-2-yl)ethoxy)pyrimidin-4-yl)-N-(1- |
| | methylcyclopropyl)-1H-pyrazole-3-carboxamide |
| 709 | 1-(2-((Sₐ)-4-(2-amino-3-cyano-7-fluorobenzo[b]thiophen-4-yl)-3,5-dimethyl-1H- |
| | pyrazol-1-yl)-6-((S)-1-((R)-1-methylpyrrolidin-2-yl)ethoxy)pyrimidin-4-yl)-N-(1- |
| | methylcyclopropyl)-1H-pyrazole-3-carboxamide |
| 710 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((S)-1-methylpyrrolidin-2-yl)ethoxy)pyrimidin-4-yl)-N-(1- |
| | methylcyclopropyl)-1H-pyrazole-3-carboxamide |
| 711 | 1-(2-((Sₐ)-4-(2-amino-3-cyano-7-fluorobenzo[b]thiophen-4-yl)-3,5-dimethyl-1H- |
| | pyrazol-1-yl)-6-((R)-1-((R)-1-methylpyrrolidin-2-yl)ethoxy)pyrimidin-4-yl)-N-(1- |
| | methylcyclopropyl)-1H-pyrazole-3-carboxamide |
| 712 | 5-(6-(4-(2-amino-3-cyano-7-fluorobenzo[b]thiophen-4-yl)-3,5-dimethyl-1H-pyrazol- |
| | 1-yl)-2-((S)-1-((S)-1-methylpyrrolidin-2-yl)ethoxy)pyrimidin-4-yl)-N,N- |
| | dimethylpicolinamide |
| 713 | 5-(6-(4-(2-amino-3-cyano-7-fluorobenzo[b]thiophen-4-yl)-3,5-dimethyl-1H-pyrazol- |
| | 1-yl)-2-((S)-1-((R)-1-methylpyrrolidin-2-yl)ethoxy)pyrimidin-4-yl)-N,N- |
| | dimethylpicolinamide |
| 714 | 5-(6-(4-(2-amino-3-cyano-7-fluorobenzo[b]thiophen-4-yl)-3,5-dimethyl-1H-pyrazol- |
| | 1-yl)-2-((R)-1-((S)-1-methylpyrrolidin-2-yl)ethoxy)pyrimidin-4-yl)-N,N- |
| | dimethylpicolinamide |
| 715 | 5-(6-(4-(2-amino-3-cyano-7-fluorobenzo[b]thiophen-4-yl)-3,5-dimethyl-1H-pyrazol- |
| | 1-yl)-2-((R)-1-((R)-1-methylpyrrolidin-2-yl)ethoxy)pyrimidin-4-yl)-N,N- |
| | dimethylpicolinamide |
| 716 | 5-(6-((Rₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-N,N- |
| | dimethylpicolinamide |
| 717 | 5-(6-((Rₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-N,N- |
| | dimethylpicolinamide |
| 718 | 5-(6-((Rₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-N,N- |
| | dimethylpicolinamide |
| 719 | 5-(6-((Rₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-N,N- |
| | dimethylpicolinamide |
| 720 | 5-(6-((Sₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-N,N- |
| | dimethylpicolinamide |
| 721 | 5-(6-((Sₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-N,N- |
| | dimethylpicolinamide |
| 722 | 5-(6-((Sₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-N,N- |
| | dimethylpicolinamide |
| 723 | 5-(6-((Sₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-N,N- |
| | dimethylpicolinamide |
| 724 | 2-amino-4-(3,5-dimethyl-1-(6-(1-methyl-6-oxo-1,6-dihydropyridin-3-yl)-2-((S)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 725 | 2-amino-4-(3,5-dimethyl-1-(6-(1-methyl-6-oxo-1,6-dihydropyridin-3-yl)-2-((S)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 726 | 2-amino-4-(3,5-dimethyl-1-(6-(1-methyl-6-oxo-1,6-dihydropyridin-3-yl)-2-((R)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 727 | 2-amino-4-(3,5-dimethyl-1-(6-(1-methyl-6-oxo-1,6-dihydropyridin-3-yl)-2-((R)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 728 | 2-amino-(Rₐ)-4-(3,5-dimethyl-1-(6-(1-methyl-6-oxo-1,6-dihydropyridin-3-yl)-2-((S)- |
| | 1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 729 | 2-amino-(Rₐ)-4-(3,5-dimethyl-1-(6-(1-methyl-6-oxo-1,6-dihydropyridin-3-yl)-2-((S)- |
| | 1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 730 | 2-amino-(Rₐ)-4-(3,5-dimethyl-1-(6-(1-methyl-6-oxo-1,6-dihydropyridin-3-yl)-2-((R)- |
| | 1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 731 | 2-amino-(Rₐ)-4-(3,5-dimethyl-1-(6-(1-methyl-6-oxo-1,6-dihydropyridin-3-yl)-2-((R)- |
| | 1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 732 | 2-amino-(Sₐ)-4-(3,5-dimethyl-1-(6-(1-methyl-6-oxo-1,6-dihydropyridin-3-yl)-2-((S)- |
| | 1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 733 | 2-amino-(Sₐ)-4-(3,5-dimethyl-1-(6-(1-methyl-6-oxo-1,6-dihydropyridin-3-yl)-2-((S)- |
| | 1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 734 | 2-amino-(Sₐ)-4-(3,5-dimethyl-1-(6-(1-methyl-6-oxo-1,6-dihydropyridin-3-yl)-2-((R)- |
| | 1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 735 | 2-amino-(Sₐ)-4-(3,5-dimethyl-1-(6-(1-methyl-6-oxo-1,6-dihydropyridin-3-yl)-2-((R)- |
| | 1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 736 | 4-(6-(4-(2-amino-3-cyano-7-fluorobenzo[b]thiophen-4-yl)-3,5-dimethyl-1H-pyrazol- |
| | 1-yl)-2-((S)-1-((S)-1-methylpyrrolidin-2-yl)ethoxy)pyrimidin-4-yl)-2-fluoro-N- |
| | methylbenzamide |
| 737 | 4-(6-(4-(2-amino-3-cyano-7-fluorobenzo[b]thiophen-4-yl)-3,5-dimethyl-1H-pyrazol- |
| | 1-yl)-2-((S)-1-((R)-1-methylpyrrolidin-2-yl)ethoxy)pyrimidin-4-yl)-2-fluoro-N- |
| | methylbenzamide |
| 738 | 4-(6-(4-(2-amino-3-cyano-7-fluorobenzo[b]thiophen-4-yl)-3,5-dimethyl-1H-pyrazol- |
| | 1-yl)-2-((R)-1-((S)-1-methylpyrrolidin-2-yl)ethoxy)pyrimidin-4-yl)-2-fluoro-N- |
| | methylbenzamide |
| 739 | 4-(6-(4-(2-amino-3-cyano-7-fluorobenzo[b]thiophen-4-yl)-3,5-dimethyl-1H-pyrazol- |
| | 1-yl)-2-((R)-1-((R)-1-methylpyrrolidin-2-yl)ethoxy)pyrimidin-4-yl)-2-fluoro-N- |
| | methylbenzamide |
| 740 | 4-(6-((Rₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-2-fluoro- |
| | N-methylbenzamide |
| 741 | 4-(6-((Rₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-2-fluoro- |
| | N-methylbenzamide |
| 742 | 4-(6-((Rₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-2-fluoro- |
| | N-methylbenzamide |
| 743 | 4-(6-((Rₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-2-fluoro- |
| | N-methylbenzamide |
| 744 | 4-(6-((Sₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4-yl)-2-fluoro- |
| | N-methylbenzamide |
| 745 | 4-(6-((Sₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4-yl)-2-fluoro- |
| | N-methylbenzamide |
| 746 | 4-(6-((Sₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4-yl)-2-fluoro- |
| | N-methylbenzamide |
| 747 | 4-(6-((Sₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4-yl)-2-fluoro- |
| | N-methylbenzamide |
| 748 | 2-amino-4-(3,5-dimethyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 749 | 2-amino-4-(3,5-dimethyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 750 | 2-amino-4-(3,5-dimethyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 751 | 2-amino-4-(3,5-dimethyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 752 | 2-amino-(Rₐ)-4-(3,5-dimethyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 753 | 2-amino-(Rₐ)-4-(3,5-dimethyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 754 | 2-amino-(Rₐ)-4-(3,5-dimethyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 755 | 2-amino-(Rₐ)-4-(3,5-dimethyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 756 | 2-amino-(Sₐ)-4-(3,5-dimethyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 757 | 2-amino-(Sₐ)-4-(3,5-dimethyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 758 | 2-amino-(Sₐ)-4-(3,5-dimethyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 759 | 2-amino-(Sₐ)-4-(3,5-dimethyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 760 | 2-amino-4-(3,5-dimethyl-1-(6-(3-methyl-[1,2,4]triazolo[4,3-a]pyridin-6-yl)-2-((S)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 761 | 2-amino-4-(3,5-dimethyl-1-(6-(3-methyl-[1,2,4]triazolo[4,3-a]pyridin-6-yl)-2-((S)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile; |
| 762 | 2-amino-4-(3,5-dimethyl-1-(6-(3-methyl-[1,2,4]triazolo[4,3-a]pyridin-6-yl)-2-((R)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 763 | 2-amino-4-(3,5-dimethyl-1-(6-(3-methyl-[1,2,4]triazolo[4,3-a]pyridin-6-yl)-2-((R)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 764 | 2-amino-(Rₐ)-4-(3,5-dimethyl-1-(6-(3-methyl-[1,2,4]triazolo[4,3-a]pyridin-6-yl)-2- |
| | ((S)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 765 | 2-amino-(Rₐ)-4-(3,5-dimethyl-1-(6-(3-methyl-[1,2,4]triazolo[4,3-a]pyridin-6-yl)-2- |
| | ((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile; |
| 766 | 2-amino-(Rₐ)-4-(3,5-dimethyl-1-(6-(3-methyl-[1,2,4]triazolo[4,3-a]pyridin-6-yl)-2- |
| | ((R)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 767 | 2-amino-(Rₐ)-4-(3,5-dimethyl-1-(6-(3-methyl-[1,2,4]triazolo[4,3-a]pyridin-6-yl)-2- |
| | ((R)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 768 | 2-amino-(Sₐ)-4-(3,5-dimethyl-1-(6-(3-methyl-[1,2,4]triazolo[4,3-a]pyridin-6-yl)-2- |
| | ((S)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 769 | 2-amino-(Sₐ)-4-(3,5-dimethyl-1-(6-(3-methyl-[1,2,4]triazolo[4,3-a]pyridin-6-yl)-2- |
| | ((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile; |
| 770 | 2-amino-(Sₐ)-4-(3,5-dimethyl-1-(6-(3-methyl-[1,2,4]triazolo[4,3-a]pyridin-6-yl)-2- |
| | ((R)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 771 | 2-amino-(Sₐ)-4-(3,5-dimethyl-1-(6-(3-methyl-[1,2,4]triazolo[4,3-a]pyridin-6-yl)-2- |
| | ((R)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 772 | methyl (S)-2-(6-(4-(2-amino-3-cyano-7-fluorobenzo[b]thiophen-4-yl)-3,5-dimethyl- |
| | 1H-pyrazol-1-yl)-2-((S)-1-((S)-1-methylpyrrolidin-2-yl)ethoxy)pyrimidin-4- |
| | yl)pyrrolidine-1-carboxylate |
| 773 | methyl (S)-2-(6-(4-(2-amino-3-cyano-7-fluorobenzo[b]thiophen-4-yl)-3,5-dimethyl- |
| | 1H-pyrazol-1-yl)-2-((R)-1-((S)-1-methylpyrrolidin-2-yl)ethoxy)pyrimidin-4- |
| | yl)pyrrolidine-1-carboxylate |
| 774 | methyl (S)-2-(6-(4-(2-amino-3-cyano-7-fluorobenzo[b]thiophen-4-yl)-3,5-dimethyl- |
| | 1H-pyrazol-1-yl)-2-((S)-1-((R)-1-methylpyrrolidin-2-yl)ethoxy)pyrimidin-4- |
| | yl)pyrrolidine-1-carboxylate |
| 776 | methyl (S)-2-(6-(4-(2-amino-3-cyano-7-fluorobenzo[b]thiophen-4-yl)-3,5-dimethyl- |
| | 1H-pyrazol-1-yl)-2-((R)-1-((R)-1-methylpyrrolidin-2-yl)ethoxy)pyrimidin-4- |
| | yl)pyrrolidine-1-carboxylate |
| 778 | methyl (R)-2-(6-(4-(2-amino-3-cyano-7-fluorobenzo[b]thiophen-4-yl)-3,5-dimethyl- |
| | 1H-pyrazol-1-yl)-2-((S)-1-((S)-1-methylpyrrolidin-2-yl)ethoxy)pyrimidin-4- |
| | yl)pyrrolidine-1-carboxylate |
| 779 | methyl (R)-2-(6-(4-(2-amino-3-cyano-7-fluorobenzo[b]thiophen-4-yl)-3,5-dimethyl- |
| | 1H-pyrazol-1-yl)-2-((R)-1-((S)-1-methylpyrrolidin-2-yl)ethoxy)pyrimidin-4- |
| | yl)pyrrolidine-1-carboxylate |
| 780 | methyl (R)-2-(6-(4-(2-amino-3-cyano-7-fluorobenzo[b]thiophen-4-yl)-3,5-dimethyl- |
| | 1H-pyrazol-1-yl)-2-((S)-1-((R)-1-methylpyrrolidin-2-yl)ethoxy)pyrimidin-4- |
| | yl)pyrrolidine-1-carboxylate |
| 781 | methyl (R)-2-(6-(4-(2-amino-3-cyano-7-fluorobenzo[b]thiophen-4-yl)-3,5-dimethyl- |
| | 1H-pyrazol-1-yl)-2-((R)-1-((R)-1-methylpyrrolidin-2-yl)ethoxy)pyrimidin-4- |
| | yl)pyrrolidine-1-carboxylate |
| 782 | methyl (S)-2-(6-((Rₐ)-4-(2-amino-3-cyano-7-fluorobenzo[b]thiophen-4-yl)-3,5- |
| | dimethyl-1H-pyrazol-1-yl)-2-((S)-1-((S)-1-methylpyrrolidin-2-yl)ethoxy)pyrimidin-4- |
| | yl)pyrrolidine-1-carboxylate |
| 783 | methyl (S)-2-(6-((Rₐ)-4-(2-amino-3-cyano-7-fluorobenzo[b]thiophen-4-yl)-3,5- |
| | dimethyl-1H-pyrazol-1-yl)-2-((R)-1-((S)-1-methylpyrrolidin-2-yl)ethoxy)pyrimidin-4- |
| | yl)pyrrolidine-1-carboxylate |
| 784 | methyl (S)-2-(6-((Rₐ)-4-(2-amino-3-cyano-7-fluorobenzo[b]thiophen-4-yl)-3,5- |
| | dimethyl-1H-pyrazol-1-yl)-2-((S)-1-((R)-1-methylpyrrolidin-2-yl)ethoxy)pyrimidin-4- |
| | yl)pyrrolidine-1-carboxylate |
| 785 | methyl (S)-2-(6-((Rₐ)-4-(2-amino-3-cyano-7-fluorobenzo[b]thiophen-4-yl)-3,5- |
| | dimethyl-1H-pyrazol-1-yl)-2-((R)-1-((R)-1-methylpyrrolidin-2-yl)ethoxy)pyrimidin-4- |
| | yl)pyrrolidine-1-carboxylate |
| 786 | methyl (R)-2-(6-((Rₐ)-4-(2-amino-3-cyano-7-fluorobenzo[b]thiophen-4-yl)-3,5- |
| | dimethyl-1H-pyrazol-1-yl)-2-((S)-1-((S)-1-methylpyrrolidin-2-yl)ethoxy)pyrimidin-4- |
| | yl)pyrrolidine-1-carboxylate |
| 787 | methyl (R)-2-(6-((Rₐ)-4-(2-amino-3-cyano-7-fluorobenzo[b]thiophen-4-yl)-3,5- |
| | dimethyl-1H-pyrazol-1-yl)-2-((R)-1-((S)-1-methylpyrrolidin-2-yl)ethoxy)pyrimidin-4- |
| | yl)pyrrolidine-1-carboxylate |
| 788 | methyl (R)-2-(6-((Rₐ)-4-(2-amino-3-cyano-7-fluorobenzo[b]thiophen-4-yl)-3,5- |
| | dimethyl-1H-pyrazol-1-yl)-2-((S)-1-((R)-1-methylpyrrolidin-2-yl)ethoxy)pyrimidin-4- |
| | yl)pyrrolidine-1-carboxylate |
| 789 | methyl (R)-2-(6-((Rₐ)-4-(2-amino-3-cyano-7-fluorobenzo[b]thiophen-4-yl)-3,5- |
| | dimethyl-1H-pyrazol-1-yl)-2-((R)-1-((R)-1-methylpyrrolidin-2-yl)ethoxy)pyrimidin-4- |
| | yl)pyrrolidine-1-carboxylate |
| 790 | methyl (S)-2-(6-((Sₐ)-4-(2-amino-3-cyano-7-fluorobenzo[b]thiophen-4-yl)-3,5- |
| | dimethyl-1H-pyrazol-1-yl)-2-((S)-1-((S)-1-methylpyrrolidin-2-yl)ethoxy)pyrimidin-4- |
| | yl)pyrrolidine-1-carboxylate |
| 791 | methyl (S)-2-(6-((Sₐ)-4-(2-amino-3-cyano-7-fluorobenzo[b]thiophen-4-yl)-3,5- |
| | dimethyl-1H-pyrazol-1-yl)-2-((R)-1-((S)-1-methylpyrrolidin-2-yl)ethoxy)pyrimidin-4- |
| | yl)pyrrolidine-1-carboxylate |
| 792 | methyl (S)-2-(6-((Sₐ)-4-(2-amino-3-cyano-7-fluorobenzo[b]thiophen-4-yl)-3,5- |
| | dimethyl-1H-pyrazol-1-yl)-2-((S)-1-((R)-1-methylpyrrolidin-2-yl)ethoxy)pyrimidin-4- |
| | yl)pyrrolidine-1-carboxylate |
| 793 | methyl (S)-2-(6-((Sₐ)-4-(2-amino-3-cyano-7-fluorobenzo[b]thiophen-4-yl)-3,5- |
| | dimethyl-1H-pyrazol-1-yl)-2-((R)-1-((R)-1-methylpyrrolidin-2-yl)ethoxy)pyrimidin-4- |
| | yl)pyrrolidine-1-carboxylate |
| 794 | methyl (R)-2-(6-((Sₐ)-4-(2-amino-3-cyano-7-fluorobenzo[b]thiophen-4-yl)-3,5- |
| | dimethyl-1H-pyrazol-1-yl)-2-((S)-1-((S)-1-methylpyrrolidin-2-yl)ethoxy)pyrimidin-4- |
| | yl)pyrrolidine-1-carboxylate |
| 795 | methyl (R)-2-(6-((Sₐ)-4-(2-amino-3-cyano-7-fluorobenzo[b]thiophen-4-yl)-3,5- |
| | dimethyl-1H-pyrazol-1-yl)-2-((R)-1-((S)-1-methylpyrrolidin-2-yl)ethoxy)pyrimidin-4- |
| | yl)pyrrolidine-1-carboxylate |
| 796 | methyl (R)-2-(6-((Sₐ)-4-(2-amino-3-cyano-7-fluorobenzo[b]thiophen-4-yl)-3,5- |
| | dimethyl-1H-pyrazol-1-yl)-2-((S)-1-((R)-1-methylpyrrolidin-2-yl)ethoxy)pyrimidin-4- |
| | yl)pyrrolidine-1-carboxylate |
| 797 | methyl (R)-2-(6-((Sₐ)-4-(2-amino-3-cyano-7-fluorobenzo[b]thiophen-4-yl)-3,5- |
| | dimethyl-1H-pyrazol-1-yl)-2-((R)-1-((R)-1-methylpyrrolidin-2-yl)ethoxy)pyrimidin-4- |
| | yl)pyrrolidine-1-carboxylate |
| 798 | 2-amino-4-(3,5-dimethyl-1-(2-((S)-1-((S)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 799 | 2-amino-4-(3,5-dimethyl-1-(2-((S)-1-((R)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 800 | 2-amino-4-(3,5-dimethyl-1-(2-((R)-1-((S)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 801 | 2-amino-4-(3,5-dimethyl-1-(2-((R)-1-((R)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 802 | 2-amino-(Rₐ)-4-(3,5-dimethyl-1-(2-((S)-1-((S)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 803 | 2-amino-(Rₐ)-4-(3,5-dimethyl-1-(2-((S)-1-((R)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 804 | 2-amino-(Rₐ)-4-(3,5-dimethyl-1-(2-((R)-1-((S)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 805 | 2-amino-(Rₐ)-4-(3,5-dimethyl-1-(2-((R)-1-((R)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 806 | 2-amino-(Sₐ)-4-(3,5-dimethyl-1-(2-((S)-1-((S)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 807 | 2-amino-(Sₐ)-4-(3,5-dimethyl-1-(2-((S)-1-((R)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 808 | 2-amino-(Sₐ)-4-(3,5-dimethyl-1-(2-((R)-1-((S)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 809 | 2-amino-(Sₐ)-4-(3,5-dimethyl-1-(2-((R)-1-((R)-1-methylpyrrolidin-2-yl)ethoxy)-6-(6- |
| | oxooctahydro-2H-pyrido[1,2-a]pyrazin-2-yl)pyrimidin-4-yl)-1H-pyrazol-4-yl)-7- |
| | fluorobenzo[b]thiophene-3-carbonitrile |
| 810 | 2-amino-4-(3,5-dimethyl-1-(6-(6-methyl-5-oxo-2,6-diazaspiro[3.4]octan-2-yl)-2- |
| | ((S)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 811 | 2-amino-4-(3,5-dimethyl-1-(6-(6-methyl-5-oxo-2,6-diazaspiro[3.4]octan-2-yl)-2- |
| | ((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 812 | 2-amino-4-(3,5-dimethyl-1-(6-(6-methyl-5-oxo-2,6-diazaspiro[3.4]octan-2-yl)-2- |
| | ((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 813 | 2-amino-4-(3,5-dimethyl-1-(6-(6-methyl-5-oxo-2,6-diazaspiro[3.4]octan-2-yl)-2- |
| | ((R)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 814 | 2-amino-(Rₐ)-4-(3,5-dimethyl-1-(6-(6-methyl-5-oxo-2,6-diazaspiro[3.4]octan-2-yl)- |
| | 2-((S)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 815 | 2-amino-(Rₐ)-4-(3,5-dimethyl-1-(6-(6-methyl-5-oxo-2,6-diazaspiro[3.4]octan-2-yl)- |
| | 2-((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 816 | 2-amino-(Rₐ)-4-(3,5-dimethyl-1-(6-(6-methyl-5-oxo-2,6-diazaspiro[3.4]octan-2-yl)- |
| | 2-((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 817 | 2-amino-(Rₐ)-4-(3,5-dimethyl-1-(6-(6-methyl-5-oxo-2,6-diazaspiro[3.4]octan-2-yl)- |
| | 2-((R)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 818 | 2-amino-(Sₐ)-4-(3,5-dimethyl-1-(6-(6-methyl-5-oxo-2,6-diazaspiro[3.4]octan-2-yl)- |
| | 2-((S)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 819 | 2-amino-(Sₐ)-4-(3,5-dimethyl-1-(6-(6-methyl-5-oxo-2,6-diazaspiro[3.4]octan-2-yl)- |
| | 2-((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 820 | 2-amino-(Sₐ)-4-(3,5-dimethyl-1-(6-(6-methyl-5-oxo-2,6-diazaspiro[3.4]octan-2-yl)- |
| | 2-((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 821 | 2-amino-(Sₐ)-4-(3,5-dimethyl-1-(6-(6-methyl-5-oxo-2,6-diazaspiro[3.4]octan-2-yl)- |
| | 2-((R)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 822 | 4-(1-(6-((1S,4S)-2-oxa-5-azabicyclo[2.2.1]heptan-5-yl)-2-((S)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 823 | 4-(1-(6-((1S,4S)-2-oxa-5-azabicyclo[2.2.1]heptan-5-yl)-2-((S)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 824 | 4-(1-(6-((1S,4S)-2-oxa-5-azabicyclo[2.2.1]heptan-5-yl)-2-((R)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 825 | 4-(1-(6-((1S,4S)-2-oxa-5-azabicyclo[2.2.1]heptan-5-yl)-2-((R)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 826 | 4-(1-(6-((1R,4R)-2-oxa-5-azabicyclo[2.2.1]heptan-5-yl)-2-((S)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 827 | 4-(1-(6-((1R,4R)-2-oxa-5-azabicyclo[2.2.1]heptan-5-yl)-2-((R)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 828 | 4-(1-(6-((1R,4R)-2-oxa-5-azabicyclo[2.2.1]heptan-5-yl)-2-((S)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 829 | 4-(1-(6-((1R,4R)-2-oxa-5-azabicyclo[2.2.1]heptan-5-yl)-2-((R)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 830 | (Rₐ)-4-(1-(6-((1S,4S)-2-oxa-5-azabicyclo[2.2.1]heptan-5-yl)-2-((S)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 831 | (Rₐ)-4-(1-(6-((1S,4S)-2-oxa-5-azabicyclo[2.2.1]heptan-5-yl)-2-((S)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 832 | (Rₐ)-4-(1-(6-((1S,4S)-2-oxa-5-azabicyclo[2.2.1]heptan-5-yl)-2-((R)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 833 | (Rₐ)-4-(1-(6-((1S,4S)-2-oxa-5-azabicyclo[2.2.1]heptan-5-yl)-2-((R)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 834 | (Rₐ)-4-(1-(6-((1R,4R)-2-oxa-5-azabicyclo[2.2.1]heptan-5-yl)-2-((S)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 835 | (Rₐ)-4-(1-(6-((1R,4R)-2-oxa-5-azabicyclo[2.2.1]heptan-5-yl)-2-((R)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 836 | (Rₐ)-4-(1-(6-((1R,4R)-2-oxa-5-azabicyclo[2.2.1]heptan-5-yl)-2-((S)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 837 | (Rₐ)-4-(1-(6-((1R,4R)-2-oxa-5-azabicyclo[2.2.1]heptan-5-yl)-2-((R)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 838 | (Sₐ)-4-(1-(6-((1S,4S)-2-oxa-5-azabicyclo[2.2.1]heptan-5-yl)-2-((S)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 839 | (Sₐ)-4-(1-(6-((1S,4S)-2-oxa-5-azabicyclo[2.2.1]heptan-5-yl)-2-((S)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 840 | (Sₐ)-4-(1-(6-((1S,4S)-2-oxa-5-azabicyclo[2.2.1]heptan-5-yl)-2-((R)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 841 | (Sₐ)-4-(1-(6-((1S,4S)-2-oxa-5-azabicyclo[2.2.1]heptan-5-yl)-2-((R)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 842 | (Sₐ)-4-(1-(6-((1R,4R)-2-oxa-5-azabicyclo[2.2.1]heptan-5-yl)-2-((S)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 843 | (Sₐ)-4-(1-(6-((1R,4R)-2-oxa-5-azabicyclo[2.2.1]heptan-5-yl)-2-((R)-1-((S)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 844 | (Sₐ)-4-(1-(6-((1R,4R)-2-oxa-5-azabicyclo[2.2.1]heptan-5-yl)-2-((S)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 845 | (Sₐ)-4-(1-(6-((1R,4R)-2-oxa-5-azabicyclo[2.2.1]heptan-5-yl)-2-((R)-1-((R)-1- |
| | methylpyrrolidin-2-yl)ethoxy)pyrimidin-4-yl)-3,5-dimethyl-1H-pyrazol-4-yl)-2- |
| | amino-7-fluorobenzo[b]thiophene-3-carbonitrile |
| 846 | 2-amino-4-(3,5-dimethyl-1-(2-((S)-1-((S)-1-methylpyrrolidin-2-yl)ethoxy)-6-(pyridin- |
| | 4-yloxy)pyrimidin-4-yl)-1H-pyrazol-4-yl)-7-fluorobenzo[b]thiophene-3-carbonitrile |
| 847 | 2-amino-4-(3,5-dimethyl-1-(2-((S)-1-((R)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 848 | 2-amino-4-(3,5-dimethyl-1-(2-((R)-1-((S)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 849 | 2-amino-4-(3,5-dimethyl-1-(2-((R)-1-((R)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 850 | 2-amino-(Rₐ)-4-(3,5-dimethyl-1-(2-((S)-1-((S)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 851 | 2-amino-(Rₐ)-4-(3,5-dimethyl-1-(2-((S)-1-((R)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 852 | 2-amino-(Rₐ)-4-(3,5-dimethyl-1-(2-((R)-1-((S)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 853 | 2-amino-(Rₐ)-4-(3,5-dimethyl-1-(2-((R)-1-((R)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 854 | 2-amino-(Sₐ)-4-(3,5-dimethyl-1-(2-((S)-1-((S)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 855 | 2-amino-(Sₐ)-4-(3,5-dimethyl-1-(2-((S)-1-((R)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 856 | 2-amino-(Sₐ)-4-(3,5-dimethyl-1-(2-((R)-1-((S)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 857 | 2-amino-(Sₐ)-4-(3,5-dimethyl-1-(2-((R)-1-((R)-1-methylpyrrolidin-2-yl)ethoxy)-6- |
| | (pyridin-4-yloxy)pyrimidin-4-yl)-1H-pyrazol-4-yl)-7-fluorobenzo[b]thiophene-3- |
| | carbonitrile |
| 858 | 2-amino-7-fluoro-4-(1-(6-((3-fluoropyridin-4-yl)oxy)-2-((S)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 859 | 2-amino-7-fluoro-4-(1-(6-((3-fluoropyridin-4-yl)oxy)-2-((S)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 860 | 2-amino-7-fluoro-4-(1-(6-((3-fluoropyridin-4-yl)oxy)-2-((R)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 861 | 2-amino-7-fluoro-4-(1-(6-((3-fluoropyridin-4-yl)oxy)-2-((R)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 862 | 2-amino-7-fluoro-(Rₐ)-4-(1-(6-((3-fluoropyridin-4-yl)oxy)-2-((S)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 863 | 2-amino-7-fluoro-(Rₐ)-4-(1-(6-((3-fluoropyridin-4-yl)oxy)-2-((S)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 864 | 2-amino-7-fluoro-(Rₐ)-4-(1-(6-((3-fluoropyridin-4-yl)oxy)-2-((R)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 865 | 2-amino-7-fluoro-(Rₐ)-4-(1-(6-((3-fluoropyridin-4-yl)oxy)-2-((R)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 867 | 2-amino-7-fluoro-(Sₐ)-4-(1-(6-((3-fluoropyridin-4-yl)oxy)-2-((S)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 868 | 2-amino-7-fluoro-(Sₐ)-4-(1-(6-((3-fluoropyridin-4-yl)oxy)-2-((S)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 869 | 2-amino-7-fluoro-(Sₐ)-4-(1-(6-((3-fluoropyridin-4-yl)oxy)-2-((R)-1-((S)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 870 | 2-amino-7-fluoro-(Sₐ)-4-(1-(6-((3-fluoropyridin-4-yl)oxy)-2-((R)-1-((R)-1- |
| | methylpyrrolidin-2-yl)ethoxy)pyrimidin-4-yl)-3,5-dimethyl-1H-pyrazol-4- |
| | yl)benzo[b]thiophene-3-carbonitrile |
| 871 | 4-(1-(6-(1-acetylpiperidin-4-yl)-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 872 | 4-(1-(6-(1-acetylpiperidin-4-yl)-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 873 | 4-(1-(6-(1-acetylpiperidin-4-yl)-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 874 | 4-(1-(6-(1-acetylpiperidin-4-yl)-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 875 | (Rₐ)-4-(1-(6-(1-acetylpiperidin-4-yl)-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 876 | (Rₐ)-4-(1-(6-(1-acetylpiperidin-4-yl)-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 877 | (Rₐ)-4-(1-(6-(1-acetylpiperidin-4-yl)-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 878 | (Rₐ)-4-(1-(6-(1-acetylpiperidin-4-yl)-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 879 | (Sₐ)-4-(1-(6-(1-acetylpiperidin-4-yl)-2-((S)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 880 | (Sₐ)-4-(1-(6-(1-acetylpiperidin-4-yl)-2-((S)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 881 | (Sₐ)-4-(1-(6-(1-acetylpiperidin-4-yl)-2-((R)-1-((S)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 882 | (Sₐ)-4-(1-(6-(1-acetylpiperidin-4-yl)-2-((R)-1-((R)-1-methylpyrrolidin-2- |
| | yl)ethoxy)pyrimidin-4-yl)-3,5-dimethyl-1H-pyrazol-4-yl)-2-amino-7- |
| | fluorobenzo[b]thiophene-3-carbonitrile |
| 883 | 2-amino-4-(3,5-dimethyl-1-(4-(1-(methyl-d₃)-1H-pyrazol-4-yl)-6-((S)-1-((S)-1- |
| | methylpyrrolidin-2-yl)ethoxy)pyridin-2-yl)-1H-pyrazol-4-yl)-7- |
| | fluorobenzo[b]thiophene-3-carbonitrile |
| 884 | 2-amino-4-(3,5-dimethyl-1-(4-(1-(methyl-d₃)-1H-pyrazol-4-yl)-6-((S)-1-((R)-1- |
| | methylpyrrolidin-2-yl)ethoxy)pyridin-2-yl)-1H-pyrazol-4-yl)-7- |
| | fluorobenzo[b]thiophene-3-carbonitrile |
| 885 | 2-amino-4-(3,5-dimethyl-1-(4-(1-(methyl-d₃)-1H-pyrazol-4-yl)-6-((R)-1-((S)-1- |
| | methylpyrrolidin-2-yl)ethoxy)pyridin-2-yl)-1H-pyrazol-4-yl)-7- |
| | fluorobenzo[b]thiophene-3-carbonitrile |
| 886 | 2-amino-4-(3,5-dimethyl-1-(4-(1-(methyl-d₃)-1H-pyrazol-4-yl)-6-((R)-1-((R)-1- |
| | methylpyrrolidin-2-yl)ethoxy)pyridin-2-yl)-1H-pyrazol-4-yl)-7- |
| | fluorobenzo[b]thiophene-3-carbonitrile |
| 887 | 2-amino-(Rₐ)-4-(3,5-dimethyl-1-(4-(1-(methyl-d₃)-1H-pyrazol-4-yl)-6-((S)-1-((S)-1- |
| | methylpyrrolidin-2-yl)ethoxy)pyridin-2-yl)-1H-pyrazol-4-yl)-7- |
| | fluorobenzo[b]thiophene-3-carbonitrile |
| 888 | 2-amino-(Rₐ)-4-(3,5-dimethyl-1-(4-(1-(methyl-d₃)-1H-pyrazol-4-yl)-6-((S)-1-((R)-1- |
| | methylpyrrolidin-2-yl)ethoxy)pyridin-2-yl)-1H-pyrazol-4-yl)-7- |
| | fluorobenzo[b]thiophene-3-carbonitrile |
| 889 | 2-amino-(Rₐ)-4-(3,5-dimethyl-1-(4-(1-(methyl-d₃)-1H-pyrazol-4-yl)-6-((R)-1-((S)-1- |
| | methylpyrrolidin-2-yl)ethoxy)pyridin-2-yl)-1H-pyrazol-4-yl)-7- |
| | fluorobenzo[b]thiophene-3-carbonitrile |
| 890 | 2-amino-(Rₐ)-4-(3,5-dimethyl-1-(4-(1-(methyl-d₃)-1H-pyrazol-4-yl)-6-((R)-1-((R)-1- |
| | methylpyrrolidin-2-yl)ethoxy)pyridin-2-yl)-1H-pyrazol-4-yl)-7- |
| | fluorobenzo[b]thiophene-3-carbonitrile |
| 891 | 2-amino-(Sₐ)-4-(3,5-dimethyl-1-(4-(1-(methyl-d₃)-1H-pyrazol-4-yl)-6-((S)-1-((S)-1- |
| | methylpyrrolidin-2-yl)ethoxy)pyridin-2-yl)-1H-pyrazol-4-yl)-7- |
| | fluorobenzo[b]thiophene-3-carbonitrile |
| 892 | 2-amino-(Sₐ)-4-(3,5-dimethyl-1-(4-(1-(methyl-d₃)-1H-pyrazol-4-yl)-6-((S)-1-((R)-1- |
| | methylpyrrolidin-2-yl)ethoxy)pyridin-2-yl)-1H-pyrazol-4-yl)-7- |
| | fluorobenzo[b]thiophene-3-carbonitrile |
| 893 | 2-amino-(Sₐ)-4-(3,5-dimethyl-1-(4-(1-(methyl-d₃)-1H-pyrazol-4-yl)-6-((R)-1-((S)-1- |
| | methylpyrrolidin-2-yl)ethoxy)pyridin-2-yl)-1H-pyrazol-4-yl)-7- |
| | fluorobenzo[b]thiophene-3-carbonitrile |
| 894 | 2-amino-(Sₐ)-4-(3,5-dimethyl-1-(4-(1-(methyl-d₃)-1H-pyrazol-4-yl)-6-((R)-1-((R)-1- |
| | methylpyrrolidin-2-yl)ethoxy)pyridin-2-yl)-1H-pyrazol-4-yl)-7- |
| | fluorobenzo[b]thiophene-3-carbonitrile |
| 895 | (S)-4-(6-(4-(2-amino-3-cyano-7-fluorobenzo[b]thiophen-4-yl)-3,5-dimethyl-1H- |
| | pyrazol-1-yl)-2-((1-methylpyrrolidin-2-yl)methoxy)pyrimidin-4-yl)-N,N- |
| | dimethylpiperazine-1-carboxamide |
| 896 | (R)-4-(6-(4-(2-amino-3-cyano-7-fluorobenzo[b]thiophen-4-yl)-3,5-dimethyl-1H- |
| | pyrazol-1-yl)-2-((1-methylpyrrolidin-2-yl)methoxy)pyrimidin-4-yl)-N,N- |
| | dimethylpiperazine-1-carboxamide |
| 897 | (S)-4-(6-((Rₐ)-4-(2-amino-3-cyano-7-fluorobenzo[b]thiophen-4-yl)-3,5-dimethyl- |
| | 1H-pyrazol-1-yl)-2-((1-methylpyrrolidin-2-yl)methoxy)pyrimidin-4-yl)-N,N- |
| | dimethylpiperazine-1-carboxamide |
| 898 | (R)-4-(6-((Sₐ)-4-(2-amino-3-cyano-7-fluorobenzo[b]thiophen-4-yl)-3,5-dimethyl- |
| | 1H-pyrazol-1-yl)-2-((1-methylpyrrolidin-2-yl)methoxy)pyrimidin-4-yl)-N,N- |
| | dimethylpiperazine-1-carboxamide |
| 899 | (S)-4-(6-((Sₐ)-4-(2-amino-3-cyano-7-fluorobenzo[b]thiophen-4-yl)-3,5-dimethyl- |
| | 1H-pyrazol-1-yl)-2-((1-methylpyrrolidin-2-yl)methoxy)pyrimidin-4-yl)-N,N- |
| | dimethylpiperazine-1-carboxamide |
| 900 | (R)-4-(6-((Rₐ)-4-(2-amino-3-cyano-7-fluorobenzo[b]thiophen-4-yl)-3,5-dimethyl- |
| | 1H-pyrazol-1-yl)-2-((1-methylpyrrolidin-2-yl)methoxy)pyrimidin-4-yl)-N,N- |
| | dimethylpiperazine-1-carboxamide |
| 901 | (S)-4-(6-(4-(2-amino-3-cyano-7-fluorobenzo[b]thiophen-4-yl)-3,5-dimethyl-1H- |
| | pyrazol-1-yl)-2-((1-(dimethylamino)propan-2-yl)oxy)pyrimidin-4-yl)-N,N- |
| | dimethylpiperazine-1-carboxamide |
| 902 | (R)-4-(6-(4-(2-amino-3-cyano-7-fluorobenzo[b]thiophen-4-yl)-3,5-dimethyl-1H- |
| | pyrazol-1-yl)-2-((1-(dimethylamino)propan-2-yl)oxy)pyrimidin-4-yl)-N,N- |
| | dimethylpiperazine-1-carboxamide |
| 903 | (S)-4-(6-((Rₐ)-4-(2-amino-3-cyano-7-fluorobenzo[b]thiophen-4-yl)-3,5-dimethyl- |
| | 1H-pyrazol-1-yl)-2-((1-(dimethylamino)propan-2-yl)oxy)pyrimidin-4-yl)-N,N- |
| | dimethylpiperazine-1-carboxamide |
| 904 | (S)-4-(6-((Sₐ)-4-(2-amino-3-cyano-7-fluorobenzo[b]thiophen-4-yl)-3,5-dimethyl- |
| | 1H-pyrazol-1-yl)-2-((1-(dimethylamino)propan-2-yl)oxy)pyrimidin-4-yl)-N,N- |
| | dimethylpiperazine-1-carboxamide |
| 905 | (R)-4-(6-((Rₐ)-4-(2-amino-3-cyano-7-fluorobenzo[b]thiophen-4-yl)-3,5-dimethyl- |
| | 1H-pyrazol-1-yl)-2-((1-(dimethylamino)propan-2-yl)oxy)pyrimidin-4-yl)-N,N- |
| | dimethylpiperazine-1-carboxamide |
| 906 | (R)-4-(6-((Sₐ)-4-(2-amino-3-cyano-7-fluorobenzo[b]thiophen-4-yl)-3,5-dimethyl- |
| | 1H-pyrazol-1-yl)-2-((1-(dimethylamino)propan-2-yl)oxy)pyrimidin-4-yl)-N,N- |
| | dimethylpiperazine-1-carboxamide |
| 907 | 2-amino-4-(3,5-dimethyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7-fluorothieno[3,2- |
| | clpyridine-3-carbonitrile |
| 908 | 2-amino-4-(3,5-dimethyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7-fluorothieno[3,2- |
| | clpyridine-3-carbonitrile |
| 909 | 2-amino-4-(3,5-dimethyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 910 | 2-amino-4-(3,5-dimethyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 911 | 2-amino-(Rₐ)-4-(3,5-dimethyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 912 | 2-amino-(Rₐ)-4-(3,5-dimethyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 913 | 2-amino-(Rₐ)-4-(3,5-dimethyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 914 | 2-amino-(Rₐ)-4-(3,5-dimethyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 915 | 2-amino-(Sₐ)-4-(3,5-dimethyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 916 | 2-amino-(Sₐ)-4-(3,5-dimethyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7-fluorothieno[3,2- |
| | clpyridine-3-carbonitrile |
| 917 | 2-amino-(Sₐ)-4-(3,5-dimethyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 918 | 2-amino-(Sₐ)-4-(3,5-dimethyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 919 | 2-amino-4-(3-chloro-5-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 920 | 2-amino-4-(3-chloro-5-methyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 921 | 2-amino-4-(3-chloro-5-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 922 | 2-amino-4-(3-chloro-5-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 923 | 2-amino-(Rₐ)-4-(3-chloro-5-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 924 | 2-amino-(Rₐ)-4-(3-chloro-5-methyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 925 | 2-amino-(Rₐ)-4-(3-chloro-5-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 926 | 2-amino-(Rₐ)-4-(3-chloro-5-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 927 | 2-amino-(Sₐ)-4-(3-chloro-5-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 928 | 2-amino-(Sₐ)-4-(3-chloro-5-methyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 929 | 2-amino-(Sₐ)-4-(3-chloro-5-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 930 | 2-amino-(Sₐ)-4-(3-chloro-5-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 931 | 2-amino-7-fluoro-4-(3-(methoxymethyl)-5-methyl-1-(6-(1-methyl-1H-pyrazol-4-yl)- |
| | 2-((S)-1-((S)-1-methylpyrrolidin-2-yl)ethoxy)pyrimidin-4-yl)-1H-pyrazol-4- |
| | yl)benzo[b]thiophene-3-carbonitrile |
| 932 | 2-amino-7-fluoro-4-(3-(methoxymethyl)-5-methyl-1-(6-(1-methyl-1H-pyrazol-4-yl)- |
| | 2-((S)-1-((R)-1-methylpyrrolidin-2-yl)ethoxy)pyrimidin-4-yl)-1H-pyrazol-4- |
| | yl)benzo[b]thiophene-3-carbonitrile; |
| 933 | 2-amino-7-fluoro-4-(3-(methoxymethyl)-5-methyl-1-(6-(1-methyl-1H-pyrazol-4-yl)- |
| | 2-((R)-1-((S)-1-methylpyrrolidin-2-yl)ethoxy)pyrimidin-4-yl)-1H-pyrazol-4- |
| | yl)benzo[b]thiophene-3-carbonitrile |
| 934 | 2-amino-7-fluoro-4-(3-(methoxymethyl)-5-methyl-1-(6-(1-methyl-1H-pyrazol-4-yl)- |
| | 2-((R)-1-((R)-1-methylpyrrolidin-2-yl)ethoxy)pyrimidin-4-yl)-1H-pyrazol-4- |
| | yl)benzo[b]thiophene-3-carbonitrile |
| 935 | 2-amino-7-fluoro-(Rₐ)-4-(3-(methoxymethyl)-5-methyl-1-(6-(1-methyl-1H-pyrazol- |
| | 4-yl)-2-((S)-1-((S)-1-methylpyrrolidin-2-yl)ethoxy)pyrimidin-4-yl)-1H-pyrazol-4- |
| | yl)benzo[b]thiophene-3-carbonitrile |
| 936 | 2-amino-7-fluoro-(Rₐ)-4-(3-(methoxymethyl)-5-methyl-1-(6-(1-methyl-1H-pyrazol- |
| | 4-yl)-2-((S)-1-((R)-1-methylpyrrolidin-2-yl)ethoxy)pyrimidin-4-yl)-1H-pyrazol-4- |
| | yl)benzo[b]thiophene-3-carbonitrile; |
| 937 | 2-amino-7-fluoro-(Rₐ)-4-(3-(methoxymethyl)-5-methyl-1-(6-(1-methyl-1H-pyrazol- |
| | 4-yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 938 | 2-amino-7-fluoro-(Rₐ)-4-(3-(methoxymethyl)-5-methyl-1-(6-(1-methyl-1H-pyrazol- |
| | 4-yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 939 | 2-amino-7-fluoro-(Sₐ)-4-(3-(methoxymethyl)-5-methyl-1-(6-(1-methyl-1H-pyrazol- |
| | 4-yl)-2-((S)-1-((S)-1-methylpyrrolidin-2-yl)ethoxy)pyrimidin-4-yl)-1H-pyrazol-4- |
| | yl)benzo[b]thiophene-3-carbonitrile |
| 940 | 2-amino-7-fluoro-(Sₐ)-4-(3-(methoxymethyl)-5-methyl-1-(6-(1-methyl-1H-pyrazol- |
| | 4-yl)-2-((S)-1-((R)-1-methylpyrrolidin-2-yl)ethoxy)pyrimidin-4-yl)-1H-pyrazol-4- |
| | yl)benzo[b]thiophene-3-carbonitrile; |
| 941 | 2-amino-7-fluoro-(Sₐ)-4-(3-(methoxymethyl)-5-methyl-1-(6-(1-methyl-1H-pyrazol- |
| | 4-yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 941 | 2-amino-7-fluoro-(Sₐ)-4-(3-(methoxymethyl)-5-methyl-1-(6-(1-methyl-1H-pyrazol- |
| | 4-yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 942 | 2-amino-7-fluoro-4-(3-(methoxymethyl)-5-methyl-1-(6-(1-methyl-1H-pyrazol-4-yl)- |
| | 2-((S)-1-((S)-1-methylpyrrolidin-2-yl)ethoxy)pyrimidin-4-yl)-1H-pyrazol-4- |
| | yl)benzo[b]thiophene-3-carbonitrile |
| 943 | 2-amino-7-fluoro-4-(3-(methoxymethyl)-5-methyl-1-(6-(1-methyl-1H-pyrazol-4-yl)- |
| | 2-((S)-1-((R)-1-methylpyrrolidin-2-yl)ethoxy)pyrimidin-4-yl)-1H-pyrazol-4- |
| | yl)benzo[b]thiophene-3-carbonitrile |
| 944 | 2-amino-7-fluoro-4-(3-(methoxymethyl)-5-methyl-1-(6-(1-methyl-1H-pyrazol-4-yl)- |
| | 2-((R)-1-((S)-1-methylpyrrolidin-2-yl)ethoxy)pyrimidin-4-yl)-1H-pyrazol-4- |
| | yl)benzo[b]thiophene-3-carbonitrile |
| 945 | 2-amino-7-fluoro-4-(3-(methoxymethyl)-5-methyl-1-(6-(1-methyl-1H-pyrazol-4-yl)- |
| | 2-((R)-1-((R)-1-methylpyrrolidin-2-yl)ethoxy)pyrimidin-4-yl)-1H-pyrazol-4- |
| | yl)benzo[b]thiophene-3-carbonitrile |
| 946 | 2-amino-7-fluoro-(Rₐ)-4-(3-(methoxymethyl)-5-methyl-1-(6-(1-methyl-1H-pyrazol- |
| | 4-yl)-2-((S)-1-((S)-1-methylpyrrolidin-2-yl)ethoxy)pyrimidin-4-yl)-1H-pyrazol-4- |
| | yl)benzo[b]thiophene-3-carbonitrile |
| 947 | 2-amino-7-fluoro-(Rₐ)-4-(3-(methoxymethyl)-5-methyl-1-(6-(1-methyl-1H-pyrazol- |
| | 4-yl)-2-((S)-1-((R)-1-methylpyrrolidin-2-yl)ethoxy)pyrimidin-4-yl)-1H-pyrazol-4- |
| | yl)benzo[b]thiophene-3-carbonitrile |
| 948 | 2-amino-7-fluoro-(Rₐ)-4-(3-(methoxymethyl)-5-methyl-1-(6-(1-methyl-1H-pyrazol- |
| | 4-yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 949 | 2-amino-7-fluoro-(Rₐ)-4-(3-(methoxymethyl)-5-methyl-1-(6-(1-methyl-1H-pyrazol- |
| | 4-yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 950 | 2-amino-7-fluoro-(Sₐ)-4-(3-(methoxymethyl)-5-methyl-1-(6-(1-methyl-1H-pyrazol- |
| | 4-yl)-2-((S)-1-((S)-1-methylpyrrolidin-2-yl)ethoxy)pyrimidin-4-yl)-1H-pyrazol-4- |
| | yl)benzo[b]thiophene-3-carbonitrile |
| 951 | 2-amino-7-fluoro-(Sₐ)-4-(3-(methoxymethyl)-5-methyl-1-(6-(1-methyl-1H-pyrazol- |
| | 4-yl)-2-((S)-1-((R)-1-methylpyrrolidin-2-yl)ethoxy)pyrimidin-4-yl)-1H-pyrazol-4- |
| | yl)benzo[b]thiophene-3-carbonitrile |
| 952 | 2-amino-7-fluoro-(Sₐ)-4-(3-(methoxymethyl)-5-methyl-1-(6-(1-methyl-1H-pyrazol- |
| | 4-yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 953 | 2-amino-7-fluoro-(Sₐ)-4-(3-(methoxymethyl)-5-methyl-1-(6-(1-methyl-1H-pyrazol- |
| | 4-yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 954 | 2-amino-4-(3-cyclopropyl-5-methyl-1-(6-(1-methyl-1H-pyrazol-4-yl)-2-((S)-1-((S)- |
| | 1-methylpyrrolidin-2-yl)ethoxy)pyrimidin-4-yl)-1H-pyrazol-4-yl)-7- |
| | fluorobenzo[b]thiophene-3-carbonitrile |
| 955 | 2-amino-4-(3-cyclopropyl-5-methyl-1-(6-(1-methyl-1H-pyrazol-4-yl)-2-((S)-1-((R)- |
| | 1-methylpyrrolidin-2-yl)ethoxy)pyrimidin-4-yl)-1H-pyrazol-4-yl)-7- |
| | fluorobenzo[b]thiophene-3-carbonitrile |
| 956 | 2-amino-4-(3-cyclopropyl-5-methyl-1-(6-(1-methyl-1H-pyrazol-4-yl)-2-((R)-1-((S)- |
| | 1-methylpyrrolidin-2-yl)ethoxy)pyrimidin-4-yl)-1H-pyrazol-4-yl)-7- |
| | fluorobenzo[b]thiophene-3-carbonitrile |
| 957 | 2-amino-4-(3-cyclopropyl-5-methyl-1-(6-(1-methyl-1H-pyrazol-4-yl)-2-((R)-1-((R)- |
| | 1-methylpyrrolidin-2-yl)ethoxy)pyrimidin-4-yl)-1H-pyrazol-4-yl)-7- |
| | fluorobenzo[b]thiophene-3-carbonitrile |
| 958 | 2-amino-(Rₐ)-4-(3-cyclopropyl-5-methyl-1-(6-(1-methyl-1H-pyrazol-4-yl)-2-((S)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 959 | 2-amino-(Rₐ)-4-(3-cyclopropyl-5-methyl-1-(6-(1-methyl-1H-pyrazol-4-yl)-2-((S)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 960 | 2-amino-(Rₐ)-4-(3-cyclopropyl-5-methyl-1-(6-(1-methyl-1H-pyrazol-4-yl)-2-((R)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 961 | 2-amino-(Rₐ)-4-(3-cyclopropyl-5-methyl-1-(6-(1-methyl-1H-pyrazol-4-yl)-2-((R)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 962 | 2-amino-(Sₐ)-4-(3-cyclopropyl-5-methyl-1-(6-(1-methyl-1H-pyrazol-4-yl)-2-((S)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 963 | 2-amino-(Sₐ)-4-(3-cyclopropyl-5-methyl-1-(6-(1-methyl-1H-pyrazol-4-yl)-2-((S)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 964 | 2-amino-(Sₐ)-4-(3-cyclopropyl-5-methyl-1-(6-(1-methyl-1H-pyrazol-4-yl)-2-((R)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 965 | 2-amino-(Sₐ)-4-(3-cyclopropyl-5-methyl-1-(6-(1-methyl-1H-pyrazol-4-yl)-2-((R)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 966 | 2-amino-4-(3-(difluoromethyl)-5-methyl-1-(6-(1-methyl-1H-pyrazol-4-yl)-2-((S)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 967 | 2-amino-4-(3-(difluoromethyl)-5-methyl-1-(6-(1-methyl-1H-pyrazol-4-yl)-2-((S)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 968 | 2-amino-4-(3-(difluoromethyl)-5-methyl-1-(6-(1-methyl-1H-pyrazol-4-yl)-2-((R)-1- |
| | ((S)-1-methylpyrrolidin-2-yl)ethoxy)pyrimidin-4-yl)-1H-pyrazol-4-yl)-7- |
| | fluorobenzo[b]thiophene-3-carbonitrile |
| 969 | 2-amino-4-(3-(difluoromethyl)-5-methyl-1-(6-(1-methyl-1H-pyrazol-4-yl)-2-((R)-1- |
| | ((R)-1-methylpyrrolidin-2-yl)ethoxy)pyrimidin-4-yl)-1H-pyrazol-4-yl)-7- |
| | fluorobenzo[b]thiophene-3-carbonitrile |
| 970 | 2-amino-(Rₐ)-4-(3-(difluoromethyl)-5-methyl-1-(6-(1-methyl-1H-pyrazol-4-yl)-2- |
| | ((S)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 971 | 2-amino-(Rₐ)-4-(3-(difluoromethyl)-5-methyl-1-(6-(1-methyl-1H-pyrazol-4-yl)-2- |
| | ((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 972 | 2-amino-(Rₐ)-4-(3-(difluoromethyl)-5-methyl-1-(6-(1-methyl-1H-pyrazol-4-yl)-2- |
| | ((R)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 973 | 2-amino-(Rₐ)-4-(3-(difluoromethyl)-5-methyl-1-(6-(1-methyl-1H-pyrazol-4-yl)-2- |
| | ((R)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 974 | 2-amino-(Sₐ)-4-(3-(difluoromethyl)-5-methyl-1-(6-(1-methyl-1H-pyrazol-4-yl)-2- |
| | ((S)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 975 | 2-amino-(Sₐ)-4-(3-(difluoromethyl)-5-methyl-1-(6-(1-methyl-1H-pyrazol-4-yl)-2- |
| | ((S)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 976 | 2-amino-(Sₐ)-4-(3-(difluoromethyl)-5-methyl-1-(6-(1-methyl-1H-pyrazol-4-yl)-2- |
| | ((R)-1-((S)-1-methylpyrrolidin-2-yl)ethoxy)pyrimidin-4-yl)-1H-pyrazol-4-yl)-7- |
| | fluorobenzo[b]thiophene-3-carbonitrile |
| 977 | 2-amino-(Sₐ)-4-(3-(difluoromethyl)-5-methyl-1-(6-(1-methyl-1H-pyrazol-4-yl)-2- |
| | ((R)-1-((R)-1-methylpyrrolidin-2-yl)ethoxy)pyrimidin-4-yl)-1H-pyrazol-4-yl)-7- |
| | fluorobenzo[b]thiophene-3-carbonitrile |
| 978 | 2-amino-4-(5-ethyl-3-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 979 | 2-amino-4-(5-ethyl-3-methyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 980 | 2-amino-4-(5-ethyl-3-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 981 | 2-amino-4-(5-ethyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 982 | 2-amino-(Rₐ)-4-(5-ethyl-3-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 983 | 2-amino-(Rₐ)-4-(5-ethyl-3-methyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 984 | 2-amino-(Rₐ)-4-(5-ethyl-3-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 985 | 2-amino-(Rₐ)-4-(5-ethyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 986 | 2-amino-(Sₐ)-4-(5-ethyl-3-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 987 | 2-amino-(Sₐ)-4-(5-ethyl-3-methyl-1-(6-(1-methyl-1H-pyrazol-4-yl)-2-((S)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 988 | 2-amino-(Sₐ)-4-(5-ethyl-3-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 989 | 2-amino-(Sₐ)-4-(5-ethyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 990 | 2-amino-7-fluoro-4-(3-methyl-1-(6-(1-methyl-1H-pyrazol-4-yl)-2-((S)-1-((S)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 991 | 2-amino-7-fluoro-4-(3-methyl-1-(6-(1-methyl-1H-pyrazol-4-yl)-2-((S)-1-((R)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 992 | 2-amino-7-fluoro-4-(3-methyl-1-(6-(1-methyl-1H-pyrazol-4-yl)-2-((R)-1-((S)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 993 | 2-amino-7-fluoro-4-(3-methyl-1-(6-(1-methyl-1H-pyrazol-4-yl)-2-((R)-1-((R)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 994 | 2-amino-7-fluoro-(Rₐ)-4-(3-methyl-1-(6-(1-methyl-1H-pyrazol-4-yl)-2-((S)-1-((S)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 995 | 2-amino-7-fluoro-(Rₐ)-4-(3-methyl-1-(6-(1-methyl-1H-pyrazol-4-yl)-2-((S)-1-((R)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 996 | 2-amino-7-fluoro-(Rₐ)-4-(3-methyl-1-(6-(1-methyl-1H-pyrazol-4-yl)-2-((R)-1-((S)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 997 | 2-amino-7-fluoro-(Rₐ)-4-(3-methyl-1-(6-(1-methyl-1H-pyrazol-4-yl)-2-((R)-1-((R)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 998 | 2-amino-7-fluoro-(Sₐ)-4-(3-methyl-1-(6-(1-methyl-1H-pyrazol-4-yl)-2-((S)-1-((S)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 999 | 2-amino-7-fluoro-(Sₐ)-4-(3-methyl-1-(6-(1-methyl-1H-pyrazol-4-yl)-2-((S)-1-((R)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 1000 | 2-amino-7-fluoro-(Sₐ)-4-(3-methyl-1-(6-(1-methyl-1H-pyrazol-4-yl)-2-((R)-1-((S)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 1001 | 2-amino-7-fluoro-(Sₐ)-4-(3-methyl-1-(6-(1-methyl-1H-pyrazol-4-yl)-2-((R)-1-((R)-1- |
| | methylpyrrolidin-2-yl)ethoxy)pyrimidin-4-yl)-5-vinyl-1H-pyrazol-4- |
| | yl)benzo[b]thiophene-3-carbonitrile |
| 1002 | 2-amino-7-fluoro-4-(3-(hydroxymethyl)-5-methyl-1-(6-(1-methyl-1H-pyrazol-4-yl)- |
| | 2-((S)-1-((S)-1-methylpyrrolidin-2-yl)ethoxy)pyrimidin-4-yl)-1H-pyrazol-4- |
| | yl)benzo[b]thiophene-3-carbonitrile |
| 1003 | 2-amino-7-fluoro-4-(3-(hydroxymethyl)-5-methyl-1-(6-(1-methyl-1H-pyrazol-4-yl)- |
| | 2-((S)-1-((R)-1-methylpyrrolidin-2-yl)ethoxy)pyrimidin-4-yl)-1H-pyrazol-4- |
| | yl)benzo[b]thiophene-3-carbonitrile |
| 1004 | 2-amino-7-fluoro-4-(3-(hydroxymethyl)-5-methyl-1-(6-(1-methyl-1H-pyrazol-4-yl)- |
| | 2-((R)-1-((S)-1-methylpyrrolidin-2-yl)ethoxy)pyrimidin-4-yl)-1H-pyrazol-4- |
| | yl)benzo[b]thiophene-3-carbonitrile |
| 1005 | 2-amino-7-fluoro-4-(3-(hydroxymethyl)-5-methyl-1-(6-(1-methyl-1H-pyrazol-4-yl)- |
| | 2-((R)-1-((R)-1-methylpyrrolidin-2-yl)ethoxy)pyrimidin-4-yl)-1H-pyrazol-4- |
| | yl)benzo[b]thiophene-3-carbonitrile |
| 1006 | 2-amino-7-fluoro-(Rₐ)-4-(3-(hydroxymethyl)-5-methyl-1-(6-(1-methyl-1H-pyrazol-4- |
| | yl)-2-((S)-1-((S)-1-methylpyrrolidin-2-yl)ethoxy)pyrimidin-4-yl)-1H-pyrazol-4- |
| | yl)benzo[b]thiophene-3-carbonitrile |
| 1007 | 2-amino-7-fluoro-(Rₐ)-4-(3-(hydroxymethyl)-5-methyl-1-(6-(1-methyl-1H-pyrazol-4- |
| | yl)-2-((S)-1-((R)-1-methylpyrrolidin-2-yl)ethoxy)pyrimidin-4-yl)-1H-pyrazol-4- |
| | yl)benzo[b]thiophene-3-carbonitrile |
| 1008 | 2-amino-7-fluoro-(Rₐ)-4-(3-(hydroxymethyl)-5-methyl-1-(6-(1-methyl-1H-pyrazol-4- |
| | yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 1009 | 2-amino-7-fluoro-(Rₐ)-4-(3-(hydroxymethyl)-5-methyl-1-(6-(1-methyl-1H-pyrazol-4- |
| | yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 1010 | 2-amino-7-fluoro-(Sₐ)-4-(3-(hydroxymethyl)-5-methyl-1-(6-(1-methyl-1H-pyrazol-4- |
| | yl)-2-((S)-1-((S)-1-methylpyrrolidin-2-yl)ethoxy)pyrimidin-4-yl)-1H-pyrazol-4- |
| | yl)benzo[b]thiophene-3-carbonitrile |
| 1011 | 2-amino-7-fluoro-(Sₐ)-4-(3-(hydroxymethyl)-5-methyl-1-(6-(1-methyl-1H-pyrazol-4- |
| | yl)-2-((S)-1-((R)-1-methylpyrrolidin-2-yl)ethoxy)pyrimidin-4-yl)-1H-pyrazol-4- |
| | yl)benzo[b]thiophene-3-carbonitrile |
| 1012 | 2-amino-7-fluoro-(Sₐ)-4-(3-(hydroxymethyl)-5-methyl-1-(6-(1-methyl-1H-pyrazol-4- |
| | yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 1013 | 2-amino-7-fluoro-(Sₐ)-4-(3-(hydroxymethyl)-5-methyl-1-(6-(1-methyl-1H-pyrazol-4- |
| | yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 1014 | 2-amino-7-fluoro-4-(5-(hydroxymethyl)-3-methyl-1-(6-(1-methyl-1H-pyrazol-4-yl)- |
| | 2-((S)-1-((S)-1-methylpyrrolidin-2-yl)ethoxy)pyrimidin-4-yl)-1H-pyrazol-4- |
| | yl)benzo[b]thiophene-3-carbonitrile. |
| 1015 | 2-amino-7-fluoro-4-(5-(hydroxymethyl)-3-methyl-1-(6-(1-methyl-1H-pyrazol-4-yl)- |
| | 2-((S)-1-((R)-1-methylpyrrolidin-2-yl)ethoxy)pyrimidin-4-yl)-1H-pyrazol-4- |
| | yl)benzo[b]thiophene-3-carbonitrile |
| 1016 | 2-amino-7-fluoro-4-(5-(hydroxymethyl)-3-methyl-1-(6-(1-methyl-1H-pyrazol-4-yl)- |
| | 2-((R)-1-((S)-1-methylpyrrolidin-2-yl)ethoxy)pyrimidin-4-yl)-1H-pyrazol-4- |
| | yl)benzo[b]thiophene-3-carbonitrile |
| 1017 | 2-amino-7-fluoro-4-(5-(hydroxymethyl)-3-methyl-1-(6-(1-methyl-1H-pyrazol-4-yl)- |
| | 2-((R)-1-((R)-1-methylpyrrolidin-2-yl)ethoxy)pyrimidin-4-yl)-1H-pyrazol-4- |
| | yl)benzo[b]thiophene-3-carbonitrile |
| 1018 | 2-amino-7-fluoro-(Rₐ)-4-(5-(hydroxymethyl)-3-methyl-1-(6-(1-methyl-1H-pyrazol-4- |
| | yl)-2-((S)-1-((S)-1-methylpyrrolidin-2-yl)ethoxy)pyrimidin-4-yl)-1H-pyrazol-4- |
| | yl)benzo[b]thiophene-3-carbonitrile. |
| 1019 | 2-amino-7-fluoro-(Rₐ)-4-(5-(hydroxymethyl)-3-methyl-1-(6-(1-methyl-1H-pyrazol-4- |
| | yl)-2-((S)-1-((R)-1-methylpyrrolidin-2-yl)ethoxy)pyrimidin-4-yl)-1H-pyrazol-4- |
| | yl)benzo[b]thiophene-3-carbonitrile |
| 1020 | 2-amino-7-fluoro-(Rₐ)-4-(5-(hydroxymethyl)-3-methyl-1-(6-(1-methyl-1H-pyrazol-4- |
| | yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 1021 | 2-amino-7-fluoro-(Rₐ)-4-(5-(hydroxymethyl)-3-methyl-1-(6-(1-methyl-1H-pyrazol-4- |
| | yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 1022 | 2-amino-7-fluoro-(Sₐ)-4-(5-(hydroxymethyl)-3-methyl-1-(6-(1-methyl-1H-pyrazol-4- |
| | yl)-2-((S)-1-((S)-1-methylpyrrolidin-2-yl)ethoxy)pyrimidin-4-yl)-1H-pyrazol-4- |
| | yl)benzo[b]thiophene-3-carbonitrile. |
| 1023 | 2-amino-7-fluoro-(Sₐ)-4-(5-(hydroxymethyl)-3-methyl-1-(6-(1-methyl-1H-pyrazol-4- |
| | yl)-2-((S)-1-((R)-1-methylpyrrolidin-2-yl)ethoxy)pyrimidin-4-yl)-1H-pyrazol-4- |
| | yl)benzo[b]thiophene-3-carbonitrile |
| 1024 | 2-amino-7-fluoro-(Sₐ)-4-(5-(hydroxymethyl)-3-methyl-1-(6-(1-methyl-1H-pyrazol-4- |
| | yl)-2-((R)-1-((S)-1-methylpyrrolidin-2-yl)ethoxy)pyrimidin-4-yl)-1H-pyrazol-4- |
| | yl)benzo[b]thiophene-3-carbonitrile |
| 1025 | 2-amino-7-fluoro-(Sₐ)-4-(5-(hydroxymethyl)-3-methyl-1-(6-(1-methyl-1H-pyrazol-4- |
| | yl)-2-((R)-1-((R)-1-methylpyrrolidin-2-yl)ethoxy)pyrimidin-4-yl)-1H-pyrazol-4- |
| | yl)benzo[b]thiophene-3-carbonitrile |
| 1026 | 4-(6-((Rₐ)-4-(2-amino-3-cyano-7-fluorobenzo[b]thiophen-4-yl)-3,5-dimethyl-1H- |
| | pyrazol-1-yl)-2-((1-((dimethylamino)methyl)cyclopropyl)methoxy)pyrimidin-4-yl)- |
| | N,N-dimethylpiperazine-1-carboxamide |
| 1027 | 4-(6-((Sₐ)-4-(2-amino-3-cyano-7-fluorobenzo[b]thiophen-4-yl)-3,5-dimethyl-1H- |
| | pyrazol-1-yl)-2-((1-((dimethylamino)methyl)cyclopropyl)methoxy)pyrimidin-4-yl)- |
| | N,N-dimethylpiperazine-1-carboxamide |
| 1028 | (S)-4-(6-((Rₐ)-4-(2-amino-3-cyano-7-fluorobenzo[b]thiophen-4-yl)-3,5-dimethyl- |
| | 1H-pyrazol-1-yl)-2-(3-(dimethylamino)pyrrolidin-1-yl)pyrimidin-4-yl)-N,N- |
| | dimethylpiperazine-1-carboxamide |
| 1029 | (S)-4-(6-((Sₐ)-4-(2-amino-3-cyano-7-fluorobenzo[b]thiophen-4-yl)-3,5-dimethyl- |
| | 1H-pyrazol-1-yl)-2-(3-(dimethylamino)pyrrolidin-1-yl)pyrimidin-4-yl)-N,N- |
| | dimethylpiperazine-1-carboxamide |
| 1030 | (R)-4-(6-((Rₐ)-4-(2-amino-3-cyano-7-fluorobenzo[b]thiophen-4-yl)-3,5-dimethyl- |
| | 1H-pyrazol-1-yl)-2-(3-(dimethylamino)pyrrolidin-1-yl)pyrimidin-4-yl)-N,N- |
| | dimethylpiperazine-1-carboxamide |
| 1031 | (R)-4-(6-((Sₐ)-4-(2-amino-3-cyano-7-fluorobenzo[b]thiophen-4-yl)-3,5-dimethyl- |
| | 1H-pyrazol-1-yl)-2-(3-(dimethylamino)pyrrolidin-1-yl)pyrimidin-4-yl)-N,N- |
| | dimethylpiperazine-1-carboxamide |
| 1032 | 4-(2-(3-acetamidopyrrolidin-1-yl)-6-((Rₐ)-4-(2-amino-3-cyano-7- |
| | fluorobenzo[b]thiophen-4-yl)-3,5-dimethyl-1H-pyrazol-1-yl)pyrimidin-4-yl)-N,N- |
| | dimethylpiperazine-1-carboxamide |
| 1033 | 4-(2-(3-acetamidopyrrolidin-1-yl)-6-((Sₐ)-4-(2-amino-3-cyano-7- |
| | fluorobenzo[b]thiophen-4-yl)-3,5-dimethyl-1H-pyrazol-1-yl)pyrimidin-4-yl)-N,N- |
| | dimethylpiperazine-1-carboxamide |
| 1034 | 4-(6-(4-(2-amino-3-cyano-7-fluorobenzo[b]thiophen-4-yl)-3,5-dimethyl-1H-pyrazol- |
| | 1-yl)-2-((3aS,6aS)-1-methylhexahydropyrrolo[3,4-b]pyrrol-5(1H)-yl)pyrimidin-4-yl)- |
| | 2-fluoro-N-methylbenzamide |
| 1035 | 4-(6-(4-(2-amino-3-cyano-7-fluorobenzo[b]thiophen-4-yl)-3,5-dimethyl-1H-pyrazol- |
| | 1-yl)-2-((3aR,6aR)-1-methylhexahydropyrrolo[3,4-b]pyrrol-5(1H)-yl)pyrimidin-4-yl)- |
| | 2-fluoro-N-methylbenzamide |
| 1036 | 4-(6-((Rₐ)-4-(2-amino-3-cyano-7-fluorobenzo[b]thiophen-4-yl)-3,5-dimethyl-1H- |
| | pyrazol-1-yl)-2-((3aS,6aS)-1-methylhexahydropyrrolo[3,4-b]pyrrol-5(1H)- |
| | yl)pyrimidin-4-yl)-2-fluoro-N-methylbenzamide |
| 1037 | 4-(6-((Sₐ)-4-(2-amino-3-cyano-7-fluorobenzo[b]thiophen-4-yl)-3,5-dimethyl-1H- |
| | pyrazol-1-yl)-2-((3aS,6aS)-1-methylhexahydropyrrolo[3,4-b]pyrrol-5(1H)- |
| | yl)pyrimidin-4-yl)-2-fluoro-N-methylbenzamide |
| 1038 | 4-(6-((Rₐ)-4-(2-amino-3-cyano-7-fluorobenzo[b]thiophen-4-yl)-3,5-dimethyl-1H- |
| | pyrazol-1-yl)-2-((3aR,6aR)-1-methylhexahydropyrrolo[3,4-b]pyrrol-5(1H)- |
| | yl)pyrimidin-4-yl)-2-fluoro-N-methylbenzamide |
| 1039 | 4-(6-((Sₐ)-4-(2-amino-3-cyano-7-fluorobenzo[b]thiophen-4-yl)-3,5-dimethyl-1H- |
| | pyrazol-1-yl)-2-((3aR,6aR)-1-methylhexahydropyrrolo[3,4-b]pyrrol-5(1H)- |
| | yl)pyrimidin-4-yl)-2-fluoro-N-methylbenzamide |
| 1040 | 4-(6-(4-(2-amino-3-cyano-7-fluorobenzo[b]thiophen-4-yl)-3,5-dimethyl-1H-pyrazol- |
| | 1-yl)-2-((4aS,7aS)-1-methyloctahydro-6H-pyrrolo[3,4-b]pyridin-6-yl)pyrimidin-4-yl)- |
| | 2-fluoro-N-methylbenzamide |
| 1041 | 4-(6-(4-(2-amino-3-cyano-7-fluorobenzo[b]thiophen-4-yl)-3,5-dimethyl-1H-pyrazol- |
| | 1-yl)-2-((4aS,7aS)-1-methyloctahydro-6H-pyrrolo[3,4-b]pyridin-6-yl)pyrimidin-4-yl)- |
| | 2-fluoro-N-methylbenzamide |
| 1042 | 4-(6-((Rₐ)-4-(2-amino-3-cyano-7-fluorobenzo[b]thiophen-4-yl)-3,5-dimethyl-1H- |
| | pyrazol-1-yl)-2-((4aS,7aS)-1-methyloctahydro-6H-pyrrolo[3,4-b]pyridin-6- |
| | yl)pyrimidin-4-yl)-2-fluoro-N-methylbenzamide |
| 1043 | 4-(6-((Sₐ)-4-(2-amino-3-cyano-7-fluorobenzo[b]thiophen-4-yl)-3,5-dimethyl-1H- |
| | pyrazol-1-yl)-2-((4aS,7aS)-1-methyloctahydro-6H-pyrrolo[3,4-b]pyridin-6- |
| | yl)pyrimidin-4-yl)-2-fluoro-N-methylbenzamide |
| 1045 | 4-(6-((Rₐ)-4-(2-amino-3-cyano-7-fluorobenzo[b]thiophen-4-yl)-3,5-dimethyl-1H- |
| | pyrazol-1-yl)-2-((4aR,7aR)-1-methyloctahydro-6H-pyrrolo[3,4-b]pyridin-6- |
| | yl)pyrimidin-4-yl)-2-fluoro-N-methylbenzamide |
| 1046 | 4-(6-((Sₐ)-4-(2-amino-3-cyano-7-fluorobenzo[b]thiophen-4-yl)-3,5-dimethyl-1H- |
| | pyrazol-1-yl)-2-((4aR,7aR)-1-methyloctahydro-6H-pyrrolo[3,4-b]pyridin-6- |
| | yl)pyrimidin-4-yl)-2-fluoro-N-methylbenzamide |
| 1047 | 2-amino-4-(5-ethynyl-3-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 1048 | 2-amino-4-(5-ethynyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 1049 | 2-amino-4-(5-ethynyl-3-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 1050 | 2-amino-4-(5-ethynyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-7- |
| | fluorobenzo[b]thiophene-3-carbonitrile |
| 1051 | 2-amino-4-(5-ethynyl-3-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-(Rₐ)-7- |
| | fluorobenzo[b]thiophene-3-carbonitrile |
| 1052 | 2-amino-4-(5-ethynyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-(Rₐ)-7- |
| | fluorobenzo[b]thiophene-3-carbonitrile |
| 1053 | 2-amino-4-(5-ethynyl-3-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-(Rₐ)-7- |
| | fluorobenzo[b]thiophene-3-carbonitrile |
| 1054 | 2-amino-4-(5-ethynyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-(Rₐ)-7- |
| | fluorobenzo[b]thiophene-3-carbonitrile |
| 1055 | 2-amino-4-(5-ethynyl-3-methyl-1-(6-(1-methyl-1H-pyrazol-4-yl)-2-((S)-1-((S)-1- |
| | methylpyrrolidin-2-yl)ethoxy)pyrimidin-4-yl)-1H-pyrazol-4-yl)-(Sₐ)-7- |
| | fluorobenzo[b]thiophene-3-carbonitrile |
| 1056 | 2-amino-4-(5-ethynyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-(Sₐ)-7- |
| | fluorobenzo[b]thiophene-3-carbonitrile |
| 1057 | 2-amino-4-(5-ethynyl-3-methyl-1-(6-(1-methyl-1H-pyrazol-4-yl)-2-((R)-1-((S)-1- |
| | methylpyrrolidin-2-yl)ethoxy)pyrimidin-4-yl)-1H-pyrazol-4-yl)-(Sₐ)-7- |
| | fluorobenzo[b]thiophene-3-carbonitrile |
| 1058 | 2-amino-4-(5-ethynyl-3-methyl-1-(6-(1-methyl-1H-pyrazol-4-yl)-2-((R)-1-((R)-1- |
| | methylpyrrolidin-2-yl)ethoxy)pyrimidin-4-yl)-1H-pyrazol-4-yl)-(Sₐ)-7- |
| | fluorobenzo[b]thiophene-3-carbonitrile |
| 1059 | N-(1-(6-(4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4- |
| | yl)ethyl)cyclopropanecarboxamide |
| 1060 | N-(1-(6-(4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4- |
| | yl)ethyl)cyclopropanecarboxamide |
| 1061 | N-(1-(6-(4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4- |
| | yl)ethyl)cyclopropanecarboxamide |
| 1062 | N-(1-(6-(4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4- |
| | yl)ethyl)cyclopropanecarboxamide |
| 1063 | N-(1-(6-((Rₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4- |
| | yl)ethyl)cyclopropanecarboxamide |
| 1064 | N-(1-(6-((Rₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4- |
| | yl)ethyl)cyclopropanecarboxamide |
| 1065 | N-(1-(6-((Rₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4- |
| | yl)ethyl)cyclopropanecarboxamide |
| 1066 | N-(1-(6-((Rₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4- |
| | yl)ethyl)cyclopropanecarboxamide |
| 1067 | N-(1-(6-((Sₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)pyrimidin-4- |
| | yl)ethyl)cyclopropanecarboxamide |
| 1068 | N-(1-(6-((Sₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)pyrimidin-4- |
| | yl)ethyl)cyclopropanecarboxamide |
| 1069 | N-(1-(6-((Sₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)pyrimidin-4- |
| | yl)ethyl)cyclopropanecarboxamide |
| 1070 | N-(1-(6-((Sₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)pyrimidin-4- |
| | yl)ethyl)cyclopropanecarboxamide |
| 1071 | 2-amino-4-(1-(6-((1-(cyclopropanecarbonyl)azetidin-2-yl)methyl)-2-((S)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 1072 | 2-amino-4-(1-(6-((1-(cyclopropanecarbonyl)azetidin-2-yl)methyl)-2-((R)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 1073 | 2-amino-4-(1-(6-((1-(cyclopropanecarbonyl)azetidin-2-yl)methyl)-2-((S)-1-((R)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 1074 | 2-amino-4-(1-(6-((1-(cyclopropanecarbonyl)azetidin-2-yl)methyl)-2-((R)-1-((S)-1- |
| | methylpyrrolidin-2-yl)ethoxy)pyrimidin-4-yl)-3,5-dimethyl-1H-pyrazol-4-yl)-7- |
| | fluorobenzo[b]thiophene-3-carbonitrile |
| 1075 | 2-amino-4-(1-(6-((1-(cyclopropanecarbonyl)azetidin-2-yl)methyl)-2-((S)-1-((S)-1- |
| | methylpyrrolidin-2-yl)ethoxy)pyrimidin-4-yl)-3,5-dimethyl-1H-pyrazol-4-yl)-(Rₐ)-7- |
| | fluorobenzo[b]thiophene-3-carbonitrile |
| 1076 | 2-amino-4-(1-(6-((1-(cyclopropanecarbonyl)azetidin-2-yl)methyl)-2-((R)-1-((R)-1- |
| | methylpyrrolidin-2-yl)ethoxy)pyrimidin-4-yl)-3,5-dimethyl-1H-pyrazol-4-yl)-(Rₐ)-7- |
| | fluorobenzo[b]thiophene-3-carbonitrile |
| 1077 | 2-amino-4-(1-(6-((1-(cyclopropanecarbonyl)azetidin-2-yl)methyl)-2-((S)-1-((R)-1- |
| | methylpyrrolidin-2-yl)ethoxy)pyrimidin-4-yl)-3,5-dimethyl-1H-pyrazol-4-yl)-(Rₐ)-7- |
| | fluorobenzo[b]thiophene-3-carbonitrile |
| 1078 | 2-amino-4-(1-(6-((1-(cyclopropanecarbonyl)azetidin-2-yl)methyl)-2-((R)-1-((S)-1- |
| | methylpyrrolidin-2-yl)ethoxy)pyrimidin-4-yl)-3,5-dimethyl-1H-pyrazol-4-yl)-(Rₐ)-7- |
| | fluorobenzo[b]thiophene-3-carbonitrile |
| 1079 | 2-amino-4-(1-(6-((1-(cyclopropanecarbonyl)azetidin-2-yl)methyl)-2-((S)-1-((S)-1- |
| | methylpyrrolidin-2-yl)ethoxy)pyrimidin-4-yl)-3,5-dimethyl-1H-pyrazol-4-yl)-(Sₐ)-7- |
| | fluorobenzo[b]thiophene-3-carbonitrile |
| 1080 | 2-amino-4-(1-(6-((1-(cyclopropanecarbonyl)azetidin-2-yl)methyl)-2-((R)-1-((R)-1- |
| | methylpyrrolidin-2-yl)ethoxy)pyrimidin-4-yl)-3,5-dimethyl-1H-pyrazol-4-yl)-(Sₐ)-7- |
| | fluorobenzo[b]thiophene-3-carbonitrile |
| 1081 | 2-amino-4-(1-(6-((1-(cyclopropanecarbonyl)azetidin-2-yl)methyl)-2-((S)-1-((R)-1- |
| | methylpyrrolidin-2-yl)ethoxy)pyrimidin-4-yl)-3,5-dimethyl-1H-pyrazol-4-yl)-(Sₐ)-7- |
| | fluorobenzo[b]thiophene-3-carbonitrile |
| 1082 | 2-amino-4-(1-(6-((1-(cyclopropanecarbonyl)azetidin-2-yl)methyl)-2-((R)-1-((S)-1- |
| | methylpyrrolidin-2-yl)ethoxy)pyrimidin-4-yl)-3,5-dimethyl-1H-pyrazol-4-yl)-(Sₐ)-7- |
| | fluorobenzo[b]thiophene-3-carbonitrile |
| 1083 | 6-(4-(2-amino-3-cyano-7-fluorobenzo[b]thiophen-4-yl)-3,5-dimethyl-1H-pyrazol-1- |
| | yl)-2-((S)-1-((S)-1-methylpyrrolidin-2-yl)ethoxy)-N-(1-(pyrimidin-5- |
| | yl)ethyl)pyrimidine-4-carboxamide |
| 1084 | 6-(4-(2-amino-3-cyano-7-fluorobenzo[b]thiophen-4-yl)-3,5-dimethyl-1H-pyrazol-1- |
| | yl)-2-((R)-1-((R)-1-methylpyrrolidin-2-yl)ethoxy)-N-(1-(pyrimidin-5- |
| | yl)ethyl)pyrimidine-4-carboxamide |
| 1085 | 6-(4-(2-amino-3-cyano-7-fluorobenzo[b]thiophen-4-yl)-3,5-dimethyl-1H-pyrazol-1- |
| | yl)-2-((R)-1-((S)-1-methylpyrrolidin-2-yl)ethoxy)-N-(1-(pyrimidin-5- |
| | yl)ethyl)pyrimidine-4-carboxamide |
| 1086 | 6-(4-(2-amino-3-cyano-7-fluorobenzo[b]thiophen-4-yl)-3,5-dimethyl-1H-pyrazol-1- |
| | yl)-2-((S)-1-((R)-1-methylpyrrolidin-2-yl)ethoxy)-N-(1-(pyrimidin-5- |
| | yl)ethyl)pyrimidine-4-carboxamide |
| 1087 | 6-((Rₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)-N-(1-(pyrimidin-5- |
| | yl)ethyl)pyrimidine-4-carboxamide |
| 1088 | 6-((Rₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)-N-(1-(pyrimidin-5- |
| | yl)ethyl)pyrimidine-4-carboxamide |
| 1089 | 6-((Rₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)-N-(1-(pyrimidin-5- |
| | yl)ethyl)pyrimidine-4-carboxamide |
| 1090 | 6-((Rₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)-N-(1-(pyrimidin-5- |
| | yl)ethyl)pyrimidine-4-carboxamide |
| 1091 | 6-((Sₐ)-4-(2-amino-3-cyano-7-fluorobenzo[b]thiophen-4-yl)-3,5-dimethyl-1H- |
| | pyrazol-1-yl)-2-((S)-1-((S)-1-methylpyrrolidin-2-yl)ethoxy)-N-(1-(pyrimidin-5- |
| | yl)ethyl)pyrimidine-4-carboxamide |
| 1092 | 6-((Sₐ)-4-(2-amino-3-cyano-7-fluorobenzo[b]thiophen-4-yl)-3,5-dimethyl-1H- |
| | pyrazol-1-yl)-2-((R)-1-((R)-1-methylpyrrolidin-2-yl)ethoxy)-N-(1-(pyrimidin-5- |
| | yl)ethyl)pyrimidine-4-carboxamide |
| 1093 | 6-((Sₐ)-4-(2-amino-3-cyano-7-fluorobenzo[b]thiophen-4-yl)-3,5-dimethyl-1H- |
| | pyrazol-1-yl)-2-((R)-1-((S)-1-methylpyrrolidin-2-yl)ethoxy)-N-(1-(pyrimidin-5- |
| | yl)ethyl)pyrimidine-4-carboxamide |
| 1094 | 6-((Sₐ)-4-(2-amino-3-cyano-7-fluorobenzo[b]thiophen-4-yl)-3,5-dimethyl-1H- |
| | pyrazol-1-yl)-2-((S)-1-((R)-1-methylpyrrolidin-2-yl)ethoxy)-N-(1-(pyrimidin-5- |
| | yl)ethyl)pyrimidine-4-carboxamide |
| 1095 | 2-amino-4-(5-ethyl-3-methyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 1096 | 2-amino-4-(5-ethyl-3-methyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 1097 | 2-amino-4-(5-ethyl-3-methyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-7-fluorothieno[3,2- |
| | clpyridine-3-carbonitrile |
| 1098 | 2-amino-4-(5-ethyl-3-methyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 1099 | 2-amino-4-(5-ethyl-3-methyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1100 | 2-amino-4-(5-ethyl-3-methyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1101 | 2-amino-4-(5-ethyl-3-methyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1102 | 2-amino-4-(5-ethyl-3-methyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1103 | 2-amino-4-(5-ethyl-3-methyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1104 | 2-amino-4-(5-ethyl-3-methyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1105 | 2-amino-4-(5-ethyl-3-methyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1106 | 2-amino-4-(5-ethyl-3-methyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1107 | 2-amino-4-(5-ethyl-3-methyl-1-(6-(2-methyl-1-oxoisoindolin-5-yl)-2-((3aS,6aS)-1- |
| | methylhexahydropyrrolo[3,4-b]pyrrol-5(1H)-yl)pyrimidin-4-yl)-1H-pyrazol-4-yl)-7- |
| | fluorothieno[3,2-c]pyridine-3-carbonitrile |
| 1108 | 2-amino-4-(5-ethyl-3-methyl-1-(6-(2-methyl-1-oxoisoindolin-5-yl)-2-((3aR,6aR)-1- |
| | methylhexahydropyrrolo[3,4-b]pyrrol-5(1H)-yl)pyrimidin-4-yl)-1H-pyrazol-4-yl)-7- |
| | fluorothieno[3,2-c]pyridine-3-carbonitrile |
| 1109 | 2-amino-4-(5-ethyl-3-methyl-1-(6-(2-methyl-1-oxoisoindolin-5-yl)-2-((3aS,6aS)-1- |
| | methylhexahydropyrrolo[3,4-b]pyrrol-5(1H)-yl)pyrimidin-4-yl)-1H-pyrazol-4-yl)- |
| | (Rₐ)-7-fluorothieno[3,2-c]pyridine-3-carbonitrile |
| 1110 | 2-amino-4-(5-ethyl-3-methyl-1-(6-(2-methyl-1-oxoisoindolin-5-yl)-2-((3aR,6aR)-1- |
| | methylhexahydropyrrolo[3,4-b]pyrrol-5(1H)-yl)pyrimidin-4-yl)-1H-pyrazol-4-yl)-7- |
| | (Rₐ)-fluorothieno[3,2-c]pyridine-3-carbonitrile |
| 1111 | 2-amino-4-(5-ethyl-3-methyl-1-(6-(2-methyl-1-oxoisoindolin-5-yl)-2-((3aS,6aS)-1- |
| | methylhexahydropyrrolo[3,4-b]pyrrol-5(1H)-yl)pyrimidin-4-yl)-1H-pyrazol-4-yl)- |
| | (Sₐ)-7-fluorothieno[3,2-c]pyridine-3-carbonitrile |
| 1112 | 2-amino-4-(5-ethyl-3-methyl-1-(6-(2-methyl-1-oxoisoindolin-5-yl)-2-((3aR,6aR)-1- |
| | methylhexahydropyrrolo[3,4-b]pyrrol-5(1H)-yl)pyrimidin-4-yl)-1H-pyrazol-4-yl)-7- |
| | (Sₐ)-fluorothieno[3,2-c]pyridine-3-carbonitrile |
| 1113 | 5-(6-(4-(2-amino-3-cyano-7-fluorothieno[3,2-c]pyridin-4-yl)-5-ethyl-3-methyl-1H- |
| | pyrazol-1-yl)-2-((S)-1-((S)-1-methylpyrrolidin-2-yl)ethoxy)pyrimidin-4-yl)-N- |
| | methylpicolinamide |
| 1114 | 5-(6-(4-(2-amino-3-cyano-7-fluorothieno[3,2-c]pyridin-4-yl)-5-ethyl-3-methyl-1H- |
| | pyrazol-1-yl)-2-((R)-1-((R)-1-methylpyrrolidin-2-yl)ethoxy)pyrimidin-4-yl)-N- |
| | methylpicolinamide |
| 1115 | 5-(6-(4-(2-amino-3-cyano-7-fluorothieno[3,2-c]pyridin-4-yl)-5-ethyl-3-methyl-1H- |
| | pyrazol-1-yl)-2-((S)-1-((R)-1-methylpyrrolidin-2-yl)ethoxy)pyrimidin-4-yl)-N- |
| | methylpicolinamide |
| 1116 | 5-(6-(4-(2-amino-3-cyano-7-fluorothieno[3,2-c]pyridin-4-yl)-5-ethyl-3-methyl-1H- |
| | pyrazol-1-yl)-2-((R)-1-((S)-1-methylpyrrolidin-2-yl)ethoxy)pyrimidin-4-yl)-N- |
| | methylpicolinamide |
| 1117 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((S)-1-((S)-1-methylpyrrolidin-2-yl)ethoxy)pyrimidin-4-yl)-N- |
| | methylpicolinamide |
| 1118 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((R)-1-((R)-1-methylpyrrolidin-2-yl)ethoxy)pyrimidin-4-yl)-N- |
| | methylpicolinamide |
| 1119 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((S)-1-((R)-1-methylpyrrolidin-2-yl)ethoxy)pyrimidin-4-yl)-N- |
| | methylpicolinamide |
| 1120 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((R)-1-((S)-1-methylpyrrolidin-2-yl)ethoxy)pyrimidin-4-yl)-N- |
| | methylpicolinamide |
| 1121 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((S)-1-((S)-1-methylpyrrolidin-2-yl)ethoxy)pyrimidin-4-yl)-N- |
| | methylpicolinamide |
| 1122 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((R)-1-((R)-1-methylpyrrolidin-2-yl)ethoxy)pyrimidin-4-yl)-N- |
| | methylpicolinamide |
| 1123 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((S)-1-((R)-1-methylpyrrolidin-2-yl)ethoxy)pyrimidin-4-yl)-N- |
| | methylpicolinamide |
| 1124 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((R)-1-((S)-1-methylpyrrolidin-2-yl)ethoxy)pyrimidin-4-yl)-N- |
| | methylpicolinamide |
| 1125 | 4-(6-(4-(2-amino-3-cyano-7-fluorothieno[3,2-c]pyridin-4-yl)-5-ethyl-3-methyl-1H- |
| | pyrazol-1-yl)-2-((S)-1-((S)-1-methylpyrrolidin-2-yl)ethoxy)pyrimidin-4-yl)-2-fluoro- |
| | N-methylbenzamide |
| 1126 | 4-(6-(4-(2-amino-3-cyano-7-fluorothieno[3,2-c]pyridin-4-yl)-5-ethyl-3-methyl-1H- |
| | pyrazol-1-yl)-2-((R)-1-((R)-1-methylpyrrolidin-2-yl)ethoxy)pyrimidin-4-yl)-2-fluoro- |
| | N-methylbenzamide |
| 1127 | 4-(6-(4-(2-amino-3-cyano-7-fluorothieno[3,2-c]pyridin-4-yl)-5-ethyl-3-methyl-1H- |
| | pyrazol-1-yl)-2-((S)-1-((R)-1-methylpyrrolidin-2-yl)ethoxy)pyrimidin-4-yl)-2-fluoro- |
| | N-methylbenzamide |
| 1128 | 4-(6-(4-(2-amino-3-cyano-7-fluorothieno[3,2-c]pyridin-4-yl)-5-ethyl-3-methyl-1H- |
| | pyrazol-1-yl)-2-((R)-1-((S)-1-methylpyrrolidin-2-yl)ethoxy)pyrimidin-4-yl)-2-fluoro- |
| | N-methylbenzamide |
| 1129 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((S)-1-((S)-1-methylpyrrolidin-2-yl)ethoxy)pyrimidin-4-yl)-2- |
| | fluoro-N-methylbenzamide |
| 1130 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((R)-1-((R)-1-methylpyrrolidin-2-yl)ethoxy)pyrimidin-4-yl)-2- |
| | fluoro-N-methylbenzamide |
| 1131 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((S)-1-((R)-1-methylpyrrolidin-2-yl)ethoxy)pyrimidin-4-yl)-2- |
| | fluoro-N-methylbenzamide |
| 1132 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((R)-1-((S)-1-methylpyrrolidin-2-yl)ethoxy)pyrimidin-4-yl)-2- |
| | fluoro-N-methylbenzamide |
| 1133 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((S)-1-((S)-1-methylpyrrolidin-2-yl)ethoxy)pyrimidin-4-yl)-2- |
| | fluoro-N-methylbenzamide |
| 1134 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((R)-1-((R)-1-methylpyrrolidin-2-yl)ethoxy)pyrimidin-4-yl)-2- |
| | fluoro-N-methylbenzamide |
| 1135 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((S)-1-((R)-1-methylpyrrolidin-2-yl)ethoxy)pyrimidin-4-yl)-2- |
| | fluoro-N-methylbenzamide |
| 1136 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((R)-1-((S)-1-methylpyrrolidin-2-yl)ethoxy)pyrimidin-4-yl)-2- |
| | fluoro-N-methylbenzamide |
| 1137 | 4-(6-(4-(2-amino-3-cyano-7-fluorothieno[3,2-c]pyridin-4-yl)-5-ethyl-3-methyl-1H- |
| | pyrazol-1-yl)-2-((S)-1-((S)-1-methylpyrrolidin-2-yl)ethoxy)pyrimidin-4-yl)-N- |
| | cyclopropyl-2-fluorobenzamide |
| 1138 | 4-(6-(4-(2-amino-3-cyano-7-fluorothieno[3,2-c]pyridin-4-yl)-5-ethyl-3-methyl-1H- |
| | pyrazol-1-yl)-2-((R)-1-((R)-1-methylpyrrolidin-2-yl)ethoxy)pyrimidin-4-yl)-N- |
| | cyclopropyl-2-fluorobenzamide |
| 1139 | 4-(6-(4-(2-amino-3-cyano-7-fluorothieno[3,2-c]pyridin-4-yl)-5-ethyl-3-methyl-1H- |
| | pyrazol-1-yl)-2-((S)-1-((R)-1-methylpyrrolidin-2-yl)ethoxy)pyrimidin-4-yl)-N- |
| | cyclopropyl-2-fluorobenzamide |
| 1140 | 4-(6-(4-(2-amino-3-cyano-7-fluorothieno[3,2-c]pyridin-4-yl)-5-ethyl-3-methyl-1H- |
| | pyrazol-1-yl)-2-((R)-1-((S)-1-methylpyrrolidin-2-yl)ethoxy)pyrimidin-4-yl)-N- |
| | cyclopropyl-2-fluorobenzamide |
| 1141 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((S)-1-((S)-1-methylpyrrolidin-2-yl)ethoxy)pyrimidin-4-yl)-N- |
| | cyclopropyl-2-fluorobenzamide |
| 1142 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((R)-1-((R)-1-methylpyrrolidin-2-yl)ethoxy)pyrimidin-4-yl)-N- |
| | cyclopropyl-2-fluorobenzamide |
| 1143 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((S)-1-((R)-1-methylpyrrolidin-2-yl)ethoxy)pyrimidin-4-yl)-N- |
| | cyclopropyl-2-fluorobenzamide |
| 1144 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((R)-1-((S)-1-methylpyrrolidin-2-yl)ethoxy)pyrimidin-4-yl)-N- |
| | cyclopropyl-2-fluorobenzamide |
| 1145 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((S)-1-((S)-1-methylpyrrolidin-2-yl)ethoxy)pyrimidin-4-yl)-N- |
| | cyclopropyl-2-fluorobenzamide |
| 1146 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((R)-1-((R)-1-methylpyrrolidin-2-yl)ethoxy)pyrimidin-4-yl)-N- |
| | cyclopropyl-2-fluorobenzamide |
| 1147 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((S)-1-((R)-1-methylpyrrolidin-2-yl)ethoxy)pyrimidin-4-yl)-N- |
| | cyclopropyl-2-fluorobenzamide |
| 1148 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((R)-1-((S)-1-methylpyrrolidin-2-yl)ethoxy)pyrimidin-4-yl)-N- |
| | cyclopropyl-2-fluorobenzamide |
| 1149 | 5-(6-(4-(2-amino-3-cyano-7-fluorothieno[3,2-c]pyridin-4-yl)-5-ethyl-3-methyl-1H- |
| | pyrazol-1-yl)-2-((3aS,6aS)-1-methylhexahydropyrrolo[3,4-b]pyrrol-5(1H)- |
| | yl)pyrimidin-4-yl)-3-fluoro-N-methylpicolinamide |
| 1150 | 5-(6-(4-(2-amino-3-cyano-7-fluorothieno[3,2-c]pyridin-4-yl)-5-ethyl-3-methyl-1H- |
| | pyrazol-1-yl)-2-((3aR,6aR)-1-methylhexahydropyrrolo[3,4-b]pyrrol-5(1H)- |
| | yl)pyrimidin-4-yl)-3-fluoro-N-methylpicolinamide |
| 1151 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((3aS,6aS)-1-methylhexahydropyrrolo[3,4-b]pyrrol-5(1H)- |
| | yl)pyrimidin-4-yl)-3-fluoro-N-methylpicolinamide |
| 1152 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((3aR,6aR)-1-methylhexahydropyrrolo[3,4-b]pyrrol-5(1H)- |
| | yl)pyrimidin-4-yl)-3-fluoro-N-methylpicolinamide |
| 1153 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((3aS,6aS)-1-methylhexahydropyrrolo[3,4-b]pyrrol-5(1H)- |
| | yl)pyrimidin-4-yl)-3-fluoro-N-methylpicolinamide |
| 1154 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((3aR,6aR)-1-methylhexahydropyrrolo[3,4-b]pyrrol-5(1H)- |
| | yl)pyrimidin-4-yl)-3-fluoro-N-methylpicolinamide |
| 1155 | 4-(6-(4-(2-amino-3-cyano-7-fluorothieno[3,2-c]pyridin-4-yl)-5-ethyl-3-methyl-1H- |
| | pyrazol-1-yl)-2-((3aS,6aS)-1-methylhexahydropyrrolo[3,4-b]pyrrol-5(1H)- |
| | yl)pyrimidin-4-yl)-N-cyclopropyl-2-fluorobenzamide |
| 1156 | 4-(6-(4-(2-amino-3-cyano-7-fluorothieno[3,2-c]pyridin-4-yl)-5-ethyl-3-methyl-1H- |
| | pyrazol-1-yl)-2-((3aR,6aR)-1-methylhexahydropyrrolo[3,4-b]pyrrol-5(1H)- |
| | yl)pyrimidin-4-yl)-N-cyclopropyl-2-fluorobenzamide |
| 1157 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((3aS,6aS)-1-methylhexahydropyrrolo[3,4-b]pyrrol-5(1H)- |
| | yl)pyrimidin-4-yl)-N-cyclopropyl-2-fluorobenzamide |
| 1158 | 4-(6-((Rₐ)-4-(2-amino-3-cyano-7-fluorothieno[3,2-c]pyridin-4-yl)-5-ethyl-3-methyl- |
| | 1H-pyrazol-1-yl)-2-((3aR,6aR)-1-methylhexahydropyrrolo[3,4-b]pyrrol-5(1H)- |
| | yl)pyrimidin-4-yl)-N-cyclopropyl-2-fluorobenzamide |
| 1159 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((3aS,6aS)-1-methylhexahydropyrrolo[3,4-b]pyrrol-5(1H)- |
| | yl)pyrimidin-4-yl)-N-cyclopropyl-2-fluorobenzamide |
| 1160 | 4-(6-((Sₐ)-4-(2-amino-3-cyano-7-fluorothieno[3,2-c]pyridin-4-yl)-5-ethyl-3-methyl- |
| | 1H-pyrazol-1-yl)-2-((3aR,6aR)-1-methylhexahydropyrrolo[3,4-b]pyrrol-5(1H)- |
| | yl)pyrimidin-4-yl)-N-cyclopropyl-2-fluorobenzamide |
| 1161 | 5-(6-(4-(2-amino-3-cyano-7-fluorothieno[3,2-c]pyridin-4-yl)-5-ethyl-3-methyl-1H- |
| | pyrazol-1-yl)-2-((3aS,6aS)-1-methylhexahydropyrrolo[3,4-b]pyrrol-5(1H)- |
| | yl)pyrimidin-4-yl)-N-cyclopropyl-3-fluoropicolinamide |
| 1162 | 5-(6-(4-(2-amino-3-cyano-7-fluorothieno[3,2-c]pyridin-4-yl)-5-ethyl-3-methyl-1H- |
| | pyrazol-1-yl)-2-((3aR,6aR)-1-methylhexahydropyrrolo[3,4-b]pyrrol-5(1H)- |
| | yl)pyrimidin-4-yl)-N-cyclopropyl-3-fluoropicolinamide |
| 1163 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((3aS,6aS)-1-methylhexahydropyrrolo[3,4-b]pyrrol-5(1H)- |
| | yl)pyrimidin-4-yl)-N-cyclopropyl-3-fluoropicolinamide |
| 1164 | 5-(6-((Rₐ)-4-(2-amino-3-cyano-7-fluorothieno[3,2-c]pyridin-4-yl)-5-ethyl-3-methyl- |
| | 1H-pyrazol-1-yl)-2-((3aR,6aR)-1-methylhexahydropyrrolo[3,4-b]pyrrol-5(1H)- |
| | yl)pyrimidin-4-yl)-N-cyclopropyl-3-fluoropicolinamide |
| 1165 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((3aS,6aS)-1-methylhexahydropyrrolo[3,4-b]pyrrol-5(1H)- |
| | yl)pyrimidin-4-yl)-N-cyclopropyl-3-fluoropicolinamide |
| 1166 | 5-(6-((Sₐ)-4-(2-amino-3-cyano-7-fluorothieno[3,2-c]pyridin-4-yl)-5-ethyl-3-methyl- |
| | 1H-pyrazol-1-yl)-2-((3aR,6aR)-1-methylhexahydropyrrolo[3,4-b]pyrrol-5(1H)- |
| | yl)pyrimidin-4-yl)-N-cyclopropyl-3-fluoropicolinamide |
| 1167 | 2-amino-4-(5-ethyl-1-(6-(2-methyl-1-oxoisoindolin-5-yl)-2-((S)-1-((S)-1- |
| | methylpyrrolidin-2-yl)ethoxy)pyrimidin-4-yl)-3-(trifluoromethyl)-1H-pyrazol-4-yl)-7- |
| | fluorothieno[3,2-c]pyridine-3-carbonitrile |
| 1168 | 2-amino-4-(5-ethyl-1-(6-(2-methyl-1-oxoisoindolin-5-yl)-2-((S)-1-((R)-1- |
| | methylpyrrolidin-2-yl)ethoxy)pyrimidin-4-yl)-3-(trifluoromethyl)-1H-pyrazol-4-yl)-7- |
| | fluorothieno[3,2-c]pyridine-3-carbonitrile |
| 1169 | 2-amino-4-(5-ethyl-1-(6-(2-methyl-1-oxoisoindolin-5-yl)-2-((R)-1-((S)-1- |
| | methylpyrrolidin-2-yl)ethoxy)pyrimidin-4-yl)-3-(trifluoromethyl)-1H-pyrazol-4-yl)-7- |
| | fluorothieno[3,2-c]pyridine-3-carbonitrile |
| 1170 | 2-amino-4-(5-ethyl-1-(6-(2-methyl-1-oxoisoindolin-5-yl)-2-((R)-1-((R)-1- |
| | methylpyrrolidin-2-yl)ethoxy)pyrimidin-4-yl)-3-(trifluoromethyl)-1H-pyrazol-4-yl)-7- |
| | fluorothieno[3,2-c]pyridine-3-carbonitrile |
| 1171 | 2-amino-4-(5-ethyl-1-(6-(2-methyl-1-oxoisoindolin-5-yl)-2-((S)-1-((S)-1- |
| | methylpyrrolidin-2-yl)ethoxy)pyrimidin-4-yl)-3-(trifluoromethyl)-1H-pyrazol-4-yl)- |
| | (Rₐ)-7-fluorothieno[3,2-c]pyridine-3-carbonitrile |
| 1172 | 2-amino-4-(5-ethyl-1-(6-(2-methyl-1-oxoisoindolin-5-yl)-2-((S)-1-((S)-1- |
| | methylpyrrolidin-2-yl)ethoxy)pyrimidin-4-yl)-3-(trifluoromethyl)-1H-pyrazol-4-yl)- |
| | (Sₐ)-7-fluorothieno[3,2-c]pyridine-3-carbonitrile |
| 1173 | 2-amino-4-(5-ethyl-1-(6-(2-methyl-1-oxoisoindolin-5-yl)-2-((S)-1-((R)-1- |
| | methylpyrrolidin-2-yl)ethoxy)pyrimidin-4-yl)-3-(trifluoromethyl)-1H-pyrazol-4-yl)- |
| | (Rₐ)-7-fluorothieno[3,2-c]pyridine-3-carbonitrile |
| 1174 | 2-amino-4-(5-ethyl-1-(6-(2-methyl-1-oxoisoindolin-5-yl)-2-((S)-1-((R)-1- |
| | methylpyrrolidin-2-yl)ethoxy)pyrimidin-4-yl)-3-(trifluoromethyl)-1H-pyrazol-4-yl)- |
| | (Sₐ)-7-fluorothieno[3,2-c]pyridine-3-carbonitrile |
| 1175 | 2-amino-4-(5-ethyl-1-(6-(2-methyl-1-oxoisoindolin-5-yl)-2-((R)-1-((S)-1- |
| | methylpyrrolidin-2-yl)ethoxy)pyrimidin-4-yl)-3-(trifluoromethyl)-1H-pyrazol-4-yl)- |
| | (Rₐ)-7-fluorothieno[3,2-c]pyridine-3-carbonitrile |
| 1176 | 2-amino-4-(5-ethyl-1-(6-(2-methyl-1-oxoisoindolin-5-yl)-2-((R)-1-((S)-1- |
| | methylpyrrolidin-2-yl)ethoxy)pyrimidin-4-yl)-3-(trifluoromethyl)-1H-pyrazol-4-yl)- |
| | (Sₐ)-7-fluorothieno[3,2-c]pyridine-3-carbonitrile |
| 1177 | 2-amino-4-(5-ethyl-1-(6-(2-methyl-1-oxoisoindolin-5-yl)-2-((R)-1-((R)-1- |
| | methylpyrrolidin-2-yl)ethoxy)pyrimidin-4-yl)-3-(trifluoromethyl)-1H-pyrazol-4-yl)- |
| | (Rₐ)-7-fluorothieno[3,2-c]pyridine-3-carbonitrile |
| 1178 | 2-amino-4-(5-ethyl-1-(6-(2-methyl-1-oxoisoindolin-5-yl)-2-((R)-1-((R)-1- |
| | methylpyrrolidin-2-yl)ethoxy)pyrimidin-4-yl)-3-(trifluoromethyl)-1H-pyrazol-4-yl)- |
| | (Sₐ)-7-fluorothieno[3,2-c]pyridine-3-carbonitrile |
| 1179 | 2-amino-4-(3,5-diethyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-7-fluorothieno[3,2- |
| | clpyridine-3-carbonitrile |
| 1180 | 2-amino-4-(3,5-diethyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 1181 | 2-amino-4-(3,5-diethyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 1182 | 2-amino-4-(3,5-diethyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-7-fluorothieno[3,2- |
| | c]pyridine-3-carbonitrile |
| 1183 | 2-amino-4-(3,5-diethyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1184 | 2-amino-4-(3,5-diethyl-1-(6-(2-methyl-1-oxoisoindolin-5-yl)-2-((S)-1-((S)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1185 | 2-amino-4-(3,5-diethyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1186 | 2-amino-4-(3,5-diethyl-1-(6-(2-methyl-1-oxoisoindolin-5-yl)-2-((S)-1-((R)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1187 | 2-amino-4-(3,5-diethyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1188 | 2-amino-4-(3,5-diethyl-1-(6-(2-methyl-1-oxoisoindolin-5-yl)-2-((R)-1-((S)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1189 | 2-amino-4-(3,5-diethyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-(Rₐ)-7- |
| | fluorothieno[3,2-c]pyridine-3-carbonitrile |
| 1190 | 2-amino-4-(3,5-diethyl-1-(6-(2-methyl-1-oxoisoindolin-5-yl)-2-((R)-1-((R)-1- |
| | methylpyrrolidin-2-yl)ethoxy)pyrimidin-4-yl)-1H-pyrazol-4-yl)-(Sₐ)-7- |
| | fluorothieno[3,2-c]pyridine-3-carbonitrile |
| 1191 | (S)-2-amino-4-(5-ethyl-3-methyl-1-(6-(2-methyl-1-oxoisoindolin-5-yl)-2-((1- |
| | methylpyrrolidin-2-yl)methoxy)pyrimidin-4-yl)-1H-pyrazol-4-yl)-7-fluorothieno[3,2- |
| | c]pyridine-3-carbonitrile |
| 1192 | (R)-2-amino-4-(5-ethyl-3-methyl-1-(6-(2-methyl-1-oxoisoindolin-5-yl)-2-((1- |
| | methylpyrrolidin-2-yl)methoxy)pyrimidin-4-yl)-1H-pyrazol-4-yl)-7-fluorothieno[3,2- |
| | c]pyridine-3-carbonitrile |
| 1193 | (S)-2-amino-4-(5-ethyl-3-methyl-1-(6-(2-methyl-1-oxoisoindolin-5-yl)-2-((1- |
| | methylpyrrolidin-2-yl)methoxy)pyrimidin-4-yl)-1H-pyrazol-4-yl)-(Rₐ)-7- |
| | fluorothieno[3,2-c]pyridine-3-carbonitrile |
| 1194 | (S)-2-amino-4-(5-ethyl-3-methyl-1-(6-(2-methyl-1-oxoisoindolin-5-yl)-2-((1- |
| | methylpyrrolidin-2-yl)methoxy)pyrimidin-4-yl)-1H-pyrazol-4-yl)-(Sₐ)-7- |
| | fluorothieno[3,2-c]pyridine-3-carbonitrile |
| 1195 | (R)-2-amino-4-(5-ethyl-3-methyl-1-(6-(2-methyl-1-oxoisoindolin-5-yl)-2-((1- |
| | methylpyrrolidin-2-yl)methoxy)pyrimidin-4-yl)-1H-pyrazol-4-yl)-(Rₐ)-7- |
| | fluorothieno[3,2-c]pyridine-3-carbonitrile |
| 1196 | (R)-2-amino-4-(5-ethyl-3-methyl-1-(6-(2-methyl-1-oxoisoindolin-5-yl)-2-((1- |
| | methylpyrrolidin-2-yl)methoxy)pyrimidin-4-yl)-1H-pyrazol-4-yl)-(Sₐ)-7- |
| | fluorothieno[3,2-c]pyridine-3-carbonitrile |
| 1197 | (S)-2-amino-4-(1-(2-(3-(dimethylamino)pyrrolidin-1-yl)-6-(2-methyl-1- |
| | oxoisoindolin-5-yl)pyrimidin-4-yl)-5-ethyl-3-methyl-1H-pyrazol-4-yl)-7- |
| | fluorothieno[3,2-c]pyridine-3-carbonitrile |
| 1198 | (R)-2-amino-4-(1-(2-(3-(dimethylamino)pyrrolidin-1-yl)-6-(2-methyl-1- |
| | oxoisoindolin-5-yl)pyrimidin-4-yl)-5-ethyl-3-methyl-1H-pyrazol-4-yl)-7- |
| | fluorothieno[3,2-c]pyridine-3-carbonitrile |
| 1199 | (S)-2-amino-4-(1-(2-(3-(dimethylamino)pyrrolidin-1-yl)-6-(2-methyl-1- |
| | oxoisoindolin-5-yl)pyrimidin-4-yl)-5-ethyl-3-methyl-1H-pyrazol-4-yl)-(Rₐ)-7- |
| | fluorothieno[3,2-c]pyridine-3-carbonitrile |
| 1200 | (S)-2-amino-4-(1-(2-(3-(dimethylamino)pyrrolidin-1-yl)-6-(2-methyl-1- |
| | oxoisoindolin-5-yl)pyrimidin-4-yl)-5-ethyl-3-methyl-1H-pyrazol-4-yl)-(Sₐ)-7- |
| | fluorothieno[3,2-c]pyridine-3-carbonitrile |
| 1201 | (R)-2-amino-4-(1-(2-(3-(dimethylamino)pyrrolidin-1-yl)-6-(2-methyl-1- |
| | oxoisoindolin-5-yl)pyrimidin-4-yl)-5-ethyl-3-methyl-1H-pyrazol-4-yl)-(Rₐ)-7- |
| | fluorothieno[3,2-c]pyridine-3-carbonitrile |
| 1202 | (R)-2-amino-4-(1-(2-(3-(dimethylamino)pyrrolidin-1-yl)-6-(2-methyl-1- |
| | oxoisoindolin-5-yl)pyrimidin-4-yl)-5-ethyl-3-methyl-1H-pyrazol-4-yl)-(Sₐ)-7- |
| | fluorothieno[3,2-c]pyridine-3-carbonitrile |
| 1203 | 2-amino-4-(5-ethyl-3-methyl-1-(6-(2-methyl-1-oxoisoindolin-5-yl)-2-((1S,5S)-6- |
| | methyl-3,6-diazabicyclo[3.2.0]heptan-3-yl)pyrimidin-4-yl)-1H-pyrazol-4-yl)-7- |
| | fluorothieno[3,2-c]pyridine-3-carbonitrile |
| 1204 | 2-amino-4-(5-ethyl-3-methyl-1-(6-(2-methyl-1-oxoisoindolin-5-yl)-2-((1S,5R)-6- |
| | methyl-3,6-diazabicyclo[3.2.0]heptan-3-yl)pyrimidin-4-yl)-1H-pyrazol-4-yl)-7- |
| | fluorothieno[3,2-c]pyridine-3-carbonitrile |
| 1205 | 2-amino-4-(5-ethyl-3-methyl-1-(6-(2-methyl-1-oxoisoindolin-5-yl)-2-((1R,5S)-6- |
| | methyl-3,6-diazabicyclo[3.2.0]heptan-3-yl)pyrimidin-4-yl)-1H-pyrazol-4-yl)-7- |
| | fluorothieno[3,2-c]pyridine-3-carbonitrile |
| 1206 | 2-amino-4-(5-ethyl-3-methyl-1-(6-(2-methyl-1-oxoisoindolin-5-yl)-2-((1R,5R)-6- |
| | methyl-3,6-diazabicyclo[3.2.0]heptan-3-yl)pyrimidin-4-yl)-1H-pyrazol-4-yl)-7- |
| | fluorothieno[3,2-c]pyridine-3-carbonitrile |
| 1207 | 2-amino-4-(5-ethyl-3-methyl-1-(6-(2-methyl-1-oxoisoindolin-5-yl)-2-((1S,5S)-6- |
| | methyl-3,6-diazabicyclo[3.2.0]heptan-3-yl)pyrimidin-4-yl)-1H-pyrazol-4-yl)-(Rₐ)-7- |
| | fluorothieno[3,2-c]pyridine-3-carbonitrile |
| 1208 | 2-amino-4-(5-ethyl-3-methyl-1-(6-(2-methyl-1-oxoisoindolin-5-yl)-2-((1S,5R)-6- |
| | methyl-3,6-diazabicyclo[3.2.0]heptan-3-yl)pyrimidin-4-yl)-1H-pyrazol-4-yl)-(Rₐ)-7- |
| | fluorothieno[3,2-c]pyridine-3-carbonitrile |
| 1209 | 2-amino-4-(5-ethyl-3-methyl-1-(6-(2-methyl-1-oxoisoindolin-5-yl)-2-((1R,5S)-6- |
| | methyl-3,6-diazabicyclo[3.2.0]heptan-3-yl)pyrimidin-4-yl)-1H-pyrazol-4-yl)-(Rₐ)-7- |
| | fluorothieno[3,2-c]pyridine-3-carbonitrile |
| 1210 | 2-amino-4-(5-ethyl-3-methyl-1-(6-(2-methyl-1-oxoisoindolin-5-yl)-2-((1R,5R)-6- |
| | methyl-3,6-diazabicyclo[3.2.0]heptan-3-yl)pyrimidin-4-yl)-1H-pyrazol-4-yl)-(Rₐ)-7- |
| | fluorothieno[3,2-c]pyridine-3-carbonitrile |
| 1211 | 2-amino-4-(5-ethyl-3-methyl-1-(6-(2-methyl-1-oxoisoindolin-5-yl)-2-((1S,5S)-6- |
| | methyl-3,6-diazabicyclo[3.2.0]heptan-3-yl)pyrimidin-4-yl)-1H-pyrazol-4-yl)-(Sₐ)-7- |
| | fluorothieno[3,2-c]pyridine-3-carbonitrile |
| 1212 | 2-amino-4-(5-ethyl-3-methyl-1-(6-(2-methyl-1-oxoisoindolin-5-yl)-2-((1S,5R)-6- |
| | methyl-3,6-diazabicyclo[3.2.0]heptan-3-yl)pyrimidin-4-yl)-1H-pyrazol-4-yl)-(Sₐ)-7- |
| | fluorothieno[3,2-c]pyridine-3-carbonitrile |
| 1213 | 2-amino-4-(5-ethyl-3-methyl-1-(6-(2-methyl-1-oxoisoindolin-5-yl)-2-((1R,5S)-6- |
| | methyl-3,6-diazabicyclo[3.2.0]heptan-3-yl)pyrimidin-4-yl)-1H-pyrazol-4-yl)-(Sₐ)-7- |
| | fluorothieno[3,2-c]pyridine-3-carbonitrile |
| 1214 | 2-amino-4-(5-ethyl-3-methyl-1-(6-(2-methyl-1-oxoisoindolin-5-yl)-2-((1R,5R)-6- |
| | methyl-3,6-diazabicyclo[3.2.0]heptan-3-yl)pyrimidin-4-yl)-1H-pyrazol-4-yl)-(Sₐ)-7- |
| | fluorothieno[3,2-c]pyridine-3-carbonitrile |
| 1215 | 2-amino-4-(5-ethyl-3-methyl-1-(6-(2-methyl-1-oxoisoindolin-5-yl)-2-((4aS,7aS)-1- |
| | methyloctahydro-6H-pyrrolo[3,4-b]pyridin-6-yl)pyrimidin-4-yl)-1H-pyrazol-4-yl)-7- |
| | fluorothieno[3,2-c]pyridine-3-carbonitrile |
| 1216 | 2-amino-4-(5-ethyl-3-methyl-1-(6-(2-methyl-1-oxoisoindolin-5-yl)-2-((4aS,7aR)-1- |
| | methyloctahydro-6H-pyrrolo[3,4-b]pyridin-6-yl)pyrimidin-4-yl)-1H-pyrazol-4-yl)-7- |
| | fluorothieno[3,2-c]pyridine-3-carbonitrile |
| 1217 | 2-amino-4-(5-ethyl-3-methyl-1-(6-(2-methyl-1-oxoisoindolin-5-yl)-2-((4aR,7aS)-1- |
| | methyloctahydro-6H-pyrrolo[3,4-b]pyridin-6-yl)pyrimidin-4-yl)-1H-pyrazol-4-yl)-7- |
| | fluorothieno[3,2-c]pyridine-3-carbonitrile |
| 1218 | 2-amino-4-(5-ethyl-3-methyl-1-(6-(2-methyl-1-oxoisoindolin-5-yl)-2-((4aR,7aR)-1- |
| | methyloctahydro-6H-pyrrolo[3,4-b]pyridin-6-yl)pyrimidin-4-yl)-1H-pyrazol-4-yl)-7- |
| | fluorothieno[3,2-c]pyridine-3-carbonitrile |
| 1219 | 2-amino-4-(5-ethyl-3-methyl-1-(6-(2-methyl-1-oxoisoindolin-5-yl)-2-((4aS,7aS)-1- |
| | methyloctahydro-6H-pyrrolo[3,4-b]pyridin-6-yl)pyrimidin-4-yl)-1H-pyrazol-4-yl)- |
| | (Rₐ)-7-fluorothieno[3,2-c]pyridine-3-carbonitrile |
| 1220 | 2-amino-4-(5-ethyl-3-methyl-1-(6-(2-methyl-1-oxoisoindolin-5-yl)-2-((4aS,7aS)-1- |
| | methyloctahydro-6H-pyrrolo[3,4-b]pyridin-6-yl)pyrimidin-4-yl)-1H-pyrazol-4-yl)- |
| | (Sₐ)-7-fluorothieno[3,2-c]pyridine-3-carbonitrile |
| 1221 | 2-amino-4-(5-ethyl-3-methyl-1-(6-(2-methyl-1-oxoisoindolin-5-yl)-2-((4aS,7aR)-1- |
| | methyloctahydro-6H-pyrrolo[3,4-b]pyridin-6-yl)pyrimidin-4-yl)-1H-pyrazol-4-yl)- |
| | (Rₐ)-7-fluorothieno[3,2-c]pyridine-3-carbonitrile |
| 1222 | 2-amino-4-(5-ethyl-3-methyl-1-(6-(2-methyl-1-oxoisoindolin-5-yl)-2-((4aS,7aR)-1- |
| | methyloctahydro-6H-pyrrolo[3,4-b]pyridin-6-yl)pyrimidin-4-yl)-1H-pyrazol-4-yl)- |
| | (Sₐ)-7-fluorothieno[3,2-c]pyridine-3-carbonitrile |
| 1223 | 2-amino-4-(5-ethyl-3-methyl-1-(6-(2-methyl-1-oxoisoindolin-5-yl)-2-((4aR,7aS)-1- |
| | methyloctahydro-6H-pyrrolo[3,4-b]pyridin-6-yl)pyrimidin-4-yl)-1H-pyrazol-4-yl)- |
| | (Rₐ)-7-fluorothieno[3,2-c]pyridine-3-carbonitrile |
| 1224 | 2-amino-4-(5-ethyl-3-methyl-1-(6-(2-methyl-1-oxoisoindolin-5-yl)-2-((4aR,7aS)-1- |
| | methyloctahydro-6H-pyrrolo[3,4-b]pyridin-6-yl)pyrimidin-4-yl)-1H-pyrazol-4-yl)- |
| | (Sₐ)-7-fluorothieno[3,2-c]pyridine-3-carbonitrile |
| 1225 | 2-amino-4-(5-ethyl-3-methyl-1-(6-(2-methyl-1-oxoisoindolin-5-yl)-2-((4aR,7aR)-1- |
| | methyloctahydro-6H-pyrrolo[3,4-b]pyridin-6-yl)pyrimidin-4-yl)-1H-pyrazol-4-yl)- |
| | (Rₐ)-7-fluorothieno[3,2-c]pyridine-3-carbonitrile |
| 1226 | 2-amino-4-(5-ethyl-3-methyl-1-(6-(2-methyl-1-oxoisoindolin-5-yl)-2-((4aR,7aR)-1- |
| | methyloctahydro-6H-pyrrolo[3,4-b]pyridin-6-yl)pyrimidin-4-yl)-1H-pyrazol-4-yl)- |
| | (Sₐ)-7-fluorothieno[3,2-c]pyridine-3-carbonitrile |
| 1227 | 2-amino-4-(1-(2-((3aS,6aS)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | 7-fluorothieno[3,2-c]pyridine-3-carbonitrile |
| 1228 | 2-amino-4-(1-(2-((3aS,6aR)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | 7-fluorothieno[3,2-c]pyridine-3-carbonitrile |
| 1229 | 2-amino-4-(1-(2-((3aR,6aS)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | 7-fluorothieno[3,2-c]pyridine-3-carbonitrile |
| 1230 | 2-amino-4-(1-(2-((3aR,6aR)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | 7-fluorothieno[3,2-c]pyridine-3-carbonitrile |
| 1231 | 2-amino-4-(1-(2-((3aS,6aS)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Rₐ)-7-fluorothieno[3,2-c]pyridine-3-carbonitrile |
| 1232 | 2-amino-4-(1-(2-((3aS,6aR)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Rₐ)-7-fluorothieno[3,2-c]pyridine-3-carbonitrile |
| 1233 | 2-amino-4-(1-(2-((3aR,6aS)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Rₐ)-7-fluorothieno[3,2-c]pyridine-3-carbonitrile |
| 1234 | 2-amino-4-(1-(2-((3aR,6aR)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Rₐ)-7-fluorothieno[3,2-c]pyridine-3-carbonitrile |
| 1235 | 2-amino-4-(1-(2-((3aS,6aS)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Sₐ)-7-fluorothieno[3,2-c]pyridine-3-carbonitrile |
| 1236 | 2-amino-4-(1-(2-((3aS,6aR)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Sₐ)-7-fluorothieno[3,2-c]pyridine-3-carbonitrile |
| 1237 | 2-amino-4-(1-(2-((3aR,6aS)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Sₐ)-7-fluorothieno[3,2-c]pyridine-3-carbonitrile |
| 1238 | 2-amino-4-(1-(2-((3aR,6aR)-1-cyclopropylhexahydropyrrolo[3,4-b]pyrrol-5(1H)-yl)- |
| | 6-(2-methyl-1-oxoisoindolin-5-yl)pyrimidin-4-yl)-5-ethyl-3-methyl-1H-pyrazol-4-yl)- |
| | (Sₐ)-7-fluorothieno[3,2-c]pyridine-3-carbonitrile |

In other embodiments, the compound of Formula (I) is in the form of a pharmaceutically acceptable salt. In other embodiments, the compound of Formula (I) is in the form of a free base or free acid, or other than in the form of a salt.

In another aspect, provided herein is a pharmaceutical composition comprising a compound of Formula (I), or any of the embodiments thereof, or a pharmaceutically acceptable salt thereof, and a pharmaceutically acceptable carrier.

It is further appreciated that certain features of the present disclosure, which are, for clarity, described in the context of separate embodiments, can also be provided in combination in a single embodiment (while the embodiments are intended to be combined as if written in multiply dependent form). Conversely, various features of the present disclosure which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The hydrogen atom is formally removed and replaced by a substituent. A single divalent substituent, e.g., oxo, can replace two hydrogen atoms. The term “optionally substituted” means unsubstituted or substituted. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule.

The term “Cₙ₋ₘ” indicates a range which includes the endpoints, wherein n and m are integers and indicate the number of carbons present in a chemical moiety. The term is intended to include each and every member in the indicated range. Thus, Cₙ₋ₘ includes each member in the series Cₙ, Cₙ₊₁, . . . Cₘ₋₁, and Cₘ. Examples include C₁₋₄ (which includes C₁, C₂, C₃, and C₄), C₁₋₆ (which includes C₁, C₂, C₃, C₄, C₅, and C₆) and the like.

The term “alkyl” employed alone or in combination with other terms, refers to a saturated hydrocarbon group that may be straight-chained or branched. The term “Cₙ₋ₘ alkyl,” refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkenyl,” employed alone or in combination with other terms, refers to a straight-chain or branched hydrocarbon group corresponding to an alkyl group having one or more carbon-carbon double bonds. The term “Cₙ₋ₘ alkenyl” refers to an alkenyl group having n to m carbon atoms. An alkenyl group formally corresponds to an alkene with one C—H bond replaced by the point of attachment of the alkenyl group to the remainder of the compound. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

The term “alkynyl,” employed alone or in combination with other terms, refers to a straight-chain or branched hydrocarbon group corresponding to an alkyl group having one or more carbon-carbon triple bonds. The term “Cₙ₋ₘ alkynyl” refers to an alkynyl group having n to m carbon atoms. An alkynyl group formally corresponds to an alkyne with one C—H bond replaced by the point of attachment of the alkyl group to the remainder of the compound. In some embodiments, the alkynyl moiety contains 2 to 6 or 2 to 4 carbon atoms. Example alkynyl groups include, but are not limited to, ethynyl, propyn-1-yl, propyn-2-yl, and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms. The term “Cₙ₋ₘ dialkoxy” refers to a linking group of formula —O—(Cₙ₋ₘ alkyl)-O—, the alkyl group of which has n to m carbons. Example dialkoxy groups include —OCH₂CH₂O—and OCH₂CH₂CH₂O—. In some embodiments, the two O atoms of a Cₙ₋ₘ dialkoxy group may be attached to the same B atom to form a 5- or 6-membered heterocycloalkyl group.

The term “amino,” employed alone or in combination with other terms, refers to a group of formula —NH₂, wherein the hydrogen atoms may be substituted with a substituent described herein. For example, “alkylamino” can refer to —NH(alkyl) and —N(alkyl)₂.

The terms “halo” or “halogen,” used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” refers to an alkyl group in which one or more of the hydrogen atoms has been replaced by a halogen atom. The term “Cn-rn haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “perfluoroalkyl” refers to a haloalkyl group in which all hydrogen atoms have been replaced by fluorine atoms. Example perfluoroalkyl groups include CF₃, C₂F₅, and C₃F₇.

The term “haloalkoxy,” employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” or “oxy” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “carbonyl,” employed alone or in combination with other terms, refers to a —C(═O)— group, which also may be written as C(O).

The term “sulfonyl” refers to a —SO₂— group wherein a sulfur atom is doubly bound to two oxygen atoms.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, isoindolyl, benzothiophenyl, benzofuranyl, benzisoxazolyl, imidazo[1,2-b]thiazolyl, purinyl, and the like. In some embodiments, the heteroaryl group is pyridone (e.g., 2-pyridone).

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl” or “cycloalkane” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” or “Cₙ₋ₘ cycloalkane” refers to a cycloalkyl or cycloalkane that has n to m ring member carbon atoms. Cycloalkyl groups can include mono- or polycyclic (e.g., having 2, 3 or 4 fused rings) groups and spirocycles. In the following, references to cycloalkyl groups apply equally to cycloalkane groups. Cycloalkyl groups can have 3, 4, 5, 6 or 7 ring-forming carbons (C₃-7). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl. In some embodiments, the cycloalkyl group is tetrahydronaphthalenyl (e.g., 1,2,3,4-tetrahydronaphthalenyl).

The term “heterocycloalkyl” or “heterocycloalkane” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. The term “n-m-membered heterocycloalkyl” or “n-m-membered heterocycloalkane” where n and m are integers refer to a heterocycloalkyl or heterocyclalkane ring or ring system containing from n to m ring-forming atoms. An n-m-membered heterocycloalkyl or heterocycloalkane includes from 1 to m-1 carbon atoms and from 1 to m-1 heteroatoms. The term “n-membered heterocycloalkyl” or “n-membered heterocycloalkane” where n is an integer refers to a heterocycloalkyl ring or ring system containing from n to m ring-forming atoms. In the following, references to heterocycloalkyl groups, rings or ring systems apply equally to heterocycloalkane groups, rings or ring systems. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 2,5-diazobicyclo[2.2.1]heptanyl; pyrrolidinyl; hexahydropyrrolo[3,4-b]pyrrol-1(2H)-yl; 1,6-dihydropyridinyl; morpholinyl; azetidinyl; piperazinyl; and 4,7-diazaspiro[2.5]octan-7-yl.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

When the compounds described herein contain a chiral center, unless otherwise indicated, the compounds can be any of the possible stereoisomers. In some embodiments, the compounds provided herein have the (R)-configuration. In other embodiments, the compounds have the (S)-configuration. In compounds with more than one chiral centers, each of the chiral centers in the compound may be independently (R) or (S), unless otherwise indicated. In compounds with a single chiral center, the stereochemistry of the chiral center can be (R) or (S). In compounds with two chiral centers, the stereochemistry of the chiral centers can each be independently (R) or (S) so the configuration of the chiral centers can be (R) and (R), (R) and (S); (S) and (R), or (S) and (S). In compounds with three chiral centers, the stereochemistry each of the three chiral centers can each be independently (R) or (S) so the configuration of the chiral centers can be (R), (R) and (R); (R), (R) and (S); (R), (S) and (R); (R), (S) and (S); (S), (R) and (R); (S), (R) and (S); (S), (S) and (R); or (S), (S) and (S).

Compounds of the present disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H—, 2H— and 4H-1,2,4-triazole, 1H- and 2H-isoindole and 1H- and 2H-pyrazole. Tautomeric forms can be in equilibrium or sterically locked into one form by appropriate substitution. Compounds herein identified by name or structure as one particular tautomeric form are intended to include other tautomeric forms unless otherwise specified (e.g., in the case of purine rings, unless otherwise indicated, if a compound name or structure described the 9H tautomer, it would be understood that the 7H tautomer is also encompassed).

Compounds provided herein can exist in the form of atropisomers (i.e., conformational diastereoisomers) that can be stable at ambient temperature and separable, e.g., by chromatography. For example, the compounds of Formula (I) can exist in the form of atropisomers that are interchangeable by rotation around the bond connecting Cy¹ (or any of the embodiments thereof) to the remainder of the molecule. Reference to the compounds described herein or any of the embodiments is understood to include all such atropisomeric forms of the compounds. Without being limited by any theory, it is understood that, for a given compound, one atropisomer may be more potent as an inhibitor of KRAS (including G12C, G12D or G12V mutated forms of KRAS) than another atropisomer. For example, compounds of Formula (I) as described herein can exist in the form of atropisomers in which the conformation of the dichlorophenyl relative to the remainder of the molecule is as shown by the partial formulae Formula A or Formula B below. Atropisomeric forms may be isolable by methods such as chromatography. The stereochemistry of atropisomeric forms can be designated, e.g., as either Rₐ or Sₐ by applying IUPAC rules of nomenclature for stereochemistry. G. P. Moss, Pure & Appl. Chem., 1996, 68(12), 2193-2222. Without being limited by any theory, and by way of example, it is understood that the atropisomer represented by Formula (A) and (B), for example, may be separable, and one may be more potent as an inhibitor of KRAS (including G12C, G12D or G12V mutated forms of KRAS) than the other atropisomer atropisomer. In some embodiments, an atropisomer can be least partially or substantially separated from the alternative atropisomer of the compound, for example containing about 40% or less, about 30% or less, about 20% or less, about 10% or less, about 5% or less, about 2% or less, or about 1% or less of the alternative atropisomer.

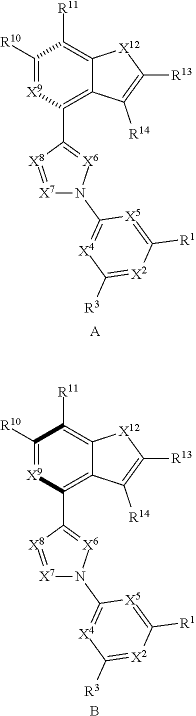

Compounds of the present disclosure can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the present disclosure can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. In the compounds provided herein, any atom not specifically designated as a particular isotope is meant to represent any stable isotope of that atom.

Substitution with heavier isotopes such as deuterium, may afford certain therapeutic advantages resulting from greater metabolic stability, for example, increased in vivo half-life or reduced dosage requirements, and hence may be preferred in some circumstances. (A. Kerekes et. al. J. Med. Chem. 2011, 54, 201-210; R. Xu et. al. J. Label Compd. Radiopharm. 2015, 58, 308-312). Unless otherwise stated, when a position is designated specifically as “D” or “deuterium,” the position is understood to have deuterium at an abundance that is at least 3000 times greater than the natural abundance of deuterium, which is 0.015% (i.e., at least 45% incorporation of deuterium). In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “compound” is intended, unless otherwise specified, to include all stereoisomers, including without limitation, geometric isomers, configurational isomers, conformational isomers, rotational isomers, and atropisomers, of the structures depicted, including each of the embodiments thereof. The term is also intended to refer to compounds described herein regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds provided herein, or salts thereof, are substantially isolated. “Substantially isolated” means that the compound is at least partially or substantially separated from the environment in which it was formed or detected. Partial separation can include, e.g., a composition enriched in the compounds of the present disclosure. Substantial separation can include compositions containing at least about 50%, at least about 60%, at least about 70%, at least about 80%, at least about 90%, at least about 95%, at least about 97%, or at least about 99% by weight of the compounds of the present disclosure, or salt thereof.

The phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions “ambient temperature” and “room temperature” are understood in the art, and refer generally to a temperature, e.g., a reaction temperature, that is about the temperature of the room in which the reaction is carried out, e.g., a temperature from about 20° C. to about 30° C.

The present disclosure also includes pharmaceutically acceptable salts of the compounds described herein, including any of the embodiments thereof. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, e.g., from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011). In some embodiments, the compounds described herein include the N-oxide forms.

### II. Synthesis

Compounds of the present disclosure, including salts thereof, can be prepared using known organic synthesis techniques and can be synthesized according to any of numerous possible synthetic routes, such as those in the schemes below.

The reactions for preparing compounds of the present disclosure can be carried out in suitable solvents which can be readily selected by one of skill in the art of organic synthesis. suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the present disclosure can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); smith et al., March's Advanced Organic Chemistry: Reactions, Mechanisms, and structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high-performance liquid chromatography (HPLC), liquid chromatography-mass spectroscopy (LCMs), or thin layer chromatography (TLC).

Compounds can be purified by those skilled in the art by a variety of methods, including high performance liquid chromatography (HPLC) (“Preparative LCMs Purification: Improved Compound specific Method Optimization” Karl F. Blom, Brian Glass, Richard sparks, Andrew P. Combs J. Combi. Chem. 2004, 6(6), 874-883) and normal phase silica chromatography.

The schemes below provide general guidance in connection with preparing the compounds of the present disclosure. One skilled in the art would understand that the preparations shown in the schemes can be modified or optimized using general knowledge of organic chemistry to prepare various compounds of the present disclosure.

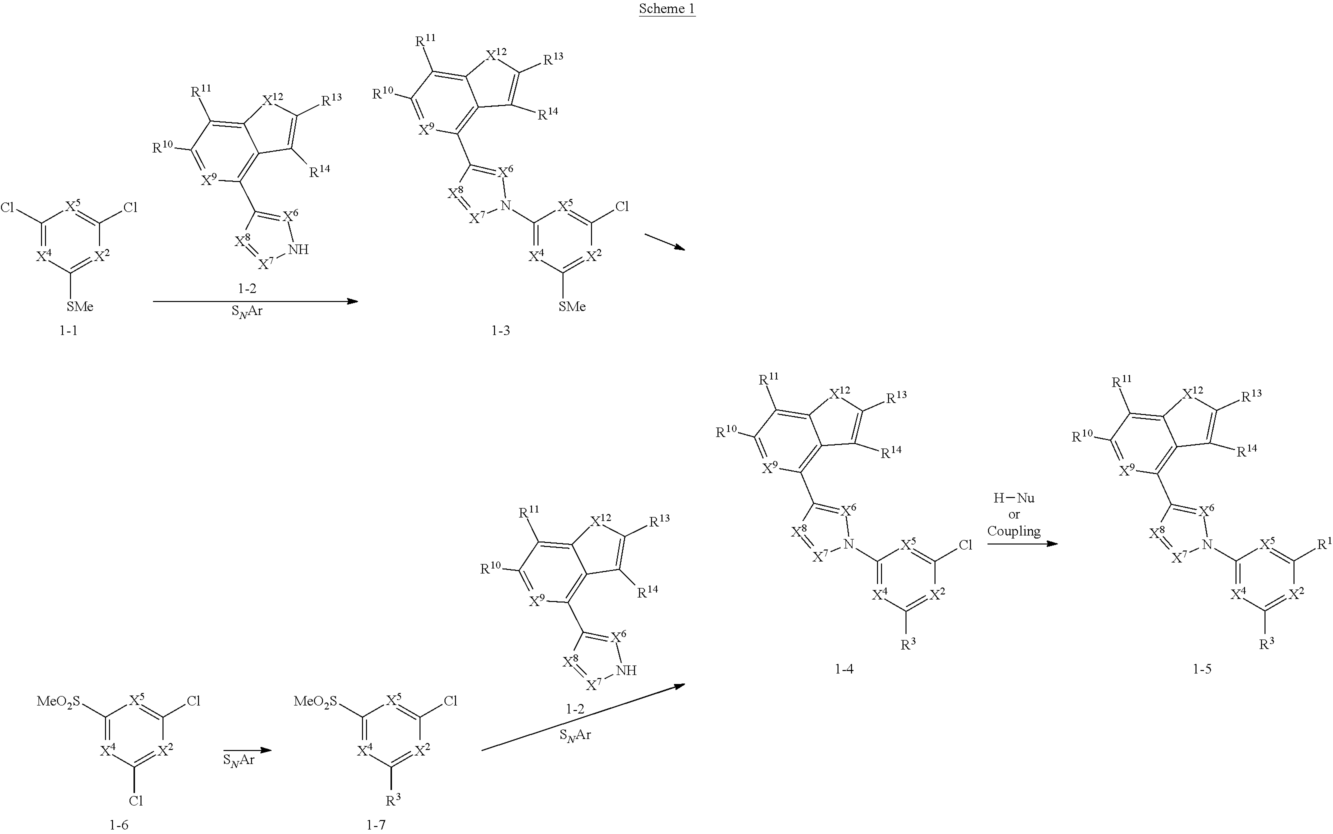

Compounds of formula 1-5 can be prepared via the synthetic routes outlined in Scheme 1. A nucleophilic aromatic substitution (S_{N}Ar) reaction of the chloro-substituted heterocycle 1-1 with a heterocyclic nucleophile 1-2 in the presence of a suitable base can deliver intermediate 1-3. Intermediate 1-3 can be converted to intermediate 1-4 either through oxidation of the thioether functional group with a suitable oxidant (e.g. mCPBA) followed by an S_{N}Ar reaction, or a cross-coupling reaction (Org. Lett. 2002, 4, 979-981). The chloro of Intermediate 1-4 can then be converted to R⁴ via a S_{N}Ar reaction, or standard Buchwald-Hartwig cross-coupling conditions (e.g., in the presence of a palladium catalyst and a suitable base), or standard Suzuki Cross-Coupling conditions (e.g., in the presence of a palladium catalyst and a suitable base), or standard Negishi Cross-Coupling conditions, or standard Stille Cross-Coupling conditions, or other suitable methods to obtain compound 1-5.

Alternatively, the synthesis sequence can begin with the chloro-substituted aromatic sulfone 1-6, which can be converted to intermediate 1-7 via a S_{N}Ar reaction. Another S_{N}Ar reaction of intermediate 1-7 with the heterocyclic nucleophile 1-2 can deliver intermediate 1-4, which can be further elaborated into compound 1-5 as described above.

Compounds of formula 2-7 can be prepared via the synthetic routes outlined in

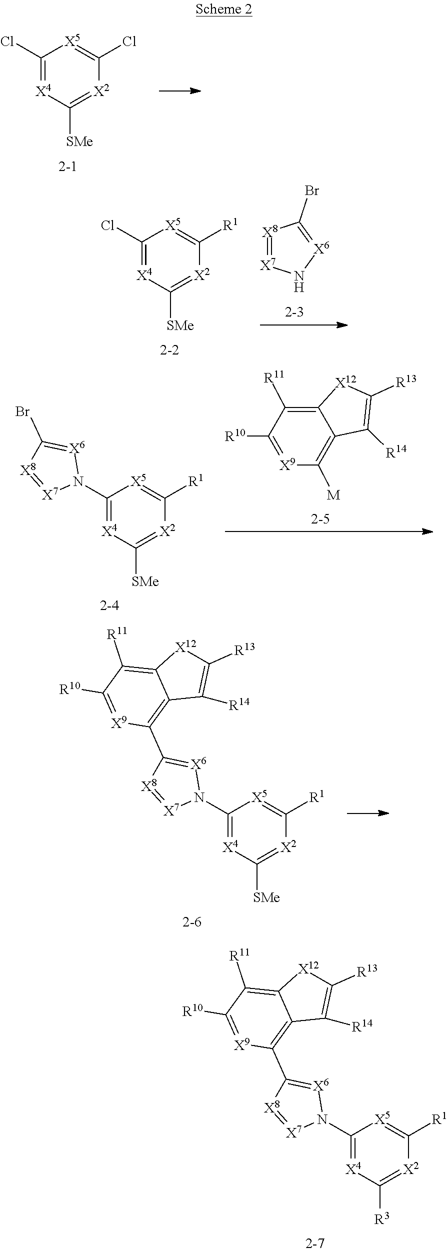

The chloro-substituted heterocycle 2-1 can be converted into intermediate 2-2 via a S_{N}Ar reaction, or standard Suzuki Cross-Coupling conditions (e.g., in the presence of a palladium catalyst and a suitable base), or standard Negishi Cross-Coupling conditions, or standard Stille Cross-Coupling conditions. The chloro of intermediate 2-2 can be substituted by a heterocyclic nucleophile 2-3 to deliver intermediate 2-4, which is further reacted with an organometallic reagent 2-5 via a standard Suzuki Cross-Coupling conditions (e.g., in the presence of a palladium catalyst and a suitable base), or standard Negishi Cross-Coupling conditions, or standard Stille Cross-Coupling conditions to afford intermediate 2-6. Compound 2-7 can be synthesized from intermediate 2-6 either through oxidation of the thioether functional group with a suitable oxidant (e.g. mCPBA) followed by an S_{N}Ar reaction, or a cross-coupling reaction (Org. Lett. 2002, 4, 979-981).

For the synthesis of particular compounds, the general schemes described above and specific methods described herein for preparing particular compounds can be modified. For example, the order of the above-described chemical reactions can be rearranged or omitted as appropriate to suit the preparation of different analogues, and the products or intermediates can be modified to introduce particular functional groups. Alternatively, the substituents can be modified at any step of the overall synthesis by methods know to one skilled in the art, e.g., as described by R. C. Larock, Comprehensive Organic Transformations: A Guide to Functional Group Preparations (Wiley, 1999); R. C. Larock, et al., Comprehensive Organic Transformations: A Guide to Functional Group Preparations, 3^{rd} Ed. Vols. 1-4 (Wiley, 2018); A. R. Katritzky, et al. (Eds.), Comprehensive Organic Functional Group Transformations, Vols. 1-6 (Pergamon Press, 1995), and A. R. Katritzky et al. (Eds.), Comprehensive Organic Functional Group Transformations II, Vols. 1-6 (Elsevier, 2^{nd} Edition, 2005).

Starting materials, reagents and intermediates whose synthesis is not described herein are either commercially available, known in the literature, or may be prepared by methods known to one skilled in the art.

It will be appreciated by one skilled in the art that the processes described are not the exclusive means by which compounds of the present disclosure may be synthesized and that a broad repertoire of synthetic organic reactions is available to be potentially employed in synthesizing compounds of the present disclosure. The person skilled in the art knows how to select and implement appropriate synthetic routes. suitable synthetic methods of starting materials, intermediates and products may be identified by reference to the literature, including reference sources such as: Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) science of synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier science, 2008); D. st.C. Black, et al. (Eds.); Comprehensive Heterocyclic Chemistry IV, Vols. 1-14 (Elsevier science, 2022); M. B. smith et al., March's Advanced Organic Chemistry: Reactions, Mechanisms, and structure, 6^{th} Ed. (Wiley, 2007); M. B. smith et al., March's Advanced Organic Chemistry: Reactions, Mechanisms, and structure, 8^{th} Ed. (Wiley, 2020); B. M. Trost et al. (Ed.), Comprehensive Organic synthesis, Vols. 1-9 (Pergamon Press, 1991); and Patai's Chemistry of Functional Groups, 100 Vols. (Wiley 1964-2022).

Separation of atropisomers can be carried out on the compounds of Formula (I) or any of the intermediates. The separating can be performed using chromatography such as by HPLC or supercritical fluid chromatography.

Starting materials, reagents and intermediates whose synthesis is not described herein are either commercially available, known in the literature, or may be prepared by methods known to one skilled in the art.

It will be appreciated by one skilled in the art that the processes described are not the exclusive means by which compounds of the present disclosure may be synthesized and that a broad repertoire of synthetic organic reactions is available to be potentially employed in synthesizing compounds of the present disclosure. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-114 (Elsevier, 1963-2023); Journal of Heterocyclic Chemistry Vols. 1-60 (Journal of Heterocyclic Chemistry, 1964-2023); E. M. Carreira, et al. (Eds.) Science of Synthesis, Vols. 1-48 (2001-2010) and Knowledge Updates KU2010/1-4; 2011/1-4; 2012/1-4, 2013/1-4; 2014/1-4, 2015/1-2; 2016/1-3, 2017/1-3; 2018/1-4, 2019/1-3; 2020/1-3, 2021/1-3, 2022/1-3, 2023/1 (Thieme, 2001-2023); Houben-Weyl, Methoden der Organischen Chemie, 4^{th} Ed. Vols. 1-67 (Thieme, 1952-1987); Houben-Weyl, Methoden der Organischen Chemie, E-Series. Vols. 1-23 (Thieme, 1982-2003); A. R. Katritzky, et al. (Eds.), Comprehensive Organic Functional Group Transformations, Vols. 1-6 (Pergamon Press, 1995); A. R. Katritzky et al. (Eds.), Comprehensive Organic Functional Group Transformations II, Vols. 1-6 (Elsevier, 2^{nd} Edition, 2005); A. R. Katritzky et al. (Eds.); Comprehensive Heterocyclic Chemistry, Vols. 1-8 (Pergamon Press, 1984); A. R. Katritzky, et al. (Eds.); Comprehensive Heterocyclic Chemistry II, Vols. 1-10 (Pergamon Press, 1996); A. R. Katritzky, et al. (Eds.); Comprehensive Heterocyclic Chemistry III, Vols. 1-14 (Elsevier Science, 2008); D. St.C. Black, et al. (Eds.); Comprehensive Heterocyclic Chemistry IV, Vols. 1-14 (Elsevier Science, 2022); M. B. Smith et al., March's Advanced Organic Chemistry: Reactions, Mechanisms, and Structure, 6^{th} Ed. (Wiley, 2007); M. B. Smith et al., March's Advanced Organic Chemistry: Reactions, Mechanisms, and Structure, 8^{th} Ed. (Wiley, 2020); B. M. Trost et al. (Ed.), Comprehensive Organic Synthesis, Vols. 1-9 (Pergamon Press, 1991); and Patai's Chemistry of Functional Groups, 100 Vols. (Wiley 1964-2022).

### III. Uses of the Compounds

Compounds of the present disclosure, including the compounds of Formula (I), or any of the embodiments thereof, are useful for therapy as described in further detail below. The present disclosure provides compounds of Formula (I), for use as a medicament, or for use in medicine. The present disclosure provides compounds of Formula (I), for use as a medicament, or for use in treating disease, as described in further detail below. The present disclosure also provides the use of compounds of Formula (I), or any of the embodiments thereof, as a medicament, or for treating disease, as described in further detail below. The present disclosure also provides the use of compounds of Formula (I), or any of the embodiments thereof, in the manufacture of medicament for treating disease, as described in further detail below.

Compounds of the present disclosure are KRAS inhibitors and, thus, are useful in treating diseases and disorders associated with activity of KRAS. For the uses described herein, any of the compounds of Formula (I), including any of the embodiments thereof, may be used.

In particular, compounds of the present disclosure are KRAS inhibitors having activity against one or more mutant forms of KRAS, and, thus, are useful in treating diseases and disorders associated with the presence or activity of mutant forms of KRAS, such as G12C, G12D, and/or the G12V mutant forms of KRAS.

The Ras family is comprised of three members: KRAS, NRAS and HRAS. RAS mutant cancers account for about 25% of human cancers. KRAS is the most frequently mutated isoform in human cancers: 85% of all RAS mutations are in KRAS, 12% in NRAS, and 3% in HRAS (D. Simanshu, et al., Cell, 2017, 170(1), 17-33). KRAS mutations are prevalent amongst the top three most deadly cancer types: pancreatic (97%), colorectal (44%), and lung (30%) (A. D. Cox, et al., Nat. Rev. Drug. Discov., 2014, 13(11), 828-51). The majority of RAS mutations occur at amino acid residues/codons 12, 13, and 61; Codon 12 mutations are most frequent in KRAS. The frequency of specific mutations varied between RAS genes and G12D mutations are most predominant in KRAS whereas Q61R and G12R mutations are most frequent in NRAS and HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) (A. D. Cox, et al., Nat. Rev. Drug. Discov., 2014, 13(11), 828-51). In contrast, KRAS G12C mutations predominate in non-small cell lung cancer (NSCLC) comprising 11-16% of lung adenocarcinomas (nearly half of mutant KRAS is G12C), as well as 2-5% of pancreatic and colorectal adenocarcinomas, respectively (A. D. Cox, et al., Nat. Rev. Drug. Discov., 2014, 13(11), 828-51). Using shRNA knockdown thousands of genes across hundreds of cancer cell lines, genomic studies have demonstrated that cancer cells exhibiting KRAS mutations are highly dependent on KRAS function for cell growth (R. McDonald, et al., Cell, 2017, 170(3), 577-92).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

Diseases that can be treated with the compounds of Formula (I) include cancers. The cancers can include adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor. In some embodiments, the cancer can be adenocarcinoma, adult T-cell leukemia/lymphoma, bladder cancer, blastoma, bone cancer, breast cancer, brain cancer, carcinoma, myeloid sarcoma, cervical cancer, colorectal cancer, esophageal cancer, gastrointestinal cancer, glioblastoma multiforme, glioma, gallbladder cancer, gastric cancer, head and neck cancer, Hodgkin's lymphoma, non-Hodgkin's lymphoma, intestinal cancer, kidney cancer, laryngeal cancer, leukemia, lung cancer, lymphoma, liver cancer, small cell lung cancer, non-small cell lung cancer, mesothelioma, multiple myeloma, ocular cancer, optic nerve tumor, oral cancer, ovarian cancer, pituitary tumor, primary central nervous system lymphoma, prostate cancer, pancreatic cancer, pharyngeal cancer, renal cell carcinoma, rectal cancer, sarcoma, skin cancer, spinal tumor, small intestine cancer, stomach cancer, T-cell lymphoma, testicular cancer, thyroid cancer, throat cancer, urogenital cancer, urothelial carcinoma, uterine cancer, vaginal cancer, or Wilms' tumor.

The cancer types in which KRAS harboring G12C, G12V and G12D mutations are implicated and that can be treated using compounds of Formula (I), or any of the embodiments thereof, include, but are not limited to: carcinomas (e.g., pancreatic, colorectal, lung, bladder, gastric, esophageal, breast, head and neck, cervical skin, thyroid); hematopoietic malignancies (e.g., myeloproliferative neoplasms (MPN), myelodysplastic syndrome (MDS), chronic and juvenile myelomonocytic leukemia (CMML and JMML), acute myeloid leukemia (AML), acute lymphocytic leukemia (ALL) and multiple myeloma (MM)); and other neoplasms (e.g., glioblastoma and sarcomas). In addition, KRAS mutations were found in acquired resistance to anti-EGFR therapy (K. Knickelbein, et al., Genes Dis., 2015, 2(1), 4-12). KRAS mutations were found in immunological and inflammatory disorders (A. Fernandez-Medarde, et al., Genes Cancer, 2011, 2(3), 344-358) such as Ras-associated lymphoproliferative disorder (RALD) or juvenile myelomonocytic leukemia (JMML) caused by somatic mutations of KRAS or NRAS.

Compounds of the present disclosure, including any of the embodiments thereof, can inhibit the activity of the KRAS protein. For example, compounds of the present disclosure can be used to inhibit activity of KRAS in a cell or in an individual or patient in need of inhibition of the enzyme by administering an inhibiting amount of one or more compounds of the present disclosure to the cell, individual, or patient.

As KRAS inhibitors, the compounds of the present disclosure, or any of the embodiments thereof, are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compounds of the present disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In an aspect, provided herein is a method of inhibiting KRAS activity, the method comprising contacting a compound of the instant disclosure with KRAS. In some embodiments, the contacting comprises administering the compound to a patient. In some embodiments, KRAS is characterized as having a somatic mutation of G12C. In other embodiments, KRAS is characterized as having a somatic mutation of G12D. In other embodiments, KRAS is characterized as having a somatic mutation of G12V.

In an aspect, provided herein is a method of inhibiting a KRAS protein harboring a G12C mutation, the method comprising contacting a compound of Formula (I), or any of the embodiments thereof, with KRAS.

In an aspect, provided herein is a method of inhibiting a KRAS protein harboring a G12D mutation, the method comprising contacting a compound of Formula (I), or any of the embodiments thereof, with KRAS harboring a G12D mutation.

In an aspect, provided herein is a method of inhibiting a KRAS protein harboring a G12V mutation, the method comprising contacting a compound of Formula (I), or any of the embodiments thereof, with KRAS harboring a G12V mutation.

In another aspect, provided herein is a method of treating a disease or disorder associated with inhibition of KRAS interaction, the method comprising administering to a patient in need thereof a therapeutically effective amount of a compound of Formula (I), or any of the embodiments thereof.

In some embodiments, the disease or disorder is an immunological or inflammatory disorder. In other embodiments, the immunological or inflammatory disorder is Ras-associated lymphoproliferative disorder or juvenile myelomonocytic leukemia caused by somatic mutations of KRAS. In some embodiments, the immunological or inflammatory disorder is caused by a somatic mutation of KRAS.

In other embodiments, the somatic mutation of KRAS is G12C. In other embodiments, the somatic mutation of KRAS is G12D. In other embodiments, the somatic mutation of KRAS is G12V.

In other embodiments, the immunological or inflammatory disorder is associated with activity of KRAS having a G12C mutation. In other embodiments, the immunological or inflammatory disorder is associated with activity of KRAS having a G12D mutation. In other embodiments, the immunological or inflammatory disorder is associated with activity of KRAS having a G12V mutation.

In yet another aspect, provided herein is a method of treating a disease or disorder associated with inhibiting a KRAS protein harboring a G12C mutation, the method comprising administering to a patient in need thereof a therapeutically effective amount of a compound of a compound of Formula (I), or any of the embodiments thereof,

In yet another aspect, provided herein is a method of treating a disease or disorder associated with inhibiting a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with inhibiting a KRAS protein harboring a G12V mutation, the method comprising administering to a patient in need thereof a therapeutically effective amount of a compound of Formula (I), or any of the embodiments thereof.

In yet another aspect, provided herein is a method of treating a disease or disorder associated with activity of a KRAS protein harboring a G12C mutation, the method comprising administering to a patient in need thereof a therapeutically effective amount of a compound of a compound of Formula (I), or any of the embodiments thereof,

In yet another aspect, provided herein is a method of treating a disease or disorder associated with activity of a KRAS protein harboring a G12D mutation, the method comprising administering to a patient in need thereof a therapeutically effective amount of a compound of Formula (I), or any of the embodiments thereof.

In another aspect, provided herein is a method of treating a disease or disorder associated with activity of a KRAS protein harboring a G12V mutation, the method comprising administering to a patient in need thereof a therapeutically effective amount of a compound of Formula (I), or any of the embodiments thereof.

In another aspect, provided herein is also a method of treating cancer in a patient in need thereof comprising administering to the patient a therapeutically effective amount of a compound of Formula (I), or any of the embodiments thereof.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12C mutation.

In still another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12D mutation.

In another aspect, provided herein is also a method of treating cancer in a patient in need thereof comprising administering to the patient a therapeutically effective amount of a compound of Formula (I), or any of the embodiments thereof, wherein the cancer is characterized by an interaction with a KRAS protein harboring a G12V mutation.

In yet another aspect, provided herein is a method for treating a cancer in a patient, the method comprising administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12C mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12D mutation, and administering to the patient a therapeutically effective amount of any one of the compounds disclosed herein, or pharmaceutically acceptable salt thereof.

In another aspect, provided herein is a method for treating a cancer in a patient comprising identifying that the patient is in need of treatment of a cancer and that abnormally proliferating cells of the cancer comprise KRAS having a G12V mutation, and administering to the patient a therapeutically effective amount of any one of the compounds disclosed herein, or pharmaceutically acceptable salt thereof.

In some embodiments, the cancer is selected from carcinomas, hematological cancers, sarcomas, and glioblastoma. In other embodiments, the hematological cancer is selected from myeloproliferative neoplasms, myelodysplastic syndrome, chronic and juvenile myelomonocytic leukemia, acute myeloid leukemia, acute lymphocytic leukemia, and multiple myeloma. In yet other embodiments, the carcinoma is selected from pancreatic, colorectal, lung, bladder, gastric, esophageal, breast, head and neck, cervical, skin, and thyroid. In other embodiments, the cancer is colorectal cancer, pancreatic cancer, or lung cancer. In yet other embodiments, the pancreatic cancer is pancreatic ductal adenocarcinoma (PDAC). In still other embodiments, the cancer is non-small cell lung cancer (NSCLC).

In some embodiments, the cancer is metastatic.

In an aspect, provided herein is a method for treating a disease or disorder associated with inhibition of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In an aspect, provided herein is a method for treating a disease or disorder associated with activity of KRAS interaction or a mutant thereof, in a patient in need thereof, comprising the step of administering to the patient a compound disclosed herein, or a pharmaceutically acceptable salt thereof, or a composition comprising a compound disclosed herein or a pharmaceutically acceptable salt thereof, in combination with another therapy or therapeutic agent as described herein.

In some embodiments, the cancer is selected from hematological cancers, sarcomas, lung cancers, gastrointestinal cancers, genitourinary tract cancers, liver cancers, bone cancers, nervous system cancers, gynecological cancers, and skin cancers.

In other embodiments, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet other embodiments, the lung cancer is non-small cell lung cancer (NSCLC). In still other embodiments, the lung cancer is adenocarcinoma.

In some embodiments, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In some embodiments, the gastrointestinal cancer is colorectal cancer.

In other embodiments, the cancer is a carcinoma. In yet other embodiments, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still other embodiments, the cancer is a hematopoietic malignancy. In some embodiments, the hematopoietic malignancy is selected from multiple myeloma, acute myelogenous leukemia, and myeloproliferative neoplasms.

In other embodiments, the cancer is a neoplasm. In yet other embodiments, the neoplasm is glioblastoma or sarcomas.

In certain embodiments, the disclosure provides a method for treating a KRAS-mediated disorder in a patient in need thereof, comprising the step of administering to the patient a compound according to the present disclosure, or a pharmaceutically acceptable composition thereof.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome, myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compounds of the present disclosure can also be useful in the inhibition of tumor metastasis.

In addition to oncogenic neoplasms, the compounds of the present disclosure are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a patient suffering from a skeletal and chondrocyte disorder.

In some embodiments, compounds described herein can be used to treat Alzheimer's disease, HIV, or tuberculosis.

The term “8p11 myeloproliferative syndrome” refers to myeloid/lymphoid neoplasms associated with eosinophilia and abnormalities of FGFR1.

The term “cell” refers to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

The term “contacting” refers to the bringing together of indicated moieties in an in vitro system or an in vivo system. For example, “contacting” KRAS with a compound described herein includes the administration of a compound described herein to an individual or patient, such as a human, having KRAS, as well as, for example, introducing a compound described herein into a sample containing a cellular or purified preparation containing KRAS.

The terms “individual,” “subject,” or “patient,” are used interchangeably, and refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., P. Beringer, et al., (Eds.), Remington: The Science and Practice of Pharmacy, 21^{st} Ed.; (Lippincott Williams & Wilkins: Philadelphia, Pa., 2005); A. Adejare (Ed.), Remington, The Science and Practice of Pharmacy, 23^{rd} Ed., (Elsevier, 2020); R. C. Rowe et al., Eds., Handbook of Pharmaceutical Excipients, 6^{th} Ed.; (Pharmaceutical Press, 2009); P. J. Shesky et al., Eds., Handbook of Pharmaceutical Excipients, 9^{th} Ed.; (The Pharmaceutical Press, 2020); M. Ash, et al., (Eds.), Handbook of Pharmaceutical Additives, 3^{rd} Ed.; (Gower Publishing Company: 2007); and M. Gibson (Ed.), Pharmaceutical Preformulation and Formulation, 2^{nd} Ed. (CRC Press LLC, 2009).

The term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” comprises the prevention of at least one symptom associated with or caused by the state, disease or disorder being prevented.

### III. Combination Therapies

Compounds of the present disclosure, including the compounds of Formula (I), or any of the embodiments thereof, may be useful in therapy when used in combination with one or more additional pharmaceutical agents, as described in further detail below.

a. Cancer Therapies

Compounds of the present disclosure, including the compounds of Formula (I), or any of the embodiments thereof, may be useful in treatment of cancer when used in combination with one or more additional pharmaceutical agents, as described in further detail below.

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of KRAS-associated diseases, disorders or conditions. Other agents such as therapeutic antibodies can be used in combination with the compounds of the present disclosure for treatment of KRAS-associated diseases, disorders or conditions. The one or more additional pharmaceutical agents can be administered to a patient simultaneously or sequentially.

In some embodiments, the KRAS inhibitor is administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGF3R, P13K (alpha, beta, gamma, delta, and multiple or selective), CSF1R, KIT, FLK-II, KDR/FLK-1, FLK-4, flt-1, FGFR1, FGFR2, FGFR3, FGFR4, c-Met, PARP, Ron, Sea, TRKA, TRKB, TRKC, TAM kinases (Axi, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2; e.g., ruxolitinib or baricitinib; or JAK1; e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a P13K-delta inhibitor (e.g., parsaclisib (INCB50465) or INCB50797), a P13K-gamma inhibitor such as P13K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., zilurgisertib); or combinations thereof.

In some embodiments, the compound or salt described herein is administered with a P13K6 inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-1 agonists, oncolytic virotherapy and immunomodulating small molecules, including thalidomide or JAK1/2 inhibitor, P13K6 inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Pat. No. 7,745,437.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, B1853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK2 inhibitor of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the embodiments thereof as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the embodiments thereof as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limit to human papillomavirus, influenza, hepatitis A, B, C or D viruses, adenovirus, poxyirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

b. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, P13K delta, P13K gamma, TAM, arginase, HPK1, CD137 (also known as 4-1 BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1 BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (1B1308), AB122 (GLS-010), AMP-224, AMP-514/MEDI-0680, BMS936559, JTX-4014, BGB-108, SHR-1210, MED14736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217,149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550.

In some embodiments, the PD-L1 inhibitor is selected from the compounds in Table 3, or a pharmaceutically acceptable salt thereof.

TABLE 3

| | US | |
|---|---|---|
| Cmpd | Publication | |
| No. | Appl. No. | Name and Structure |
| 1 | US 2018- | (R)-1-((7-cyano-2-(3′-(3-(((R)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example | yl)methyl)pyrrolidine-3-carboxylic acid |
| | #24 | |
| | | 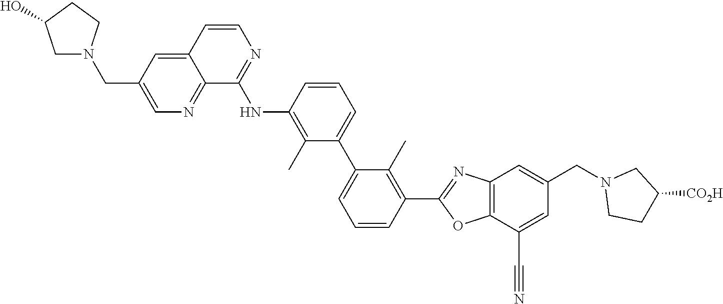 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C] |
| 2 | US 2018- | N-(2-chloro-3′-(8-chloro-6-((2-hydroxyethylamino)methyl)-[1,2,4]triazolo[1,5- |
| | 0179201, | alpyridin-2-yl)-2′-methylbiphenyl-3-yl)-5-((2- |
| | Example #2 | hydroxyethylamino)methyl)picolinamide |
| | | 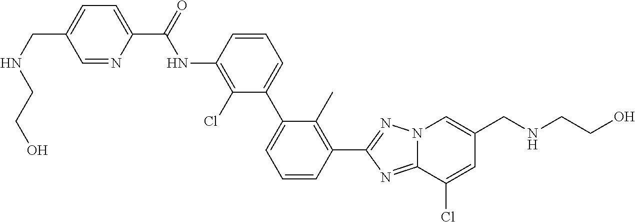 [SMILES: Cc1c(-c2nc3c(Cl)cc(CNCCO)cn3n2)cccc1-c1cccc(NC(=O)c2ccc(CNCCO)cn2)c1Cl] |
| 3 | US 2018- | (S)-1-((7-cyano-2-(3′-(3-(((S)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example | yl)methyl)pyrrolidine-3-carboxylic acid |
| | #25 | |
| | | 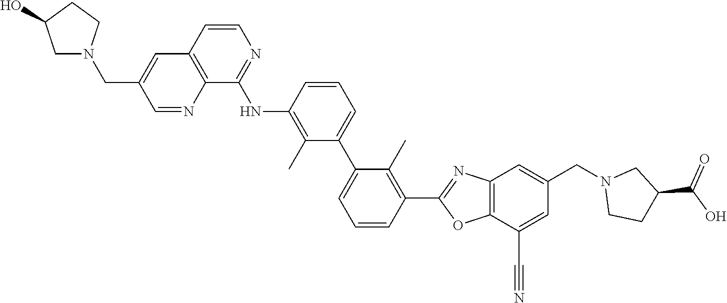 [SMILES: Cc1c(Nc2nccc3cc(CN4CC[C@H](O)C4)cnc23)cccc1-c1cccc(-c2nc3cc(CN4CC[C@H](C(=O)O)C4)cc(C#N)c3o2)c1C] |
| 4 | US 2018- | (R)-1-((7-cyano-2-(3′-(3-(((S)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example | yl)methyl)pyrrolidine-3-carboxylic acid |
| | #26 | |
| | | 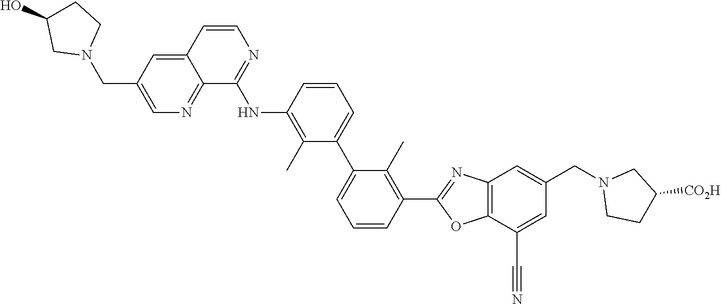 [SMILES: Cc1c(Nc2nccc3cc(CN4CC[C@H](O)C4)cnc23)cccc1-c1cccc(-c2nc3cc(CN4CC[C@@H](C(=O)O)C4)cc(C#N)c3o2)c1C] |
| 5 | US 2018- | (S)-1-((7-cyano-2-(3′-(3-(((R)-3-hydroxypyrrolidin-1-yl)methyl)-1,7- |
| | 0179197, | naphthyridin-8-ylamino)-2,2′-dimethylbiphenyl-3-yl)benzo[d]oxazol-5- |
| | Example | yl)methyl)pyrrolidine-3-carboxylic acid |
| | #28 | |
| | | 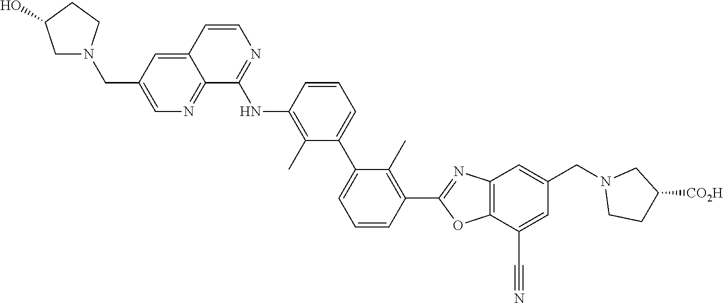 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C] |
| 6 | US 2018- | 1-((7-cyano-2-(3′-(5-(2-(dimethylamino)acetyl)-5,6-dihydro-4H-pyrrolo[3,4- |
| | 0179197, | d]thiazol-2-yl)-2,2′-dimethylbiphenyl-3-yl)benzo[d]oxazol-5- |
| | Example | yl)methyl)piperidine-4-carboxylic acid |
| | #236 | |
| | | 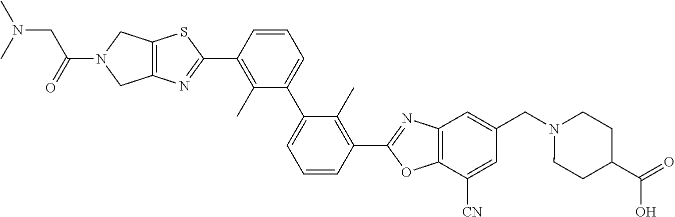 [SMILES: Cc1c(-c2nc3cc(CN4CCC(C(=O)O)CC4)cc(C#N)c3o2)cccc1-c1cccc(-c2nc3c(s2)CN(C(=O)CN(C)C)C3)c1C] |
| 7 | US 2018- | N,N-(2-chloro-2′-methylbiphenyl-3,3′-diyl)bis(5-((2-hydroxyethylamino) |
| | 0179179, | methyl)picolinamide) |
| | Example | |
| | #1 | 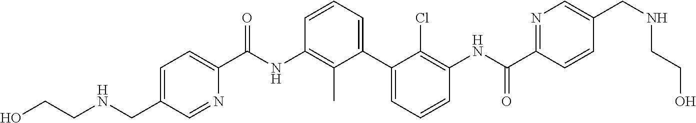 [SMILES: Cc1c(NC(=O)c2ccc(CNCCO)cn2)cccc1-c1cccc(NC(=O)c2ccc(CNCCO)cn2)c1Cl] |
| 8 | US 2018- | (R)-1-((6-(2′-chloro-3′-(5-((3-hydroxypyrrolidin-1-yl)methyl)picolinamido)-2- |
| | 0179179, | methylbiphenyl-3-ylcarbamoyl)pyridin-3-yl)methyl)piperidine-4-carboxylic acid |
| | Example | |
| | #9 | 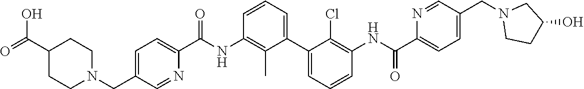 [SMILES: Cc1c(NC(=O)c2ccc(CN3CCC(C(=O)O)CC3)cn2)cccc1-c1cccc(NC(=O)c2ccc(CN3CC[C@@H](O)C3)cn2)c1Cl] |
| 9 | US 2018- | (S)-1-((6-((2′-chloro-2-methyl-3′-(5-(pyrrolidin-1-ylmethyl)picolinamido)-[1,1′- |
| | 0179179, | biphenyl]-3-yl)carbamoyl)-4-methylpyridin-3-yl)methyl)piperidine-2-carboxylic |
| | Example | acid |
| | #12 | |
| | | 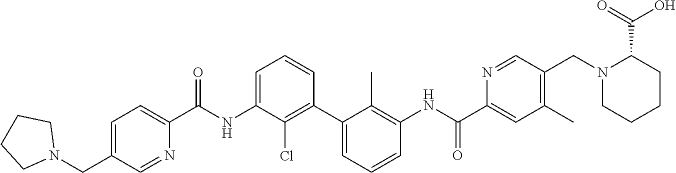 [SMILES: Cc1cc(C(=O)Nc2cccc(-c3cccc(NC(=O)c4ccc(CN5CCCC5)cn4)c3Cl)c2C)ncc1CN1CCCC[C@H]1C(=O)O] |
| 10 | US 2018- | trans 4-(2-(2-(2-chloro-3′-(3-(((R)-3-hydroxypyrrolidin-1-yl)methyl)-1,7- |
| | 0179202, | naphthyridin-8-ylamino)-2′-methylbiphenyl-3-ylcarbamoyl)-1-methyl-6,7- |
| | Example | dihydro-1H-imidazo[4,5-c]pyridin-5(4H)-yl)ethyl)cyclohexanecarboxylic acid |
| | #52 | |
| | | 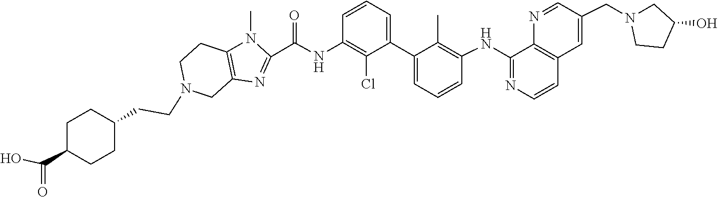 [SMILES: Cc1c(Nc2nccc3cc(CN4CC[C@@H](O)C4)cnc23)cccc1-c1cccc(NC(=O)c2nc3c(n2C)CCN(CC[C@H]2CC[C@H](C(=O)O)CC2)C3)c1Cl] |
| 11 | US 2018- | cis-4-((2-(2-chloro-3′-(3-(((R)-3-hydroxy-3-methylpyrrolidin-1-yl)methyl)-1,7- |
| | 0179202, | naphthyridin-8-ylamino)-2′-methylbiphenyl-3-ylcarbamoyl)-1-methyl-6,7- |
| | Example | dihydro-1H-imidazo[4,5-c]pyridin-5(4H)-yl)methyl)cyclohexanecarboxylic acid |
| | #56 | |
| | | 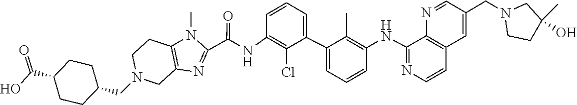 [SMILES: Cc1c(Nc2nccc3cc(CN4CC[C@@](C)(O)C4)cnc23)cccc1-c1cccc(NC(=O)c2nc3c(n2C)CCN(C[C@H]2CC[C@@H](C(=O)O)CC2)C3)c1Cl] |
| 12 | US 2018- | (R)-4-(2-(2-chloro-3′-(7-((3-hydroxypyrrolidin-1-yl)methyl)pyrido[3,2- |
| | 0179202, | d]pyrimidin-4-ylamino)-2′-methylbiphenyl-3-ylcarbamoyl)-1-methyl-6,7- |
| | Example | dihydro-1H-imidazo[4,5-c]pyridin-5(4H)-yl)-1-methylcyclohexanecarboxylic |
| | #68 | acid |
| | | 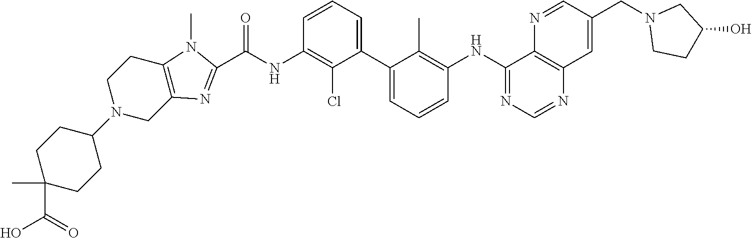 [SMILES: Cc1c(Nc2ncnc3cc(CN4CC[C@@H](O)C4)cnc23)cccc1-c1cccc(NC(=O)c2nc3c(n2C)CCN(C2CCC(C)(C(=O)O)CC2)C3)c1Cl] |
| 13 | US 2018- | (R)-1-((8-((2-chloro-3′-(5-(N-ethyl-N-methylglycyl)-5,6-dihydro-4H-pyrrolo[3,4- |
| | 0179202, | d]thiazol-2-yl)-2′-methyl-[1,1′-biphenyl]-3-yl)amino)-1,7-naphthyridin-3- |
| | Example | yl)methyl)pyrrolidine-3-carboxylic acid |
| | #90 | |
| | | 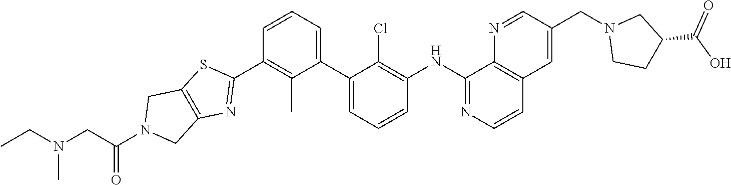 [SMILES: CCN(C)CC(=O)N1Cc2nc(-c3cccc(-c4cccc(Nc5nccc6cc(CN7CC[C@@H](C(=O)O)C7)cnc56)c4Cl)c3C)sc2C1] |
| 14 | US 2018- | (R)-2-(dimethylamino)-1-(2-(3′-(5-(2-(3-hydroxypyrrolidin-1-yl)acetyl)-5,6- |
| | 0177784, | dihydro-4H-pyrrolo[3,4-d]thiazol-2-yl)-2,2′-dimethylbiphenyl-3-yl)-4H- |
| | Example | pyrrolo[3,4-d]thiazol-5(6H)-yl)ethanone |
| | #35 | |
| | | 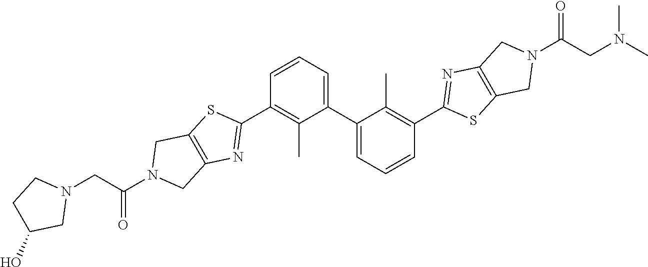 [SMILES: Cc1c(-c2nc3c(s2)CN(C(=O)CN(C)C)C3)cccc1-c1cccc(-c2nc3c(s2)CN(C(=O)CN2CC[C@@H](O)C2)C3)c1C] |
| 15 | US 2018- | trans-4-((2-(2′-chloro-3′-(1,5-dimethyl-4,5,6,7-tetrahydro-1H-imidazo[4,5- |
| | 0177870, | c]pyridine-2-carboxamido)-2-methylbiphenyl-3-ylcarbamoyl)-1-methyl-6,7- |
| | Example | dihydro-1H-imidazo[4,5-c]pyridin-5(4H)-yl)methyl)cyclohexane-1-carboxylic |
| | #37 | acid |
| | | 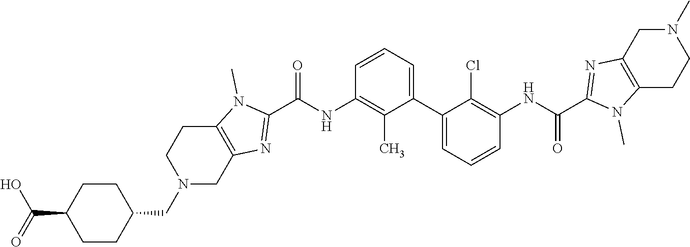 [SMILES: Cc1c(NC(=O)c2nc3c(n2C)CCN(C[C@H]2CC[C@H](C(=O)O)CC2)C3)cccc1-c1cccc(NC(=O)c2nc3c(n2C)CCN(C)C3)c1Cl] |
| 16 | US 2018- | trans-4-(2-(2-((2′-chloro-3′-(1,5-dimethyl-4,5,6,7-tetrahydro-1H-imidazo[4,5- |
| | 0177870, | c]pyridine-2-carboxamido)-2-methyl-[1,1′-biphenyl]-3-yl)carbamoyl)-1-methyl- |
| | Example | 1,4,6,7-tetrahydro-5H-imidazo[4,5-c]pyridin-5-yl)ethyl)cyclohexane-1- |
| | #100 | carboxylic acid |
| | | 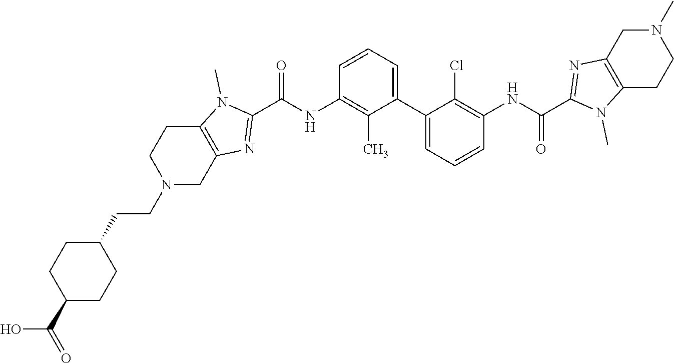 [SMILES: Cc1c(NC(=O)c2nc3c(n2C)CCN(CC[C@H]2CC[C@H](C(=O)O)CC2)C3)cccc1-c1cccc(NC(=O)c2nc3c(n2C)CCN(C)C3)c1Cl] |
| 17 | US 2018- | cis-4-((2-((2′-chloro-3′-(1,5-dimethyl-4,5,6,7-tetrahydro-1H-imidazo[4,5- |
| | 0177870, | c]pyridine-2-carboxamido)-2-methyl-[1,1′-biphenyl]-3-yl)carbamoyl)-1-methyl- |
| | Example | 1,4,6,7-tetrahydro-5H-imidazo[4,5-c]pyridin-5-yl)methyl)cyclohexane-1- |
| | #114 | carboxylic acid |
| | | 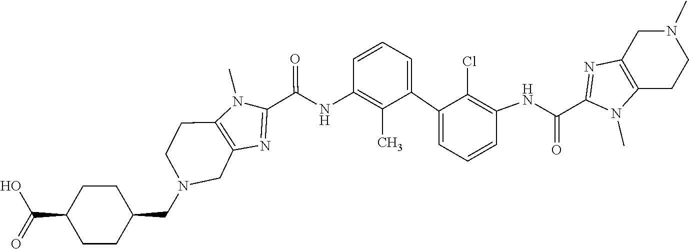 [SMILES: Cc1c(NC(=O)c2nc3c(n2C)CCN(C[C@H]2CC[C@@H](C(=O)O)CC2)C3)cccc1-c1cccc(NC(=O)c2nc3c(n2C)CCN(C)C3)c1Cl] |
| 18 | US 2018- | cis-4-((2-((2-chloro-3′-(1,5-dimethyl-4,5,6,7-tetrahydro-1H-imidazo[4,5- |
| | 0177870, | c]pyridine-2-carboxamido)-2′-methyl-[1,1′-biphenyl]-3-yl)carbamoyl)-1-methyl- |
| | Example | 1,4,6,7-tetrahydro-5H-imidazo[4,5-c]pyridin-5-yl)methyl)cyclohexane-1- |
| | #135 | carboxylic acid |
| | | 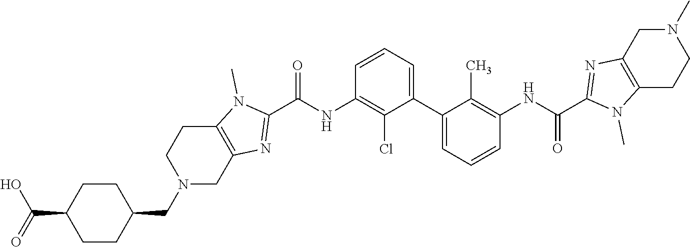 [SMILES: Cc1c(NC(=O)c2nc3c(n2C)CCN(C)C3)cccc1-c1cccc(NC(=O)c2nc3c(n2C)CCN(C[C@H]2CC[C@@H](C(=O)O)CC2)C3)c1Cl] |
| 19 | US 2018- | trans-4-(2-(2-((2′-chloro-2-cyano-3′-(1,5-dimethyl-4,5,6,7-tetrahydro-1H- |
| | 0177870, | imidazo[4,5-c]pyridine-2-carboxamido)-[1,1′-biphenyl]-3-yl)carbamoyl)-1- |
| | Example | methyl-1,4,6,7-tetrahydro-5H-imidazo[4,5-c]pyridin-5-yl)ethyl)cyclohexane-1- |
| | #148 | carboxylic acid |
| | | 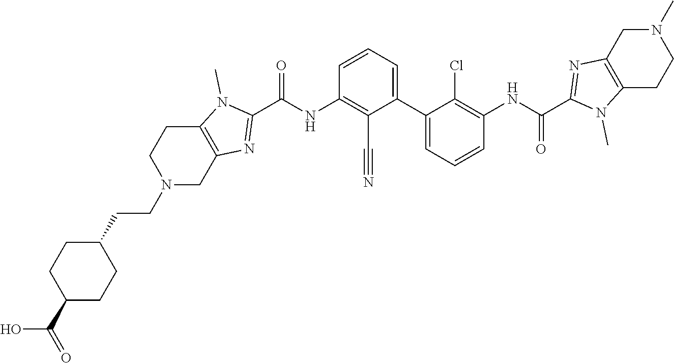 [SMILES: CN1CCc2c(nc(C(=O)Nc3cccc(-c4cccc(NC(=O)c5nc6c(n5C)CCN(CC[C@H]5CC[C@H](C(=O)O)CC5)C6)c4C#N)c3Cl)n2C)C1] |
| 20 | US 2018- | trans-4-((2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)methyl)cyclohexane-1-carboxylic |
| | #159 | acid |
| | | 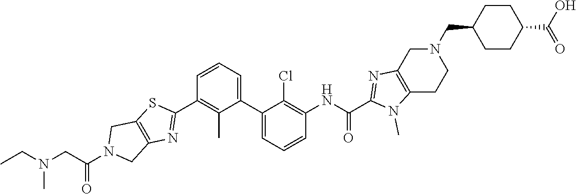 [SMILES: CCN(C)CC(=O)N1Cc2nc(-c3cccc(-c4cccc(NC(=O)c5nc6c(n5C)CCN(C[C@H]5CC[C@H](C(=O)O)CC5)C6)c4Cl)c3C)sc2C1] |
| 21 | US 2018- | cis-4-((2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)methyl)cyclohexane-1-carboxylic |
| | #160 | acid |
| | | 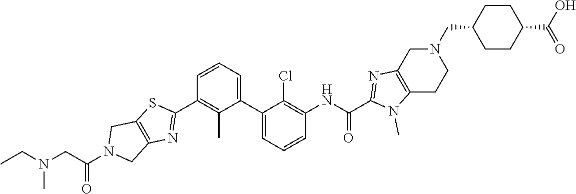 [SMILES: CCN(C)CC(=O)N1Cc2nc(-c3cccc(-c4cccc(NC(=O)c5nc6c(n5C)CCN(C[C@H]5CC[C@@H](C(=O)O)CC5)C6)c4Cl)c3C)sc2C1] |
| 22 | US 2018- | 4-(2-(2-(2-chloro-3′-(5-(2-(ethyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)ethyl)cyclohexane-1-carboxylic |
| | #161 | acid |
| | | 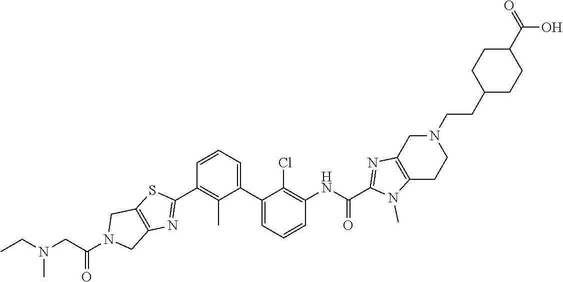 [SMILES: CCN(C)CC(=O)N1Cc2nc(-c3cccc(-c4cccc(NC(=O)c5nc6c(n5C)CCN(CCC5CCC(C(=O)O)CC5)C6)c4Cl)c3C)sc2C1] |
| 23 | US 2018- | 4-(2-(2-(2-chloro-3′-(5-(2-(isopropyl(methyl)amino)acetyl)-5,6-dihydro-4H- |
| | 0177870, | pyrrolo[3,4-d]thiazol-2-yl)-2′-methylbiphenyl-3-ylcarbamoyl)-1-methyl-6,7- |
| | Example | dihydro-1H-imidazo[4,5-c]pyridin-5(4H)-yl)ethyl)cyclohexane-1-carboxylic |
| | #162 | acid |
| | | 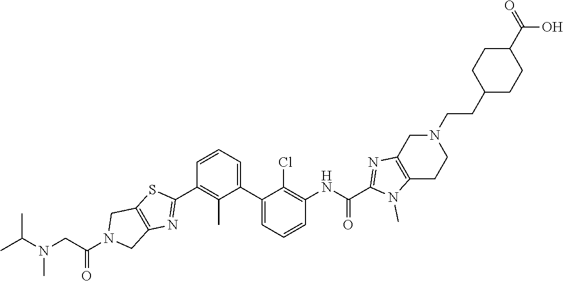 [SMILES: Cc1c(-c2nc3c(s2)CN(C(=O)CN(C)C(C)C)C3)cccc1-c1cccc(NC(=O)c2nc3c(n2C)CCN(CCC2CCC(C(=O)O)CC2)C3)c1Cl] |
| 24 | US 2019- | (R)-1-((7-cyano-2-(3′-(2-(difluoromethyl)-7-((3-hydroxypyrrolidin-1- |
| | 0300524, | yl)methyl)pyrido[3,2-d]pyrimidin-4-ylamino)-2,2′-dimethylbiphenyl-3- |
| | Example | yl)benzo[d]oxazol-5-yl)methyl)piperidine-4-carboxylic acid |
| | #16 | |
| | | 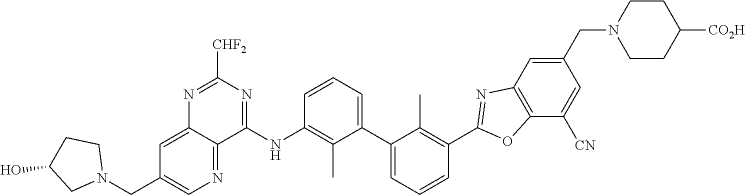 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C] |
| 25 | US 2019- | (R)-1-((7-cyano-2-(3′-(2-(difluoromethyl)-7-(((R)-3-hydroxypyrrolidin-1- |
| | 0300524, | yl)methyl)pyrido[3,2-d]pyrimidin-4-ylamino)-2,2′-dimethylbiphenyl-3- |
| | Example | yl)benzo[d]oxazol-5-yl)methyl)pyrrolidine-3-carboxylic acid |
| | #17 | |
| | | 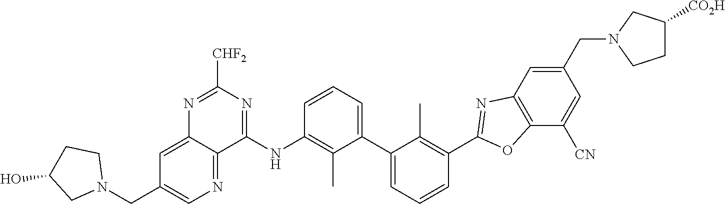 [SMILES: Cc1c(Nc2nc(C(F)F)nc3cc(CN4CC[C@@H](O)C4)cnc23)cccc1-c1cccc(-c2nc3cc(CN4CC[C@@H](C(=O)O)C4)cc(C#N)c3o2)c1C] |
| 26 | US 2019- | (R)-1-((7-cyano-2-(3′-(2-(difluoromethyl)-7-(((R)-3-hydroxypyrrolidin-1- |
| | 0300524, | yl)methyl)pyrido[3,2-d]pyrimidin-4-ylamino)-2,2′-dimethylbiphenyl-3- |
| | Example | yl)benzo[d]oxazol-5-yl)methyl)-3-methylpyrrolidine-3-carboxylic acid |
| | #18 | |
| | | 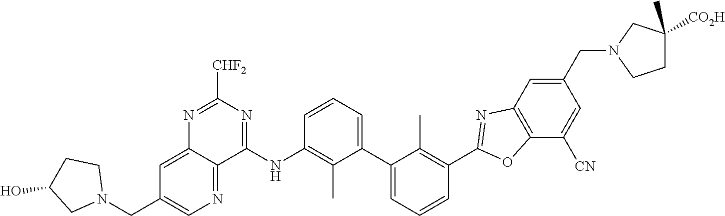 [SMILES: Cc1c(Nc2nc(C(F)F)nc3cc(CN4CC[C@@H](O)C4)cnc23)cccc1-c1cccc(-c2nc3cc(CN4CC[C@@](C)(C(=O)O)C4)cc(C#N)c3o2)c1C] |
| 27 | US 2019- | (R)-1-((7-cyano-2-(3′-(2-(difluoromethyl)-7-((3-hydroxy-3-methylpyrrolidin-1- |
| | 0300524, | yl)methyl)pyrido[3,2-d]pyrimidin-4-ylamino)-2,2′-dimethylbiphenyl-3- |
| | Example | yl)benzo[d]oxazol-5-yl)methyl)piperidine-4-carboxylic acid |
| | #30 | |
| | | 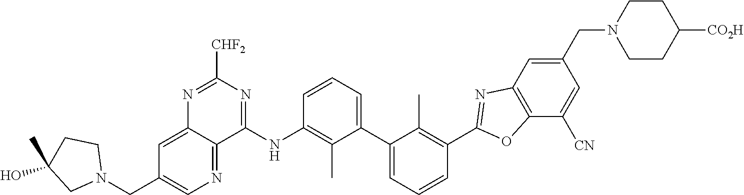 [SMILES: Cc1c(Nc2nc(C(F)F)nc3cc(CN4CC[C@@](C)(O)C4)cnc23)cccc1-c1cccc(-c2nc3cc(CN4CCC(C(=O)O)CC4)cc(C#N)c3o2)c1C] |
| 28 | US 2019- | (S)-1-((7-cyano-2-(3′-(2-(difluoromethyl)-7-((3-hydroxy-3-methylpyrrolidin-1- |
| | 0300524, | yl)methyl)pyrido[3,2-d]pyrimidin-4-ylamino)-2,2′-dimethylbiphenyl-3- |
| | Example | yl)benzo[d]oxazol-5-yl)methyl)piperidine-4-carboxylic acid |
| | #31 | |
| | | 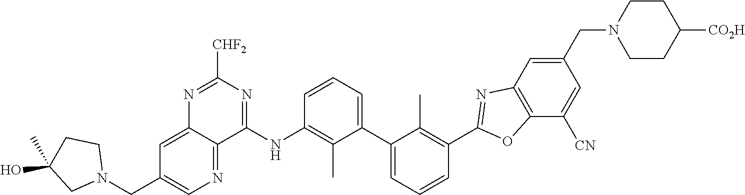 [SMILES: Cc1c(Nc2nc(C(F)F)nc3cc(CN4CC[C@](C)(O)C4)cnc23)cccc1-c1cccc(-c2nc3cc(CN4CCC(C(=O)O)CC4)cc(C#N)c3o2)c1C] |
| 29 | US 2019- | (R)-4-(2-(2-((2,2′-dichloro-3′-(5-(2-hydroxypropyl)-1-methyl-4,5,6,7-tetrahydro- |
| | 0345170, | 1H-imidazo[4,5-c]pyridine-2-carboxamido)-[1,1′-biphenyl]-3-yl)carbamoyl)-1- |
| | Example | methyl-1,4,6,7-tetrahydro-5H-imidazo[4,5-c]pyridin-5- |
| | #13 | yl)ethyl)bicyclo[2.2.1]heptane-1-carboxylic acid |
| | | 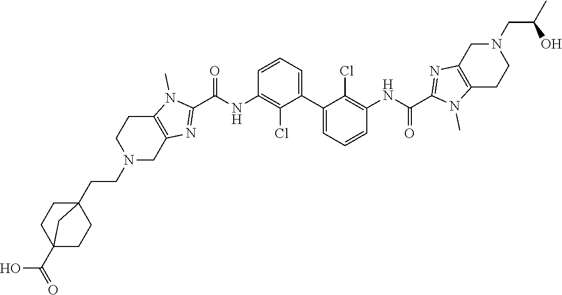 [SMILES: C[C@@H](O)CN1CCc2c(nc(C(=O)Nc3cccc(-c4cccc(NC(=O)c5nc6c(n5C)CCN(CCC57CCC(C(=O)O)(CC5)C7)C6)c4Cl)c3Cl)n2C)C1] |
| 30 | US 2019- | 4,4′-(((((2,2′-dichloro-[1,1′-biphenyl]-3,3′- |
| | 0345170, | diyl)bis(azanediyl))bis(carbonyl))bis(1-methyl-1,4,6,7-tetrahydro-5H- |
| | Example | imidazo[4,5-c]pyridine-2,5-diyl))bis(ethane-2,1-diyl))bis(bicyclo[2.2.1]heptane- |
| | #17 | 1-carboxylic acid) |
| | | 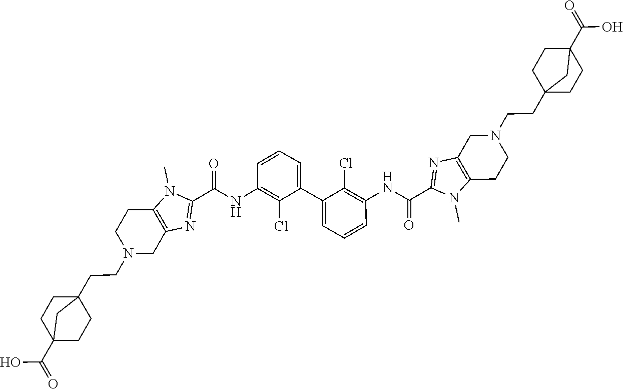 [SMILES: Cn1c(C(=O)Nc2cccc(-c3cccc(NC(=O)c4nc5c(n4C)CCN(CCC46CCC(C(=O)O)(CC4)C6)C5)c3Cl)c2Cl)nc2c1CCN(CCC13CCC(C(=O)O)(CC1)C3)C2] |
| 31 | US 2019- | 4-((2-((3′-(5-(2-(4-carboxybicyclo[2.2.1]heptan-1-yl)ethyl)-1-methyl-4,5,6,7- |
| | 0345170, | tetrahydro-1H-imidazo[4,5-c]pyridine-2-carboxamido)-2,2′-dichloro-[1,1′- |
| | Example | biphenyl]-3-yl)carbamoyl)-1-methyl-1,4,6,7-tetrahydro-5H-imidazo[4,5- |
| | #18 | c]pyridin-5-yl)methyl)bicyclo[2.2.1]heptane-1-carboxylic acid |
| | | 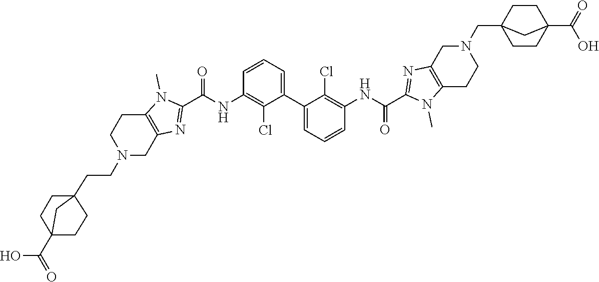 [SMILES: Cn1c(C(=O)Nc2cccc(-c3cccc(NC(=O)c4nc5c(n4C)CCN(CC46CCC(C(=O)O)(CC4)C6)C5)c3Cl)c2Cl)nc2c1CCN(CCC13CCC(C(=O)O)(CC1)C3)C2] |
| 32 | US 2019- | 4,4′-(((((2-chloro-2′-methyl-[1,1′-biphenyl]-3,3′- |
| | 0345170, | diyl)bis(azanediyl))bis(carbonyl))bis(1-methyl-1,4,6,7-tetrahydro-5H- |
| | Example | imidazo[4,5-c]pyridine-2,5-diyl))bis(ethane-2,1-diyl))bis(bicyclo[2.2.1]heptane- |
| | #34 | 1-carboxylic acid) |
| | | 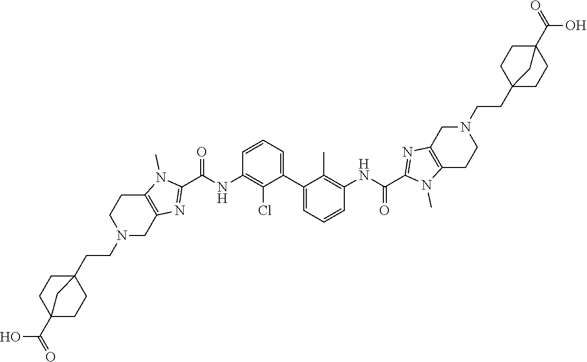 [SMILES: Cc1c(NC(=O)c2nc3c(n2C)CCN(CCC24CCC(C(=O)O)(CC2)C4)C3)cccc1-c1cccc(NC(=O)c2nc3c(n2C)CCN(CCC24CCC(C(=O)O)(CC2)C4)C3)c1Cl] |
| 33 | US 2019- | 4,4′-(((((2-chloro-2′-cyano-[1,1′-biphenyl]-3,3′- |
| | 0345170, | diyl)bis(azanediyl))bis(carbonyl))bis(1-methyl-1,4,6,7-tetrahydro-5H- |
| | Example | imidazo[4,5-c]pyridine-2,5-diyl))bis(ethane-2,1-diyl))bis(bicyclo[2.2.1]heptane- |
| | #51 | 1-carboxylic acid) |
| | | 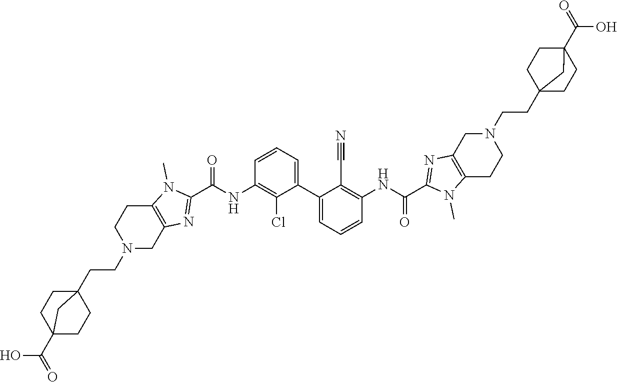 [SMILES: Cn1c(C(=O)Nc2cccc(-c3cccc(NC(=O)c4nc5c(n4C)CCN(CCC46CCC(C(=O)O)(CC4)C6)C5)c3C#N)c2Cl)nc2c1CCN(CCC13CCC(C(=O)O)(CC1)C3)C2] |
| 34 | US 2021- | (R)-4-(2-(2-((2-chloro-3′-((2-(difluoromethyl)-7-((3-hydroxypyrrolidin-1- |
| | 0094976, | yl)methyl)pyrido[3,2-d]pyrimidin-4-yl)amino)-2′-methyl-[1,1′-biphenyl]-3- |
| | Example | yl)carbamoyl)-1-methyl-1,4,6,7-tetrahydro-5H-imidazo[4,5-c]pyridin-5- |
| | #1 | yl)ethyl)bicyclo[2.2.1]heptane-1-carboxylic acid |
| | | 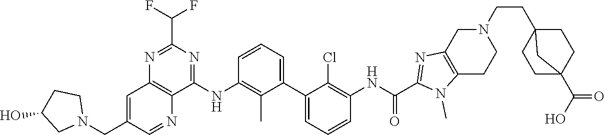 [SMILES: Cc1c(Nc2nc(C(F)F)nc3cc(CN4CC[C@@H](O)C4)cnc23)cccc1-c1cccc(NC(=O)c2nc3c(n2C)CCN(CCC24CCC(C(=O)O)(CC2)C4)C3)c1Cl] |

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012 (INCMGA0012; retifanlimab). In some embodiments, the anti-PD1 antibody is SHR-1210. Other anti-cancer agent(s) include antibody therapeutics such as 4-1 BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MED14736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MED14736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, US 2019/0300524, and US 2019/0345170, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31 M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of P13K-gamma. In some embodiments, the inhibitor of P13K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MED19447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1 BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MED11873, or MED16469. In some embodiments, the agonist of an immune checkpoint molecule is an agonist of OX40, e.g., OX40 agonist antibody or OX40L fusion protein. In some embodiments, the anti-OX40 antibody is INCAGN01949, MED10562 (tavolimab), MOXR-0916, PF-04518600, GSK3174998, BMS-986178, or 9B12. In some embodiments, the OX40L fusion protein is MED16383.

In some embodiments, the agonist of an immune checkpoint molecule is an agonist of CD40. In some embodiments, the agonist of CD40 is CP-870893, ADC-1013, CDX-1140, SEA-CD40, RO7009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MED19197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK 04.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound in a single or continuous dosage form, or they can be administered simultaneously or sequentially as separate dosage forms.

### IV. Formulation, Dosage Forms and Administration

When employed as pharmaceuticals, the compounds of the present disclosure can be administered in the form of pharmaceutical compositions. Thus, the present disclosure provides a composition comprising a compound of Formula (I), a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the present disclosure, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the present disclosure may be milled using known milling procedures such as wet milling to obtain a particle size appropriate for tablet formation and for other formulation types. Finely divided (nanoparticulate) preparations of the compounds of the present disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the present disclosure can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™)

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the present disclosure in a pharmaceutical composition can vary depending upon a number of factors including dosage, chemical characteristics (e.g., hydrophobicity), and the route of administration. For example, the compounds of the present disclosure can be provided in an aqueous physiological buffer solution containing about 0.1 to about 10% w/v of the compound for parenteral administration. Some typical dose ranges are from about 1 μg/kg to about 1 μg/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyloxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the present disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the present disclosure in a pharmaceutical composition can vary depending upon a number of factors including dosage, chemical characteristics (e.g., hydrophobicity), and the route of administration. For example, the compounds of the present disclosure can be provided in an aqueous physiological buffer solution containing about 0.1 to about 10% w/v of the compound for parenteral administration. Some typical dose ranges are from about 1 μg/kg to about 1 μg/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating KRAS protein in tissue samples, including human, and for identifying KRAS ligands by inhibition binding of a labeled compound. Substitution of one or more of the atoms of the compounds of the present disclosure can also be useful in generating differentiated ADME (Adsorption, Distribution, Metabolism and Excretion). Accordingly, the present disclosure includes KRAS binding assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). A “radio-labelled” compound can refer to an isotopically-labelled compound in which one or more atoms are replaced or substituted by an atom of an isotope that is radioactive.

Suitable isotopes that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶C, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups in Formula (I) can be perdeuterated. The symbol D included in a chemical formula or as a substituent indicates that deuterium is incorporated in the position labelled at greater than natural abundance, and typically indicates an abundance of equal to or greater than 50%, preferably equal to or greater than 90% or equal to or greater than 95%, 96%, 97%, 98%, 99%, 99.5%, 99.9%, 99.95%, or 99.99% relative to other forms of hydrogen.

Synthetic methods for including isotopes into organic compounds are known in the art (A. F. Thomas, Deuterium Labeling in Organic Chemistry, (Appleton-Century-Crofts, New York, N.Y., 1971); J. Atzrodt, et al., Angew. Chem. Int. Ed., 2007, 7744-65; J. R. Hanson, The Organic Chemistry of Isotopic Labelling, (Royal Society of Chemistry, 2011)). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes, et al., J. Med. Chem. 2011, 54(1), 201-10; R. Xu et al., J. Label. Compd. Radiopharm. 2015, 58, 308-12). In particular, substitution at one or more metabolism sites may afford one or more of the therapeutic advantages.

The radionuclide that is incorporated in the instant radio-labeled compounds will depend on the specific application of that radio-labeled compound. For example, for in vitro adenosine receptor labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the present disclosure can be used in a screening assay to identify and/or evaluate compounds. For example, a newly synthesized or identified compound (i.e., test compound) which is labeled can be evaluated for its ability to bind a KRAS protein by monitoring its concentration variation when contacting with the KRAS, through tracking of the labeling. For example, a test compound (labeled) can be evaluated for its ability to reduce binding of another compound which is known to bind to a KRAS protein (i.e., standard compound). Accordingly, the ability of a test compound to compete with the standard compound for binding to the KRAS protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### VI. Kits

The present disclosure also includes pharmaceutical kits useful, e.g., in the treatment or prevention of diseases or disorders associated with the activity of KRAS, such as cancer or infections, which include one or more containers containing a pharmaceutical composition comprising a therapeutically effective amount of a compound of Formula (I), or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The disclosure will be described in greater detail by way of specific examples. The following examples are offered for illustrative purposes, and are not intended to limit the disclosure in any manner. Those of skill in the art will readily recognize a variety of non-critical parameters which can be changed or modified to yield essentially the same results. The compounds of the Examples have been found to inhibit the activity of KRAS according to at least one assay described herein.

### EXAMPLES

Experimental procedures for compounds of the present disclosure are provided below. Preparatory LCMS purifications of some of the compounds prepared were performed on Waters mass directed fractionation systems. The basic equipment setup, protocols, and control software for the operation of these systems have been described in detail in the literature. See e.g., K. F. Blom, J. Combi. Chem., 2002, 4(4), 295-301; K. F. Blom, et al., J. Combi. Chem., 2003, 5(5), 670-683; and K. F. Blom, et al., J. Combi. Chem. 2004, 6(6), 874-83. The compounds separated were typically subjected to analytical liquid chromatography mass spectrometry (LCMS) for purity check.

The compounds separated were typically subjected to analytical LCMS for purity check under the following conditions: Instrument; Agilent 1100 series, LC/MSD, Column: Waters SUNFIRE® C₁₈ 5 μm particle size, 2.1×5.0 mm, Buffers: mobile phase A: 0.025% TFA in water and mobile phase B: MeCN; gradient 2% to 80% of B in 3 min. with flow rate 2.0 mL/min.

Some of the compounds prepared were also separated on a preparative scale by RP-HPLC with MS detector or FCC (silica gel) as indicated in the Examples. Typical prep. RP-HPLC column conditions are as follows:

pH=2 purifications: Waters SUNFIRE® C₁₈ 5 μm particle size, 19×100 mm column, eluting with mobile phase A: 0.1% TFA in water and mobile phase B: MeCN; the flow rate was 30 mL/min., the separating gradient was optimized for each compound using the Compound Specific Method Optimization protocol as described in the literature [see K. F. Blom, et al., J. Combi. Chem. 2004, 6(6), 874-83]. Typically, the flow rate used with the 30×100 mm column was 60 mL/min.

pH=10 purifications: Waters XBRIDGE® C₁₈ 5 μm particle size, 19×100 mm column, eluting with mobile phase A: 0.15% NH₄OH in water and mobile phase B: MeCN; the flow rate was 30 mL/min., the separating gradient was optimized for each compound using the Compound Specific Method Optimization protocol as described in the literature [See K. F. Blom, et al., J. Combi. Chem. 2004, 6(6), 874-83]. Typically, the flow rate used with 30×100 mm column was 60 mL/min.

The following abbreviations may be used herein: AcOH (acetic acid); Ac₂O (acetic anhydride); aq. (aqueous); atm. (atmosphere(s)); Boc (t-butoxycarbonyl); Boc₂O (di-t-butyl dicarbonate); BOP ((benzotriazol-1-yloxy)tris(dimethylamino)phosphonium hexafluorophosphate); B₂pin₂ (bis(pinacolato)diboron); br (broad); cataCXium® A Pd G3 ([(di(1-adamantyl)-butylphosphine)-2-(2′-amino-1,1′-biphenyl)]palladium(II) methanesulfonate); Cbz (carboxybenzyl); calc. (calculated); CHCl₃ (chloroform); CO (carbon monoxide); CO₂ (carbon dioxide); COD (cycloocta-1,5-diene); Cs₂CO₃ (cesium carbonate); CsF (cesium fluoride); d (doublet); dd (doublet of doublets); DBU (1,8-diazabicyclo[5.4.0]undec-7-ene); DCM (dichloromethane); DIAD (N,N′-diisopropyl azidodicarboxylate); DIPEA (N,N-diisopropylethylamine); DIBAL (diisobutylaluminium hydride); DMF (N,N-dimethylformamide); DMAP (4-dimethylaminopyridine); DMSO (dimethylsulfoxide); eq. (equivalent(s)); Et (ethyl); EtOH (ethanol); EtOAc (ethyl acetate); Ex. (Example); FCC (flash column chromatography); g (gram(s)); h (hour(s)); H₂ (hydrogen); HATU (N,N, N′,N′tetramethyl-O-(7-azabenzotriazol-1-yl)uronium hexafluorophosphate); HCl (hydrochloric acid or hydrogen chloride); HPLC (high performance liquid chromatography); Hz (hertz); [lr(OMe)(COD)]₂ ((1,5-cyclooctadiene)(methoxy)iridium(1) dimer); J (coupling constant); K₂CO₃ (potassium carbonate); KI (potassium iodide); KOtBu (potassium tert-butoxide); K₃PO₄ (tribasic potassium phosphate); LCMS (liquid chromatography-mass spectrometry); LiBH₄ (lithium borohydride); LDA (lithium diisopropylamide); LHMDS (lithium bis(trimethylsilyl)amide); m (multiplet); M (molar); mCPBA (3-chloroperoxybenzoic acid); MS (Mass spectrometry); Me (methyl); MeCN (acetonitrile); MeOH (methanol); mg (milligram(s)); MgSO₄ (magnesium sulfate); min. (minutes(s)); mL (milliliter(s)); mmol (millimole(s)); N (normal); N₂ (nitrogen); NaBH₄ (sodium borohydride); NaBH(OAc)₃ (sodium triacetoxyborohydride); Na₂CO₃ (sodium carbonate); NaCl (sodium chloride); NADPH (nicotinamide adenine dinucleotide phosphate); NaH (sodium hydride); NaHCO₃ (sodium bicarbonate); NaHMDS (sodium bis(trimethylsilyl)amide); NalO₄ (sodium metaperiodate); NaOH (sodium hydroxide); Na₂SO₄ (sodium sulfate); Na₂SO₃ (sodium sulfite); NaHCO₃ (sodium thiosulfate); NCS (N-chlorosuccinimide); NEt₃ (triethylamine); NH₄Cl (ammonium chloride); NH₄OH (ammonium hydroxide); nM (nanomolar); NMP (N-methylpyrrolidinone); NMR (nuclear magnetic resonance spectroscopy); OTf (trifluoromethanesulfonate); Pd(amphos)Cl₂ (bis(di-tert-butyl(4-dimethylaminophenyl)phosphine)dichloropalladium(II)); Ph (phenyl); Pd(PPh₃)₄(tetrakis(triphenylphosphine) palladium(0)); pM (picomolar); PPT(precipitate); prep. (preparative); RP-HPLC (reverse phase high performance liquid chromatography); r.t. (room temperature), RuPhos (2-dicyclohexylphosphino-2′,6′-diisopropoxybiphenyl); RuPhos Pd G4 ([Dicyclohexyl(2′,6′-diisopropoxy-2-biphenylyl)phosphine-κP](methanesulfonatato-κO)[2′-(methylamino-κN)-2-biphenylyl-κC²]palladium); s (singlet); sat. (saturated); Selectfluor (1-chloromethyl-4-fluoro-1,4-diazoniabicyclo[2.2.2]octane bis(tetrafluoroborate)); t (triplet or tertiary); TBAF (tetra-n-butylammonium fluoride); TBDPS (tert-butyldiphenylsilyl); TBS (tert-butyldimethylsilyl); TEMPO (2,2,6,6-tetramethylpiperidine-N-oxyl); tert (tertiary); tt (triplet of triplets); TFA (trifluoroacetic acid); THF (tetrahydrofuran); TMS-OTf (trimethylsilyl trifluoromethanesulfonate); p-TsOH (p-toluenesulfonic acid); μg (microgram(s)); μL (microliter(s)); μM (micromolar); wt % (weight percent); xantphos (4,5-bis(diphenylphosphino)-9,9-dimethylxanthene); XPhos Pd G4 ((SP-4-3)-[dicyclohexyl[2′,4′,6′-tris(1-methylethyl)[1,1′-biphenyl]-2-yl]phosphine](methanesulfonato-κO)[2′-(methylamino-κN)[1,1′-biphenyl]-2-yl-κC]palladium). Brine is sat. aq. NaCl. In vacuo is under vacuum.

### Intermediate 1. tert-Butyl (3-cyano-4-(3,5-dimethyl-1H-pyrazol-4-yl)-7-fluorobenzo[b]thiophene-2-yl)carbamate

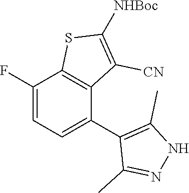 [SMILES: Cc1n[nH]c(C)c1-c1ccc(F)c2sc(NC(=O)OC(C)(C)C)c(C#N)c12]

A solution of tert-butyl (4-bromo-3-cyano-7-fluorobenzo[b]thiophene-2-yl)carbamate (500 mg, 1.347 mmol), tert-butyl 3,5-dimethyl-4-(4,4,5,5-tetramethyl-1,3,2-dioxaborolan-2-yl)-1H-pyrazole-1-carboxylate (651 mg, 2.020 mmol), PdCl₂(dppf)—CH₂Cl₂ adduct (220 mg, 0.269 mmol) and potassium carbonate (558 mg, 4.04 mmol) in 1,4-dioxane (10 mL) and water (2.5 mL) was stirred at 100° C. for 5 h. Upon completion, the mixture was diluted with EtOAc, washed with water and brine, dried over anhydrous Na₂SO₄, and concentrated in vacuo. The residue was purified by FCC (eluting with 0˜100% EtOAc in hexanes) to afford the title compound (400 mg, 77% yield). LC-MS calc. for C₁₉H₂₀FN₄O₂S (M+H)⁺: m/z=387.1; found 387.1.

### Intermediate 2. tert-Butyl (4-(1-(4-chloro-6-((S)-1-((S)-1-methylpyrrolidin-2-yl)ethoxy)pyrimidin-2-yl)-3,5-dimethyl-1H-pyrazol-4-yl)-3-cyano-7-fluorobenzo[b]thiophene-2-yl)carbamate

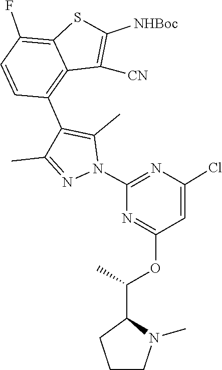 [SMILES: Cc1nn(-c2nc(Cl)cc(O[C@@H](C)[C@@H]3CCCN3C)n2)c(C)c1-c1ccc(F)c2sc(NC(=O)OC(C)(C)C)c(C#N)c12]

### Step 1. 4-Chloro-6-((S)-1-((S)-1-methylpyrrolidin-2-yl)ethoxy)-2-(methylsulfonyl)pyrimidine

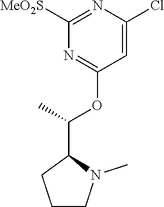 [SMILES: C[C@H](Oc1cc(Cl)nc(S(C)(=O)=O)n1)[C@@H]1CCCN1C]

DIPEA (7.85 mL, 44.9 mmol) was added to a solution of 4,6-dichloro-2-(methylsulfonyl) pyrimidine (5.1 g, 22.46 mmol) and (S)-1-((S)-1-methylpyrrolidin-2-yl)ethan-1-ol (3.19 g, 24.71 mmol) in MeCN (80 mL) at r.t., then the mixture was stirred at r.t. for 2 h. The solution was concentrated, then purified by FCC (0˜10% MeOH in DCM), to provide the title compound (3.2 g, 44% yield). LC-MS calc. for C₁₂H₁₉ClN₃O₃S (M+H)⁺: m/z=320.1; found 320.1.

### Step 2. tert-Butyl (4-(1-(4-chloro-6-((S)-1-((S)-1-methylpyrrolidin-2-yl)ethoxy)pyrimidin-2-yl)-3,5-dimethyl-1H-pyrazol-4-yl)-3-cyano-7-fluorobenzo[b]thiophene-2-yl)carbamate

Cs₂CO₃ (759 mg, 2.33 mmol) was added to a solution of Intermediate 1 (300 mg, 0.776 mmol) and 4-chloro-6-((S)-1-((S)-1-methylpyrrolidin-2-yl)ethoxy)-2-(methylsulfonyl)pyrimidine (497 mg, 1.553 mmol) in MeCN (10 mL) at r.t. After stirring at r.t. for 3 h, the insoluble salts were filtered off, and the filtrate was concentrated. The residue was purified by FCC (0˜100% EtOAc in hexanes) to afford the title compound (365 mg, 75% yield). LC-MS calc. for C₃₀H₃₄ClFN₇O₃S (M+H)⁺: m/z=626.2; found 626.2.

Alternative stereoisomers of the Intermediate 2 can be prepared by an analogous method using an alternative stereoisomer of 1-(1-methylpyrrolidin-2-yl)ethan-1-ol.

### Intermediate 3. tert-Butyl (4-(1-(6-chloro-2-((S)-1-((S)-1-methylpyrrolidin-2-yl)ethoxy)pyrimidin-4-yl)-3,5-dimethyl-1H-pyrazol-4-yl)-3-cyano-7-fluorobenzo[b]thiophene-2-yl)carbamate

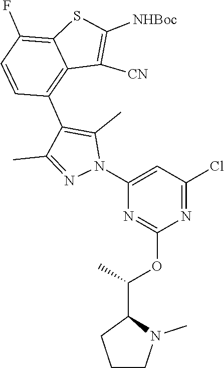 [SMILES: Cc1nn(-c2cc(Cl)nc(O[C@@H](C)[C@@H]3CCCN3C)n2)c(C)c1-c1ccc(F)c2sc(NC(=O)OC(C)(C)C)c(C#N)c12]

### Step 1. tert-Butyl (4-(1-(6-chloro-2-(methylthio)pyrimidin-4-yl)-3,5-dimethyl-1H-pyrazol-4-yl)-3-cyano-7-fluorobenzo[b]thiophene-2-yl)carbamate

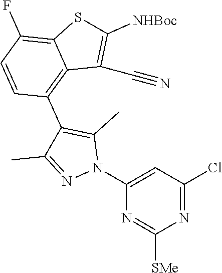 [SMILES: CSc1nc(Cl)cc(-n2nc(C)c(-c3ccc(F)c4sc(NC(=O)OC(C)(C)C)c(C#N)c34)c2C)n1]

Cs₂CO₃ (1265 mg, 3.88 mmol) was added to a solution of Intermediate 1 (500 mg, 1.29 mmol) and 4,6-dichloro-2-(methylthio)pyrimidine (757 mg, 3.88 mmol) in MeCN (12 mL), then the mixture was stirred at 90° C. for 1 h. After cooling to r.t., the insoluble salts were filtered off, and the filtrate was concentrated. The residue was purified by FCC (0˜40% EtOAc in hexanes) to afford the title compound (630 mg, 89% yield). LC-MS calc. for C₂₄H₂₃ClFN₆O₂S₂ (M+H)⁺: m/z=545.1; found 545.1.

### Step 2. tert-Butyl (4-(1-(6-chloro-2-(methylsulfonyl)pyrimidin-4-yl)-3,5-dimethyl-1H-pyrazol-4-yl)-3-cyano-7-fluorobenzo[b]thiophene-2-yl)carbamate

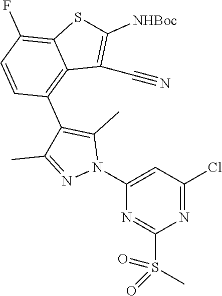 [SMILES: Cc1nn(-c2cc(Cl)nc(S(C)(=O)=O)n2)c(C)c1-c1ccc(F)c2sc(NC(=O)OC(C)(C)C)c(C#N)c12]

mCPBA (841 mg, 4.88 mmol) was added to a solution of tert-butyl (4-(1-(6-chloro-2-(methylthio)pyrimidin-4-yl)-3,5-dimethyl-1H-pyrazol-4-yl)-3-cyano-7-fluorobenzo[b]thiophen-2-yl)carbamate (1.06 g, 1.95 mmol) in DCM (10 mL) at 0° C. After stirring at 0° C. for 10 min., the mixture was allowed to warm to r.t., and continue stirring at r.t. with LCMS monitoring. Upon completion, the reaction was quenched with aq. NaHCO₃ and aq. Na₂S₂O₃, then the mixture was extracted with DCM (3×). The organic phase was dried over Na₂SO₄, then concentrated under reduced pressure. The residue was purified by FCC (0-100% EtOAc in hexanes) to give the title compound (1.08 g, 96% yield). LC-MS calc. for C₂₄H₂₃ClFN₆O₄S₂ (M+H)⁺: m/z=577.1; found 577.1.

### Step 3. tert-Butyl (4-(1-(6-chloro-2-((S)-1-((S)-1-methylpyrrolidin-2-yl)ethoxy)pyrimidin-4-yl)-3,5-dimethyl-1H-pyrazol-4-yl)-3-cyano-7-fluorobenzo[b]thiophene-2-yl)carbamate

The product from the previous step (1.08 g, 1.87 mmol) was dissolved in THF (10 mL), then (S)-1-((S)-1-methylpyrrolidin-2-yl)ethan-1-ol (0.53 g, 4.11 mmol) was added. The solution was cooled to 0° C., and LHMDS (1 M in THF, 4.11 mL, 4.11 mmol) was added dropwise. Then the mixture was stirred at 0° C. for 1 h with LCMS monitoring. Upon completion, the reaction was quenched with sat. aq. NH₄Cl, and extracted with EtOAc. The organic phase was washed with brine, dried over anhydrous Na₂SO₄, filtered and concentrated. The residue was purified by FCC (0˜100% EtOAc in hexanes) to afford the title compound (0.93 g, 79% yield). LC-MS calc. for C₃₀H₃₄ClFN₇O₃S (M+H)⁺: m/z=626.2; found 626.2.

Alternative stereoisomers of the Intermediate 3 can be prepared by an analogous method using an alternative stereoisomer of 1-(1-methylpyrrolidin-2-yl)ethan-1-ol.

### Intermediate 4. tert-Butyl (7-fluoro-4-(4,4,5,5-tetramethyl-1,3,2-dioxaborolan-2-yl)benzo[b]thiophene-2-yl)carbamate

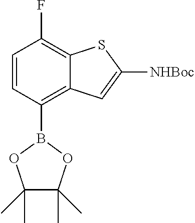 [SMILES: CC(C)(C)OC(=O)Nc1cc2c(B3OC(C)(C)C(C)(C)O3)ccc(F)c2s1]

A mixture of tert-butyl (4-bromo-7-fluorobenzo[b]thiophene-2-yl)carbamate (3 g, 8.67 mmol), bis(pinacolato)diboron (3.96 g, 15.60 mmol), potassium acetate (2.55 g, 26.0 mmol) and dichloro[1,1′-bis(diphenylphosphino)ferrocene]palladium (II) dichloromethane adduct (0.708 g, 0.867 mmol), in 1,4-dioxane (60 mL) was stirred at 100° C. for 2 h. The mixture was then cooled to r.t., diluted with EtOAc, and washed with water and brine consecutively. The organic phase was dried over Na₂SO₄ and concentrated under reduced pressure. The residue was purified by FCC (eluting with 0˜20% EtOAc in hexanes) to afford the title compound as pale yellow solid (2.73 g, 80% yield). LC-MS calc.for C₁₉H₂₆BFNO₄S (M+H)⁺: m/z=394.2; found 394.2.

### Intermediate 5. 4-Chloro-6-(1-methyl-1H-pyrazol-4-yl)-2-(methylthio)pyrimidine

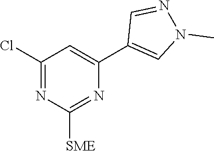

A mixture of 4,6-dichloro-2-(methylthio)pyrimidine (5.2 g, 26.7 mmol), 1-methyl-4-(4,4,5,5-tetramethyl-1,3,2-dioxaborolan-2-yl)-1H-pyrazole (5.55 g, 26.7 mmol), Pd(PPh₃)₂Cl₂ (1.87 g, 2.67 mmol) and Na₂CO₃ (8.48 g, 80 mmol) in THF (80 mL) and water (20 mL) was stirred at 70° C. for 5 h. Upon completion, the mixture was cooled to r.t., diluted with EtOAc, washed with water and brine, and dried over Na₂SO₄. The residue after concentration was purified by FCC (eluting with 0˜20% EtOAc in hexanes) to afford the title compound as white solid (5 g, 78% yield). LC-MS calc. for C₉H₁₀ClN4S (M+H)⁺: m/z=241.0; found 241.0.

### Intermediate 6a and 6b. tert-Butyl (3-cyano-7-fluoro-4-(5-methyl-1-(6-(1-methyl-1H-pyrazol-4-yl)-2-(methylthio)pyrimidin-4-yl)-3-vinyl-1H-pyrazol-4-yl)benzo[b]thiophene-2-yl)carbamate and tert-butyl (3-cyano-7-fluoro-4-(3-methyl-1-(6-(1-methyl-1H-pyrazol-4-yl)-2-(methylthio)pyrimidin-4-yl)-5-vinyl-1H-pyrazol-4-yl)benzo[b]thiophene-2-yl)carbamate

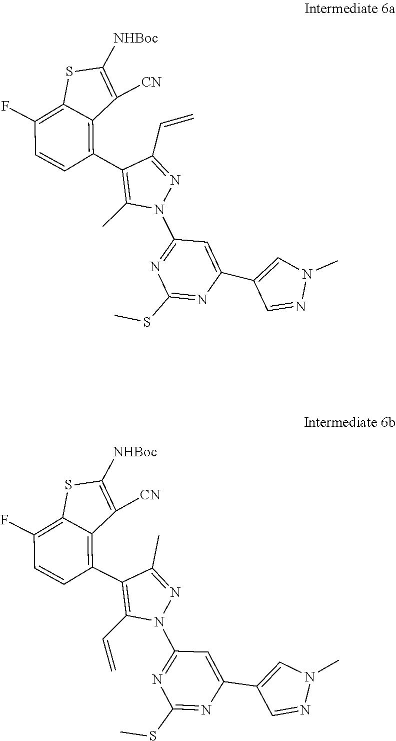 [SMILES: C=Cc1nn(-c2cc(-c3cnn(C)c3)nc(SC)n2)c(C)c1-c1ccc(F)c2sc(NC(=O)OC(C)(C)C)c(C#N)c12 ; C=Cc1c(-c2ccc(F)c3sc(NC(=O)OC(C)(C)C)c(C#N)c23)c(C)nn1-c1cc(-c2cnn(C)c2)nc(SC)n1]

### Step 1. 4-Bromo-3-methyl-5-vinyl-1H-pyrazole

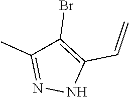

To a solution of methyltriphenylphosphonium bromide (19.8 g, 55.6 mmol) in THF (100 mL) was added a solution of potassium tert-butoxide (1 M in THF, 55.6 mL, 55.6 mmol), and the resulting mixture was stirred at r.t. for 1 h. Then 4-bromo-3-methyl-1H-pyrazole-5-carbaldehyde (3.0 g, 15.87 mmol) was added as a solid in one portion. The mixture was stirred at r.t. for 1 h with LC-MS monitoring. Upon completion, the mixture was quenched with water and diluted with EtOAc. The layers were separated and the aqueous layer was extracted with EtOAc. The combined organic fractions were dried over Na₂SO₄ and concentrated under reduced pressure. The residue was purified by FCC (0˜100% EtOAc in DCM) to afford the title compound (2.4 g, 81% yield). LC-MS calc. for C₆H₈BrN₂ (M+H)⁺: m/z=187.0; found 187.0.

### Step 2. 4-(4-Bromo-5-methyl-3-vinyl-1H-pyrazol-1-yl)-6-(1-methyl-1H-pyrazol-4-yl)-2-(methylthio)pyrimidine and 4-(4-bromo-3-methyl-5-vinyl-1H-pyrazol-1-yl)-6-(1-methyl-1H-pyrazol-4-yl)-2-(methylthio)pyrimidine

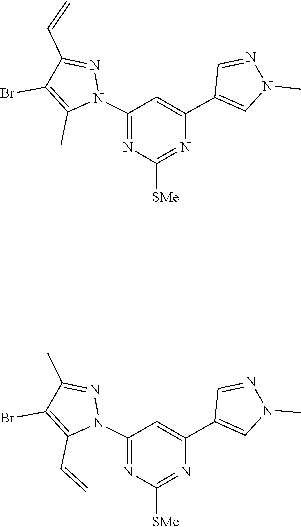 [SMILES: C=Cc1nn(-c2cc(-c3cnn(C)c3)nc(SC)n2)c(C)c1Br ; C=Cc1c(Br)c(C)nn1-c1cc(-c2cnn(C)c2)nc(SC)n1]

Cs₂CO₃ (8.36 g, 25.7 mmol) was added to a solution of 4-bromo-3-methyl-5-vinyl-1H-pyrazole (2.4 g, 12.83 mmol) and Intermediate 5 (3.71 g, 15.4 mmol) in MeCN (128 mL). The resulting mixture was stirred at 80° C. for 5 h. The mixture was cooled to r.t., then quenched with brine and diluted with EtOAc. The layers were separated, and the aqueous layer was extracted with EtOAc. The combined organic fractions were dried over Na₂SO₄ and concentrated under reduced pressure. The residue was purified by FCC (eluting with 0-100% EtOAc in hexanes) to afford the title compound as an inseperable mixture of two regioisomers (2.76 g, 55% yield). LC-MS calc. for C₁₅H₁₆BrN₆S (M+H)⁺: m/z=391.0; found 391.1.

### Step 3. tert-butyl (3-cyano-7-fluoro-4-(3-methyl-1-(6-(1-methyl-1H-pyrazol-4-yl)-2-(methylthio)pyrimidin-4-yl)-5-vinyl-1H-pyrazol-4-yl)benzo[b]thiophene-2-yl)carbamate and tert-butyl (3-cyano-7-fluoro-4-(5-methyl-1-(6-(1-methyl-1H-pyrazol-4-yl)-2-(methylthio)pyrimidin-4-yl)-3-vinyl-1H-pyrazol-4-yl)benzo[b]thiophene-2-yl)carbamate

A mixture of the product from Step 2 (500 mg, 1.28 mmol), tert-butyl (3-cyano-4-(5,5-dimethyl-1,3,2-dioxaborinan-2-yl)-7-fluorobenzo[b]thiophene-2-yl)carbamate (775 mg, 1.92 mmol), dichloro[bis(2-(diphenylphosphino)phenyl)ether]palladium(II) (91.0 mg, 0.128 mmol), and Cs₂CO₃ (2.08 g, 6.39 mmol) in DMSO (12.8 mL) was stirred under N₂ atmosphere at 100° C. for 1 h. The mixture was cooled to r.t., diluted with EtOAc, washed with water (3×) and brine, dried over Na₂SO₄ and concentrated. The residue was purified by FCC (eluting with 0-100% EtOAc in hexanes) to afford intermediate 6a (380 mg, 49% yield) which eluted out first and 6b (80 mg, 10% yield) which eluted out second.

LC-MS calc. for intermediate 6a C₂₉H₂₈FN₈O₂S₂ (M+H)⁺: m/z=603.2; found 603.2

LC-MS calc. for intermediate 6b C₂₉H₂₈FN₈O₂S₂ (M+H)⁺: m/z=603.2; found 603.2.

Alternative stereoisomers of the compounds below can be prepared by an analogous method using an alternative stereoisomer of Intermediate 2 or Intermediate 3.

### Intermediate 7. tert-Butyl (3-cyano-4-(1-(6-(4-(dimethylcarbamoyl)piperazin-1-yl)-2-(methylsulfonyl)pyrimidin-4-yl)-3,5-dimethyl-1H-pyrazol-4-yl)-7-fluorobenzo[b]thiophene-2-yl)carbamate

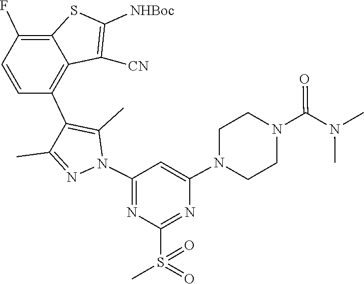 [SMILES: Cc1nn(-c2cc(N3CCN(C(=O)N(C)C)CC3)nc(S(C)(=O)=O)n2)c(C)c1-c1ccc(F)c2sc(NC(=O)OC(C)(C)C)c(C#N)c12]

### Step 1. tert-Butyl (3-cyano-4-(1-(6-(4-(dimethylcarbamoyl)piperazin-1-yl)-2-(methylthio)pyrimidin-4-yl)-3,5-dimethyl-1H-pyrazol-4-yl)-7-fluorobenzo[b]thiophene-2-yl)carbamate

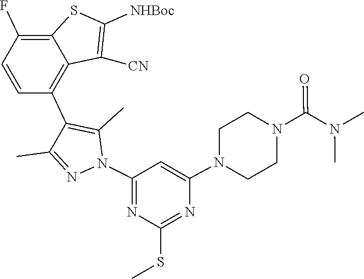 [SMILES: CSc1nc(N2CCN(C(=O)N(C)C)CC2)cc(-n2nc(C)c(-c3ccc(F)c4sc(NC(=O)OC(C)(C)C)c(C#N)c34)c2C)n1]

A mixture of tert-butyl (4-(1-(6-chloro-2-(methylthio)pyrimidin-4-yl)-3,5-dimethyl-1H-pyrazol-4-yl)-3-cyano-7-fluorobenzo[b]thiophene-2-yl)carbamate (Intermediate 3, Step 1, 600 mg, 1.101 mmol), N,N-dimethylpiperazine-1-carboxamide (208 mg, 1.321 mmol), and Cs₂CO₃ (1.1 g, 3.30 mmol) was heated at 90° C. for 2 h. The mixture was cooled to r.t., filtered, concentrated and purified by FCC (2-20% MeOH in DCM) to afford the title compound (290 mg, 40% yield). LC-MS calc. for C₃₁H₃₇FN₉O₃S₂ (M+H)⁺: m/z=666.2; found 666.2.

### Step 2. tert-Butyl (3-cyano-4-(1-(6-(4-(dimethylcarbamoyl)piperazin-1-yl)-2-(methylsulfonyl)pyrimidin-4-yl)-3,5-dimethyl-1H-pyrazol-4-yl)-7-fluorobenzo[b]thiophene-2-yl)carbamate

To a stirred solution of tert-butyl (3-cyano-4-(1-(6-(4-(dimethylcarbamoyl)piperazin-1-yl)-2-(methylthio)pyrimidin-4-yl)-3,5-dimethyl-1H-pyrazol-4-yl)-7-fluorobenzo[b]thiophene-2-yl)carbamate (500 mg, 0.751 mmol) in DCM (29 mL) at 0° C. was added a solution of mCPBA (272 mg, 1.577 mmol) in DCM (5 mL) dropwise. The mixture was stirred at r.t. for 2 h. The mixture was concentrated, loaded directly onto silica gel and purified by FCC (2-20% MeOH in DCM) to obtain the title compound (285 mg, 54% yield) along with a minor amout of the corresponding sulfoxide. LC-MS calc. for C₃₁H₃₇FN₉O₅S₂ (M+H)⁺: m/z=698.2; found 698.2.

### Intermediate 8. tert-Butyl (3-cyano-7-fluoro-4-(1-(6-(3-fluoro-4-(methylcarbamoyl)phenyl)-2-(methylsulfonyl)pyrimidin-4-yl)-3,5-dimethyl-1H-pyrazol-4-yl)benzo[b]thiophene-2-yl)carbamate

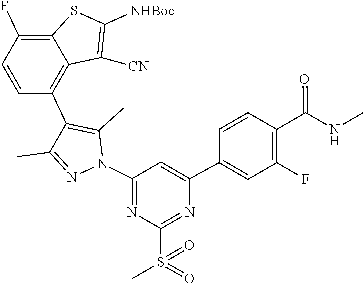 [SMILES: CNC(=O)c1ccc(-c2cc(-n3nc(C)c(-c4ccc(F)c5sc(NC(=O)OC(C)(C)C)c(C#N)c45)c3C)nc(S(C)(=O)=O)n2)cc1F]

### Step 1. tert-Butyl (3-cyano-7-fluoro-4-(1-(6-(3-fluoro-4-(methylcarbamoyl)phenyl)-2-(methylthio)pyrimidin-4-yl)-3,5-dimethyl-1H-pyrazol-4-yl)benzo[b]thiophene-2-yl)carbamate

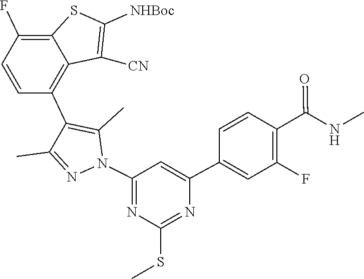 [SMILES: CNC(=O)c1ccc(-c2cc(-n3nc(C)c(-c4ccc(F)c5sc(NC(=O)OC(C)(C)C)c(C#N)c45)c3C)nc(SC)n2)cc1F]

To a mixture of tert-butyl (4-(1-(6-chloro-2-(methylthio)pyrimidin-4-yl)-3,5-dimethyl-1H-pyrazol-4-yl)-3-cyano-7-fluorobenzo[b]thiophene-2-yl)carbamate (Intermediate 3, Step 1, 1.4 g, 2.57 mmol), (3-fluoro-4-(methylcarbamoyl)phenyl)boronic acid (1.01 g, 5.14 mmol), Cs₂CO₃ (2.5 g, 7.7 mmol) and XPhos Pd G2 (200 mg, 0.26 mmol) were added 1,4-dioxane (8 mL) and water (2 mL). The reaction flask was placed under an atmosphere of N₂, then the mixture was stirred at 90° C. for 1 h. The mixture was diluted with water and extracted with EtOAc. The organic layer was washed with brine, dried over Na₂SO₄ and concentrated. The crude product was purified by FCC (10-100% EtOAc in hexanes) to provide the title compound (1.25 g, 73% yield). LC-MS calc. for C₃₂H₃₀F₂N₇O₃S₂ (M+H)⁺: m/z=662.2; found 662.2.

### Step 2. tert-Butyl (3-cyano-7-fluoro-4-(1-(6-(3-fluoro-4-(methylcarbamoyl)phenyl)-2-(methylsulfonyl)pyrimidin-4-yl)-3,5-dimethyl-1H-pyrazol-4-yl)benzo[b]thiophene-2-yl)carbamate

To a solution of the product from the previous step (1.25 g, 1.89 mmol) in DCM (18 mL) was added mCPBA (720 mg, 4.16 mmol) and the mixture was stirred at r.t. for 2 h. The mixture was then concentrated and purified directly by FCC (1-15% MeOH in DCM) to provide the title compound. LC-MS calc. for C₃₂H₃₀F₂N₇O₅S₂ (M+H)⁺: m/z=694.2; found 694.2

### Intermediate 9. tert-Butyl (3-cyano-4-(5-ethyl-3-methyl-1H-pyrazol-4-yl)-7-fluorothieno[3,2-c]pyridin-2-yl)carbamate

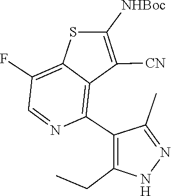 [SMILES: CCc1[nH]nc(C)c1-c1ncc(F)c2sc(NC(=O)OC(C)(C)C)c(C#N)c12]

### Step 1. tert-Butyl 4-bromo-5-ethyl-3-methyl-1H-pyrazole-1-carboxylate

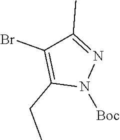 [SMILES: CCc1c(Br)c(C)nn1C(=O)OC(C)(C)C]

To a solution of 4-bromo-3-ethyl-5-methyl-1H-pyrazole (25 g, 132 mmol) in THF (882 mL) in a round-bottom flask were added boc anhydride (36.8 mL, 159 mmol), triethylamine (37 mL, 264 mmol) and DMAP (0.81 g, 6.61 mmol). The resulting mixture was stirred at r.t. for 1 h, then concentrated under reduced pressure. The crude product was purified by FCC (0% to 20% EtOAc in hexanes) to provide the title compound (33.1 g, 87% yield). LC-MS calc. for C₁₁H₁₈BrN₂O₂(M+H)⁺: m/z=289.0, 291.0; found 289.0, 291.0.

### Step 2. tert-Butyl 5-ethyl-3-methyl-4-(4,4,5,5-tetramethyl-1,3,2-dioxaborolan-2-yl)-1H-pyrazole-1-carboxylate

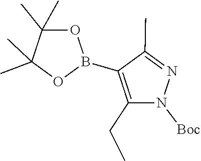 [SMILES: CCc1c(B2OC(C)(C)C(C)(C)O2)c(C)nn1C(=O)OC(C)(C)C]

To a mixture of tert-butyl 4-bromo-3-ethyl-5-methyl-1H-pyrazole-1-carboxylate (33.1 g, 114 mmol) and 2-Isopropoxy-4,4,5,5-tetramethyl-1,3,2-dioxaborolane (117 mL, 572 mmol) in THF (382 ml) at −78° C. was added dropwise n-butyllithium (2.5 M in hexanes, 183 mL, 458 mmol). The mixture was stirred at −78° C. for 1 h. The reaction was quenched at −78° C. with sat. aq. NH₄Cl solution, then filtered through diatomaceous earth. The filtrate was partitioned between water and EtOAc and the layers were separated. The organic layer was dried over Na₂SO₄ and concentrated. The crude product was purified by FCC (0-30% EtOAc in a 1/1 hexanes/DCM mixture) to provide the title compound (31.2 g, 81% yield). LC-MS calc. for C₁₇H₃₀BN₂O₄(M+H)⁺: m/z=337.2; found 337.2.

### Step 3. tert-butyl (3-cyano-4-(5-ethyl-3-methyl-1H-pyrazol-4-yl)-7-fluorothieno[3,2-c]pyridin-2-yl)carbamate

To a solution of tert-butyl (4-chloro-3-cyano-7-fluorothieno[3,2-c]pyridin-2-yl)carbamate (6.7 g, 20.44 mmol), tert-butyl 5-ethyl-3-methyl-4-(4,4,5,5-tetramethyl-1,3,2-dioxaborolan-2-yl)-1H-pyrazole-1-carboxylate (8.25 g, 24.53 mmol) in 1,4-dioxane (160 mL) and water (40 mL) were added RuPhos Pd G4 (3.48 g, 4.09 mmol), RuPhos (1.908 g, 4.09 mmol) and tripotassium phosphate (13.02 g, 61.3 mmol). The reaction flask was put under an atmosphere of N₂, then heated to 100° C. and stirred for 2 h. After compltion of the coupling reaction, additional water (30 mL) and potassium phosphate (13 g, 61.3 mmol) were added. Stirring was continued for an additional 2 h at 100° C. to afford full Boc deprotection. The mixture was extracted with EtOAc (3×). The organic layer was dried over magnesium suflate and concentrated. The crude product was purified by FCC (0-25% MeOH in DCM) to provide the title compound (5.5 g, 67% yield). LC-MS calc. for C₁₉H₂₁FN₅O₂S (M+H)⁺: m/z=402.1; found 402.1.

### Intermediate 10. tert-Butyl (4-(1-(6-chloro-2-(methylthio)pyrimidin-4-yl)-5-ethyl-3-methyl-1H-pyrazol-4-yl)-3-cyano-7-fluorothieno[3,2-c]pyridin-2-yl)carbamate

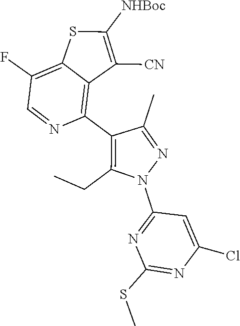 [SMILES: CCc1c(-c2ncc(F)c3sc(NC(=O)OC(C)(C)C)c(C#N)c23)c(C)nn1-c1cc(Cl)nc(SC)n1]

To a solution of Intermediate 9 (2.5 g, 6.23 mmol) in MeCN (62 mL) was added 4,6-dichloro-2-(methylthio)pyrimidine (2.429 g, 12.45 mmol) and Cs₂CO₃ (4.06 g, 12.45 mmol). The mixture was stirred at 90° C. for 3 h to provide a mixture of regioisomeric products. The mixture was cooled to r.t. and filtered over a pad of diatomaceous earth. The filtrate were concentrated and purified by FCC (0-50% EtOAc in 1/1 hexanes/DCM) to provide the title compound as a single regioisomer (1.0 g, 29% yield). LC-MS calc. for C₂₄H₂₄ClFN₇O₂S₂ (M+H)⁺: m/z=560.1; found 560.1.

### Example 1: 4-(1-(4-(4-Acetylpiperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile

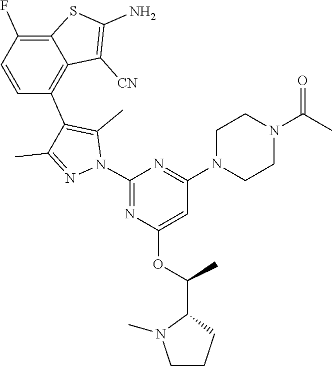 [SMILES: CC(=O)N1CCN(c2cc(O[C@@H](C)[C@@H]3CCCN3C)nc(-n3nc(C)c(-c4ccc(F)c5sc(N)c(C#N)c45)c3C)n2)CC1]

A mixture of Intermediate 2 (10 mg, 0.016 mmol), 1-(piperazin-1-yl)ethan-1-one (6.14 mg, 0.048 mmol), RuPhos Pd G4 (2.72 mg, 3.19 μmol), RuPhos (2.98 mg, 6.39 μmol) and Cs₂CO₃ (15.61 mg, 0.048 mmol) in 1,4-dioxane (0.5 mL) was bubbled with N₂ gas for 5 min. Then the mixture was stirred at 90° C. for 2 h. After cooling to r.t., the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc, for C₃₁H₃₇FN₉O₂S(M+H)⁺: m/z=618.3; found 618.3. * This peak is the more potent one.

Peak 2, diastereomer 2. LC-MS calc, for C₃₁H₃₇FN₉O₂S (M+H)⁺: m/z=618.3; found 618.3.

Diastereomers of the title compound are understood to be:
(Rₐ)-4(1-(4-(4-acetylpiperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile; and
(Sₐ)-4-(1-(4-(4-acetylpiperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile.

### Examples 2-17

The following compounds listed in Table 4 were prepared in an analogous fashion to Example 1 using the appropriate coupling partner.

TABLE 4
Examples 2-17

| Ex | | | ESI |
|---|---|---|---|
| # | Structure | IUPAC name | [M + H]⁺ |
| 2 | 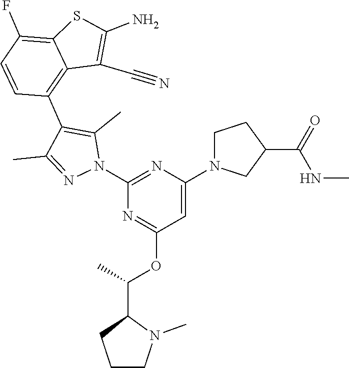 [SMILES: CNC(=O)C1CCN(c2cc(O[C@@H](C)[C@@H]3CCCN3C)nc(-n3nc(C)c(-c4ccc(F)c5sc(N)c(C#N)c45)c3C)n2)C1] | 1-(2-(4-(2-Amino-3-cyano-7- fluorobenzo[b]thiophen-4-yl)-3,5-dimethyl-1H- pyrazol-1-yl)-6-((S)-1-((S)-1-methylpyrrolidin-2- yl)ethoxy)pyrimidin-4-yl)-N-methylpyrrolidine-3- carboxamide | 618.3 |
| 3 | 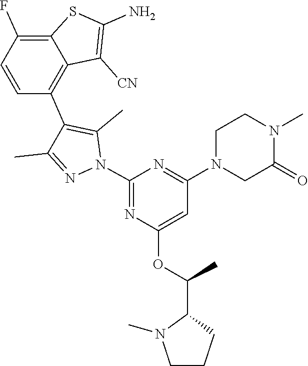 [SMILES: Cc1nn(-c2nc(O[C@@H](C)[C@@H]3CCCN3C)cc(N3CCN(C)C(=O)C3)n2)c(C)c1-c1ccc(F)c2sc(N)c(C#N)c12] | 2-Amino-4-(3,5-dimethyl-1-(4-(4-methyl-3- oxopiperazin-1-yl)-6-((S)-1-((S)-1- methylpyrrolidin-2-yl)ethoxy)pyrimidin-2-yl)-1H- pyrazol-4-yl)-7-fluorobenzo[b]thiophene-3- carbonitrile | 604.3 |
| 4 | 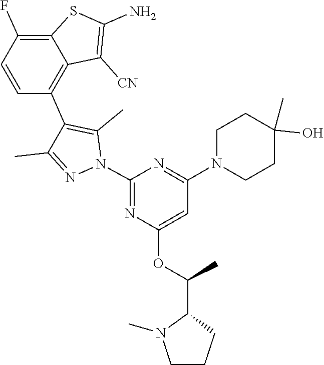 [SMILES: Cc1nn(-c2nc(O[C@@H](C)[C@@H]3CCCN3C)cc(N3CCC(C)(O)CC3)n2)c(C)c1-c1ccc(F)c2sc(N)c(C#N)c12] | 2-Amino-7-fluoro-4-(1-(4-(4-hydroxy-4- methylpiperidin-1-yl)-6-((S)-1-((S)-1- methylpyrrolidin-2-yl)ethoxy)pyrimidin-2-yl)-3,5- dimethyl-1H-pyrazol-4-yl)benzo[b]thiophene-3- carbonitrile | 605.3 |
| 5 | 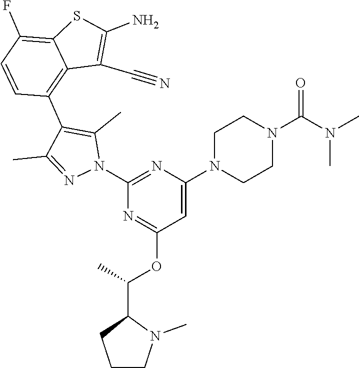 [SMILES: Cc1nn(-c2nc(O[C@@H](C)[C@@H]3CCCN3C)cc(N3CCN(C(=O)N(C)C)CC3)n2)c(C)c1-c1ccc(F)c2sc(N)c(C#N)c12] | 4-(2-(4-(2-Amino-3-cyano-7- fluorobenzo[b]thiophen-4-yl)-3,5-dimethyl-1H- pyrazol-1-yl)-6-((S)-1-((S)-1-methylpyrrolidin-2- yl)ethoxy)pyrimidin-4-yl)-N,N- dimethylpiperazine-1-carboxamide | 647.3 |
| 6 | 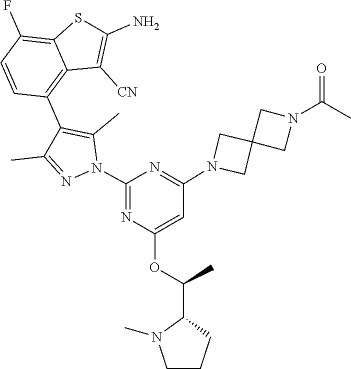 [SMILES: CC(=O)N1CC2(C1)CN(c1cc(O[C@@H](C)[C@@H]3CCCN3C)nc(-n3nc(C)c(-c4ccc(F)c5sc(N)c(C#N)c45)c3C)n1)C2] | 4-(1-(4-(6-Acetyl-2,6-diazaspiro[3.3]heptan-2- yl)-6-((S)-1-((S)-1-methylpyrrolidin-2- yl)ethoxy)pyrimidin-2-yl)-3,5-dimethyl-1H- pyrazol-4-yl)-2-amino-7- fluorobenzo[b]thiophene-3-carbonitrile | 630.3 |
| 7 | 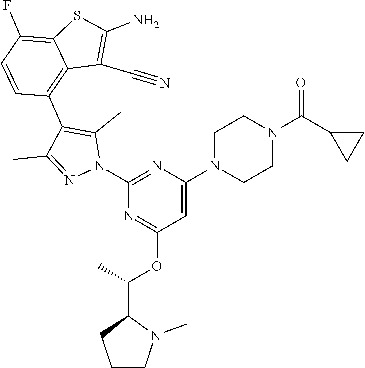 [SMILES: Cc1nn(-c2nc(O[C@@H](C)[C@@H]3CCCN3C)cc(N3CCN(C(=O)C4CC4)CC3)n2)c(C)c1-c1ccc(F)c2sc(N)c(C#N)c12] | 2-Amino-4-(1-(4-(4- (cyclopropanecarbonyl)piperazin-1-yl)-6-((S)-1- ((S)-1-methylpyrrolidin-2-yl)ethoxy)pyrimidin-2- yl)-3,5-dimethyl-1H-pyrazol-4-yl)-7- fluorobenzo[b]thiophene-3-carbonitrile | 644.3 |
| 8 | 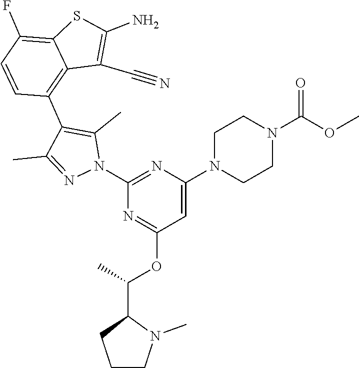 [SMILES: COC(=O)N1CCN(c2cc(O[C@@H](C)[C@@H]3CCCN3C)nc(-n3nc(C)c(-c4ccc(F)c5sc(N)c(C#N)c45)c3C)n2)CC1] | Methyl 4-(2-(4-(2-amino-3-cyano-7- fluorobenzo[b]thiophen-4-yl)-3,5-dimethyl-1H- pyrazol-1-yl)-6-((S)-1-((S)-1-methylpyrrolidin-2- yl)ethoxy)pyrimidin-4-yl)piperazine-1- carboxylate | 634.3 |
| 9 | 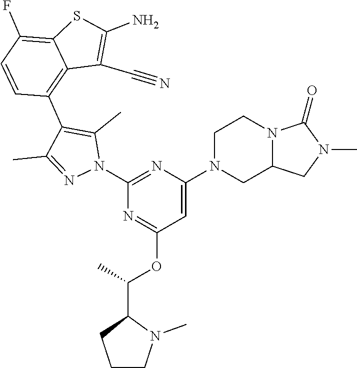 [SMILES: Cc1nn(-c2nc(O[C@@H](C)[C@@H]3CCCN3C)cc(N3CCN4C(=O)N(C)CC4C3)n2)c(C)c1-c1ccc(F)c2sc(N)c(C#N)c12] | 2-Amino-4-(3,5-dimethyl-1-(4-(2-methyl-3- oxohexahydroimidazo[1,5-a]pyrazin-7(1H)-yl)- 6-((S)-1-((S)-1-methylpyrrolidin-2- yl)ethoxy)pyrimidin-2-yl)-1H-pyrazol-4-yl)-7- fluorobenzo[b]thiophene-3-carbonitrile | 645.3 |
| 10 | 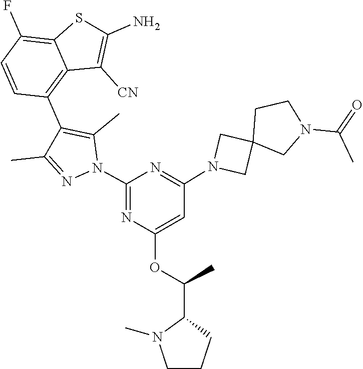 [SMILES: CC(=O)N1CCC2(C1)CN(c1cc(O[C@@H](C)[C@@H]3CCCN3C)nc(-n3nc(C)c(-c4ccc(F)c5sc(N)c(C#N)c45)c3C)n1)C2] | 4-(1-(4-(6-Acetyl-2,6-diazaspiro[3.4]octan-2-yl)- 6-((S)-1-((S)-1-methylpyrrolidin-2- yl)ethoxy)pyrimidin-2-yl)-3,5-dimethyl-1H- pyrazol-4-yl)-2-amino-7- fluorobenzo[b]thiophene-3-carbonitrile | 644.3 |
| 11 | 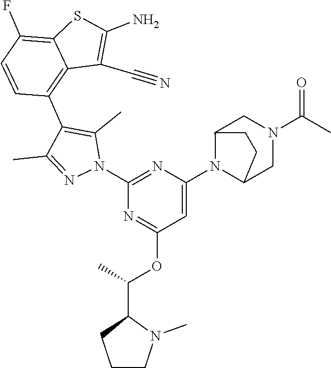 [SMILES: CC(=O)N1CC2CCC(C1)N2c1cc(O[C@@H](C)[C@@H]2CCCN2C)nc(-n2nc(C)c(-c3ccc(F)c4sc(N)c(C#N)c34)c2C)n1] | 4-(1-(4-(3-Acetyl-3,8-diazabicyclo[3.2.1]octan- 8-yl)-6-((S)-1-((S)-1-methylpyrrolidin-2- yl)ethoxy)pyrimidin-2-yl)-3,5-dimethyl-1H- pyrazol-4-yl)-2-amino-7- fluorobenzo[b]thiophene-3-carbonitrile | 644.3 |
| 12 | 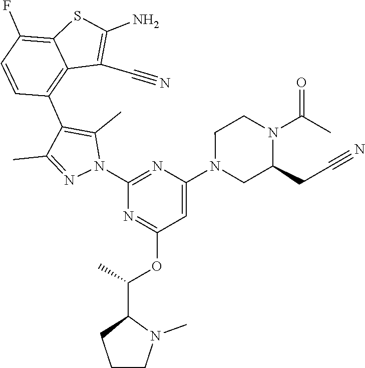 [SMILES: CC(=O)N1CCN(c2cc(O[C@@H](C)[C@@H]3CCCN3C)nc(-n3nc(C)c(-c4ccc(F)c5sc(N)c(C#N)c45)c3C)n2)C[C@@H]1CC#N] | 4-(1-(4-((S)-4-Acetyl-3-(cyanomethyl)piperazin- 1-yl)-6-((S)-1-((S)-1-methylpyrrolidin-2- yl)ethoxy)pyrimidin-2-yl)-3,5-dimethyl-1H- pyrazol-4-yl)-2-amino-7- fluorobenzo[b]thiophene-3-carbonitrile | 657.3 |
| 13 | 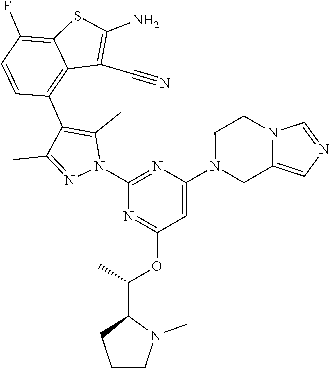 [SMILES: Cc1nn(-c2nc(O[C@@H](C)[C@@H]3CCCN3C)cc(N3CCn4cncc4C3)n2)c(C)c1-c1ccc(F)c2sc(N)c(C#N)c12] | 2-Amino-4-(1-(4-(5,6-dihydroimidazo[1,5- a]pyrazin-7(8H)-yl)-6-((S)-1-((S)-1- methylpyrrolidin-2-yl)ethoxy)pyrimidin-2-yl)-3,5- dimethyl-1H-pyrazol-4-yl)-7- fluorobenzo[b]thiophene-3-carbonitrile | 613.2 |
| 14 | 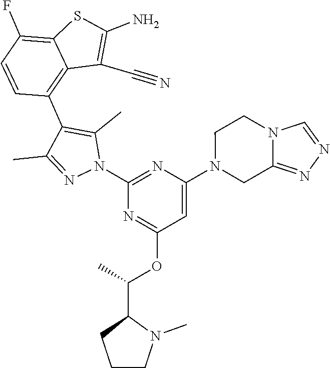 [SMILES: Cc1nn(-c2nc(O[C@@H](C)[C@@H]3CCCN3C)cc(N3CCn4cnnc4C3)n2)c(C)c1-c1ccc(F)c2sc(N)c(C#N)c12] | 2-Amino-4-(1-(4-(5,6-dihydro- [1,2,4]triazolo[4,3-a]pyrazin-7(8H)-yl)-6-((S)-1- ((S)-1-methylpyrrolidin-2-yl)ethoxy)pyrimidin-2- yl)-3,5-dimethyl-1H-pyrazol-4-yl)-7- fluorobenzo[b]thiophene-3-carbonitrile | 614.2 |
| 15 | 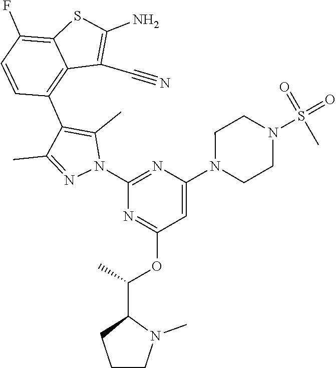 [SMILES: Cc1nn(-c2nc(O[C@@H](C)[C@@H]3CCCN3C)cc(N3CCN(S(C)(=O)=O)CC3)n2)c(C)c1-c1ccc(F)c2sc(N)c(C#N)c12] | 2-Amino-4-(3,5-dimethyl-1-(4-((S)-1-((S)-1- methylpyrrolidin-2-yl)ethoxy)-6-(4- (methylsulfonyl)piperazin-1-yl)pyrimidin-2-yl)- 1H-pyrazol-4-yl)-7-fluorobenzo[b]thiophene-3- carbonitrile | 654.2 |
| 16 | 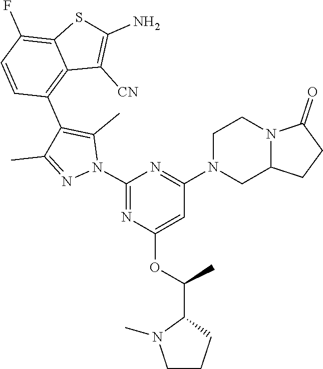 [SMILES: Cc1nn(-c2nc(O[C@@H](C)[C@@H]3CCCN3C)cc(N3CCN4C(=O)CCC4C3)n2)c(C)c1-c1ccc(F)c2sc(N)c(C#N)c12] | 2-Amino-4-(3,5-dimethyl-1-(4-((S)-1-((S)-1- methylpyrrolidin-2-yl)ethoxy)-6-(6- oxohexahydropyrrolo[1,2-a]pyrazin-2(1H)- yl)pyrimidin-2-yl)-1H-pyrazol-4-yl)-7- fluorobenzo[b]thiophene-3-carbonitrile | 630.3 |
| 17 | 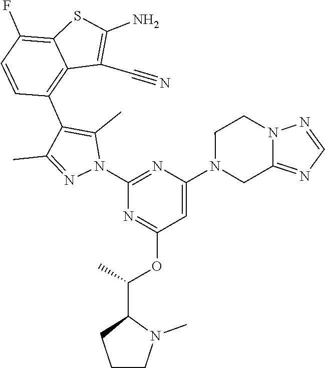 [SMILES: Cc1nn(-c2nc(O[C@@H](C)[C@@H]3CCCN3C)cc(N3CCn4ncnc4C3)n2)c(C)c1-c1ccc(F)c2sc(N)c(C#N)c12] | 2-Amino-4-(1-(4-(5,6-dihydro- [1,2,4]triazolo[1,5-a]pyrazin-7(8H)-yl)-6-((S)-1- ((S)-1-methylpyrrolidin-2-yl)ethoxy)pyrimidin-2- yl)-3,5-dimethyl-1H-pyrazol-4-yl)-7- fluorobenzo[b]thiophene-3-carbonitrile | 614.2 |

Diastereomers of Examples 2-17 are understood to be as listed for each Example below:

### Example 2

1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpyrrolidine-3-carboxamide; and
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpyrrolidine-3-carboxamide.

### Example 3

2-amino-(Rₐ)-4-(3,5-dimethyl-1-(4-(4-methyl-3-oxopiperazin-1-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile; and
2-amino-(Sₐ)-4-(3,5-dimethyl-1-(4-(4-methyl-3-oxopiperazin-1-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile.

### Example 4

2-amino-7-fluoro-(Rₐ)-4-(1-(4-(4-hydroxy-4-methylpiperidin-1-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile; and
2-amino-7-fluoro-(Sₐ)-4-(1-(4-(4-hydroxy-4-methylpiperidin-1-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile.

### Example 5

4-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpiperazine-1-carboxamide; and
4-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpiperazine-1-carboxamide.

### Example 6

(Rₐ)-4-(1-(4-(6-acetyl-2,6-diazaspiro[3.3]heptan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile; and
(Sₐ)-4-(1-(4-(6-acetyl-2,6-diazaspiro[3.3]heptan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile.

### Example 7

2-amino-(Rₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile.

### Example 8

methyl 4-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)piperazine-1-carboxylate;
methyl 4-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)piperazine-1-carboxylate.

### Example 9

2-amino-(Rₐ)-4-(3,5-dimethyl-1-(4-(2-methyl-3-oxohexahydroimidazo[1,5-a]pyrazin-7(1H)-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(2-methyl-3-oxohexahydroimidazo[1,5-a]pyrazin-7(1H)-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile.

### Example 10

(Rₐ)-4-(1-(4-(6-acetyl-2,6-diazaspiro[3.4]octan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(6-acetyl-2,6-diazaspiro[3.4]octan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile.

### Example 11

(Rₐ)-4-(1-(4-((1R,5S)-3-acetyl-3,8-diazabicyclo[3.2.1]octan-8-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1R,5S)-3-acetyl-3,8-diazabicyclo[3.2.1]octan-8-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile.

### Example 12

(Rₐ)-4-(1-(4-((S)-4-acetyl-3-(cyanomethyl)piperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((S)-4-acetyl-3-(cyanomethyl)piperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile.

### Example 13

2-amino-(Rₐ)-4-(1-(4-(5,6-dihydroimidazo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydroimidazo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile.

### Example 14

2-amino-(Rₐ)-4-(1-(4-(5,6-dihydro-[1,2,4]triazolo[4,3-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[4,3-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile.

### Example 15

2-amino-(Rₐ)-4-(3,5-dimethyl-1-(4-((S)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile.

### Example 16

2-amino-(Rₐ)-4-(3,5-dimethyl-1-(4-((S)-1-((S)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile.

### Example 17

2-amino-(Rₐ)-4-(1-(4-(5,6-dihydro-[1,2,4]triazolo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile.

### Example 18: 2-Amino-4-(3,5-dimethyl-1-(4-((S)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile

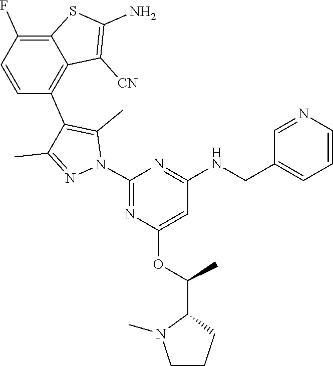 [SMILES: Cc1nn(-c2nc(NCc3cccnc3)cc(O[C@@H](C)[C@@H]3CCCN3C)n2)c(C)c1-c1ccc(F)c2sc(N)c(C#N)c12]

A mixture of Intermediate 2 (20 mg, 0.032 mmol), pyridine-3-ylmethanamine (10.36 mg, 0.096 mmol), BrettPhos Pd G3 (8.69 mg, 9.58 μmol), and sodium tert-butoxide (15.35 mg, 0.160 mmol) in 1,4-dioxane (0.8 mL) was bubbled with N₂ gas for 5 min. Then the mixture was stirred at 90° C. for 2 h. After cooling to r.t., the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₁H₃₃FN₉OS (M+H)⁺: m/z=598.2; found 598.3. * This peak is the more potent one.

Peak 2, diastereomer 2. LC-MS calc. for C₃₁H₃₃FN₉OS (M+H)⁺: m/z=598.2; found 598.3.

Diastereomers of Example 18 are understood to be:
2-amino-(Rₐ)-4-(3,5-dimethyl-1-(4-((S)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile.

### Examples 19-20

The following compounds listed in Table 5 were prepared in an analogous fashion to Example 18 using the appropriate coupling partner.

TABLE 5
Examples 19-20

| Ex | | | ESI |
|---|---|---|---|
| # | Structure | IUPAC name | [M + H]⁺ |
| 19 | 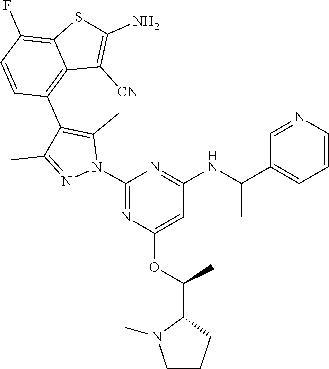 [SMILES: Cc1nn(-c2nc(NC(C)c3cccnc3)cc(O[C@@H](C)[C@@H]3CCCN3C)n2)c(C)c1-c1ccc(F)c2sc(N)c(C#N)c12] | 2-Amino-4-(3,5-dimethyl-1-(4-((S)-1- ((S)-1-methylpyrrolidin-2-yl)ethoxy)-6- ((1-(pyridin-3-yl)ethyl)amino)pyrimidin- 2-yl)-1H-pyrazol-4-yl)-7- fluorobenzo[b]thiophene-3-carbonitrile | 612.3 |
| 20 | 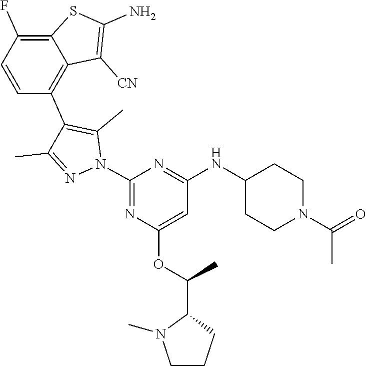 [SMILES: CC(=O)N1CCC(Nc2cc(O[C@@H](C)[C@@H]3CCCN3C)nc(-n3nc(C)c(-c4ccc(F)c5sc(N)c(C#N)c45)c3C)n2)CC1] | 4-(1-(4-((1-Acetylpiperidin-4-yl)amino)- 6-((S)-1-((S)-1-methylpyrrolidin-2- yl)ethoxy)pyrimidin-2-yl)-3,5-dimethyl- 1H-pyrazol-4-yl)-2-amino-7- fluorobenzo[b]thiophene-3-carbonitrile | 632.3 |

Diastereomers of Examples 19 and 20 are understood to be:

### Example 19

2-amino-(Rₐ)-4-(3,5-dimethyl-1-(4-((S)-1-((S)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile.

### Example 20

(Rₐ)-4-(1-(4-((1-acetylpiperidin-4-yl)amino)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)amino)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile.

### Example 21: 2-Amino-4-(3,5-dimethyl-1-(4-(1-methyl-1H-pyrazol-4-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile

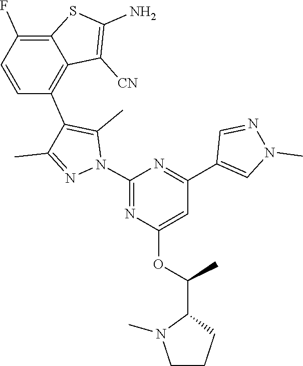 [SMILES: Cc1nn(-c2nc(O[C@@H](C)[C@@H]3CCCN3C)cc(-c3cnn(C)c3)n2)c(C)c1-c1ccc(F)c2sc(N)c(C#N)c12]

A mixture of Intermediate 2 (16 mg, 0.026 mmol), 1-methyl-4-(4,4,5,5-tetramethyl-1,3,2-dioxaborolan-2-yl)-1H-pyrazole (15.95 mg, 0.077 mmol), XPhos Pd G2 (4.02 mg, 5.11 μmol) and tripotassium phosphate (16.27 mg, 0.077 mmol) in 1,4-dioxane (0.8 mL) and water (0.160 mL) was stirred at 100° C. under an atmosphere of N₂, and the reaction was monitored with LCMS. Upon completion, the mixture was cooled to r.t., and the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₉H₃₁FN₉OS (M+H)⁺: m/z=572.2; found 572.2. * This peak is the more potent one.

Peak 2, diastereomer 2. LC-MS calc. for C₂₉H₃₁FN₉OS (M+H)⁺: m/z=572.2; found 572.2.

Diastereomers of Example 21 are understood to be:
2-amino-(Rₐ)-4-(3,5-dimethyl-1-(4-(1-methyl-1H-pyrazol-4-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-4-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile.

### Examples 22-30

The following compounds listed in Table 6 were prepared in an analogous fashion to Example 21 using the appropriate coupling partner.

TABLE 6
Examples 22-30

| Ex | | | ESI |
|---|---|---|---|
| # | Structure | IUPAC name | [M + H]⁺ |
| 22 | 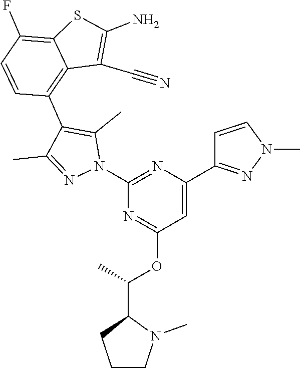 [SMILES: Cc1nn(-c2nc(O[C@@H](C)[C@@H]3CCCN3C)cc(-c3ccn(C)n3)n2)c(C)c1-c1ccc(F)c2sc(N)c(C#N)c12] | 2-Amino-4-(3,5-dimethyl-1-(4-(1-methyl- 1H-pyrazol-3-yl)-6-((S)-1-((S)-1- methylpyrrolidin-2-yl)ethoxy)pyrimidin-2- yl)-1H-pyrazol-4-yl)-7- fluorobenzo[b]thiophene-3-carbonitrile | 572.2 |
| 23 | 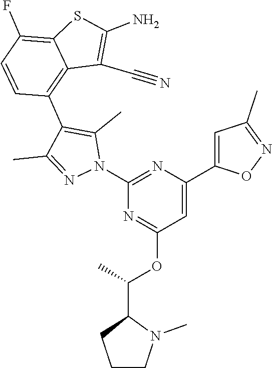 [SMILES: Cc1cc(-c2cc(O[C@@H](C)[C@@H]3CCCN3C)nc(-n3nc(C)c(-c4ccc(F)c5sc(N)c(C#N)c45)c3C)n2)on1] | 2-Amino-4-(3,5-dimethyl-1-(4-(3- methylisoxazol-5-yl)-6-((S)-1-((S)-1- methylpyrrolidin-2-yl)ethoxy)pyrimidin-2- yl)-1H-pyrazol-4-yl)-7- fluorobenzo[b]thiophene-3-carbonitrile | 573.2 |
| 24 | 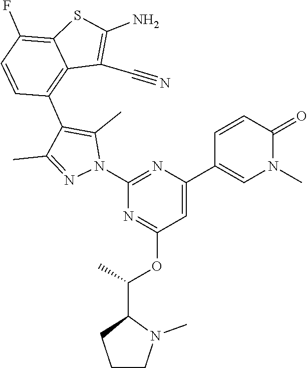 [SMILES: Cc1nn(-c2nc(O[C@@H](C)[C@@H]3CCCN3C)cc(-c3ccc(=O)n(C)c3)n2)c(C)c1-c1ccc(F)c2sc(N)c(C#N)c12] | 2-Amino-4-(3,5-dimethyl-1-(4-(1-methyl- 6-oxo-1,6-dihydropyridin-3-yl)-6-((S)-1- ((S)-1-methylpyrrolidin-2- yl)ethoxy)pyrimidin-2-yl)-1H-pyrazol-4- yl)-7-fluorobenzo[b]thiophene-3- carbonitrile | 599.2 |
| 25 | 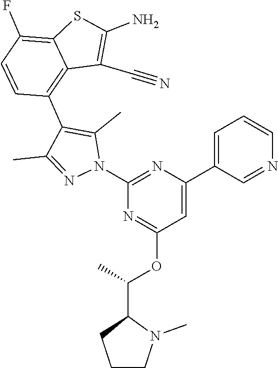 [SMILES: Cc1nn(-c2nc(O[C@@H](C)[C@@H]3CCCN3C)cc(-c3cccnc3)n2)c(C)c1-c1ccc(F)c2sc(N)c(C#N)c12] | 2-Amino-4-(3,5-dimethyl-1-(4-((S)-1-((S)- 1-methylpyrrolidin-2-yl)ethoxy)-6- (pyridin-3-yl)pyrimidin-2-yl)-1H-pyrazol- 4-yl)-7-fluorobenzo[b]thiophene-3- carbonitrile | 569.2 |
| 26 | 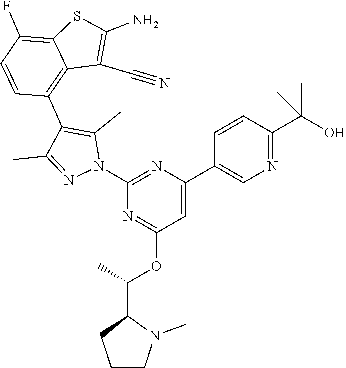 [SMILES: Cc1nn(-c2nc(O[C@@H](C)[C@@H]3CCCN3C)cc(-c3ccc(C(C)(C)O)nc3)n2)c(C)c1-c1ccc(F)c2sc(N)c(C#N)c12] | 2-Amino-7-fluoro-4-(1-(4-(6-(2- hydroxypropan-2-yl)pyridin-3-yl)-6-((S)- 1-((S)-1-methylpyrrolidin-2- yl)ethoxy)pyrimidin-2-yl)-3,5-dimethyl- 1H-pyrazol-4-yl)benzo[b]thiophene-3- carbonitrile | 627.3 |
| 27 | 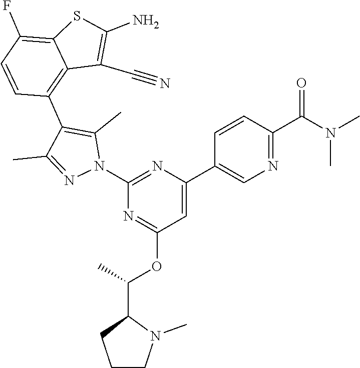 [SMILES: Cc1nn(-c2nc(O[C@@H](C)[C@@H]3CCCN3C)cc(-c3ccc(C(=O)N(C)C)nc3)n2)c(C)c1-c1ccc(F)c2sc(N)c(C#N)c12] | 5-(2-(4-(2-Amino-3-cyano-7- fluorobenzo[b]thiophen-4-yl)-3,5- dimethyl-1H-pyrazol-1-yl)-6-((S)-1-((S)- 1-methylpyrrolidin-2-yl)ethoxy)pyrimidin- 4-yl)-N,N-dimethylpicolinamide | 640.3 |
| 28 | 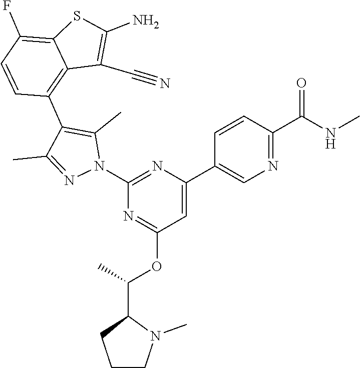 [SMILES: CNC(=O)c1ccc(-c2cc(O[C@@H](C)[C@@H]3CCCN3C)nc(-n3nc(C)c(-c4ccc(F)c5sc(N)c(C#N)c45)c3C)n2)cn1] | 5-(2-(4-(2-Amino-3-cyano-7- fluorobenzo[b]thiophen-4-yl)-3,5- dimethyl-1H-pyrazol-1-yl)-6-((S)-1-((S)- 1-methylpyrrolidin-2-yl)ethoxy)pyrimidin- 4-yl)-N-methylpicolinamide | 626.2 |
| 29 | 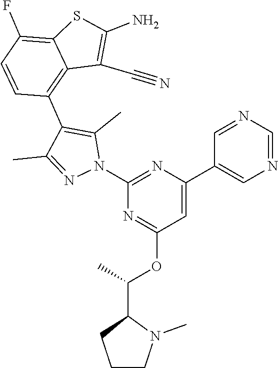 [SMILES: Cc1nn(-c2nc(O[C@@H](C)[C@@H]3CCCN3C)cc(-c3cncnc3)n2)c(C)c1-c1ccc(F)c2sc(N)c(C#N)c12] | 2-Amino-4-(3,5-dimethyl-1-(6-((S)-1-((S)- 1-methylpyrrolidin-2-yl)ethoxy)-[4,5′- bipyrimidin]-2-yl)-1H-pyrazol-4-yl)-7- fluorobenzo[b]thiophene-3-carbonitrile | 570.2 |
| 30 | 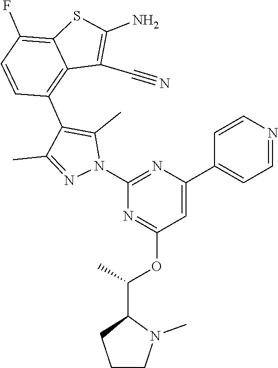 [SMILES: Cc1nn(-c2nc(O[C@@H](C)[C@@H]3CCCN3C)cc(-c3ccncc3)n2)c(C)c1-c1ccc(F)c2sc(N)c(C#N)c12] | 2-amino-4-(3,5-dimethyl-1-(4-((S)-1-((S)- 1-methylpyrrolidin-2-yl)ethoxy)-6- (pyridin-4-yl)pyrimidin-2-yl)-1H-pyrazol- 4-yl)-7-fluorobenzo[b]thiophene-3- carbonitrile | 569.2 |

Diastereomers of the compounds of Table 6 are understood to be as listed below for each Example:

### Example 22

2-amino-(Rₐ)-4-(3,5-dimethyl-1-(4-(1-methyl-1H-pyrazol-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile.

### Example 23

2-amino-(Rₐ)-4-(3,5-dimethyl-1-(4-(3-methylisoxazol-5-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(3-methylisoxazol-5-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile.

### Example 24

2-amino-(Rₐ)-4-(3,5-dimethyl-1-(4-(1-methyl-6-oxo-1,6-dihydropyridin-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-6-oxo-1,6-dihydropyridin-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile.

### Example 25

2-amino-(Rₐ)-4-(3,5-dimethyl-1-(4-((S)-1-((S)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile.

### Example 26

2-amino-7-fluoro-(Rₐ)-4-(1-(4-(6-(2-hydroxypropan-2-yl)pyridin-3-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(6-(2-hydroxypropan-2-yl)pyridin-3-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile.

### Example 27

5-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpicolinamide;
5-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpicolinamide.

### Example 28

5-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpicolinamide;
5-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpicolinamide.

### Example 29

2-amino-(Rₐ)-4-(3,5-dimethyl-1-(6-((S)-1-((S)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Sₐ)-4-(3,5-dimethyl-1-(6-((S)-1-((S)-1-methylpyrrolidin-2-yl)ethoxy)-[4,5′-bipyrimidin]-2-yl)-1H-pyrazol-4-yl)-7-fluorobenzo[b]thiophene-3-carbonitrile.

### Example 30

2-amino-(Rₐ)-4-(3,5-dimethyl-1-(4-((S)-1-((S)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile.

### Example 31. 1-(2-(4-(2-Amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-1H-pyrazole-3-carboxamide

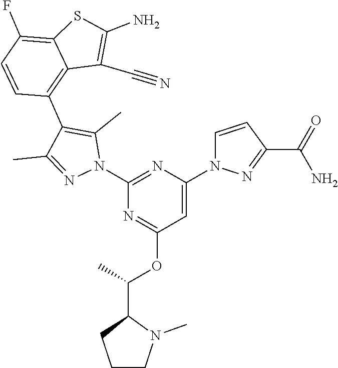 [SMILES: Cc1nn(-c2nc(O[C@@H](C)[C@@H]3CCCN3C)cc(-n3ccc(C(N)=O)n3)n2)c(C)c1-c1ccc(F)c2sc(N)c(C#N)c12]

A mixture of Intermediate 2 (10 mg, 0.016 mmol), 1H-pyrozole-3-carboxamide (5.32 mg, 0.048 mmol) and Cs₂CO₃ (15.61 mg, 0.048 mmol) in MeCN (0.5 mL) was stirred at 90° C. for 3 h. The mixture was cooled to r.t, and the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₉H₃₀FN₁₀O₃S (M+H)⁺: m/z=601.2; found 601.2. * This peak is the more potent one.

Peak 2, diastereomer 2. LC-MS calc. for C₂₉H₃₀FN₁₀O₃S (M+H)⁺: m/z=601.2; found 601.2.

Diastereomers of Example 31 are understood to be:
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-1H-pyrazole-3-carboxamide.

### Examples 32-34

The following compounds listed in Table 7 were prepared in an analogous fashion to Example 31 using the appropriate coupling partner.

TABLE 7
Examples 32-34

| Ex | | | ESI |
|---|---|---|---|
| # | Structure | IUPAC name | [M + H]⁺ |
| 32 | 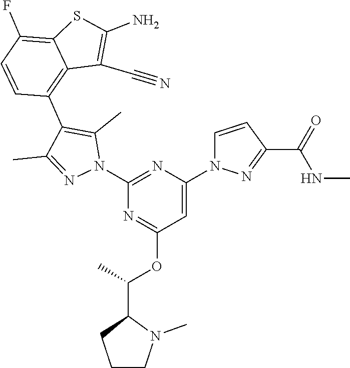 [SMILES: CNC(=O)c1ccn(-c2cc(O[C@@H](C)[C@@H]3CCCN3C)nc(-n3nc(C)c(-c4ccc(F)c5sc(N)c(C#N)c45)c3C)n2)n1] | 1-(2-(4-(2-Amino-3-cyano-7- fluorobenzo[b]thiophen-4-yl)- 3,5-dimethyl-1H-pyrazol-1-yl)-6- ((S)-1-((S)-1-methylpyrrolidin-2- yl)ethoxy)pyrimidin-4-yl)-N- methyl-1H-pyrazole-3- carboxamide | 615.2 |
| 33 | 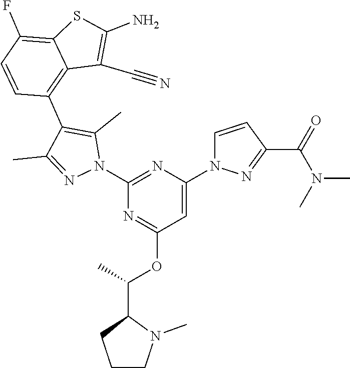 [SMILES: Cc1nn(-c2nc(O[C@@H](C)[C@@H]3CCCN3C)cc(-n3ccc(C(=O)N(C)C)n3)n2)c(C)c1-c1ccc(F)c2sc(N)c(C#N)c12] | 1-(2-(4-(2-amino-3-cyano-7- fluorobenzo[b]thiophen-4-yl)- 3,5-dimethyl-1H-pyrazol-1-yl)-6- ((S)-1-((S)-1-methylpyrrolidin-2- yl)ethoxy)pyrimidin-4-yl)-N,N- dimethyl-1H-pyrazole-3- carboxamide | 629.2 |
| 34 | 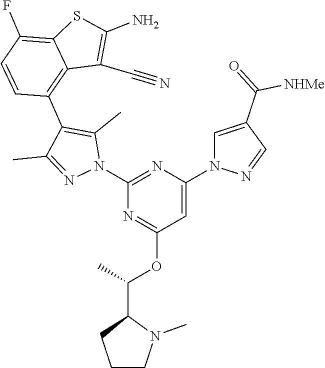 [SMILES: CNC(=O)c1cnn(-c2cc(O[C@@H](C)[C@@H]3CCCN3C)nc(-n3nc(C)c(-c4ccc(F)c5sc(N)c(C#N)c45)c3C)n2)c1] | 1-(2-(4-(2-Amino-3-cyano-7- fluorobenzo[b]thiophen-4-yl)- 3,5-dimethyl-1H-pyrazol-1-yl)-6- ((S)-1-((S)-1-methylpyrrolidin-2- yl)ethoxy)pyrimidin-4-yl)-N- methyl-1H-pyrazole-4- carboxamide | 615.2 |

Diastereomers of Examples 32-34 are understood to be:

### Example 32

1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-3-carboxamide;
1-(2-((Sₐ)-4(2-amino-3-cyano-7-fluorobenzo[b]thiophene-4-yl)-3,5-dimethyl- H-pyrazol-1-yl)-6-((S)-1-((S)-1-methylpyrrolidin-2-yl)ethoxy)pyrimidin-4-yl)-N-methyl-1H-pyrazole-3-carboxamide.

### Example 33

1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethyl-1H-pyrazole-3-carboxamide;
1-(2-((Sₐ)-4(2-amino-3-cyano-7-fluorobenzo[b]thiophene-4-yl)-3,5-dimethyl-H-pyrazol-1-yl)-6-((S)-1-((S)-1-methylpyrrolidin-2-yl)ethoxy)pyrimidin-4-yl)-N,N-dimethyl-1H-pyrazole-3-carboxamide.

### Example 34

1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-4-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-4-carboxamide.

### Example 35. 2-Amino-4-(3,5-dimethyl-1-(4-((S)-1-((S)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile

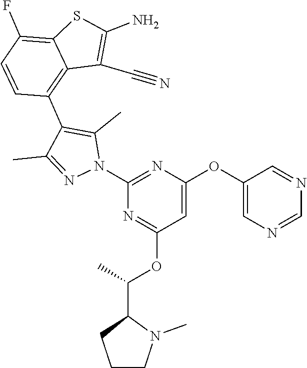 [SMILES: Cc1nn(-c2nc(Oc3cncnc3)cc(O[C@@H](C)[C@@H]3CCCN3C)n2)c(C)c1-c1ccc(F)c2sc(N)c(C#N)c12]

A mixture of Intermediate 2 (10 mg, 0.016 mmol), pyrimidin-5-ol (4.60 mg, 0.048 mmol) and Cs₂CO₃ (15.61 mg, 0.048 mmol) in Acetonitrile (0.5 mL) was stirred at 90° C. for 3 h. The mixture was cooled to r.t., and the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₉H₂₉FN₉O₂S (M+H)⁺: m/z=586.2; found 586.2. * This peak is the more potent one.

Peak 2, diastereomer 2. LC-MS calc. for C₂₉H₂₉FN₉O₂S (M+H)⁺: m/z=586.2; found 586.2.

Diastereomers of Example 35 are understood to be:
2-amino-(Rₐ)-4-(3,5-dimethyl-1-(4-((S)-1-((S)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile.

### Example 36. 4-(1-(4-((1-Acetylpiperidin-4-yl)oxy)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile

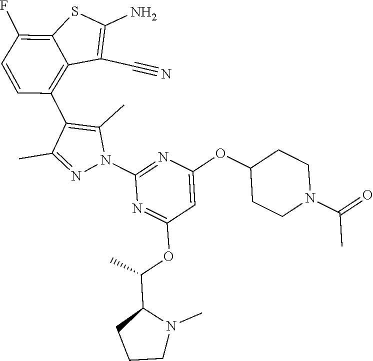 [SMILES: CC(=O)N1CCC(Oc2cc(O[C@@H](C)[C@@H]3CCCN3C)nc(-n3nc(C)c(-c4ccc(F)c5sc(N)c(C#N)c45)c3C)n2)CC1]

Sodium hydride (60 wt % in mineral oil, 1.92 mg, 0.048 mmol) was added to a solution of 1-(4-hydroxypiperdin-1-yl)ethan-1-one (6.86 mg, 0.048 mmol) in THF (0.5 mL) at r.t. After stirring at r.t. for 30 min., Intermediate 2 (10 mg, 0.016 mmol) was added to the reaction, and stirring was continued at r.t. for 1 h. The volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₂H₃₈FN₈O₃S (M+H)⁺: m/z=633.3; found 633.3.* This peak is the more potent one.

Peak 2, diastereomer 2. LC-MS calc. for C₃₂H₃₈FN₈O₃S (M+H)⁺: m/z=633.3; found 633.3.

Diastereomers of Example 36 are understood to be:
(Rₐ)-4-(1-(4-((1-acetylpiperidin-4-yl)oxy)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)oxy)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile.

### Example 37: 2-Amino-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

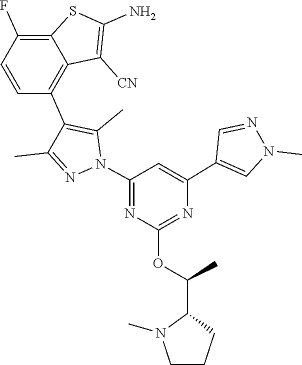 [SMILES: Cc1nn(-c2cc(-c3cnn(C)c3)nc(O[C@@H](C)[C@@H]3CCCN3C)n2)c(C)c1-c1ccc(F)c2sc(N)c(C#N)c12]

A mixture of Intermediate 3 (16 mg, 0.026 mmol), 1-methyl-4-(4,4,5,5-tetramethyl-1,3,2-dioxaborolan-2-yl)-1H-pyrazole (15.95 mg, 0.077 mmol), XPhos Pd G2 (4.02 mg, 5.11 μmol) and tripotassium phosphate (16.27 mg, 0.077 mmol) in 1,4-dioxane (0.8 mL) and water (0.160 mL) was stirred at 100° C., and the reaction was monitored with LCMS. Upon completion, the mixture was cooled to r.t., and the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₉H₃₁FN₉OS (M+H)⁺: m/z=572.2; found 572.3.

Peak 2, diastereomer 2. LC-MS calc. for C₂₉H₃₁FN₉OS (M+H)⁺: m/z=572.2; found 572.3. * This peak is the more potent one. ¹H NMR (500 MHz, DMSO) δ 9.92-9.66 (m, 1H, TFA proton), 8.62 (s, 1H), 8.20 (s, 1H), 8.01 (s, 2H), 7.81 (s, 1H), 7.18-7.06 (m, 2H), 5.23 (dq, J=9.3, 6.1 Hz, 1H), 3.93 (s, 3H), 3.85-3.74 (m, 1H), 3.60-3.45 (m, 1H), 3.22-3.10 (m, 1H), 3.03 (d, J=4.8 Hz, 3H), 2.51 (s, 3H), 2.34-2.23 (m, 1H), 2.14-2.02 (m, 4H), 1.98-1.88 (m, 1H), 1.86-1.76 (m, 1H), 1.44 (d, J=6.1 Hz, 3H).

Diastereomers of Example 37 are understood to be:
2-amino-(Rₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Examples 38-46

The following compounds listed in Table 8 were prepared in an analogous fashion to Example 37 using the appropriate coupling partner.

TABLE 8
Examples 38-46

| Ex | | | ESI |
|---|---|---|---|
| # | Structure | IUPAC name | [M + H]⁺ |
| 38 | 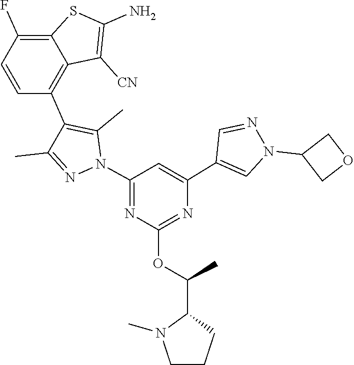 [SMILES: Cc1nn(-c2cc(-c3cnn(C4COC4)c3)nc(O[C@@H](C)[C@@H]3CCCN3C)n2)c(C)c1-c1ccc(F)c2sc(N)c(C#N)c12] | 2-Amino-4-(3,5-dimethyl-1-(2-((S)-1- ((S)-1-methylpyrrolidin-2-yl)ethoxy)-6- (1-(oxetan-3-yl)-1H-pyrazol-4- yl)pyrimidin-4-yl)-1H-pyrazol-4-yl)-7- fluorobenzo[b]thiophene-3-carbonitrile | 614.2 |
| 39 | 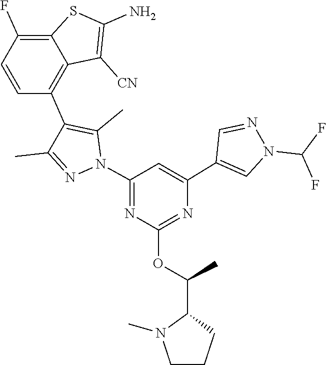 [SMILES: Cc1nn(-c2cc(-c3cnn(C(F)F)c3)nc(O[C@@H](C)[C@@H]3CCCN3C)n2)c(C)c1-c1ccc(F)c2sc(N)c(C#N)c12] | 2-Amino-4-(1-(6-(1-(difluoromethyl)- 1H-pyrazol-4-yl)-2-((S)-1-((S)-1- methylpyrrolidin-2-yl)ethoxy)pyrimidin- 4-yl)-3,5-dimethyl-1H-pyrazol-4-yl)-7- fluorobenzo[b]thiophene-3-carbonitrile | 608.2 |
| 40 | 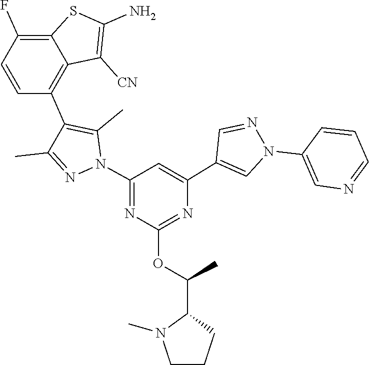 [SMILES: Cc1nn(-c2cc(-c3cnn(-c4cccnc4)c3)nc(O[C@@H](C)[C@@H]3CCCN3C)n2)c(C)c1-c1ccc(F)c2sc(N)c(C#N)c12] | 2-Amino-4-(3,5-dimethyl-1-(2-((S)-1- ((S)-1-methylpyrrolidin-2-yl)ethoxy)-6- (1-(pyridin-3-yl)-1H-pyrazol-4- yl)pyrimidin-4-yl)-1H-pyrazol-4-yl)-7- fluorobenzo[b]thiophene-3-carbonitrile | 635.2 |
| 41 | 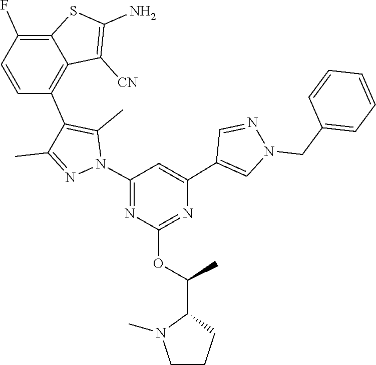 [SMILES: Cc1nn(-c2cc(-c3cnn(Cc4ccccc4)c3)nc(O[C@@H](C)[C@@H]3CCCN3C)n2)c(C)c1-c1ccc(F)c2sc(N)c(C#N)c12] | 2-Amino-4-(1-(6-(1-benzyl-1H-pyrazol- 4-yl)-2-((S)-1-((S)-1-methylpyrrolidin-2- yl)ethoxy)pyrimidin-4-yl)-3,5-dimethyl- 1H-pyrazol-4-yl)-7- fluorobenzo[b]thiophene-3-carbonitrile | 648.3 |
| 42 | 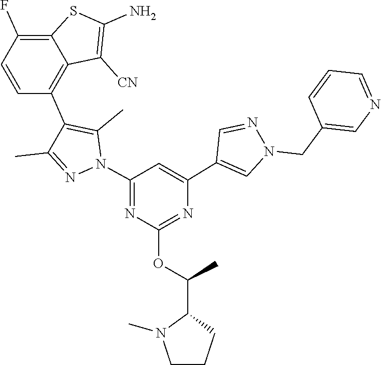 [SMILES: Cc1nn(-c2cc(-c3cnn(Cc4cccnc4)c3)nc(O[C@@H](C)[C@@H]3CCCN3C)n2)c(C)c1-c1ccc(F)c2sc(N)c(C#N)c12] | 2-Amino-4-(3,5-dimethyl-1-(2-((S)-1- ((S)-1-methylpyrrolidin-2-yl)ethoxy)-6- (1-(pyridin-3-ylmethyl)-1H-pyrazol-4- yl)pyrimidin-4-yl)-1H-pyrazol-4-yl)-7- fluorobenzo[b]thiophene-3-carbonitrile | 649.3 |
| 43 | 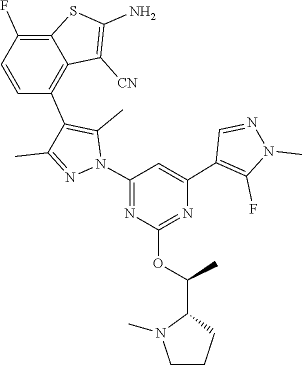 [SMILES: Cc1nn(-c2cc(-c3cnn(C)c3F)nc(O[C@@H](C)[C@@H]3CCCN3C)n2)c(C)c1-c1ccc(F)c2sc(N)c(C#N)c12] | 2-Amino-7-fluoro-4-(1-(6-(5-fluoro-1- methyl-1H-pyrazol-4-yl)-2-((S)-1-((S)- 1-methylpyrrolidin-2- yl)ethoxy)pyrimidin-4-yl)-3,5-dimethyl- 1H-pyrazol-4-yl)benzo[b]thiophene-3- carbonitrile | 590.2 |
| 44 | 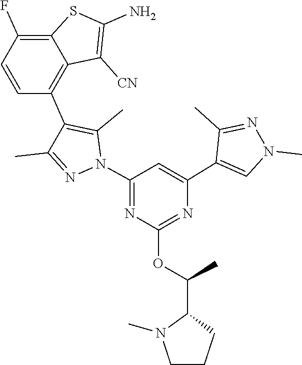 [SMILES: Cc1nn(C)cc1-c1cc(-n2nc(C)c(-c3ccc(F)c4sc(N)c(C#N)c34)c2C)nc(O[C@@H](C)[C@@H]2CCCN2C)n1] | 2-Amino-4-(1-(6-(1,3-dimethyl-1H- pyrazol-4-yl)-2-((S)-1-((S)-1- methylpyrrolidin-2-yl)ethoxy)pyrimidin- 4-yl)-3,5-dimethyl-1H-pyrazol-4-yl)-7- fluorobenzo[b]thiophene-3-carbonitrile | 586.2 |
| 45 | 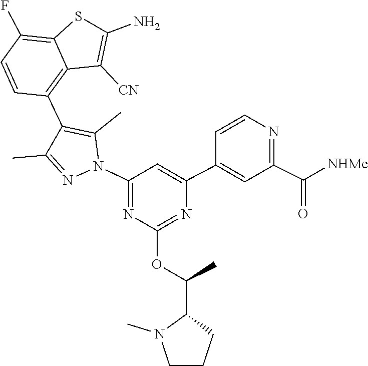 [SMILES: CNC(=O)c1cc(-c2cc(-n3nc(C)c(-c4ccc(F)c5sc(N)c(C#N)c45)c3C)nc(O[C@@H](C)[C@@H]3CCCN3C)n2)ccn1] | 4-(6-(4-(2-Amino-3-cyano-7- fluorobenzo[b]thiophen-4-yl)-3,5- dimethyl-1H-pyrazol-1-yl)-2-((S)-1-((S)- 1-methylpyrrolidin-2- yl)ethoxy)pyrimidin-4-yl)-N- methylpicolinamide | 626.2 |
| 46 | 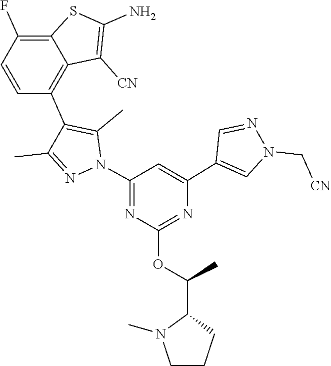 [SMILES: Cc1nn(-c2cc(-c3cnn(CC#N)c3)nc(O[C@@H](C)[C@@H]3CCCN3C)n2)c(C)c1-c1ccc(F)c2sc(N)c(C#N)c12] | 2-Amino-4-(1-(6-(1-(cyanomethyl)-1H- pyrazol-4-yl)-2-((S)-1-((S)-1- methylpyrrolidin-2-yl)ethoxy)pyrimidin- 4-yl)-3,5-dimethyl-1H-pyrazol-4-yl)-7- fluorobenzo[b]thiophene-3-carbonitrile | 597.2 |

Diastereomers of Examples 38-46 are understood to be as listed below for each Example:

### Example 38

2-amino-(Rₐ)-4-(3,5-dimethyl-1-(2-((S)-1-((S)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile.

### Example 39

2-amino-(Rₐ)-4-(1-(6-(1-(difluoromethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(difluoromethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile.

### Example 40

2-amino-(Rₐ)-4-(3,5-dimethyl-1-(2-((S)-1-((S)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile.

### Example 41

2-amino-(Rₐ)-4-(1-(6-(1-benzyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-benzyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile.

### Example 42

2-amino-(Rₐ)-4-(3,5-dimethyl-1-(2-((S)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile.

### Example 43

2-amino-7-fluoro-(Rₐ)-4-(1-(6-(5-fluoro-1-methyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-(5-fluoro-1-methyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile.

### Example 44

2-amino-(Rₐ)-4-(1-(6-(1,3-dimethyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1,3-dimethyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile.

### Example 45

4-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N-methylpicolinamide;
4-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N-methylpicolinamide.

### Example 46

2-amino-(Rₐ)-4-(1-(6-(1-(cyanomethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(cyanomethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile.

### Example 47: 2-Amino-4-(1-(6-(4-(cyclopropanecarbonyl)piperazin-1-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile

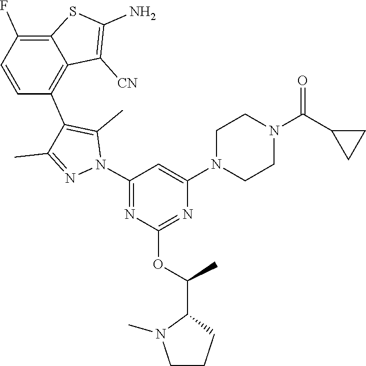 [SMILES: Cc1nn(-c2cc(N3CCN(C(=O)C4CC4)CC3)nc(O[C@@H](C)[C@@H]3CCCN3C)n2)c(C)c1-c1ccc(F)c2sc(N)c(C#N)c12]

A mixture of Intermediate 3 (10 mg, 0.016 mmol), cyclopropyl(piperazin-1-yl)methanone (7.39 mg, 0.048 mmol), RuPhos Pd G4 (2.72 mg, 3.19 μmol), RuPhos (2.98 mg, 6.39 μmol) and Cs₂CO₃ (15.61 mg, 0.048 mmol) in Dioxane (0.5 mL) was bubbled with N₂ gas for 5 min. Then the mixture was stirred at 90° C. for 2 h. After cooling to r.t., the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₃H₃₉FN₉O₂S (M+H)⁺: m/z=644.3; found 644.3.

Peak 2, diastereomer 2. LC-MS calc. for C₃₃H₃₉FN₉O₂S (M+H)⁺: m/z=644.3; found 644.3. * This peak is the more potent one.

Diastereomers of Example 47 are understood to be:
2-amino-(Rₐ)-4-(1-(6-(4-(cyclopropanecarbonyl)piperazin-1-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(4-(cyclopropanecarbonyl)piperazin-1-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile.

### Examples 48-49

The following compounds listed in Table 9 were prepared in an analogous fashion to Example 47 using the appropriate coupling partner.

TABLE 9
Examples 48-49

| Ex | | | ESI |
|---|---|---|---|
| # | Structure | IUPAC name | [M + H]⁺ |
| 48 | 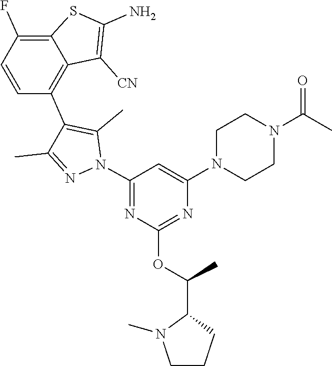 [SMILES: CC(=O)N1CCN(c2cc(-n3nc(C)c(-c4ccc(F)c5sc(N)c(C#N)c45)c3C)nc(O[C@@H](C)[C@@H]3CCCN3C)n2)CC1] | 4-(1-(6-(4-Acetylpiperazin-1-yl)-2-((S)-1-((S)- 1-methylpyrrolidin-2-yl)ethoxy)pyrimidin-4- yl)-3,5-dimethyl-1H-pyrazol-4-yl)-2-amino-7- fluorobenzo[b]thiophene-3-carbonitrile | 618.3 |
| 49 | 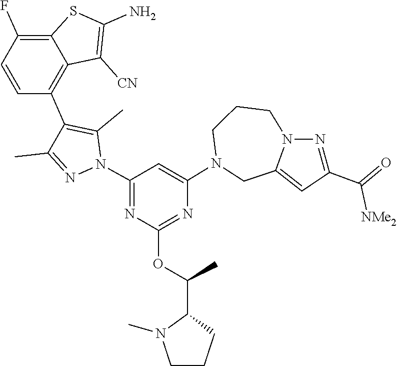 [SMILES: Cc1nn(-c2cc(N3CCCn4nc(C(=O)N(C)C)cc4C3)nc(O[C@@H](C)[C@@H]3CCCN3C)n2)c(C)c1-c1ccc(F)c2sc(N)c(C#N)c12] | 5-(6-(4-(2-Amino-3-cyano-7- fluorobenzo[b]thiophen-4-yl)-3,5-dimethyl- 1H-pyrazol-1-yl)-2-((S)-1-((S)-1- methylpyrrolidin-2-yl)ethoxy)pyrimidin-4-yl)- N,N-dimethyl-5,6,7,8-tetrahydro-4H- pyrazolo[1,5-a][1,4]diazepine-2- carboxamide | 698.3 |

Diastereomers of Examples 48 and 49 are understood to be:

### Example 48

(Rₐ)-4-(1-(6-(4-acetylpiperazin-1-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile.

### Example 49

5-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Sₐ)-4-(2-amino-3-cyano-7-fluorobenzo[b]thiophene-4yl)-3,5-dimethyl-H-pyrazol-1-yl)-2-((S)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide.

### Example 50. 1-(6-(4-(2-Amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-1H-pyrazole-3-carboxamide

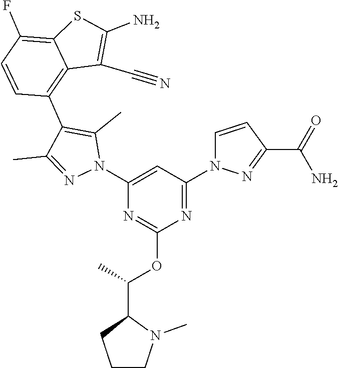 [SMILES: Cc1nn(-c2cc(-n3ccc(C(N)=O)n3)nc(O[C@@H](C)[C@@H]3CCCN3C)n2)c(C)c1-c1ccc(F)c2sc(N)c(C#N)c12]

A mixture of Intermediate 3 (10 mg, 0.016 mmol), 1H-pyrozole-3-carboxamide (5.32 mg, 0.048 mmol) and Cs₂CO₃ (15.61 mg, 0.048 mmol) in MeCN (0.5 mL) was stirred at 90° C. for 3 h. The mixture was cooled to r.t., and the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₉H₃₀FN₁₀O₃S (M+H)⁺: m/z=601.2; found 601.2.

Peak 2, diastereomer 2. LC-MS calc. for C₂₉H₃₀FN₁₀O₃S (M+H)⁺: m/z=601.2; found 601.2. * This peak is the more potent one.

Diastereomers of Example 50 are understood to be:
1-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-1H-pyrazole-3-carboxamide;
1-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-1H-pyrazole-3-carboxamide.

### Example 51. 4-(1-(6-(4-Acetylpiperazin-1-yl)-5-fluoro-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile

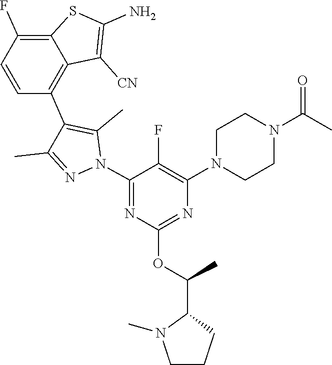 [SMILES: CC(=O)N1CCN(c2nc(O[C@@H](C)[C@@H]3CCCN3C)nc(-n3nc(C)c(-c4ccc(F)c5sc(N)c(C#N)c45)c3C)c2F)CC1]

### Step 1. tert-Butyl (4-(1-(6-(4-acetylpiperazin-1-yl)-5-fluoro-2-(methylthio)pyrimidin-4-yl)-3,5-dimethyl-1H-pyrazol-4-yl)-3-cyano-7-fluorobenzo[b]thiophene-2-yl)carbamate

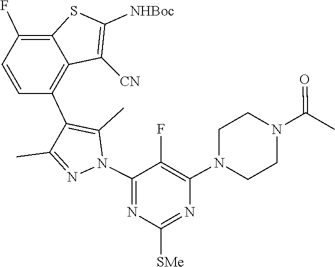 [SMILES: CSc1nc(N2CCN(C(C)=O)CC2)c(F)c(-n2nc(C)c(-c3ccc(F)c4sc(NC(=O)OC(C)(C)C)c(C#N)c34)c2C)n1]

Cs₂CO₃ (37.9 mg, 0.116 mmol) was added to a solution of Intermediate 1 (15 mg, 0.039 mmol) and 4,6-dichloro-5-fluoro-2-(methylthio)pyrimidine (12.40 mg, 0.058 mmol) in MeCN (0.8 mL), then the mixture was stirred at 80° C. for 1 h. After cooling to rt, 1-(piperazin-1-yl)ethan-1-one (14.93 mg, 0.116 mmol) was added to the reaction, and the resulting mixture was again stirred at 80° C. for another 1 h. The insoluble salts were filtered off, and the filtrate was concentrated in vacuo. The residue was purified by FCC (eluting with 0˜100% EtOAc in hexanes) to afford the title compound (20 mg, 79% yield). LC-MS calc. for C₃₀H₃₃F₂N₈O₃S₂ (M+H)⁺: m/z=655.2; found 655.2.

### Step 2. tert-Butyl (4-(1-(6-(4-acetylpiperazin-1-yl)-5-fluoro-2-(methylsulfinyl)pyrimidin-4-yl)-3,5-dimethyl-1H-pyrazol-4-yl)-3-cyano-7-fluorobenzo[b]thiophene-2-yl)carbamate and tert-butyl (4-(1-(6-(4-acetylpiperazin-1-yl)-5-fluoro-2-(methylsulfonyl)pyrimidin-4-yl)-3,5-dimethyl-1H-pyrazol-4-yl)-3-cyano-7-fluorobenzo[b]thiophene-2-yl)carbamate

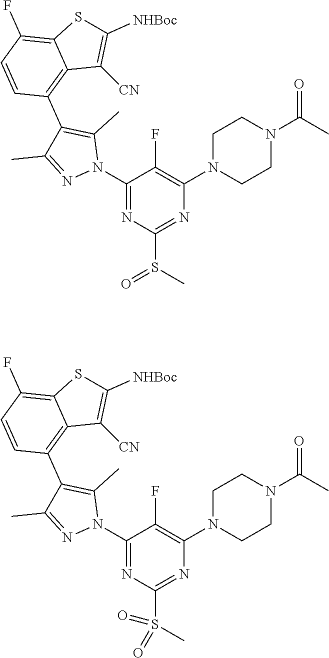 [SMILES: CC(=O)N1CCN(c2nc(S(C)=O)nc(-n3nc(C)c(-c4ccc(F)c5sc(NC(=O)OC(C)(C)C)c(C#N)c45)c3C)c2F)CC1 ; CC(=O)N1CCN(c2nc(S(C)(=O)=O)nc(-n3nc(C)c(-c4ccc(F)c5sc(NC(=O)OC(C)(C)C)c(C#N)c45)c3C)c2F)CC1]

mCPBA (7.91 mg, 0.046 mmol) was added to a solution of 4-(1-(6-(4-acetylpiperazin-1-yl)-5-fluoro-2-(methylthio)pyrimidin-4-yl)-3,5-dimethyl-1H-pyrazol-4-yl)-2-amino-7-fluorobenzo[b]thiophene-3-carbonitrile (20 mg, 0.031 mmol) in DCM (1 mL) at 0° C., then continued stirring at 0° C. for 10 min. Then the mixture was concentrated, and the residue was purified by FCC (0˜10% MeOH in DCM) to give a mixture of sulfone and sulfoxide products (>99% combined yield). LC-MS calc. for C₃₀H₃₃F₂N₈O₄S₂ (M+H)⁺: m/z=671.2; found 671.2., and LC-MS calc. for C₃₀H₃₃F₂N₈O₅S₂ (M+H)⁺: m/z=687.2; found 687.2.

### Step 3. 4-(1-(6-(4-Acetylpiperazin-1-yl)-5-fluoro-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile

The mixture from the previous step was dissolved in THF (1 mL), then (S)-1-((S)-1-methylpyrrolidin-2-yl)ethan-1-ol (7.89 mg, 0.061 mmol) was added. The solution was cooled to 0° C., and LHMDS (1 M in THF, 0.061 mL, 0.061 mmol) was added dropwise. The mixture was stirred at 50° C. for 1 h. The volatiles were removed under reduced pressure, then the residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₁H₃₆F₂N₉O₂S (M+H)⁺: m/z=636.3; found 636.3.

Peak 2, diastereomer 2. LC-MS calc. for C₃₁H₃₆F₂N₉O₂S (M+H)⁺: m/z=636.3; found 636.3.* This peak is the more potent one.

Diastereomers of Example 51 are understood to be:
(Rₐ)-4-(1-(6-(4-acetylpiperazin-1-yl)-5-fluoro-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-5-fluoro-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile.

### Example 52. 4-(4-Acetylpiperazin-1-yl)-6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidine-5-carbonitrile

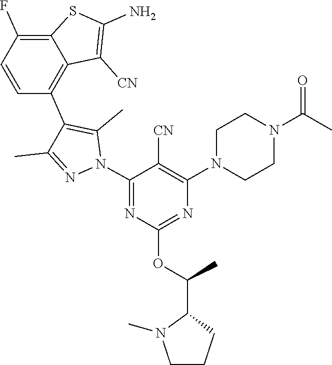 [SMILES: CC(=O)N1CCN(c2nc(O[C@@H](C)[C@@H]3CCCN3C)nc(-n3nc(C)c(-c4ccc(F)c5sc(N)c(C#N)c45)c3C)c2C#N)CC1]

### Step 1. tert-Butyl (4-(1-(6-(4-acetylpiperazin-1-yl)-5-cyano-2-(methylthio)pyrimidin-4-yl)-3,5-dimethyl-1H-pyrazol-4-yl)-3-cyano-7-fluorobenzo[b]thiophene-2-yl)carbamate

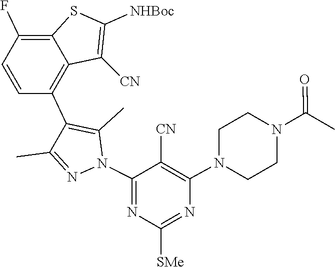 [SMILES: CSc1nc(N2CCN(C(C)=O)CC2)c(C#N)c(-n2nc(C)c(-c3ccc(F)c4sc(NC(=O)OC(C)(C)C)c(C#N)c34)c2C)n1]

Cs₂CO₃ (50.6 mg, 0.155 mmol) was added to a solution of Intermediate 1 (20 mg, 0.052 mmol) and 4,6-dichloro-2-(methylthio)pyrimidine-5-carbonitrile (22.78 mg, 0.104 mmol) in MeCN (1 mL), then the mixture was stirred at r.t. for 1 h. 1-(piperazin-1-yl)ethan-1-one (19.90 mg, 0.155 mmol) was added to the reaction, and the resulting mixture was stirred at 50° C. for 30 min. The insoluble salts were filtered off, and the filtrate was concentrated. The residue was purified by FCC (0˜100% EtOAc in hexanes) to afford the title compound (23 mg, 67% yield). LC-MS calc. for C₃₁H₃₃F₁N₉O₃S₂ (M+H)⁺: m/z=662.2; found 662.2.

### Step 2. tert-Butyl (4-(1-(6-(4-acetylpiperazin-1-yl)-5-cyano-2-(methylsulfinyl)pyrimidin-4-yl)-3,5-dimethyl-1H-pyrazol-4-yl)-3-cyano-7-fluorobenzo[b]thiophene-2-yl)carbamate and tert-butyl (4-(1-(6-(4-acetylpiperazin-1-yl)-5-cyano-2-(methylsulfonyl)pyrimidin-4-yl)-3,5-dimethyl-1H-pyrazol-4-yl)-3-cyano-7-fluorobenzo[b]thiophene-2-yl)carbamate

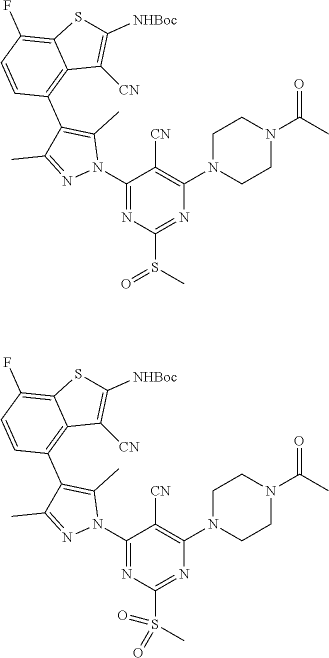 [SMILES: CC(=O)N1CCN(c2nc(S(C)=O)nc(-n3nc(C)c(-c4ccc(F)c5sc(NC(=O)OC(C)(C)C)c(C#N)c45)c3C)c2C#N)CC1 ; CC(=O)N1CCN(c2nc(S(C)(=O)=O)nc(-n3nc(C)c(-c4ccc(F)c5sc(NC(=O)OC(C)(C)C)c(C#N)c45)c3C)c2C#N)CC1]

mCPBA (9.0 mg, 0.052 mmol) was added to a solution of 4-(4-acetylpiperazin-1-yl)-6-(4-(2-amino-3-cyano-7-fluorobenzo[b]thiophene-4-yl)-3,5-dimethyl-1H-pyrazol-1-yl)-2-(methylthio)pyrimidine-5-carbonitrile (23 mg, 0.035 mmol) in DCM (1 mL) at 0° C., stirring was continued at 0° C. for 10 min. Then the mixture was concentrated, and the residue was purified by FCC, (0˜10% MeOH in DCM) to give a mixture of sulfone and sulfoxide product (>99% combined yield). LC-MS calc. for C₃₁H₃₃F₁N₉O₄S₂ (M+H)⁺: m/z=678.2; found 678.2, and LC-MS calc. for C₃₁H₃₃F₁N₉O₅S₂ (M+H)⁺: m/z=694.2; found 694.2.

### Step 3. 4-(4-acetylpiperazin-1-yl)-6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidine-5-carbonitrile

The mixture of products from Step 2 was dissolved in THF (1 mL), then (S)-1-((S)-1-methylpyrrolidin-2-yl)ethan-1-ol (8.98 mg, 0.070 mmol) was added. The solution was cooled to 0° C., and LHMDS (1 M in THF, 0.070 mL, 0.070 mmol) was added dropwise. Then the mixture was stirred at 0° C. for 1 h. Upon completion, the volatiles were removed under reduced pressure, then the residue was dissolved in TFA (1 mL) and stirred at r.t. for 10 min. The mixture was then diluted with MeCN (4 mL) and purified by prep.-LC-MS (XBRIDGE® C₀s column, eluting with a gradient of MeCN/water containing 0.1% TFA, at a flow rate of 60 mL/min.) to afford the title compound. LC-MS calc. for C₃₂H₃₆FN₁₀O₂S (M+H)⁺: m/z=643.3; found 643.3.

Diastereomers of Example 52 are understood to be:
4-(4-acetylpiperazin-1-yl)-6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidine-5-carbonitrile;
4-(4-acetylpiperazin-1-yl)-6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidine-5-carbonitrile.

### Example 53: 2-Amino-4-(1-(4-(4-(cyclopropanecarbonyl)piperazin-1-yl)-6-((S)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile

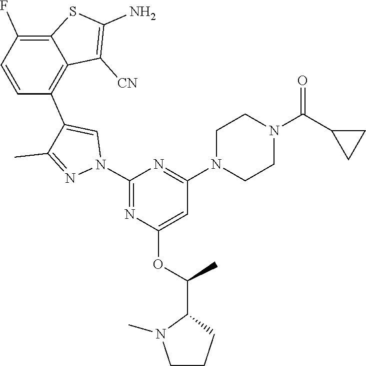 [SMILES: Cc1nn(-c2nc(O[C@@H](C)[C@@H]3CCCN3C)cc(N3CCN(C(=O)C4CC4)CC3)n2)cc1-c1ccc(F)c2sc(N)c(C#N)c12]

### Step 1. tert-Butyl (3-cyano-7-fluoro-4-(3-methyl-1H-pyrazol-4-yl)benzo[b]thiophene-2-yl)carbamate

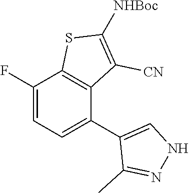 [SMILES: Cc1n[nH]cc1-c1ccc(F)c2sc(NC(=O)OC(C)(C)C)c(C#N)c12]

The title compound was prepared according to the procedure for Intermediate 1, by replacing tert-butyl 3,5-dimethyl-4-(4,4,5,5-tetramethyl-1,3,2-dioxaborolan-2-yl)-1H-pyrazole-1-carboxylate with tert-butyl 3-methyl-4-(4,4,5,5-tetramethyl-1,3,2-dioxaborolan-2-yl)-1H-pyrazole-1-carboxylate. LC-MS calc. for C₁₈H₁₈FN₄O₂S (M+H)⁺: m/z=373.1; found 373.1.

### Step 2. Cyclopropyl(4-(6-((S)-1-((S)-1-methylpyrrolidin-2-yl)ethoxy)-2-(methylsulfonyl)pyrimidin-4-yl)piperazin-1-yl)methanone

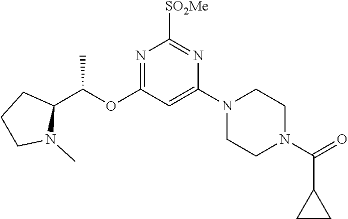 [SMILES: C[C@H](Oc1cc(N2CCN(C(=O)C3CC3)CC2)nc(S(C)(=O)=O)n1)[C@@H]1CCCN1C]

DIPEA (0.340 mL, 1.945 mmol) was added to a solution of Intermediate 2, Step 1 (373 mg, 1.17 mmol) and cyclopropyl(piperazin-1-yl)methanone (150 mg, 0.973 mmol) in MeCN (4 mL), and the resulting mixture was stirred at 80° C. for 1 h. The solution was concentrated, then purified by FCC (0˜20% MeOH in DCM) to give the title compound (280 mg, 66% yield). LC-MS calc. for C₂₀H₃₂N₃₅O₂S (M+H)⁺: m/z=438.2; found 438.2.

### Step 3. 2-Amino-4-(1-(4-(4-(cyclopropanecarbonyl)piperazin-1-yl)-6-((S)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile

Cs₂CO₃ (39.4 mg, 0.121 mmol) was added to a solution of tert-butyl (3-cyano-7-fluoro-4-(3-methyl-1H-pyrazol-4-yl)benzo[b]thiophene-2-yl)carbamate (15 mg, 0.040 mmol) and cyclopropyl(4-(6-((S)-1-((S)-1-methylpyrrolidin-2-yl)ethoxy)-2-(methylsulfonyl)pyrimidin-4-yl)piperazin-1-yl)methanone (26.4 mg, 0.060 mmol) in MeCN (0.5 mL) and the mixture was stirred at 90° C. for 2 h. Upon completion, the volatiles were removed under reduced pressure, then the residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound. LC-MS calc. for C₃₂H₃₇FN₉O₂S (M+H)⁺: m/z=630.3; found 630.3.

Diastereomers of Example 53 are understood to be:
2-amino-(Rₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile.

### Example 54. 2-Amino-7-fluoro-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile

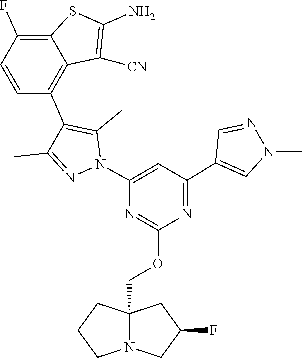 [SMILES: Cc1nn(-c2cc(-c3cnn(C)c3)nc(OC[C@@]34CCCN3C[C@H](F)C4)n2)c(C)c1-c1ccc(F)c2sc(N)c(C#N)c12]

### Step 1. tert-Butyl (4-(1-(6-chloro-2-(methylthio)pyrimidin-4-yl)-3,5-dimethyl-1H-pyrazol-4-yl)-3-cyano-7-fluorobenzo[b]thiophene-2-yl)carbamate

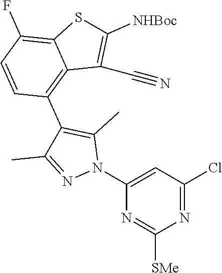 [SMILES: CSc1nc(Cl)cc(-n2nc(C)c(-c3ccc(F)c4sc(NC(=O)OC(C)(C)C)c(C#N)c34)c2C)n1]

Cs₂CO₃ (1265 mg, 3.88 mmol) was added to a solution of Intermediate 1 (500 mg, 1.29 mmol) and 4,6-dichloro-2-(methylthio)pyrimidine (757 mg, 3.88 mmol) in MeCN (12 mL), then the mixture was stirred at 90° C. for 1 h. After cooling to r.t., the insoluble salts were filtered off, and the filtrate was concentrated. The residue was purified by FCC (0˜40% EtOAc in hexanes) to afford the title compound (630 mg, 89% yield). LC-MS calc. for C₂₄H₂₃ClFN₆O₂S₂ (M+H)⁺: m/z=545.1; found 545.1.

### Step 2. tert-Butyl (3-cyano-4-(3,5-dimethyl-1-(6-(1-methyl-1H-pyrazol-4-yl)-2-(methylthio)pyrimidin-4-yl)-1H-pyrazol-4-yl)-7-fluorobenzo[b]thiophene-2-yl)carbamate

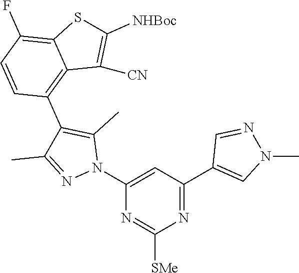 [SMILES: CSc1nc(-c2cnn(C)c2)cc(-n2nc(C)c(-c3ccc(F)c4sc(NC(=O)OC(C)(C)C)c(C#N)c34)c2C)n1]

A mixture of 2-amino-4-(1-(6-chloro-2-(methylthio)pyrimidin-4-yl)-3,5-dimethyl-1H-pyrazol-4-yl)-7-fluorobenzo[b]thiophene-3-carbonitrile (110 mg, 0.202 mmol), 1-methyl-4-(4,4,5,5-tetramethyl-1,3,2-dioxaborolan-2-yl)-1H-pyrazole (126 mg, 0.605 mmol), XPhos Pd G2 (31.8 mg, 0.040 mmol) and K₃PO₄ (129 mg, 0.605 mmol) in 1,4-dioxane (2 mL) and water (0.4 mL) was stirred at 100° C. for 30 min. The mixture was diluted with EtOAc, washed with water and brine, dried over anhydrous Na₂SO₄, and concentrated. The residue was purified by FCC (0˜60% EtOAc in hexanes) to afford the title compound (93 mg, 78% yield). LC-MS calc. for C₂₈H₂₈FN₈O₂S₂ (M+H)⁺: m/z=591.2; found 591.2.

### Step 3. tert-Butyl (3-cyano-4-(3,5-dimethyl-1-(6-(1-methyl-1H-pyrazol-4-yl)-2-(methylsulfinyl)pyrimidin-4-yl)-1H-pyrazol-4-yl)-7-fluorobenzo[b]thiophene-2-yl)carbamate and tert-butyl (3-cyano-4-(3,5-dimethyl-1-(6-(1-methyl-1H-pyrazol-4-yl)-2-(methylsulfonyl)pyrimidin-4-yl)-1H-pyrazol-4-yl)-7-fluorobenzo[b]thiophene-2-yl)carbamate

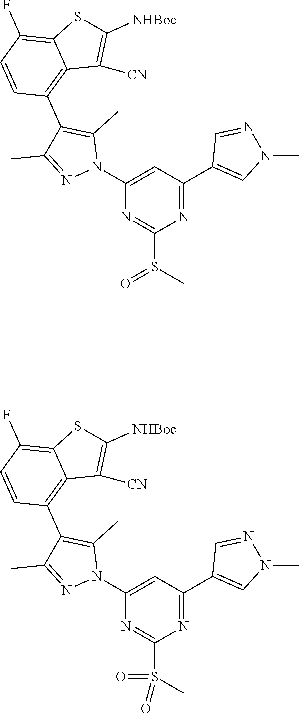 [SMILES: Cc1nn(-c2cc(-c3cnn(C)c3)nc(S(C)=O)n2)c(C)c1-c1ccc(F)c2sc(NC(=O)OC(C)(C)C)c(C#N)c12 ; Cc1nn(-c2cc(-c3cnn(C)c3)nc(S(C)(=O)=O)n2)c(C)c1-c1ccc(F)c2sc(NC(=O)OC(C)(C)C)c(C#N)c12]

mCPBA (40.8 mg, 0.236 mmol) was added to a solution of 2-amino-4-(3,5-dimethyl-1-(6-(1-methyl-1H-pyrazol-4-yl)-2-(methylthio)pyrimidin-4-yl)-1H-pyrazol-4-yl)-7-fluorobenzo[b]thiophene-3-carbonitrile (93 mg, 0.157 mmol) in DCM (8 mL) at 0° C., then continued stirring at 0° C. for 10 min. Then the mixture was concentrated, and the residue was purified by FCC (0˜10% MeOH in DCM) to provide a mixture of the corresponding sulfone and sulfoxide (>99% combined yield C₂₈H₂₈FN₈O₃S₂ (M+H)⁺: m/z=607.2; found 607.2, and LC-MS calc. for C₂₈H₂₈FN₈O₄S₂ (M+H)⁺: m/z=623.2; found 623.2.

### Step 4. 2-Amino-7-fluoro-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile

The mixture from the previous step was dissolved in THF (4 mL), then ((2R,7aS)-2-fluorotetrahydro-1H-pyrrolizin-7a(5H)-yl)methanol (75 mg, 0.471 mmol) was added. The solution was cooled to 0° C., and LHMDS (1 M in THF, 0.471 mL, 0.471 mmol) was added dropwise. Then the mixture was continued stirring at 0° C. for 1 h with LCMS monitoring. Upon completion, the volatiles were removed under reduced pressure. The residue was dissolved in TFA (1 mL) and stirred at r.t. for 10 min. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₀H₃₀F₂N₉OS (M+H)⁺: m/z=602.2; found 602.2. * This peak is the more potent one.

Peak 2, diastereomer 2. LC-MS calc. for C30H30F2N₉OS (M+H)⁺: m/z=602.2; found 602.2.

Diastereomers of Example 54 are understood to be:
2-amino-7-fluoro-(Rₐ)-4-(1-(2-(((SR,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Sₐ)-4-(1-(2-(((SR,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile.

### Example 55. 1-(2-(4-(2-Amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide

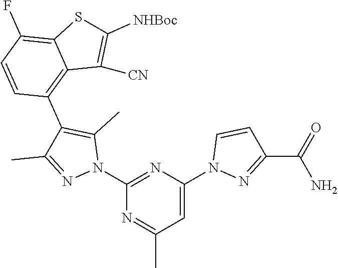 [SMILES: Cc1cc(-n2ccc(C(N)=O)n2)nc(-n2nc(C)c(-c3ccc(F)c4sc(NC(=O)OC(C)(C)C)c(C#N)c34)c2C)n1]

### Step 1. 4-Chloro-6-methyl-2-(methylsulfinyl)pyrimidine

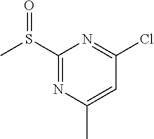

To a solution of 4-chloro-6-methyl-2-(methylthio)pyrimidine (0.529 g, 3.03 mmol) in DCM (6.06 mL) at 0° C. was added 3-chloroperoxybenzoic acid (0.784 g, 4.54 mmol) in small increments. The mixture was allowed to stir for 5 min. at 0° C. before warming to r.t. and stirring for 0.5 h. The mixture was then diluted with water and extracted with dichloromethane (3×). The combined organic layers were dried over Na₂SO₄, filtered and concentrated. The crude was purified by FCC (0-40% EtOAc/hexanes) to afford the title compound as a light yellow oil (>99%). LC-MS calc. for C₆H₈ClN₂OS (M+H)⁺: m/z=191.0; found 191.1.

### Step 2. tert-Butyl (4-(1-(4-chloro-6-methylpyrimidin-2-yl)-3,5-dimethyl-1H-pyrazol-4-yl)-3-cyano-7-fluorobenzo[b]thiophene-2-yl)carbamate

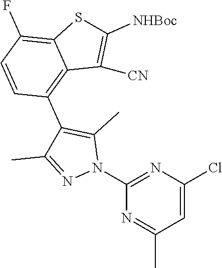 [SMILES: Cc1cc(Cl)nc(-n2nc(C)c(-c3ccc(F)c4sc(NC(=O)OC(C)(C)C)c(C#N)c34)c2C)n1]

In a 40 mL vial was added Intermediate 1 (100 mg, 0.259 mmol), Cs₂CO₃ (253 mg, 0.77 mmol) and 4-chloro-6-methyl-2-(methylsulfonyl)pyrimidine (80 mg, 0.388 mmol). MeCN (1.7 mL) was added and the mixture was stirred for 2 h at r.t. The solids were then removed by filtration and washed with EtOAc. The filtrate was washed with water and brine, dried over Na₂SO₄, filtered and concentrated. The crude was purified by FCC (0-100% EtOAc/hexanes) to afford the title compound as a brown solid. LC-MS calc. for C₂₄H₂₃ClFN₆O₂s (M+H)⁺: m/z=513.1; found 513.1.

### Step 3. 1-(2-(4-(2-Amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide

To tert-butyl (4-(1-(4-chloro-6-methylpyrimidin-2-yl)-3,5-dimethyl-1H-pyrazol-4-yl)-3-cyano-7-fluorobenzo[b]thiophene-2-yl)carbamate (15 mg, 0.029 mmol) in 1,4-dioxane (0.5 mL) was added Cs₂CO₃ (28.6 mg, 0.088 mmol). The mixture was stirred for 3 min. and 1H-pyrazole-3-carboxamide (9.75 mg, 0.088 mmol) was added. The mixture was then stirred at 90° C. for 1.5 h. Once completed, the mixture was diluted with EtOAc and washed with brine. The organic layer was dried over Na₂SO₄, filtered and concentrated. To the residue dissolved in dichloromethane (0.5 mL) was added TFA (0.5 mL). The mixture was stirred at r.t. for 0.5 h. Upon completion, volatiles were removed under reduced pressure and the residue was dissolved in MeCN (4 mL) and water (1 mL) and purified by prep.-LC-MS (XBRIDGE® C₁₈ column, eluting with a gradient of MeCN/water containing 0.1% TFA, at a flow rate of 60 mL/min.) to afford the title compound. LC-MS calc. for C₂₃H₁₉FN₉OS (M+H)⁺: m/z=488.1; found 488.2.

Diastereomers of Example 55 are understood to be:
1-(2-((Rₐ)-4-(2-amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide; and
1-(2-((Sₐ)-4-(2-amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide.

### Example 56: 2-Amino-4-(1-(2-(4-(cyclopropanecarbonyl)piperazin-1-yl)-6-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile

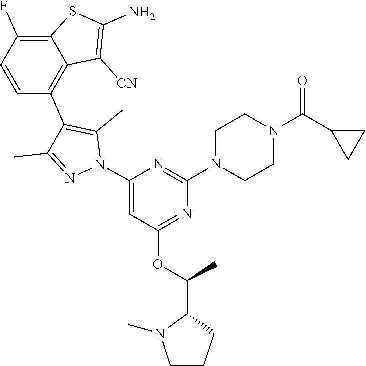 [SMILES: Cc1nn(-c2cc(O[C@@H](C)[C@@H]3CCCN3C)nc(N3CCN(C(=O)C4CC4)CC3)n2)c(C)c1-c1ccc(F)c2sc(N)c(C#N)c12]

### Step 1. (4-(4-chloro-6-((S)-1-((S)-1-methylpyrrolidin-2-yl)ethoxy)pyrimidin-2-yl)piperazin-1-yl)(cyclopropyl)methanone

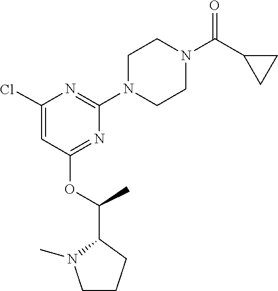 [SMILES: C[C@H](Oc1cc(Cl)nc(N2CCN(C(=O)C3CC3)CC2)n1)[C@@H]1CCCN1C]

DIPEA (0.34 mL, 1.94 mmol) was added to a solution of 4-chloro-6-((S)-1-((S)-1-methylpyrrolidin-2-yl)ethoxy)-2-(methylsulfonyl)pyrimidine (Intermediate 2 Step 1, 373 mg, 1.17 mmol) and cyclopropyl(piperazin-1-yl)methanone (150 mg, 0.97 mmol) in MeCN (4 mL) at r.t., then the mixture was stirred at 80° C. for 1 h. The solution was concentrated in vacuo, then purified by FCC, (0˜10% MeOH in DCM), to give the title compound (100 mg, 26% yield). LC-MS calc. for C₁₉H₂₉ClN₅O₂(M+H)⁺: m/z=394.2; found 394.2.

### Step 2. 2-Amino-4-(1-(2-(4-(cyclopropanecarbonyl)piperazin-1-yl)-6-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile

Cs₂CO₃ (64.5 mg, 0.20 mmol) was added to a solution of Intermediate 1 (38.3 mg, 0.10 mmol) and (4-(4-chloro-6-((S)-1-((S)-1-methylpyrrolidin-2-yl)ethoxy)pyrimidin-2-yl)piperazin-1-yl)(cyclopropyl)methanone (26 mg, 0.066 mmol) in MeCN (1 mL) at r.t., then the mixture was stirred at 95° C. overnight. After cooling to r.t., the mixture was concentrated under reduced pressure. The residue was dissolved in TFA (1 mL) and stirred at r.t. for 10 min. to remove the -Boc protecting group. The mixture was then diluted with MeCN (4 mL) and purified by prep.-LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₃H₃₉FN₉O₂S (M+H)⁺: m/z=643.3; found 643.3.

Peak 2, diastereomer 2. LC-MS calc. for C₃₃H₃₉FN₉O₂S (M+H)⁺: m/z=643.3; found 643.3. * This peak is the more potent one.

Diastereomers of Example 56 are understood to be:
2-amino-(Rₐ)-4-(1-(2-(4-(cyclopropanecarbonyl)piperazin-1-yl)-6-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(2-(4-(cyclopropanecarbonyl)piperazin-1-yl)-6-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile.

### Example 57: 1-(2-(4-(2-Amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-(1-methylcyclopropyl)-1H-pyrazole-3-carboxamide

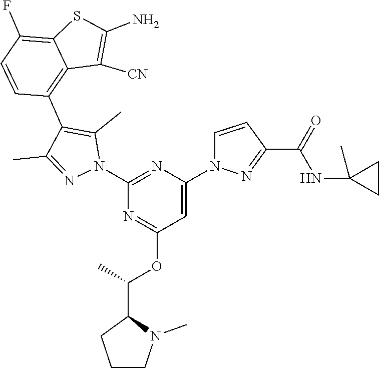 [SMILES: Cc1nn(-c2nc(O[C@@H](C)[C@@H]3CCCN3C)cc(-n3ccc(C(=O)NC4(C)CC4)n3)n2)c(C)c1-c1ccc(F)c2sc(N)c(C#N)c12]

### Step 1. 1-(2-(4-(2-((tert-butoxycarbonyl)amino)-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-1H-pyrazole-3-carboxylic acid

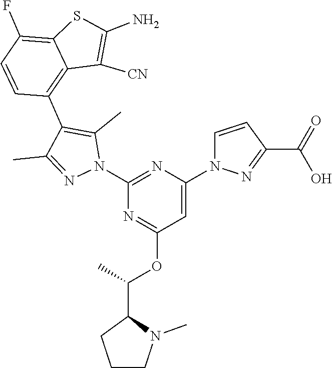 [SMILES: Cc1nn(-c2nc(O[C@@H](C)[C@@H]3CCCN3C)cc(-n3ccc(C(=O)O)n3)n2)c(C)c1-c1ccc(F)c2sc(N)c(C#N)c12]

To a mixture of Intermediate 2 (200 mg, 0.32 mmol), and ethyl 1H-pyrazole-3-carboxylate (134 mg, 0.96 mmol) in 1.4-dioxane (3 mL) was added Cs₂CO₃ (312 mg, 0.96 mmol). The mixture was stirred at 90° C. for 1.5 h. After cooling down to r.t., the solids were filtered over a small pad of celite and the filtrate was concentrated under reduced pressure. The residue was dissolved in MeOH (1.5 mL), followed by addition of 1 M NaOH (1.5 mL), and the resulting mixture was stirred at 60° C. for 1 h. Upon completion, the mixture was acidified by adding 1 M HCl to pH=4 and then diluted and extracted with dichloromethane (5 mL×3). The combined organic phase was dried over Na₂SO₄, filtered and concentrated under reduced pressure to afford the crude carboxylic acid, which was used in the next step without further purification. LC-MS calc. for C₃₄H₃₇FN₉O₅S(M+H)⁺: m/z=702.3; found 702.3.

### Step 2. 1-(2-(4-(2-Amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-(1-methylcyclopropyl)-1H-pyrazole-3-carboxamide

HATU (24.4 mg, 0.064 mmol) was added to a solution of the crude carboxylic acid from previous step (15 mg, 0.021 mmol), DIPEA (11 uL, 0.064 mmol) and 1-methylcyclopropan-1-amine (4.6 mg, 0.064 mmol) in DMF (0.3 mL). The resulting mixture was stirred at r.t. 1 h. Upon completion, the volatiles were removed under reduced pressure. The residue was dissolved in TFA (1 mL) and stirred at r.t. for 10 min. The mixture was then diluted with MeCN (4 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₃H₃₆FN₁₀O₂S (M+H)⁺: m/z=655.3; found 655.4. * This peak is the more potent one.

Peak 2, diastereomer 2. LC-MS calc. for C₃₃H₃₆FN₁₀O₂S (M+H)⁺: m/z=655.3; found 655.4.

Diastereomers of Example 57 are understood to be:
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-(1-methylcyclopropyl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-(1-methylcyclopropyl)-1H-pyrazole-3-carboxamide.

### Example 58: 5-(6-(4-(2-Amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethylpicolinamide

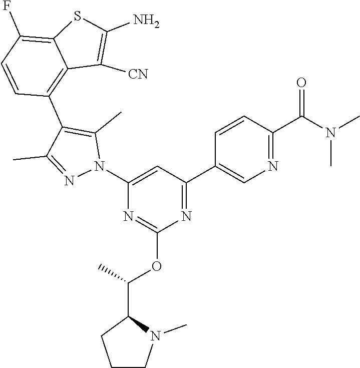 [SMILES: Cc1nn(-c2cc(-c3ccc(C(=O)N(C)C)nc3)nc(O[C@@H](C)[C@@H]3CCCN3C)n2)c(C)c1-c1ccc(F)c2sc(N)c(C#N)c12]

The title compound was prepared according to the procedures described for Example 37, using N,N-dimethyl-5-(4,4,5,5-tetramethyl-1,3,2-dioxaborolan-2-yl)picolinamide as the coupling partner. Two peaks were obtained.

Peak 1, diastereomer 1. LC-MS calc. for C₃₃H₃₅FN₉O₂S (M+H)⁺: m/z=640.3; found 640.4.

Peak 2, diastereomer 2. LC-MS calc. for C₃₃H₃₅FN₉O₂S (M+H)⁺: m/z=640.3; found 640.4. * This peak is the more potent one. ¹H NMR (600 MHz, DMSO) δ 9.96-9.80 (m, 1H, TFA proton), 9.40 (d, J=2.6 Hz, 1H), 8.71 (dd, J=8.2, 2.3 Hz, 1H), 8.25 (s, 1H), 8.04 (s, 2H), 7.76 (d, J=8.2 Hz, 1H), 7.18-7.10 (m, 2H), 5.33 (dq, J=9.4, 6.1 Hz, 1H), 3.86-3.77 (m, 1H), 3.62-3.47 (m, 1H), 3.23-3.11 (m, 1H), 3.16 (s, 3H), 3.04 (d, J=4.9 Hz, 3H), 2.99 (s, 3H), 2.54 (s, 3H), 2.33-2.24 (m, 1H), 2.11 (s, 3H), 2.10-2.04 (m, 1H), 1.98-1.81 (m, 2H), 1.49 (d, J=6.1 Hz, 3H).

Diastereomers of Example 58 are understood to be:
5-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethylpicolinamide;
5-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethylpicolinamide.

### Example 59: 2-Amino-4-(3,5-dimethyl-1-(6-(1-methyl-6-oxo-1,6-dihydropyridin-3-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

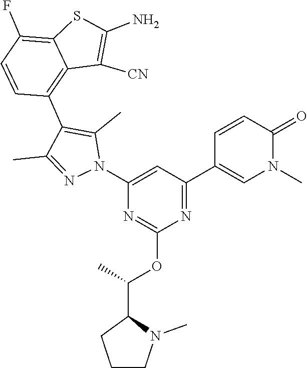 [SMILES: Cc1nn(-c2cc(-c3ccc(=O)n(C)c3)nc(O[C@@H](C)[C@@H]3CCCN3C)n2)c(C)c1-c1ccc(F)c2sc(N)c(C#N)c12]

The title compound was prepared according to the procedures described for Example 37, using 1-methyl-5-(4,4,5,5-tetramethyl-1,3,2-dioxaborolan-2-yl)pyridin-2(1H)-one as the coupling partner. Two peaks were obtained.

Peak 1, diastereomer 1. LC-MS calc. for C₃₁H₃₂FN₈O₂S (M+H)⁺: m/z=599.2; found 599.3.

Peak 2, diastereomer 2. LC-MS calc. for C₃₁H₃₂FN₈O₂S (M+H)⁺: m/z=599.2; found 599.3.* This peak is the more potent one. ¹H NMR (500 MHz, DMSO) δ 9.80 (s, 1H), 8.89 (d, J=2.7 Hz, 1H), 8.27 (dd, J=9.5, 2.7 Hz, 1H), 8.02 (s, 1H), 8.01 (s, 2H), 7.19-7.07 (m, 2H), 6.56 (d, J=9.5 Hz, 1H), 5.26 (dq, J=12.1, 6.1 Hz, 1H), 3.83-3.75 (m, 1H), 3.61 (s, 3H), 3.60-3.51 (m, 1H), 3.21-3.12 (m, 1H), 3.03 (d, J=4.8 Hz, 3H), 2.51 (s, 3H), 2.33-2.23 (m, 1H), 2.13-2.03 (m, 4H), 1.98-1.79 (m, 2H), 1.45 (d, J=6.1 Hz, 3H).

Diastereomers of Example 59 are understood to be:
2-amino-(Rₐ)-4-(3,5-dimethyl-1-(6-(1-methyl-6-oxo-1,6-dihydropyridin-3-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-6-oxo-1,6-dihydropyridin-3-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 60: 4-(6-(4-(2-Amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-2-fluoro-N-methylbenzamide

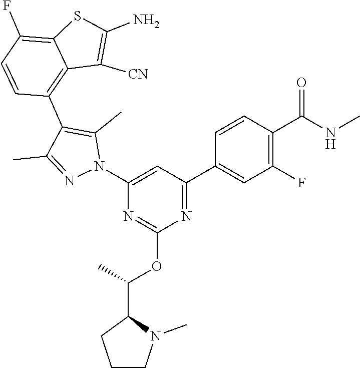 [SMILES: CNC(=O)c1ccc(-c2cc(-n3nc(C)c(-c4ccc(F)c5sc(N)c(C#N)c45)c3C)nc(O[C@@H](C)[C@@H]3CCCN3C)n2)cc1F]

The title compound was prepared according to the procedures described for Example 37, using (3-fluoro-4-(methylcarbamoyl)phenyl)boronic acid as the coupling partner. Two peaks were obtained.

Peak 1, diastereomer 1. LC-MS calc. for C₃₃H₃₃F₂N₈O₂S (M+H)⁺: m/z=643.2; found 643.3.

Peak 2, diastereomer 2. LC-MS calc. for C₃₃H₃₃F₂N₈O₂S (M+H)⁺: m/z=643.2; found 643.3.* This peak is the more potent one. ¹H NMR (500 MHz, DMSO) δ 9.85 (s, 1H), 8.44-8.37 (m, 1H), 8.21 (s, 1H), 8.18-8.10 (m, 2H), 8.03 (s, 2H), 7.82 (t, J=7.8 Hz, 1H), 7.23-7.08 (m, 2H), 5.32 (dq, J=12.0, 5.9 Hz, 1H), 3.86-3.76 (m, 1H), 3.60-3.52 (m, 1H), 3.22-3.12 (m, 1H), 3.05 (d, J=4.9 Hz, 3H), 2.83 (d, J=4.6 Hz, 3H), 2.53 (s, 3H), 2.33-2.22 (m, 1H), 2.14-2.05 (m, 4H), 2.00-1.82 (m, 2H), 1.49 (d, J=6.1 Hz, 3H).

Diastereomers of Example 60 are understood to be:
4-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-2-fluoro-N-methylbenzamide;
4-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-2-fluoro-N-methylbenzamide.

### Examples 61-62

The examples listed in Table 10 were prepared in an analogous fashion to Example 37 using the appropriate coupling partner.

TABLE 10
Examples 61-62

| Ex- | | | |
|---|---|---|---|
| ample | | | ESI |
| # | Structure | IUPAC name | (M + H)⁺ |
| 61 | 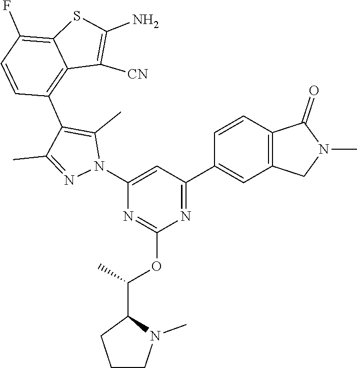 [SMILES: Cc1nn(-c2cc(-c3ccc4c(c3)CN(C)C4=O)nc(O[C@@H](C)[C@@H]3CCCN3C)n2)c(C)c1-c1ccc(F)c2sc(N)c(C#N)c12] | 2-Amino-4-(3,5-dimethyl-1-(6-(2- methyl-1-oxoisoindolin-5-yl)-2- ((S)-1-((S)-1-methylpyrrolidin-2- yl)ethoxy)pyrimidin-4-yl)-1H- pyrazol-4-yl)-7- fluorobenzo[b]thiophene-3- carbonitrile | 637.2 |
| 62 | 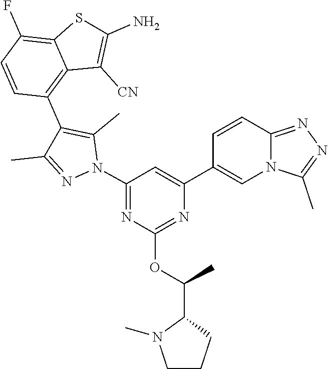 [SMILES: Cc1nn(-c2cc(-c3ccc4nnc(C)n4c3)nc(O[C@@H](C)[C@@H]3CCCN3C)n2)c(C)c1-c1ccc(F)c2sc(N)c(C#N)c12] | 2-Amino-4-(3,5-dimethyl-1-(6-(3- methyl-[1,2,4]triazolo[4,3- a]pyridin-6-yl)-2-((S)-1-((S)-1- methylpyrrolidin-2- yl)ethoxy)pyrimidin-4-yl)-1H- pyrazol-4-yl)-7- fluorobenzo[b]thiophene-3- carbonitrile | 623.2 |

Diastereomers of Examples 61 and 62 are understood to be:

### Example 61

2-amino-(Rₐ)-4-(3,5-dimethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 62

2-amino-(Rₐ)-4-(3,5-dimethyl-1-(6-(3-methyl-[1,2,4]triazolo[4,3-a]pyridin-6-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(3-methyl-[1,2,4]triazolo[4,3-a]pyridin-6-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 63: Methyl 2-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate

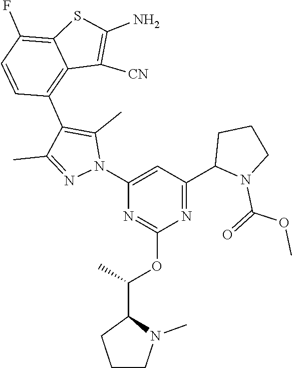 [SMILES: COC(=O)N1CCCC1c1cc(-n2nc(C)c(-c3ccc(F)c4sc(N)c(C#N)c34)c2C)nc(O[C@@H](C)[C@@H]2CCCN2C)n1]

### Step 1. Methyl2-(6-(4-(2-((tert-butoxycarbonyl)amino)-3-cyano-7-fluorobenzo[b]thiophene-4-yl)-3,5-dimethyl-1H-pyrazol-1-yl)-2-(methylthio)pyrimidin-4-yl)pyrrolidine-1-carboxylate

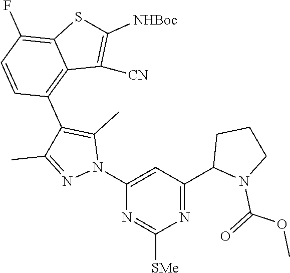 [SMILES: COC(=O)N1CCCC1c1cc(-n2nc(C)c(-c3ccc(F)c4sc(NC(=O)OC(C)(C)C)c(C#N)c34)c2C)nc(SC)n1]

To a 8 mL scintillation vial equipped with a stir bar was added tert-butyl (4-(1-(6-chloro-2-(methylthio)pyrimidin-4-yl)-3,5-dimethyl-1H-pyrazol-4-yl)-3-cyano-7-fluorobenzo[b]thiophene-2-yl)carbamate (Intermediate 3, Step 1, 30 mg, 0.055 mmol), (methoxycarbonyl)proline (19.0 mg, 0.11 mmol), 4,4′-Bis(di-t-butyl)-2,2′-bipyridine (2.95 mg, 0.011 mmol), Cs₂CO₃ (71.7 mg, 0.22 mmol), Nickel(II) chloride ethylene glycol dimethyl ether complex (1.21 mg, 5.50 μmol), and Ir[dF(CF₃)ppy]₂(dtbbpy)PF₆ (0.618 mg, 0.550 μmol). The vial was evacuated and backfilled with N₂ gas, then anhydrous DMA (1.3 mL) was added. The resulting mixture was sparged with N₂ for 5 min. The vial was sealed and placed in front of two Kessil PR160L blue lights (456 nm) and eradiated for 1 h with a cooling fan to maintain a low reaction temperature (<35° C.). Upon completion, the mixture was diluted with EtOAc (5 mL) and washed with saturated NaHCO₃ (2×5 mL), water (2×5 mL), and brine (2×5 mL). The organic layer was dried over Na₂SO₄, filtered and concentrated under reduced pressure. The residue was purified by FCC (eluting with 0˜100% EtOAc in hexanes) to afford the title compound (30 mg, 85% yield). LC-MS calc. for C₃₀H₃₃FN₇O₄S₂ (M+H)⁺: m/z=638.2; found 638.3.

### Step 2. Methyl 2-(6-(4-(2-((tert-butoxycarbonyl)amino)-3-cyano-7-fluorobenzo[b]thiophene-4-yl)-3,5-dimethyl-1H-pyrazol-1-yl)-2-(methylsulfonyl)pyrimidin-4-yl)pyrrolidine-1-carboxylate

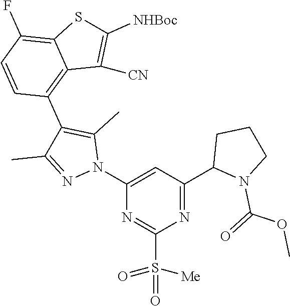 [SMILES: COC(=O)N1CCCC1c1cc(-n2nc(C)c(-c3ccc(F)c4sc(NC(=O)OC(C)(C)C)c(C#N)c34)c2C)nc(S(C)(=O)=O)n1]

mCPBA (20.3 mg, 0.12 mmol) was added to a solution of methyl 2-(6-(4-(2-((tert-butoxycarbonyl)amino)-3-cyano-7-fluorobenzo[b]thiophene-4-yl)-3,5-dimethyl-1H-pyrazol-1-yl)-2-(methylthio)pyrimidin-4-yl)pyrrolidine-1-carboxylate (30 mg, 0.047 mmol) in DCM (1 mL) at 0° C., then the mixture was then stirred at r.t. for 1 h. Upon completion, the mixture was concentrated under reduced pressure, and the residue was purified by FCC (eluting with 0˜10% MeOH in DCM) to give the title compound (30 mg, 98% yield). LC-MS calc. for C₃₀H₃₃FN₇O₆S₂ (M+H)⁺: m/z=670.2; found 670.2.

### Step 3. Methyl 2-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate

LHMDS (1 M in THF, 0.134 mL, 0.134 mmol) was added dropwise to a solution of methyl 2-(6-(4-(2-((tert-butoxycarbonyl)amino)-3-cyano-7-fluorobenzo[b]thiophene-4-yl)-3,5-dimethyl-1H-pyrazol-1-yl)-2-(methylsulfonyl)pyrimidin-4-yl)pyrrolidine-1-carboxylate (30 mg, 0.045 mmol) and (S)-1-((S)-1-methylpyrrolidin-2-yl)ethan-1-ol (0.018 mL, 0.134 mmol) in THF (1 mL) at 0° C., and the resulting mixture was stirred at 0° C. for 1 h. Upon completion, the mixture was concentrated under reduced pressure. The residue was dissolved in TFA (1 mL) and stirred at r.t. for 10 min. The mixture was then diluted with MeCN (4 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Four peaks were obtained.

Peak 1, diastereomer 1. LC-MS calc. for C₃₁H₃₆FN₈O₃S (M+H)⁺: m/z=619.3; found 619.4.

Peak 2, diastereomer 2. LC-MS calc. for C₃₁H₃₆FN₈O₃S (M+H)⁺: m/z=619.3; found 619.4.

Peak 3, diastereomer 3. LC-MS calc. for C₃₁H₃₆FN₈O₃S (M+H)⁺: m/z=619.3; found 619.4.

Peak 4, diastereomer 4. LC-MS calc. for C₃₁H₃₆FN₈O₃S (M+H)⁺: m/z=619.3; found 619.4. * This peak is the most potent diastereomer.

Diastereomers of Example 63 are understood to be:
methyl (S)-2-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate;
methyl (S)-2-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate.

### Example 64: 2-Amino-4-(3,5-dimethyl-1-(2-((S)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile

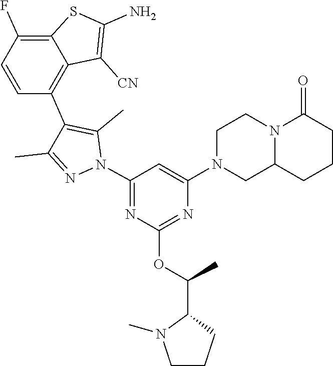 [SMILES: Cc1nn(-c2cc(N3CCN4C(=O)CCCC4C3)nc(O[C@@H](C)[C@@H]3CCCN3C)n2)c(C)c1-c1ccc(F)c2sc(N)c(C#N)c12]

The title compound was prepared according to the procedures described for Example 47, using octahydro-6H-pyrido[1,2-a]pyrazin-6-one hydrochloride as the coupling partner. Two peaks were obtained.

Peak 1, diastereomer 1. LC-MS calc. for C₃₃H₃₉FN₉O₂S(M+H)⁺: m/z=644.3; found 644.4.

Peak 2, diastereomer 2. LC-MS calc. for C₃₃H₃₉FN₉O₂S (M+H)⁺: m/z=644.3; found 644.4. * This peak is the more potent one.

Diastereomers of Example 64 are understood to be:
2-amino-(Rₐ)-4-(3,5-dimethyl-1-(2-((S)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile.

### Example 65. 2-Amino-4-(3,5-dimethyl-1-(6-(6-methyl-5-oxo-2,6-diazaspiro[3.4]octan-2-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

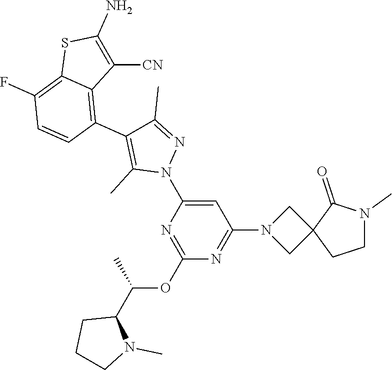 [SMILES: Cc1nn(-c2cc(N3CC4(CCN(C)C4=O)C3)nc(O[C@@H](C)[C@@H]3CCCN3C)n2)c(C)c1-c1ccc(F)c2sc(N)c(C#N)c12]

A mixture of Intermediate 3 (12 mg, 0.019 mmol), 6-methyl-2,6-diazaspiro[3.4]octan-5-one hydrochloride (13.5 mg, 0.077 mmol) and DIPEA (23 μL, 0.13 mmol) in MeCN (192 μL) was stirred at 90° C. for 3 h. The mixture was cooled to r.t., and the volatiles were removed under reduced pressure. The residue was dissolved in TFA (0.4 mL) and stirred at r.t. for 10 min. The mixture was then diluted with MeCN (4.6 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₂H₃₇FN₉O₂S (M+H)⁺: m/z=630.3; found 630.4.

Peak 2, diastereomer 2. LC-MS calc. for C₃₂H₃₇FN₉O₂S (M+H)⁺: m/z=630.3; found 630.4. * This peak is the more potent one.

Diastereomers of Example 65 are understood to be:
2-amino-(Rₐ)-4-(3,5-dimethyl-1-(6-(6-methyl-5-oxo-2,6-diazaspiro[3.4]octan-2-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(6-methyl-5-oxo-2,6-diazaspiro[3.4]octan-2-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 66. 4-(1-(6-((1S,4S)-2-Oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile

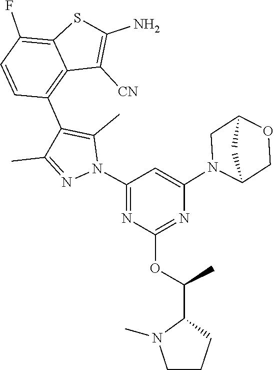 [SMILES: Cc1nn(-c2cc(N3C[C@@H]4C[C@H]3CO4)nc(O[C@@H](C)[C@@H]3CCCN3C)n2)c(C)c1-c1ccc(F)c2sc(N)c(C#N)c12]

The title compound was prepared according to the procedure described for Example 65, using (1 S,4S)-2-oxa-5-azabicyclo[2.2.1]heptane hydrochloride as the nucleophile. Two peaks were obtained.

Peak 1, diastereomer 1. LC-MS calc. for C₃₀H₃₄FN₈O₂S (M+H)⁺: m/z=589.2; found 589.3.

Peak 2, diastereomer 2. LC-MS calc. for C₃₀H₃₄FN₈O₂S (M+H)⁺: m/z=589.2; found 589.3.* This peak is the more potent one.

Diastereomers of Example 66 are understood to be:
(Rₐ)-4-(1-(6-((1 S,4S)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1 S,4S)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile.

### Example 67. 2-Amino-4-(3,5-dimethyl-1-(2-((S)-1-((S)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile

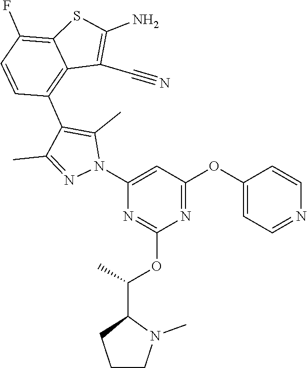 [SMILES: Cc1nn(-c2cc(Oc3ccncc3)nc(O[C@@H](C)[C@@H]3CCCN3C)n2)c(C)c1-c1ccc(F)c2sc(N)c(C#N)c12]

A mixture of Intermediate 3 (20 mg, 0.032 mmol), pyridin-4-ol (15 mg, 0.16 mmol) and K₂CO₃ (22 mg, 0.16 mmol) in DMF (0.2 mL) was stirred at 90° C. for 1 h. The mixture was cooled to r.t., and TFA (1 mL) added and stirred at r.t. for 10 min. The mixture was then diluted with MeCN/water (1:1) to 5 mL and purified by prep. LC-MS (XBRIDGE® C₁₈ column, eluting with a gradient of MeCN/water containing 0.1% TFA, at a flow rate of 60 mL/min.) to afford the title compound as a TFA salt. Two inseparable peaks were obtained.

LC-MS calc. for C₃₀H₃₀FN₈O₂S (M+H)⁺: m/z=585.2; found 585.3.

Diastereomers of Example 67 are understood to be:
2-amino-(Rₐ)-4-(3,5-dimethyl-1-(2-((S)-1-((S)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile.

### Example 68. 2-Amino-7-fluoro-4-(1-(6-((3-fluoropyridin-4-yl)oxy)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile

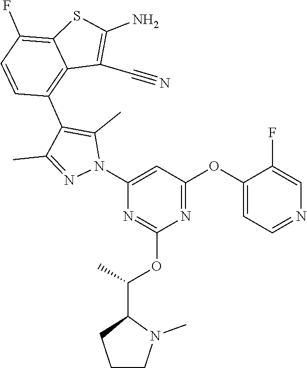 [SMILES: Cc1nn(-c2cc(Oc3ccncc3F)nc(O[C@@H](C)[C@@H]3CCCN3C)n2)c(C)c1-c1ccc(F)c2sc(N)c(C#N)c12]

The title compound was prepared according to the procedure described for Example 67 by replacing pyridin-4-ol with 3-fluoropyridin-4-ol.

Peak 1, diastereomer 1. LC-MS calc. for C₃₀H₂₉F₂N₈O₂S (M+H)⁺: m/z=603.2; found 603.3.

Peak 2, diastereomer 2. LC-MS calc. for C₃₀H₂₉F₂N₈O₂S (M+H)⁺: m/z=603.2; found 603.3. * This peak is the more potent one.

Diastereomers of Example 68 are understood to be:
2-amino-7-fluoro-(Rₐ)-4-(1-(6-((3-fluoropyridin-4-yl)oxy)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-((3-fluoropyridin-4-yl)oxy)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile.

### Example 69. 4-(1-(6-(1-Acetylpiperidin-4-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile

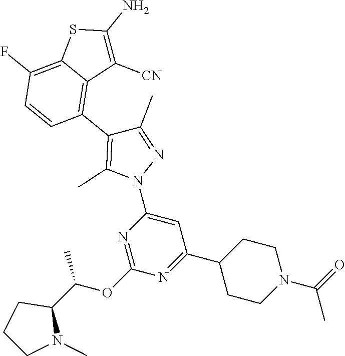 [SMILES: CC(=O)N1CCC(c2cc(-n3nc(C)c(-c4ccc(F)c5sc(N)c(C#N)c45)c3C)nc(O[C@@H](C)[C@@H]3CCCN3C)n2)CC1]

### Step 1. tert-Butyl 4-(6-(4-(2-((tert-butoxycarbonyl)amino)-3-cyano-7-fluorobenzo[b]thiophen-4-yl)-3,5-dimethyl-1H-pyrazol-1-yl)-2-((S)-1-((S)-1-methylpyrrolidin-2-yl)ethoxy)pyrimidin-4-yl)-3,6-dihydropyridine-1(2H)-carboxylate

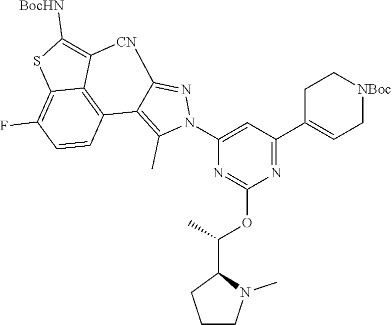

A mixture of Intermediate 3 (60 mg, 0.096 mmol), K₃PO₄ (61 mg, 0.29 mmol), XPhos Pd G2 (15 mg, 0.019 mmol), and tert-butyl 4-(4,4,5,5-tetramethyl-1,3,2-dioxaborolan-2-yl)-3,6-dihydropyridine-1(2H)-carboxylate (89 mg, 0.29 mmol) in 1,4-dioxane (871 μL) and water (87 μL) was bubbled with N₂ gas for 5 min. Then the mixture was stirred at 90° C. for 2 h. Upon completion, the mixture was cooled to r.t. and diluted with EtOAc. The organic layer was washed with water and brine, dried over Na₂SO₄ and concentrated under reduced pressure. The residue was purified by FCC (0-100% EtOAc in hexanes) to provide the title compound (42 mg, 56% yield). LC-MS calc. for C₄₀H₅₀FN₈O₅S (M+H)⁺: m/z=773.4; found 773.5.

### Step 2. 4-(1-(6-(1-Acetylpiperidin-4-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile

Pd/C (10% wt) (18 mg, 0.017 mmol) was added to a mixture of tert-butyl 4-(6-(4-(2-((tert-butoxycarbonyl)amino)-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-3,6-dihydropyridine-1(2H)-carboxylate (18.6 mg, 0.024 mmol) in MeOH (0.6 mL). The mixture was then placed under hydrogen atmosphere (balloon) and stirred at r.t. for 2.5 h. The mixture was filtered through Celite, and the cake was washed with EtOAc. The combined filtrate was concentrated under reduced pressure. The resulting residue was re-dissolved in DCM (0.50 mL) and TFA (0.50 mL), and the resulting mixture was stirred at r.t. for 30 min.

The mixture was concentrated under reduced pressure, and the residue was dissolved in DCM (0.2 mL), followed by addition of DIPEA (0.021 mL, 0.122 mmol). Ac₂0 (2.3 μL, 0.024 mmol) was added to the solution at 0° C., and the mixture was stirred for 10 min. before quenched with MeOH. The volatiles were then removed, and the residue was dissolved in CH₃CN (5.0 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₂H₃₈FN₈O₂S (M+H)⁺: m/z=617.3; found 617.4.

Peak 2, diastereomer 2. LC-MS calc. for C₃₂H₃₈FN₈O₂S (M+H)⁺: m/z=617.3; found 617.4. * This peak is the more potent one.

Diastereomers of Example 69 are understood to be:
(Rₐ)-4-(1-(6-(1-acetylpiperidin-4-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(1-acetylpiperidin-4-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile.

### Example 70: 2-Amino-4-(3,5-dimethyl-1-(4-(1-(methyl-d)-1H-pyrazol-4-yl)-6-((S)-1-((S)-1-methylpyrrolidin-2-yl)ethoxy)pyridin-2-yl)-1H-pyrazol-4-yl)-7-fluorobenzo[b]thiophene-3-carbonitrile

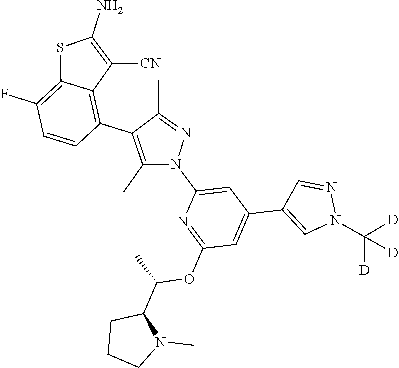 [SMILES: [2H]C([2H])([2H])n1cc(-c2cc(O[C@@H](C)[C@@H]3CCCN3C)nc(-n3nc(C)c(-c4ccc(F)c5sc(N)c(C#N)c45)c3C)c2)cn1]

### Step 1.tert-butyl(3-cyano-7-fluoro-4-(1-(4-iodo-6-((S)-1-((S)-1-methylpyrrolidin-2-yl)ethoxy)pyridin-2-yl)-3,5-dimethyl-1H-pyrazol-4-yl)benzo[b]thiophene-2-yl)carbamate

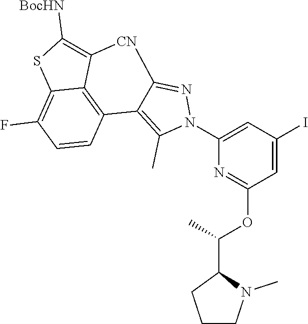

NaH (45.5 mg, 1.14 mmol, 60% wt) was added to a mixture of Intermediate 1 (200 mg, 0.52 mmol) and 2,6-difluoro-4-iodopyridine (150 mg, 0.621 mmol) in DMF (0.60 mL) at 0° C. The resulting mixture was warmed to r.t. and allowed to stir for 1 h before (S)-1-((S)-1-methylpyrrolidin-2-yl)ethan-1-ol (167 mg, 1.29 mmol) and additional NaH (45.5 mg, 1.14 mmol, 60% wt) were added. The mixture was stirred for another 1 h, then quenched with sat. aq. NaHCO₃ at 0° C. The mixture was diluted with EtOAc, washed with water and brine, dried over Na₂SO₄, and concentrated under reduced pressure. The residue was purified by FCC (0-100% EtOAc in hexanes) to provide the title compound (111 mg, 30% yield). LC-MS calc. for C₃₁H₃₅FIN₆O₃S (M+H)⁺: m/z=717.1; found 717.2.

### Step 2. 2-Amino-4-(3,5-dimethyl-1-(4-(1-(methyl-d₃)-1H-pyrazol-4-yl)-6-((S)-1-((S)-1-methylpyrrolidin-2-yl)ethoxy)pyridin-2-yl)-1H-pyrazol-4-yl)-7-fluorobenzo[b]thiophene-3-carbonitrile

A mixture of tert-butyl (3-cyano-7-fluoro-4-(1-(4-iodo-6-((S)-1-((S)-1-methylpyrrolidin-2-yl)ethoxy)pyridin-2-yl)-3,5-dimethyl-1H-pyrazol-4-yl)benzo[b]thiophene-2-yl)carbamate (15 mg, 0.021 mmol), XPhos Pd G2 (3.3 mg, 4.2 μmol), 1-(methyl-d₃)-4-(4,4,5,5-tetramethyl-1,3,2-dioxaborolan-2-yl)-1H-pyrazole (13 mg, 0.063 mmol), and K₃PO₄ (13 mg, 0.063 mmol) in 1,4-dioxane (190 μL) and water (19 μL) was bubbled with N₂ gas for 5 min. Then the mixture was stirred at 90° C. for 1 h. The mixture was cooled to r.t., and the volatiles were removed under reduced pressure. The residue was dissolved in TFA (0.4 mL) and stirred at r.t. for 10 min. The mixture was then diluted with MeCN (4.6 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₀H₂₉D₃FN₈0S (M+H)⁺: m/z=574.3; found 574.3.

Peak 2, diastereomer 2. LC-MS calc. for C₃₀H₂₉D₃FN₈0S (M+H)⁺: m/z=574.3; found 574.3. * This peak is the more potent one.

Diastereomers of Example 70 are understood to be:
2-amino-(Rₐ)-4-(3,5-dimethyl-1-(4-(1-(methyl-d₃)-1H-pyrazol-4-yl)-6-((S)-1-((S)-1-methylpyrrolidin-2-yl)ethoxy)pyridin-2-yl)-1H-pyrazol-4-yl)-7-fluorobenzo[b]thiophene-3-carbonitrile;
2-amino-(Sₐ)-4-(3,5-dimethyl-1-(4-(1-(methyl-d)-1H-pyrazol-4-yl)-6-((S)-1-((S)-1-methylpyrrolidin-2-yl)ethoxy)pyridin-2-yl)-1H-pyrazol-4-yl)-7-fluorobenzo[b]thiophene-3-carbonitrile.

### Example 71. (S)-4-(6-(4-(2-Amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide

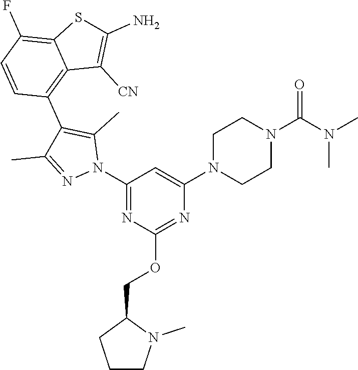 [SMILES: Cc1nn(-c2cc(N3CCN(C(=O)N(C)C)CC3)nc(OC[C@@H]3CCCN3C)n2)c(C)c1-c1ccc(F)c2sc(N)c(C#N)c12]

### Step 1: tert-Butyl (3-cyano-4-(1-(6-(4-(dimethylcarbamoyl)piperazin-1-yl)-2-(methylthio)pyrimidin-4-yl)-3,5-dimethyl-1H-pyrazol-4-yl)-7-fluorobenzo[b]thiophene-2-yl)carbamate

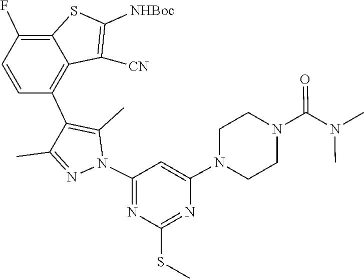 [SMILES: CSc1nc(N2CCN(C(=O)N(C)C)CC2)cc(-n2nc(C)c(-c3ccc(F)c4sc(NC(=O)OC(C)(C)C)c(C#N)c34)c2C)n1]

A mixture of tert-butyl (4-(1-(6-chloro-2-(methylthio)pyrimidin-4-yl)-3,5-dimethyl-1H-pyrazol-4-yl)-3-cyano-7-fluorobenzo[b]thiophene-2-yl)carbamate (Intermediate 3, Step 1, 320 mg, 0.59 mmol), N,N-dimethylpiperazine-1-carboxamide (111 mg, 0.70 mmol), and Cs₂CO₃ (574 mg, 1.76 mmol) was heated at 90° C. for 2 h. The mixture was cooled to r.t., filtered, concentrated and purified over silica gel (gradient of 0-20% MeOH in DCM) to afford the title compound (290 mg, 74% yield). LC-MS calc. for C₃₁H₃₇FN₉O₃S₂ (M+H)*: m/z=666.2; found 666.2.

### Step 2: tert-Butyl (3-cyano-4-(1-(6-(4-(dimethylcarbamoyl)piperazin-1-yl)-2-(methylsulfonyl)pyrimidin-4-yl)-3,5-dimethyl-1H-pyrazol-4-yl)-7-fluorobenzo[b]thiophene-2-yl)carbamate

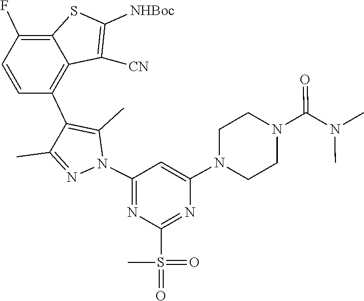 [SMILES: Cc1nn(-c2cc(N3CCN(C(=O)N(C)C)CC3)nc(S(C)(=O)=O)n2)c(C)c1-c1ccc(F)c2sc(NC(=O)OC(C)(C)C)c(C#N)c12]

To a stirred solution of tert-butyl (3-cyano-4-(1-(6-(4-(dimethylcarbamoyl)piperazin-1-yl)-2-(methylthio)pyrimidin-4-yl)-3,5-dimethyl-1H-pyrazol-4-yl)-7-fluorobenzo[b]thiophene-2-yl)carbamate (290 mg, 0.44 mmol) in DCM (29 mL) at 0° C. was added a solution of mCPBA (158 mg, 0.92 mmol) in DCM dropwise. The mixture was then stirred at r.t. for 2 h. The mixture was concentrated, and the residue was purified by FCC (gradient of 0-20% MeOH in DCM) to afford the title compound (285 mg, 94% yield). LC-MS calc. for C₃₁H₃₇FN₉O₅S₂ (M+H)⁺: m/z=698.2; found 698.2.

### Step 3: (S)-4-(6-(4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide

To a stirred solution of tert-butyl (3-cyano-4-(1-(6-(4-(dimethylcarbamoyl)piperazin-1-yl)-2-(methylsulfonyl)pyrimidin-4-yl)-3,5-dimethyl-1H-pyrazol-4-yl)-7-fluorobenzo[b]thiophen-2-yl)carbamate (15 mg, 0.02 mmol) and (S)-(1-methylpyrrolidin-2-yl)methanol (9.90 mg, 0.086 mmol) in THF (0.5 mL) was added LHMDS (1 M in THF, 86 μL, 0.086 mmol) dropwise at 0° C. The mixture was warmed up to r.t. and stirred for an additional 30 min. Upon completion, the mixture was concentrated under reduced pressure, and the residue was dissolved in TFA (1 mL) and stirred at r.t. for 10 min. The mixture was then diluted with MeCN (4 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. LC-MS calc. for C₃₁H₃₈FN₁₀O₂S (M+H)⁺: m/z=633.3; found 633.4.

Enantiomers of Example 71 are understood to be:
(S)-4-(6-((Rₐ)-4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide; and
(S)-4-(6-((Sₐ)-4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide.

### Example 72: (S)-4-(6-(4-(2-Amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide

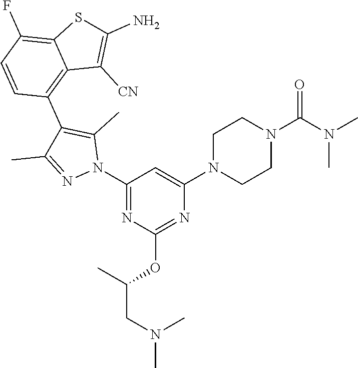 [SMILES: Cc1nn(-c2cc(N3CCN(C(=O)N(C)C)CC3)nc(O[C@@H](C)CN(C)C)n2)c(C)c1-c1ccc(F)c2sc(N)c(C#N)c12]

The title compound was prepared according to the procedure for Example 71 by replacing (S)-(1-methylpyrrolidin-2-yl)methanol with (S)-1-(dimethylamino)propan-2-ol in Step 3. Two peaks were obtained.

Peak 1, diastereomer 1. LC-MS calc. for C₃₀H₃₈FN₁₀O₂S (M+H)⁺: m/z=621.3; found 621.3.

Peak 2, diastereomer 2. LC-MS calc. for C₃₀H₃₈FN₁₀O₂S (M+H)⁺: m/z=621.3; found 621.3.* This peak is the more potent one.

Enantiomers of Example 72 are understood to be:
(S)-4-(6-((Rₐ)-4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide; and
(S)-4-(6-((Sₐ)-4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide.

### Example 73: 2-Amino-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile

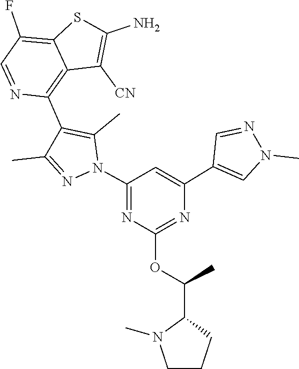 [SMILES: Cc1nn(-c2cc(-c3cnn(C)c3)nc(O[C@@H](C)[C@@H]3CCCN3C)n2)c(C)c1-c1ncc(F)c2sc(N)c(C#N)c12]

### Step 1. tert-Butyl (4-chloro-3-cyano-7-fluorothieno[3,2-c]pyridin-2-yl)carbamate

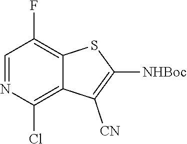 [SMILES: CC(C)(C)OC(=O)Nc1sc2c(F)cnc(Cl)c2c1C#N]

To a solution of 2-amino-4-chloro-7-fluorothieno[3,2-c]pyridine-3-carbonitrile (1.0 g, 4.39 mmol), di-tert-butyl dicarbonate (1438 mg, 6.59 mmol), DMAP (53.7 mg, 0.44 mmol) in DCM (12 mL) and THF (4 mL) was added DIPEA (1.535 mL, 8.79 mmol). The resulting mixture was stirred at 60° C. for 1 h. Upon completion, the mixture was concentrated, then 10 mL hexanes/EtOAc (1/1 ratio) was added. The suspension was stirred at r.t. for 1 h, and the title compound was collected by filtration (1.1 g, 76% yield). LC-MS calc. for C₁₃H₁₂ClFN₃O₂S (M+H)⁺: m/z=328.0; found 328.0.

### Step 2. tert-Butyl (3-cyano-4-(3,5-dimethyl-1H-pyrazol-4-yl)-7-fluorothieno[3,2-c]pyridin-2-yl)carbamate

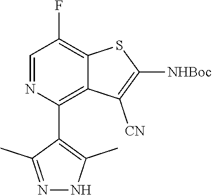 [SMILES: Cc1n[nH]c(C)c1-c1ncc(F)c2sc(NC(=O)OC(C)(C)C)c(C#N)c12]

A mixture of tert-butyl (4-chloro-3-cyano-7-fluorothieno[3,2-c]pyridin-2-yl)carbamate (370 mg, 1.13 mmol), (1-(tert-butoxycarbonyl)-3,5-dimethyl-1H-pyrazol-4-yl)boronic acid (542 mg, 2.26 mmol), SPhos Pd G4 (179 mg, 0.23 mmol), SPhos (185 mg, 0.45 mmol) and K₂CO₃ (468 mg, 3.39 mmol) in 1,4-dioxane (9 mL)/water (2.3 mL) under a N₂ atmosphere was stirred at 100° C. for 3 h. Upon completion, the mixture was filtered through a pad of diatomaceous earth. The filtrate was then concentrated under reduced pressure and purified by FCC (eluting with 0˜100% EtOAc in hexanes) to afford the title compound as white solid (250 mg, 45% yield). LC-MS calc. for C₁₈H₁₉FN₅O₂S (M+H)⁺: m/z=388.1; found 388.2.

### Step 3. tert-Butyl (4-(1-(6-chloro-2-(methylthio)pyrimidin-4-yl)-3,5-dimethyl-1H-pyrazol-4-yl)-3-cyano-7-fluorothieno[3,2-c]pyridin-2-yl)carbamate

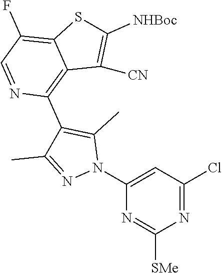 [SMILES: CSc1nc(Cl)cc(-n2nc(C)c(-c3ncc(F)c4sc(NC(=O)OC(C)(C)C)c(C#N)c34)c2C)n1]

To a suspension of tert-butyl (3-cyano-4-(3,5-dimethyl-1H-pyrazol-4-yl)-7-fluorothieno[3,2-c]pyridin-2-yl)carbamate (140 mg, 0.36 mmol), 4,6-dichloro-2-(methylthio)pyrimidine (141 mg, 0.72 mmol) in MeCN (3.6 mL) was added Cs₂CO₃ (235 mg, 0.72 mmol). The mixture was stirred at 90° C. for 2 h. Upon completion, the mixture was cooled to r.t. and filtered through diatomaceous earth washing with EtOAc. The combined filtrate was then concentrated and dissolved in 2 mL DCM. The title compound precipitated from the solution and was collected by filtration (120 mg, 61% yield). LC-MS calc. for C₂₃H₂₂ClFN₇O₂S₂ (M+H)⁺: m/z=546.1; found 546.1.

### Step 4. tert-Butyl (3-cyano-4-(3,5-dimethyl-1-(6-(1-methyl-1H-pyrazol-4-yl)-2-(methylthio)pyrimidin-4-yl)-1H-pyrazol-4-yl)-7-fluorothieno[3,2-c]pyridin-2-yl)carbamate

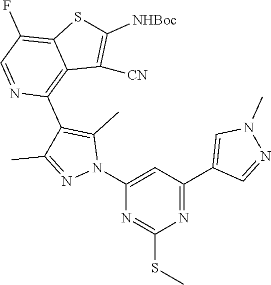 [SMILES: CSc1nc(-c2cnn(C)c2)cc(-n2nc(C)c(-c3ncc(F)c4sc(NC(=O)OC(C)(C)C)c(C#N)c34)c2C)n1]

To a vial charged with tert-butyl (4-(1-(6-chloro-2-(methylthio)pyrimidin-4-yl)-3,5-dimethyl-1H-pyrazol-4-yl)-3-cyano-7-fluorothieno[3,2-c]pyridin-2-yl)carbamate (20 mg, 0.037 mmol), 1-methyl-4-(4,4,5,5-tetramethyl-1,3,2-dioxaborolan-2-yl)-1H-pyrazole (22.9 mg, 0.11 mmol) in 1,4-dioxane (305 μL)/water (61 μL) were added K₃PO₄ (23.3 mg, 0.11 mmol) and XPhos Pd G2 (5.76 mg, 7.33 μmol). The mixture was stirred at 90° C. for 1 h under a N₂ atmosphere. Upon completion, the mixture allowed to cool to r.t., and filtered through a pad of diatomaceous earth. The filtrate was concentrated under reduced pressure and the residue was purified by FCC (eluting with 0˜100% EtOAc in hexanes) to afford the title compound (15 mg, 69% yield). LC-MS calc. for C₂₇H₂₇FN₉O₂S₂ (M+H)⁺: m/z=592.2; found 592.2.

### Step 5. tert-Butyl (3-cyano-4-(3,5-dimethyl-1-(6-(1-methyl-1H-pyrazol-4-yl)-2-(methylthio)pyrimidin-4-yl)-1H-pyrazol-4-yl)-7-fluorothieno[3,2-c]pyridin-2-yl)carbamate

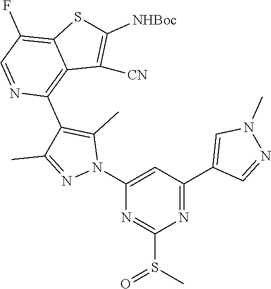 [SMILES: Cc1nn(-c2cc(-c3cnn(C)c3)nc(S(C)=O)n2)c(C)c1-c1ncc(F)c2sc(NC(=O)OC(C)(C)C)c(C#N)c12]

To a solution of tert-butyl (3-cyano-4-(3,5-dimethyl-1-(6-(1-methyl-1H-pyrazol-4-yl)-2-(methylthio)pyrimidin-4-yl)-1H-pyrazol-4-yl)-7-fluorothieno[3,2-c]pyridin-2-yl)carbamate (15 mg, 0.025 mmol) in DCM (500 μL) was added mCPBA (5.25 mg, 0.030 mmol) at 0° C., and the mixture was stirred at 0° C. for 10 min. Then the reaction was quenched by NaHCO₃ (aq) and sodium thiosulfate (aq), and the mixture was extracted with DCM, dried and concentrated to afford the crude product, which was used directly in the next step. LC-MS calc. for C₂₇H₂₇FN₉O₃S₂ (M+H)⁺: m/z=608.2; found 608.3.

### Step 6. 2-Amino-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile

To a solution of the crude product from Step 5 and (S)-1-((S)-1-methylpyrrolidin-2-yl)ethan-1-ol (15.8 mg, 0.12 mmol) in THF (247 μL) at 0° C. was added LHMDS (1 M in THF, 120 μL, 0.12 mmol). The reaction mixture was warmed up to r.t. and stirred for an additional 2 h. The mixture was concentrated under reduced pressure, then the residue was redissolved in 0.5 mL DCM and 0.5 mL TFA and stirred at r.t. for 1 h. Upon completion, the mixture was concentrated, then diluted with MeCN (4 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₈H₃₀FN₁₀OS (M+H)⁺: m/z=573.2; found 573.2.

Peak 2, diastereomer 2. LC-MS calc. for C₂₈H₃₀FN₁₀OS (M+H)⁺: m/z=573.2; found 573.2. * This peak is the more potent one.

Diastereomers of Example 73 are understood to be:
2-amino-(Rₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Sₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile.

### Example 74: 2-Amino-4-(3-chloro-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

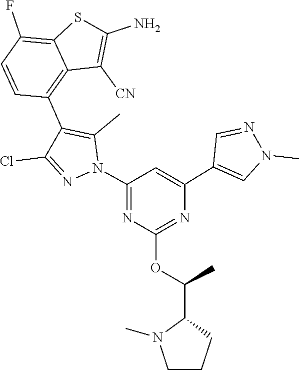 [SMILES: Cc1c(-c2ccc(F)c3sc(N)c(C#N)c23)c(Cl)nn1-c1cc(-c2cnn(C)c2)nc(O[C@@H](C)[C@@H]2CCCN2C)n1]

### Step 1. 4-(4-Bromo-3-chloro-5-methyl-1H-pyrazol-1-yl)-6-(1-methyl-1H-pyrazol-4-yl)-2-(methylthio)pyrimidine

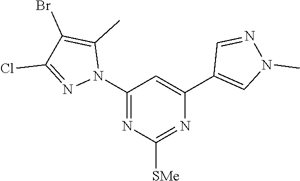 [SMILES: CSc1nc(-c2cnn(C)c2)cc(-n2nc(Cl)c(Br)c2C)n1]

Cs₂CO₃ (541 mg, 1.66 mmol) was added to a solution of 4-bromo-3-chloro-5-methyl-1H-pyrazole (179 mg, 0.91 mmol) and intermediate 5 (200 mg, 0.83 mmol) in CH₃CN (4 mL). The resulting mixture was stirred at 90° C. for 2 h. The mixture was cooled to r.t., and diluted with DCM. The organic phase was washed with water and brine, dried over Na₂SO₄, and concentrated under reduced pressure. The residue was purified by FCC (eluting with 0˜50% EtOAc in hexanes) to afford the title compound (230 mg, 69% yield). LC-MS calc. for C₁₃H₁₃BrClN₆S (M+H)⁺: m/z=399.0; found 399.0.

### Step 2. tert-Butyl (4-(3-chloro-5-methyl-1-(6-(1-methyl-1H-pyrazol-4-yl)-2-(methylthio)pyrimidin-4-yl)-1H-pyrazol-4-yl)-7-fluorobenzo[b]thiophene-2-yl)carbamate

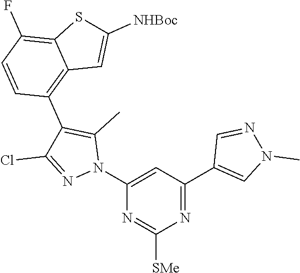 [SMILES: CSc1nc(-c2cnn(C)c2)cc(-n2nc(Cl)c(-c3ccc(F)c4sc(NC(=O)OC(C)(C)C)cc34)c2C)n1]

A mixture of 4-(4-bromo-3-chloro-5-methyl-1H-pyrazol-1-yl)-6-(1-methyl-1H-pyrazol-4-yl)-2-(methylthio)pyrimidine (180 mg, 0.45 mmol), Intermediate 4 (354 mg, 0.90 mmol), PdCl₂(dppf)—CH₂Cl₂ adduct (73.6 mg, 0.09 mmol) and K₂CO₃ (187 mg, 1.35 mmol) in 1,4-dioxane (4 mL) and water (1 mL) was stirred at 100° C. for 5 h. The mixture was cooled to r.t., diluted with EtOAc, washed with water and brine, and dried over Na₂SO₄. The residue after concentration was purified by FCC (eluting with 0˜100% EtOAc in hexanes) to afford the title compound (145 mg, 55% yield). LC-MS calc. for C₂₆H₂₆ClFN₇O₂S₂ (M+H)⁺: m/z=586.1; found 586.1.

### Step 3. tert-Butyl (4-(3-chloro-5-methyl-1-(6-(1-methyl-1H-pyrazol-4-yl)-2-(methylthio)pyrimidin-4-yl)-1H-pyrazol-4-yl)-3-cyano-7-fluorobenzo[b]thiophene-2-yl)carbamate

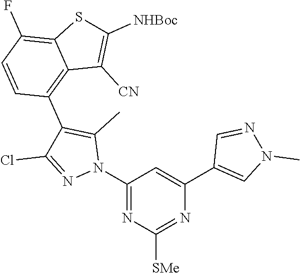 [SMILES: CSc1nc(-c2cnn(C)c2)cc(-n2nc(Cl)c(-c3ccc(F)c4sc(NC(=O)OC(C)(C)C)c(C#N)c34)c2C)n1]

Chlorosulfonyl isocyanate (0.086 mL, 1.0 mmol) was added dropwise to a solution of tert-butyl (4-(3-chloro-5-methyl-1-(6-(1-methyl-1H-pyrazol-4-yl)-2-(methylthio)pyrimidin-4-yl)-1H-pyrazol-4-yl)-7-fluorobenzo[b]thiophene-2-yl)carbamate (145 mg, 0.25 mmol) in MeCN (3 mL) at 0° C. After stirring at 0° C. for 30 min., 1H-indole (87.8 mg, 0.75 mmol) was added to quench the excess amount of chlorosulfonyl isocyanate. After another 30 min., DMF (0.6 mL) was added at 0° C., and the resulting mixture was stirred with LC-MS monitoring. Upon completion, the reaction was quenched with sat. aq. NH₄Cl, then the mixture was diluted with EtOAc. The organic phase was washed with water (3×) and brine, dried over Na₂SO₄, and concentrated under reduced pressure. The residue was purified by FCC (eluting with 0˜100% EtOAc in hexanes) to afford the title compound (62 mg, 41% yield). LC-MS calc. for C₂₇H₂₅ClFN₈O₂S₂ (M+H)⁺: m/z=611.1; found 611.1.

### Step 4. tert-Butyl (4-(3-chloro-5-methyl-1-(6-(1-methyl-1H-pyrazol-4-yl)-2-(methylsulfinyl)pyrimidin-4-yl)-1H-pyrazol-4-yl)-3-cyano-7-fluorobenzo[b]thiophene-2-yl)carbamate and tert-butyl (4-(3-chloro-5-methyl-1-(6-(1-methyl-1H-pyrazol-4-yl)-2-(methylsulfonyl)pyrimidin-4-yl)-1H-pyrazol-4-yl)-3-cyano-7-fluorobenzo[b]thiophene-2-yl)carbamate

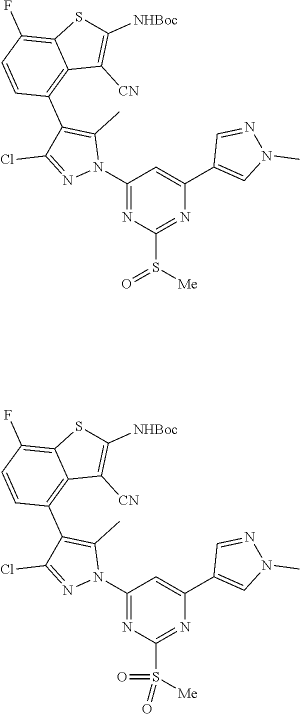 [SMILES: Cc1c(-c2ccc(F)c3sc(NC(=O)OC(C)(C)C)c(C#N)c23)c(Cl)nn1-c1cc(-c2cnn(C)c2)nc(S(C)=O)n1 ; Cc1c(-c2ccc(F)c3sc(NC(=O)OC(C)(C)C)c(C#N)c23)c(Cl)nn1-c1cc(-c2cnn(C)c2)nc(S(C)(=O)=O)n1]

mCPBA (7.91 mg, 0.046 mmol) was added to a solution tert-butyl (4-(3-chloro-5-methyl-1-(6-(1-methyl-1H-pyrazol-4-yl)-2-(methylthio)pyrimidin-4-yl)-1H-pyrazol-4-yl)-3-cyano-7-fluorobenzo[b]thiophene-2-yl)carbamate (60 mg, 0.10 mmol) in DCM (1 mL) at 0° C., then continued stirring at 0° C. for 10 min. Then the mixture was concentrated, and the residue was purified by FCC (0˜10% MeOH in DCM) to give a mixture of sulfone and sulfoxide products (99% combined yield). LC-MS calc. for C₂₇H₂₅ClFN₈O₃S₂ (M+H)⁺: m/z=627.1; found 627.2, and LC-MS calc. for C₂₇H₂₅ClFN₈O₄S₂ (M+H)⁺: m/z=643.1; found 643.1.

### Step 5. 2-amino-4-(3-chloro-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

The mixture from the previous step was dissolved in THF (1 mL), then (S)-1-((S)-1-methylpyrrolidin-2-yl)ethan-1-ol (38.7 mg, 0.30 mmol) was added. The solution was cooled to 0° C., and LHMDS (1 M in THF, 0.3 mL, 0.30 mmol) was added dropwise. The mixture was stirred at 0° C. for 1 h. The volatiles were removed under reduced pressure, then the residue was dissolved in TFA (1 mL) and stirred at r.t. for 10 min. The mixture was then diluted with MeCN (4 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₈H₂₈ClFN₉OS (M+H)⁺: m/z=592.2; found 592.1.

Peak 2, diastereomer 2. LC-MS calc. for C₂₈H₂₈ClFN₉OS (M+H)⁺: m/z=592.2; found 592.1.* This peak is the more potent one. ¹H NMR (500 MHz, DMSO) δ 9.94 (s, 1H), 8.68 (s, 1H), 8.26 (s, 1H), 8.10 (s, 2H), 7.79 (s, 1H), 7.21 (dd, J=8.3, 5.3 Hz, 1H), 7.13 (t, J=8.9 Hz, 1H), 5.25 (dq, J=12.3, 6.1 Hz, 1H), 3.94 (s, 3H), 3.84-3.73 (m, 1H), 3.61-3.52 (m, 1H), 3.21-3.11 (m, 1H), 3.03 (d, J=4.7 Hz, 3H), 2.56 (s, 3H), 2.33-2.24 (m, 1H), 2.14-2.02 (m, 1H), 1.99-1.88 (m, 1H), 1.88-1.76 (m, 1H), 1.45 (d, J=6.1 Hz, 3H).

Diastereomers of Example 74 are understood to be:
2-amino-(Rₐ)-4-(3-chloro-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-chloro-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 75: 2-Amino-7-fluoro-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile

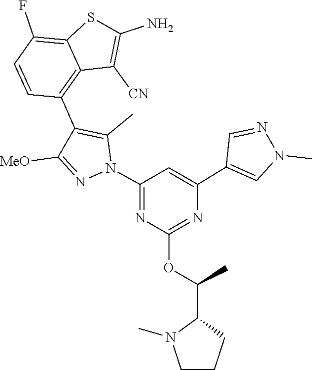 [SMILES: COc1nn(-c2cc(-c3cnn(C)c3)nc(O[C@@H](C)[C@@H]3CCCN3C)n2)c(C)c1-c1ccc(F)c2sc(N)c(C#N)c12]

The title compound was prepared according to the procedure described for Example 74 with modifications. In Step 1, 4-bromo-3-chloro-5-methyl-1H-pyrazole was replaced with 4-bromo-3-methoxy-5-methyl-1H-pyrazole. In Step 2, the catalyst PdCl₂(dppf)—CH₂Cl₂ adduct was replaced with SPhos Pd G4 and Sphos, and the reaction was run at 90° C.

Peak 1, diastereomer 1. LC-MS calc. for C₂₉H₃₁FN₉O₂S (M+H)⁺: m/z=588.2; found 588.2.

Peak 2, diastereomer 2. LC-MS calc, for C₂₉H₃₁FN₉O₂S (M+H)⁺: m/z=588.2; found 588.2. * This peak is the more potent one. ¹H NMR (500 MHz, DMSO) b 9.89 (s, 1H), 8.58 (s, 1H), 8.19 (s, 1H), 8.00 (s, 2H), 7.70 (s, 1H), 7.20-7.10 (m, 1H), 7.12-7.02 (m, 1H), 5.31-5.18 (m, 1H), 3.98 (s, 3H), 3.94 (s, 3H), 3.82-3.70 (m, 1H), 3.62-3.51 (m, 1H), 3.23-3.10 (m, 1H), 3.03 (d, J=4.7 Hz, 3H), 2.56 (s, 3H), 2.34-2.23 (m, 1H), 2.14-2.02 (m, 1H), 2.00-1.88 (m, 1H), 1.89-1.77 (m, 1H), 1.44 (d, J=5.8 Hz, 3H).

Diastereomers of Example 75 are understood to be:
2-amino-7-fluoro-(Rₐ)-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(i-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile.

### Examples 76-77

The following compounds listed in Table 11 were prepared in an analogous fashion to Example 75 using the appropriate pyrazole in Step 1.

TABLE 11
Examples 76-77

| Ex- | | | |
|---|---|---|---|
| ample | | | ESI |
| # | Structure | IUPAC name | [M + H]⁺ |
| 76 | 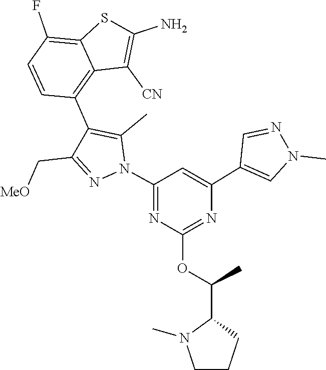 [SMILES: COCc1nn(-c2cc(-c3cnn(C)c3)nc(O[C@@H](C)[C@@H]3CCCN3C)n2)c(C)c1-c1ccc(F)c2sc(N)c(C#N)c12] | 2-Amino-7-fluoro-4-(3-(methoxymethyl)-5- methyl-1-(6-(1-methyl-1H-pyrazol-4-yl)-2- ((S)-1-((S)-1-methylpyrrolidin-2- yl)ethoxy)pyrimidin-4-yl)-1H-pyrazol-4- yl)benzo[b]thiophene-3-carbonitrile | 602.2 |
| 77 | 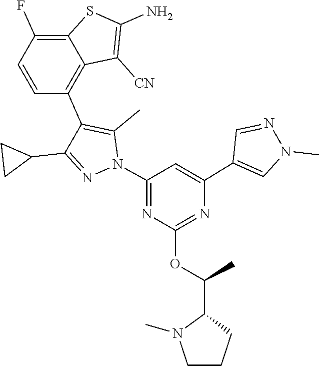 [SMILES: Cc1c(-c2ccc(F)c3sc(N)c(C#N)c23)c(C2CC2)nn1-c1cc(-c2cnn(C)c2)nc(O[C@@H](C)[C@@H]2CCCN2C)n1] | 2-Amino-4-(3-cyclopropyl-5-methyl-1-(6- (1-methyl-1H-pyrazol-4-yl)-2-((S)-1-((S)-1- methylpyrrolidin-2-yl)ethoxy)pyrimidin-4- yl)-1H-pyrazol-4-yl)-7- fluorobenzo[b]thiophene-3-carbonitrile | 598.2 |

Diastereomers of Examples 76 and 77 are understood to be:

### Example 76

2-amino-7-fluoro-(Rₐ)-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile.

### Example 77

2-amino-(Rₐ)-4-(3-cyclopropyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-cyclopropyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 78: 2-Amino-4-(3-(difluoromethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

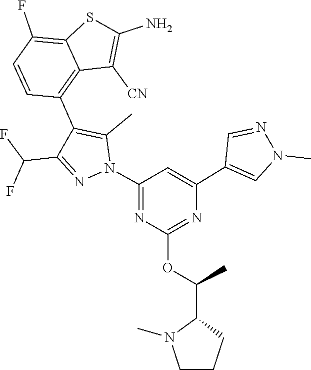 [SMILES: Cc1c(-c2ccc(F)c3sc(N)c(C#N)c23)c(C(F)F)nn1-c1cc(-c2cnn(C)c2)nc(O[C@@H](C)[C@@H]2CCCN2C)n1]

### Step 1. 4-Bromo-3-(difluoromethyl)-5-methyl-1H-pyrazole

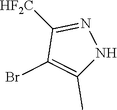

N-Bromosuccinimide (323 mg, 1.82 mmol) was added to a solution of 5-(difluoromethyl)-3-methyl-1H-pyrazole (200 mg, 1.51 mmol) in EtOAc, and the resulting mixture was stirred at 50° C. for 2 h. The mixture was diluted with EtOAc, washed with water and brine subsequently, dried over Na₂SO₄, and concentrated under reduced pressure to give the crude product, which was used without further purifications.

### Step 2. 2-amino-4-(3-(difluoromethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

The title compound was then prepared according to the procedures described for Example 74, by using 4-bromo-3-(difluoromethyl)-5-methyl-1H-pyrazole as the nucleophile in Step 1.

Peak 1, diastereomer 1. LC-MS calc. for C₂₉H₂₉F₃N₉OS (M+H)⁺: m/z=608.2; found 608.2.

Peak 2, diastereomer 2. LC-MS calc. for C₂₉H₂₉F₃N₉OS (M+H)⁺: m/z=608.2; found 608.2. * This peak is the more potent one.

Diastereomers of Example 78 are understood to be:
2-amino-(Rₐ)-4-(3-(difluoromethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-(difluoromethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 79: 2-Amino-4-(5-ethyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

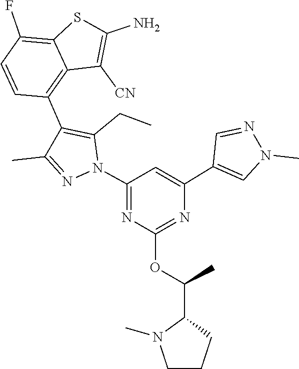 [SMILES: CCc1c(-c2ccc(F)c3sc(N)c(C#N)c23)c(C)nn1-c1cc(-c2cnn(C)c2)nc(O[C@@H](C)[C@@H]2CCCN2C)n1]

### Step 1. 4-(4-Bromo-3-chloro-5-methyl-1H-pyrazol-1-yl)-6-(1-methyl-1H-pyrazol-4-yl)-2-(methylthio)pyrimidine

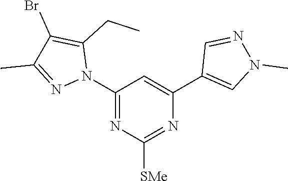

Cs₂CO₃ (1.96 g, 6.0 mmol) was added to a solution of 4-bromo-3-ethyl-5-methyl-1H-pyrazole (380 mg, 2.0 mmol) and Intermediate 5 (581 mg, 2.4 mmol) in CH₃CN (8 mL). The resulting mixture was stirred at 90° C. for 6 h. The mixture was cooled to r.t., then diluted with DCM. The organic phase was washed with water and brine, dried over Na₂SO₄, and concentrated under reduced pressure. The residue was purified by FCC (eluting with 0˜50% EtOAc in hexanes) to afford a mixture of regioisomers. The mixture was further separated by supercritical fluid chromatography (CHIRALPAK® IG column IG5SMO-EN001, Mobile phase: 40% MeOH in CO₂, Flow rate: 50 mL/min. Run time: 11 min.) to afford the title compound (retention time 6.8 min.) as the minor regioisomer (150 mg, 19% yield). LC-MS calc. for C₁₅H₁₈BrN₆S (M+H)⁺: m/z=393.0; found 393.0.

### Step 2. tert-Butyl (3-cyano-4-(5-ethyl-3-methyl-1-(6-(1-methyl-1H-pyrazol-4-yl)-2-(methylthio)pyrimidin-4-yl)-1H-pyrazol-4-yl)-7-fluorobenzo[b]thiophene-2-yl)carbamate

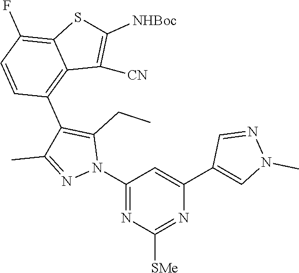 [SMILES: CCc1c(-c2ccc(F)c3sc(NC(=O)OC(C)(C)C)c(C#N)c23)c(C)nn1-c1cc(-c2cnn(C)c2)nc(SC)n1]

A mixture of 4-(4-bromo-5-ethyl-3-methyl-1H-pyrazol-1-yl)-6-(1-methyl-1H-pyrazol-4-yl)-2-(methylthio)pyrimidine (150 mg, 0.38 mmol), tert-butyl (3-cyano-4-(5,5-dimethyl-1,3,2-dioxaborinan-2-yl)-7-fluorobenzo[b]thiophene-2-yl)carbamate (617 mg, 1.53 mmol), SPhos Pd G4 (60.6 mg, 0.076 mmol), SPhos (31.3 mg, 0.076 mmol) and Cs₂CO₃ (621 mg, 1.91 mmol) in DMSO (4 mL) was stirred at 100° C. for 3 h. The mixture was cooled to r.t., diluted with EtOAc, washed with water (3×) and brine, and dried over Na₂SO₄. The residue after concentration was purified by FCC (eluting with 0˜100% EtOAc in hexanes) to afford the title compound (208 mg, 90% yield). LC-MS calc. for C₂₉H₃₀FN₈O₂S₂ (M+H)⁺: m/z=605.2; found 605.2.

### Step 3. tert-Butyl (3-cyano-4-(5-ethyl-3-methyl-1-(6-(1-methyl-1H-pyrazol-4-yl)-2-(methylsulfinyl)pyrimidin-4-yl)-1H-pyrazol-4-yl)-7-fluorobenzo[b]thiophene-2-yl)carbamate and tert-butyl (3-cyano-4-(5-ethyl-3-methyl-1-(6-(1-methyl-1H-pyrazol-4-yl)-2-(methylsulfonyl)pyrimidin-4-yl)-1H-pyrazol-4-yl)-7-fluorobenzo[b]thiophene-2-yl)carbamate

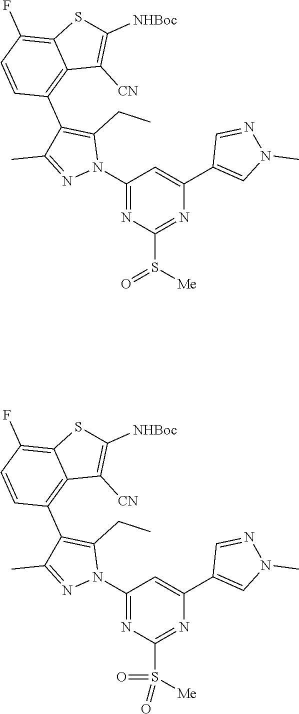 [SMILES: CCc1c(-c2ccc(F)c3sc(NC(=O)OC(C)(C)C)c(C#N)c23)c(C)nn1-c1cc(-c2cnn(C)c2)nc(S(C)=O)n1 ; CCc1c(-c2ccc(F)c3sc(NC(=O)OC(C)(C)C)c(C#N)c23)c(C)nn1-c1cc(-c2cnn(C)c2)nc(S(C)(=O)=O)n1]

mCPBA (89 mg, 0.52 mmol) was added to a solution of tert-butyl (3-cyano-4-(5-ethyl-3-methyl-1-(6-(1-methyl-1H-pyrazol-4-yl)-2-(methylthio)pyrimidin-4-yl)-1H-pyrazol-4-yl)-7-fluorobenzo[b]thiophene-2-yl)carbamate (208 mg, 0.34 mmol) in DCM (4 mL) at 0° C., then stirring at 0° C. was continued stirring for 10 min. The mixture was concentrated under reduced pressure, and the residue was purified by FCC (0˜10% MeOH in DCM) to give a mixture of sulfone and sulfoxide products (99% combined yield). LC-MS calc. for C₂₉H₃₀FN₈O₃S₂ (M+H)⁺: m/z=621.2; found 621.3., and LC-MS calc. for C₂₉H₃₀FN₈O₄S₂ (M+H)⁺: m/z=637.2; found 637.3.

### Step 4. 2-Amino-4-(5-ethyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

The mixture from the previous step was dissolved in THF (6 mL), then (S)-1-((S)-1-methylpyrrolidin-2-yl)ethan-1-ol (133 mg, 1.03 mmol) was added. The solution was cooled to 0° C., and LHMDS (1 M in THF, 1.03 mL, 1.03 mmol) was added dropwise. The mixture was stirred at 0° C. for 1 h. The volatiles were then removed under reduced pressure, then the residue was dissolved in DCM (3 mL), followed by adding TFA (3 mL). After stirring at r.t. for 30 min., the mixture was diluted with MeCN and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₃₀H₃₃FN₉OS (M+H)⁺: m/z=586.2; found 586.3.

Peak 2, diastereomer 2. LC-MS calc. for C₃₀H₃₃FN₉OS (M+H)⁺: m/z=586.2; found 586.3.* This peak is the more potent one. ¹H NMR (500 MHz, DMSO) δ 9.90 (s, 1H), 8.63 (s, 1H), 8.21 (s, 1H), 8.01 (s, 2H), 7.81 (s, 1H), 7.19-7.07 (m, 2H), 5.21 (dq, J=9.9, 6.1 Hz, 1H), 3.94 (s, 3H), 3.84-3.72 (m, 1H), 3.60-3.51 (m, 1H), 3.21-3.09 (m, 2H), 3.04 (d, J=4.8 Hz, 3H), 2.84-2.72 (m, 1H), 2.35-2.24 (m, 1H), 2.15-2.03 (m, 1H), 2.05 (s, 3H), 1.96-1.86 (m, 1H), 1.85-1.76 (m, 1H), 1.46 (d, J=6.1 Hz, 3H), 1.11 (t, J=7.3 Hz, 3H).

Diastereomers of Example 79 are understood to be:
2-amino-(Rₐ)-4-(5-ethyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(5-ethyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile.

### Example 80. 2-Amino-7-fluoro-4-(3-methyl-1-(6-(1-methyl-1H-pyrazol-4-yl)-2-((S)-1-((S)-1-methylpyrrolidin-2-yl)ethoxy)pyrimidin-4-yl)-5-vinyl-1H-pyrazol-4-yl)benzo[b]thiophene-3-carbonitrile

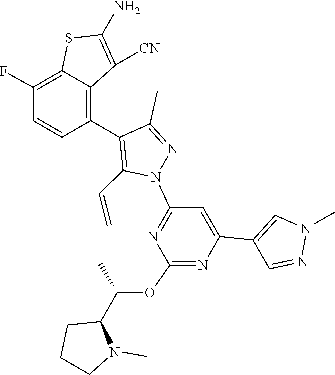 [SMILES: C=Cc1c(-c2ccc(F)c3sc(N)c(C#N)c23)c(C)nn1-c1cc(-c2cnn(C)c2)nc(O[C@@H](C)[C@@H]2CCCN2C)n1]

The title compound was prepared according to the procedure described for Example 79, by replacing tert-butyl (3-cyano-4-(5-ethyl-3-methyl-1-(6-(1-methyl-1H-pyrazol-4-yl)-2-(methylthio)pyrimidin-4-yl)-1H-pyrazol-4-yl)-7-fluorobenzo[b]thiophene-2-yl)carbamate in Step 3 with Intermediate 6b. Two peaks were obtained.

Peak 1, diastereomer 1. LC-MS calc. for C₃₀H₃₁FN₉OS (M+H)⁺: m/z=584.2; found 584.2.

Peak 2, diastereomer 2. LC-MS calc. for C₃₀H₃₁FN₉OS (M+H)⁺: m/z=584.2; found 584.2.* This peak is the more potent one.

Diastereomers of Example 80 are understood to be:
2-amino-7-fluoro-(Rₐ)-4-(3-methyl-1-(6-(1-methyl-1H-pyrazol-4-yl)-2-((S)-1-((S)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(Sₐ)-4-(3-methyl-1-(6-(1-methyl-1H-pyrazol-4-yl)-2-((S)-1-((S)-1-methylpyrrolidin-2-yl)ethoxy)pyrimidin-4-yl)-5-vinyl-1H-pyrazol-4-yl)benzo[b]thiophene-3-carbonitrile.

### Example 81. 2-Amino-7-fluoro-4-(3-(hydroxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile

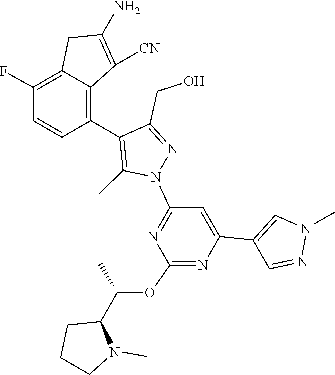 [SMILES: Cc1c(-c2ccc(F)c3c2C(C#N)=C(N)C3)c(CO)nn1-c1cc(-c2cnn(C)c2)nc(O[C@@H](C)[C@@H]2CCCN2C)n1]

### Step 1. tert-Butyl (3-cyano-7-fluoro-4-(5-methyl-1-(6-(1-methyl-1H-pyrazol-4-yl)-2-(methylsulfinyl)pyrimidin-4-yl)-3-vinyl-1H-pyrazol-4-yl)benzo[b]thiophene-2-yl)carbamate

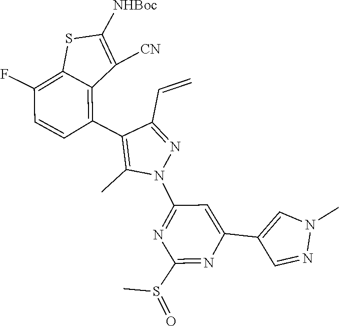 [SMILES: C=Cc1nn(-c2cc(-c3cnn(C)c3)nc(S(C)=O)n2)c(C)c1-c1ccc(F)c2sc(NC(=O)OC(C)(C)C)c(C#N)c12]

To a solution of Intermediate 6a (220 mg, 0.365 mmol) in DCM (4.0 mL) at 0° C. was added mCPBA (90 mg, 0.40 mmol). The resulting mixture was stirred at 0° C. for 15 min. and quenched with sat. aq. Na₂S₂O₃ and diluted with EtOAc. The organic layer was washed with sat. aq. NaHCO₃, and brine. It was then dried over Na₂SO₄, concentrated under reduced pressure and used in the next step without further purification. LC-MS calc. for C₂₉H₂₈FN₈O₃S₂ (M+H)⁺: m/z=619.2; found 619.2.

### Step 2. tert-Butyl (3-cyano-7-fluoro-4-(5-methyl-1-(6-(1-methyl-1H-pyrazol-4-yl)-2-((S)-1-((S)-1-methylpyrrolidin-2-yl)ethoxy)pyrimidin-4-yl)-3-vinyl-1H-pyrazol-4-yl)benzo[b]thiophene-2-yl)carbamate

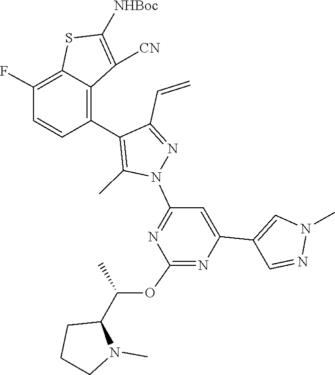 [SMILES: C=Cc1nn(-c2cc(-c3cnn(C)c3)nc(O[C@@H](C)[C@@H]3CCCN3C)n2)c(C)c1-c1ccc(F)c2sc(NC(=O)OC(C)(C)C)c(C#N)c12]

To a solution of (S)-1-((S)-1-methylpyrrolidin-2-yl)ethan-1-ol (92 mg, 0.71 mmol) in THF (1.8 mL) was added dropwise LHMDS (1 M in THF, 710 μL, 0.71 mmol). The resulting mixture was stirred at r.t. for 20 min. before being added dropwise to a solution of tert-butyl (3-cyano-7-fluoro-4-(5-methyl-1-(6-(1-methyl-1H-pyrazol-4-yl)-2-(methylsulfinyl)pyrimidin-4-yl)-3-vinyl-1H-pyrazol-4-yl)benzo[b]thiophene-2-yl)carbamate (220 mg, 0.356 mmol) in THF (1.8 mL). The resulting mixture was stirred at r.t. for 30 min. Upon completion, the. quenched with sat. aq. NH₄Cl and the mixture was extracted with EtOAc (3×). The combine organic phases were dried over Na₂SO₄ and concentrated under reduced pressure. The residue was purified by FCC (0-20% MeOH in DCM) to afford the title compound (120 mg, 49% yield). LC-MS calc. for C₃₅H₃₉FN₉O₃S (M+H)⁺: m/z=684.3; found 684.3.

### Step 3. tert-Butyl (3-cyano-7-fluoro-4-(3-formyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-2-yl)carbamate

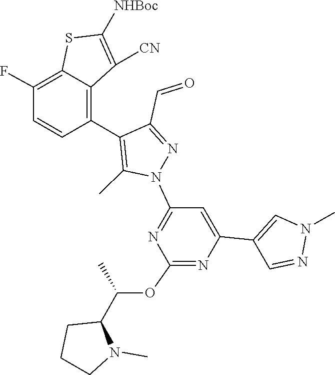 [SMILES: Cc1c(-c2ccc(F)c3sc(NC(=O)OC(C)(C)C)c(C#N)c23)c(C=O)nn1-c1cc(-c2cnn(C)c2)nc(O[C@@H](C)[C@@H]2CCCN2C)n1]

To a solution of tert-butyl (3-cyano-7-fluoro-4-(5-methyl-1-(6-(1-methyl-1H-pyrazol-4-yl)-2-((S)-1-((S)-1-methylpyrrolidin-2-yl)ethoxy)pyrimidin-4-yl)-3-vinyl-1H-pyrazol-4-yl)benzo[b]thiophene-2-yl)carbamate (95 mg, 0.14 mmol) in THF (700 μL) and Water (700 μL) was added OsO₄ (4% in water, 265 μL, 0.042 mmol). After 2 min., NalO₄ (119 mg, 0.556 mmol) was added. The mixture was stirred at r.t. for 30 min. before quenching with sat. aq. Na₂S₂O₃. The mixture was extracted with EtOAc (3×). The combined organic phases were washed with brine, dried over Na₂SO₄ and concentrated under reduced pressure. The residue was purified by FCC (0-20% MeOH in DCM) to afford the title compound (67 mg, 70% yield). LC-MS calc. for C₃₄H₃₇FN₉O₄S (M+H)⁺: m/z=686.3; found 686.4.

### Step 4. 2-Amino-7-fluoro-4-(3-(hydroxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile

To a solution of tert-butyl (3-cyano-7-fluoro-4-(3-formyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-2-yl)carbamate (12 mg, 0.017 mmol) in MeOH (175 μL) was added NaBH₄ (1.33 mg, 0.035 mmol). The resulting mixture was stirred at r.t. for 30 min. then concentrated. To the residue were added water and DCM. The layers were separated, and the aqueous layer was extracted with DCM. The combined organic phases were dried over Na₂SO₄ and concentrated under reduced pressure. The residue was dissolved in TFA (1 mL), and the solution was stirred at r.t. for 10 min. Then the mixture was diluted with MeOH (4 mL) and purified by prep. LC-MS (XBRIDGE® C₁₈ column, eluting with a gradient of MeCN/water containing 0.1% TFA, at a flow rate of 60 mL/min.) to afford the title compound as a TFA salt. Two peaks were obtained.

Peak 1, diastereomer 1. LC-MS calc. for C₂₉H₃₁FN₉O₂S (M+H)⁺: m/z=588.2; found 588.2.

Peak 2, diastereomer 2. LC-MS calc. for C₂₉H₃₁FN₉O₂S (M+H)⁺: m/z=588.2; found 588.2.* This peak is the more potent one.

Diastereomers of Example 81 are understood to be:
2-amino-7-fluoro-(Rₐ)-4-(3-(hydroxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(hydroxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile.

### Example 82. 2-Amino-7-fluoro-4-(5-(hydroxymethyl)-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile

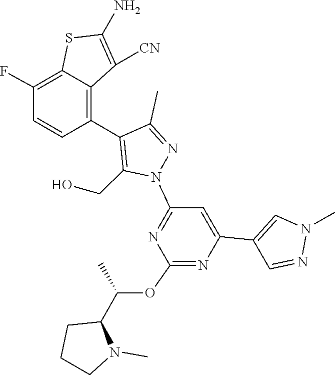 [SMILES: Cc1nn(-c2cc(-c3cnn(C)c3)nc(O[C@@H](C)[C@@H]3CCCN3C)n2)c(CO)c1-c1ccc(F)c2sc(N)c(C#N)c12]

The title compound was prepared according to the procedure described for Example 81, by replacing Intermediate 6a with Intermediate 6b in Step 1. Two peaks were obtained.

Peak 1, diastereomer 1. LC-MS calc. for C₂₉H₃₁FN₉O₂S (M+H)⁺: m/z=588.2; found 588.2. * This peak is the more potent one.

Peak 2, diastereomer 2. LC-MS calc. for C₂₉H₃₁FN₉O₂S (M+H)⁺: m/z=588.2; found 588.2.

Diastereomers of Example 82 are understood to be:
2-amino-7-fluoro-(Rₐ)-4-(5-(hydroxymethyl)-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(5-(hydroxymethyl)-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile.

The compounds in Table 12 table were prepared from Intermediate 7 or Intermediate 8 using General Procedures A and B to provide the specified compound.

### General Procedure A

To a solution of relevant intermediate (0.02 mmol) and relevant alcohol (1 eq.) in THF (0.5 mL) at 0° C. was added LHMDS (1.0 M in THF, 4 eq.) and the resulting mixture stirred at 0° C. for 1 h, then diluted with MeCN (3 mL) and TFA (1 mL). After stirring at r.t. for 2 h, the mixture was filtered and purified by prep. LC-MS (XBRIDGE® C₁₈ column, eluting with a gradient of MeCN/water containing 0.1% TFA, at a flow rate of 60 mL/min.) to afford the product as a TFA salt.

### General Procedure B

To a mixture of relevant intermediate (0.02 mmol), relevant amine (1 eq.) and DIPEA (20 eq.) was added MeCN (0.5 mL) and the mixture heated to 100° C. for 1 h, then diluted with MeCN (3 mL) and TFA (1 mL). After stirring at r.t. for 2 h, the mixture was filtered and purified by prep. LC-MS (XBRIDGE® C₁₈ column, eluting with a gradient of MeCN/water containing 0.1% TFA, at a flow rate of 60 mL/min.) to afford the product as a TFA salt.

TABLE 12
Examples 83-87

| | | | Inter- | Pro- | ESI |
|---|---|---|---|---|---|
| Ex. | Structure | IUPAC name | mediate | cedure | (M + H)⁺ |
| 83 | 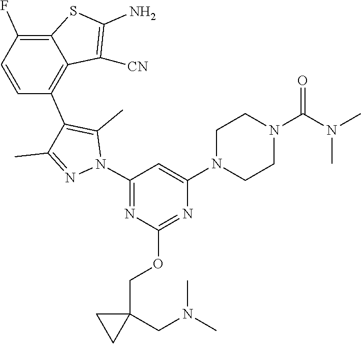 [SMILES: Cc1nn(-c2cc(N3CCN(C(=O)N(C)C)CC3)nc(OCC3(CN(C)C)CC3)n2)c(C)c1-c1ccc(F)c2sc(N)c(C#N)c12] | 4-(6-(4-(2-amino-3-cyano-7- fluorobenzo[b]thiophen-4-yl)-3,5- dimethyl-1H-pyrazol-1- yl)-2-((1-((dimethylamino) methyl)cyclopropyl) methoxy)pyrimidin-4-yl)-N,N- dimethylpiperazine-1- carboxamide | 7 | A | 637.2 |
| 84 | 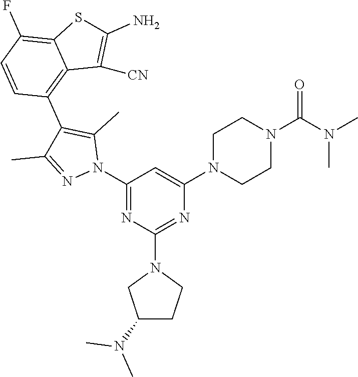 [SMILES: Cc1nn(-c2cc(N3CCN(C(=O)N(C)C)CC3)nc(N3CC[C@H](N(C)C)C3)n2)c(C)c1-c1ccc(F)c2sc(N)c(C#N)c12] | (S)-4-(6-(4-(2-amino-3-cyano-7- fluorobenzo[b]thiophen-4-yl)-3,5- dimethyl-1H-pyrazol-1-yl)-2-(3- (dimethylamino)pyrrolidin-1- yl)pyrimidin-4-yl)-N,N- dimethylpiperazine-1- carboxamide | 7 | B | 632.2 |
| 85 | 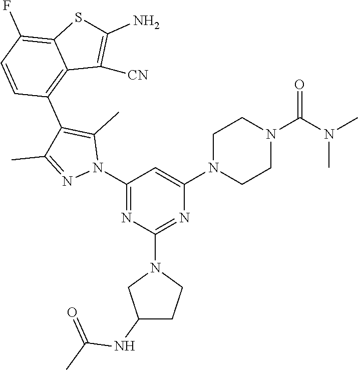 [SMILES: CC(=O)NC1CCN(c2nc(N3CCN(C(=O)N(C)C)CC3)cc(-n3nc(C)c(-c4ccc(F)c5sc(N)c(C#N)c45)c3C)n2)C1] | 4-(2-(3-acetamidopyrrolidin-1-yl)- 6-(4-(2-amino-3-cyano-7- fluorobenzo[b]thiophen-4-yl)-3,5- dimethyl-1H-pyrazol-1- yl)pyrimidin-4-yl)-N,N- dimethylpiperazine-1- carboxamide | 7 | B | 646.3 |
| 86 | 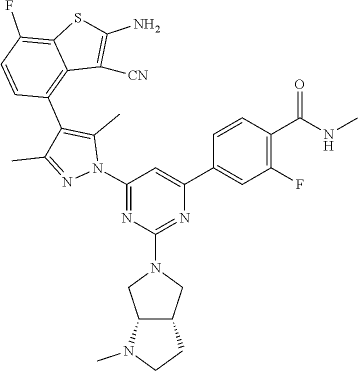 [SMILES: CNC(=O)c1ccc(-c2cc(-n3nc(C)c(-c4ccc(F)c5sc(N)c(C#N)c45)c3C)nc(N3C[C@@H]4CCN(C)[C@@H]4C3)n2)cc1F] | 4-(6-(4-(2-amino-3-cyano-7- fluorobenzo[b]thiophen-4-yl)-3,5- dimethyl-1H-pyrazol-1-yl)-2- ((3aS,6aS)-1- methylhexahydropyrrolo[3,4- b]pyrrol-5(1H)-yl) pyrimidin-4-yl)-2- fluoro-N-methylbenzamide | 8 | B | 640.2 |
| 87 | 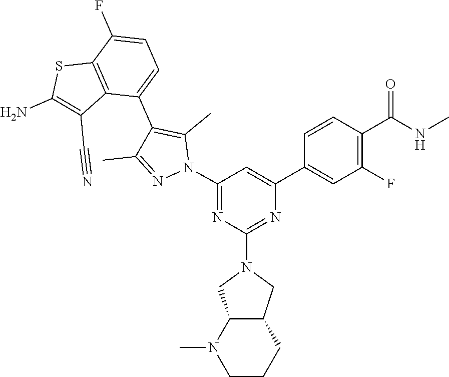 [SMILES: CNC(=O)c1ccc(-c2cc(-n3nc(C)c(-c4ccc(F)c5sc(N)c(C#N)c45)c3C)nc(N3C[C@@H]4CCCN(C)[C@@H]4C3)n2)cc1F] | 4-(6-(4-(2-amino-3-cyano-7- fluorobenzo[b]thiophen-4-yl)-3,5- dimethyl-1H-pyrazol-1-yl)-2- ((4aS,7aS)-1-methyloctahydro- 6H-pyrrolo[3,4-b]pyridin-6- yl)pyrimidin-4-yl)-2-fluoro-N- methylbenzamide | 8 | B | 654.2 |

Diastereomers of the compounds in Table 12 are understood to be:

### Example 83

4-(6-((Rₐ)-4-(2-amino-3-cyano-7-fluorobenzo[b]thiophene-4-yl)-3,5-dimethyl-1H-pyrazol-1-yl)-2-((1-((dimethylamino)methyl)cyclopropyl)methoxy)pyrimidin-4-yl)-N,N-dimethylpiperazine-1-carboxamide; and
4-(6-((Sₐ)-4-(2-amino-3-cyano-7-fluorobenzo[b]thiophene-4-yl)-3,5-dimethyl-1H-pyrazol-1-yl)-2-((1-((dimethylamino)methyl)cyclopropyl)methoxy)pyrimidin-4-yl)-N,N-dimethylpiperazine-1-carboxamide.

### Example 84

(S)-4-(6-((Rₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide;
(S)-4-(6-((Sₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide;
(R)-4-(6-((Rₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide; and
(R)-4-(6-((Sₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide.

### Example 85

4-(2-(3-acetamidopyrrolidin-1-yl)-6-((Rₐ)-4-(2-amino-3-cyano-7-fluorobenzo[b]thiophene-4-yl)-3,5-dimethyl-1H-pyrazol-1-yl)pyrimidin-4-yl)-N,N-dimethylpiperazine-1-carboxamide; and
4-(2-(3-acetamidopyrrolidin-1-yl)-6-((Sₐ)-4-(2-amino-3-cyano-7-fluorobenzo[b]thiophene-4-yl)-3,5-dimethyl-1H-pyrazol-1-yl)pyrimidin-4-yl)-N,N-dimethylpiperazine-1-carboxamide.

### Example 86

4-(6-((Rₐ)-4-(2-amino-3-cyano-7-fluorobenzo[b]thiophene-4-yl)-3,5-dimethyl-1H-pyrazol-1-yl)-2-((3aS,6aS)-1-methylhexahydropyrrolo[3,4-b]pyrrol-5(1H)-yl)pyrimidin-4-yl)-2-fluoro-N-methylbenzamide; and
4-(6-((Sₐ)-4-(2-amino-3-cyano-7-fluorobenzo[b]thiophene-4-yl)-3,5-dimethyl-1H-pyrazol-1-yl)-2-((3aS,6aS)-1-methylhexahydropyrrolo[3,4-b]pyrrol-5(1H)-yl)pyrimidin-4-yl)-2-fluoro-N-methylbenzamide.

### Example 87

4-(6-((Rₐ)-4-(2-amino-3-cyano-7-fluorobenzo[b]thiophene-4-yl)-3,5-dimethyl-1H-pyrazol-1-yl)-2-((4aS,7aS)-1-methyloctahydro-6H-pyrrolo[3,4-b]pyridin-6-yl)pyrimidin-4-yl)-2-fluoro-N-methylbenzamide; and
4-(6-((Sₐ)-4-(2-amino-3-cyano-7-fluorobenzo[b]thiophene-4-yl)-3,5-dimethyl-1H-pyrazol-1-yl)-2-((4aS,7aS)-1-methyloctahydro-6H-pyrrolo[3,4-b]pyridin-6-yl)pyrimidin-4-yl)-2-fluoro-N-methylbenzamide.

### Example 88. 2-Amino-4-(5-ethynyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile

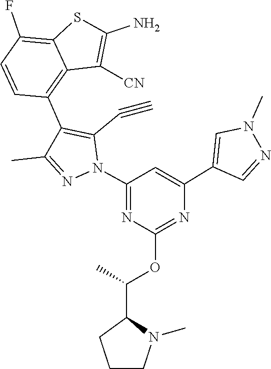 [SMILES: C#Cc1c(-c2ccc(F)c3sc(N)c(C#N)c23)c(C)nn1-c1cc(-c2cnn(C)c2)nc(O[C@@H](C)[C@@H]2CCCN2C)n1]

To a solution of tert-butyl (3-cyano-7-fluoro-4-(3-formyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-2-yl)carbamate (Example 81, Step 3, 10 mg, 0.015 mmol) in MeOH (146 μL) was added NaOtBu (9.8 mg, 0.102 mmol). The mixture was cooled to 0° C. then dimethyl (1-diazo-2-oxopropyl)phosphonate (11 μL, 0.073 mmol) was added. The mixture was stirred at 50° C. for 2 h. then concentrated. The residue was partitioned between water and EtOAc. The layers were separated and the organic layer was dried over magnesium sulfate and concentrated. The crude product was dissolved in TFA (0.5 mL) and stirred at r.t. for 10 min. The mixture was diluted with MeOH and purified by prep. LC-MS (XBRIDGE® C₁₈ column, eluting with a gradient of MeCN/water containing 0.1% NH₄OH, at a flow rate of 60 mL/min.) to afford the title compound. Two peaks were obtained.

Peak 1, diastereomer 1. LC-MS calc. for C₃₀H₂₉FN₉OS (M+H)⁺: m/z=582.2; found 582.2.

Peak 2, diastereomer 2. LC-MS calc. for C₃₀H₂₉FN₉OS (M+H)⁺: m/z=582.2; found 582.2.

Diastereomers of Example 88 are understood to be:
2-amino-4-(5-ethynyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-(Rₐ)-7-fluorobenzo[b]thiophene-3-carbonitrile; and
2-amino-4-(5-ethynyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-(Sₐ)-7-fluorobenzo[b]thiophene-3-carbonitrile.

### Example 89. N-(1-(6-(4-(2-Amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide

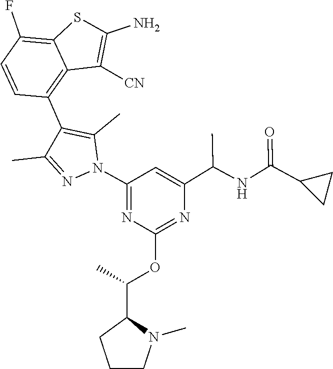 [SMILES: Cc1nn(-c2cc(C(C)NC(=O)C3CC3)nc(O[C@@H](C)[C@@H]3CCCN3C)n2)c(C)c1-c1ccc(F)c2sc(N)c(C#N)c12]

### Step 1. tert-Butyl (4-(1-(6-(1-((tert-butoxycarbonyl)amino)ethyl)-2-(methylthio)pyrimidin-4-yl)-3,5-dimethyl-1H-pyrazol-4-yl)-3-cyano-7-fluorobenzo[b]thiophene-2-yl)carbamate

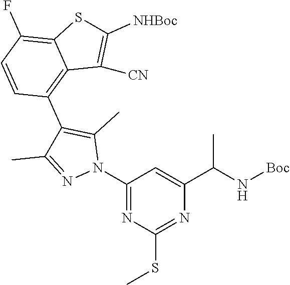 [SMILES: CSc1nc(C(C)NC(=O)OC(C)(C)C)cc(-n2nc(C)c(-c3ccc(F)c4sc(NC(=O)OC(C)(C)C)c(C#N)c34)c2C)n1]

To 8 mL scintillation vial equipped with a stir bar was added tert-butyl (4-(1-(6-chloro-2-(methylthio)pyrimidin-4-yl)-3,5-dimethyl-1H-pyrazol-4-yl)-3-cyano-7-fluorobenzo[b]thiophene-2-yl)carbamate (Intermediate 3, Step 1, 35 mg, 0.064 mmol), N-Boc Alanine (36 mg, 0.19 mmol), 4,4′-Bis(di-t-butyl)-2,2′-bipyridine (3.45 mg, 0.013 mmol), Cs₂CO₃ (84 mg, 0.257 mmol), nickel(II) chloride ethylene glycol dimethyl ether complex (1.411 mg, 6.42 μmol), and Ir[dF(CF₃)ppy]₂(dtbbpy)PF₆ (3.6 mg, 3.2 μmol). The vial was evacuated and an inert atmosphere was established. Anhydrous DMA (1.5 mL) was then added. The mixture was sparged with N₂ for 5 min. The vial was sealed and placed in front of two Kessil PR160L blue lights (456 nm) and eradiated for 2 h with a cooling fan to maintain a low reaction temperature. Upon completion, the mixture was diluted with EtOAc and the layers were separated. The organic layer was washed with water and brine, dried over Na₂SO₄ and concentrated. The crude material was purified by FCC (0-100% EtOAc in hexanes) to provide the title compound. LC-MS calc. for C₃₁H₃₇FN₇O₄S₂ (M+H)⁺: m/z=654.2; found 654.2.

### Step 2. tert-Butyl (4-(1-(6-(1-((tert-butoxycarbonyl)amino)ethyl)-2-(methylsulfonyl)pyrimidin-4-yl)-3,5-dimethyl-1H-pyrazol-4-yl)-3-cyano-7-fluorobenzo[b]thiophene-2-yl)carbamate

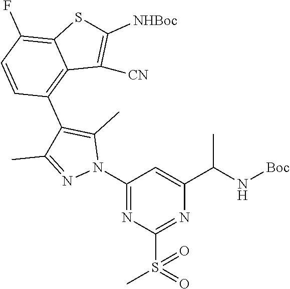 [SMILES: Cc1nn(-c2cc(C(C)NC(=O)OC(C)(C)C)nc(S(C)(=O)=O)n2)c(C)c1-c1ccc(F)c2sc(NC(=O)OC(C)(C)C)c(C#N)c12]

To a solution of the product from the previous step (49 mg, 0.075 mmol) in DCM (400 μL) was added mCPBA (65 mg, 0.38 mmol) and the reaction mixture was stirred at r.t. for 30 min., then diluted with aq. NaHCO₃. The mixture was extracted with DCM and the organic layer was dried over Na₂SO₄ and concentrated. The crude product was purified by FCC (0-100% EtOAc in hexanes) to provide the title compound. LC-MS calc. for C₃₁H₃₇FN₇O₆S₂ (M+H)⁺: m/z=686.2; found 686.2.

### Step 3. tert-Butyl (4-(1-(6-(1-((tert-butoxycarbonyl)amino)ethyl)-2-((S)-1-((S)-1-methylpyrrolidin-2-yl)ethoxy)pyrimidin-4-yl)-3,5-dimethyl-1H-pyrazol-4-yl)-3-cyano-7-fluorobenzo[b]thiophene-2-yl)carbamate

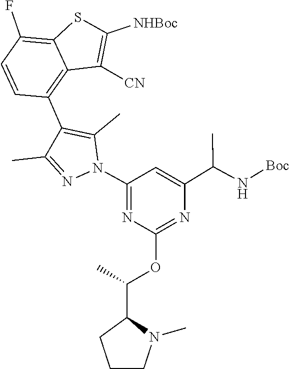 [SMILES: Cc1nn(-c2cc(C(C)NC(=O)OC(C)(C)C)nc(O[C@@H](C)[C@@H]3CCCN3C)n2)c(C)c1-c1ccc(F)c2sc(NC(=O)OC(C)(C)C)c(C#N)c12]

To a solution of the product from the previous step (28 mg, 0.04 mmol) in THF (500 μL) at 0° C. was added (S)-1-((S)-1-methylpyrrolidin-2-yl)ethan-1-ol (11 μL, 0.08 mmol) followed by LHMDS (1.0 M in THF, 82 μL, 0.08 mmol) and the mixture was stirred at 0° C. for 1 h. The reaction was quenched with sat. aq. NH₄Cl and extracted with EtOAc. The organic layer was washed with brine, dried over Na₂SO₄ and concentrated. The crude product was purified by FCC (0-100% EtOAc in hexanes) to provide the title compound. LC-MS calc. for C₃₇H₄₈FN₈O₅S (M+H)⁺: m/z=735.3; found 735.3.

### Step 4. N-(1-(6-(4-(2-Amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide

To a solution of the product from the previous step (30 mg, 0.041 mmol) in DCM (0.5 mL) was added TFA (31 μL, 0.41 mmol) and the mixture was stirred at r.t. for 30 min., then concentrated. To the residue were added cyclopropane carboxylic acid (10 mg, 0.11 mmol), DIPEA (15 mg, 0.11 mmol), HATU (21 mg, 0.056 mmol) and DMF (0.5 mL). The mixture was stirred at r.t. for 30 min., then diluted with MeOH and purified by prep. LC-MS (XBRIDGE® C₁₈ column, eluting with a gradient of MeCN/water containing 0.1% TFA, at a flow rate of 60 mL/min.) to afford the product as a TFA salt. Four isomers were obtained.

Peak 1, diastereomer 1. LC-MS calc. for C₃₁H₃₆FN₈O₂S (M+H)⁺: m/z=603.3; found 603.3.

Peak 2, diastereomer 2. LC-MS calc. for C₃₁H₃₆FN₈O₂S (M+H)⁺: m/z=603.3; found 603.3.

Peak 3, diastereomer 3. LC-MS calc. for C₃₁H₃₆FN₈O₂S (M+H)⁺: m/z=603.3; found 603.3.

Peak 4, diastereomer 4. LC-MS calc. for C₃₁H₃₆FN₈O₂S (M+H)⁺: m/z=603.3; found 603.3.

Diastereomers of Example 89 are understood to be:
N-((R)-1-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide;
N-((R)-1-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide;
N-((S)-1-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide;
N-((S)-1-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide.

### Example 90. 2-Amino-4-(1-(6-((1-(cyclopropanecarbonyl)azetidin-2-yl)methyl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile

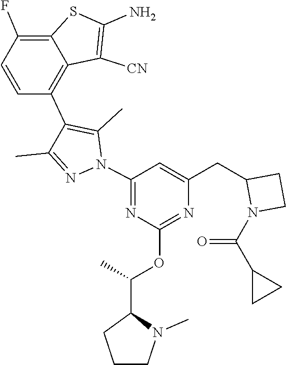 [SMILES: Cc1nn(-c2cc(CC3CCN3C(=O)C3CC3)nc(O[C@@H](C)[C@@H]3CCCN3C)n2)c(C)c1-c1ccc(F)c2sc(N)c(C#N)c12]

The title compound was prepared according to the procedure outlined in Example 89, Steps 1-4, using 2-(1-(tert-butoxycarbonyl)azetidin-2-yl)acetic acid as the coupling partner in Step 1. Two peaks were isolated.

Peak 1, diastereomer 1. LC-MS calc. for C₃₃H₃₈FN₈O₂S (M+H)⁺: m/z=629.3; found 629.3.

Peak 2, diastereomer 2. LC-MS calc. for C₃₃H₃₈FN₈O₂S (M+H)⁺: m/z=629.3; found 629.3.

Diastereomers of Example 90 are understood to be:
2-amino-4-(1-(6-((1-(cyclopropanecarbonyl)azetidin-2-yl)methyl)-2-((S)-1-((S)-1-methylpyrrolidin-2-yl)ethoxy)pyrimidin-4-yl)-3,5-dimethyl-1H-pyrazol-4-yl)-(Rₐ)-7-fluorobenzo[b]thiophene-3-carbonitrile; and
2-amino-4-(1-(6-((1-(cyclopropanecarbonyl)azetidin-2-yl)methyl)-2-((S)-1-((S)-1-methylpyrrolidin-2-yl)ethoxy)pyrimidin-4-yl)-3,5-dimethyl-1H-pyrazol-4-yl)-(Sₐ)-7-fluorobenzo[b]thiophene-3-carbonitrile.

### Example 91. 6-(4-(2-Amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-(1-(pyrimidin-5-yl)ethyl)pyrimidine-4-carboxamide

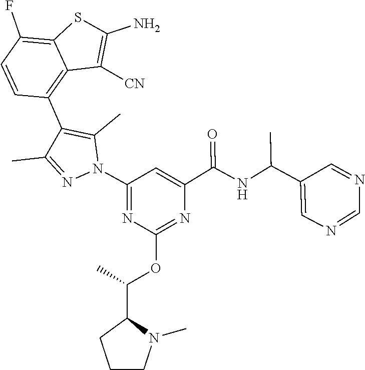 [SMILES: Cc1nn(-c2cc(C(=O)NC(C)c3cncnc3)nc(O[C@@H](C)[C@@H]3CCCN3C)n2)c(C)c1-c1ccc(F)c2sc(N)c(C#N)c12]

### Step 1. 6-Chloro-2-(methylthio)-N-(1-(pyrimidin-5-yl)ethyl)pyrimidine-4-carboxamide

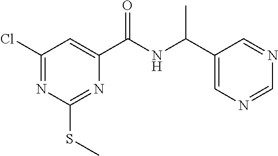 [SMILES: CSc1nc(Cl)cc(C(=O)NC(C)c2cncnc2)n1]

A mixture of 1-(pyrimidin-5-yl)ethan-1-amine (33 mg, 0.27 mmol) and triethylamine (56 μL, 0.403 mmol) in DCM (1.3 mL) was added 6-chloro-2-(methylthio)pyrimidine-4-carbonyl chloride (60 mg, 0.27 mmol) at 0° C., then the mixture was stirred at r.t. for 1 h under N₂. The mixture was diluted with water and extracted with DCM. The organic layer was washed with brine, dried, filtered and concentrated. The residue was purified by FCC (0-100% EtOAc in hexanes) to provide the product. LC-MS calc. for C₁₂H₁₃ClN₅OS (M+H)⁺: m/z=310.1; found 310.1.

### Step 2. tert-Butyl (3-cyano-4-(3,5-dimethyl-1-(2-(methylthio)-6-((1-(pyrimidin-5-yl)ethyl)carbamoyl)pyrimidin-4-yl)-1H-pyrazol-4-yl)-7-fluorobenzo[b]thiophene-2-yl)carbamate

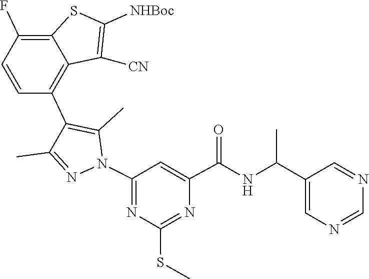 [SMILES: CSc1nc(C(=O)NC(C)c2cncnc2)cc(-n2nc(C)c(-c3ccc(F)c4sc(NC(=O)OC(C)(C)C)c(C#N)c34)c2C)n1]

To Intermediate 1 (82 mg, 0.213 mmol) in MeCN (2 mL) was added 6-chloro-2-(methylthio)-N-(1-(pyrimidin-5-yl)ethyl)pyrimidine-4-carboxamide (66 mg, 0.213 mmol) and Cs₂CO₃ (139 mg, 0.426 mmol). The mixture was stirred at 80° C. for 1 h. After reaction completion, the mixture was cooled to r.t., filtered over a pad of Celite. The solid was washed with EtOAc. The filtrate was concentrated and purified by FCC (0-100% EtOAc in hexanes) to provide the title compound (115 mg, 82% yield). LC-MS calc. for C₃₁H₃₁FN₉O₃S₂ (M+H)⁺: m/z=660.2; found 660.2.

### Step 3. tert-Butyl (3-cyano-4-(3,5-dimethyl-1-(2-(methylsulfonyl)-6-((1-(pyrimidin-5-yl)ethyl)carbamoyl)pyrimidin-4-yl)-1H-pyrazol-4-yl)-7-fluorobenzo[b]thiophene-2-yl)carbamat

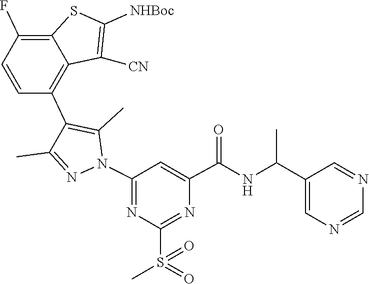 [SMILES: Cc1nn(-c2cc(C(=O)NC(C)c3cncnc3)nc(S(C)(=O)=O)n2)c(C)c1-c1ccc(F)c2sc(NC(=O)OC(C)(C)C)c(C#N)c12]

To a solution of tert-butyl (3-cyano-4-(3,5-dimethyl-1-(2-(methylthio)-6-((1-(pyridin-3-yl)ethyl)carbamoyl)pyrimidin-4-yl)-1H-pyrazol-4-yl)-7-fluorobenzo[b]thiophene-2-yl)carbamate (115 mg, 0.175 mmol) in DCM (1.7 mL) was added mCPBA (36 mg, 0.21 mmol). The mixture was stirred at 0° C. for 15 min. The reaction was quenched with sat. aq. sodium bicarbonate and sat. aq. sodium thiosulfate and extracted with DCM. The organic layer was dried and concentrated. The crude product was used in the next step without further purification. LC-MS calc. for C₃₁H₃₁FN₉O₅S₂ (M+H)⁺: m/z=692.2; found 692.2.

### Step 4. 6-(4-(2-Amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-(1-(pyrimidin-5-yl)ethyl)pyrimidine-4-carboxamide

LHMDS (1.0 M in THF, 260 μL, 0.26 mmol) was added to a solution of tert-butyl (3-cyano-4-(3,5-dimethyl-1-(2-(methylsulfinyl)-6-((1-(pyridin-3-yl)ethyl)carbamoyl)pyrimidin-4-yl)-1H-pyrazol-4-yl)-7-fluorobenzo[b]thiophene-2-yl)carbamate (35 mg, 0.052 mmol) and (S)-1-((S)-1-methylpyrrolidin-2-yl)ethan-1-ol (35 μL, 0.259 mmol) in THF (0.5 mL) at 0° C. After 30 min., the reaction was quenched with TFA (2.0 mL). The reaction was concentrated, then dissolved in TFA (1 mL) and stirred at r.t. for 10 min. The residue was diluted with MeCN and purified by prep. LC-MS (XBRIDGE® C₁₈ column, eluting with a gradient of MeCN/water containing 0.1% TFA, at a flow rate of 60 mL/min.) to afford the product as a TFA salt. Four isomers were obtained.

Peak 1, diastereomer 1. LC-MS calc. for C₃₂H₃₄FN₁₀O₂S (M+H)⁺: m/z=641.2; found 641.2.

Peak 2, diastereomer 2. LC-MS calc. for C₃₂H₃₄FN₁₀O₂S (M+H)⁺: m/z=641.2; found 641.2

Peak 3, diastereomer 3. LC-MS calc. for C₃₂H₃₄FN₁₀O₂S (M+H)⁺: m/z=641.2; found 641.2

Peak 4, diastereomer 4. LC-MS calc. for C₃₂H₃₄FN₁₀O₂S (M+H)⁺: m/z=641.2; found 641.2

Diastereomers of Example 91 are understood to be:
6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-((S)-1-(pyrimidin-5-yl)ethyl)pyrimidine-4-carboxamide;
6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-((S)-1-(pyrimidin-5-yl)ethyl)pyrimidine-4-carboxamide;
6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-((R)-1-(pyrimidin-5-yl)ethyl)pyrimidine-4-carboxamide; and
6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-((R)-1-(pyrimidin-5-yl)ethyl)pyrimidine-4-carboxamide.

### Example 92. 2-Amino-4-(5-ethyl-3-methyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile

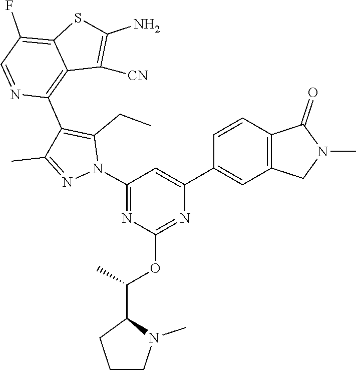 [SMILES: CCc1c(-c2ncc(F)c3sc(N)c(C#N)c23)c(C)nn1-c1cc(-c2ccc3c(c2)CN(C)C3=O)nc(O[C@@H](C)[C@@H]2CCCN2C)n1]

### Step 1. tert-Butyl (3-cyano-4-(5-ethyl-3-methyl-1-(6-(2-methyl-1-oxoisoindolin-5-yl)-2-(methylthio)pyrimidin-4-yl)-1H-pyrazol-4-yl)-7-fluorothieno[3,2-c]pyridin-2-yl)carbamate

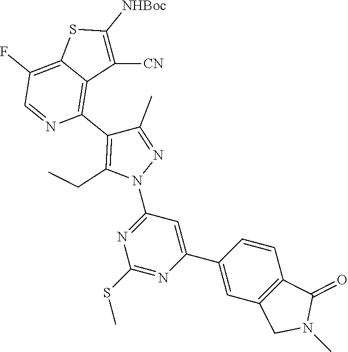 [SMILES: CCc1c(-c2ncc(F)c3sc(NC(=O)OC(C)(C)C)c(C#N)c23)c(C)nn1-c1cc(-c2ccc3c(c2)CN(C)C3=O)nc(SC)n1]

To a 40 mL vial were added Intermediate 10 (300 mg, 0.536 mmol), 2-methyl-5-(4,4,5,5-tetramethyl-1,3,2-dioxaborolan-2-yl)isoindolin-1-one (293 mg, 1.071 mmol), Potassium phosphate (227 mg, 1.071 mmol), and XPhos Pd G2 (84 mg, 0.107 mmol). The vial was then placed under a N₂ atmosphere and 1,4-dioxane (4.5 mL) and water (900 μL) were then added. The reaction was stirred at 100° C. for 1 h. Upon completion, the mixture was cooled down to ambient temperature. The mixture was poured into water and extracted with EtOAc. The organic layer was washed with brine, dried over MgSO₄, and concentrated. The crude product was purified by FCC (0-20% MeOH in DCM) to provide the title compound (280 mg, 78%). LC-MS calc. for C₃₃H₃₂FN₈O₃S₂ (M+H)⁺: m/z=671.2; found 671.2.

### Step 2. tert-Butyl (3-cyano-4-(5-ethyl-3-methyl-1-(6-(2-methyl-1-oxoisoindolin-5-yl)-2-(methylsulfinyl)pyrimidin-4-yl)-1H-pyrazol-4-yl)-7-fluorothieno[3,2-c]pyridin-2-yl)carbamate

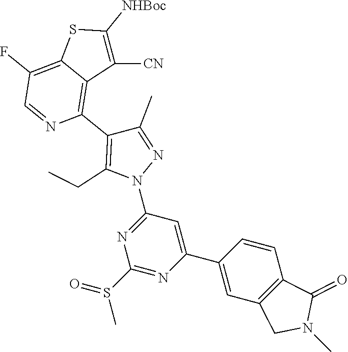 [SMILES: CCc1c(-c2ncc(F)c3sc(NC(=O)OC(C)(C)C)c(C#N)c23)c(C)nn1-c1cc(-c2ccc3c(c2)CN(C)C3=O)nc(S(C)=O)n1]

To a solution of tert-butyl (3-cyano-4-(5-ethyl-3-methyl-1-(6-(2-methyl-1-oxoisoindolin-5-yl)-2-(methylthio)pyrimidin-4-yl)-1H-pyrazol-4-yl)-7-fluorothieno[3,2-c]pyridin-2-yl)carbamate (210 mg, 0.313 mmol) in DCM (6 mL) at 0° C. was added mCPBA (81 mg, 0.470 mmol) The mixture was stirred at 0° C. for 30 min. then purified directly by FCC (0-20% MeOH in DCM) to afford the title compound (180 mg, 84%). LC-MS calc. for C₃₃H₃₂FN₈O₄S₂ (M+H)⁺: m/z=687.2; found 687.2.

### Step 3. 2-Amino-4-(5-ethyl-3-methyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile

LHMDS (1.0 M in THF, 582 μL, 0.582 mmol) was added to a solution of (S)-1-((S)-1-methylpyrrolidin-2-yl)ethan-1-ol (78 μl, 0.582 mmol) in anhydrous THF (3 mL). The mixture was stirred at 0° C. for 30 min. The resulting solution was added dropwise to a solution of tert-butyl (3-cyano-4-(5-ethyl-3-methyl-1-(6-(2-methyl-1-oxoisoindolin-5-yl)-2-(methylsulfinyl) pyrimidin-4-yl)-1H-pyrazol-4-yl)-7-fluorothieno[3,2-c]pyridin-2-yl)carbamate (200 mg, 0.291 mmol) in THF (3 mL) at 0° C. The mixture was stirred at 0° C. for 30 min. The reaction was quenched with sat. aq. NH₄Cl and extracted with EtOAc. The organic layer was dried over magnesium sulfate and concentrated. The crude product was purified by FCC (0-20% MeOH in DCM). The resulting Boc-protected intermediate was dissolved in 1:1 TFA/DCM (5 mL) and stirred at r.t. for 30 min. The mixture was then concentrated and purified by prep. LC-MS (XBRIDGE® C₁₈ column, eluting with a gradient of MeCN/water containing 0.1% TFA, at a flow rate of 60 mL/min.) to afford the product as a TFA salt. LC-MS calc. for C₃₄H₃₅FN₉O₂S (M+H)⁺: m/z=652.2; found 652.2. ¹H NMR (500 MHz, DMSO) δ 9.97 (t, J=5.6 Hz, 1H), 8.47 (s, 1H), 8.41 (s, 1H), 8.36 (d, J=7.3 Hz, 3H), 8.24 (s, 1H), 7.87 (d, J=7.9 Hz, 1H), 5.32 (dq, J=12.1, 6.1 Hz, 1H), 4.60 (s, 2H), 3.88-3.77 (m, 1H), 3.57 (dq, J=12.2, 6.2 Hz, 1H), 3.27 (dq, J=14.4, 7.2 Hz, 1H), 3.18 (dt, J=11.7, 6.1 Hz, 4H), 3.06 (d, J=4.7 Hz, 3H), 2.83 (dq, J=14.4, 7.3 Hz, 1H), 2.31 (dq, J=13.6, 7.1 Hz, 1H), 2.11 (s, 4H), 1.95 (dt, J=13.4, 6.9 Hz, 1H), 1.87 (tt, J=14.6, 6.3 Hz, 1H), 1.52 (d, J=6.0 Hz, 3H), 1.14 (t, J=7.3 Hz, 3H).

Diastereomers of Example 92 are understood to be:
2-amino-4-(5-ethyl-3-methyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Rₐ)-7-fluorothieno[3,2-c]pyridine-3-carbonitrile; and
2-amino-4-(5-ethyl-3-methyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Sₐ)-7-fluorothieno[3,2-c]pyridine-3-carbonitrile.

### Example 93. 2-Amino-4-(5-ethyl-3-methyl-1-(6-(2-methyl-1-oxoisoindolin-5-yl)-2-((3aS,6aS)-1-methylhexahydropyrrolo[3,4-b]pyrrol-5(1H)-yl)pyrimidin-4-yl)-1H-pyrazol-4-yl)-7-fluorothieno[3,2-c]pyridine-3-carbonitrile

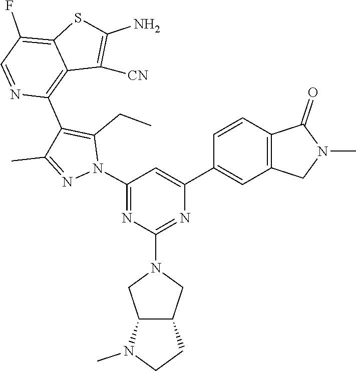 [SMILES: CCc1c(-c2ncc(F)c3sc(N)c(C#N)c23)c(C)nn1-c1cc(-c2ccc3c(c2)CN(C)C3=O)nc(N2C[C@@H]3CCN(C)[C@@H]3C2)n1]

A mixture of tert-butyl (3-cyano-4-(5-ethyl-3-methyl-1-(6-(2-methyl-1-oxoisoindolin-5-yl)-2-(methylsulfinyl)pyrimidin-4-yl)-1H-pyrazol-4-yl)-7-fluorothieno[3,2-c]pyridin-2-yl)carbamate (Example 92, Step 2, 200 mg, 0.291 mmol), (3aS,6aS)-1-methyloctahydropyrrolo[3,4-b]pyrrole (110 mg, 0.874 mmol) and DIPEA (153 μL, 0.874 mmol) in MeCN (3 mL) was heated to 100° C. for 10 min. The reaction was quenched by sat. aq. NH₄Cl and extracted with EtOAc. The organic layer was dried over Na₂SO₄ and concentrated. The crude product was purified by FCC (0-20% MeOH in DCM) to provide the desired intermediate. This was dissolved in 1:1 TFA/DCM solution (5 mL) and stirred at r.t. for 30 min. for full Boc deprotection. The mixture was then concentrated and purified by prep. LC-MS (XBRIDGE® Ca column, eluting with a gradient of MeCN/water containing 0.1% TFA, at a flow rate of 60 mL/min.) to afford the product as a TFA salt. Two isomers were obtained.

Peak 1, diastereomer 1. LC-MS calc. for C₃₄H₃₄FN₁₀OS (M+H)⁺: m/z=649.2; found 649.2. ¹H NMR (600 MHz, DMSO) δ 10.04 (s, 1H), 8.41 (d, J=5.0 Hz, 2H), 8.35 (d, J=6.2 Hz, 3H), 7.83 (d, J=7.4 Hz, 2H), 4.58 (s, 2H), 4.18 (q, J=6.9 Hz, 1H), 3.88 (dd, J=13.8, 6.3 Hz, 2H), 3.69 (d, J=11.3 Hz, 2H), 3.34 (qd, J=8.7, 4.3 Hz, 1H), 3.23 (q, J=9.1 Hz, 2H), 3.13 (s, 3H), 2.96 (m, 5H), 2.46 (dtd, J=13.0, 8.4, 4.0 Hz, 1H), 2.08 (s, 3H), 1.88 (s, 1H), 1.15 (t, J=7.2 Hz, 3H).

Peak 2, diastereomer 2. LC-MS calc. for C₃₄H₃₄FN₁₀0S (M+H)⁺: m/z=649.2; found 649.2.

Diastereomers of Example 93 are understood to be:
2-amino-4-(5-ethyl-3-methyl-1-(6-(2-methyl-1-oxoisoindolin-5-yl)-2-((3aS,6aS)-1-methylhexahydropyrrolo[3,4-b]pyrrol-5(1H)-yl)pyrimidin-4-yl)-1H-pyrazol-4-yl)-(Rₐ)-7-fluorothieno[3,2-c]pyridine-3-carbonitrile; and
2-amino-4-(5-ethyl-3-methyl-1-(6-(2-methyl-1-oxoisoindolin-5-yl)-2-((3aS,6aS)-1-methylhexahydropyrrolo[3,4-b]pyrrol-5(1H)-yl)pyrimidin-4-yl)-1H-pyrazol-4-yl)-(Sₐ)-7-fluorothieno[3,2-c]pyridine-3-carbonitrile.

### Example 94. 5-(6-(4-(2-Amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-methylpicolinamide

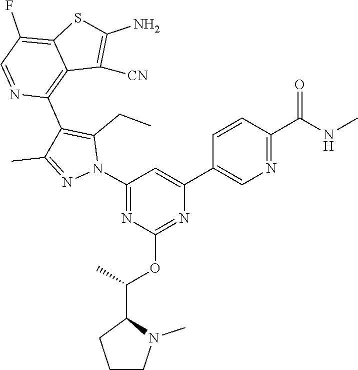 [SMILES: CCc1c(-c2ncc(F)c3sc(N)c(C#N)c23)c(C)nn1-c1cc(-c2ccc(C(=O)NC)nc2)nc(O[C@@H](C)[C@@H]2CCCN2C)n1]

The title compound was prepared according to the general procedures of Example 92, Steps 1-3, utilizing 2-(N-Methylamidocarboxy)-5-pyridine boronic acid pincol ester as the coupling partner in Step 1. LC-MS calc. for C₃₂H₃₄FN₁₀O₂S (M+H)⁺: m/z=641.2; found 641.2. ¹H NMR (600 MHz, DMSO) δ 10.09 (p, J=5.4 Hz, 1H), 9.42 (d, J=2.1 Hz, 1H), 8.87 (q, J=4.8 Hz, 1H), 8.79 (dd, J=8.1, 2.3 Hz, 1H), 8.41 (s, 1H), 8.37 (s, 2H), 8.29 (s, 1H), 8.22 (d, J=8.2 Hz, 1H), 5.33 (dq, J=10.0, 6.2 Hz, 1H), 3.87-3.78 (m, 1H), 3.57 (dq, J=12.2, 6.2 Hz, 1H), 3.27 (dq, J=14.4, 7.3 Hz, 1H), 3.17 (dq, J=12.5, 6.7 Hz, 1H), 3.05 (s, 3H), 2.88 (d, J=4.8 Hz, 3H), 2.82 (dq, J=14.5, 7.3 Hz, 1H), 2.34-2.26 (m, 1H), 2.14-2.05 (m, 4H), 1.93 (dp, J=13.1, 6.9 Hz, 1H), 1.90-1.81 (m, 1H), 1.52 (d, J=6.0 Hz, 3H), 1.13 (t, J=7.3 Hz, 3H).

Diastereomers of Example 94 are understood to be:
5-(6-((Rₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-methylpicolinamide; and
5-(6-((Sₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-methylpicolinamide.

### Example 95. 4-(6-(4-(2-Amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-2-fluoro-N-methylbenzamide

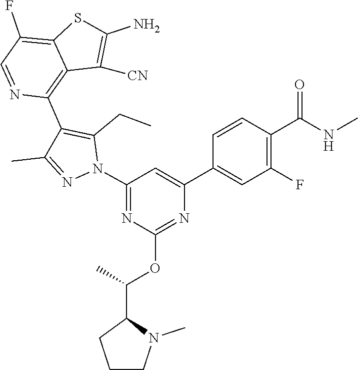 [SMILES: CCc1c(-c2ncc(F)c3sc(N)c(C#N)c23)c(C)nn1-c1cc(-c2ccc(C(=O)NC)c(F)c2)nc(O[C@@H](C)[C@@H]2CCCN2C)n1]

The title compound was prepared according to the general procedures of Example 92, Steps 1-3, utilizing (3-fluoro-4-(methylcarbamoyl)phenyl)boronic acid as the coupling partner in Step 1. After purification, two peaks were isolated.

Peak 1, diastereomer 1. LC-MS calc. for C₃₃H₃₄F₂N₉O₂S (M+H)⁺: m/z=658.2; found 658.2.

Peak 2, diastereomer 2. LC-MS calc. for C₃₃H₃₄F₂N₉O₂S (M+H)⁺: m/z=658.2; found 658.2.

Diastereomers of Example 95 are understood to be:
4-(6-((Rₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-2-fluoro-N-methylbenzamide; and
4-(6-((Sₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-2-fluoro-N-methylbenzamide.

### Example 96. 4-(6-(4-(2-Amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-cyclopropyl-2-fluorobenzamide

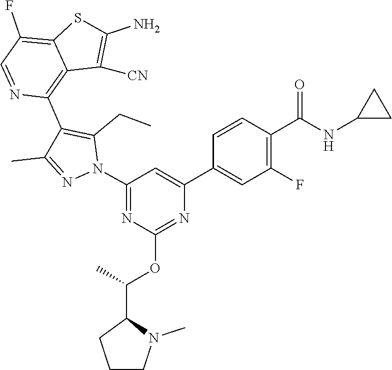 [SMILES: CCc1c(-c2ncc(F)c3sc(N)c(C#N)c23)c(C)nn1-c1cc(-c2ccc(C(=O)NC3CC3)c(F)c2)nc(O[C@@H](C)[C@@H]2CCCN2C)n1]

The title compound was prepared according to the general procedures of Example 92, Steps 1-3, utilizing (4-(cyclopropylcarbamoyl)-3-fluorophenyl)boronic acid as the coupling partner in Step 1. After purification, two peaks were isolated.

Peak 1, diastereomer 1. LC-MS calc. for C₃₅H₃₆F₂N₉O₂S (M+H)⁺: m/z=684.2; found 684.2.

Peak 2, diastereomer 2. LC-MS calc. for C₃₅H₃₆F₂N₉O₂S (M+H)⁺: m/z=684.2; found 684.2.

Diastereomers of Example 96 are understood to be:
4-(6-((Rₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-cyclopropyl-2-fluorobenzamide; and
4-(6-((Sₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-cyclopropyl-2-fluorobenzamide.

### Example 97. 5-(6-(4-(2-Amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-3-fluoro-N-methylpicolinamide

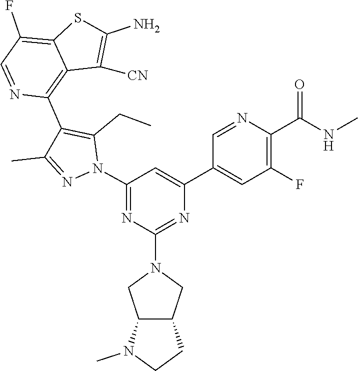 [SMILES: CCc1c(-c2ncc(F)c3sc(N)c(C#N)c23)c(C)nn1-c1cc(-c2cnc(C(=O)NC)c(F)c2)nc(N2C[C@@H]3CCN(C)[C@@H]3C2)n1]

### Step 1. 5-Bromo-3-fluoro-N-methylpicolinamide

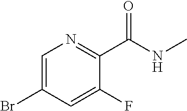

A solution of 5-bromo-3-fluoropicolinic acid (2 g, 9.09 mmol), HATU (5.19 g, 13.64 mmol) and DIPEA (3.18 mL, 18.18 mmol) in DMF (60 mL) was stirred at r.t. for 15 min., then methanamine (2.0 M in THF, 6.82 mL, 13.64 mmol) was added. The mixture was stirred at r.t. for 1 h. The reaction was diluted with water and extracted with EtOAc. The organic layer was washed with brine, dried over Na₂SO₄ and concentrated. The crude product was purified by FCC (0-100% EtOAc in hexanes) to provide the title compound. LC-MS calc. for C₇H₇BrFN₂O (M+H)⁺: m/z=233.0, 235.0; found 232.9, 234.9.

### Step 2. 3-Fluoro-N-methyl-5-(4,4,5,5-tetramethyl-1,3,2-dioxaborolan-2-yl)picolinamide

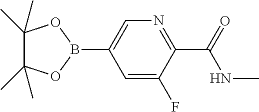

A mixture of 5-bromo-3-fluoro-N-methylpicolinamide (2.0 g, 8.58 mmol), bis(pinacolato)diboron (3.3 g, 12.87 mmol), potassium acetate (2.6 g, 25.7 mmol), (dppf)PdCl₂ (628 mg, 0.858 mmol) in anhydrous 1,4-dioxane (57 mL) was deoxygenated with N₂, then stirred at 100° C. for 1 h. The mixture was cooled to ambient temperature and poured into ice. The mixture was extracted with EtOAc (3×50 mL). The organic layer was washed with brine, dried over Na₂SO₄ and concentrated. The crude product was purified by FCC (0-20% MeOH in DCM) to provide the title compound. LC-MS calc. for C₁₃H₁₉BFN₂O₃ (M+H)⁺: m/z=281.1; found 281.1.

### Step 3. tert-Butyl (3-cyano-4-(5-ethyl-1-(6-(5-fluoro-6-(methylcarbamoyl)pyridin-3-yl)-2-(methylthio)pyrimidin-4-yl)-3-methyl-1H-pyrazol-4-yl)-7-fluorothieno[3,2-c]pyridin-2-yl)carbamate

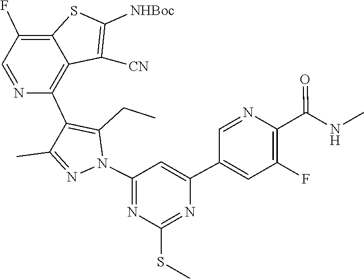 [SMILES: CCc1c(-c2ncc(F)c3sc(NC(=O)OC(C)(C)C)c(C#N)c23)c(C)nn1-c1cc(-c2cnc(C(=O)NC)c(F)c2)nc(SC)n1]

To a 40 mL vial were added Intermediate 10 (375 mg, 0.670 mmol), 3-fluoro-N-methyl-5-(4,4,5,5-tetramethyl-1,3,2-dioxaborolan-2-yl)picolinamide (375 mg, 1.339 mmol), potassium phosphate tribasic (284 mg, 1.339 mmol), and XPhos Pd G2 (105 mg, 0.134 mmol). The vial was then evacuated and an inert atmosphere was established. Dioxane (5.5 mL) and water (1.1 mL) were then added and the mixture was heated to 100° C. for 1 h. The mixture was cooled to r.t., quenched with water and extracted with EtOAc. The organic layer was dried over MgSO₄, concentrated and used directly in the next step without further purification. LC-MS calc. for C₃₁H₃₀F₂N₉O₃S₂ (M+H)⁺: m/z=678.2; found 678.2.

### Step 4. tert-Butyl (3-cyano-4-(5-ethyl-1-(6-(5-fluoro-6-(methylcarbamoyl)pyridin-3-yl)-2-(methylsulfinyl)pyrimidin-4-yl)-3-methyl-1H-pyrazol-4-yl)-7-fluorothieno[3,2-c]pyridin-2-yl)carbamate

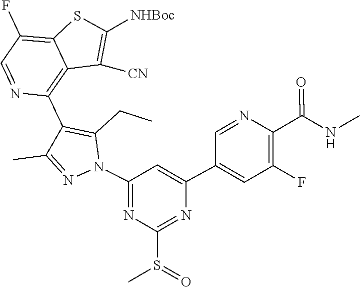 [SMILES: CCc1c(-c2ncc(F)c3sc(NC(=O)OC(C)(C)C)c(C#N)c23)c(C)nn1-c1cc(-c2cnc(C(=O)NC)c(F)c2)nc(S(C)=O)n1]

To a solution of tert-butyl (3-cyano-4-(5-ethyl-1-(6-(5-fluoro-6-(methylcarbamoyl)pyridin-3-yl)-2-(methylthio)pyrimidin-4-yl)-3-methyl-1H-pyrazol-4-yl)-7-fluorothieno[3,2-c]pyridin-2-yl)carbamate (450 mg, 0.664 mmol) in DCM (6.6 mL) at 0° C. was added mCPBA (344 mg, 1.992 mmol). The mixture was stirred at 0° C. for 30 min. The mixture was directly loaded onto a silica gel column and purified by FCC (0-15% MeOH in DCM) to provide the title compound (320 mg, 70% yield). LC-MS calc. for C₃₁H₃₀F₂N₉O₄S₂ (M+H)⁺: m/z=694.2; found 694.2.

### Step 5. 5-(6-(4-(2-Amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-3-fluoro-N-methylpicolinamide

A mixture of tert-butyl (3-cyano-4-(5-ethyl-1-(6-(5-fluoro-6-(methylcarbamoyl)pyridin-3-yl)-2-(methylsulfinyl)pyrimidin-4-yl)-3-methyl-1H-pyrazol-4-yl)-7-fluorothieno[3,2-c]pyridin-2-yl)carbamate (300 mg, 0.432 mmol), (3aS,6aS)-1-methyloctahydropyrrolo[3,4-b]pyrrole (82 mg, 0.649 mmol) and DIPEA (227 μL, 1.297 mmol) in MeCN was heated to 65° C. for 15 min. The mixture was diluted with water and EtOAc and the layes were separated. The organic layer was washed with brine, dried over magnesium sulfate and concentrated. The crude product was purified by FCC (0-15% MeOH in DCM) to provide the desired intermediate, which was dissolved in 1:1 TFA/DCM (10 mL) and stirred at r.t. for 1 h to provide full Boc deprotection. The mixture was concentrated and purified by prep. LC-MS (XBRIDGE® C₁8 column, eluting with a gradient of MeCN/water containing 0.1% TFA, at a flow rate of 60 mL/min.) to afford the product as a TFA salt. Two isomers were obtained.

Peak 1, diastereomer 1. LC-MS calc. for C₃₂H₃₂F₂N₁₁OS (M+H)⁺: m/z=656.2; found 656.2.

Peak 2, diastereomer 2. LC-MS calc. for C₃₂H₃₂F₂N₁₁OS (M+H)⁺: m/z=656.2; found 656.2. ¹H NMR (500 MHz, DMSO) δ 9.22 (s, 1H), 8.54 (dd, J=11.6, 1.7 Hz, 1H), 8.50 (m, 1H), 8.37 (s, 1H), 8.14 (s, 2H), 7.89 (s, 1H), 4.22 (s, 3H), 3.92 (dd, J=13.6, 7.2 Hz, 2H), 3.73-3.62 (m, 2H), 3.36 (s, 1H), 3.25 (dt, J=14.4, 7.2 Hz, 2H), 3.02-2.93 (m, 3H), 2.87 (d, J=4.7 Hz, 3H), 2.53-2.41 (m, 3H), 2.10 (s, 3H), 1.97-1.88 (m, 1H), 1.15 (t, J=7.3 Hz, 3H).

Diastereomers of Example 97 are understood to be:
5-(6-((Rₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-3-fluoro-N-methylpicolinamide; and
5-(6-((Sₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-3-fluoro-N-methylpicolinamide.

### Example 98. 4-(6-(4-(2-Amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-2-fluorobenzamide

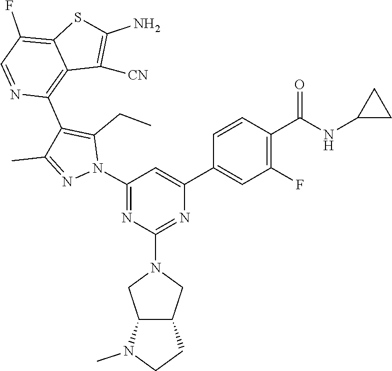 [SMILES: CCc1c(-c2ncc(F)c3sc(N)c(C#N)c23)c(C)nn1-c1cc(-c2ccc(C(=O)NC3CC3)c(F)c2)nc(N2C[C@@H]3CCN(C)[C@@H]3C2)n1]

The title compound was prepared according to the general procedures of Example 97, Steps 3-5, utilizing (4-(cyclopropylcarbamoyl)-3-fluorophenyl)boronic acid as the coupling partner in Step 3. After purification, two peaks were isolated.

Peak 1, diastereomer 1. LC-MS calc. for C₃₅H₃₅F₂N₁₀OS (M+H)⁺: m/z=681.2; found 681.2.

Peak 2, diastereomer 2. LC-MS calc. for C₃₅H₃₅F₂N₁₀OS (M+H)⁺: m/z=681.2; found 681.2.

Diastereomers of Example 98 are understood to be:
4-(6-((Rₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-2-fluorobenzamide; and
4-(6-((Sₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-2-fluorobenzamide.

### Example 99. 5-(6-(4-(2-Amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-3-fluoropicolinamide

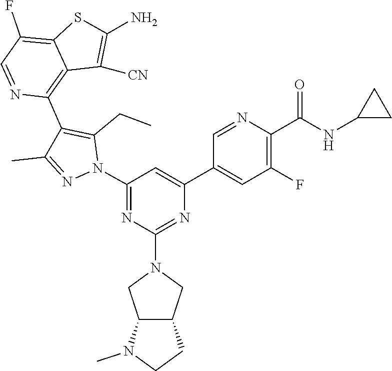 [SMILES: CCc1c(-c2ncc(F)c3sc(N)c(C#N)c23)c(C)nn1-c1cc(-c2cnc(C(=O)NC3CC3)c(F)c2)nc(N2C[C@@H]3CCN(C)[C@@H]3C2)n1]

The title compound was prepared according to the general procedures of Example 97, Steps 1-5, utilizing cyclopropyl amine as the coupling partner in Step 1. After purification, two peaks were isolated.

Peak 1, diastereomer 1. LC-MS calc. for C₃₄H₃₄F₂N₁₁OS (M+H)⁺: m/z=682.2; found 682.2.

Peak 2, diastereomer 2. LC-MS calc. for C₃₄H₃₄F₂N₁₁OS (M+H)⁺: m/z=682.2; found 682.2.

Diastereomers of Example 99 are understood to be: 5-(6-((Rₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-3-fluoropicolinamide; and

5-(6-((Sₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-3-fluoropicolinamide.

### Example 100. 2-Amino-4-(5-ethyl-1-(6-(2-methyl-1-oxoisoindolin-5-yl)-2-((S)-1-((S)-1-methylpyrrolidin-2-yl)ethoxy)pyrimidin-4-yl)-3-(trifluoromethyl)-1H-pyrazol-4-yl)-7-fluorothieno[3,2-c]pyridine-3-carbonitrile

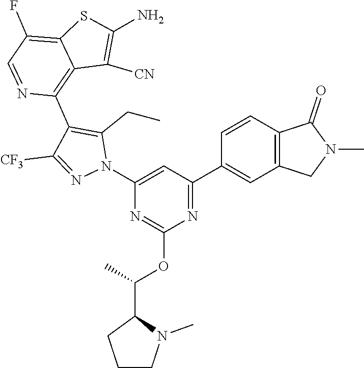 [SMILES: CCc1c(-c2ncc(F)c3sc(N)c(C#N)c23)c(C(F)(F)F)nn1-c1cc(-c2ccc3c(c2)CN(C)C3=O)nc(O[C@@H](C)[C@@H]2CCCN2C)n1]

### Step 1. tert-butyl 4-bromo-5-ethyl-3-(trifluoromethyl)-1H-pyrazole-1-carboxylate

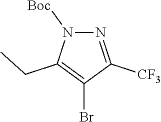 [SMILES: CCc1c(Br)c(C(F)(F)F)nn1C(=O)OC(C)(C)C]

To a solution of 4-bromo-3-ethyl-5-(trifluoromethyl)-1H-pyrazole (0.75 g, 3.09 mmol) in THF (20.57 mL) in a round-bottom flask were added (Boc)₂0 (1.075 mL, 4.63 mmol), NEt₃ (0.860 mL, 6.17 mmol) and DMAP (0.038 g, 0.309 mmol). The resulting mixture was stirred at r.t. for 30 min. Upon completion, the reaction mixture was then concentrated under reduced pressure. The crude product were purified by FCC (0-20% EtOAc in Hexanes) to provide the title compound (810 mg, 76%). LC-MS calc. for C₆H₇BrF₃N₂(M-Boc)⁺: m/z=243.0, 245.0; found 243.0, 245.0.

### Step 2. tert-butyl3-ethyl-4-(4,4,5,5-tetramethyl-1,3,2-dioxaborolan-2-yl)-5-(trifluoromethyl)-1H-pyrazole-1-carboxylate

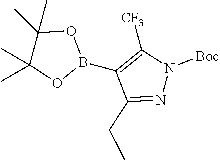 [SMILES: CCc1nn(C(=O)OC(C)(C)C)c(C(F)(F)F)c1B1OC(C)(C)C(C)(C)O1]

Under N₂, the reaction mixture of tert-butyl 4-bromo-3-ethyl-5-(trifluoromethyl)-1H-pyrazole-1-carboxylate (1000 mg, 2.91 mmol) and 2-isopropoxy-4,4,5,5-tetramethyl-1,3,2-dioxaborolane (1784 μl, 8.74 mmol) in THF (9.7 mL) at −78° C. was added i-butyllithium (5464 μl, 8.74 mmol). The reaction mixture was stirred at −78° C. for 2 h and slowly warmed to r.t. The reaction mixture was then quenched with NH₄Cl solution and extracted with EtOAc. The organic layers were dried over MgSO₄, concentrated and purified through FCC (0-15% EtOAc in Hexanes) to provide the title compound (850 mg, 75%). LC-MS calc. for C₁₂H₁₉BF₃N₂O₂ (M-Boc)⁺: m/z=291.1; found 291.1.

### Step 3. tert-butyl (3-cyano-4-(5-ethyl-3-(trifluoromethyl)-1H-pyrazol-4-yl)-7-fluorothieno[3,2-c]pyridin-2-yl)carbamate

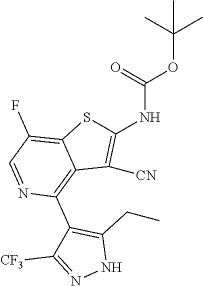 [SMILES: CCc1[nH]nc(C(F)(F)F)c1-c1ncc(F)c2sc(NC(=O)OC(C)(C)C)c(C#N)c12]

Under N₂, to a reaction mixture of tert-butyl (4-chloro-3-cyano-7-fluorothieno[3,2-c]pyridin-2-yl)carbamate (550 mg, 1.678 mmol), tert-butyl 5-ethyl-4-(4,4,5,5-tetramethyl-1,3,2-dioxaborolan-2-yl)-3-(trifluoromethyl)-1H-pyrazole-1-carboxylate (851 mg, 2.181 mmol) in dioxane (13.4 mL)/water (3.4 mL) were added RuPhos Pd G4 (285 mg, 0.336 mmol), RuPhos (157 mg, 0.336 mmol) and tripotassium phosphate (1069 mg, 5.03 mmol). The reaction mixture was stirred at 100° C. for 1 h. The reaction mixture was cooled to r.t. and poured into water. The aqueous layer was extracted 3 times with EtOAc. The organic layers were dried over MgSO₄, concentrated, and purified by FCC (0-100% EtOAc in Hexanes) to provide the title compound as light yellow solid (410 mg, 44%). LC-MS calc. for C₁₉H₁₈F₄N₅O₂S (M+H)⁺: m/z=456.1; found 456.1.

### Step 4. tert-butyl (3-cyano-4-(5-ethyl-1-(6-(2-methyl-1-oxoisoindolin-5-yl)-2-(methylthio)pyrimidin-4-yl)-3-(trifluoromethyl)-1H-pyrazol-4-yl)-7-fluorothieno[3,2-c]pyridin-2-yl)carbamate

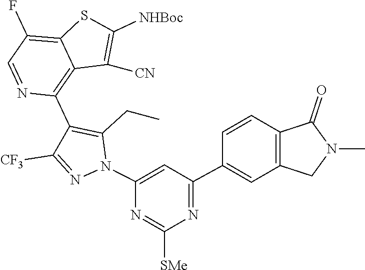 [SMILES: CCc1c(-c2ncc(F)c3sc(NC(=O)OC(C)(C)C)c(C#N)c23)c(C(F)(F)F)nn1-c1cc(-c2ccc3c(c2)CN(C)C3=O)nc(SC)n1]

To a solution of tert-butyl (3-cyano-4-(5-ethyl-3-(trifluoromethyl)-1H-pyrazol-4-yl)-7-fluorothieno[3,2-c]pyridin-2-yl)carbamate (200 mg, 0.439 mmol) in MeCN (4.4 mL) was added 5-(6-chloro-2-(methylthio)pyrimidin-4-yl)-2-methylisoindolin-1-one (168 mg, 0.549 mmol) and Cs₂CO₃ (286 mg, 0.878 mmol). The reaction mixture stirred at 90° C. for 2 h. Upon completion, the reaction mixture was cooled to r.t. and filtered over a pad of diatomaceous earth. The filtrate was concentrated over reduced pressure and subject to FCC (0-100% EtOAc in Hexanes) to provide the title compound as white solid (210 mg, 66%). LC-MS calc. for C₃₃H₂₉F₄N₈O₃S₂ (M+H)⁺: m/z=725.2; found 725.2.

### Step 5. tert-butyl (3-cyano-4-(5-ethyl-1-(6-(2-methyl-1-oxoisoindolin-5-yl)-2-(methylsulfinyl)pyrimidin-4-yl)-3-(trifluoromethyl)-1H-pyrazol-4-yl)-7-fluorothieno[3,2-c]pyridin-2-yl)carbamate

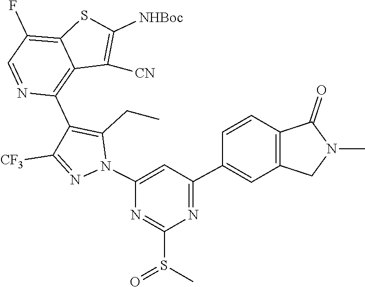 [SMILES: CCc1c(-c2ncc(F)c3sc(NC(=O)OC(C)(C)C)c(C#N)c23)c(C(F)(F)F)nn1-c1cc(-c2ccc3c(c2)CN(C)C3=O)nc(S(C)=O)n1]

To a solution of tert-butyl (3-cyano-4-(5-ethyl-1-(6-(2-methyl-1-oxoisoindolin-5-yl)-2-(methylthio)pyrimidin-4-yl)-3-(trifluoromethyl)-1H-pyrazol-4-yl)-7-fluorothieno[3,2-c]pyridin-2-yl)carbamate (200 mg, 0.276 mmol) in DCM (5.5 mL) at 0° C. was added m-CPBA (71.4 mg, 0.414 mmol). The reaction mixture was stirred at 0° C. for 10 min. Upon completion, the reaction mixture was quenched by aqueous NaHCO₃ and sodium thiosulfate solution. The aqueous layer was then extracted with DCM and the resulting organic layers were dried over MgSO₄ and concentrated directly to give pure product which was used directly for the next step. (200 mg, 96%). LC-MS calc. for C₃₃H₂₉F₄N₈O₄S₂(M+H)⁺: m/z=741.2; found 741.1.

### Step 6. 2-amino-4-(5-ethyl-1-(6-(2-methyl-1-oxoisoindolin-5-yl)-2-((S)-1-((S)-1-methylpyrrolidin-2-yl)ethoxy)pyrimidin-4-yl)-3-(trifluoromethyl)-1H-pyrazol-4-yl)-7-fluorothieno[3,2-c]pyridine-3-carbonitrile

LiHMDS (202 μL, 0.202 mmol) was added to a solution of tert-butyl (3-cyano-4-(5-ethyl-1-(6-(2-methyl-1-oxoisoindolin-5-yl)-2-(methylsulfinyl)pyrimidin-4-yl)-3-(trifluoromethyl)-1H-pyrazol-4-yl)-7-fluorothieno[3,2-c]pyridin-2-yl)carbamate (30 mg, 0.040 mmol) and (S)-1-((S)-1-methylpyrrolidin-2-yl)ethan-1-ol (27.3 μl, 0.202 mmol) in THF (405 μL) at 0° C. The reaction mixture was moved to r.t. and stirred for 30 min. Upon completion, the reaction was then dissolved in 1:1 TFA/DCM solution (5 mL) and stirred at r.t. for 30 min. The reaction mixture was then concentrated and purified by preparative LC-MS (XBRIDGE® C₁₈ column, eluting with a gradient of MeCN/water containing 0.1% TFA, at flow rate of 60 mL/min) to afford the product as a TFA salt. The two isomers were inseparable. Only one peak was obtained. LC-MS calc. for C₃₄H₃₂F₄N₉O₂S (M+H)⁺: m/z=706.2; found 706.2.

Diastereomers of Example 100 are understood to be: 2-amino-4-(5-ethyl-1-(6-(2-methyl-1-oxoisoindolin-5-yl)-2-((S)-1-((S)-1-methylpyrrolidin-2-yl)ethoxy)pyrimidin-4-yl)-3-(trifluoromethyl)-1H-pyrazol-4-yl)-(Rₐ)-7-fluorothieno[3,2-c]pyridine-3-carbonitrile; and

2-amino-4-(5-ethyl-1-(6-(2-methyl-1-oxoisoindolin-5-yl)-2-((S)-1-((S)-1-methylpyrrolidin-2-yl)ethoxy)pyrimidin-4-yl)-3-(trifluoromethyl)-1H-pyrazol-4-yl)-(Sₐ)-7-fluorothieno[3,2-c]pyridine-3-carbonitrile.

### Example 101. 2-Amino-4-(3,5-diethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile

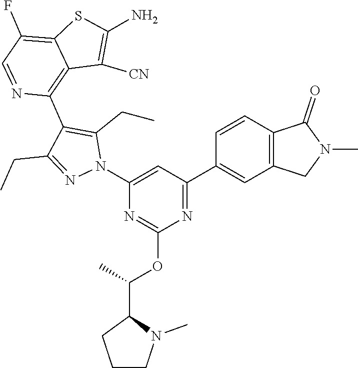 [SMILES: CCc1nn(-c2cc(-c3ccc4c(c3)CN(C)C4=O)nc(O[C@@H](C)[C@@H]3CCCN3C)n2)c(CC)c1-c1ncc(F)c2sc(N)c(C#N)c12]

The title compound was prepared according to the procedure described for Example 100, replacing 4-bromo-3-ethyl-5-(trifluoromethyl)-1H-pyrazole with 4-bromo-3,5-diethyl-1H-pyrazole in Step 1. Two peaks were isolated.

Peak 1, diastereomer 1. LC-MS calc. for C₃₅H₃₇FN₉O₂S (M+H)⁺: m/z=666.3; found 666.3.

Peak 2, diastereomer 2. LC-MS calc. for C₃₅H₃₇FN₉O₂S (M+H)⁺: m/z=666.3; found 666.3.

Diastereomers of Example 101 are understood to be: 2-amino-4-(3,5-diethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Rₐ)-7-fluorothieno[3,2-c]pyridine-3-carbonitrile; and

2-amino-4-(3,5-diethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Sₐ)-7-fluorothieno[3,2-c]pyridine-3-carbonitrile.

### Example 102. (S)-2-Amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-7-fluorothieno[3,2-c]pyridine-3-carbonitrile

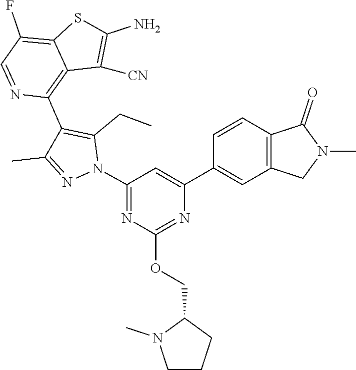 [SMILES: CCc1c(-c2ncc(F)c3sc(N)c(C#N)c23)c(C)nn1-c1cc(-c2ccc3c(c2)CN(C)C3=O)nc(OC[C@@H]2CCCN2C)n1]

The title compound was prepared according to the procedures described for Example 92, by replacing (S)-1-((S)-1-methylpyrrolidin-2-yl)ethan-1-ol with (S)-(1-methylpyrrolidin-2-yl)methanol in Step 3. One peak was obtained. LC-MS calc. for C₃₃H₃₃FN₉O₂S (M+H)⁺: m/z=638.2; found 638.2.

Diastereomers of Example 102 are understood to be: (S)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-(Rₐ)-7-fluorothieno[3,2-c]pyridine-3-carbonitrile; and

(S)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-(Sₐ)-7-fluorothieno[3,2-c]pyridine-3-carbonitrile.

### Examples 103-105

The following compounds listed in Table 13 were prepared in an analogous fashion to Example 93 using the appropriate amines.

TABLE 13
Examples 103-105

| Ex | | | ESI |
|---|---|---|---|
| # | Structure | IUPAC name | [M + H]⁺ |
| 103 | 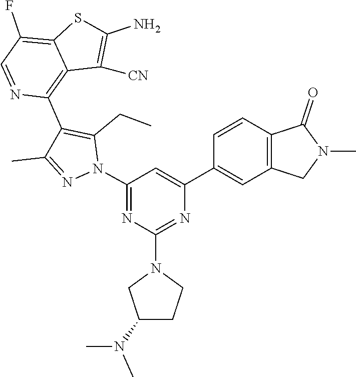 [SMILES: CCc1c(-c2ncc(F)c3sc(N)c(C#N)c23)c(C)nn1-c1cc(-c2ccc3c(c2)CN(C)C3=O)nc(N2CC[C@H](N(C)C)C2)n1] | (S)-2-Amino-4-(1-(2-(3- (dimethylamino)pyrrolidin-1-yl)-6-(2- methyl-1-oxoisoindolin-5-yl)pyrimidin-4- yl)-5-ethyl-3-methyl-1H-pyrazol-4-yl)-7- fluorothieno[3,2-c]pyridine-3-carbonitrile | 637.3 |
| 104 | 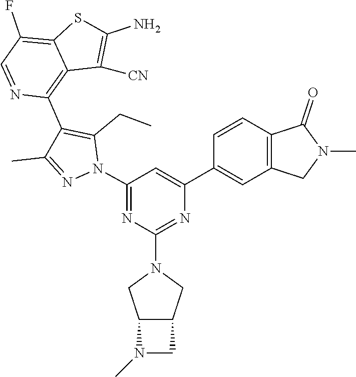 [SMILES: CCc1c(-c2ncc(F)c3sc(N)c(C#N)c23)c(C)nn1-c1cc(-c2ccc3c(c2)CN(C)C3=O)nc(N2C[C@@H]3CN(C)[C@@H]3C2)n1] | 2-Amino-4-(5-ethyl-3-methyl-1-(6-(2- methyl-1-oxoisoindolin-5-yl)-2-((1S,5S)-6- methyl-3,6-diazabicyclo[3.2.0]heptan-3- yl)pyrimidin-4-yl)-1H-pyrazol-4-yl)-7- fluorothieno[3,2-c]pyridine-3-carbonitrile | 635.2 |
| 105 | 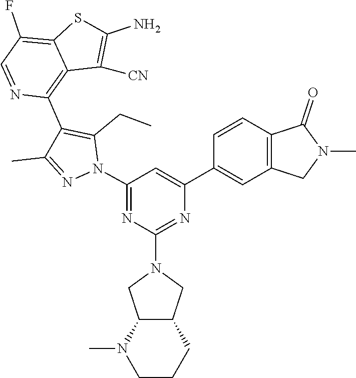 [SMILES: CCc1c(-c2ncc(F)c3sc(N)c(C#N)c23)c(C)nn1-c1cc(-c2ccc3c(c2)CN(C)C3=O)nc(N2C[C@@H]3CCCN(C)[C@@H]3C2)n1] | 2-Amino-4-(5-ethyl-3-methyl-1-(6-(2- methyl-1-oxoisoindolin-5-yl)-2-((4aS,7aS)- 1-methyloctahydro-6H-pyrrolo[3,4- b]pyridin-6-yl)pyrimidin-4-yl)-1H-pyrazol- 4-yl)-7-fluorothieno[3,2-c]pyridine-3- carbonitrile | 663.3 |

Diastereomers of Example 103 are understood to be: (S)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile; and

(S)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile.

Diastereomers of Example 104 are understood to be: 2-amino-4-(5-ethyl-3-methyl-1-(6-(2-methyl-1-oxoisoindolin-5-yl)-2-((1 S,5S)-6-methyl-3,6-diazabicyclo[3.2.0]heptan-3-yl)pyrimidin-4-yl)-1H-pyrazol-4-yl)-(Rₐ)-7-fluorothieno[3,2-c]pyridine-3-carbonitrile; and

2-amino-4-(5-ethyl-3-methyl-1-(6-(2-methyl-1-oxoisoindolin-5-yl)-2-((1 S,5S)-6-methyl-3,6-diazabicyclo[3.2.0]heptan-3-yl)pyrimidin-4-yl)-1H-pyrazol-4-yl)-(Sₐ)-7-fluorothieno[3,2-c]pyridine-3-carbonitrile.

Diastereomers of Example 105 are understood to be: 2-amino-4-(5-ethyl-3-methyl-1-(6-(2-methyl-1-oxoisoindolin-5-yl)-2-((4aS,7aS)-1-methyloctahydro-6H-pyrrolo[3,4-b]pyridin-6-yl)pyrimidin-4-yl)-1H-pyrazol-4-yl)-(Rₐ)-7-fluorothieno[3,2-c]pyridine-3-carbonitrile; and

2-amino-4-(5-ethyl-3-methyl-1-(6-(2-methyl-1-oxoisoindolin-5-yl)-2-((4aS,7aS)-1-methyloctahydro-6H-pyrrolo[3,4-b]pyridin-6-yl)pyrimidin-4-yl)-1H-pyrazol-4-yl)-(Sₐ)-7-fluorothieno[3,2-c]pyridine-3-carbonitrile.

### Example 106: 2-Amino-4-(1-(2-((3aS,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile

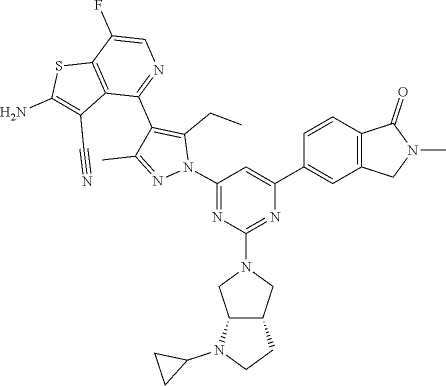 [SMILES: CCc1c(-c2ncc(F)c3sc(N)c(C#N)c23)c(C)nn1-c1cc(-c2ccc3c(c2)CN(C)C3=O)nc(N2C[C@@H]3CCN(C4CC4)[C@@H]3C2)n1]

### Step 1. (3aS,6aS)-1-cyclopropyloctahydropyrrolo[3,4-b]pyrrole

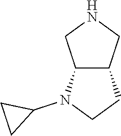 [SMILES: C1CC1N1CC[C@H]2CNC[C@H]21]

To a stirred solution of tert-butyl (3aS,6aS)-hexahydropyrrolo[3,4-b]pyrrole-5(1H)-carboxylate (100 mg, 0.471 mmol) and ((1-ethoxycyclopropyl)oxy)trimethylsilane (0.474 mL, 2.355 mmol) in MeOH (2 mL) were added molecular sieves (50 mg), followed by AcOH (0.404 mL, 7.07 mmol) and sodium cyanoborohydride (89 mg, 1.413 mmol). The mixture was heated to reflux for 4 h. Subsequently, the mixture was diluted with EtOAc and saturated aqueous NaHCO₃. The organic layer was washed twice with NaHCO₃, followed by a brine wash and was dried over Na₂SO₄. The organic layer was then filtered and concentrated. The concentrate was resuspended in 2 mL MeCN and 2 mL TFA and was stirred at rt for 2 h. The mixture was subsequently concentrated to dryness and was taken forward for subsequent reactions. LC-MS calc. for C₉H₁₇N₂(M+H)⁺: m/z=153.2; found 153.2.

### Step 2. 2-amino-4-(1-(2-((3aS,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile

A stirred mixture of tert-butyl (3-cyano-4-(5-ethyl-3-methyl-1-(6-(2-methyl-1-oxoisoindolin-5-yl)-2-(methylsulfinyl)pyrimidin-4-yl)-1H-pyrazol-4-yl)-7-fluorothieno[3,2-c]pyridin-2-yl)carbamate (Example 92, Step 2, 20 mg, 0.029 mmol), (3aS,6aS)-1-cyclopropyloctahydropyrrolo[3,4-b]pyrrole (4.43 mg, 0.029 mmol) and DIPEA (0.102 ml, 0.582 mmol) in DMF (0.5 ml) was heated at 100° C. for 1 h. The reaction mixture was then cooled to r.t., diluted with MeCN (3.5 mL) and TFA (1 mL) and stirred at r.t. for 2 h. The reaction mixture was then filtered and purified by preparative LC-MS (XBRIDGE® C₁₈ column, eluting with a gradient of MeCN/water containing 0.1% TFA, at flow rate of 60 mL/min.) to afford the product as a TFA salt. LC-MS calc. for C₃₆H₃₆FN₁₀OS (M+H)⁺: m/z=675.3; found 675.3.

Diastereomers of Example 106 are understood to be: 2-amino-4-(1-(2-((3aS,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile; and

2-amino-4-(1-(2-((3aS,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile.

### Example A. GDP-GTP Exchange Assay

The inhibitor potency of the exemplified compounds was determined in a fluorescence based guanine nucleotide exchange assay, which measures the exchange of bodipy-GDP (fluorescently labeled GDP) for GppNHp (Non-hydrolyzable GTP analog) to generate the active state of KRAS in the presence of SOS1 (guanine nucleotide exchange factor). Inhibitors were serially diluted in DMSO and a volume of 0.1 μL was transferred to the wells of a black low volume 384-well plate. 5 μL/well volume of bodipy-loaded KRAS G12V diluted to 2.5 nM in assay buffer (25 mM Hepes pH 7.5, 50 mM NaCl, 10 mM MgCl₂ and 0.01% Brij-35) was added to the plate and pre-incubated with inhibitor for 4 h at r.t. Appropriate controls (enzyme with no inhibitor or with a G12V inhibitor) were included on the plate. The exchange was initiated by the addition of a 5 μL/well volume containing 1 mM GppNHp and 300 nM SOS1 in assay buffer. The 10 μL/well reaction concentration of the bodipy-loaded KRAS G12V, GppNHp, and SOS1 were 2.5 nM, 500 μM, and 150 nM, respectively. The reaction plates were incubated at r.t. for 2 h, a time estimated for complete GDP-GTP exchange in the absence of inhibitor. For the WT, similar guanine nucleotide exchange assays were used with 2.5 nM as final concentration for the bodipy loaded KRAS proteins and 3 h incubation after adding GppNHp-SOS1 mixture. A cyclic peptide described to selectively bind G12V mutant (Sakamoto et al., BBRC 484.3 (2017), 605-611) or internal compounds with confirmed binding were used as positive controls in the assay plates. Fluorescence intensities were measured on a PheraStar plate reader instrument (BMG Labtech) with excitation at 485 nm and emission at 520 nm.

Either GraphPad prism or Genedata Screener SmartFit was used to analyze the data. The IC₅₀ values were derived by fitting the data to a four parameter logistic equation producing a sigmoidal dose-response curve with a variable Hill coefficient.

The KRAS_G12V and WT exchange assay IC₅₀ data are provided in Table A below. The symbol “†” indicates IC₅₀<100 nM, “††” indicates IC₅₀>100 nM but s 1 μM; and “†††” indicates IC₅₀ is >1 μM but ≤5 μM, “††††” indictes IC₅₀ is >5 μM but ≤10 μM. “NA” indicates IC₅₀ not available.

TABLE A

| Ex. No. | G12V_exchange | WT_exchange |
|---|---|---|
| 1 | † | † |
| 2 | † | †† |
| 3 | † | †† |
| 4 | †† | †† |
| 5 | † | † |
| 6 | †† | †† |
| 7 | † | † |
| 8 | † | † |
| 9 | † | † |
| 10 | †† | †† |
| 11 | †† | †† |
| 12 | † | † |
| 13 | † | † |
| 14 | † | † |
| 15 | † | † |
| 16 | † | †† |
| 17 | † | † |
| 18 | † | † |
| 19 | †† | †† |
| 20 | † | † |
| 21 | † | † |
| 22 | † | †† |
| 23 | † | † |
| 24 | † | † |
| 25 | † | † |
| 26 | † | †† |
| 27 | † | † |
| 28 | † | † |
| 29 | † | †† |
| 30 | † | † |
| 31 | † | † |
| 32 | † | † |
| 33 | † | † |
| 34 | † | †† |
| 35 | †† | †† |
| 36 | †† | † |
| 37 | † | † |
| 38 | † | † |
| 39 | † | † |
| 40 | † | † |
| 41 | † | † |
| 42 | † | † |
| 43 | † | † |
| 44 | † | † |
| 45 | † | † |
| 46 | † | † |
| 47 | † | † |
| 48 | † | † |
| 49 | †† | † |
| 50 | † | † |
| 51 | † | †† |
| 52 | †† | †††† |
| 53 | †† | †† |
| 54 | † | †† |
| 55 | ††† | †††† |
| 56 | ††† | †††† |
| 57 | † | † |
| 58 | † | † |
| 59 | † | † |
| 60 | † | † |
| 61 | † | † |
| 62 | † | † |
| 63 | † | †† |
| 64 | † | † |
| 65 | † | † |
| 66 | † | † |
| 67 | † | † |
| 68 | † | † |
| 69 | † | † |
| 70 | † | † |
| 71 | † | † |
| 72 | † | †† |
| 73 | † | † |
| 74 | † | † |
| 75 | † | † |
| 76 | † | † |
| 77 | † | † |
| 78 | † | † |
| 79 | † | † |
| 80 | † | † |
| 81 | † | † |
| 82 | † | † |
| 83 | † | † |
| 84 | † | † |
| 85 | † | †† |
| 86 | † | † |
| 87 | † | † |
| 88 | † | †† |
| 89 | † | † |
| 90 | † | † |
| 91 | † | † |
| 92 | † | † |
| 93 | † | † |
| 94 | † | † |
| 95 | † | † |
| 96 | † | † |
| 97 | † | † |
| 98 | † | † |
| 99 | † | † |
| 100 | † | † |
| 101 | † | † |
| 102 | † | †† |
| 103 | † | †† |
| 104 | † | † |
| 105 | † | † |
| 106 | †† | ††† |

### Example B: Caco2 Assay

Caco-2 cells are grown at 37° C. in an atmosphere of 5% CO₂ in DMEM growth medium supplemented with 10% (v/v) fetal bovine serum, 1% (v/v) nonessential amino acids, penicillin (100 U/mL), and streptomycin (100 μg/mL). Confluent cell monolayers are subcultured every 7 days or 4 days for Caco-2 by treatment with 0.05% trypsin containing 1 μM EDTA. Caco-2 cells are seeded in 96-well Transwell plates. The seeding density for Caco-2 cells is 14,000 cells/well. DMEM growth medium is replaced every other day after seeding. Cell monolayers are used for transport assays between 22 and 25 days for Caco-2 cells.

Cell culture medium is removed and replaced with HBSS. To measure the TEER, the HBSS is added into the donor compartment (apical side) and receiver compartment (basolateral side). The TEER is measured by using a REMS Autosampler to ensure the integrity of the cell monolayers. Caco-2 cell monolayers with TEER values 300 Q-cm² are used for transport experiments. To determine the Pₐₚₚ in the absorptive direction (A-B), solution of test compound (50 μM) in HBSS is added to the donor compartment (apical side), while HBSS solution with 4% BSA is added to the receiver compartment (basolateral side). The apical volume was 0.075 mL, and the basolateral volume is 0.25 mL. The incubation period is 120 min. at 37° C. in an atmosphere of 5% CO₂. At the end of the incubation period, samples from the donor and receiver sides are removed and an equal volume of MeCN is added for protein precipitation. The supernatants are collected after centrifugation (3000 rpm, Allegra X-14R Centrifuge from Beckman Coulter, Indianapolis, IN) for LCMS analysis. The permeability value is determined according to the equation:

$${P}_{app} ( cm / s ) = ( F * VD ) / ( SA * MD ) ,$$

where the flux rate (F, mass/time) is calculated from the slope of cumulative amounts of compound of interest on the receiver side, SA is the surface area of the cell membrane, VD is the donor volume, and MD is the initial amount of the solution in the donor chamber.

### Example C1: Luminescent Viability Assay

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) and NCI-H838 (KRAS WT; ATCC® CRL-5844) cells are cultured in RPMI 1640 media supplemented with 10% FBS (Gibco/Life Technologies). Eight hundred cells per well in RPMI 1640 media supplemented with 2% FBS are seeded into white, clear bottomed 384-well Costar tissue culture plates containing 50 nL dots of test compounds (final concentration is a 1:500 dilution, with a final concentration in 0.2% DMSO). Plates are incubated for 3 days at 37° C., 5% CO₂. At the end of the assay, 25 μL/well of CellTiter-Glo reagent (Promega) is added. Luminescence is read after 15 min. with a PHERAstar (BMG). Data are analyzed in Genedata Screener using SmartFit for IC₅₀ values.

### Example C2: Luminescent Viability Assay

NCI-H838 (KRAS WT; ATCC CRL-5844), BEN (KRAS WT; DSMZ ACC254), and SW620 (KRAS G12V; DSMZ ACC382) cells are cultured in media supplemented with 10% FBS. Eight hundred cells per well in media supplemented with 2% FBS are seeded into white, flat 384-well tissue culture microtiter plates containing 50 nL dots of test compounds (final concentration is a 1:500 dilution, with a final concentration in 0.2% DMSO). Plates are incubated for 3 days at 37° C., 5% CO₂. At the end of the assay, 25 μL/well of CellTiter-Glo reagent (Promega) is added. Luminescence is read after 10 min. with a PHERAstar (BMG). Data are analyzed in Genedata Screener using SmartFit for IC₅₀ values.

### Example D1: Cellular pERK HTRF Assay

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) and NCI-H838 (KRAS WT; ATCC® CRL-5844) cells are purchased from ATCC and maintained in RPMI 1640 media supplemented with 10% FBS (Gibco/Life Technologies). The cells are plated at 5000 cells per well (8 μL) into Greiner 384-well low volume, flat-bottom, and tissue culture treated white plates and incubated overnight at 37° C., 5% CO₂. The next morning, test compound stock solutions are diluted in media at 3× the final concentration and 4 μL are added to the cells, with a final concentration of 0.1% of DMSO. The cells are incubated with the test compounds for 4 h (G12C and G12V) or 2 h (G12D) at 37° C., 5% CO₂. 4 μL of 4× lysis buffer with blocking reagent (Cisbio) are added to each well and plates are rotated gently (300 rpm) for 30 min. at r.t. 4 μL per well of Cisbio anti Phospho-ERK 1/2 d2 is mixed with anti Phospho-ERK 1/2 Cryptate (1:1), and added to each well, incubated overnight in the dark at r.t. Plates are read on the Pherastar plate reader at 665 nm and 620 nm wavelengths. Data are analyzed in Genedata Screener using SmartFit for IC₅₀ values.

### Example D2: Cellular pERK HTRF Assay

NCI-H838 (KRAS WT; ATCC® CRL-5844), BEN (KRAS WT; DSMZ ACC254), and SW620 (KRAS G12V; DSMZ ACC382) cells are maintained in media supplemented with 10% FBS. The cells are plated at 5000 cells per well (8 μL) into 384-well low volume, flat-bottom, and tissue culture treated white plates and incubated overnight at 37° C., 5% CO₂. The next morning, test compound stock solutions are diluted in media at 3× the final concentration and 4 μL are added to the cells, with a final concentration of 0.1% of DMSO. The cells are incubated with the test compounds for 4 h at 37° C., 5% CO₂. 4 μL of 4× lysis buffer with blocking reagent Revvity are added to each well and plates are rotated gently (300 rpm) for 30 min. at r.t. 4 μL per well of anti Phospho-ERK 1/2 d2 is mixed with anti Phospho-ERK 1/2 Cryptate (1:1), and added to each well, incubated overnight in the dark at r.t. Plates are read on the Pherastar plate reader at 665 nm and 620 nm wavelengths. Data are analyzed in Genedata Screener using SmartFit for IC₅₀ values.

### Example E1: Whole Blood pERK1/2 HTRF Assay

MIA PaCa-2 cells (KRAS G12C; ATCC® CRL-1420), HPAF-II (KRAS G12D; ATCC® CRL-1997) and YAPC (KRAS G12V; DSMZ ACC382) are maintained in RPMI 1640 with 10% FBS (Gibco/Life Technologies). For MIA PaCa-2 assay, cells are seeded into 96 well tissue culture plates (Corning #3596) at 25000 cells per well in 100 μL media and cultured for 2 days at 37° C., 5% CO₂ before the assay. For HPAF-II and YAPC assay, cells are seeded in 96 well tissue culture plates at 50000 cells per well in 100 μL media and cultured for 1 day before the assay. Whole Blood are added to the 1 μL dots of compounds (prepared in DMSO) in 96 well plates and mixed gently by pipetting up and down so that the concentration of the compound in blood is 1× of desired concentration, in 0.5% DMSO. The media is aspirated from the cells and 50 μL per well of whole blood with test compound is added and incubated for 4 h for MIA PaCa and YAPC assay; or 2 h for HPAF-II assay, respectively at 37° C., 5% CO₂. After dumping the blood, the plates are gently washed twice by adding PBS to the side of the wells and dumping the PBS from the plate onto a paper towel, tapping the plate to drain well. 50 μL/well of 1× lysis buffer #1 (Cisbio) with blocking reagent (Cisbio) and Benzonase nuclease (Sigma Cat #E1014-5KU, 1: 10000 final concentration) is then added and incubated at r.t. for 30 min. with shaking (250 rpm). Following lysis, 16 μL of lysate is transferred into 384-well Greiner small volume white plate using an Assist Plus (Integra Biosciences, NH). 4 μL of 1:1 mixture of anti Phospho-ERK 1/2 d2 and anti Phospho-ERK 1/2 Cryptate (Cisbio) is added to the wells using the Assist Plus and incubated at r.t. overnight in the dark. Plates are read on the Pherastar plate reader at 665 nm and 620 nm wavelengths. Data are analyzed in Genedata Screener using SmartFit for IC₅₀ values.

### Example E2: Whole Blood pERK1/2 HTRF Assay

BEN (KRAS WT; DSMZ ACC254), and YAPC (KRAS G12V; DSMZ ACC382) are maintained in media supplemented with 10% FBS. Cells are seeded in 96 well tissue culture plates at 50000 cells per well in 100 μL media and cultured for 1 day before the assay. Whole Blood is added to the 1 μL dots of compounds (prepared in DMSO) in 96 well plates and mixed gently by pipetting up and down so that the concentration of the compound in blood is 1× of desired concentration, in 0.6% DMSO. The media is aspirated from the cells and 50 μL per well of whole blood with test compound is added and incubated for 4 h at 37° C., 5% CO₂. After the 4 h incubation, the plates are washed once in PBS using the centrifugal Blue Washer (BlueCat Bio) with PBS. 50 μL/well of 1× lysis buffer (Revvity) with blocking reagent (Revvity) and Benzonase nuclease (1:10000 dilution Sigma) is then added and incubated at r.t. for 30 min. with shaking (250 rpm). Following lysis, 16 μL of lysate is transferred into 384-well small volume white plate (Greiner) using an Assist Plus (Integra Biosciences, NH). 4 μL of 1:1 mixture of anti Phospho-ERK 1/2 d2 and anti Phospho-ERK 1/2 Cryptate (Revvity) is added to the wells using the Assist Plus and incubated at r.t. overnight in the dark. Plates are read on the Pherastar plate reader at 665 nm and 620 nm wavelengths. Data are analyzed in Genedata Screener using SmartFit for IC₅₀ values.

### Example F: Ras Activation Elisa

The 96-Well Ras Activation ELISA Kit (Cell Biolabs Inc; #STA441) uses the Raf1 RBD (Rho binding domain) bound to a 96-well plate to selectively pull down the active form of Ras from cell lysates. The captured GTP-Ras is then detected by a pan-Ras antibody and HRP-conjugated secondary antibody.

MIA PaCa-2 (KRAS G12C; ATCC® CRL-1420), NCI-H358 (KRAS G12C; ATCC® CRL-5807), A427 (KRAS G12D; ATCC® HTB53), HPAFII (KRAS G12D; ATCC® CRL-1997), YAPC (KRAS G12V; DSMZ ACC382), SW480 (KRAS G12V; ATCC® CRL-228) and NCI-H838 (KRAS WT; ATCC® CRL-5844) cells are maintained in RPMI 1640 with 10% FBS (Gibco/Life Technologies). The cells are seeded into 96 well tissue culture plates (Corning #3596) at 25000 cells per well in 100 μL media and cultured for 2 days at 37° C., 5% CO₂ so that they are approximately 80% confluent at the start of the assay. The cells are treated with compounds for either 4 h or overnight at 37° C., 5% CO₂. At the time of harvesting, the cells are washed with PBS, drained well and then lysed with 50 μL of the 1× Lysis buffer (provided by the kit) plus added Halt Protease and Phosphatase inhibitors (1:100) for 1 h on ice.

The Raf-1 RBD is diluted 1:500 in Assay Diluent (provided in kit) and 100 μL of the diluted Raf-1 RBD is added to each well of the Raf-1 RBD Capture Plate. The plate is covered with a plate sealing film and incubated at r.t. for 1 h on an orbital shaker. The plate is washed 3 times with 250 μL 1× Wash Buffer per well with thorough aspiration between each wash. 50 μL of Ras lysate sample (10-100 μg) is added per well in duplicate. A “no cell lysate” control is added in a couple of wells for background determination. 50 μL of Assay Diluent is added to all wells immediately to each well and the plate is incubated at r.t. for 1 h on an orbital shaker. The plate is washed 5 times with 250 μL 1× Wash Buffer per well with thorough aspiration between each wash. 100 μL of the diluted Anti-pan-Ras Antibody is added to each well and the plate is incubated at r.t. for 1 h on an orbital shaker. The plate is washed 5 times as previously. 100 μL of the diluted Secondary Antibody, HRP Conjugate is added to each well and the plate is incubated at r.t. for 1 h on an orbital shaker. The plate is washed 5 times as previously and drained well. 100 L of Chemiluminescent Reagent (provided in the kit) is added to each well, including the blank wells. The plate is incubated at r.t. for 5 min. on an orbital shaker before the luminescence of each microwell is read on a plate luminometer. The % inhibition is calculated relative to the DMSO control wells after a background level of the “no lysate control” is subtracted from all the values. IC₅₀ determination is performed by fitting the curve of inhibitor percent inhibition versus the log of the inhibitor concentration using the GraphPad Prism 7 software.

### Example G: Inhibition of RAS-RAF and P13K-AKT Pathways

The cellular potency of compounds is determined by measuring phosphorylation of KRAS downstream effectors extracellular-signal-regulated kinase (ERK), ribosomal S6 kinase (RSK), AKT (also known as protein kinase B, PKB) and downstream substrate S6 ribosomal protein.

To measure phosphorylated extracellular-signal-regulated kinase (ERK), ribosomal S6 kinase (RSK), AKT and S6 ribosomal protein, cells (details regarding the cell lines and types of data produced are further detailed in Table B) are seeded overnight in Corning 96-well tissue culture treated plates in RPMI medium with 10% FBS at 4×104 cells/well. The following day, cells are incubated in the presence or absence of a concentration range of test compounds for 4 h at 37° C., 5% CO₂. Cells are washed with PBS and lysed with 1× lysis buffer (Cisbio) with protease and phosphatase inhibitors (Thermo Fisher, 78446). 10 or 20 μg of total protein lysates is subjected to SDS-PAGE and immunoblot analysis using following antibodies: phospho-ERK1/2-Thr202/Tyr204 (#9101 L), total-ERK1/2 (#9102L), phosphor-AKT-Ser473 (#4060L), phospho-p90RSK-Ser380 (#11989S) and phospho-S6 ribosomal protein-Ser235/Ser236 (#2211S) are from Cell Signaling Technologies (Danvers, MA).

TABLE B

| | | KRAS | |
|---|---|---|---|
| Cell Line | Histology | alteration | Readout |
| H358 | Lung | G12C | pERK, pAKT, p-S6, p-p90RSK |
| MIA PaCa-2 | Pancreas | G12C | pERK, pAKT, p-S6, p-p90RSK |
| HPAF II | Pancreas | G12D | pERK, pAKT, p-S6, p-p90RSK |
| A427 | Lung | G12D | pERK, pAKT, p-S6, p-p90RSK |
| AGS | Stomach | G12D | pERK, pAKT, p-S6, p-p90RSK |
| PaTu 8988s | Pancreas | G12V | pERK, pAKT, p-S6, p-p90RSK |
| H441 | Lung | G12V | pERK, pAKT, p-S6, p-p90RSK |
| YAPC | Pancreas | G12V | pERK, pAKT, p-S6, p-p90RSK |
| SW480 | Colorectal | G12V | pERK, pAKT, p-S6, p-p90RSK |

### Example H1: In Vivo Efficacy Studies

MIA-PaCa-2 (KRAS G12C), H358 (KRAS G12C), HPAF-II (KRAS G12D), AGS (KRAS G12D), SW480 (KRAS G12V) or YAPC(KRAS G12V) human cancer cells are obtained from the American Type Culture Collection and maintained in RPMI media supplemented with 10% FBS. For efficacy studies experiments, 5×106 cells are inoculated subcutaneously into the right hind flank of 6- to 8-week-old BALB/c nude mice (Charles River Laboratories, Wilmington, MA, USA). When tumor volumes are approximately 150-250 mm3, mice are randomized by tumor volume and compounds are orally administered. Tumor volume is calculated using the formula (L×W2)/2, where L and W refer to the length and width dimensions, respectively. Tumor growth inhibition is calculated using the formula (1−(VT/VC))×100, where VT is the tumor volume of the treatment group on the last day of treatment, and VC is the tumor volume of the control group on the last day of treatment. Two-way analysis of variance with Dunnett's multiple comparisons test is used to determine statistical differences between treatment groups (GraphPad Prism). Mice are housed at 10-12 animals per cage, and are provided enrichment and exposed to 12 h light/dark cycles. Mice whose tumor volumes exceeded limits (10% of body weight) are humanely euthanized by CO₂ inhalation. Animals are maintained in a barrier facility fully accredited by the Association for Assessment and Accreditation of Laboratory Animal Care, International. All of the procedures are conducted in accordance with the US Public Service Policy on Human Care and Use of Laboratory Animals and with Incyte Animal Care and Use Committee Guidelines.

### Example H2: In Vivo Efficacy Studies

MIA-PaCa-2 (KRAS G12C), H358 (KRAS G12C), HPAF-II (KRAS G12D), AGS (KRAS G12D), SW480 (KRAS G12V), SW620(KRAS G12V) or YAPC(KRAS G12V) human cancer cells are obtained from the American Type Culture Collection and maintained in RPMI media supplemented with 10% FBS. For efficacy studies experiments, 5×10⁶ cells are inoculated subcutaneously into the right hind flank of 6- to 8-week-old NCr nude mice (Taconic Biosciences, Rensselear, NY, USA). When tumor volumes are approximately 150-300250 mm3, mice are randomized by tumor volume and compounds are orally administered. Tumor volume is calculated using the formula (L×W²)/2, where L and W refer to the length and width dimensions, respectively. Tumor growth inhibition is calculated using the formula (1-(V_{T}/V_{C}))×100, where V_{T} is the average tumor volume of the treatment group on the last day of treatment, and V_{C} is the average tumor volume of the control group on the last day of treatment. Two-way analysis of variance with Dunnett's multiple comparisons test is used to determine statistical differences between treatment groups (GraphPad Prism). Mice are housed at 10-12 animals per cage, and are provided enrichment and exposed to 12 h light/dark cycles. Mice whose tumor volumes exceed limits (10% of body weight) for two consecutive measurements are humanely euthanized by CO₂ inhalation. Animals are maintained in a barrier facility fully accredited by the Association for Assessment and Accreditation of Laboratory Animal Care, International. All of the procedures are conducted in accordance with the US Public Service Policy on Human Care and Use of Laboratory Animals and with Incyte Animal Care and Use Committee Guidelines.

### Example I: Human Whole Blood Stability

The whole blood stability of the exemplified compounds is determined by LCMS/MS. The 96-Well Flexi-Tier™ Block (Analytical Sales & Services, Inc, Flanders, NJ) is used for the incubation plate containing 1.0 mL glass vials with 0.5 mL of blood per vial (pooled gender, human whole blood sourced from BIOIVT, Hicksville, NY or similar). Blood is pre-warmed in water bath to 37° C. for 30 min. A 96-deep well analysis plate is prepared with the addition of 100 μL ultrapure water/well. 50 μL chilled ultrapure water/well is added to 96-deep well sample collection plate and covered with a sealing mat. 1 μL of 0.5 M compound working solution (DMSO:water) is added to the blood in incubation plate to reach final concentrations of 1 μM, mixed by pipetting thoroughly and 50 μL is transferred 50 into the T=0 wells of the sample collection plate. Blood is allowed to sit in the water for 2 min. and then 400 μL stop solution/well is added (MeCN containing an internal standard). The incubation plate is placed in the Incu-Shaker CO₂ Mini incubator (Benchmark Scientific, Sayreville, NJ) at 37° C. with shaking at 150 rpm. At 1, 2 and 4 h, the blood samples are mixed thoroughly by pipetting and 50 μL is transferred into the corresponding wells of the sample collection plate. Blood is allowed to sit in the water for 2 min. and then 400 μL of stop solution/well is added. The collection plate is sealed and vortexed at 1700 rpm for 3 min. (VX-2500 Multi-Tube Vortexer, VWR International, Radnor, PA), and samples are then centrifuged in the collection plate at 3500 rpm for 10 min. (Allegra X-14R Centrifuge Beckman Coulter, Indianapolis, IN). 100 μL of supernatant/well is transferred from the sample collection plate into the corresponding wells of the analysis plate. The final plate is vortexed at 1700 rpm for 1 min. and analyze samples by LCMS/MS. The peak area ratio of the 1, 2, and 4 h samples relative to T=0 is used to determine the percent remaining. The natural log of the percent remaining versus time is used determine a slope to calculate the compounds half-life in blood (t_{1/2}=0.693/slope).

### Example J: In Vitro Intrinsic Clearance Protocol

For in vitro metabolic stability experiments, test compounds are incubated with human liver microsomes at 37° C. The incubation mixture contains test compounds (1 μM), NADPH (2 mM), and human liver microsomes (0.5 mg protein/mL) in 100 mM phosphate buffer (pH 7.4). The mixture is pre-incubated for 2 min. at 37° C. before the addition of NADPH. Reactions are commenced upon the addition of NADPH and quenched with ice-cold MeOH at 0, 10, 20, and 30 min. Terminated incubation mixtures are analyzed using LCMS/MS system. The analytical system consisted of a Shimadzu LC-30AD binary pump system and SIL-30AC autosampler (Shimadzu Scientific Instruments, Columbia, MD) coupled with a Sciex Triple Quad 6500+mass spectrometer from Applied Biosystems (Foster City, CA). Chromatographic separation of test compounds and internal standard is achieved using a Hypersil Gold C₁₈ column (50×2.1 mm, 5 μM, 175 Å) from ThermoFisher Scientific (Waltham, MA). Mobile phase A consists of 0.1% formic acid in water, and mobile phase B consists of 0.1% formic acid in MeCN. The total LCMS/MS runtime can be 2.75 min. with a flow rate of 0.75 mL/min. Peak area integrations and peak area ratio calculations are performed using Analyst software (version 1.6.3) from Applied Biosystems.

The in vitro intrinsic clearance, CL_{int, in vitro}, is calculated from the t_{1/2} of test compound disappearance as CL_{int, in vitro}=(0.693/t_{1/2})×(1/Cₚᵣₒₜₑᵢₙ), where Cₚᵣₒₜₑᵢₙ is the protein concentration during the incubation, and t_{1/2} is determined by the slope (k) of the log-linear regression analysis of the concentration versus time profiles; thus, t_{1/2}=ln2/k. The CL_{int, in vitro} values are scaled to the in vivo values for human by using physiologically based scaling factors, hepatic microsomal protein concentrations (45 mg protein/g liver), and liver weights (21 μg/kg body weight). The equation CLᵢₙₜ=CL_{int, in vitro}×(mg protein/g liver weight)×(g liver weight/kg body weight) is used. The in vivo hepatic clearance (CL_{H}) is then calculated by using CLᵢₙₜ and hepatic blood flow, Q (20 mL·min.⁻¹·kg⁻¹ in humans) in the well-stirred liver model disregarding all binding from CL_{H}=(Q×CLᵢₙₜ)/(Q+CLᵢₙₜ). The hepatic extraction ratio was calculated as CL_{H} divided by Q.

### Example K: In Vivo Pharmacokinetics Protocol

For in vivo pharmacokinetic experiments, test compounds are administered to male Sprague Dawley rats or male and female Cynomolgus monkeys intravenously or via oral gavage. For intravenous (IV) dosing, test compounds are dosed at 0.5 to 1 mg/kg using a formulation of 10% dimethylacetamide (DMAC) in acidified saline via IV bolus for rat and 5 min. or 10 min. IV infusion for monkey. For oral (PO) dosing, test compounds are dosed at 1.0 to 3.0 mg/kg using 5% DMAC in 0.5% methylcellulose in citrate buffer (pH 2.5). Blood samples are collected at predose and various time points up to 24 h postdose. All blood samples are collected using EDTA as the anticoagulant and centrifuged to obtain plasma samples. The plasma concentrations of test compounds are determined by LCMS methods. The measured plasma concentrations are used to calculate PK parameters by standard noncompartmental methods using Phoenix® WinNonlin software program (version 8.0, Pharsight Corporation).

In rats and monkeys, cassette dosing of test compounds are conducted to obtain preliminary PK parameters.

In vivo pharmacokinetic experiments with male beagle dogs may be performed under the conditions described above.

### Example L: Time Dependent Inhibition (TDI) of CYP Protocol

This assay is designed to characterize an increase in CYP inhibition as a test compounds is metabolized over time. Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CYP inhibitor could result in a positive result.

The results are from a cocktail of CYP specific probe substrates at 4 times their Km concentrations for CYP2C9, 2C19, 2D6 and 3A4 (midazolam) using human liver microsomes (HLM). The HLMs can be pre-incubated with test compounds at a concentration 10 μM for 30 min. in the presence (+N) or absence (−N) of a NADPH regenerating system, diluted 10-fold, and incubated for 8.min. in the presence of the substrate cocktail with the addition of a fresh aliquot of NADPH regenerating system. A calibration curve of metabolite standards can be used to quantitatively measure the enzyme activity using LCMS/MS. In addition, incubations with known time dependent inhibitors, tienilic aicd (CYP2C9), ticlopidine (CYP2C19), paroxetine (CYP2D6), and troleandomycin (CYP3A4), used as positive controls are pre-incubated 30 min. with or without a NADPH regenerating system.

The analytical system consists of a Shimadzu LC-30AD binary pump system and SIL-30AC autosampler (Shimadzu Scientific Instruments, Columbia, MD) coupled with a Sciex Triple Quad 6500+mass spectrometer from Applied Biosystems (Foster City, CA). Chromatographic separation of test compounds and internal standard can be achieved using an ACQUITY UPLC BEH 130A, 2.1×50 mm, 1.7 μm HPLC column (Waters Corp, Milford, MA). Mobile phase A consists of 0.1% formic acid in water, and mobile phase B consists of 0.1% formic acid in MeCN. The total LCMS/MS runtime will be 2.50 min. with a flow rate of 0.9 mL/min. Peak area integrations and peak area ratio calculations are performed using Analyst software (version 1.6.3) from Applied Biosystems.

The percentage of control CYP2C9, CYP2C19, CYP2D6, and CYP3A4 activity remaining following preincubation of the compounds with NADPH is corrected for the corresponding control vehicle activity and then calculated based on 0 min. as 100%. A linear regression plot of the natural log of % activity remaining versus time for each isozyme is used to calculate the slope. The -slope is equal to the rate of enzyme loss, or the K_{obs}.

Various modifications of the present disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound of Formula (I): 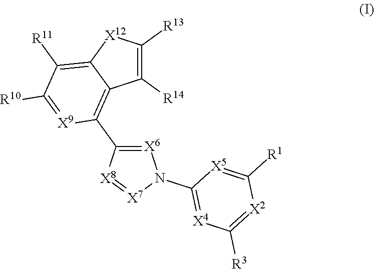
or a pharmaceutically acceptable salt thereof;
wherein
R¹ is selected from H, D, C₁₋₃ alkyl, C₁₋₃ haloalkyl, C₂₋₃ alkenyl, C₂₋₃ alkynyl, C₃₋₅ cycloalkyl, 4-10 membered heterocycloalkyl, phenyl, 5-10 membered heteroaryl, C₃₋₆ cycloalkyl-C₁₋₃ alkylene, 4-10 membered heterocycloalkyl-C₁₋₃ alkylene, phenyl-C₁₋₃ alkylene, 5-10 membered heteroaryl-C₁₋₃alkylene, halo, CN, OR^{a1}, C(O)R^{b1}, C(O)NR^{c1}R^{d1}, NR^{c1}R^{e1}, and NR^{c1}C(O)R^{b1}; wherein the C₃₋₅cycloalkyl, 4-10 membered heterocycloalkyl, phenyl, 5-10 membered heteroaryl, C₃₋₆cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene forming R¹ are each optionally substituted with 1, 2, or 3 substituents independently selected from R^{1A}; and wherein the C₁₋₃ alkyl, C₂₋₃ alkenyl, and C₂₋₃ alkynyl forming R¹ are each optionally substituted with 1, 2, or 3 substituents independently selected from R^{1B};
each R^{1A} is independently selected from C₁₋₃ alkyl, C₁₋₃ haloalkyl, C₂₋₃ alkenyl, C₂₋₃ alkynyl, and R^{1B}; wherein the C₁₋₃ alkyl, C₂₋₃ alkenyl, and C₂₋₃ alkynyl forming R^{1A} are each optionally substituted with 1, 2, or 3 substituents each independently selected from R^{1B};
each R^{1B} is independently selected from D, C₃₋₆ cycloalkyl, 4-10 membered heterocycloalkyl, phenyl, 5-6 membered heteroaryl, halo, CN, OR^{a1B}, C(O)R^{b1B}, NR^{c1B}C(O)R^{b1B}, C(O)NR^{c1B}R^{d1B}, C(O)OR^{a1B}, NR^{c1B}R^{d1B}, and S(O)₂R^{b1B}; wherein the C₃₋₆-cycloalkyl, 4-6 membered heterocycloalkyl, phenyl, and 5-6 membered heteroaryl forming R^{1B} are each optionally substituted with 1, 2, or 3 substituents independently selected from R^{1C};
each R^{1C} is independently selected from D, C₃₋₆cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C}; wherein the C₃₋₆cycloalkyl, 4-6 membered heterocycloalkyl, phenyl, and 5-6 membered heteroaryl forming R^{1C} are each optionally substituted with 1, 2, or 3 substituents independently selected from R^{1D};
each R^{1D} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a1D}, C(O)R^{b1D}, C(O)NR^{c1D}R^{d1D}, C(O)OR^{a1D}, NR^{c1D}R^{d1D}, and S(O)₂R^{b1D};
each R^{a1} is independently selected from C₁₋₆ alkyl, C₁₋₃ haloalkyl, C₂₋₆ alkenyl, C₂₋₆ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a1} are each optionally substituted with 1, 2, or 3 substituents independently selected from R^{1A}; wherein the C₁₋₆ alkyl, C₂₋₆ alkenyl, and C₂₋₆ alkynyl forming R^{a1} are each optionally substituted with 1, 2, or 3 substituents independently selected from R^{1B};
each R^{b1}, R^{c1}, and R^{d1} is independently selected from H, C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; and wherein the C₃₋₆ cycloalkyl, 4-6 membered heterocycloalkyl, phenyl, and 5-6 membered heteroaryl forming R^{b1}, R^{c1}, and R^{d1} are each optionally substituted with 1, 2, or 3 substituents independently selected from R^{1A}; wherein the C₁₋₃ alkyl, C₁₋₃ haloalkyl, C₂₋₃ alkenyl, and C₂₋₃ alkynyl, forming R^{b1}, R^{c1}, and R^{d1} are each optionally substituted with 1, 2, or 3 substituents independently selected from R^{1B}; or
any R^{c1} and R^{d1} attached to the same N atom, together with the N atom to which they are attached, form a 4-, 5-, or 6-membered heterocycloalkyl group optionally substituted with 1, 2, or 3 substituents independently selected from R^{1A},
each R^{e1} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{e1} are each optionally substituted with 1, 2, or 3 substituents independently selected from R^{1A}; wherein the C₁₋₃ alkyl, C₁₋₃ haloalkyl, C₂₋₃ alkenyl, and C₂₋₃ alkynyl forming R^{e1} are each optionally substituted with 1, 2, or 3 substituents independently selected from R^{1B}; or
R^{c1} and R^{e1} attached to the same N atom, together with the N atom to which they are attached, form a 4-, 5-, or 6-membered heterocycloalkyl group optionally substituted with 1, 2, or 3 substituents independently selected from R^{1A},
each R^{a1B}, R^{b1B}, R^{c1B}, and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1C}, R^{b1C}, R^{c1C}, and R^{d1C} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1D}, R^{b1D}, R^{c1D}, and R^{d1D} is independently selected from H, C₁₋₃ alkyl, and C₁₋₃ haloalkyl;
X² is N or CR²;
R² is selected from H, C₁₋₃ alkyl, C₁₋₃ haloalkyl, C₂₋₃ alkenyl, C₂₋₃ alkynyl, C₃₋₁₀cycloalkyl, 4-10 membered heterocycloalkyl, halo, CN, OR^{2A}, and NR^{2B}R^{2C}; wherein the C₃₋₁₀ cycloalkyl and 4-10 membered heterocycloalkyl forming R² are each optionally substituted with 1, 2, or 3 substituents independently selected from R^{2D}; and wherein the C₁₋₃ alkyl, C₂₋₃ alkenyl, and C₂₋₃ alkynyl forming R² are each optionally substituted with 1, 2, or 3 substituents independently selected from R^{2E};
R^{2A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{2B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{2C} is selected from H, C₁₋₃ alkyl, C₂₋₃ alkenyl, and C₂₋₃ alkynyl; or
R^{2B} and R^{2C}, together with the N atom to which they are both attached, optionally form a 4-, 5-, 6-, or 7-membered heterocycloalkyl group that is optionally substituted with 1, 2, or 3 substituents independently selected from independently selected from R^{2D};
each R^{2D} is independently selected from C₁₋₃ alkyl, C₁₋₃ haloalkyl, C₂₋₃ alkenyl, and C₂₋₃ alkynyl;
each R^{2E} is independently selected from D, halo, CN, OH, NH₂, NH(C₁₋₃ alkyl), and N(C₁₋₃ alkyl)₂;
R³ is selected from H, D, C₁₋₃ alkyl, C₁₋₃ haloalkyl, C₂₋₃ alkenyl, C₂₋₃ alkynyl, C₃₋₅ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a3}, C(O)R¹³, C(O)NR^{c3}R^{d3}, NR^{c3}R^{e3}, and NR^{c3}C(O)R^{b3}; wherein the C₃₋₅ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, and 5-6 membered heteroaryl-C₁₋₃ alkylene forming R³ are each optionally substituted with 1, 2, or 3 substituents independently selected from R^{3A}; wherein the C₁₋₃ alkyl, C₂₋₃ alkenyl, and C₂₋₃ alkynyl forming R³ are each optionally substituted with 1, 2, or 3 substituents independently selected from R^{3B};
each R^{3A} is independently selected from C₁₋₃ alkyl, C₁₋₃ haloalkyl, C₂₋₃ alkenyl, C₂₋₃ alkynyl, and R^{3B}; wherein the C₁₋₃ alkyl, C₂₋₃ alkenyl, and C₂₋₃ alkynyl forming R^{3A} are each optionally substituted with 1, 2, or 3 substituents each independently selected from R^{3B};
each R^{3B} is independently selected from D, C₃₋₆ cycloalkyl, 4-10 membered heterocycloalkyl, phenyl, 5-6 membered heteroaryl, halo, CN, C(O)R^{b3B}, C(O)NR^{c3B}R^{d3B}, NR^{c3B}C(O)R^{b3B}, C(O)OR^{a3B}, NR^{c3B}R^{d3B}, and S(O)₂R^{b3B}; wherein the C₃₋₆cycloalkyl, 4-6 membered heterocycloalkyl, phenyl, and 5-6 membered heteroaryl forming R^{3B} are each optionally substituted with 1, 2, or 3 substituents independently selected from R^{3C};
each R^{3C} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a3C}, C(O)R^{b3C}, C(O)NR^{c3C}R^{d3C}, C(O)OR^{a3C}, NR^{c3C}R^{d3C}, and S(O)₂R^{b3C};
each R^{a3} is independently selected from C₁₋₆ alkyl, C₁₋₃ haloalkyl, C₂₋₆ alkenyl, C₂₋₆ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{a3} are each optionally substituted with 1, 2, or 3 substituents independently selected from R^{3A}; wherein the C₁₋₆ alkyl, C₂₋₆ alkenyl, and C₂₋₆ alkynyl forming R^{a3} are each optionally substituted with 1, 2, or 3 substituents independently selected from R^{3B};
each R^{b3}, R^{c3}, and R^{d3} is independently selected from H, C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; and wherein the C₃₋₆ cycloalkyl, 4-6 membered heterocycloalkyl, phenyl, and 5-6 membered heteroaryl forming R^{b3}, R^{c3}, and R^{d3} are each optionally substituted with 1, 2, or 3 substituents independently selected from R^{3A}; wherein the C₁₋₃ alkyl, C₁₋₃ haloalkyl, C₂₋₃ alkenyl, and C₂₋₃ alkynyl, forming R^{b3}, R^{c3}, and R^{d3} are each optionally substituted with 1, 2, or 3 substituents independently selected from R^{3B}; or
any R^{c3} and R^{d3} attached to the same N atom, together with the N atom to which they are attached, form a 4-, 5-, or 6-membered heterocycloalkyl group optionally substituted with 1, 2, or 3 substituents independently selected from R^{3A};
each R^{e3} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, and 5-6 membered heteroaryl; wherein the C₃₋₆ cycloalkyl, 4-6 membered heterocycloalkyl, phenyl, and 5-6 membered heteroaryl forming R^{e3} are each optionally substituted with 1, 2, or 3 substituents independently selected from R^{3A}; wherein the C₁₋₃ alkyl, C₁₋₃ haloalkyl, C₂₋₃ alkenyl, and C₂₋₃ alkynyl forming R^{e3} are each optionally substituted with 1, 2, or 3 substituents independently selected from R^{3B}; or
R^{c3} and R^{e3} attached to the same N atom, together with the N atom to which they are attached, form a 4-, 5-, or 6-membered heterocycloalkyl group optionally substituted with 1, 2, or 3 substituents independently selected from R^{3A},
each R^{a3B}, R^{b3B}, R^{c3B}, and R^{d3B} is independently selected from H, C₁₋₃ alkyl, and C₁₋₃ haloalkyl;
each R^{a3}, R^{b3C}, R^{c3C}, and R^{d3C} is independently selected from H, C₁₋₃ alkyl, and C₁₋₃ haloalkyl;
X⁴ is N or CR⁴;
R⁴ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, halo, CN, OR^{4A}, and NR^{4B}R^{4C}; wherein the C₃₋₁₀ cycloalkyl and 4-10 membered heterocycloalkyl forming R⁴ are each optionally substituted with 1, 2, or 3 substituents independently selected from R^{4D}; and wherein the C₁₋₃ alkyl, C₂₋₃ alkenyl, and C₂₋₃ alkynyl forming R⁴ are each optionally substituted with 1, 2, or 3 substituents independently selected from R^{4E};
R^{4A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{4B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{4C} is selected from H, C₁₋₃ alkyl, C₂₋₃ alkenyl, and C₂₋₃ alkynyl; or
R^{4B} and R^{4C}, together with the N atom to which they are both attached, optionally form a 4-, 5-, 6-, or 7-membered heterocycloalkyl group that is optionally substituted with 1, 2, or 3 substituents independently selected from independently selected from R^{4D};
each R^{4D} is independently selected from C₁₋₃ alkyl, C₁₋₃ haloalkyl, C₂₋₃ alkenyl, and C₂₋₃ alkynyl;
each R^{4E} is independently selected from D, halo, CN, OH, NH₂, NH(C₁₋₃ alkyl), and N(C₁₋₃ alkyl)₂;
X⁵ is N or CR⁵;
R⁵ is selected from H, C₁₋₃ alkyl, C₁₋₃ haloalkyl, C₂₋₃ alkenyl, C₂₋₃ alkynyl, C₃₋₁₀cycloalkyl, 4-10 membered heterocycloalkyl, halo, CN, OR^{SA}, and NR^{5B}R^{5C}; wherein the C₃₋₁₀ cycloalkyl and 4-10 membered heterocycloalkyl forming R⁵ are each optionally substituted with 1, 2, or 3 substituents independently selected from R^{5D}; and wherein the C₁₋₃ alkyl, C₂₋₃ alkenyl, and C₂₋₃ alkynyl forming R⁵ are each optionally substituted with 1, 2, or 3 substituents independently selected from R^{5E};
R^{5A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{5B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{5C} is selected from H, C₁₋₃ alkyl, C₂₋₃ alkenyl, and C₂₋₃ alkynyl; or
R^{5B} and R^{5C}, together with the N atom to which they are both attached, optionally form a 4-, 5-, 6-, or 7-membered heterocycloalkyl group that is optionally substituted with 1, 2, or 3 substituents independently selected from independently selected from R^{5D};
each R^{5D} is independently selected from C₁₋₃ alkyl, C₁₋₃ haloalkyl, C₂₋₃ alkenyl, and C₂₋₃ alkynyl;
each R^{5E} is independently selected from D, halo, CN, OH, NH₂, NH(C₁₋₃ alkyl), and N(C₁₋₃ alkyl)₂;
X⁶ is N or CR⁶;
R⁶ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, CN, OR^{6A}, and NR^{6B}R^{6C}; wherein the C₃₋₁₀ cycloalkyl, 4-10 membered heterocycloalkyl, C₆₋₁₀ aryl, and 5-10 membered heteroaryl forming R⁶ are each optionally substituted with 1, 2, or 3 substituents independently selected from R^{6D}; wherein the C₁₋₃ alkyl, C₂₋₃ alkenyl, and C₂₋₃ alkynyl forming R⁶ are each optionally substituted with 1, 2, or 3 substituents independently selected from R^{6E};
R^{6A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{6A} are each optionally substituted with 1, 2, or 3 substituents independently selected from R^{6D}; wherein the C₁₋₃ alkyl, C₂₋₃ alkenyl, and C₂₋₃ alkynyl forming R^{6A} are each optionally substituted with 1, 2, or 3 substituents independently selected from R^{6E};
R^{6B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{6B} are each optionally substituted with 1, 2, or 3 substituents independently selected from R^{6D}; wherein the C₁₋₃ alkyl, C₂₋₃ alkenyl, and C₂₋₃ alkynyl forming R^{6B} are each optionally substituted with 1, 2, or 3 substituents independently selected from R^{6E};
R^{6C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{6C} are each optionally substituted with 1, 2, or 3 substituents independently selected from R^{6E}; or
R^{6B} and R^{6C}, together with the N atom to which they are both attached, optionally form a 4-, 5-, 6-, or 7-membered heterocycloalkyl group that is optionally substituted with 1, 2, or 3 substituents independently selected from independently selected from R^{6D};
each R^{6D} is independently selected from C₁₋₃ alkyl, C₁₋₃ haloalkyl, C₂₋₃ alkenyl, C₂₋₃ alkynyl, and R^{6E}; wherein each of the C₁₋₃ alkyl, C₂₋₃ alkenyl, and C₂₋₃ alkynyl forming R^{6D} is optionally substituted with 1, 2, or 3 substituents independently selected from R^{6E};
each R^{6E} is independently selected from D, halo, CN, OR^{a6}, SR^{a6}, C(O)R^{b6}, C(O)NR^{c6}R^{d6}, C(O)OR^{a6}, OC(O)R^{b6}, OC(O)NR^{c6}R^{d6}, NR^{c6}R^{d6}, NR^{c6}C(O)R^{b6}, NR^{c6}C(O)NR^{c6}R^{d6}, NR^{c6}C(O)OR^{a6}, C(═NR^{e6})NR^{c6}R^{d6}, NR^{c6}C(═NR^{e6})NR^{c6}R^{d6}, S(O)R^{b6}, S(O)NR^{c6}R^{d6}, S(O)₂R^{b6}, NR^{c6}S(O)₂R^{b6}, and S(O)₂NR^{c6}R^{d6};
R^{a6}, R^{b6}, R^{c6}, and R^{d6} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a6}, R^{b6}, R^{c6}, and R^{d6} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{b6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6A}, C(═NR^{e6A})NR^{c6A}R^{d6A}, NR^{c6A}C(═NR^{e6A})NR^{c6A}R^{d6A} S(O)R^{b6A}, S(O)NR^{c6A}R^{d6A}, S(O)₂R^{b6A}, NR^{c6A}S(O)₂R^{b6A}, and S(O)₂NR^{c6A}R^{d6A}; or
R^{c6} and R^{d6} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a6A}, SR^{a6A}, C(O)R^{b6A}, C(O)NR^{c6A}R^{d6A}, C(O)OR^{a6A}, OC(O)R^{b6A}, OC(O)NR^{c6A}R^{d6A}, NR^{c6A}R^{d6A}, NR^{c6A}C(O)R^{b6A}, NR^{c6A}C(O)NR^{c6A}R^{d6A}, NR^{c6A}C(O)OR^{a6A}, C(═NR^{e6A})NR^{c6A}R^{d6A}, NR^{c6A}C(═NR^{e6A})NR^{c6A}R^{d6A}, S(O)R^{b6A}, S(O)NR^{c6A}R^{d6A}, S(O)₂R^{b6A}, NR^{c6A}S(O)₂R^{b6A}, and S(O)₂NR^{c6A}R^{d6A};
R^{a6A}, R^{b6A}, R^{c6A}, and R^{d6A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a6A}, R^{b6A}, R^{c6A}, and R^{d6A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy; or
R^{c6A} and R^{d6A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;
R^{e6} and R^{e6A} are each, independently, H, CN or NO₂;
X⁷ is N or CR⁷;
R⁷ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, OR^{7A}, and NR^{7B}R^{7C}; wherein the C₃₋₁₀ cycloalkyl, 4-10 membered heterocycloalkyl, C₆₋₁₀ aryl, and 5-10 membered heteroaryl forming R⁷ are each optionally substituted with 1, 2, or 3 substituents independently selected from R^{7D}; wherein the C₁₋₃ alkyl, C₂₋₃ alkenyl, and C₂₋₃ alkynyl forming R⁷ are each optionally substituted with 1, 2, or 3 substituents independently selected from R^{7E};
R^{7A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{7A} are each optionally substituted with 1, 2, or 3 substituents independently selected from R^{7D}; wherein the C₁₋₃ alkyl, C₂₋₃ alkenyl, and C₂₋₃ alkynyl forming R^{7A} are each optionally substituted with 1, 2, or 3 substituents independently selected from R^{7E};
R^{7B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{7B} are each optionally substituted with 1, 2, or 3 substituents independently selected from R^{7D}; wherein the C₁₋₃ alkyl, C₂₋₃ alkenyl, and C₂₋₃ alkynyl forming R^{7B} are each optionally substituted with 1, 2, or 3 substituents independently selected from R^{7E};
R^{7C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{7C} are each optionally substituted with 1, 2, or 3 substituents independently selected from R^{7E}; or
R^{7B} and R^{7C}, together with the N atom to which they are both attached, optionally form a 4-, 5-, 6-, or 7-membered heterocycloalkyl group that is optionally substituted with 1, 2, or 3 substituents independently selected from independently selected from R^{7D};
each R^{7D} is independently selected from C₁₋₃ alkyl, C₁₋₃ haloalkyl, C₂₋₃ alkenyl, C₂₋₃ alkynyl, and R^{7E}; wherein each of the C₁₋₃ alkyl, C₂₋₃ alkenyl, and C₂₋₃ alkynyl forming R^{7D} is optionally substituted with 1, 2, or 3 substituents independently selected from R^{7E};
each R^{7E} is independently selected from D, halo, CN, OR^{a7}, SR^{a7}, C(O)R^{b7}, C(O)NR^{c7}R^{d7}, C(O)OR^{a7}, OC(O)R^{b7}, OC(O)NR^{c7}R^{d7}, NR^{c7}R^{d7}, NR^{c7}C(O)R^{b7}, NR^{c7}C(O)NR^{c7}R^{d7}, NR^{c7}C(O)OR^{a7}, C(═NR^{e7})NR^{c7}R^{d7}, NR^{c7}C(═NR^{e7})NR^{c7}R^{d7}, S(O)R^{b7}, S(O)NR^{c7}R^{d7}, S(O)₂R^{b7}, NR^{c7}S(O)₂R^{b7}, and S(O)₂NR^{c7}R^{d7};
R^{a7}, R^{b7}, R^{c7}, and R^{d7} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a7}, R^{b7}, R^{c7}, and R^{d7} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a7A}, SR^{a7A}, C(O)R^{b7A}, C(O)NR^{c7A}R^{d7A}, C(O)OR^{a7A}, OC(O)R^{b7A}, OC(O)NR^{c7A}R^{d7A}, NR^{c7A}R^{d7A}, NR^{c7A}C(O)R^{b7A}, NR^{c7A}C(O)NR^{c7A}R^{d7A}, NR^{c7A}C(O)OR^{a7A}, C(═NR^{e7A})NR^{c7A}R^{d7A}, NR^{c7A}C(═NR^{e7A})NR^{c7A}R^{d7A}, S(O)R^{b7A}, S(O)NR^{c7A}R^{d7A}, S(O)₂R^{b7A}, NR^{c7A}S(O)₂R^{b7A}, and S(O)₂NR^{c7A}R^{d7A}; or
R^{c7} and R^{d7} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a7A}, SR^{a7A}, C(O)R^{b7A}, C(O)NR^{c7A}R^{d7A}, C(O)OR^{a7A}, OC(O)R^{b7A}, OC(O)NR^{c7A}R^{d7A}, NR^{c7A}R^{d7A}, NR^{c7A}C(O)R^{b7A}, NR^{c7A}C(O)NR^{c7A}R^{d7A}, NR^{c7A}C(O)OR^{a7A}, C(═NR^{e7A})NR^{c7A}R^{d7A}, NR^{c7A}C(═NR^{e7A})NR^{c7A}R^{d7A}, S(O)R^{b7A}, S(O)NR^{c7A}R^{d7A}, S(O)₂R^{b7A}, NR^{c7A}S(O)₂R^{b7A}, and S(O)₂NR^{c7A}R^{d7A};
R^{a7A}, R^{b7A}, R^{c7A}, and R^{d7A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a7A}, R^{b7A}, R^{c7A}, and R^{d7A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy; or
R^{c7A} and R^{d7A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;
R^{e7} and R^{e7A} are each, independently, H, CN or NO₂;
X⁸ is N or CR⁸;
R⁸ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, CN, OR^{8A}, and NR^{8B}R^{8C}; wherein the C₃₋₁₀ cycloalkyl, 4-10 membered heterocycloalkyl, C₆₋₁₀ aryl, and 5-10 membered heteroaryl forming R⁸ are each optionally substituted with 1, 2, or 3 substituents independently selected from R^{8D}; wherein the C₁₋₃ alkyl, C₂₋₃ alkenyl, and C₂₋₃ alkynyl forming R⁸ are each optionally substituted with 1, 2, or 3 substituents independently selected from R^{8E};
R^{8A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{8A} are each optionally substituted with 1, 2, or 3 substituents independently selected from R^{8D}; wherein the C₁₋₃ alkyl, C₂₋₃ alkenyl, and C₂₋₃ alkynyl forming R^{8A} are each optionally substituted with 1, 2, or 3 substituents independently selected from R^{8E};
R^{8B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{8B} are each optionally substituted with 1, 2, or 3 substituents independently selected from R^{8D}; wherein the C₁₋₃ alkyl, C₂₋₃ alkenyl, and C₂₋₃ alkynyl forming R^{8B} are each optionally substituted with 1, 2, or 3 substituents independently selected from R^{8E};
R^{8C} is selected from H, C₁₋₃ alkyl, C₂₋₃ alkenyl, and C₂₋₃ alkynyl; wherein the C₁₋₃ alkyl, C₂₋₃ alkenyl, and C₂₋₃ alkynyl forming R^{8C} are each optionally substituted with 1, 2, or 3 substituents independently selected from R^{8E}; or
R^{8B} and R^{8C}, together with the N atom to which they are both attached, optionally form a 4-, 5-, 6-, or 7-membered heterocycloalkyl group that is optionally substituted with 1, 2, or 3 substituents independently selected from independently selected from R^{8D};
each R^{8D} is independently selected from C₁₋₃ alkyl, C₁₋₃ haloalkyl, C₂₋₃ alkenyl, C₂₋₃ alkynyl, and R^{8E}; wherein each of the C₁₋₃ alkyl, C₂₋₃ alkenyl, and C₂₋₃ alkynyl forming R^{8D} is optionally substituted with 1, 2, or 3 substituents independently selected from R^{8E};
each R^{8E} is independently selected from D, halo, CN, OR^{a8}, SR^{a8}, C(O)R^{b8}, C(O)NR^{c8}R^{d8}, C(O)OR^{a8}, OC(O)R^{b8}, OC(O)NR^{c8}R^{d8}, NR^{c8}R^{d8}, NR^{c8}C(O)R^{b8}, NR^{c8}C(O)NR^{c8}R^{d8}, NR^{c8}C(O)OR^{a8}, C(═NR^{e8})NR^{c8}R^{d8}, NR^{c8}C(═NR^{e8})NR^{c8}R^{d8}, S(O)R^{b8}, S(O)NR^{c8}R^{d8}, S(O)₂R^{b8}, NR^{c8}S(O)₂R^{b8}, and S(O)₂NR^{c8}R^{d8};
R^{a8}, R^{b8}, R^{c8}, and R^{d8} are each independently selected from H, C₁₋₃ alkyl, C₂₋₃ alkenyl, C₂₋₃ alkynyl, C₆₋₁₀ aryl, C₃₋₇ cycloalkyl, 5-10 membered heteroaryl, 4-10 membered heterocycloalkyl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a8}, R^{b8}, R^{c8}, and R^{d8} are each optionally substituted with 1, 2, 3, 4, or 5 substituents independently selected from C₁₋₃ alkyl, C₁₋₃ haloalkyl, C₂₋₃ alkenyl, C₂₋₃ alkynyl, halo, CN, OR^{a8A}, SR^{a8A}, C(O)R^{b8A}, C(O)NR^{c8A}R^{d8A}, C(O)OR^{a8A}, OC(O)R^{b8A}, OC(O)NR^{c8A}R^{d8A}, NR^{c8A}R^{d8A}, NR^{c8A}C(O)R^{b8A}, NR^{c8A}C(O)NR^{c8A}R^{d8A}, NR^{c8A}C(O)OR^{a8A}, C(═NR^{e8A})NR^{c8A}R^{d8A}, NR^{c8A}C(═NR^{e8A})NR^{c8A}R^{d8A}, S(O)R^{b8A}, S(O)NR^{c8A}R^{d8A}, S(O)₂R^{b8A}, NR^{c8A}S(O)₂R^{b8A}, and S(O)₂NR^{c8A}R^{d8A}; or
R^{c8} and R^{d8} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from C₁₋₃ alkyl, C₁₋₃ haloalkyl, C₂₋₃ alkenyl, C₂₋₃ alkynyl, halo, CN, OR^{a8A}, SR^{a8A}, C(O)R^{b8A}, C(O)NR^{c8A}R^{d8A}, C(O)OR^{a8A}, OC(O)R^{b8A}, OC(O)NR^{c8A}R^{d8A}, NR^{c8A}R^{d8A}, NR^{c8A}C(O)R^{b8A}, NR^{c8A}C(O)NR^{c8A}R^{d8A}, NR^{c8A}C(O)OR^{a8A}, C(═NR^{e8A})NR^{c8A}R^{d8A}, NR^{c8A}C(═NR^{e8A})NR^{c8A}R^{d8A}, S(O)R^{b8A}, S(O)NR^{c8A}R^{d8A}, S(O)₂R^{b8A}, NR^{c8A}S(O)₂R^{b8A}, and S(O)₂NR^{c8A}R^{d8A};
R^{a8A}, R^{b8A}, R^{c8A}, and R^{d8A} are each independently selected from H, C₁₋₃ alkyl, C₁₋₃ haloalkyl, C₂₋₃ alkenyl, C₂₋₃ alkynyl, aryl, C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl; wherein the C₁₋₆ alkyl, C₁₋₆ haloalkyl, C₂₋₆ alkenyl, C₂₋₆ alkynyl C₆₋₁₀ aryl-C₁₋₃ alkyl, 5-10 membered heteroaryl-C₁₋₃ alkyl, C₃₋₇ cycloalkyl-C₁₋₃ alkyl, and 4-10 membered heterocycloalkyl-C₁₋₃ alkyl forming R^{a8A}, R^{b8A}, R^{c8A}, and R^{d8A} are each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy; or
R^{c8A} and R^{d8A} attached to the same N atom, together with the N atom to which they are both attached, form a 4-, 5-, 6-, or 7-membered heterocycloalkyl group or 5-membered heteroaryl group, each optionally substituted with 1, 2, or 3 substituents independently selected from OH, CN, amino, NH(C₁₋₆ alkyl), N(C₁₋₆ alkyl)₂, halo, C₁₋₆ alkyl, C₁₋₆ alkoxy, C₁₋₆ haloalkyl, and C₁₋₆ haloalkoxy;
R^{e8} and R^{e8A} are each, independently, H, CN or NO₂;
X⁹ is N or CR⁹;
R⁹ is selected from H, C₁₋₃ alkyl, C₁₋₃ haloalkyl, C₂₋₃ alkenyl, C₂₋₃ alkynyl, CN, halo, OR^{9A}, and NR^{9B}R^{9C};
R^{9A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{9B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{9B} are each optionally substituted with 1, 2, or 3 substituents independently selected from R^{9D};
R^{9C} is selected from H, C₁₋₃ alkyl, C₂₋₃ alkenyl, and C₂₋₃ alkynyl;
each R^{9D} is independently selected from C₁₋₃ alkyl, C₁₋₃ haloalkyl, C₂₋₃ alkenyl, and C₂₋₃ alkynyl;
R¹⁰ is selected from H, C₁₋₃ alkyl, C₁₋₃ haloalkyl, C₂₋₃ alkenyl, C₂₋₃ alkynyl, CN, halo, OR^{10A}, and NR^{10B}R^{10C};
R^{10A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{10B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{10B} are each optionally substituted with 1, 2, or 3 substituents independently selected from R^{10D};
R^{10C} is selected from H, C₁₋₃ alkyl, C₂₋₃ alkenyl, and C₂₋₃ alkynyl;
each R^{10D} is independently selected from C₁₋₃ alkyl, C₁₋₃ haloalkyl, C₂₋₃ alkenyl, and C₂₋₃ alkynyl;
R¹¹ is selected from H, C₁₋₃ alkyl, C₁₋₃ haloalkyl, C₂₋₃ alkenyl, C₂₋₃ alkynyl, CN, halo, OR^{11A}, and NR^{11B}R^{11C};
R^{11A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{11B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{11B} are each optionally substituted with 1, 2, or 3 substituents independently selected from R^{11D};
R^{11C} is selected from H, C₁₋₃ alkyl, C₂₋₃ alkenyl, and C₂₋₃ alkynyl;
each R^{11D} is independently selected from C₁₋₃ alkyl, C₁₋₃ haloalkyl, C₂₋₃ alkenyl, and C₂₋₃ alkynyl;
X¹² is O or S;
R¹³ is independently selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{13A}, and NR^{13B}R^{13C};
R^{13A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{13B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{13B} are each optionally substituted with 1, 2, or 3 substituents independently selected from R^{13D};
R^{13C} is selected from H, C₁₋₃ alkyl, C₂₋₃ alkenyl, and C₂₋₃ alkynyl;
each R^{13D} is independently selected from C₁₋₃ alkyl, C₁₋₃ haloalkyl, C₂₋₃ alkenyl, and C₂₋₃ alkynyl;
R¹⁴ is independently selected from C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{14A}, and NR^{14B}R^{14C};
R^{14A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{14B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{14B} are each optionally substituted with 1, 2, or 3 substituents independently selected from R^{14D};
R^{14C} is selected from H, C₁₋₃ alkyl, C₂₋₃ alkenyl, and C₂₋₃ alkynyl; and
each R^{14D} is independently selected from C₁₋₃ alkyl, C₁₋₃ haloalkyl, C₂₋₃ alkenyl, and C₂₋₃ alkynyl;
wherein the ring-forming atoms of heterocycloalkyl and heteroaryl consist of at least one ring-forming carbon atom and 1, 2, or 3 heteroatoms independently selected from N, O, and S; and wherein a ring-forming carbon atom of the heterocycloalkyl and heteroaryl is optionally substituted by oxo to form a carbonyl group.

2. The compound of claim 1, wherein the compound of Formula (I) is a compound of Formula (Ia), (Ib), (Ic), (Id), (Ie), or (If): 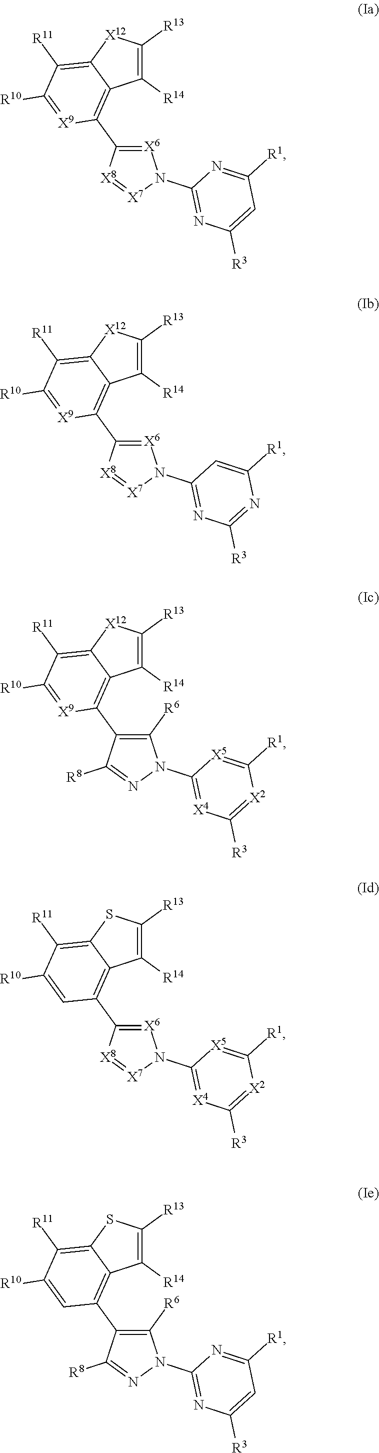 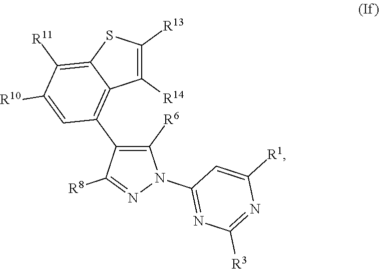
or a pharmaceutically acceptable salt thereof.

3. The compound of claim 1, wherein the compound of Formula (I) is a compound of Formula (Ig), (Ih), (Ii), (Ij), (Ik), (Im), or (In): 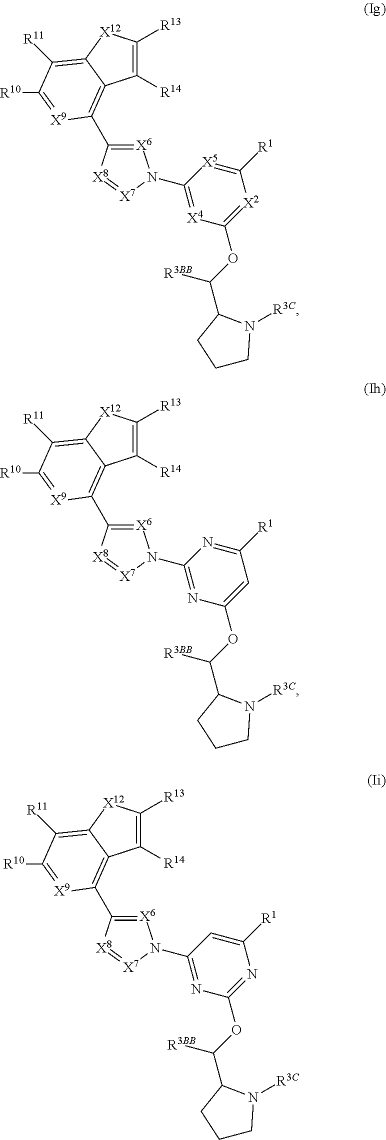 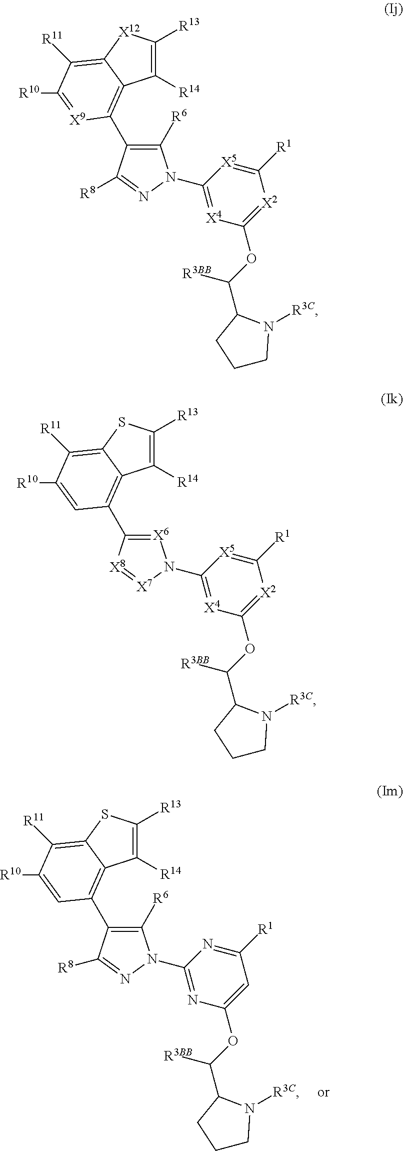 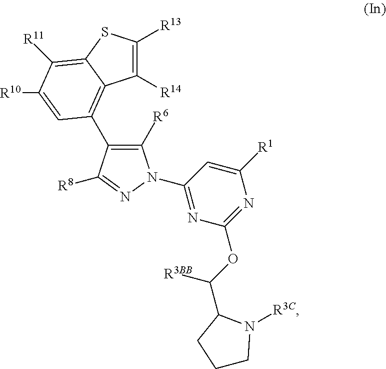
or a pharmaceutically acceptable salt thereof,
wherein R^{3BB} is H or C₁₋₃ alkyl.

4. The compound of claim 1, or a pharmaceutically acceptable salt thereof, wherein
R¹ is selected from H, D, C₁₋₃ alkyl, C₁₋₃ haloalkyl, C₂₋₃ alkenyl, C₂₋₃ alkynyl, C₃₋₅ cycloalkyl, 4-10 membered heterocycloalkyl, phenyl, 5-10 membered heteroaryl, halo, CN, OR^{a1}, C(O)R^{b1}, NR^{c1B}C(O)R^{b1B}, C(O)NR^{c1}R^{d1}, NR^{c1}R^{e1}, and NR^{c1}C(O)R^{b1}; wherein the C₃₋₅cycloalkyl, 4-10 membered heterocycloalkyl, phenyl, and 5-10 membered heteroaryl forming R¹ are each optionally substituted with 1, 2, or 3 substituents independently selected from R^{1A};
each R^{1A} is independently selected from C₁₋₃ alkyl, C₁₋₃ haloalkyl, and R^{1B}; wherein the C₁₋₃ alkyl forming R^{1A} is optionally substituted with 1, 2, or 3 substituents each independently selected from R^{1B};
each R^{1B} is independently selected from D, C₃₋₆cycloalkyl, 4-10 membered heterocycloalkyl, phenyl, 5-6 membered heteroaryl, halo, CN, OR^{a1B}, C(O)R^{b1B}, C(O)NR^{c1B}R^{d1B}, C(O)OR^{a1B}, and NR^{c1B}R^{d1B}; wherein the C₃₋₆cycloalkyl, 4-6 membered heterocycloalkyl, phenyl, and 5-6 membered heteroaryl forming R^{1B} are each optionally substituted with 1, 2, or 3 substituents independently selected from R^{1C};
each R^{1C} is independently selected from D, C₃₋₆cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C}; wherein the C₃₋₆cycloalkyl, 4-6 membered heterocycloalkyl, phenyl, and 5-6 membered heteroaryl forming R^{1C} are each optionally substituted with 1, 2, or 3 substituents independently selected from R^{1D};
each R^{1D} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a1D}, C(O)R^{b1D}, C(O)NR^{c1D}R^{d1D}, C(O)OR^{a1D}, NR^{c1D}R^{d1D}, and S(O)₂R^{b1D};
each R^{a1} is independently selected from C₃₋₆ cycloalkyl, 4-6 membered heterocycloalkyl, phenyl, and 5-6 membered heteroaryl; wherein the C₃₋₆cycloalkyl, 4-6 membered heterocycloalkyl, phenyl, and 5-6 membered heteroaryl forming R^{a1} are each optionally substituted with 1, 2, or 3 substituents independently selected from R^{1A};
each R^{b1}, R^{c1}, and R^{d1} is independently selected from H, C₁₋₃ alkyl, C₁₋₃ haloalkyl, C₂₋₃ alkenyl, and C₂₋₃ alkynyl;
each R^{e1} is independently selected from C₁₋₃ alkyl, C₁₋₃ haloalkyl, C₂₋₃ alkenyl, and C₂₋₃ alkynyl; wherein the C₁₋₃ alkyl, C₁₋₃ haloalkyl, C₂₋₃ alkenyl, and C₂₋₃ alkynyl forming R^{e1} are each optionally substituted with 1, 2, or 3 substituents independently selected from R^{1B};
each R^{a1B}, R^{b1B}, R^{c1B}, and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1C}, R^{b1C}, R^{c1C}, and R^{d1C} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1D}, R^{b1D}, R^{c1D}, and R^{d1D} is independently selected from H, C₁₋₃ alkyl, and C₁₋₃ haloalkyl;
X² is N or CH;
R³ is selected from H, D, C₁₋₃ alkyl, C₁₋₃ haloalkyl, C₂₋₃ alkenyl, C₂₋₃ alkynyl, C₃₋₅ cycloalkyl, 4-9 membered heterocycloalkyl, phenyl, 5-6 membered heteroaryl, C₃₋₆ cycloalkyl-C₁₋₃ alkylene, 4-6 membered heterocycloalkyl-C₁₋₃ alkylene, phenyl-C₁₋₃ alkylene, 5-6 membered heteroaryl-C₁₋₃ alkylene, halo, CN, OR^{a3}, C(O)R^{b3}, C(O)NR^{c3}R^{d3}, NR^{c3}R^{e3}, and NR^{c3}C(O)R^{b3}, wherein 4-9 membered heterocycloalkyl is optionally substituted with R^{3A};
each R^{3A} is independently selected from C₁₋₃ alkyl, C₁₋₃ haloalkyl, C₂₋₃ alkenyl, C₂₋₃ alkynyl, and R^{3B};
each R^{3B} is independently selected from D, C₃₋₆ cycloalkyl, 4-10 membered heterocycloalkyl, phenyl, 5-6 membered heteroaryl, halo, CN, and NR^{c3B}C(O)R^{b3B}; wherein the C₃₋₆cycloalkyl, 4-6 membered heterocycloalkyl, phenyl, and 5-6 membered heteroaryl forming R^{3B} are each optionally substituted with 1, 2, or 3 substituents independently selected from R^{3C};
each R^{3C} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, and CN;
each R^{a3} is independently selected from C₁₋₆ alkyl, C₁₋₃ haloalkyl, C₂₋₆ alkenyl, C₂₋₆ alkynyl, C₃₋₆ cycloalkyl, 4-6 membered heterocycloalkyl, phenyl, and 5-6 membered heteroaryl; wherein the C₁₋₆ alkyl, C₂₋₆ alkenyl, and C₂₋₆ alkynyl forming R^{a3} are each optionally substituted with 1, 2, or 3 substituents independently selected from R^{3B};
X⁴ is N;
X⁵ is N or CR⁵;
R⁵ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, halo, CN, OR^{5A}, and NR^{5B}R^{5C};
R^{5A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{5B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, and 5-10 membered heteroaryl;
R^{5C} is selected from H, C₁₋₃ alkyl, C₂₋₃ alkenyl, and C₂₋₃ alkynyl;
X⁶ is CR⁶;
R⁶ is selected from C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, halo, CN, OR^{6A}, and NR^{6B}R^{6C};
R^{6A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{6B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{6C} is selected from H, C₁₋₃ alkyl, C₂₋₃ alkenyl, and C₂₋₃ alkynyl;
X⁷ is N, CH, or C-halo;
X⁸ is CR⁸;
R⁸ is selected from H, C₁₋₃ alkyl, C₁₋₃ haloalkyl, C₂₋₃ alkenyl, C₂₋₃ alkynyl, C₃₋₁₀cycloalkyl, 4-10 membered heterocycloalkyl, C₆₋₁₀ aryl, 5-10 membered heteroaryl, halo, CN, OR^{8A}, and NR^{8B}R^{8C};
R^{8A} is selected from C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{8B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{8C} is selected from H, C₁₋₃ alkyl, C₂₋₃ alkenyl, and C₂₋₃ alkynyl;
X⁹ is N or CH;
R¹⁰ is selected from H, C₁₋₃ alkyl, C₁₋₃ haloalkyl, C₂₋₃ alkenyl, C₂₋₃ alkynyl, CN, halo, OR^{10A}, and NR^{10B}R^{10C};
R^{10A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{10B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{10B} are each optionally substituted with 1, 2, or 3 substituents independently selected from R^{10D};
R^{10C} is selected from H, C₁₋₃ alkyl, C₂₋₃ alkenyl, and C₂₋₃ alkynyl;
each R^{10D} is independently selected from C₁₋₃ alkyl, C₁₋₃ haloalkyl, C₂₋₃ alkenyl, and C₂₋₃ alkynyl;
R¹¹ is selected from H, C₁₋₃ alkyl, C₁₋₃ haloalkyl, C₂₋₃ alkenyl, C₂₋₃ alkynyl, CN, halo, OR^{11A}, and NR^{11B}R^{11C};
R^{11A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{11B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; wherein the C₃₋₁₀ cycloalkyl, 4-10 membered heterocycloalkyl, C₆₋₁₀ aryl, and 5-10 membered heteroaryl forming R^{11B} are each optionally substituted with 1, 2, or 3 substituents independently selected from R^{11D};
R^{11C} is selected from H, C₁₋₃ alkyl, C₂₋₃ alkenyl, and C₂₋₃ alkynyl;
each R^{11D} is independently selected from C₁₋₃ alkyl, C₁₋₃ haloalkyl, C₂₋₃ alkenyl, and C₂₋₃ alkynyl;
X¹² is O or S;
R¹³ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{13A}, and NR^{13B}R^{13C};
R^{13A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{13B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{13C} is selected from H, C₁₋₃ alkyl, C₂₋₃ alkenyl, and C₂₋₃ alkynyl;
R¹⁴ is selected from H, C₁₋₃ alkyl, C₁₋₃ haloalkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl, CN, halo, OR^{14A}, and NR^{14B}R^{14C};
R^{14A} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl;
R^{14B} is selected from H, C₁₋₃ alkyl, C₂₋₃ alkenyl, C₂₋₃ alkynyl, C₃₋₁₀ cycloalkyl, 4-10 membered heterocycloalkyl, C₆₋₁₀ aryl, 5-10 membered heteroaryl; and
R^{14C} is selected from H, C₁₋₃ alkyl, C₂₋₃ alkenyl, and C₂₋₃ alkynyl.

5. The compound of claim 1, or a pharmaceutically acceptable salt thereof, wherein
R¹ is selected from C₃₋₅cycloalkyl, 4-10 membered heterocycloalkyl, phenyl, 5-10 membered heteroaryl, halo, CN, OR^{a1}, C(O)R^{a1}, C(O)NR^{b1}, C(O)NR^{c1}R^{d1}, NR^{c1}R^{e1}, and NR^{c1}C(O)R^{b1}; wherein the C₃₋₅cycloalkyl, 4-10 membered heterocycloalkyl, phenyl, and 5-10 membered heteroaryl forming R¹ are each optionally substituted with 1 or 2 substituents independently selected from R^{1A};
each R^{1A} is independently selected from C₁₋₃ alkyl, C₁₋₃ haloalkyl, and R^{1B}; wherein the C₁₋₃ alkyl forming R^{1A} is optionally substituted with 1 or 2 substituents each independently selected from R^{1B};
each R^{1B} is independently selected from C₃₋₆ cycloalkyl, 4-10 membered heterocycloalkyl, phenyl, 5-6 membered heteroaryl, halo, CN, OR^{a1B}, C(O)R^{b1B}, NR^{c1B}C(O)R^{b1B}, C(O)NR^{c1B}R^{d1B}, C(O)OR^{a1B}, and NR^{c1B}R^{d1B}; wherein the C₃₋₆ cycloalkyl, 4-6 membered heterocycloalkyl, phenyl, and 5-6 membered heteroaryl forming R^{1B} are each optionally substituted with 1 or 2 substituents independently selected from R^{1C};
each R^{1C} is independently selected from D, C₃₋₆ cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C}; wherein the C₃₋₆-cycloalkyl, 4-6 membered heterocycloalkyl, phenyl, and 5-6 membered heteroaryl forming R^{1C} are each optionally substituted with 1, 2, or 3 substituents independently selected from R^{1D};
each R^{1D} is independently selected from C₁₋₃ alkyl, C₁₋₃ haloalkyl, C₂₋₃ alkenyl, C₂₋₃ alkynyl, C₃₋₆ cycloalkyl, 4-6 membered heterocycloalkyl, phenyl, 5-6 membered heteroaryl, halo, D, CN, OR^{a1D}, C(O)R^{b1D}, C(O)NR^{c1D}R^{d1D}, C(O)OR^{a1D}, NR^{c1D}R^{d1D}, and S(O)₂R^{b1D};
each R^{a1} is independently selected from C₃₋₆-cycloalkyl, 4-6 membered heterocycloalkyl, phenyl, and 5-6 membered heteroaryl; wherein the C₃₋₆-cycloalkyl, 4-6 membered heterocycloalkyl, phenyl, and 5-6 membered heteroaryl forming R^{a1} are each optionally substituted with 1 or 2 substituents independently selected from R^{1A};
each R^{b1}, R^{c1}, and R^{d1} is independently selected from H and C₁₋₃ alkyl;
each R^{e1} is independently selected from C₁₋₃ alkyl; wherein the C₁₋₃ alkyl forming R^{e1} is optionally substituted with 1 or 2 substituents independently selected from R^{1B};
each R^{a1B}, R^{b1B}, R^{c1B}, and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1C}, R^{b1C}, R^{c1C}, and R^{d1C} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl;
each R^{a1D}, R^{b1D}, R^{c1D}, and R^{d1D} is independently selected from H, C₁₋₃ alkyl, and C₁₋₃ haloalkyl;
X² is N or CH;
R³ is selected from C₃₋₅ cycloalkyl, 4-9 membered heterocycloalkyl, phenyl, 5-6 membered heteroaryl, halo, CN, and OR^{a3}, wherein 4-9 membered heterocycloalkyl is optionally substituted with R^{3A};
each R^{3A} is independently selected from C₁₋₃ alkyl, C₁₋₃ haloalkyl, C₂₋₃ alkenyl, C₂₋₃ alkynyl, and R^{3B};
each R^{3B} is independently selected from C₃₋₆ cycloalkyl, 4-10 membered heterocycloalkyl, phenyl, 5-6 membered heteroaryl, and NR^{c3B}C(O)R^{b3B}; wherein the C₃₋₆cycloalkyl, 4-6 membered heterocycloalkyl, phenyl, and 5-6 membered heteroaryl forming R^{3B} are each optionally substituted with 1 or 2 substituents independently selected from R^{3C};
each R^{3C} is independently selected from C₁₋₃ alkyl, C₁₋₃ haloalkyl, halo, D, and CN;
each R^{a3} is independently selected from C₁₋₆ alkyl and C₁₋₃ haloalkyl; wherein the C₁₋₆ alkyl forming R^{a3} are each optionally substituted with 1 or 2 substituents independently selected from R^{3B};
X⁴ is N;
X⁵ is N or CR⁵;
R⁵ is selected from H, C₁₋₃ alkyl, C₁₋₃ haloalkyl, C₂₋₃ alkenyl, C₂₋₃ alkynyl, halo, CN, OR^{5A}, and NR^{5B}R^{5C};
R^{5A} is selected from C₁₋₃ alkyl, C₂₋₃ alkenyl, and C₂₋₃ alkynyl;
R^{5B} is selected from H and C₁₋₃ alkyl;
R^{5C} is selected from H and C₁₋₃ alkyl;
X⁶ is CR⁶;
R⁶ is selected from C₁₋₃ alkyl, C₁₋₃ haloalkyl, C₂₋₃ alkenyl, C₂₋₃ alkynyl, halo, and CN;
X⁷ is N or CH;
X⁸ is CR⁸;
R⁸ is selected from H, C₁₋₃ alkyl, C₁₋₃ haloalkyl, C₂₋₃ alkenyl, C₂₋₃ alkynyl, halo, and CN;
X⁹ is N or CH;
R¹⁰ is selected from H, C₁₋₃ alkyl, C₁₋₃ haloalkyl, CN, and halo;
R¹¹ is selected from H, C₁₋₃ alkyl, C₁₋₃ haloalkyl, CN, and halo;
X¹² is O or S;
R¹³ is selected from H, C₁₋₃ alkyl, C₁₋₃ haloalkyl, C₂₋₃ alkenyl, C₂₋₃ alkynyl, CN, halo, OR^{13A}, and NR^{13B}R^{13C};
R^{13A} is selected from H and C₁₋₃ alkyl;
R^{13B} is selected from H and C₁₋₃ alkyl;
R^{13C} is selected from H and C₁₋₃ alkyl;
R¹⁴ is selected from H, C₁₋₃ alkyl, C₁₋₃ haloalkyl, C₂₋₃ alkenyl, C₂₋₃ alkynyl, CN, halo, OR^{14A}, and NR^{14B}R^{14C};
R^{14A} is selected from H and C₁₋₃ alkyl;
R^{14B} is selected from H and C₁₋₃ alkyl; and
R^{14C} is selected from H and C₁₋₃ alkyl.

6. The compound of claim 1, or a pharmaceutically acceptable salt thereof, wherein
R¹ is selected from C₃₋₅cycloalkyl, 4-10 membered heterocycloalkyl, phenyl, 5-10 membered heteroaryl, halo, OR^{a1}, and NR^{c1}R^{e1}; wherein the C₃₋₅cycloalkyl, 4-10 membered heterocycloalkyl, phenyl, and 5-10 membered heteroaryl forming R¹ are each optionally substituted with 1 or 2 substituents independently selected from R^{1A};
each R^{1A} is independently selected from C₁₋₃ alkyl, C₁₋₃ haloalkyl, and R^{1B}; wherein the C₁₋₃ alkyl forming R^{1A} is optionally substituted with 1 or 2 substituents each independently selected from R^{1B};
each R^{1B} is independently selected from C₃₋₆cycloalkyl, 4-10 membered heterocycloalkyl, phenyl, 5-6 membered heteroaryl, halo, OR^{a1B}, C(O)R^{b1B}, NR^{c1B}C(O)R^{b1B}, and C(O)NR^{c1B}R^{d1B}; wherein the C₃₋₆cycloalkyl, 4-6 membered heterocycloalkyl, phenyl, and 5-6 membered heteroaryl forming R^{1B} are each optionally substituted with 1 or 2 substituents independently selected from R^{1C};
each R^{1C} is independently selected from D, C₃₋₆cycloalkyl, 4-10 membered heterocycloalkyl, phenyl, 5-6 membered heteroaryl, halo, CN, OR^{a1C}, C(O)R^{b1C}, C(O)NR^{c1C}R^{d1C}, C(O)OR^{a1C}, NR^{c1C}R^{d1C}, and S(O)₂R^{b1C};
each R^{a1} is independently selected from C₃₋₆cycloalkyl, 4-6 membered heterocycloalkyl, phenyl, and 5-6 membered heteroaryl; wherein the C₃₋₆cycloalkyl, 4-6 membered heterocycloalkyl, phenyl, and 5-6 membered heteroaryl forming R^{a1} are each optionally substituted with 1 or 2 substituents independently selected from R^{1A};
R^{c1} is selected from H and C₁₋₃ alkyl;
R^{e1} is selected from C₁₋₃ alkyl; wherein the C₁₋₃ alkyl forming R^{e1} is optionally substituted with 1 or 2 substituents independently selected from R^{1B};
each R^{a1B}, R^{b1B}, R^{c1B}, and R^{d1B} is independently selected from H, C₁₋₃ alkyl, C₁₋₃ haloalkyl, C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene, wherein the C₃₋₆ cycloalkyl, 4-6 membered heterocycloalkyl, C₃₋₆ cycloalkyl-C₁₋₃ alkylene, 4-10 membered heterocycloalkyl-C₁₋₃ alkylene, phenyl-C₁₋₃ alkylene, and 5-10 membered heteroaryl-C₁₋₃ alkylene are each optionally substituted with 1, 2, or 3 substituents selected from D, halo, C₁₋₃ alkyl, C₁₋₃ haloalkyl, CN, C(O)C₁₋₃ alkyl, and C(O)OC₁₋₃ alkyl; and
each R^{a1C}, R^{b1C}, R^{c1C}, and R^{d1C} is independently selected from H and C₁₋₃ alkyl.

7-8. (canceled)

9. The compound of claim 1, or a pharmaceutically acceptable salt thereof, wherein
R³ is selected from C₃₋₅ cycloalkyl, 4-9 membered heterocycloalkyl, halo, and OR^{a3}, wherein 4-9 membered heterocycloalkyl is optionally substituted with R^{3A};
each R^{3A} is independently selected from C₁₋₃ alkyl, C₁₋₃ haloalkyl, C₂₋₃ alkenyl, C₂₋₃ alkynyl, and R^{3B};
each R^{3B} is independently selected from C₃₋₆ cycloalkyl, 4-10 membered heterocycloalkyl, and NR^{c3B}C(O)R^{b3B}; wherein the C₃₋₆cycloalkyl and 4-6 membered heterocycloalkyl forming R^{3B} are each optionally substituted with 1 or 2 substituents independently selected from R^{3C};
each R^{3C} is independently selected from C₁₋₃ alkyl, C₁₋₃ haloalkyl, and halo; and
each R^{a3} is independently selected from C₁₋₆ alkyl and C₁₋₃ haloalkyl; wherein the C₁₋₆ alkyl forming R^{a3} are each optionally substituted with 1 or 2 substituents independently selected from R^{3B}.

10-11. (canceled)

12. The compound of claim 1, or a pharmaceutically acceptable salt thereof, wherein X⁵ is CR⁵; and
R⁵ is selected from H, C₁₋₃ alkyl, C₁₋₃ haloalkyl, halo, and CN.

13. (canceled)

14. The compound of claim 1, or a pharmaceutically acceptable salt thereof, wherein
X⁶ is CR⁶; and
R⁶ is selected from C₁₋₃ alkyl, C₁₋₃ haloalkyl, halo, and CN.

15. (canceled)

16. The compound of claim 1, or a pharmaceutically acceptable salt thereof, wherein
X⁸ is CR⁸; and
R³ is selected from C₁₋₃ alkyl, C₁₋₃ haloalkyl, halo, and CN.

17. (canceled)

18. The compound of claim 1, or a pharmaceutically acceptable salt thereof, wherein R¹⁰ is H or halo; and R¹¹ is H or halo.

19-20. (canceled)

21. The compound of claim 1, or a pharmaceutically acceptable salt thereof, wherein R¹³ is selected from H, C₁₋₃ alkyl, C₁₋₃ haloalkyl, CN, halo, OH, and NH₂; and
R¹⁴ is selected from H, C₁₋₃ alkyl, C₁₋₃ haloalkyl, CN, halo, OH, and NH₂.

22. (canceled)

23. A compound selected from
4-(1-(4-(4-acetylpiperazin-1-yl)-6-(1-(1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
1-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N-methylpyrrolidine-3-carboxamide;
2-amino-4-(3,5-dimethyl-1-(4-(4-methyl-3-oxopiperazin-1-yl)-6-(1-(1-methylpyrrolidin-2-yl)ethoxy)pyrimidin-2-yl)-1H-pyrazol-4-yl)-7-fluorobenzo[b]thiophene-3-carbonitrile;
2-amino-7-fluoro-4-(1-(4-(4-hydroxy-4-methylpiperidin-1-yl)-6-(1-(1-methylpyrrolidin-2-yl)ethoxy)pyrimidin-2-yl)-3,5-dimethyl-1H-pyrazol-4-yl)benzo[b]thiophene-3-carbonitrile;
4-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N,N-dimethylpiperazine-1-carboxamide;
4-(1-(4-(6-acetyl-2,6-diazaspiro[3.3]heptan-2-yl)-6-(1-(1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(1-(4-(4-(cyclopropanecarbonyl)piperazin-1-yl)-6-(1-(1-methylpyrrolidin-2-yl)ethoxy)pyrimidin-2-yl)-3,5-dimethyl-1H-pyrazol-4-yl)-7-fluorobenzo[b]thiophene-3-carbonitrile;
methyl 4-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)piperazine-1-carboxylate;
2-amino-4-(3,5-dimethyl-1-(4-(2-methyl-3-oxohexahydroimidazo[1,5-a]pyrazin-7(1H)-yl)-6-(1-(1-methylpyrrolidin-2-yl)ethoxy)pyrimidin-2-yl)-1H-pyrazol-4-yl)-7-fluorobenzo[b]thiophene-3-carbonitrile;
4-(1-(4-(6-acetyl-2,6-diazaspiro[3.4]octan-2-yl)-6-(1-(1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(3-acetyl-3,8-diazabicyclo[3.2.1]octan-8-yl)-6-(1-(1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(4-acetyl-3-(cyanomethyl)piperazin-1-yl)-6-(1-(1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(1-(4-(5,6-dihydroimidazo[1,5-a]pyrazin-7(8H)-yl)-6-(1-(1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[4,3-a]pyrazin-7(8H)-yl)-6-(1-(1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(3,5-dimethyl-1-(4-(1-(1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-4-(3,5-dimethyl-1-(4-(1-(1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-4-(1-(4-(5,6-dihydro-[1,2,4]triazolo[1,5-a]pyrazin-7(8H)-yl)-6-(1-(1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(3,5-dimethyl-1-(4-(1-(1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-4-(3,5-dimethyl-1-(4-(1-(1-methylpyrrolidin-2-yl)ethoxy)-6-((1-(pyridin-3-yl)ethyl)amino)pyrimidin-2-yl)-1H-pyrazol-4-yl)-7-fluorobenzo[b]thiophene-3-carbonitrile;
4-(1-(4-((1-acetylpiperidin-4-yl)amino)-6-(1-(1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(3,5-dimethyl-1-(4-(1-methyl-1H-pyrazol-4-yl)-6-(1-(1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-3-yl)-6-(1-(1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(3-methylisoxazol-5-yl)-6-(1-(1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-6-oxo-1,6-dihydropyridin-3-yl)-6-(1-(1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-(1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-7-fluoro-4-(1-(4-(6-(2-hydroxypropan-2-yl)pyridin-3-yl)-6-(1-(1-methylpyrrolidin-2-yl)ethoxy)pyrimidin-2-yl)-3,5-dimethyl-1H-pyrazol-4-yl)benzo[b]thiophene-3-carbonitrile;
5-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N,N-dimethylpicolinamide;
5-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N-methylpicolinamide;
2-amino-4-(3,5-dimethyl-1-(6-(1-(1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-4-(3,5-dimethyl-1-(4-(1-(1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
1-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-1H-pyrazole-3-carboxamide;
1-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N-methyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N,N-dimethyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N-methyl-1H-pyrazole-4-carboxamide;
2-amino-4-(3,5-dimethyl-1-(4-(1-(1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
4-(1-(4-((1-acetylpiperidin-4-yl)oxy)-6-(1-(1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(3,5-dimethyl-1-(6-(1-methyl-1H-pyrazol-4-yl)-2-(1-(1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(2-(1-(1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-4-(1-(6-(1-(difluoromethyl)-1H-pyrazol-4-yl)-2-(1-(1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(3,5-dimethyl-1-(2-(1-(1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-4-(1-(6-(1-benzyl-1H-pyrazol-4-yl)-2-(1-(1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(3,5-dimethyl-1-(2-(1-(1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-7-fluoro-4-(1-(6-(5-fluoro-1-methyl-1H-pyrazol-4-yl)-2-(1-(1-methylpyrrolidin-2-yl)ethoxy)pyrimidin-4-yl)-3,5-dimethyl-1H-pyrazol-4-yl)benzo[b]thiophene-3-carbonitrile;
2-amino-4-(1-(6-(1,3-dimethyl-1H-pyrazol-4-yl)-2-(1-(1-methylpyrrolidin-2-yl)ethoxy)pyrimidin-4-yl)-3,5-dimethyl-1H-pyrazol-4-yl)-7-fluorobenzo[b]thiophene-3-carbonitrile;
4-(6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidin-4-yl)-N-methylpicolinamide;
2-amino-4-(1-(6-(1-(cyanomethyl)-1H-pyrazol-4-yl)-2-(1-(1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(4-(cyclopropanecarbonyl)piperazin-1-yl)-2-(1-(1-methylpyrrolidin-2-yl)ethoxy)pyrimidin-4-yl)-3,5-dimethyl-1H-pyrazol-4-yl)-7-fluorobenzo[b]thiophene-3-carbonitrile;
4-(1-(6-(4-acetylpiperazin-1-yl)-2-(1-(1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
5-(6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
1-(6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidin-4-yl)-1H-pyrazole-3-carboxamide;
4-(1-(6-(4-acetylpiperazin-1-yl)-5-fluoro-2-(1-(1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(4-acetylpiperazin-1-yl)-6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidine-5-carbonitrile;
2-amino-4-(1-(4-(4-(cyclopropanecarbonyl)piperazin-1-yl)-6-(1-(1-methylpyrrolidin-2-yl)ethoxy)pyrimidin-2-yl)-3-methyl-1H-pyrazol-4-yl)-7-fluorobenzo[b]thiophene-3-carbonitrile;
2-amino-7-fluoro-4-(1-(2-((2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
1-(2-(4-(2-amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide;
2-amino-4-(1-(2-(4-(cyclopropanecarbonyl)piperazin-1-yl)-6-(1-(1-methylpyrrolidin-2-yl)ethoxy)pyrimidin-4-yl)-3,5-dimethyl-1H-pyrazol-4-yl)-7-fluorobenzo[b]thiophene-3-carbonitrile;
1-(2-(4-(2-amino-3-cyano-7-fluorobenzo[b]thiophene-4-yl)-3,5-dimethyl-1H-pyrazol-1-yl)-6-(1-(1-methylpyrrolidin-2-yl)ethoxy)pyrimidin-4-yl)-N-(1-methylcyclopropyl)-1H-pyrazole-3-carboxamide;
5-(6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidin-4-yl)-N,N-dimethylpicolinamide;
2-amino-4-(3,5-dimethyl-1-(6-(1-methyl-6-oxo-1,6-dihydropyridin-3-yl)-2-(1-(1-methylpyrrolidin-2-yl)ethoxy)pyrimidin-4-yl)-1H-pyrazol-4-yl)-7-fluorobenzo[b]thiophene-3-carbonitrile;
4-(6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidin-4-yl)-2-fluoro-N-methylbenzamide;
2-amino-4-(3,5-dimethyl-1-(6-(2-methyl-1-oxoisoindolin-5-yl)-2-(1-(1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(3-methyl-[1,2,4]triazolo[4,3-a]pyridin-6-yl-2-(1-(1-methylpyrrolidin-2-yl)ethoxy)pyrimidin-4-yl)-1H-pyrazol-4-yl)-7-fluorobenzo[b]thiophene-3-carbonitrile;
methyl 2-(6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidin-4-yl)pyrrolidine-1-carboxylate;
2-amino-4-(3,5-dimethyl-1-(2-(1-(1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-4-(3,5-dimethyl-1-(6-(6-methyl-5-oxo-2,6-diazaspiro[3.4]octan-2-yl)-2-(1-(1-methylpyrrolidin-2-yl)ethoxy)pyrimidin-4-yl)-1H-pyrazol-4-yl)-7-fluorobenzo[b]thiophene-3-carbonitrile;
4-(1-(6-(2-oxa-5-azabicyclo[2.2.1]heptan-5-yl)-2-(1-(1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
2-amino-4-(3,5-dimethyl-1-(2-(1-(1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-7-fluoro-4-(1-(6-((3-fluoropyridin-4-yl)oxy)-2-(1-(1-methylpyrrolidin-2-yl)ethoxy)pyrimidin-4-yl)-3,5-dimethyl-1H-pyrazol-4-yl)benzo[b]thiophene-3-carbonitrile;
4-(1-(6-(1-acetylpiperidin-4-yl)-2-(-(1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
2-amino-4-(3,5-dimethyl-1-(4-(1-(methyl-d₃)-1H-pyrazol-4-yl)-6-(1-(1-methylpyrrolidin-2-yl)ethoxy)pyridin-2-yl)-1H-pyrazol-4-yl)-7-fluorobenzo[b]thiophene-3-carbonitrile;
4-(6-(4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide;
4-(6-(4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide;
2-amino-4-(3,5-dimethyl-1-(6-(1-methyl-1H-pyrazol-4-yl)-2-(1-(1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3-chloro-5-methyl-1-(6-(1-methyl-1H-pyrazol-4-yl)-2-(1-(1-methylpyrrolidin-2-yl)ethoxy)pyrimidin-4-yl)-1H-pyrazol-4-yl)-7-fluorobenzo[b]thiophene-3-carbonitrile;
2-amino-7-fluoro-4-(3-(methoxymethyl)-5-methyl-1-(6-(1-methyl-1H-pyrazol-4-yl)-2-(1-(1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-(1-(1-methylpyrrolidin-2-yl)ethoxy)pyrimidin-4-yl)-1H-pyrazol-4-yl)benzo[b]thiophene-3-carbonitrile;
2-amino-4-(3-cyclopropyl-5-methyl-1-(6-(1-methyl-1H-pyrazol-4-yl)-2-(1-(1-methylpyrrolidin-2-yl)ethoxy)pyrimidin-4-yl)-1H-pyrazol-4-yl)-7-fluorobenzo[b]thiophene-3-carbonitrile;
2-amino-4-(3-(difluoromethyl)-5-methyl-1-(6-(1-methyl-1H-pyrazol-4-yl)-2-(1-(1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethyl-3-methyl-1-(6-(1-methyl-1H-pyrazol-4-yl)-2-(1-(1-methylpyrrolidin-2-yl)ethoxy)pyrimidin-4-yl)-1H-pyrazol-4-yl)-7-fluorobenzo[b]thiophene-3-carbonitrile;
2-amino-7-fluoro-4-(3-methyl-1-(6-(1-methyl-1H-pyrazol-4-yl)-2-(1-(1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-4-(3-(hydroxymethyl)-5-methyl-1-(6-(1-methyl-1H-pyrazol-4-yl)-2-(1-(1-methylpyrrolidin-2-yl)ethoxy)pyrimidin-4-yl)-1H-pyrazol-4-yl)benzo[b]thiophene-3-carbonitrile;
2-amino-7-fluoro-4-(5-(hydroxymethyl)-3-methyl-1-(6-(1-methyl-1H-pyrazol-4-yl)-2-(1-(1-methylpyrrolidin-2-yl)ethoxy)pyrimidin-4-yl)-1H-pyrazol-4-yl)benzo[b]thiophene-3-carbonitrile;
4-(6-(4-(2-amino-3-cyano-7-fluorobenzo[b]thiophene-4-yl)-3,5-dimethyl-1H-pyrazol-1-yl)-2-((1-((dimethylamino)methyl)cyclopropyl)methoxy)pyrimidin-4-yl)-N,N-dimethylpiperazine-1-carboxamide;
4-(6-(4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide;
4-(2-(3-acetamidopyrrolidin-1-yl)-6-(4-(2-amino-3-cyano-7-fluorobenzo[b]thiophene-4-yl)-3,5-dimethyl-1H-pyrazol-1-yl)pyrimidin-4-yl)-N,N-dimethylpiperazine-1-carboxamide;
4-(6-(4-(2-amino-3-cyano-7-fluorobenzo[b]thiophene-4-yl)-3,5-dimethyl-1H-pyrazol-1-yl)-2-(1-methylhexahydropyrrolo[3,4-b]pyrrol-5(1H)-yl)pyrimidin-4-yl)-2-fluoro-N-methylbenzamide;
4-(6-(4-(2-amino-3-cyano-7-fluorobenzo[b]thiophene-4-yl)-3,5-dimethyl-1H-pyrazol-1-yl)-2-(1-methyloctahydro-6H-pyrrolo[3,4-b]pyridin-6-yl)pyrimidin-4-yl)-2-fluoro-N-methylbenzamide;
2-amino-4-(5-ethynyl-3-methyl-1-(6-(1-methyl-1H-pyrazol-4-yl)-2-(1-(1-methylpyrrolidin-2-yl)ethoxy)pyrimidin-4-yl)-1H-pyrazol-4-yl)-7-fluorobenzo[b]thiophene-3-carbonitrile;
N-(1-(6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)pyrimidin-4-yl)ethyl)cyclopropanecarboxamide;
2-amino-4-(1-(6-((1-(cyclopropanecarbonyl)azetidin-2-yl)methyl)-2-(1-(1-methylpyrrolidin-2-yl)ethoxy)pyrimidin-4-yl)-3,5-dimethyl-1H-pyrazol-4-yl)-7-fluorobenzo[b]thiophene-3-carbonitrile;
6-(4-(2-amino-3-cyano-7-fluorobenzo[b]thiophene-4-yl)-3,5-dimethyl-1H-pyrazol-1-yl)-2-(1-(1-methylpyrrolidin-2-yl)ethoxy)-N-(1-(pyrimidin-5-yl)ethyl)pyrimidine-4-carboxamide;
2-amino-4-(5-ethyl-3-methyl-1-(6-(2-methyl-1-oxoisoindolin-5-yl)-2-(1-(1-methylpyrrolidin-2-yl)ethoxy)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-(1-methylhexahydropyrrolo[3,4-b]pyrrol-5(1H)-yl)pyrimidin-4-yl)-1H-pyrazol-4-yl)-7-fluorothieno[3,2-c]pyridine-3-carbonitrile;
5-(6-(4-(2-amino-3-cyano-7-fluorothieno[3,2-c]pyridin-4-yl)-5-ethyl-3-methyl-1H-pyrazol-1-yl)-2-(1-(1-methylpyrrolidin-2-yl)ethoxy)pyrimidin-4-yl)-N-methylpicolinamide;
4-(6-(4-(2-amino-3-cyano-7-fluorothieno[3,2-c]pyridin-4-yl)-5-ethyl-3-methyl-1H-pyrazol-1-yl)-2-(1-(1-methylpyrrolidin-2-yl)ethoxy)pyrimidin-4-yl)-2-fluoro-N-methylbenzamide;
4-(6-(4-(2-amino-3-cyano-7-fluorothieno[3,2-c]pyridin-4-yl)-5-ethyl-3-methyl-1H-pyrazol-1-yl)-2-(1-(1-methylpyrrolidin-2-yl)ethoxy)pyrimidin-4-yl)-N-cyclopropyl-2-fluorobenzamide;
5-(6-(4-(2-amino-3-cyano-7-fluorothieno[3,2-c]pyridin-4-yl)-5-ethyl-3-methyl-1H-pyrazol-1-yl)-2-(1-methylhexahydropyrrolo[3,4-b]pyrrol-5(1H)-yl)pyrimidin-4-yl)-3-fluoro-N-methylpicolinamide;
4-(6-(4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-2-fluorobenzamide;
5-(6-(4-(2-amino-3-cyano-7-fluorothieno[3,2-c]pyridin-4-yl)-5-ethyl-3-methyl-1H-pyrazol-1-yl)-2-(1-methylhexahydropyrrolo[3,4-b]pyrrol-5(1H)-yl)pyrimidin-4-yl)-N-cyclopropyl-3-fluoropicolinamide;
2-amino-4-(5-ethyl-1-(6-(2-methyl-1-oxoisoindolin-5-yl)-2-(1-(1-methylpyrrolidin-2-yl)ethoxy)pyrimidin-4-yl)-3-(trifluoromethyl)-1H-pyrazol-4-yl)-7-fluorothieno[3,2-c]pyridine-3-carbonitrile;
2-amino-4-(3,5-diethyl-1-(6-(2-methyl-1-oxoisoindolin-5-yl)-2-(1-(1-methylpyrrolidin-2-yl)ethoxy)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-7-fluorothieno[3,2-c]pyridine-3-carbonitrile;
2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-(6-methyl-3,6-diazabicyclo[3.2.0]heptan-3-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-(1-methyloctahydro-6H-pyrrolo[3,4-b]pyridin-6-yl)pyrimidin-4-yl)-1H-pyrazol-4-yl)-7-fluorothieno[3,2-c]pyridine-3-carbonitrile; and
2-amino-4-(1-(2-(1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
and pharmaceutically acceptable salts thereof.

24. The compound of claim 23, wherein the compound is selected from
4-(1-(4-(4-acetylpiperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(4-acetylpiperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(4-acetylpiperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(4-acetylpiperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(4-acetylpiperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(4-acetylpiperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(4-acetylpiperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(4-acetylpiperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(4-acetylpiperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(4-acetylpiperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(4-acetylpiperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(4-acetylpiperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
1-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpyrrolidine-3-carboxamide;
1-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methylpyrrolidine-3-carboxamide;
1-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methylpyrrolidine-3-carboxamide;
1-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methylpyrrolidine-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpyrrolidine-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methylpyrrolidine-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methylpyrrolidine-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methylpyrrolidine-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpyrrolidine-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methylpyrrolidine-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methylpyrrolidine-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methylpyrrolidine-3-carboxamide;
2-amino-4-(3,5-dimethyl-1-(4-(4-methyl-3-oxopiperazin-1-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(4-methyl-3-oxopiperazin-1-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(4-methyl-3-oxopiperazin-1-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(4-methyl-3-oxopiperazin-1-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(4-methyl-3-oxopiperazin-1-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(4-methyl-3-oxopiperazin-1-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(4-methyl-3-oxopiperazin-1-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(4-methyl-3-oxopiperazin-1-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(4-methyl-3-oxopiperazin-1-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(4-methyl-3-oxopiperazin-1-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(4-methyl-3-oxopiperazin-1-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(4-methyl-3-oxopiperazin-1-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-7-fluoro-4-(1-(4-(4-hydroxy-4-methylpiperidin-1-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-4-(1-(4-(4-hydroxy-4-methylpiperidin-1-yl)-6-((S)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-4-(1-(4-(4-hydroxy-4-methylpiperidin-1-yl)-6-((R)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-4-(1-(4-(4-hydroxy-4-methylpiperidin-1-yl)-6-((R)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(4-hydroxy-4-methylpiperidin-1-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(4-hydroxy-4-methylpiperidin-1-yl)-6-((S)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(4-hydroxy-4-methylpiperidin-1-yl)-6-((R)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(4-hydroxy-4-methylpiperidin-1-yl)-6-((R)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(4-hydroxy-4-methylpiperidin-1-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(4-hydroxy-4-methylpiperidin-1-yl)-6-((S)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(4-hydroxy-4-methylpiperidin-1-yl)-6-((R)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(4-hydroxy-4-methylpiperidin-1-yl)-6-((R)-1-((R)-1-methylpyrrolidin-2-yl)ethoxy)pyrimidin-2-yl)-3,5-dimethyl-1H-pyrazol-4-yl)benzo[b]thiophene-3-carbonitrile;
4-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpiperazine-1-carboxamide;
4-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethylpiperazine-1-carboxamide;
4-(2-(4-(2-amino-3-cyano-7-fluorobenzo[b]thiophene-4-yl)-3,5-dimethyl-1H-pyrazol-1 -yl)-6-((R)-1-((S)-1-methylpyrrolidin-2-yl)ethoxy)pyrimidin-4-yl)-N,N-dimethylpiperazine-1-carboxamide 4-(2-(4-(2-amino-3-cyano-7-fluorobenzo[b]thiophene-4-yl)-3,5-dimethyl-1H-pyrazol-1 -yl)-6-((R)-1-((R)-1-methylpyrrolidin-2-yl)ethoxy)pyrimidin-4-yl)-N,N-dimethylpiperazine-1-carboxamide;
4-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpiperazine-1-carboxamide;
4-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethylpiperazine-1-carboxamide;
4-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethylpiperazine-1-carboxamide;
4-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethylpiperazine-1-carboxamide;
4-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpiperazine-1-carboxamide;
4-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethylpiperazine-1-carboxamide;
4-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethylpiperazine-1-carboxamide;
4-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethylpiperazine-1-carboxamide;
4-(1-(4-(6-acetyl-2,6-diazaspiro[3.3]heptan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(6-acetyl-2,6-diazaspiro[3.3]heptan-2-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(6-acetyl-2,6-diazaspiro[3.3]heptan-2-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(6-acetyl-2,6-diazaspiro[3.3]heptan-2-yl)-6-((R)-1-((R)-1-methlpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.3]heptan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.3]heptan-2-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.3]heptan-2-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.3]heptan-2-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(6-acetyl-2,6-diazaspiro[3.3]heptan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(6-acetyl-2,6-diazaspiro[3.3]heptan-2-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(6-acetyl-2,6-diazaspiro[3.3]heptan-2-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(6-acetyl-2,6-diazaspiro[3.3]heptan-2-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(1-(4-(4-(cyclopropanecarbonyl)piperazin-1-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(4-(cyclopropanecarbonyl)piperazin-1-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(4-(cyclopropanecarbonyl)piperazin-1-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(4-(cyclopropanecarbonyl)piperazin-1-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
Methyl 4-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)piperazine-1-carboxylate;
methyl 4-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)piperazine-1-carboxylate;
methyl 4-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)piperazine-1-carboxylate;
methyl 4-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)piperazine-1-carboxylate;
methyl 4-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)piperazine-1-carboxylate;
methyl 4-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)piperazine-1-carboxylate;
methyl 4-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)piperazine-1-carboxylate;
methyl 4-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)piperazine-1-carboxylate;
methyl 4-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)piperazine-1-carboxylate;
methyl 4-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)piperazine-1-carboxylate;
methyl 4-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)piperazine-1-carboxylate;
methyl 4-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)piperazine-1-carboxylate;
2-amino-4-(3,5-dimethyl-1-(4-(2-methyl-3-oxohexahydroimidazo[1,5-a]pyrazin-7(1H)-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(2-methyl-3-oxohexahydroimidazo[1,5-a]pyrazin-7(1H)-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(2-methyl-3-oxohexahydroimidazo[1,5-a]pyrazin-7(1H)-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(2-methyl-3-oxohexahydroimidazo[1,5-a]pyrazin-7(1H)-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(2-methyl-3-oxohexahydroimidazo[1,5-a]pyrazin-7(1H)-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(2-methyl-3-oxohexahydroimidazo[1,5-a]pyrazin-7(1H)-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(2-methyl-3-oxohexahydroimidazo[1,5-a]pyrazin-7(1H)-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(2-methyl-3-oxohexahydroimidazo[1,5-a]pyrazin-7(1H)-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(2-methyl-3-oxohexahydroimidazo[1,5-a]pyrazin-7(1H)-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(2-methyl-3-oxohexahydroimidazo[1,5-a]pyrazin-7(1H)-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(S)-4-(3,5-dimethyl-1-(4-(2-methyl-3-oxohexahydroimidazo[1,5-a]pyrazin-7(1H)-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(S)-4-(3,5-dimethyl-1-(4-(2-methyl-3-oxohexahydroimidazo[1,5-a]pyrazin-7(1H)-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
4-(1-(4-(6-acetyl-2,6-diazaspiro[3.4]octan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(6-acetyl-2,6-diazaspiro[3.4]octan-2-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(6-acetyl-2,6-diazaspiro[3.4]octan-2-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(6-acetyl-2,6-diazaspiro[3.4]octan-2-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.4]octan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.4]octan-2-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.4]octan-2-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(6-acetyl-2,6-diazaspiro[3.4]octan-2-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(S)-4-(1-(4-(6-acetyl-2,6-diazaspiro[3.4]octan-2-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(S)-4-(1-(4-(6-acetyl-2,6-diazaspiro[3.4]octan-2-)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(6-acetyl-2,6-diazaspiro[3.4]octan-2-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(6-acetyl-2,6-diazaspiro[3.4]octan-2-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-(3-acetyl-3,8-diazabicyclo[3.2.1]octan-8-yl)-6-((S)-1-((S)-1-methylpyrrolidin-2-yl)ethoxy)pyrimidin-2-yl)pyrimidin)-3,5-dimethyl-H-pyrazol-4-yl)-2-amino-7-fluorobenzo[b]thiophene-3-carbonitrile;
4-(1-(4-((1R,5S)-3-acetyl-3,8-diazabicyclo[3.2.1]octan-8-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1R,5S)-3-acetyl-3,8-diazabicyclo[3.2.1]octan-8-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1 S,5R)-3-acetyl-3,8-diazabicyclo[3.2.1]octan-8-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1 S,5R)-3-acetyl-3,8-diazabicyclo[3.2.1]octan-8-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1 S,5R)-3-acetyl-3,8-diazabicyclo[3.2.1]octan-8-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1 S,5R)-3-acetyl-3,8-diazabicyclo[3.2.1]octan-8-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1 S,5R)-3-acetyl-3,8-diazabicyclo[3.2.1]octan-8-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-(3-acetyl-3,8-diazabicyclo[3.2.1]octan-8-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1R,5S)-3-acetyl-3,8-diazabicyclo[3.2.1]octan-8-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1R,5S)-3-acetyl-3,8-diazabicyclo[3.2.1]octan-8-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1 S,5R)-3-acetyl-3,8-diazabicyclo[3.2.1]octan-8-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1 S,5R)-3-acetyl-3,8-diazabicyclo[3.2.1]octan-8-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1 S,5R)-3-acetyl-3,8-diazabicyclo[3.2.1]octan-8-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1 S,5R)-3-acetyl-3,8-diazabicyclo[3.2.1]octan-8-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1 S,5R)-3-acetyl-3,8-diazabicyclo[3.2.1]octan-8-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-(3-acetyl-3,8-diazabicyclo[3.2.1]octan-8-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1R,5S)-3-acetyl-3,8-diazabicyclo[3.2.1]octan-8-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1R,5S)-3-acetyl-3,8-diazabicyclo[3.2.1]octan-8-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1 S,5R)-3-acetyl-3,8-diazabicyclo[3.2.1]octan-8-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1 S,5R)-3-acetyl-3,8-diazabicyclo[3.2.1]octan-8-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1 S,5R)-3-acetyl-3,8-diazabicyclo[3.2.1]octan-8-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1 S,5R)-3-acetyl-3,8-diazabicyclo[3.2.1]octan-8-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1 S,5R)-3-acetyl-3,8-diazabicyclo[3.2.1]octan-8-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((S)-4-acetyl-3-(cyanomethyl)piperazin-1-yl)-6-((S)-1-((S)-1-methlpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((S)-4-acetyl-3-(cyanomethyl)piperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((S)-4-acetyl-3-(cyanomethyl)piperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((R)-4-acetyl-3-(cyanomethyl)piperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((R)-4-acetyl-3-(cyanomethyl)piperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((R)-4-acetyl-3-(cyanomethyl)piperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((R)-4-acetyl-3-(cyanomethyl)piperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((S)-4-acetyl-3-(cyanomethyl)piperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((S)-4-acetyl-3-(cyanomethyl)piperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((S)-4-acetyl-3-(cyanomethyl)piperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((S)-4-acetyl-3-(cyanomethyl)piperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((R)-4-acetyl-3-(cyanomethyl)piperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((R)-4-acetyl-3-(cyanomethyl)piperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((R)-4-acetyl-3-(cyanomethyl)piperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((R)-4-acetyl-3-(cyanomethyl)piperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((S)-4-acetyl-3-(cyanomethyl)piperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((S)-4-acetyl-3-(cyanomethyl)piperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((S)-4-acetyl-3-(cyanomethyl)piperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(S)-4-(1-(4-((S)-4-acetyl-3-(cyanomethyl)piperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((R)-4-acetyl-3-(cyanomethyl)piperazin-1-yl)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((R)-4-acetyl-3-(cyanomethyl)piperazin-1-yl)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((R)-4-acetyl-3-(cyanomethyl)piperazin-1-yl)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((R)-4-acetyl-3-(cyanomethyl)piperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(S)-4-(1-(4-((S)-4-acetyl-3-(cyanomethyl)piperazin-1-yl)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(1-(4-(5,6-dihydroimidazo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydroimidazo[1,5-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydroimidazo[1,5-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydroimidazo[1,5-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydroimidazo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydroimidazo[1,5-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydroimidazo[1,5-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydroimidazo[1,5-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydroimidazo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydroimidazo[1,5-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydroimidazo[1,5-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydroimidazo[1,5-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[4,3-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[4,3-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[4,3-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[4,3-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[4,3-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[4,3-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[4,3-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[4,3-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[4,3-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[4,3-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[4,3-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[4,3-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(3,5-dimethyl-1-(4-((S)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-4-(3,5-dimethyl-1-(4-((S)-1-((R)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-4-(3,5-dimethyl-1-(4-((R)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-4-(3,5-dimethyl-1-(4-((R)-1-((R)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((R)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((S)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((R)-1-methylpyrrolidin-2-yl)ethoxy)-6-(4-(methylsulfonyl)piperazin-1-yl)pyrimidin-2-yl)-1H-pyrazol-4-yl)-7-fluorobenzo[b]thiophene-3-carbonitrile;
2-amino-4-(3,5-dimethyl-1-(4-((S)-1-((S)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-4-(3,5-dimethyl-1-(4-((S)-1-((R)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-4-(3,5-dimethyl-1-(4-((R)-1-((S)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-4-(3,5-dimethyl-1-(4-((R)-1-((R)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-1-methlpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((R)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((S)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((R)-1-methylpyrrolidin-2-yl)ethoxy)-6-(6-oxohexahydropyrrolo[1,2-a]pyrazin-2(1H)-yl)pyrimidin-2-yl)-1H-pyrazol-4-yl)-7-fluorobenzo[b]thiophene-3-carbonitrile;
2-amino-4-(1-(4-(5,6-dihydro-[1,2,4]triazolo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[1,5-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[1,5-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(1-(4-(5,6-dihydro-[1,2,4]triazolo[1,5-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[1,5-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[1,5-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Rₐ)-4-(1-(4-(5,6-dihydro-[1,2,4]triazolo[1,5-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[1,5-a]pyrazin-7(8H)-yl)-6-((S)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[1,5-a]pyrazin-7(8H)-yl)-6-((R)-1-((S)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[1,5-a]pyrazin-7(8H)-yl)-6-((S)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-(Sₐ)-4-(1-(4-(5,6-dihydro-[1,2,4]triazolo[1,5-a]pyrazin-7(8H)-yl)-6-((R)-1-((R)-1-methylpyrrolidin-2-yl)ethoxy)pyrimidin-2-yl)-3,5-dimethyl-1H-pyrazol-4-yl)-7-fluorobenzo[b]thiophene-3-carbonitrile;
2-amino-4-(3,5-dimethyl-1-(4-((S)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-4-(3,5-dimethyl-1-(4-((S)-1-((R)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-4-(3,5-dimethyl-1-(4-((R)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-4-(3,5-dimethyl-1-(4-((R)-1-((R)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((R)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((S)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((R)-1-methylpyrrolidin-2-yl)ethoxy)-6-((pyridin-3-ylmethyl)amino)pyrimidin-2-yl)-1H-pyrazol-4-yl)-7-fluorobenzo[b]thiophene-3-carbonitrile;
2-amino-4-(3,5-dimethyl-1-(4-((S)-1-((S)-1-methylpyrrolidin-2-yl)ethoxy)-6-((1-(pyridin-3-yl)ethyl)amino)pyrimidin-2-yl)-1H-pyrazol-4-yl)-7-fluorobenzo[b]thiophene-3-carbonitrile;
2-amino-4-(3,5-dimethyl-1-(4-((S)-1-((R)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-4-(3,5-dimethyl-1-(4-((S)-1-((S)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-4-(3,5-dimethyl-1-(4-((R)-1-((S)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-4-(3,5-dimethyl-1-(4-((R)-1-((R)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-4-(3,5-dimethyl-1-(4-((R)-1-((S)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-4-(3,5-dimethyl-1-(4-((R)-1-((R)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-4-(3,5-dimethyl-1-(4-((S)-1-((R)-1-methlpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-1-methylpyrrolidin-2-yl)ethoxy)-6-((1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-((1-(pyridin-3-yl)ethyl)amino)pyrimidin-2-yl)-1H-pyrazol-4-yl)-7-fluorobenzo[b]thiophene-3-carbonitrile;
2-amino-(S)-4-(3,5-dimethyl-1-(4-((S)-1-((R)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((S)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((R)-1-methylpyrrolidin-2-yl)ethoxy)-6-(((S)-1-(pyridin-3-yl)ethyl)amino)pyrimidin-2-yl)-1H-pyrazol-4-yl)-7-fluorobenzo[b]thiophene-3-carbonitrile;
2-amino-(S)-4-(3,5-dimethyl-1-(4-((R)-1-((S)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(S)-4-(3,5-dimethyl-1-(4-((R)-1-((R)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((R)-1-methylpyrrolidin-2-yl)ethoxy)-6-(((R)-1-(pyridin-3-yl)ethyl)amino)pyrimidin-2-yl)-1H-pyrazol-4-yl)-7-fluorobenzo[b]thiophene-3-carbonitrile;
4-(1-(4-((1-acetylpiperidin-4-yl)amino)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1-acetylpiperidin-4-yl)amino)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1-acetylpiperidin-4-yl)amino)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1-acetylpiperidin-4-yl)amino)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)amino)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)amino)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)amino)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)amino)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)amino)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)amino)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)amino)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)amino)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(3,5-dimethyl-1-(4-(1-methyl-1H-pyrazol-4-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-4-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-4-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-4-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-4-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-4-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-4-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-4-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-4-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-4-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-4-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-4-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-3-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-3-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-1H-pyrazol-3-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-3-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-3-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-1H-pyrazol-3-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-3-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-3-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-1H-pyrazol-3-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(3-methylisoxazol-5-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(3-methylisoxazol-5-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(3-methylisoxazol-5-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(3-methylisoxazol-5-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(3-methylisoxazol-5-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(3-methylisoxazol-5-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(3-methylisoxazol-5-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(3-methyl isoxazol-5-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(3-methylisoxazol-5-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(3-methylisoxazol-5-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(3-methylisoxazol-5-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(3-methylisoxazol-5-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-6-oxo-1,6-dihydropyridin-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-6-oxo-1,6-dihydropyridin-3-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-6-oxo-1,6-dihydropyridin-3-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-(1-methyl-6-oxo-1,6-dihydropyridin-3-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-6-oxo-1,6-dihydropyridin-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-6-oxo-1,6-dihydropyridin-3-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-6-oxo-1,6-dihydropyridin-3-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Rₐ)-4-(3,5-dimethyl-1-(4-(1-methyl-6-oxo-1,6-dihydropyridin-3-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-6-oxo-1,6-dihydropyridin-3-yl)-6-((S)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-6-oxo-1,6-dihydropyridin-3-yl)-6-((S)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-6-oxo-1,6-dihydropyridin-3-yl)-6-((R)-1-((S)-1-methylpyrrolidin-2-yl)ethoxy)pyrimidin-2-yl)-1H-pyrazol-4-yl)-7-fluorobenzo[b]thiophene-3-carbonitrile;
2-amino-(Sₐ)-4-(3,5-dimethyl-1-(4-(1-methyl-6-oxo-1,6-dihydropyridin-3-yl)-6-((R)-1-((R)-1-methylpyrrolidin-2-yl)ethoxy)pyrimidin-2-yl)-1H-pyrazol-4-yl)-7-fluorobenzo[b]thiophene-3-carbonitrile;
2-amino-4-(3,5-dimethyl-1-(4-((S)-1-((S)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-4-3,5-dimethyl-1-(4-((S)-1-(R)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-4-3,5-dimethyl-1-(4-((R)-1(S)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-4-(3,5-dimethyl-1-(4-((R)-1-((R)-1-methlpyrrolidin-2-)ethoxy)-6-(pyridin-3-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(pyridin-3-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((R)-1-methylpyrrolidin-2-yl)ethoxy)-6-(pyridin-3-yl)pyrimidin-2-yl-1H-pyrazol-4-yl)-7-fluorobenzo[b]thiophene-3-carbonitrile;
2-amino-(Sₐ)-4-(3,5-dimethyl-1-(4-((R)-1-((S)-1-methylpyrrolidin-2-yl)ethoxy)-6-(pyridin-3-yl)pyrimidin-2-yl-1H-pyrazol-4-yl)-7-fluorobenzo[b]thiophene-3-carbonitrile;
2-amino-(Sₐ)-4-(3,5-dimethyl-1-(4-((R)-1-((R)-1-methylpyrrolidin-2-yl)ethoxy)-6-(pyridin-3-yl)pyrimidin-2-yl-1H-pyrazol-4-yl)-7-fluorobenzo[b]thiophene-3-carbonitrile;
2-amino-7-fluoro-4-(1-(4-(6-(2-hydroxypropan-2-yl)pyridin-3-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-4-(1-(4-(6-(2-hydroxypropan-2-yl)pyridin-3-yl)-6-((S)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-4-(1-(4-(6-(2-hydroxypropan-2-yl)pyridin-3-yl)-6-((R)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-4-(1-(4-(6-(2-hydroxypropan-2-yl)pyridin-3-yl)-6-((R)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(6-(2-hydroxypropan-2-yl)pyridin-3-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(6-(2-hydroxypropan-2-yl)pyridin-3-yl)-6-((S)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(6-(2-hydroxypropan-2-yl)pyridin-3-yl)-6-((R)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Rₐ)-4-(1-(4-(6-(2-hydroxypropan-2-yl)pyridin-3-yl)-6-((R)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(6-(2-hydroxypropan-2-yl)pyridin-3-yl)-6-((S)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(6-(2-hydroxypropan-2-yl)pyridin-3-yl)-6-((S)-1-((R)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(6-(2-hydroxypropan-2-yl)pyridin-3-yl)-6-((R)-1-((S)-1-methylpyrrolidin-2-yl)ethoxy)pyrimidin-2-yl)-3,5-dimethyl-1H-pyrazol-4-yl)benzo[b]thiophene-3-carbonitrile;
2-amino-7-fluoro-(Sₐ)-4-(1-(4-(6-(2-hydroxypropan-2-yl)pyridin-3-yl)-6-((R)-1-((R)-1-methylpyrrolidin-2-yl)ethoxy)pyrimidin-2-yl)-3,5-dimethyl-1H-pyrazol-4-yl)benzo[b]thiophene-3-carbonitrile;
5-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpicolinamide;
5-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethylpicolinamide;
5-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethylpicolinamide;
5-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethylpicolinamide;
5-(2-((R)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpicolinamide;
5-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethylpicolinamide;
5-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethylpicolinamide;
5-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethylpicolinamide;
5-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethylpicolinamide;
5-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethylpicolinamide;
5-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethylpicolinamide;
5-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethylpicolinamide;
5-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpicolinamide;
5-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methylpicolinamide;
5-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methylpicolinamide;
5-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methylpicolinamide;
5-(2-((R)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpicolinamide;
5-(2-((R)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methylpicolinamide;
5-(2-((R)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methylpicolinamide;
5-(2-((R)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methylpicolinamide;
5-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methylpicolinamide;
5-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methylpicolinamide;
5-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methylpicolinamide;
5-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methylpicolinamide;
2-amino-4-(3,5-dimethyl-1-(6-((S)-1-((S)-1-methlpyrrolidin-2-yl)ethoxy)-[4,5′-bipyrimidin]-2-yl)-1H-pyrazol-4-yl)-7-fluorobenzo[b]thiophene-3-carbonitrile;
2-amino-4-(3,5-dimethyl-1-(6-((S)-1-((R)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-4-(3,5-dimethyl-1-(6-((R)-1-((S)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-4-(3,5-dimethyl-1-(6-((R)-1-((R)-1-methylpyrrolidin-2-yl)ethoxy)-[4,5′-bipyrimidin]-2-yl-1H-pyrazol-4-yl)-7-fluorobenzo[b]thiophene-3-carbonitrile;
2-amino-(Rₐ)-4-(3,5-dimethyl-1-(6-((S)-1-((S)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Rₐ)-4-(3,5-dimethyl-1-(6-((S)-1-((R)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Rₐ)-4-(3,5-dimethyl-1-(6-((R)-1-((S)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Rₐ)-4-(3,5-dimethyl-1-(6-((R)-1-((R)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Sₐ)-4-(3,5-dimethyl-1-(6-((S)-1-((S)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Sₐ)-4-(3,5-dimethyl-1-(6-((S)-1-((R)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Sₐ)-4-(3,5-dimethyl-1-(6-((R)-1-((S)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-(Sₐ)-4-(3,5-dimethyl-1-(6-(((R)-1-(R)-1-methylpyrrolidin-2-yl)ethoxy)-[4,5′-bipyrimidin]-2-yl)-1H-pyrazol-4-yl)-7-fluorobenzo[b]thiophene-3-carbonitrile;
2-amino-4-(3,5-dimethyl-1-(4-((S)-1-((S)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-4-(3,5-dimethyl-1-(4-((S)-1-((R)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-4-(3,5-dimethyl-1-(4-((R)-1-((S)-1-methlpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-4-(3,5-dimethyl-1-(4-((RR)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(pyridin-4-yl)pyrimidin-2-yl-1H-pyrazol-4-yl)-7-fluorobenzo[b]thiophene-3-carbonitrile;
2-amino-(Sₐ)-4-(3,5-dimethyl-1-(4-((S)-1-((R)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((S)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((R)-1-methylpyrrolidin-2-yl)ethoxy)-6-(pyridin-4-yl)pyrimidin-2-yl)-1H-pyrazol-4-yl)-7-fluorobenzo[b]thiophene-3-carbonitrile;
1-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethyl-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N,N-dimethyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N,N-dimethyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N,N-dimethyl-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N,N-dimethyl-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-4-carboxamide;
1-(2-(4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methyl-1H-pyrazole-4-carboxamide;
1-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methyl-1H-pyrazole-4-carboxamide;
1-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methyl-1H-pyrazole-4-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-4-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methyl-1H-pyrazole-4-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methyl-1H-pyrazole-4-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methyl-1H-pyrazole-4-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-methyl-1H-pyrazole-4-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-methyl-1H-pyrazole-4-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-methyl-1H-pyrazole-4-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-methyl-1H-pyrazole-4-carboxamide;
2-amino-4-(3,5-dimethyl-1-(4-((S)-1-((S)-1-methlpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-4-(3,5-dimethyl-1-(4-((S)-1-((R)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-4-(3,5-dimethyl-1-(4-((R)-1-((S)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-4-(3,5-dimethyl-1-(4-((R)-1-((R)-1-methylprrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((S)-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Rₐ)-4-(3,5-dimethyl-1-(4-((S)-1-((R)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((S)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Rₐ)-4-(3,5-dimethyl-1-(4-((R)-1-((R)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((S)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Sₐ)-4-(3,5-dimethyl-1-(4-((S)-1-((R)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((S)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
2-amino-(Sₐ)-4-(3,5-dimethyl-1-(4-((R)-1-((R)-1-methylpyrrolidin-2-yl)ethoxy)-6-(pyrimidin-5-yloxy)pyrimidin-2-yl)-1H-pyrazol-4-yl)-7-fluorobenzo[b]thiophene-3-carbonitrile;
4-(1-(4-((1-acetylpiperidin-4-yl)oxy)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1-acetylpiperidin-4-yl)oxy)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1-acetylpiperidin-4-yl)oxy)-6-((R)-1-((S)-1-methlpyrrolidin-2-yl)ethoxy)pyrimidin-2-yl)-3,5-dimethyl-1H-pyrazol-4-yl)-2-amino-7-fluorobenzo[b]thiophene-3-carbonitrile;
4-(1-(4-((1-acetylpiperidin-4-yl)oxy)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)oxy)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)oxy)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)oxy)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Rₐ)-4-(1-(4-((1-acetylpiperidin-4-yl)oxy)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)oxy)-6-((S)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)oxy)-6-((S)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)oxy)-6-((R)-1-((S)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
(Sₐ)-4-(1-(4-((1-acetylpiperidin-4-yl)oxy)-6-((R)-1-((R)-1-methylpyrrolidin-2-yl)ethoxy)pyrimidin-2-yl)-3,5-dimethyl-1H-pyrazol-4-yl)-2-amino-7-fluorobenzo[b]thiophene-3-carbonitrile;
2-amino-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(2-((S)-1-((S)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((S)-1-((R)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((R)-1-((S)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((R)-1-((R)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-1-((S)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-1-((R)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((S)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((R)-1-methylpyrrolidin-2-yl)ethoxy)-6-(1-(oxetan-3-yl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(1-(oxetan-3-y)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((R)-1-methylpyrrolidin-2-yl)ethoxy)-6-(1-(oxetan-3-y)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((S)-1-methylpyrrolidin-2-yl)ethoxy)-6-(1-(oxetan-3-y)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((R)-1-methylpyrrolidin-2-yl)ethoxy)-6-(1-(oxetan-3-y)-1H-pyrazol-4-yl)pyrimidin-4-yl)-1H-pyrazol-4-yl)-7-fluorobenzo[b]thiophene-3-carbonitrile;
2-amino-4-(1-(6-(1-(difluoromethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-(difluoromethyl)-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-(difluoromethyl)-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-(difluoromethyl)-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(difluoromethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(difluoromethyl)-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(difluoromethyl)-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(difluoromethyl)-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(difluoromethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(difluoromethyl)-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(difluoromethyl)-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(difluoromethyl)-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(3,5-dimethyl-1-(2-((S)-1-((S)-1-methlpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((S)-1-((R)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((R)-1-((S)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((R)-1-((R)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-1-((S)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-1-((R)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((S)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((R)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(1-(pyridin-3-y)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((R)-1-methylpyrrolidin-2-yl)ethoxy)-6-(1-(pyridin-3-yl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((S)-1-methylpyrrolidin-2-yl)ethoxy)-6-(1-(pyridin-3-y)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((R)-1-methylpyrrolidin-2-yl)ethoxy)-6-(1-(pyridin-3-y)-1H-pyrazol-4-yl)pyrimidin-4-yl)-1H-pyrazol-4-yl)-7-fluorobenzo[b]thiophene-3-carbonitrile;
2-amino-4-(1-(6-(1-benzyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-benzyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-benzyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-benzyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-benzyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-benzyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-benzyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-benzyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-benzyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-benzyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-benzyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-benzyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(3,5-dimethyl-1-(2-((S)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((S)-1-((R)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((R)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-4-(3,5-dimethyl-1-(2-((R)-1-((R)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-4-(S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((R)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((R)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((S)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((R)-1-methylpyrrolidin-2-yl)ethoxy)-6-(1-(pyridin-3-ylmethyl)-1H-pyrazol-4-yl)pyrimidin-4-yl)-1H-pyrazol-4-yl)-7-fluorobenzo[b]thiophene-3-carbonitrile;
2-amino-7-fluoro-4-(1-(6-(5-fluoro-1-methyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-4-(1-(6-(5-fluoro-1-methyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-4-(1-(6-(5-fluoro-1-methyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-4-(1-(6-(5-fluoro-1-methyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-(5-fluoro-1-methyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-(5-fluoro-1-methyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-(5-fluoro-1-methyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-(5-fluoro-1-methyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-(5-fluoro-1-methyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-(5-fluoro-1-methyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-(5-fluoro-1-methyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-(5-fluoro-1-methyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-4-(1-(6-(1,3-dimethyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1,3-dimethyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1,3-dimethyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1,3-dimethyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1,3-dimethyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1,3-dimethyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1,3-dimethyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1,3-dimethyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1,3-dimethyl-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1,3-dimethyl-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1,3-dimethyl-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1,3-dimethyl-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
4-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N-methylpicolinamide;
4-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N-methylpicolinamide;
4-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N-methylpicolinamide;
4-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N-methylpicolinamide;
4-(6-((Rₐ)-4-(2-amino-3-cyano-7-fluorobenzo[b]thiophene-4-yl)-3,5-dimethyl-1H-pyrazol-1-yl)-2-((S)-1-((S)-1-methlpyrrolidin-2-yl)ethoxy)pyrimidin-4-yl)-N-methylpicolinamide;
4-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N-methylpicolinamide;
4-(6-((R)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N-methylpicolinamide;
4-(6-((R)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N-methylpicolinamide;
4-(6-((S)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N-methylpicolinamide;
4-(6-((S)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N-methylpicolinamide;
4-(6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N-methylpicolinamide;
4-(6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N-methylpicolinamide;
2-amino-4-(1-(6-(1-(cyanomethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-(cyanomethyl)-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-(cyanomethyl)-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(1-(cyanomethyl)-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(cyanomethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(cyanomethyl)-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(cyanomethyl)-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(1-(cyanomethyl)-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(cyanomethyl)-1H-pyrazol-4-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(cyanomethyl)-1H-pyrazol-4-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(cyanomethyl)-1H-pyrazol-4-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(1-(cyanomethyl)-1H-pyrazol-4-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(4-(cyclopropanecarbonyl)piperazin-1-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(4-(cyclopropanecarbonyl)piperazin-1-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(4-(cyclopropanecarbonyl)piperazin-1-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-(4-(cyclopropanecarbonyl)piperazin-1-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(4-(cyclopropanecarbonyl)piperazin-1-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(4-(cyclopropanecarbonyl)piperazin-1-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(4-(cyclopropanecarbonyl)piperazin-1-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(6-(4-(cyclopropanecarbonyl)piperazin-1-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(4-(cyclopropanecarbonyl)piperazin-1-yl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(4-(cyclopropanecarbonyl)piperazin-1-yl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(4-(cyclopropanecarbonyl)piperazin-1-yl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(6-(4-(cyclopropanecarbonyl)piperazin-1-yl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
4-(1-(6-(4-acetylpiperazin-1-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(4-acetylpiperazin-1-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(4-acetylpiperazin-1-yl)-2-((R)-1-((S)-1-methlpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(4-acetylpiperazin-1-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-2-((S)-1-((S)-1-methlpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-2-((R)-1-((R)-1-methlpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
5-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Rₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
5-(6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N,N-dimethyl-5,6,7,8-tetrahydro-4H-pyrazolo[1,5-a][1,4]diazepine-2-carboxamide;
1-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-1H-pyrazole-3-carboxamide;
1-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-1H-pyrazole-3-carboxamide;
1-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-1H-pyrazole-3-carboxamide;
1-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-1H-pyrazole-3-carboxamide;
1-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-1H-pyrazole-3-carboxamide;
1-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-1H-pyrazole-3-carboxamide;
1-(6-((Rₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-1H-pyrazole-3-carboxamide;
1-(6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-1H-pyrazole-3-carboxamide;
1-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-1H-pyrazole-3-carboxamide;
1-(6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-1H-pyrazole-3-carboxamide;
1-(6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-1H-pyrazole-3-carboxamide;
1-(6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-1H-pyrazole-3-carboxamide;
4-(1-(6-(4-acetylpiperazin-1-yl)-5-fluoro-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(4-acetylpiperazin-1-yl)-5-fluoro-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(4-acetylpiperazin-1-yl)-5-fluoro-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(4-acetylpiperazin-1-yl)-5-fluoro-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-5-fluoro-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-5-fluoro-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-5-fluoro-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(4-acetylpiperazin-1-yl)-5-fluoro-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-5-fluoro-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-5-fluoro-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-5-fluoro-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(4-acetylpiperazin-1-yl)-5-fluoro-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(4-acetylpiperazin-1-yl)-6-(4-(2-amino-3-cyano-7-fluorobenzo[b]thiophene-4-yl)-3,5-dimethyl-1H-pyrazol-1-yl)-2-((S)-1-((S)-1-methlpyrrolidin-2-yl)ethoxy)pyrimidine-5-carbonitrile;
4-(4-acetylpiperazin-1-yl)-6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidine-5-carbonitrile;
4-(4-acetylpiperazin-1-yl)-6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidine-5-carbonitrile;
4-(4-acetylpiperazin-1-yl)-6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidine-5-carbonitrile;
4-(4-acetylpiperazin-1-yl)-6-((Rₐ)-4-(2-amino-3-cyano-7-fluorobenzo[b]thiophene-4-yl)-3,5-dimethyl-1H-pyrazol-1-yl)-2-((S)-1-((S)-1-methlpyrrolidin-2-yl)ethoxy)pyrimidine-5-carbonitrile;
4-(4-acetylpiperazin-1-yl)-6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidine-5-carbonitrile;
4-(4-acetylpiperazin-1-yl)-6-((Rₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidine-5-carbonitrile;
4-(4-acetylpiperazin-1-yl)-6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidine-5-carbonitrile;
4-(4-acetylpiperazin-1-yl)-6-((S)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidine-5-carbonitrile;
4-(4-acetylpiperazin-1-yl)-6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidine-5-carbonitrile;
4-(4-acetylpiperazin-1-yl)-6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidine-5-carbonitrile;
4-(4-acetylpiperazin-1-yl)-6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidine-5-carbonitrile;
2-amino-4-(1-(4-(4-(cyclopropanecarbonyl)piperazin-1-yl)-6-((S)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-4-(1-(4-(4-(cyclopropanecarbonyl)piperazin-1-yl)-6-((S)-1-((R)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-4-(1-(4-(4-(cyclopropanecarbonyl)piperazin-1-yl)-6-((R)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-4-(1-(4-(4-(cyclopropanecarbonyl)piperazin-1-yl)-6-((R)-1-((R)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((S)-1-((R)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((R)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Rₐ)-4-(1-(4-(4-(cyclopropanecarbonyl)piperazin-1-yl)-6-((R)-1-((R)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((S)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((S)-1-((R)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((R)-1-((S)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-(Sₐ)-4-(1-(4-(4-(cyclopropanecarbonyl)piperazin-1-yl)-6-((R)-1-((R)-1-methylpyrrolidin-2-yl)ethoxy)pyrimidin-2-yl)-3-methyl-1H-pyrazol-4-yl)-7-fluorobenzo[b]thiophene-3-carbonitrile;
2-amino-7-fluoro-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-4-(1-(2-(((2S,7aR)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-4-(1-(2-(((2R,7aR)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Rₐ)-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Rₐ)-4-(1-(2-(((2S,7aR)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Rₐ)-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Rₐ)-4-(1-(2-(((2R,7aR)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Sₐ)-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Sₐ)-4-(1-(2-(((2S,7aR)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Sₐ)-4-(1-(2-(((2R,7aS)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
2-amino-7-fluoro-(Sₐ)-4-(1-(2-(((2R,7aR)-2-fluorotetrahydro-1H-pyrrolizin-7a(5H)-yl)methoxy)-6-(1-methyl-1H-pyrazol-4-yl)pyrimidin-4-yl)-3,5-dimethyl-1H-pyrazol-4-yl)benzo[b]thiophene-3-carbonitrile;
1-(2-(4-(2-amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-methylpyrimidin-4-yl)-1H-pyrazole-3-carboxamide;
2-amino-4-(1-(2-(4-(cyclopropanecarbonyl)piperazin-1-yl)-6-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(2-(4-(cyclopropanecarbonyl)piperazin-1-yl)-6-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(2-(4-(cyclopropanecarbonyl)piperazin-1-yl)-6-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(2-(4-(cyclopropanecarbonyl)piperazin-1-yl)-6-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(2-(4-(cyclopropanecarbonyl)piperazin-1-yl)-6-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(2-(4-(cyclopropanecarbonyl)piperazin-1-yl)-6-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(2-(4-(cyclopropanecarbonyl)piperazin-1-yl)-6-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Rₐ)-4-(1-(2-(4-(cyclopropanecarbonyl)piperazin-1-yl)-6-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(2-(4-(cyclopropanecarbonyl)piperazin-1-yl)-6-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(2-(4-(cyclopropanecarbonyl)piperazin-1-yl)-6-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(2-(4-(cyclopropanecarbonyl)piperazin-1-yl)-6-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-(Sₐ)-4-(1-(2-(4-(cyclopropanecarbonyl)piperazin-1-yl)-6-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
1-(2-(4-(2-amino-3-cyano-7-fluorobenzo[b]thiophene-4-yl)-3,5-dimethyl-1H-pyrazol-1-y)-6-((S)-1-((S)-1-methylpyrrolidin-2-yl)ethoxy)pyrimidin-4-yl)-N-(1-methylcyclopropyl)-1H-pyrazole-3-carboxamide;
1-(2-(4-(2-amino-3-cyano-7-fluorobenzo[b]thiophene-4-yl)-3,5-dimethyl-1H-pyrazol-1-y)-6-((S)-1-((R)-1-methylpyrrolidin-2-yl)ethoxy)pyrimidin-4-yl)-N-(1-methylcyclopropyl)-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-(1-methylcyclopropyl)-1H-pyrazole-3-carboxamide;
1-(2-(4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-(1-methylcyclopropyl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-(1-methylcyclopropyl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-(1-methylcyclopropyl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-(1-methylcyclopropyl)-1H-pyrazole-3-carboxamide;
1-(2-((Rₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-(1-methylcyclopropyl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((S)-1-methylpyrrolidin-2-yl)ethoxy)pyrimidin-4-yl)-N-(1-methylcyclopropyl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((S)-1-((R)-1-methylpyrrolidin-2-yl)ethoxy)pyrimidin-4-yl)-N-(1-methylcyclopropyl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((S)-1-methylpyrrolidin-2-yl)ethoxy)pyrimidin-4-yl)-N-(1-methylcyclopropyl)-1H-pyrazole-3-carboxamide;
1-(2-((Sₐ)-4-(2-amino-3-cyano-7-fluorobenzo[b]thiophene-4-yl)-3,5-dimethyl-1H-pyrazol-1-yl)-6-((R)-1-((R)-1-methylpyrrolidin-2-yl)ethoxy)pyrimidin-4-yl)-N-(1-methylcyclopropyl)-1H-pyrazole-3-carboxamide;
5-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethylpicolinamide;
5-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N,N-dimethylpicolinamide;
5-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N,N-dimethylpicolinamide;
5-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N,N-dimethylpicolinamide;
5-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-N,N-dimethylpicolinamide;
5-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N,N-dimethylpicolinamide;
5-(6-((Rₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N,N-dimethylpicolinamide;
5-(6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N,N-dimethylpicolinamide;
5-(6-((Sₐ)-4-(2-amino-3-cyano-7-fluorobenzo[b]thiophene-4-yl)-3,5-dimethyl-1H-pyrazol-1-yl)-2-((S)-1-((S)-1-methlpyrrolidin-2-yl)ethoxy)pyrimidin-4-yl)-N,N-dimethylpicolinamide;
5-(6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-N,N-dimethylpicolinamide;
5-(6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-N,N-dimethylpicolinamide;
5-(6-((S$)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-N,N-dimethylpicolinamide;
2-amino-4-(3,5-dimethyl-1-(6-(1-methyl-6-oxo-1,6-dihydropyridin-3-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(1-methyl-6-oxo-1,6-dihydropyridin-3-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(1-methyl-6-oxo-1,6-dihydropyridin-3-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(1-methyl-6-oxo-1,6-dihydropyridin-3-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-6-oxo-1,6-dihydropyridin-3-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-6-oxo-1,6-dihydropyridin-3-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-6-oxo-1,6-dihydropyridin-3-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(1-methyl-6-oxo-1,6-dihydropyridin-3-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-6-oxo-1,6-dihydropyridin-3-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-6-oxo-1,6-dihydropyridin-3-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-6-oxo-1,6-dihydropyridin-3-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(1-methyl-6-oxo-1,6-dihydropyridin-3-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
4-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-2-fluoro-N-methylbenzamide;
4-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-2-fluoro-N-methylbenzamide;
4-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-2-fluoro-N-methylbenzamide;
4-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-2-fluoro-N-methylbenzamide;
4-(6-((Rₐ)-4-(2-amino-3-cyano-7-fluorobenzo[b]thiophene-4-yl)-3,5-dimethyl-1H-pyrazol-1-yl)-2-((S)-1-((S)-1-methlpyrrolidin-2-yl)ethoxy)pyrimidin-4-yl)-2-fluoro-N-methylbenzamide;
4-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-2-fluoro-N-methylbenzamide;
4-(6-((R)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-2-fluoro-N-methylbenzamide;
4-(6-((R)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-2-fluoro-N-methylbenzamide;
4-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)-2-fluoro-N-methylbenzamide;
4-(6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)-2-fluoro-N-methylbenzamide;
4-(6-((S)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)-2-fluoro-N-methylbenzamide;
4-(6-((S$)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)-2-fluoro-N-methylbenzamide;
2-amino-4-(3,5-dimethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(3-methyl-[1,2,4]triazolo[4,3-a]pyridin-6-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(3-methyl-[1,2,4]triazolo[4,3-a]pyridin-6-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(3-methyl-[1,2,4]triazolo[4,3-a]pyridin-6-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(3-methyl-[1,2,4]triazolo[4,3-a]pyridin-6-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(3-methyl-[1,2,4]triazolo[4,3-a]pyridin-6-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(3-methyl-[1,2,4]triazolo[4,3-a]pyridin-6-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(3-methyl-[1,2,4]triazolo[4,3-a]pyridin-6-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(3-methyl-[1,2,4]triazolo[4,3-a]pyridin-6-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(3-methyl-[1,2,4]triazolo[4,3-a]pyridin-6-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(3-methyl-[1,2,4]triazolo[4,3-a]pyridin-6-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(3-methyl-[1,2,4]triazolo[4,3-a]pyridin-6-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(3-methyl-[1,2,4]triazolo[4,3-a]pyridin-6-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
methyl (S)-2-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate;
methyl (S)-2-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)pyrrolidine-1-carboxylate;
methyl (S)-2-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)pyrrolidine-1-carboxylate;
methyl (S)-2-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)pyrrolidine-1-carboxylate;
methyl (R)-2-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate;
methyl (R)-2-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)pyrrolidine-1-carboxylate;
methyl (R)-2-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)pyrrolidine-1-carboxylate;
methyl (R)-2-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)pyrrolidine-1-carboxylate;
methyl (S)-2-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate;
methyl (S)-2-(6-((Rₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)pyrrolidine-1-carboxylate;
methyl (S)-2-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)pyrrolidine-1-carboxylate;
methyl (S)-2-(6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)pyrrolidine-1-carboxylate;
methyl (R)-2-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate;
methyl (R)-2-(6-((Rₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)pyrrolidine-1-carboxylate;
methyl (R)-2-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)pyrrolidine-1-carboxylate;
methyl (R)-2-(6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)pyrrolidine-1-carboxylate;
methyl (S)-2-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate;
methyl (S)-2-(6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)pyrrolidine-1-carboxylate;
methyl (S)-2-(6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)pyrrolidine-1-carboxylate;
methyl (S)-2-(6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)pyrrolidine-1-carboxylate;
methyl (R)-2-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)pyrrolidine-1-carboxylate;
methyl (R)-2-(6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)pyrrolidine-1-carboxylate;
methyl (R)-2-(6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)pyrrolidine-1-carboxylate;
methyl (R)-2-(6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)pyrrolidine-1-carboxylate;
2-amino-4-(3,5-dimethyl-1-(2-((S)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-4-(3,5-dimethyl-1-(2-((S)-1-((R)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-4-(3,5-dimethyl-1-(2-((R)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-4-(3,5-dimethyl-1-(2-((R)-1-((R)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Rₐ)-4-(3,5-dimethyl-1l-(2-((S)-1-((R)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Rₐ)-4-(3,5-dimethyl-1l-(2-((R)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((R)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile 2-amino-(Sₐ)-4-(3,5-dimethyl-1-(2-((S)-1-((R)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((S)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((R)-1-methylpyrrolidin-2-yl)ethoxy)-6-(6-oxooctahydro-2H-pyrido[1,2-a]pyrazin-2-yl)pyrimidin-4-yl)-1H-pyrazol-4-yl)-7-fluorobenzo[b]thiophene-3-carbonitrile;
2-amino-4-(3,5-dimethyl-1-(6-(6-methyl-5-oxo-2,6-diazaspiro[3.4]octan-2-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(6-methyl-5-oxo-2,6-diazaspiro[3.4]octan-2-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(6-methyl-5-oxo-2,6-diazaspiro[3.4]octan-2-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3,5-dimethyl-1-(6-(6-methyl-5-oxo-2,6-diazaspiro[3.4]octan-2-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(6-methyl-5-oxo-2,6-diazaspiro[3.4]octan-2-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(6-methyl-5-oxo-2,6-diazaspiro[3.4]octan-2-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(6-methyl-5-oxo-2,6-diazaspiro[3.4]octan-2-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3,5-dimethyl-1-(6-(6-methyl-5-oxo-2,6-diazaspiro[3.4]octan-2-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(S)-4-(3,5-dimethyl-1-(6-(6-methyl-5-oxo-2,6-diazaspiro[3.4]octan-2-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(S)-4-(3,5-dimethyl-1-(6-(6-methyl-5-oxo-2,6-diazaspiro[3.4]octan-2-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(S)-4-(3,5-dimethyl-1-(6-(6-methyl-5-oxo-2,6-diazaspiro[3.4]octan-2-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3,5-dimethyl-1-(6-(6-methyl-5-oxo-2,6-diazaspiro[3.4]octan-2-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
4-(1-(6-((1 S,4S)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-((1 S,4S)-2-oxa-5-azabicyclo[2.2.1]heptan-5-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-((1 S,4S)-2-oxa-5-azabicyclo[2.2.1]heptan-5-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-((1 S,4S)-2-oxa-5-azabicyclo[2.2.1]heptan-5-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-((1R,4R)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-((1R,4R)-2-oxa-5-azabicyclo[2.2.1]heptan-5-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-((1R,4R)-2-oxa-5-azabicyclo[2.2.1]heptan-5-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-((1R,4R)-2-oxa-5-azabicyclo[2.2.1]heptan-5-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1 S,4S)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1 S,4S)-2-oxa-5-azabicyclo[2.2.1]heptan-5-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1 S,4S)-2-oxa-5-azabicyclo[2.2.1]heptan-5-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1 S,4S)-2-oxa-5-azabicyclo[2.2.1]heptan-5-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1R,4R)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1R,4R)-2-oxa-5-azabicyclo[2.2.1]heptan-5-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1R,4R)-2-oxa-5-azabicyclo[2.2.1]heptan-5-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-((1R,4R)-2-oxa-5-azabicyclo[2.2.1]heptan-5-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1 S,4S)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1 S,4S)-2-oxa-5-azabicyclo[2.2.1]heptan-5-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1 S,4S)-2-oxa-5-azabicyclo[2.2.1]heptan-5-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1 S,4S)-2-oxa-5-azabicyclo[2.2.1]heptan-5-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1R,4R)-2-oxa-5-azabicyclo[2.2.1]heptan-5-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1R,4R)-2-oxa-5-azabicyclo[2.2.1]heptan-5-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1R,4R)-2-oxa-5-azabicyclo[2.2.1]heptan-5-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-((1R,4R)-2-oxa-5-azabicyclo[2.2.1]heptan-5-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
2-amino-4-(3,5-dimethyl-1-(2-((S)-1-((S)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-4-(3,5-dimethyl-1-(2-((S)-1-((R)-1-methylpyrrolidin-2-yl)ethoxy)-6-(pyridin-4-yloxy)pyrimidin-4-y)-1H-pyrazol-4-yl)-7-fluorobenzo[b]thiophene-3-carbonitrile;
2-amino-4-(3,5-dimethyl-1-(2-((R)-1-((S)-1-methylpyrrolidin-2-yl)ethoxy)-6-(pyridin-4-yloxy)pyrimidin-4-y)-1H-pyrazol-4-yl)-7-fluorobenzo[b]thiophene-3-carbonitrile;
2-amino-4-(3,5-dimethyl-1-(2-((R)-1-((R)-1-methylpyrrolidin-2-yl)ethoxy)-6-(pyridin-4-yloxy)pyrimidin-4-y)-1H-pyrazol-4-yl)-7-fluorobenzo[b]thiophene-3-carbonitrile;
2-amino-(Rₐ)-4-(3,5-dimethyl-1-(2-((S)-1-((S)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-(Rₐ)-4-(3,5-dimethyl-1-(2-((S)-1-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-(Rₐ)-4-(3,5-dimethyl-1-(2-((R)-1-((R)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-(Sₐ)-4-(3,5-dimethyl-1-(2-((S)-1-((S)-1-methylpyrrolidin-2-yl)ethoxy)-6-(pyridin-4-yloxy)pyrimidin-4-y)-1H-pyrazol-4-yl)-7-fluorobenzo[b]thiophene-3-carbonitrile;
2-amino-(Sₐ)-4-(3,5-dimethyl-1-(2-((S)-1-((R)-1-methylpyrrolidin-2-yl)ethoxy)-6-(pyridin-4-yloxy)pyrimidin-4-y)-1H-pyrazol-4-yl)-7-fluorobenzo[b]thiophene-3-carbonitrile;
2-amino-(Sₐ)-4-(3,5-dimethyl-1-(2-((R)-1-((S)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-(Sₐ)-4-(3,5-dimethyl-1-(2-((R)-1-((R)-1-methylpyrrolidin-2-yl)ethoxy)-6-(pyridin-4-yloxy)pyrimidin-4-yl)-1H-pyrazol-4-yl)-7-fluorobenzo[b]thiophene-3-carbonitrile;
2-amino-7-fluoro-4-(1-(6-((3-fluoropyridin-4-yl)oxy)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-4-(1-(6-((3-fluoropyridin-4-yl)oxy)-2-((S)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-4-(1-(6-((3-fluoropyridin-4-)oxy)-2-((R)-1-((S)-1-methlpyrrolidin-2-yl)ethoxy)pyrimidin-4-yl)-3,5-dimethyl-1H-pyrazol-4-yl)benzo[b]thiophene-3-carbonitrile;
2-amino-7-fluoro-4-(1-(6-((3-fluoropyridin-4-yl)oxy)-2-((R)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-((3-fluoropyridin-4-yl)oxy)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-((3-fluoropyridin-4-yl)oxy)-2-((S)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-((3-fluoropyridin-4-yl)oxy)-2-((R)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Rₐ)-4-(1-(6-((3-fluoropyridin-4-yl)oxy)-2-((R)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-((3-fluoropyridin-4-yl)oxy)-2-((S)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-((3-fluoropyridin-4-yl)oxy)-2-((S)-1-((R)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-((3-fluoropyridin-4-yl)oxy)-2-((R)-1-((S)-1-methylpyrrolidin-2-yl)ethoxy)pyrimidin-4-yl)-3,5-dimethyl-1H-pyrazol-4-yl)benzo[b]thiophene-3-carbonitrile;
2-amino-7-fluoro-(Sₐ)-4-(1-(6-((3-fluoropyridin-4-yl)oxy)-2-((R)-1-((R)-1-methylpyrrolidin-2-yl)ethoxy)pyrimidin-4-yl)-3,5-dimethyl-1H-pyrazol-4-yl)benzo[b]thiophene-3-carbonitrile;
4-(1-(6-(1-acetylpiperidin-4-yl)-2-((S)-1-((S)-1-methlpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(1-acetylpiperidin-4-yl)-2-((S)-1-((R)-1-methlpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(1-acetylpiperidin-4-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
4-(1-(6-(1-acetylpiperidin-4-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(1-acetylpiperidin-4-yl)-2-((S)-1-((S)-1-methlpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(1-acetylpiperidin-4-yl)-2-((S)-1-((R)-1-methlpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(1-acetylpiperidin-4-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Rₐ)-4-(1-(6-(1-acetylpiperidin-4-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(1-acetylpiperidin-4-yl)-2-((S)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(1-acetylpiperidin-4-yl)-2-((S)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(1-acetylpiperidin-4-yl)-2-((R)-1-((S)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
(Sₐ)-4-(1-(6-(1-acetylpiperidin-4-yl)-2-((R)-1-((R)-1-methylpyrrolidin-2-yl)ethoxy)pyrimidin-4-yl)-3,5-dimethyl-1H-pyrazol-4-yl)-2-amino-7-fluorobenzo[b]thiophene-3-carbonitrile;
2-amino-4-(3,5-dimethyl-1-(4-(1-(methyl-d)-1H-pyrazol-4-yl)-6-((S)-1-((S)-1-methylpyrrolidin-2-yl)ethoxy)pyridin-2-yl)-1H-pyrazol-4-yl)-7-fluorobenzo[b]thiophene-3-carbonitrile;
2-amino-4-(3,5-dimethyl-1-(4-(1-(methyl-d₃)-1H-pyrazol-4-yl)-6-((S)-1-((R)-1-methylpyrrolidin-2-yl)ethoxy)pyridin-2-yl)-1H-pyrazol-4-yl)-7-fluorobenzo[b]thiophene-3-carbonitrile;
2-amino-4-(3,5-dimethyl-1-(4-(1-(methyl-d₃)-1H-pyrazol-4-yl)-6-((R)-1-((S)-1-methylpyrrolidin-2-yl)ethoxy)pyridin-2-yl)-1H-pyrazol-4-yl)-7-fluorobenzo[b]thiophene-3-carbonitrile;
2-amino-4-(3,5-dimethyl-1-(4-(1-(methyl-d₃)-1H-pyrazol-4-yl)-6-((R)-1-((R)-1-methylpyrrolidin-2-yl)ethoxy)pyridin-2-yl)-1H-pyrazol-4-yl)-7-fluorobenzo[b]thiophene-3-carbonitrile;
2-amino-(Rₐ)-4-(3,5-dimethyl-1-(4-(1-(methyl-d₃)-1H-pyrazol-4-yl)-6-((S)-1-((S)-1-methylpyrrolidin-2-yl)ethoxy)pyridin-2-yl)-1H-pyrazol-4-yl)-7-fluorobenzo[b]thiophene-3-carbonitrile;
2-amino-(Rₐ)-4-(3,5-dimethyl-1-(4-(1-(methyl-d₃)-1H-pyrazol-4-yl)-6-((S)-1-((R)-1-methylpyrrolidin-2-yl)ethoxy)pyridin-2-yl)-1H-pyrazol-4-yl)-7-fluorobenzo[b]thiophene-3-carbonitrile;
2-amino-(Rₐ)-4-(3,5-dimethyl-1-(4-(1-(methyl-d₃)-1H-pyrazol-4-yl)-6-((R)-1-((S)-1-methylpyrrolidin-2-yl)ethoxy)pyridin-2-yl)-1H-pyrazol-4-yl)-7-fluorobenzo[b]thiophene-3-carbonitrile;
2-amino-(Rₐ)-4-(3,5-dimethyl-1-(4-(1-(methyl-d₃)-1H-pyrazol-4-yl)-6-((R)-1-((R)-1-methylpyrrolidin-2-yl)ethoxy)pyridin-2-yl)-1H-pyrazol-4-yl)-7-fluorobenzo[b]thiophene-3-carbonitrile;
2-amino-(Sₐ)-4-(3,5-dimethyl-1-(4-(1-(methyl-d₃)-1H-pyrazol-4-yl)-6-((S)-1-((S)-1-methylpyrrolidin-2-yl)ethoxy)pyridin-2-yl)-1H-pyrazol-4-yl)-7-fluorobenzo[b]thiophene-3-carbonitrile;
2-amino-(Sₐ)-4-(3,5-dimethyl-1-(4-(1-(methyl-d₃)-1H-pyrazol-4-yl)-6-((S)-1-((R)-1-methylpyrrolidin-2-yl)ethoxy)pyridin-2-yl)-1H-pyrazol-4-yl)-7-fluorobenzo[b]thiophene-3-carbonitrile;
2-amino-(Sₐ)-4-(3,5-dimethyl-1-(4-(1-(methyl-d₃)-1H-pyrazol-4-yl)-6-((R)-1-((S)-1-methylpyrrolidin-2-yl)ethoxy)pyridin-2-yl)-1H-pyrazol-4-yl)-7-fluorobenzo[b]thiophene-3-carbonitrile;
2-amino-(Sₐ)-4-(3,5-dimethyl-1-(4-(1-(methyl-d₃)-1H-pyrazol-4-yl)-6-((R)-1-((R)-1-methylpyrrolidin-2-yl)ethoxy)pyridin-2-yl)-1H-pyrazol-4-yl)-7-fluorobenzo[b]thiophene-3-carbonitrile;
(S)-4-(6-(4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide;
(R)-4-(6-(4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide;
(S)-4-(6-((Rₐ)-4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide;
(R)-4-(6-((Sₐ)-4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide;
(S)-4-(6-((Sₐ)-4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide;
(R)-4-(6-((Rₐ)-4-(2-amino-3-cyano-7-fluorobenzo[b]thiophene-4-yl)-3,5-dimethyl-1H-pyrazol-1-yl)-2-((1-methylpyrrolidin-2-yl)methoxy)pyrimidin-4-yl)-N,N-dimethylpiperazine-1-carboxamide;
(S)-4-(6-(4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide;
(R)-4-(6-(4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide;
(S)-4-(6-((Rₐ)-4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide;
(S)-4-(6-((Sₐ)-4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide;
(R)-4-(6-((Rₐ)-4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide;
(R)-4-(6-((Sₐ)-4-(2-amino-3-cyano-7-fluorobenzo[b]thiophene-4-yl)-3,5-dimethyl-1H-pyrazol-1-yl)-2-((1-(dimethylamino)propan-2-yl)oxy)pyrimidin-4-yl)-N,N-dimethylpiperazine-1-carboxamide;
2-amino-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3,5-dimethyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3,5-dimethyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3,5-dimethyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Rₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Rₐ)-4-(3,5-dimethyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Rₐ)-4-(3,5-dimethyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Rₐ)-4-(3,5-dimethyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Sₐ)-4-(3,5-dimethyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Sₐ)-4-(3,5-dimethyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Sₐ)-4-(3,5-dimethyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-(Sₐ)-4-(3,5-dimethyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3-chloro-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-chloro-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-chloro-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-chloro-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-chloro-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-chloro-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(R)-4-(3-chloro-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(R)-4-(3-chloro-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-chloro-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-chloro-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-chloro-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-chloro-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-7-fluoro-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile 2-amino-7-fluoro-(Rₐ)-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(methoxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(methoxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(methoxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(methoxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(methoxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(methoxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(methoxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(methoxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-4-(3-cyclopropyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-cyclopropyl-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-cyclopropyl-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-cyclopropyl-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-cyclopropyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-cyclopropyl-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-cyclopropyl-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-cyclopropyl-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-cyclopropyl-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-cyclopropyl-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-cyclopropyl-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-cyclopropyl-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-(difluoromethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-(difluoromethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-(difluoromethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(3-(difluoromethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(R)-4-(3-(difluoromethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(R)-4-(3-(difluoromethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-(difluoromethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(3-(difluoromethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-(difluoromethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-(difluoromethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-(difluoromethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(3-(difluoromethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethyl-3-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethyl-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(5-ethyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(5-ethyl-3-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(5-ethyl-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Rₐ)-4-(5-ethyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(5-ethyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(5-ethyl-3-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(5-ethyl-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-(Sₐ)-4-(5-ethyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-7-fluoro-4-(3-methyl-1-(6-(1-methyl-1H-pyrazol-4-yl)-2-((S)-1-((S)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-4-(3-methyl-1-(6-(1-methyl-1H-pyrazol-4-yl)-2-((S)-1-((R)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-4-(3-methyl-1-(6-(1-methyl-1H-pyrazol-4-yl)-2-((R)-1-((S)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-4-(3-methyl-1-(6-(1-methyl-1H-pyrazol-4-yl)-2-((R)-1-((R)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(R)-4-(3-methyl-1-(6-(1-methyl-1H-pyrazol-4-yl)-2-((S)-1-((S)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(R)-4-(3-methyl-1-(6-(1-methyl-1H-pyrazol-4-yl)-2-((S)-1-((R)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(R)-4-(3-methyl-1-(6-(1-methyl-1H-pyrazol-4-yl)-2-((R)-1-((S)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(Rₐ)-4-(3-methyl-1-(6-(1-methyl-1H-pyrazol-4-yl)-2-((R)-1-((R)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(Sₐ)-4-(3-methyl-1-(6-(1-methyl-1H-pyrazol-4-yl)-2-((S)-1-((S)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(Sₐ)-4-(3-methyl-1-(6-(1-methyl-1H-pyrazol-4-yl)-2-((S)-1-((R)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(Sₐ)-4-(3-methyl-1-(6-(1-methyl-1H-pyrazol-4-yl)-2-((R)-1-((S)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-(Sₐ)-4-(3-methyl-1-(6-(1-methyl-1H-pyrazol-4-yl)-2-((R)-1-((R)-1-methylpyrrolidin-2-yl)ethoxy)pyrimidin-4-yl)-5-vinyl-1H-pyrazol-4-yl)benzo[b]thiophene-3-carbonitrile;
2-amino-7-fluoro-4-(3-(hydroxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(hydroxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(hydroxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(3-(hydroxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(hydroxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(hydroxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(hydroxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(3-(hydroxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(hydroxymethyl)-5-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(hydroxymethyl)-5-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(hydroxymethyl)-5-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(3-(hydroxymethyl)-5-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-4-(5-(hydroxymethyl)-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(5-(hydroxymethyl)-3-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-4-(5-(hydroxymethyl)-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-4-(5-(hydroxymethyl)-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(5-(hydroxymethyl)-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(5-(hydroxymethyl)-3-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(5-(hydroxymethyl)-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Rₐ)-4-(5-(hydroxymethyl)-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(5-(hydroxymethyl)-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(5-(hydroxymethyl)-3-methyl-1-(6-(1-methyl-1H-pyrazol-4-yl)-2-((S)-1-((R)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(5-(hydroxymethyl)-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)benzo[b]thiophene-3-carbonitrile;
2-amino-7-fluoro-(Sₐ)-4-(5-(hydroxymethyl)-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)benzo[b]thiophene-3-carbonitrile;
4-(6-((Rₐ)-4-(2-amino-3-cyano-7-fluorobenzo[b]thiophene-4-yl)-3,5-dimethyl-1H-pyrazol-1-yl)-2-((1-((dimethylamino)methyl)cyclopropyl)methoxy)pyrimidin-4-yl)-N,N-dimethylpiperazine-1-carboxamide;
4-(6-((Sₐ)-4-(2-amino-3-cyano-7-fluorobenzo[b]thiophene-4-yl)-3,5-dimethyl-1H-pyrazol-1-yl)-2-((1-((dimethylamino)methyl)cyclopropyl)methoxy)pyrimidin-4-yl)-N,N-dimethylpiperazine-1-carboxamide;
(S)-4-(6-((Rₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide;
(S)-4-(6-((Sₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide;
(R)-4-(6-((Rₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide;
(R)-4-(6-((Sₐ)-4-(2-amino-3-cyano-7-fluorobenzo[b]thiophene-4-yl)-3,5-dimethyl-1H-pyrazol-1-yl)-2-(3-(dimethylamino)pyrrolidin-1-yl)pyrimidin-4-yl)-N,N-dimethylpiperazine-1-carboxamide;
4-(2-(3-acetamidopyrrolidin-1-yl)-6-((Rₐ)-4-(2-amino-3-cyano-7-fluorobenzo[b]thiophene-4-yl)-3,5-dimethyl-1H-pyrazol-1-yl)pyrimidin-4-yl)-N,N-dimethylpiperazine-1-carboxamide;
4-(2-(3-acetamidopyrrolidin-1-yl)-6-((Sₐ)-4-(2-amino-3-cyano-7-fluorobenzo[b]thiophene-4-yl)-3,5-dimethyl-1H-pyrazol-1-yl)pyrimidin-4-yl)-N,N-dimethylpiperazine-1-carboxamide;
4-(6-(4-(2-amino-3-cyano-7-fluorobenzo[b]thiophene-4-yl)-3,5-dimethyl-1H-pyrazol-1-yl)-2-((3aS,6aS)-1-methylhexahydropyrrolo[3,4-b]pyrrol-5(1H)-yl)pyrimidin-4-yl)-2-fluoro-N-methylbenzamide;
4-(6-(4-(2-amino-3-cyano-7-fluorobenzo[b]thiophene-4-yl)-3,5-dimethyl-1H-pyrazol-1-yl)-2-((3aR,6aR)-1-methylhexahydropyrrolo[3,4-b]pyrrol-5(1H)-yl)pyrimidin-4-yl)-2-fluoro-N-methylbenzamide;
4-(6-((R)-4-(2-amino-3-cyano-7-fluorobenzo[b]thiophene-4-yl)-3,5-dimethyl-1H-pyrazol-1-yl)-2-((3aS,6aS)-1-methylhexahydropyrrolo[3,4-b]pyrrol-5(1H)-yl)pyrimidin-4-yl)-2-fluoro-N-methylbenzamide;
4-(6-((S$)-4-(2-amino-3-cyano-7-fluorobenzo[b]thiophene-4-yl)-3,5-dimethyl-1H-pyrazol-1-yl)-2-((3aS,6aS)-1-methylhexahydropyrrolo[3,4-b]pyrrol-5(1H)-yl)pyrimidin-4-yl)-2-fluoro-N-methylbenzamide;
4-(6-((R)-4-(2-amino-3-cyano-7-fluorobenzo[b]thiophene-4-yl)-3,5-dimethyl-1H-pyrazol-1-yl)-2-((3aR,6aR)-1-methylhexahydropyrrolo[3,4-b]pyrrol-5(1H)-yl)pyrimidin-4-yl)-2-fluoro-N-methylbenzamide;
4-(6-((Sₐ)-4-(2-amino-3-cyano-7-fluorobenzo[b]thiophene-4-yl)-3,5-dimethyl-1H-pyrazol-1-yl)-2-((3aR,6aR)-1-methylhexahydropyrrolo[3,4-b]pyrrol-5(1H)-yl)pyrimidin-4-yl)-2-fluoro-N-methylbenzamide;
4-(6-(4-(2-amino-3-cyano-7-fluorobenzo[b]thiophene-4-yl)-3,5-dimethyl-1H-pyrazol-1-yl)-2-((4aS,7aS)-1-methyloctahydro-6H-pyrrolo[3,4-b]pyridin-6-yl)pyrimidin-4-yl)-2-fluoro-N-methylbenzamide;
4-(6-(4-(2-amino-3-cyano-7-fluorobenzo[b]thiophene-4-yl)-3,5-dimethyl-1H-pyrazol-1-yl)-2-((4aS,7aS)-1-methyloctahydro-6H-pyrrolo[3,4-b]pyridin-6-yl)pyrimidin-4-yl)-2-fluoro-N-methylbenzamide;
4-(6-((R)-4-(2-amino-3-cyano-7-fluorobenzo[b]thiophene-4-yl)-3,5-dimethyl-1H-pyrazol-1-yl)-2-((4aS,7aS)-1-methyloctahydro-6H-pyrrolo[3,4-b]pyridin-6-yl)pyrimidin-4-yl)-2-fluoro-N-methylbenzamide;
4-(6-((S$)-4-(2-amino-3-cyano-7-fluorobenzo[b]thiophene-4-yl)-3,5-dimethyl-1H-pyrazol-1-yl)-2-((4aS,7aS)-1-methyloctahydro-6H-pyrrolo[3,4-b]pyridin-6-yl)pyrimidin-4-yl)-2-fluoro-N-methylbenzamide;
4-(6-((Rₐ)-4-(2-amino-3-cyano-7-fluorobenzo[b]thiophene-4-yl)-3,5-dimethyl-1H-pyrazol-1-yl)-2-((4aR,7aR)-1-methyloctahydro-6H-pyrrolo[3,4-b]pyridin-6-yl)pyrimidin-4-yl)-2-fluoro-N-methylbenzamide;
4-(6-((Sₐ)-4-(2-amino-3-cyano-7-fluorobenzo[b]thiophene-4-yl)-3,5-dimethyl-1H-pyrazol-1-yl)-2-((4aR,7aR)-1-methyloctahydro-6H-pyrrolo[3,4-b]pyridin-6-yl)pyrimidin-4-yl)-2-fluoro-N-methylbenzamide;
2-amino-4-(5-ethynyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethynyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethynyl-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethynyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-7-fluorobenzo[b]thiophene-3-carbonitrile;
2-amino-4-(5-ethynyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-(Rₐ)-7-fluorobenzo[b]thiophene-3-carbonitrile;
2-amino-4-(5-ethynyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-(Rₐ)-7-fluorobenzo[b]thiophene-3-carbonitrile;
2-amino-4-(5-ethynyl-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-(Rₐ)-7-fluorobenzo[b]thiophene-3-carbonitrile;
2-amino-4-(5-ethynyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-(Rₐ)-7-fluorobenzo[b]thiophene-3-carbonitrile;
2-amino-4-(5-ethynyl-3-methyl-1-(6-(1-methyl-1H-pyrazol-4-yl)-2-((S)-1-((S)-1-methylpyrrolidin-2-yl)ethoxy)pyrimidin-4-yl)-1H-pyrazol-4-yl)-(Sₐ)-7-fluorobenzo[b]thiophene-3-carbonitrile;
2-amino-4-(5-ethynyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-(Sₐ)-7-fluorobenzo[b]thiophene-3-carbonitrile;
2-amino-4-(5-ethynyl-3-methyl-1-(6-(1-methyl-1H-pyrazol-4-yl)-2-((R)-1-((S)-1-methylpyrrolidin-2-yl)ethoxy)pyrimidin-4-yl)-1H-pyrazol-4-yl)-(Sₐ)-7-fluorobenzo[b]thiophene-3-carbonitrile;
2-amino-4-(5-ethynyl-3-methyl-1-(6-(1-methyl-1H-pyrazol-4-yl)-2-((R)-1-((R)-1-methylpyrrolidin-2-yl)ethoxy)pyrimidin-4-yl)-1H-pyrazol-4-yl)-(Sₐ)-7-fluorobenzo[b]thiophene-3-carbonitrile;
N-(1-(6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide;
N-(1-(6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)ethyl)cyclopropanecarboxamide;
N-(1-(6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)ethyl)cyclopropanecarboxamide;
N-(1-(6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)ethyl)cyclopropanecarboxamide;
N-(1-(6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide;
N-(1-(6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)ethyl)cyclopropanecarboxamide;
N-(1-(6-((Rₐ)-4-(2-amino-3-cyano-7-fluorobenzo[b]thiophene-4-yl)-3,5-dimethy-1H-pyrazol-1-yl)-2-((R)-1-((S)-1-methylpyrrolidin-2-yl)ethoxy)pyrimidin-4-yl)ethyl)cyclopropanecarboxamide;
N-(1-(6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)ethyl)cyclopropanecarboxamide;
N-(1-(6-((Sₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)pyrimidin-4-yl)ethyl)cyclopropanecarboxamide;
N-(1-(6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)pyrimidin-4-yl)ethyl)cyclopropanecarboxamide;
N-(1-(6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)pyrimidin-4-yl)ethyl)cyclopropanecarboxamide;
N-(1-(6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)pyrimidin-4-yl)ethyl)cyclopropanecarboxamide;
2-amino-4-(1-(6-((1-(cyclopropanecarbonyl)azetidin-2-yl)methyl)-2-((S)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-((1-(cyclopropanecarbonyl)azetidin-2-yl)methyl)-2-((R)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-((1-(cyclopropanecarbonyl)azetidin-2-yl)methyl)-2-((S)-1-((R)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-((1-(cyclopropanecarbonyl)azetidin-2-yl)methyl)-2-((R)-1-((S)-1-methylpyrrolidin-2-yl)ethoxy)pyrimidin-4-yl)-3,5-dimethyl-1H-pyrazol-4-yl)-7-fluorobenzo[b]thiophene-3-carbonitrile;
2-amino-4-(1-(6-((1-(cyclopropanecarbonyl)azetidin-2-yl)methyl)-2-((S)-1-((S)-1-methylpyrrolidin-2-yl)ethoxy)pyrimidin-4-yl)-3,5-dimethyl-1H-pyrazol-4-yl)-(Rₐ)-7-fluorobenzo[b]thiophene-3-carbonitrile;
2-amino-4-(1-(6-((1-(cyclopropanecarbonyl)azetidin-2-yl)methyl)-2-((R)-1-((R)-1-methylpyrrolidin-2-yl)ethoxy)pyrimidin-4-yl)-3,5-dimethyl-1H-pyrazol-4-yl)-(Rₐ)-7-fluorobenzo[b]thiophene-3-carbonitrile;
2-amino-4-(1-(6-((1-(cyclopropanecarbonyl)azetidin-2-yl)methyl)-2-((S)-1-((R)-1-methylpyrrolidin-2-yl)ethoxy)pyrimidin-4-yl)-3,5-dimethyl-1H-pyrazol-4-yl)-(Rₐ)-7-fluorobenzo[b]thiophene-3-carbonitrile;
2-amino-4-(1-(6-((1-(cyclopropanecarbonyl)azetidin-2-yl)methyl)-2-((R)-1-((S)-1-methylpyrrolidin-2-yl)ethoxy)pyrimidin-4-yl)-3,5-dimethyl-1H-pyrazol-4-yl)-(Rₐ)-7-fluorobenzo[b]thiophene-3-carbonitrile;
2-amino-4-(1-(6-((1-(cyclopropanecarbonyl)azetidin-2-yl)methyl)-2-((S)-1-((S)-1-methylpyrrolidin-2-yl)ethoxy)pyrimidin-4-yl)-3,5-dimethyl-1H-pyrazol-4-yl)-(Sₐ)-7-fluorobenzo[b]thiophene-3-carbonitrile;
2-amino-4-(1-(6-((1-(cyclopropanecarbonyl)azetidin-2-yl)methyl)-2-((R)-1-((R)-1-methylpyrrolidin-2-yl)ethoxy)pyrimidin-4-yl)-3,5-dimethyl-1H-pyrazol-4-yl)-(Sₐ)-7-fluorobenzo[b]thiophene-3-carbonitrile;
2-amino-4-(1-(6-((1-(cyclopropanecarbonyl)azetidin-2-yl)methyl)-2-((S)-1-((R)-1-methylpyrrolidin-2-yl)ethoxy)pyrimidin-4-yl)-3,5-dimethyl-1H-pyrazol-4-yl)-(Sₐ)-7-fluorobenzo[b]thiophene-3-carbonitrile;
2-amino-4-(1-(6-((1-(cyclopropanecarbonyl)azetidin-2-yl)methyl)-2-((R)-1-((S)-1-methylpyrrolidin-2-yl)ethoxy)pyrimidin-4-yl)-3,5-dimethyl-1H-pyrazol-4-yl)-(Sₐ)-7-fluorobenzo[b]thiophene-3-carbonitrile;
6-(4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-(1-(pyrimidin-5-yl)ethyl)pyrimidine-4-carboxamide;
6-(4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)-N-(1-(pyrimidin-5-yl)ethyl)pyrimidine-4-carboxamide;
6-(4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)-N-(1-(pyrimidin-5-yl)ethyl)pyrimidine-4-carboxamide;
6-(4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)-N-(1-(pyrimidin-5-yl)ethyl)pyrimidine-4-carboxamide;
6-((Rₐ)-4-(2-amino-3-cyano-7-fluorobenzo[b]thiophene-4-yl)-3,5-dimethyl-1H-pyrazol-1-yl)-2-((S)-1-((S)-1-methylpyrrolidin-2-yl)ethoxy)-N-(1-(pyrimidin-5-yl)ethyl)pyrimidine-4-carboxamide;
6-((Rₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)-N-(1-(pyrimidin-5-yl)ethyl)pyrimidine-4-carboxamide;
6-((Rₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)-N-(1-(pyrimidin-5-yl)ethyl)pyrimidine-4-carboxamide;
6-((Rₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)-N-(1-(pyrimidin-5-yl)ethyl)pyrimidine-4-carboxamide;
6-((Sₐ)-4-(2-amino-3-cyano-7-fluorobenzo[b]thiophene-4-yl)-3,5-dimethyl-1H-pyrazol-1-yl)-2-((S)-1-((S)-1-methlpyrrolidin-2-yl)ethoxy)-N-(1-(pyrimidin-5-yl)ethyl)pyrimidine-4-carboxamide;
6-((Sₐ)-4-(2-amino-3-cyano-7-fluorobenzo[b]thiophene-4-yl)-3,5-dimethyl-1H-pyrazol-1-yl)-2-((R)-1-((R)-1-methylpyrrolidin-2-yl)ethoxy)-N-(1-(pyrimidin-5-yl)ethyl)pyrimidine-4-carboxamide;
6-((Sₐ)-4-(2-amino-3-cyano-7-fluorobenzo[b]thiophene-4-yl)-3,5-dimethyl-1H-pyrazol-1-yl)-2-((R)-1-((S)-1-methylpyrrolidin-2-yl)ethoxy)-N-(1-(pyrimidin-5-yl)ethyl)pyrimidine-4-carboxamide;
6-((Sₐ)-4-(2-amino-3-cyano-7-fluorobenzo[b]thiophene-4-yl)-3,5-dimethyl-1H-pyrazol-1-yl)-2-((S)-1-((R)-1-methylpyrrolidin-2-yl)ethoxy)-N-(1-(pyrimidin-5-yl)ethyl)pyrimidine-4-carboxamide;
2-amino-4-(5-ethyl-3-methyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((3aS,6aS)-1-methylhexahydropyrrolo[3,4-b]pyrrol-5(1H)-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((3aR,6aR)-1-methylhexahydropyrrolo[3,4-b]pyrrol-5(H)-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((3aS,6aS)-1-methylhexahydropyrrolo[3,4-b]pyrrol-5(H)-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((3aR,6aR)-1-methylhexahydropyrrolo[3,4-b]pyrrol-5(H)-yl)pyrimidin-4-yl)-1H-pyrazol-4-yl)-7-(Rₐ)-fluorothieno[3,2-c]pyridine-3-carbonitrile;
2-amino-4-(5-ethyl-3-methyl-1-(6-(2-methyl-1-oxoisoindolin-5-yl)-2-((3aS,6aS)-1-methylhexahydropyrrolo[3,4-b]pyrrol-5(1H)-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((3aR,6aR)-1-methylhexahydropyrrolo[3,4-b]pyrrol-5(1H)-yl)pyrimidin-4-yl)-1H-pyrazol-4-yl)-7-(Sₐ)-fluorothieno[3,2-c]pyridine-3-carbonitrile;
5-(6-(4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-methylpicolinamide;
5-(6-(4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-N-methylpicolinamide;
5-(6-(4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-N-methylpicolinamide;
5-(6-(4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-N-methylpicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((S)-1-((S)-1-methlpyrrolidin-2-yl)ethoxy)pyrimidin-4-yl)-N-methylpicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-N-methylpicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-N-methylpicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-N-methylpicolinamide;
5-(6-((Sₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-methylpicolinamide;
5-(6-((Sₐ)-4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-N-methylpicolinamide;
5-(6-((Sₐ)-4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-N-methylpicolinamide;
5-(6-((Sₐ)-4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-N-methylpicolinamide;
4-(6-(4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-2-fluoro-N-methylbenzamide;
4-(6-(4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-2-fluoro-N-methylbenzamide;
4-(6-(4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-2-fluoro-N-methylbenzamide;
4-(6-(4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-2-fluoro-N-methylbenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-2-fluoro-N-methylbenzamide;
4-(6-((R)-4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-2-fluoro-N-methylbenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-2-fluoro-N-methylbenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-2-fluoro-N-methylbenzamide;
4-(6-((Sₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-2-fluoro-N-methylbenzamide;
4-(6-((Sₐ)-4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-2-fluoro-N-methylbenzamide;
4-(6-((Sₐ)-4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-2-fluoro-N-methylbenzamide;
4-(6-((S$)-4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-2-fluoro-N-methylbenzamide;
4-(6-(4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-cyclopropyl-2-fluorobenzamide;
4-(6-(4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-N-cyclopropyl-2-fluorobenzamide;
4-(6-(4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-N-cyclopropyl-2-fluorobenzamide;
4-(6-(4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-N-cyclopropyl-2-fluorobenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-cyclopropyl-2-fluorobenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-N-cyclopropyl-2-fluorobenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-N-cyclopropyl-2-fluorobenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-N-cyclopropyl-2-fluorobenzamide;
4-(6-((Sₐ)-4-(2-amino-3-cyano-7-fluorothieno[3,2-c]pyridin-4-yl)-5-ethyl-3-methyl-1H-pyrazol-1-yl)-2-((S)-1-((S)-1-methylpyrrolidin-2-yl)ethoxy)pyrimidin-4-yl)-N-cyclopropyl-2-fluorobenzamide;
4-(6-((S)-4-(2-amino-3-cyano-7-fluorothieno[3,2-c]pyridin-4-yl)-5-ethyl-3-methyl-1H-pyrazol-1-yl)-2-((R)-1-((R)-1-methylpyrrolidin-2-yl)ethoxy)pyrimidin-4-yl)-N-cyclopropyl-2-fluorobenzamide;
4-(6-((S)-4-(2-amino-3-cyano-7-fluorothieno[3,2-c]pyridin-4-yl)-5-ethyl-3-methyl-1H-pyrazol-1-yl)-2-((S)-1-((R)-1-methylpyrrolidin-2-yl)ethoxy)pyrimidin-4-yl)-N-cyclopropyl-2-fluorobenzamide;
4-(6-((Sₐ)-4-(2-amino-3-cyano-7-fluorothieno[3,2-c]pyridin-4-yl)-5-ethyl-3-methyl-1H-pyrazol-1-yl)-2-((R)-1-((S)-1-methylpyrrolidin-2-yl)ethoxy)pyrimidin-4-yl)-N-cyclopropyl-2-fluorobenzamide;
5-(6-(4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-3-fluoro-N-methylpicolinamide;
5-(6-(4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-3-fluoro-N-methylpicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-3-fluoro-N-methylpicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-3-fluoro-N-methylpicolinamide;
5-(6-((Sₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-3-fluoro-N-methylpicolinamide;
5-(6-((Sₐ)-4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-3-fluoro-N-methylpicolinamide;
4-(6-(4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-2-fluorobenzamide;
4-(6-(4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-N-cyclopropyl-2-fluorobenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-2-fluorobenzamide;
4-(6-((Rₐ)-4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-N-cyclopropyl-2-fluorobenzamide;
4-(6-((Sₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-2-fluorobenzamide;
4-(6-((S$)-4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-N-cyclopropyl-2-fluorobenzamide;
5-(6-(4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-3-fluoropicolinamide;
5-(6-(4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-N-cyclopropyl-3-fluoropicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-3-fluoropicolinamide;
5-(6-((Rₐ)-4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-N-cyclopropyl-3-fluoropicolinamide;
5-(6-((Sₐ)-4-(2-amino-3-cyano-7-fluorothieno[3,2-c]pyridin-4-yl)-5-ethyl-3-methyl-1H-pyrazol-1-yl)-2-((3aS,6aS)-1-methylhexahydropyrrolo[3,4-b]pyrrol-5(1H)-yl)pyrimidin-4-yl)-N-cyclopropyl-3-fluoropicolinamide;
5-(6-((S$)-4-(2-amino-3-cyano-7-fluorothieno[3,2-c]pyridin-4-yl)-5-ethyl-3-methyl-1H-pyrazol-1-yl)-2-((3aR,6aR)-1-methylhexahydropyrrolo[3,4-b]pyrrol-5(1H)-yl)pyrimidin-4-yl)-N-cyclopropyl-3-fluoropicolinamide;
2-amino-4-(5-ethyl-1-(6-(2-methyl-1-oxoisoindolin-5-yl)-2-((S)-1-((S)-1-methylpyrrolidin-2-yl)ethoxy)pyrimidin-4-yl)-3-(trifluoromethyl)-1H-pyrazol-4-yl)-7-fluorothieno[3,2-c]pyridine-3-carbonitrile;
2-amino-4-(5-ethyl-1-(6-(2-methyl-1-oxoisoindolin-5-yl)-2-((S)-1-((R)-1-methylpyrrolidin-2-yl)ethoxy)pyrimidin-4-yl)-3-(trifluoromethyl)-1H-pyrazol-4-yl)-7-fluorothieno[3,2-c]pyridine-3-carbonitrile;
2-amino-4-(5-ethyl-1-(6-(2-methyl-1-oxoisoindolin-5-yl)-2-((R)-1-((S)-1-methylpyrrolidin-2-yl)ethoxy)pyrimidin-4-yl)-3-(trifluoromethyl)-1H-pyrazol-4-yl)-7-fluorothieno[3,2-c]pyridine-3-carbonitrile;
2-amino-4-(5-ethyl-1-(6-(2-methyl-1-oxoisoindolin-5-yl)-2-((R)-1-((R)-1-methylpyrrolidin-2-yl)ethoxy)pyrimidin-4-yl)-3-(trifluoromethyl)-1H-pyrazol-4-yl)-7-fluorothieno[3,2-c]pyridine-3-carbonitrile;
2-amino-4-(5-ethyl-1-(6-(2-methyl-1-oxoisoindolin-5-yl)-2-((S)-1-((S)-1-methylpyrrolidin-2-yl)ethoxy)pyrimidin-4-yl)-3-(trifluoromethyl)-1H-pyrazol-4-yl)-(Rₐ)-7-fluorothieno[3,2-c]pyridine-3-carbonitrile;
2-amino-4-(5-ethyl-1-(6-(2-methyl-1-oxoisoindolin-5-yl)-2-((S)-1-((S)-1-methylpyrrolidin-2-yl)ethoxy)pyrimidin-4-yl)-3-(trifluoromethyl)-1H-pyrazol-4-yl)-(Sₐ)-7-fluorothieno[3,2-c]pyridine-3-carbonitrile;
2-amino-4-(5-ethyl-1-(6-(2-methyl-1-oxoisoindolin-5-yl)-2-((S)-1-((R)-1-methylpyrrolidin-2-yl)ethoxy)pyrimidin-4-yl)-3-(trifluoromethyl)-1H-pyrazol-4-yl)-(Rₐ)-7-fluorothieno[3,2-c]pyridine-3-carbonitrile;
2-amino-4-(5-ethyl-1-(6-(2-methyl-1-oxoisoindolin-5-yl)-2-((S)-1-((R)-1-methylpyrrolidin-2-yl)ethoxy)pyrimidin-4-yl)-3-(trifluoromethyl)-1H-pyrazol-4-yl)-(Sₐ)-7-fluorothieno[3,2-c]pyridine-3-carbonitrile;
2-amino-4-(5-ethyl-1-(6-(2-methyl-1-oxoisoindolin-5-yl)-2-((R)-1-((S)-1-methylpyrrolidin-2-yl)ethoxy)pyrimidin-4-yl)-3-(trifluoromethyl)-1H-pyrazol-4-yl)-(Rₐ)-7-fluorothieno[3,2-c]pyridine-3-carbonitrile;
2-amino-4-(5-ethyl-1-(6-(2-methyl-1-oxoisoindolin-5-yl)-2-((R)-1-((S)-1-methylpyrrolidin-2-yl)ethoxy)pyrimidin-4-yl)-3-(trifluoromethyl)-1H-pyrazol-4-yl)-(Sₐ)-7-fluorothieno[3,2-c]pyridine-3-carbonitrile;
2-amino-4-(5-ethyl-1-(6-(2-methyl-1-oxoisoindolin-5-yl)-2-((R)-1-((R)-1-methylpyrrolidin-2-yl)ethoxy)pyrimidin-4-yl)-3-(trifluoromethyl)-1H-pyrazol-4-yl)-(Rₐ)-7-fluorothieno[3,2-c]pyridine-3-carbonitrile;
2-amino-4-(5-ethyl-1-(6-(2-methyl-1-oxoisoindolin-5-yl)-2-((R)-1-((R)-1-methylpyrrolidin-2-yl)ethoxy)pyrimidin-4-yl)-3-(trifluoromethyl)-1H-pyrazol-4-yl)-(Sₐ)-7-fluorothieno[3,2-c]pyridine-3-carbonitrile;
2-amino-4-(3,5-diethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3,5-diethyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3,5-diethyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3,5-diethyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-7-fluorothieno[3,2-c]pyridine-3-carbonitrile;
2-amino-4-(3,5-diethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(3,5-diethyl-1-(6-(2-methyl-1-oxoisoindolin-5-yl)-2-((S)-1-((S)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
2-amino-4-(3,5-diethyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(3,5-diethyl-1-(6-(2-methyl-1-oxoisoindolin-5-yl)-2-((S)-1-((R)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
2-amino-4-(3,5-diethyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(3,5-diethyl-1-(6-(2-methyl-1-oxoisoindolin-5-yl)-2-((R)-1-((S)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
2-amino-4-(3,5-diethyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-(Rₐ)-7-fluorothieno[3,2-c]pyridine-3-carbonitrile;
2-amino-4-(3,5-diethyl-1-(6-(2-methyl-1-oxoisoindolin-5-yl)-2-((R)-1-((R)-1-methylpyrrolidin-2-yl)ethoxy)pyrimidin-4-yl)-1H-pyrazol-4-yl)-(Sₐ)-7-fluorothieno[3,2-c]pyridine-3-carbonitrile;
(S)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-7-fluorothieno[3,2-c]pyridine-3-carbonitrile;
(R)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-7-fluorothieno[3,2-c]pyridine-3-carbonitrile;
(S)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-(Rₐ)-7-fluorothieno[3,2-c]pyridine-3-carbonitrile;
(S)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-(Sₐ)-7-fluorothieno[3,2-c]pyridine-3-carbonitrile;
(R)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-(Rₐ)-7-fluorothieno[3,2-c]pyridine-3-carbonitrile;
(R)-2-amino-4-(5-ethyl-3-methyl-1-(6-(2-methyl-1-oxoisoindolin-5-yl)-2-((1-methylpyrrolidin-2-yl)methoxy)pyrimidin-4-yl)-1H-pyrazol-4-yl)-(Sₐ)-7-fluorothieno[3,2-c]pyridine-3-carbonitrile;
(S)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
(R)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
(S)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile;
(S)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile;
(R)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile;
(R)-2-amino-4-(1-(2-(3-(dimethylamino)pyrrolidin-1-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((1 S,5S)-6-methyl-3,6-diazabicyclo[3.2.0]heptan-3-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((1 S,5R)-6-methyl-3,6-diazabicyclo[3.2.0]heptan-3-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((1R,5S)-6-methyl-3,6-diazabicyclo[3.2.0]heptan-3-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl-2-((1R,5R)-6-methyl-3,6-diazabicyclo[3.2.0]heptan-3-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl-2-((1 S,5S)-6-methyl-3,6-diazabicyclo[3.2.0]heptan-3-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((1 S,5R)-6-methyl-3,6-diazabicyclo[3.2.0]heptan-3-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((1R,5S)-6-methyl-3,6-diazabicyclo[3.2.0]heptan-3-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((1R,5R)-6-methyl-3,6-diazabicyclo[3.2.0]heptan-3-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl-2-((1 S,5S)-6-methyl-3,6-diazabicyclo[3.2.0]heptan-3-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((1 S,5R)-6-methyl-3,6-diazabicyclo[3.2.0]heptan-3-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((1R,5S)-6-methyl-3,6-diazabicyclo[3.2.0]heptan-3-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((1R,5R)-6-methyl-3,6-diazabicyclo[3.2.0]heptan-3-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aS,7aS)-1-methyloctahydro-6H-pyrrolo[3,4-b]pyridin-6-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((4aS,7aR)-1-methyloctahydro-6H-pyrrolo[3,4-b]pyridin-6-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((4aR,7aS)-1-methyloctahydro-6H-pyrrolo[3,4-b]pyridin-6-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((4aR,7aR)-1-methyloctahydro-6H-pyrrolo[3,4-b]pyridin-6-yl)pyrimidin-4-yl)-1H-pyrazol-4-yl)-7-fluorothieno[3,2-c]pyridine-3-carbonitrile;
2-amino-4-(5-ethyl-3-methyl-1-(6-(2-methyl-1-oxoisoindolin-5-yl)-2-((4aS,7aS)-1-methyloctahydro-6H-pyrrolo[3,4-b]pyridin-6-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aS,7aS)-1-methyloctahydro-6H-pyrrolo[3,4-b]pyridin-6-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aS,7aR)-1-methyloctahydro-6H-pyrrolo[3,4-b]pyridin-6-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aS,7aR)-1-methyloctahydro-6H-pyrrolo[3,4-b]pyridin-6-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aR,7aS)-1-methyloctahydro-6H-pyrrolo[3,4-b]pyridin-6-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aR,7aS)-1-methyloctahydro-6H-pyrrolo[3,4-b]pyridin-6-yl)pyrimidin-4-yl)-1H-pyrazol-4-yl)-(Sₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aR,7aR)-1-methyloctahydro-6H-pyrrolo[3,4-b]pyridin-6-yl)pyrimidin-4-yl)-1H-pyrazol-4-yl)-(Rₐ)-7-fluorothieno[3,2-c]pyridine-3-carbonitrile;
2-amino-4-(5-ethyl-3-methyl-1-(6-(2-methyl-1-oxoisoindolin-5-yl)-2-((4aR,7aR)-1-methyloctahydro-6H-pyrrolo[3,4-b]pyridin-6-yl)pyrimidin-4-yl)-1H-pyrazol-4-yl)-(Sₐ)-7-fluorothieno[3,2-c]pyridine-3-carbonitrile;
2-amino-4-(1-(2-((3aS,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
2-amino-4-(1-(2-((3aS,6aR)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
2-amino-4-(1-(2-((3aR,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
2-amino-4-(1-(2-((3aR,6aR)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-7-fluorothieno[3,2-c]pyridine-3-carbonitrile;
2-amino-4-(1-(2-((3aS,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile;
2-amino-4-(1-(2-((3aS,6aR)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile;
2-amino-4-(1-(2-((3aR,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile;
2-amino-4-(1-(2-((3aR,6aR)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Rₐ)-7-fluorothieno[3,2-c]pyridine-3-carbonitrile;
2-amino-4-(1-(2-((3aS,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile;
2-amino-4-(1-(2-((3aS,6aR)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile;
2-amino-4-(1-(2-((3aR,6aS)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile; and
2-amino-4-(1-(2-((3aR,6aR)-1-cyclopropylhexahydropyrrolo[3,4-b]pyrrol-5(1H)-yl)-6-(2-methyl-1-oxoisoindolin-5-yl)pyrimidin-4-yl)-5-ethyl-3-methyl-1H-pyrazol-4-yl)-(Sₐ)-7-fluorothieno[3,2-c]pyridine-3-carbonitrile;
and pharmaceutically acceptable salts thereof.

25. A pharmaceutical composition comprising the compound or pharmaceutically acceptable salt thereof according to claim 1, and at least one pharmaceutically acceptable carrier or excipient.

26. A method of inhibiting KRAS activity, the method comprising contacting the compound or pharmaceutically acceptable salt thereof according to claim 1, with KRAS.

27-30. (canceled)

31. A method of treating a disease or disorder associated with activity of KRAS, the method comprising administering to a patient in need thereof a therapeutically effective amount of the compound or pharmaceutically acceptable salt thereof according to claim 1.

32. A method of treating a disease or disorder associated with activity of a KRAS protein harboring a G12C, G12D, or G12V mutation, the method comprising administering to a patient in need thereof a therapeutically effective amount of the compound or pharmaceutically acceptable salt thereof according to claim 1.

33-34. (canceled)

35. A method for treating a cancer in a patient, the method comprising administering to the patient a therapeutically effective amount of the compound or pharmaceutically acceptable salt thereof according to claim 1.

36. The method of claim 35, wherein the cancer is selected from a carcinoma, a hematological cancer, a sarcoma, and glioblastoma.

37. The method of claim 36, wherein the cancer is a hematological cancer selected from myeloproliferative neoplasms, myelodysplastic syndrome, chronic and juvenile myelomonocytic leukemia, acute myeloid leukemia, acute lymphocytic leukemia, and multiple myeloma.

38. The method of claim 36, wherein the cancer is a carcinoma selected from pancreatic, colorectal, lung, bladder, gastric, esophageal, breast, head and neck, cervical, skin, and thyroid cancers.

39. The method of claim 35, wherein abnormally proliferating cells of the cancer comprise KRAS having a G12C, G12D, or G12V mutation.

40-41. (canceled)

42. A method of treating an immunological or inflammatory disorder comprising administering to a patient in need thereof a therapeutically effective amount of the compound or pharmaceutically acceptable salt thereof according to claim 1.

43. The method of claim 42, wherein the immunological or inflammatory disorder is associated with activity of KRAS.

44. The method of claim 42, wherein the immunological or inflammatory disorder is associated with activity of KRAS having a G12C, G12D, or G12V mutation.

45-46. (canceled)

47. The method of claim 42, wherein the immunological or inflammatory disorder is Ras-associated lymphoproliferative disorder or juvenile myelomonocytic leukemia caused by a somatic mutation of KRAS.

48-53. (canceled)
